(19) United States
(12) Patent Application Publication
(10) Pub. No.: US 2003/0171311 A1
(43) Pub. Date: Sep. 11, 2003

(54) Enzymatic nucleic acid treatment of diseases or conditions related to hepatitis C virus infection

(76) Inventors: Lawrence Blatt, Boulder, CO (US); James McSwiggen, Boulder, CO (US); Elisabeth Roberts, Federal Heights, CO (US); Pamela A. Pavco, Lafayette, CO (US); Dennis MacJack, Arvada, CO (US)

Correspondence Address: MCDONNELL BOEHNEN HULBERT & BERGHOFF, 300 SOUTH WACKER DRIVE, SUITE 3200, CHICAGO, IL, 60606, US

(21) Appl. No.: 09/817,879

(22) Filed: Mar. 26, 2001

Related U.S. Application Data
(63) Continuation-in-part of application No. 09/740,332, filed on Dec. 18, 2000.
Application No. 09/740,332 is a continuation-in-part of application No. 09/611,931, filed on Jul. 7, 2000.
Application No. 09/611,931 is a continuation-in-part of application No. 09/504,231, filed on Feb. 15, 2000.
Application No. 09/504,231 is a continuation-in-part of application No. 09/274,553, filed on Mar. 23, 1999.
Application No. 09/274,553 is a continuation-in-part of application No. 09/257,608, filed on Feb. 25, 1999, now abandoned.
(60) Provisional application No. 60/100,842, filed on Sep. 18, 1998.
(60) Provisional application No. 60/083,217, filed on Apr. 27, 1998.

(51) Int. Cl. A61K 48/00; C07H 21/04

(52) U.S. Cl. 514/44; 536/23.1

(57) ABSTRACT

The present invention relates to compounds, including enzymatic nucleic acid molecules, ribozymes, DNAzymes, nuclease activating compounds and chimeras such as 2′,5′-adenylates, that modulate the expression and/or replication of hepatitis C virus (HCV).

This patent application is a continuation-in-part of Blatt et al., U.S. Ser. No. (09/740,332), filed Dec. 18, 2000, which is a continuation-in-part of Blatt et al., U.S. Ser. No. (09/611,931), filed Jul. 7, 2000, which is a continuation-in-part of Blatt et al., Ser. No. 09/504,321, filed Feb. 15, 2000, which is a continuation-in-part of Blatt et al., U.S. Ser. No. 09/274,553, filed Mar. 23, 1999, which is a continuation-in-part of Blatt et al., U.S. Ser. No. 09/257,608, filed Feb. 24, 1999 (abandoned), which claims priority from Blatt et al., U.S. Ser. No. 60/100,842, filed Sep. 18, 1998, and McSwiggen et al., U.S. Ser. No. 60/083,217 filed Apr. 27, 1998, all of these earlier applications are entitled “ENZYMATIC NUCLEIC ACID TREATMENT OF DISEASES OR CONDITIONS RELATED TO HEPATITIS C VIRUS INFECTION”. Each of these applications are hereby incorporated by reference herein in their entirety including the drawings.

The Sequence Listing file named “MBHBOO-80sequenceListing.ST25” submitted on Compact Disc-Recordable (CD-R) medium (“010323_{—}1557”) in compliance with 37 C.F.R. §1.52(e) is incorporated herein by reference.

### TECHNICAL FIELD OF THE INVENTION

This invention relates to methods and reagents for the treatment of diseases or conditions relating to the hepatitis C virus infection.

### BACKGROUND OF THE INVENTION

The following is a discussion of relevant art, none of which is admitted to be prior art to the present invention.

In 1989, the Hepatitis C Virus (HCV) was determined to be an RNA virus and was identified as the causative agent of most non-A non-B viral Hepatitis (Choo et al., Science. 1989; 244:359-362). Unlike retroviruses such as HIV, HCV does not have a DNA replication phase and no integrated forms of the viral genome into the host chromosome have been detected (Houghton et al., Hepatology 1991;14:381-388). Rather, replication of the coding (plus) strand is mediated by the production of a replicative (minus) strand leading to the generation of several copies of plus strand HCV RNA. The genome consists of a single, large, open-reading frame that is translated into a polyprotein (Kato et al., FEBS Letters. 1991; 280: 325-328). This polyprotein subsequently undergoes post-translational cleavage, producing several viral proteins (Leinbach et al., Virology. 1994: 204:163-169).

Examination of the 9.5-kilobase genome of HCV has demonstrated that the viral nucleic acid can mutate at a high rate (Smith et al., Mol. Evol 1997 45:238-246). This rate of mutation has led to the evolution of several distinct genotypes of HCV that share approximately 70% sequence identity (Simmonds et al., J. Gen. Virol. 1994; 75 :1053-1061). It is important to note that these sequences are evolutionarily quite distant. For example, the genetic identity between humans and primates such as the chimpanzee is approximately 98%. In addition, it has been demonstrated that an HCV infection in an individual patient is composed of several distinct and evolving quasispecies that have 98% identity at the RNA level. Thus, the HCV genome is hypervariable and continuously changing. Although the HCV genome is hypervariable, there are 3 regions of the genome that are highly conserved. These conserved sequences occur in the 5′ and 3′ non-coding regions as well as the 5′-end of the core protein coding region and are thought to be vital for HCV RNA replication as well as translation of the HCV polyprotein. Thus, therapeutic agents that target these conserved HCV genomic regions can have a significant impact over a wide range of HCV genotypes. Moreover, it is unlikely that drug resistance will occur with enzymatic nucleic acids specific to conserved regions of the HCV genome. In contrast, therapeutic modalities that target inhibition of enzymes such as the viral proteases or helicase are likely to result in the selection for drug resistant strains since the RNA for these viral encoded enzymes is located in the hypervariable portion of the HCV genome.

After initial exposure to HCV, the patient will experience a transient rise in liver enzymes, which indicates that inflammatory processes are occurring (Alter et al., IN: Seeff LB, Lewis JH, eds. Current Perspectives in Hepatology. New York: Plenum Medical Book Co; 1989:83-89). This elevation in liver enzymes will occur at least 4 weeks after the initial exposure and can last for up to two months (Farci et al., New England Journal of Medicine. 1991:325:98-104). Prior to the rise in liver enzymes, it is possible to detect HCV RNA in the patient's serum using RT-PCR analysis (Takahashi et al., American Journal of Gastroenterology. 1993:88:2:240-243). This stage of the disease is called the acute stage and usually goes undetected since 75% of patients with acute viral hepatitis from HCV infection are asymptomatic. The remaining 25% of these patients develop jaundice or other symptoms of hepatitis.

Acute HCV infection is a benign disease, however, and as many as 80% of acute HCV patients progress to chronic liver disease as evidenced by persistent elevation of serum alanine aminotransferase (ALT) levels and by continual presence of circulating HCV RNA (Sherlock, Lancet 1992; 339:802). The natural progression of chronic HCV infection over a 10 to 20 year period leads to cirrhosis in 20 to 50% of patients (Davis et al., Infectious Agents and Disease 1993;2:150:154) and progression of HCV infection to hepatocellular carcinoma has been well documented (Liang et al., Hepatology. 1993; 18:1326-1333; Tong et al., Western Journal of Medicine, 1994; Vol. 160, No. 2: 133-138). There have been no studies that have determined sub-populations that are most likely to progress to cirrhosis and/or hepatocellular carcinoma, thus all patients have equal risk of progression.

It is important to note that the survival for patients diagnosed with hepatocellular carcinoma is only 0.9 to 12.8 months from initial diagnosis (Takahashi et al., American Journal of Gastroenterology. 1993:88:2:240-243). Treatment of hepatocellular carcinoma with chemotherapeutic agents has not proven effective and only 10% of patients will benefit from surgery due to extensive tumor invasion of the liver (Trinchet et al., Presse Medicine. 1994:23:831-833). Given the aggressive nature of primary hepatocellular carcinoma, the only viable treatment alternative to surgery is liver transplantation (Pichlmayr et al., Hepatology. 1994:20:33S-40S).

Upon progression to cirrhosis, patients with chronic HCV infection present with clinical features, which are common to clinical cirrhosis regardless of the initial cause (D'Amico et al., Digestive Diseases and Sciences. 1986;31:5: 468-475). These clinical features can include: bleeding esophageal varices, ascites, jaundice, and encephalopathy (Zakim D, Boyer TD. Hepatology a textbook of liver disease. Second Edition Volume 1. 1990 W.B. Saunders Company. Philadelphia). In the early stages of cirrhosis, patients are classified as compensated meaning that although liver tissue damage has occurred, the patient's liver is still able to detoxify metabolites in the blood-stream. In addition, most patients with compensated liver disease are asymptomatic and the minority with symptoms report only minor symptoms such as dyspepsia and weakness. In the later stages of cirrhosis, patients are classified as decompensated meaning that their ability to detoxify metabolites in the bloodstream is diminished and it is at this stage that the clinical features described above will present.

In 1986, D'Amico et al. described the clinical manifestations and survival rates in 1155 patients with both alcoholic and viral associated cirrhosis (D'Amico supra). Of the 1155 patients, 435 (37%) had compensated disease although 70% were asymptomatic at the beginning of the study. The remaining 720 patients (63%) had decompensated liver disease with 78% presenting with a history of ascites, 31% with jaundice, 17% had bleeding and 16% had encephalopathy. Hepatocellular carcinoma was observed in six (0.5%) patients with compensated disease and in 30 (2.6%) patients with decompensated disease.

Over the course of six years, the patients with compensated cirrhosis developed clinical features of decompensated disease at a rate of 10% per year. In most cases, ascites was the first presentation of decompensation. In addition, hepatocellular carcinoma developed in 59 patients who initially presented with compensated disease by the end of the six-year study.

With respect to survival, the D'Amico study indicated that the five-year survival rate for all patients on the study was only 40%. The six-year survival rate for the patients who initially had compensated cirrhosis was 54% while the six-year survival rate for patients who initially presented with decompensated disease was only 21%. There were no significant differences in the survival rates between the patients who had alcoholic cirrhosis and the patients with viral related cirrhosis. The major causes of death for the patients in the D'Amico study were liver failure in 49%; hepatocellular carcinoma in 22%; and, bleeding in 13% (D'Amico supra).

Chronic Hepatitis C is a slowly progressing inflammatory disease of the liver, mediated by a virus (HCV) that can lead to cirrhosis, liver failure and/or hepatocellular carcinoma over a period of 10 to 20 years. In the US, it is estimated that infection with HCV accounts for 50,000 new cases of acute hepatitis in the United States each year (NIH Consensus Development Conference Statement on Management of Hepatitis C March 1997). The prevalence of HCV in the United States is estimated at 1.8% and the CDC places the number of chronically infected Americans at approximately 4.5 million people. The CDC also estimates that up to 10,000 deaths per year are caused by chronic HCV infection. The prevalence of HCV in the United States is estimated at 1.8% and the CDC places the number of chronically infected Americans at approximately 4.5 million people. The CDC also estimates that up to 10,000 deaths per year are caused by chronic HCV infection.

Numerous well controlled clinical trials using interferon (IFN-alpha) in the treatment of chronic HCV infection have demonstrated that treatment three times a week results in lowering of serum ALT values in approximately 50% (range 40% to 70%) of patients by the end of 6 months of therapy (Davis et al., New England Journal of Medicine 1989; 321:1501-1506; Marcellin et al., Hepatology. 1991; 13:393-397; Tong et al., Hepatology 1997:26:747-754; Tong et al., Hepatology 1997 26(6): 1640-1645). However, following cessation of interferon treatment, approximately 50% of the responding patients relapsed, resulting in a “durable” response rate as assessed by normalization of serum ALT concentrations of approximately 20 to 25%.

In recent years, direct measurement of the HCV RNA has become possible through use of either the branched-DNA or Reverse Transcriptase Polymerase Chain Reaction (RT-PCR) analysis. In general, the RT-PCR methodology is more sensitive and leads to more accurate assessment of the clinical course (Tong et al., supra). Studies that have examined six months of type 1 interferon therapy using changes in HCV RNA values as a clinical endpoint have demonstrated that up to 35% of patients will have a loss of HCV RNA by the end of therapy (Marcellin et al., supra). However, as with the ALT endpoint, about 50% of the patients relapse six months following cessation of therapy resulting in a durable virologic response of only 12% (Marcellin et al., supra). Studies that have examined 48 weeks of therapy have demonstrated that the sustained virological response is up to 25% (NIH consensus statement: 1997). Thus, standard of care for treatment of chronic HCV infection with type 1 interferon is now 48 weeks of therapy using changes in HCV RNA concentrations as the primary assessment of efficacy (Hoofnagle et al., New England Journal of Medicine 1997; 336(5) 347-356).

Side effects resulting from treatment with type 1 interferons can be divided into four general categories, which include 1. Influenza-like symptoms; 2. Neuropsychiatric; 3. Laboratory abnormalities; and, 4. Miscellaneous (Dusheiko et al., Journal of Viral Hepatitis. 1994:1:3-5). Examples of influenza-like symptoms include; fatigue, fever; myalgia; malaise; appetite loss; tachycardia; rigors; headache and arthralgias. The influenza-like symptoms are usually short-lived and tend to abate after the first four weeks of dosing (Dushieko et al., supra). Neuropsychiatric side effects include: irritability, apathy; mood changes; insomnia; cognitive changes and depression. The most important of these neuropsychiatric side effects is depression and patients who have a history of depression should not be given type 1 interferon. Laboratory abnormalities include; reduction in myeloid cells including granulocytes, platelets and to a lesser extent red blood cells. These changes in blood cell counts rarely lead to any significant clinical sequellae (Dushieko et al., supra). In addition, increases in triglyceride concentrations and elevations in serum alanine and aspartate aminotransferase concentration have been observed. Finally, thyroid abnormalities have been reported. These thyroid abnormalities are usually reversible after cessation of interferon therapy and can be controlled with appropriate medication while on therapy. Miscellaneous side effects include nausea; diarrhea; abdominal and back pain; pruritus; alopecia; and rhinorrhea. In general, most side effects will abate after 4 to 8 weeks of therapy (Dushieko et al, supra).

Type 1 Interferon is a key constituent of many treatment programs for chronic HCV infection. Treatment with type 1 interferon induces a number of genes and results in an antiviral state within the cell. One of the genes induced is 2′, 5′ oligoadenylate synthetase, an enzyme that synthesizes short 2′, 5′ oligoadenylate (2-5A) molecules. Nascent 2-5A subsequently activates a latent RNase, RNase L, which in turn nonspecifically degrades viral RNA.

Welch et al., Gene Therapy 1996 3(11): 994-1001 describe in vitro an in vivo studies with two vector expressed hairpin ribozymes targeted against hepatitis C virus.

Sakamoto et al., J. Clinical Investigation 1996 98(12): 2720-2728 describe intracellular cleavage of hepatitis C virus RNA and inhibition of viral protein translation by certain vector expressed hammerhead ribozymes.

Lieber et al., J. Virology 1996 70(12): 8782-8791 describe elimination of hepatitis C virus RNA in infected human hepatocytes by adenovirus-mediated expression of certain hammerhead ribozymes.

Ohkawa et al., 1997, J. Hepatology, 27; 78-84, describe in vitro cleavage of HCV RNA and inhibition of viral protein translation using certain in vitro transcribed hammerhead ribozymes.

Barber et al., International PCT Publication No. WO 97/32018, describe the use of an adenovirus vector to express certain anti-hepatitis C virus hairpin ribozymes.

Kay et al., International PCT Publication No. WO 96/18419, describe certain recombinant adenovirus vectors to express anti-HCV hammerhead ribozymes.

Yamada et al., Japanese Patent Application No. JP 07231784 describe a specific poly-(L)-lysine conjugated hammerhead ribozyme targeted against HCV.

Draper, U.S. Pat. Nos. 5,610,054 and 5,869,253, describes enzymatic nucleic acid molecules capable of inhibiting replication of HCV.

Macejak et al., 2000, Hepatology, 31, 769-776, describe enzymatic nucleic acid molecules capable of inhibiting replication of HCV.

Weifeng and Torrence, 1997, Nucleosides and Nucleotides, 16, 7-9, describe the synthesis of 2-5A antisense chimeras with various non-nucleoside components.

Torrence et al, U.S. Pat. No. 5,583,032 describe targeted cleavage of RNA using an antisense oligonucleotide linked to a 2′,5′-oligoadenylate activator of RNase L.

Suhadolnik and Pfleiderer, U.S. Pat. Nos. 5,863,905; 5,700,785; 5,643,889; 5,556,840; 5,550,111; 5,405,939; 5,188,897; 4,924,624; and 4,859,768 describe specific internucleotide phosphorothioate 2′,5′-oligoadenlyates and 2′,5′-oligoadenlyate conjugates.

Budowsky et al., U.S. Pat. No. 5,962,431 describe a method of treating papillomavirus using specific 2′,5′-oligoadenylates.

Torrence et al., International PCT publication No. WO 00/14219, describe specific peptide nucleic acid 2′,5′-oligoadenylate chimeric molecules.

Stinchcomb et al., U.S. Pat. No. 5,817,796, describe C-myb ribozymes having 2′-5′-Linked Adenylate Residues.

### SUMMARY OF THE INVENTION

This invention relates to enzymatic nucleic acid molecules directed to cleave RNA species of hepatitis C virus (HCV) and/or encoded by the HCV. In particular, applicant describes the selection and function of enzymatic nucleic acid molecules capable of specifically cleaving HCV RNA. The invention further relates to compounds and chimeric molecules comprising nuclease activating activity. The invention also relates to compositions and methods for the cleavage of RNA using these nuclease activating compounds and chimeras. Nucleic acid molecules, nuclease activating compounds and chimeras, and compositions and methods of the invention can be used to treat diseases associated with HCV infection.

Due to the high sequence variability of the HCV genome, selection of nucleic acid molecules and nuclease activating compounds and chimeras for broad therapeutic applications would likely involve the conserved regions of the HCV genome. Specifically, the present invention describes nucleic acid molecules that cleave the conserved regions of the HCV genome. The invention further describes compounds and chimeric molecules that activate cellular nucleases that cleave HCV RNA, including conserved regions of the HCV genome. Examples of conserved regions of the HCV genome include but are not limited to the 5′-Non Coding Region (NCR), the 5′-end of the core protein coding region, and the 3′-NCR. HCV genomic RNA contains an internal ribosome entry site (IRES) in the 5′-NCR which mediates translation independently of a 5′-cap structure (Wang et al., 1993, J. Virol., 67, 3338-44). The full-length sequence of the HCV RNA genome is heterologous among clinically isolated subtypes, of which there are at least 15 (Simmonds, 1995, Hepatology, 21, 570-583), however, the 5′-NCR sequence of HCV is highly conserved across all known subtypes, most likely to preserve the shared IRES mechanism (Okamoto et al., 1991, J. General Virol., 72, 2697-2704) In general, enzymatic nucleic acid molecules and nuclease activating compounds, and chimeras that cleave sites located in the 5′ end of the HCV genome are expected to block translation while nucleic acid molecules and nuclease activating compounds, and chimeras that cleave sites located in the 3′ end of the genome would be expected to block RNA replication. Therefore, one nucleic acid molecule, compound, or chimera can be designed to cleave all the different isolates of HCV. Enzymatic nucleic acid molecules and nuclease activating compounds, and chimeras designed against conserved regions of various HCV isolates can enable efficient inhibition of HCV replication in diverse patient populations and can ensure the effectiveness of the nucleic acid molecules and nuclease activating compounds, and chimeras against HCV quasi species which evolve due to mutations in the non-conserved regions of the HCV genome.

In one embodiment, the invention features the use of an enzymatic nucleic acid molecule, preferably in the hammerhead, NCH (Inozyme), G-cleaver, amberzyme, zinzyme and/or DNAzyme motif, to inhibit the expression of HCV RNA.

In another embodiment, the invention features the use of an enzymatic nucleic acid molecule, preferably in the hammerhead, Inozyme, G-cleaver, amberzyme, zinzyme and/or DNAzyme motif, to inhibit the expression of HCV minus strand RNA.

In yet another embodiment, the invention features the use of a nuclease activating compound and/or a chimera to inhibit the expression of HCV RNA.

In another embodiment, the invention features the use of a nuclease activating compound and/or a chimera to inhibit the expression of HCVminus strand RNA.

By “inhibit” it is meant that the activity of HCV or level of RNAs or equivalent RNAs encoding one or more protein subunits of HCV is reduced below that observed in the absence of the nucleic acid molecules, nuclease activating compounds, and chimeras of the invention. In one embodiment, inhibition with an enzymatic nucleic acid molecule is below that level observed in the presence of an enzymatically inactive or attenuated molecule that is able to bind to the same site on the target RNA, but is unable to cleave that RNA. In another embodiment, inhibition of HCV genes with the nucleic acid molecule, nuclease activating compound, or chimera of the instant invention is greater than in the presence of the nucleic acid molecule, nuclease activating compound, or chimera than in its absence.

In one embodiment, the invention features a compound having formula I:

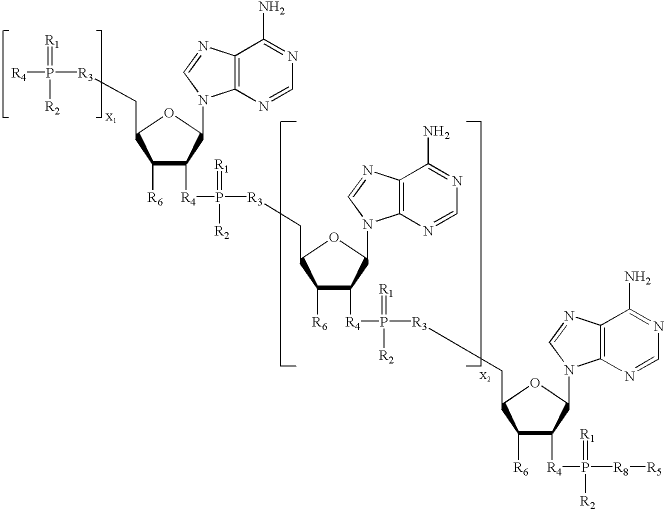

wherein X₁ is an integer of 1, 2, or 3; X₂ is an integer greater than or equal to 1; R₆ independently represents a 3′-ribofuranose sugar moiety, for example, H, OH, NH₂, O NH₂, alkyl, S-alkyl, O-alkyl, O-alkyl-S-alkyl, O-alkoxyalkyl, allyl, O-allyl, or fluoro; each R₁ and R₂ independently represent non-bridging phosphate moiety, for example, O, alkyl, O-alkyl, or S; each R₃ R₄ and R₈ independently represent a bridging phosphate moiety, for example, O, N, alkyl, fluoroalkyl, or S; and R₅ represents an alkyl or alkylamine group, or an oligonucleotide comprising any of SEQ ID NOS. 4798-9637, an oligonucleotide having a sequence complementary to a sequence comprising SEQ ID NOS. 1-4556, or abasic moiety.

In another embodiment, the abasic moiety of the instant invention preferably includes:

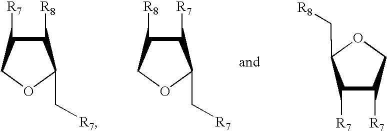

wherein R₈ is R₈ shown in Formula I and R₇ independently represents a ribofuranose sugar moiety, for example, H, OH, NH2, O—NH2, alkyl, S-alkyl, O-alkyl, O-alkyl-S-alkyl, O-alkoxyalkyl, allyl, O-allyl, fluoro, oligonucleotide, alkyl, alkylamine or abasic moiety.

In another embodiment, the oligonucleotide R₅ of Formula I having a sequence complementary to a sequence comprising SEQ ID NOs. 1-4556 is an enzymatic nucleic acid molecule.

In yet another embodiment, the oligonucleotide R₅ of Formula I having a sequence complementary to a sequence comprising SEQ ID NOs. 1-4556 is an antisense nucleic acid molecule.

In another embodiment, the oligonucleotide R₅ of Formula I having a sequence complementary to a sequence comprising SEQ ID NOs. 1-4556 is an enzymatic nucleic acid molecule selected from the group consisting of Hammerhead, Inozyme, G-cleaver, DNAzyme, Amberzyme, and Zinzyme motifs.

In another embodiment, the Inozyme enzymatic nucleic acid molecule of the instant invention comprises a stem II region of length greater than or equal to 2 base pairs.

In one embodiment, the oligonucleotide R₅ of Formula I having a sequence complementary to a sequence comprising SEQ ID NOs. 1-4556 is an enzymatic nucleic acid comprising between 12 and 100 bases complementary to an RNA derived from HCV.

In another embodiment, the oligonucleotide R₅ of Formula I having a sequence complementary to a sequence comprising SEQ ID NOs. 1-4556 is an enzymatic nucleic acid comprising between 14 and 24 bases complementary to said RNA derived from HCV.

In one embodiment, the oligonucleotide R₅ of Formula I having a sequence complementary to a sequence comprising SEQ ID NOs. 1-4556 is an antisense nucleic acid comprising between 12 and 100 bases complementary to an RNA derived from HCV.

In another embodiment, the oligonucleotide R₅ of Formula I having a sequence complementary to a sequence comprising SEQ ID NOs. 1-4556 is an antisense nucleic acid comprising between 14 and 24 bases complementary to said RNA derived from HCV.

In another embodiment, the invention features a pharmaceutical composition comprising a compound of Formula I, in a pharmaceutically acceptable carrier.

In yet another embodiment, the invention features a mammalian cell comprising a compound of Formula I. For example, the mammalian cell comprising a compound of Formula I is a human cell.

In one embodiment, the invention features a method for treatment of cirrhosis, liver failure or hepatocellular carcinoma comprising the step of administering to a patient a compound of Formula I under conditions suitable for said treatment.

In another embodiment, the invention features a method of treatment of a patient having a condition associated with HCV infection comprising contacting cells of said patient with a compound having Formula I, and further comprising contacting the cells with one or more other therapeutic compounds under conditions suitable for said treatment. Other therapeutic compounds include, for example, type I interferon, interferon alpha, interferon beta, consensus interferon, polyethylene glycol interferon, polyethylene glycol interferon alpha 2a, polyethylene glycol interferon alpha 2b, polyethylene glycol consensus interferon, treatment with an enzymatic nucleic acid molecule, and treatment with an antisense molecule.

In one embodiment of the inventive method, the other therapeutic compounds, for example, type I interferon, interferon alpha, interferon beta, consensus interferon, polyethylene glycol interferon, polyethylene glycol interferon alpha 2a, polyethylene glycol interferon alpha 2b, polyethylene glycol consensus interferon, treatment with an enzymatic nucleic acid molecule, and treatment with an antisense nucleic acid molecule, and the compound having Formula I are administered separately in separate pharmaceutically acceptable carriers.

In another embodiment, the other therapeutic compounds, for example, type I interferon, interferon alpha, interferon beta, consensus interferon, polyethylene glycol interferon, polyethylene glycol interferon alpha 2a, polyethylene glycol interferon alpha 2b, polyethylene glycol consensus interferon, treatment with an enzymatic nucleic acid molecule, and treatment with an antisense nucleic acid molecule, and the compound having Formula I are administered simultaneously in a pharmaceutically acceptable carrier.

In yet another embodiment, the invention features a method for inhibiting HCV replication in a mammalian cell comprising the step of administering to said cell a compound having Formula I under conditions suitable for said inhibition.

In another embodiment, the invention features a method of cleaving a separate RNA molecule comprising, contacting a compound having Formula I with the separate RNA molecule under conditions suitable for the cleavage of the separate RNA molecule. In one example, the method of cleaving a separate RNA molecule is carried out in the presence of a divalent cation, for example Mg2+.

In yet another embodiment, the method of cleaving a separate RNA molecule of the invention is carried out in the presence of a protein nuclease, for example, RNAse L.

In one embodiment, a compound having Formula I is chemically synthesized. Additionally, a compound having Formula I comprises at least one 2′-sugar modification, at least one nucleic acid base modification, and/or at least one phosphate modification.

By “enzymatic nucleic acid molecule” it is meant a nucleic acid molecule which has complementarity in a substrate binding region to a specified gene target, and also has an enzymatic activity which is active to specifically cleave target RNA. That is, the enzymatic nucleic acid molecule is able to intermolecularly cleave RNA and thereby inactivate a target RNA molecule. These complementary regions allow sufficient hybridization of the enzymatic nucleic acid molecule to the target RNA and thus permit cleavage. One hundred percent complementarity is preferred, but complementarity as low as 50-75% can also be useful in this invention. The nucleic acids can be modified at the base, sugar, and/or phosphate groups. The term enzymatic nucleic acid is used interchangeably with phrases such as enzymatic nucleic acids, catalytic RNA, enzymatic RNA, catalytic DNA, aptazyme or aptamer-binding enzymatic nucleic acid, regulatable enzymatic nucleic acid, allosteric catalytic nucleic acid, allosteric enzymatic nucleic acid, allosteric ribozyme, catalytic oligonucleotides, nucleozyme, DNAzyme, RNA enzyme, endoribonuclease, endonuclease, minizyme, leadzyme, oligozyme or DNA enzyme. All of these terminologies describe nucleic acid molecules with enzymatic activity. The specific enzymatic nucleic acid molecules described in the instant application are not meant to be limiting and those skilled in the art will recognize that all that is important in an enzymatic nucleic acid molecule of this invention is that it have a specific substrate binding site which is complementary to one or more of the target nucleic acid regions, and that it have nucleotide sequences within or surrounding that substrate binding site which impart a nucleic acid cleaving activity to the molecule (Cech et al., U.S. Pat. No. 4,987,071; Cech et al., 1988, JAMA).

By “nucleic acid molecule” as used herein is meant a molecule having nucleotides. The nucleic acid can be single, double, or multiple stranded and can comprise modified or unmodified nucleotides or non-nucleotides or various mixtures and combinations thereof.

By “nuclease activating compound” is meant a compound, for example a compound having Formula I, that activates the cleavage of an RNA by a nuclease. The nuclease can comprise RNAse L. By “nuclease activating chimera” or “chimera” is meant a nuclease activating compound, for example a compound having Formula I, that is attached to a nucleic acid molecule, for example a nucleic acid molecule that binds preferentially to a target RNA. These chimeric nucleic acid molecules can comprise a nuclease activating compound and an antisense nucleic acid molecule, for example a 2′,5′-oligoadenylate antisense chimera, or an enzymatic nucleic acid molecule, for example a 2′,5′-oligoadenylate enzymatic nucleic acid chimera.

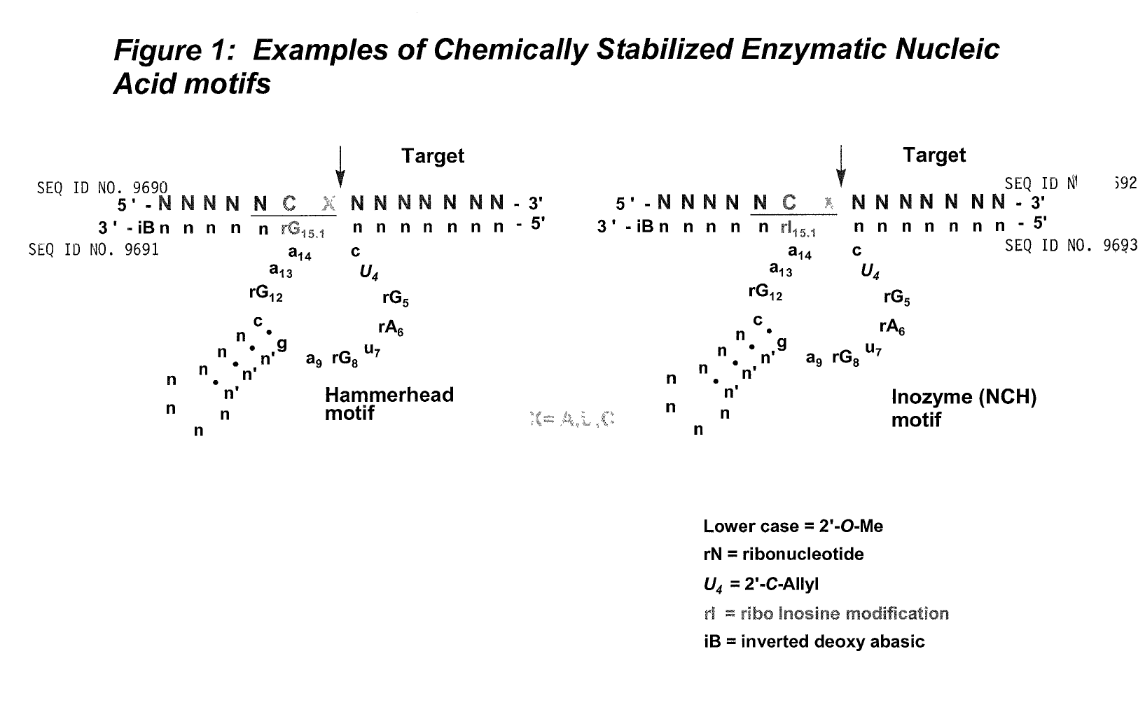

By “enzymatic portion” or “catalytic domain” is meant that portion/region of the enzymatic nucleic acid essential for cleavage of a nucleic acid substrate (for example, see FIG. 1).

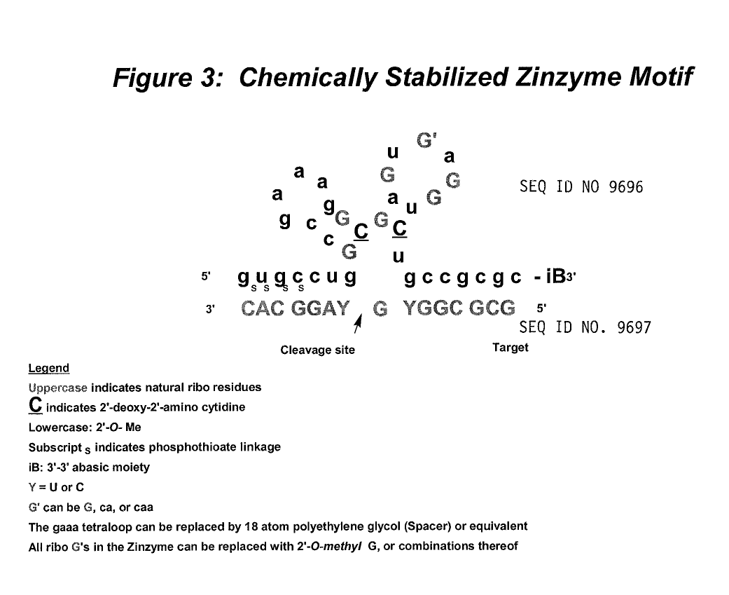

By “substrate binding arm” or “substrate binding domain” is meant that portion/region of an enzymatic nucleic acid which is complementary to (i.e., able to base-pair with) a portion of its substrate. Generally, such complementarity is 100%, but can be less if desired. For example, as few as 10 bases out of 14 can be base-paired. Such arms are shown generally in FIGS. 1 and 3. That is, these arms contain sequences within an enzymatic nucleic acid which are intended to bring enzymatic nucleic acid and target RNA together through complementary base-pairing interactions. The enzymatic nucleic acid of the invention can have binding arms that are contiguous or non-contiguous and can be of varying lengths. The length of the binding arm(s) are preferably greater than or equal to four nucleotides; specifically 12-100 nucleotides; more specifically 14-24 nucleotides long. If two binding arms are chosen, the design is such that the length of the binding arms are symmetrical (i.e., each of the binding arms is of the same length; e.g., five and five nucleotides, six and six nucleotides or seven and seven nucleotides long) or asymmetrical (i.e., the binding arms are of different length; e.g., six and three nucleotides; three and six nucleotides long; four and five nucleotides long; four and six nucleotides long; four and seven nucleotides long; and the like).

By “Inozyme” or “NCH” motif is meant an enzymatic nucleic acid molecule comprising a motif as is generally described in FIG. 1 and in Ludwig et al., International PCT publication Nos. WO 98/58058 and WO 98/58057. Inozymes possess endonuclease activity to cleave RNA substrates having a cleavage triplet NCH/, where N is a nucleotide, C is cytidine and H is adenosine, uridine or cytidine, and/represents the cleavage site. H is used interchangeably with X. Inozymes can also possess endonuclease activity to cleave RNA substrates having a cleavage triplet NCN/, where N is a nucleotide, C is cytidine, and/represents the cleavage site. “I” in FIG. 1 represents an Inosine nucleotide, preferably a ribo-Inosine or xylo-Inosine nucleotide.

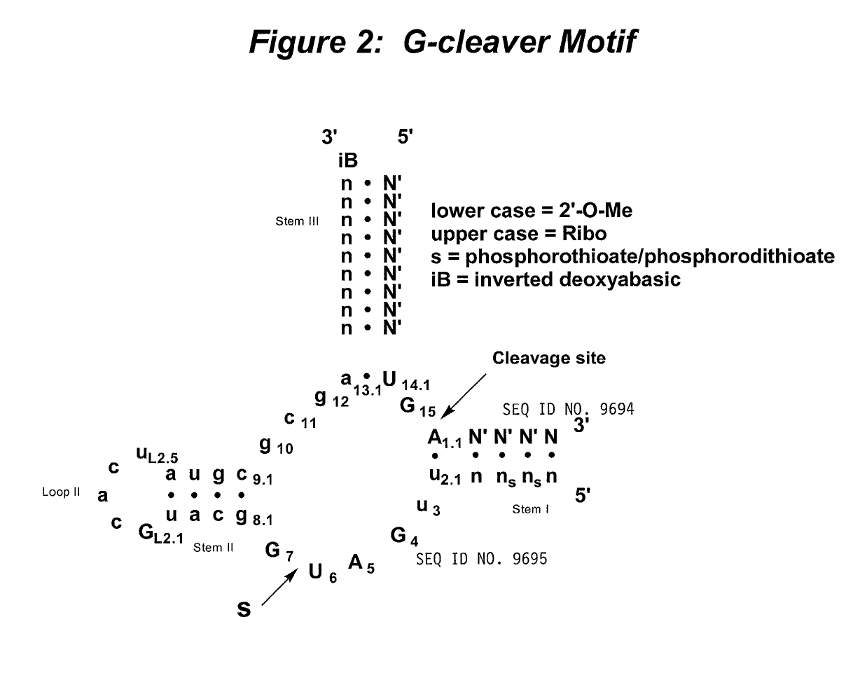

By “G-cleaver” motif is meant an enzymatic nucleic acid molecule comprising a motif as is generally described in FIG. 2 and in Eckstein et al., International PCT publication No. WO/9916871. G-cleavers possess endonuclease activity to cleave RNA substrates having a cleavage triplet NYN/, where N is a nucleotide, Y is uridine or cytidine and/represents the cleavage site. G-cleavers can be chemically modified as is generally shown in FIG. 2. G-cleavers can be used, for example, to cleave RNA substrates after an AUG/triplet, where A is adenosine, U is uridine, G is guanosine, and/represents the cleavage site.

By “zinzyme” motif is meant an enzymatic nucleic acid molecule comprising a motif as is generally described in FIG. 3 and in Beigelman et al, International PCT publication No. WO/9955857. Zinzymes possess endonuclease activity to cleave RNA substrates having a cleavage triplet including but not limited to YG/Y, where Y is uridine or cytidine, and G is guanosine and/represents the cleavage site. Zinzymes can be chemically modified to increase nuclease stability through chemical modifications or substitutions as generally shown in FIG. 3, including substituting 2′-O-methyl guanosine nucleotides for guanosine nucleotides. In addition, differing nucleotide and/or non-nucleotide linkers can be used to substitute the 5′-gaaa-2′ loop shown in the figure. Zinzymes represent a non-limiting example of an enzymatic nucleic acid molecule that does not require a ribonucleotide (2′-OH) group within its own nucleic acid sequence for activity.

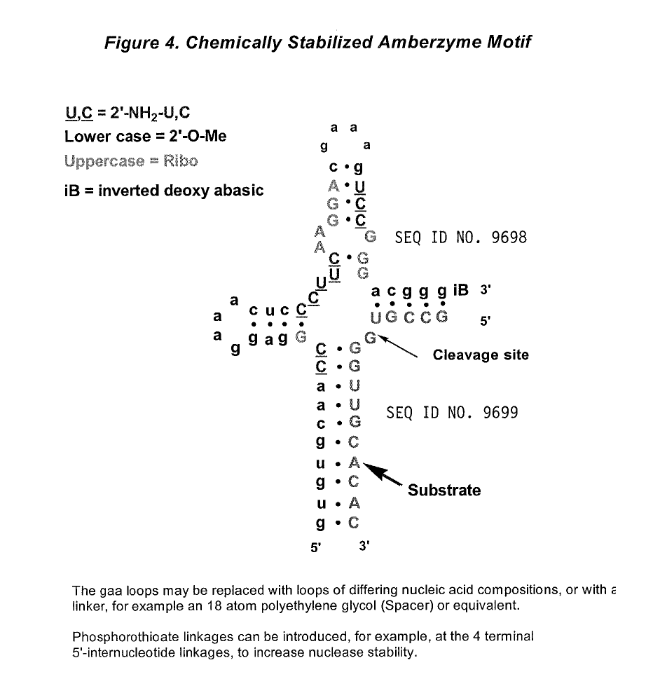

By “amberzyme” motif is meant an enzymatic nucleic acid molecule comprising a motif as is generally described in FIG. 4 and in Beigelman et al., International PCT publication No. WO/9955857. Amberzymes possess endonuclease activity to cleave RNA substrates having a cleavage triplet NG/N, where N is a nucleotide, G is guanosine, and/represents the cleavage site. Amberzymes can be chemically modified to increase nuclease stability through substitutions as are generally shown in FIG. 4. In addition, differing nucleoside and/or non-nucleoside linkers can be used to substitute the 5′-gaaa-3′ loops shown in the figure. Amberzymes represent a non-limiting example of an enzymatic nucleic acid molecule that does not require a ribonucleotide (2′-OH) group within its own nucleic acid sequence for activity.

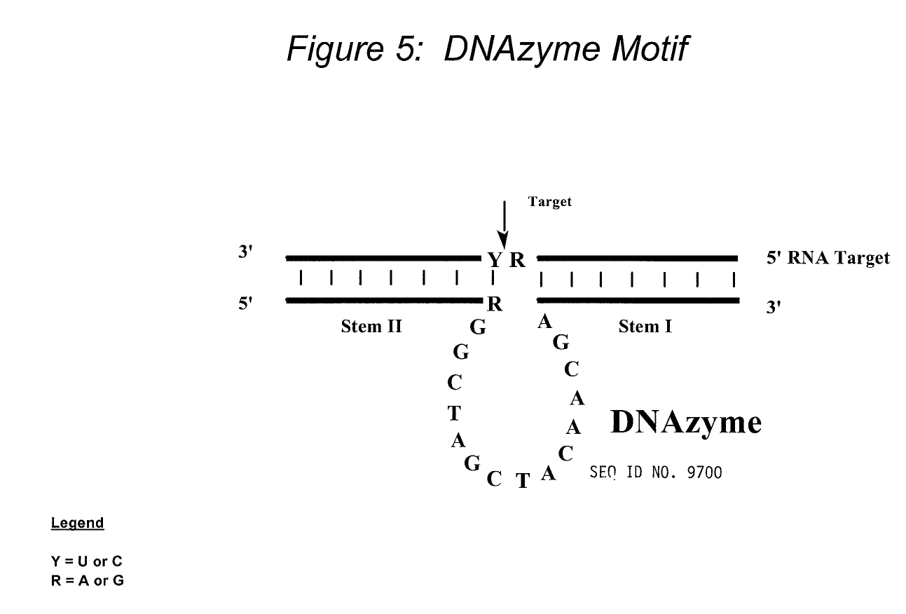

By ‘DNAzyme’ is meant an enzymatic nucleic acid molecule that does not require the presence of a 2′-OH group for its activity. In particular embodiments the enzymatic nucleic acid molecule can have an attached linker(s) or other attached or associated groups, moieties, or chains containing one or more nucleotides with 2′-OH groups. DNAzymes can be synthesized chemically or expressed endogenously in vivo, by means of a single stranded DNA vector or equivalent thereof. An example of a DNAzyme is shown in FIG. 5 and is generally reviewed in Usman et al., International PCT Publication No. WO 95/11304; Chartrand et al., 1995, NAR 23, 4092; Breaker et al., 1995, Chem. Bio. 2, 655; Santoro et al., 1997, PNAS 94, 4262; Breaker, 1999, Nature Biotechnology, 17, 422-423; and Santoro et. al., 2000, J. Am. Chem. Soc., 122, 2433-39. Additional DNAzyme motifs can be selected by using techniques similar to those described in these references, and hence, are within the scope of the present invention.

By “antisense nucleic acid”, it is meant a non-enzymatic nucleic acid molecule that binds to target RNA by means of RNA-RNA or RNA-DNA or RNA-PNA (protein nucleic acid; Egholm et al., 1993 Nature 365, 566) interactions and alters the activity of the target RNA (for a review, see Stein and Cheng, 1993 Science 261, 1004 and Woolf et al., U.S. Pat. No. 5,849,902). Typically, antisense molecules are complementary to a target sequence along a single contiguous sequence of the antisense molecule. However, in certain embodiments, an antisense molecule can bind to substrate such that the substrate molecule forms a loop, and/or an antisense molecule can bind such that the antisense molecule forms a loop. Thus, the antisense molecule can be complementary to two (or even more) non-contiguous substrate sequences or two (or even more) non-contiguous sequence portions of an antisense molecule can be complementary to a target sequence or both. For a review of current antisense strategies, see Schmajuk et al., 1999, J. Biol. Chem., 274, 21783-21789, Delihas et al., 1997, Nature, 15, 751-753, Stein et al., 1997, Antisense N. A. Drug Dev., 7, 151, Crooke, 2000, Methods Enzymol., 313, 3-45; Crooke, 1998, Biotech. Genet. Eng. Rev., 15, 121-157, Crooke, 1997, Ad. Pharmacol., 40, 1-49. In addition, antisense DNA can be used to target RNA by means of DNA-RNA interactions, thereby activating RNase H, which digests the target RNA in the duplex. The antisense oligonucleotides can comprise one or more RNAse H activating region, which is capable of activating RNAse H cleavage of a target RNA. Antisense DNA can be synthesized chemically or expressed via the use of a single stranded DNA expression vector or equivalent thereof.

By “RNase H activating region” is meant a region (generally greater than or equal to 4-25 nucleotides in length, preferably from 5-11 nucleotides in length) of a nucleic acid molecule capable of binding to a target RNA to form a non-covalent complex that is recognized by cellular RNase H enzyme (see for example Arrow et al., U.S. Pat. No. 5,849,902; Arrow et al., U.S. Pat. No. 5,989,912). The RNase H enzyme binds to the nucleic acid molecule-target RNA complex and cleaves the target RNA sequence. The RNase H activating region comprises, for example, phosphodiester, phosphorothiote (preferably at least four of the nucleotides are phosphorothiote substitutions; more specifically, 4-11 of the nucleotides are phosphorothiote substitutions); phosphorodithioate, 5′-thiophosphate, or methylphosphonate backbone chemistry or a combination thereof. In addition to one or more backbone chemistries described above, the RNase H activating region can also comprise a variety of sugar chemistries. For example, the RNase H activating region can comprise deoxyribose, arabino, fluoroarabino or a combination thereof, nucleotide sugar chemistry. Those skilled in the art will recognize that the foregoing are non-limiting examples and that any combination of phosphate, sugar and base chemistry of a nucleic acid that supports the activity of RNase H enzyme is within the scope of the definition of the RNase H activating region and the instant invention.

By “2-5A antisense” or “2-5A antisense chimera” is meant an antisense oligonucleotide containing a 5′-phosphorylated 2′-5′-linked adenylate residue. These chimeras bind to target RNA in a sequence-specific manner and activate a cellular 2-5A-dependent ribonuclease which, in turn, cleaves the target RNA (Torrence et al., 1993 Proc. Natl. Acad. Sci. USA 90, 1300; Silverman et al., 2000, Methods Enzymol., 313, 522-533; Player and Torrence, 1998, Pharmacol. Ther., 78, 55-113).

By “sufficient length” is meant an oligonucleotide of greater than or equal to 3 nucleotides.

By “stably interact” is meant, interaction of the oligonucleotides with target nucleic acid (e.g., by forming hydrogen bonds with complementary nucleotides in the target under physiological conditions).

By “equivalent” RNA to HCV is meant to include those naturally occurring RNA molecules associated with HCV infection in various animals, including human, rodent, primate, rabbit and pig. The equivalent RNA sequence also includes in addition to the coding region, regions such as 5′-untranslated region, 3′-untranslated region, introns, intron-exon junction and the like.

By “homology” is meant the nucleotide sequence of two or more nucleic acid molecules is partially or completely identical.

In one of the preferred embodiments of the inventions described herein, the enzymatic nucleic acid molecule is formed in a hammerhead or hairpin motif, but can also be formed in the motif of a hepatitis delta virus, group I intron, group II intron or RNase P RNA (in association with an RNA guide sequence), DNAzymes, NCH cleaving motifs (inozymes), or G-cleavers. Examples of such hammerhead motifs (FIG. 1a) are described in Dreyfus, supra, Rossi et al., 1992, AIDS Research and Human Retroviruses 8, 183; Examples of hairpin motifs are described in Hampel et al., EP0360257, Hampel and Tritz, 1989 Biochemistry 28, 4929, Feldstein et al., 1989, Gene 82, 53, Haseloff and Gerlach, 1989, Gene, 82, 43, Hampel et al., 1990 Nucleic Acids Res. 18, 299; Chowrira & McSwiggen, U.S. Pat. No. 5,631,359. The hepatitis delta virus motif is generally described in Perrotta and Been, 1992 Biochemistry 31, 16. The RNase P motif is generally described in Guerrier-Takada et al., 1983 Cell 35, 849; Forster and Altman, 1990, Science 249, 783; Li and Altman, 1996, Nucleic Acids Res. 24, 835. Examples of group II introns are generally described in Griffin et al., 1995, Chem. Biol. 2, 761; Michels and Pyle, 1995, Biochemistry 34, 2965; Pyle et al, International PCT Publication No. WO 96/22689. The Group I intron is generally described in Cech et al., U.S. Pat. No. 4,987,071. DNAzymes (FIG. 4) are generally described in Usman et al., International PCT Publication No. WO 95/11304; Chartrand et al., 1995, NAR 23, 4092; Breaker et al., 1995, Chem. Bio. 2, 655; Santoro et al., 1997, PNAS 94, 4262; Breaker, 1999, Nature Biotechnology, 17, 422-423; Santoro et. al., 2000, J. Am. Chem. Soc., 122, 2433-39). NCH cleaving motifs (FIG. 1b) are generally described in Ludwig & Sproat, International PCT Publication No. WO 98/58058; and G-cleavers (FIG. 1c) are generally described in Kore et al., 1998, Nucleic Acids Research 26, 4116-4120 and Eckstein et al., International PCT Publication No. WO 99/16871. Additional motifs contemplated by the instant invention include the Allozyme or allosteric enzymatic nucleic acid molecule (Breaker et al., WO 98/43993, Shih et. al., U.S. Pat. No. 5,589,332, George et al., U.S. Pat. No. 5,741,679), Amberzyme (FIG. 2, Class I motif in Beigelman et al., International PCT publication No. WO 99/55857) and Zinzyme (FIG. 3, Class II motif in Beigelman et al, International PCT publication No. WO 99/55857), all these references are incorporated by reference herein in their totalities, including drawings and can also be used in the present invention. These specific motifs are not limiting in the invention. Those skilled in the art will recognize that all that is important is that the enzymatic molecule have a specific substrate binding site which is complementary to one or more of the target gene RNA regions, and that it have nucleotide sequences within or surrounding that substrate binding site which impart an RNA cleaving activity to the molecule (Cech et al., U.S. Pat. No. 4,987,071).

By “complementarity” is meant that a nucleic acid can form hydrogen bond(s) with another RNA sequence by either traditional Watson-Crick or other non-traditional types. In reference to the nucleic molecules of the present invention, the binding free energy for a nucleic acid molecule with its target or complementary sequence is sufficient to allow the relevant function of the nucleic acid to proceed, e.g., enzymatic nucleic acid cleavage. Determination of binding free energies for nucleic acid molecules is well known in the art (see, e.g., Turner et al., 1987, CSH Symp. Quant. Biol. LII pp.123-133; Frier et al., 1986, Proc. Nat. Acad. Sci. USA 83:9373-9377; Turner et al., 1987, J. Am. Chem. Soc. 109:3783-3785). A percent complementarity indicates the percentage of contiguous residues in a nucleic acid molecule which can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary). “Perfectly complementary” means that all the contiguous residues of a nucleic acid sequence will hydrogen bond with the same number of contiguous residues in a second nucleic acid sequence.

In a preferred embodiment, the invention provides a method for producing a class of enzymatic cleaving agents which exhibit a high degree of specificity for the RNA of a desired target. The enzymatic nucleic acid molecule, nuclease activating compound or chimera is preferably targeted to a highly conserved sequence region of a target mRNAs encoding HCV proteins such that specific treatment of a disease or condition can be provided with either one or several enzymatic nucleic acids. Such nucleic acid molecules can be delivered exogenously to specific cells as required. Alternatively, the enzymatic nucleic acid molecules can be expressed from DNA/RNA vectors that are delivered to specific cells. DNAzymes can be synthesized chemically or expressed endogenously in vivo, by means of a single stranded DNA vector or equivalent thereof.

By “highly conserved sequence region” is meant a nucleotide sequence of one or more regions in a target gene does not vary significantly from one generation to the other or from one biological system to the other.

Such enzymatic nucleic acid molecules, nuclease activating compound or chimera molecules are useful for the prevention of the diseases and conditions discussed above, and any other diseases or conditions that are related to the levels of HCV activity in a cell or tissue.

By “related” is meant that the inhibition of HCV RNAs and thus reduction in the level respective viral activity will relieve to some extent the symptoms of the disease or condition.

The nucleic acid-based inhibitors, nuclease activating compounds and chimeras of the invention are added directly, or can be complexed with cationic lipids, packaged within liposomes, or otherwise delivered to target cells or tissues. The nucleic acid or nucleic acid complexes, and nuclease activating compounds or chimeras can be locally administered to relevant tissues ex vivo, or in vivo through injection or infusion pump, with or without their incorporation in biopolymers. In preferred embodiments, the enzymatic nucleic acid inhibitors, and nuclease activating compounds or chimeras comprise sequences, which are complementary to the substrate sequences in Tables III, IV, V and VIII. Examples of such enzymatic nucleic acid molecules also are shown in Tables III, IV, V, VI and VIII. Examples of such enzymatic nucleic acid molecules consist essentially of sequences defined in these tables. In additional embodiments, the enzymatic nucleic acid inhibitors of the invention that comprise sequences which are complementary to the substrate sequences in Tables III, IV, V and VIII are covalently attached to nuclease activating compound or chimeras of the invention, for example a compound having Formula I.

In yet another embodiment, the invention features antisense nucleic acid molecules and 2-5A chimera including sequences complementary to the substrate sequences shown in Tables III, IV, V and VIII. Such nucleic acid molecules can include sequences as shown for the binding arms of the enzymatic nucleic acid molecules in Tables III, IV, V, VI and VIII. Similarly, triplex molecules can be provided targeted to the corresponding DNA target regions, and containing the DNA equivalent of a target sequence or a sequence complementary to the specified target (substrate) sequence. Typically, antisense molecules are complementary to a target sequence along a single contiguous sequence of the antisense molecule. However, in certain embodiments, an antisense molecule can bind to substrate such that the substrate molecule forms a loop, and/or an antisense molecule can bind such that the antisense molecule forms a loop. Thus, the antisense molecule can be complementary to two (or even more) non-contiguous substrate sequences or two (or even more) non-contiguous sequence portions of an antisense molecule can be complementary to a target sequence or both.

By “consists essentially of” is meant that the active compound or nucleic acid molecule of the invention, for example an enzymatic nucleic acid molecule, contains an enzymatic center or core equivalent to those in the examples, and binding arms able to bind RNA such that cleavage at the target site occurs. Other sequences can be present which do not interfere with such cleavage. Thus, a core region can, for example, include such a loop, stem-loop, nucleotide linker, and/or non-nucleotide linker and can be represented generally as sequence “X”. For example, a core sequence for a hammerhead enzymatic nucleic acid can comprise a conserved sequence, such as 5′-CUGAUGAG-3′ and 5′-CGAA-3′ connected by “X”, where X is 5′-GCCGUUAGGC-3′ (SEQ ID NO. 9704), or any other Stem II region known in the art, or a nucleotide and/or non-nucleotide linker. Similarly, for other compounds and nucleic acid molecules of the instant invention, such as Inozyme, G-cleaver, amberzyme, zinzyme, DNAzyme, antisense, and 2-5A antisense, other sequences or non-nucleotide linkers can be present that do not interfere with the function of the nucleic acid molecule.

Sequence X can be a linker of ≧2 nucleotides in length, preferably 3, 4, 5, 6, 7, 8, 9, 10, 15, 20, 26, 30, where the nucleotides can preferably be internally base-paired to form a stem of preferably ≧2 base pairs. Alternatively or in addition, sequence X can be a non-nucleotide linker. In yet another embodiment, the nucleotide linker X can be a nucleic acid aptamer, such as an ATP aptamer, HIV Rev aptamer (RRE), HIV Tat aptamer (TAR) and others (for a review see Gold et al., 1995, Annu. Rev. Biochem., 64, 763; and Szostak & Ellington, 1993, in The RNA World, ed. Gesteland and Atkins, pp. 511, CSH Laboratory Press). A “nucleic acid aptamer” as used herein is meant to indicate a nucleic acid sequence capable of interacting with a ligand. The ligand can be any natural or a synthetic molecule, including but not limited to a resin, metabolites, nucleosides, nucleotides, drugs, toxins, transition state analogs, peptides, lipids, proteins, amino acids, nucleic acid molecules, hormones, carbohydrates, receptors, cells, viruses, bacteria and others.

In yet another embodiment, the non-nucleotide linker X is as defined herein. The term “non-nucleotide” as used herein include either abasic nucleotide, polyether, polyamine, polyamide, peptide, carbohydrate, lipid, or polyhydrocarbon compounds. Specific examples include those described by Seela and Kaiser, Nucleic Acids Res. 1990, 18:6353 and Nucleic Acids Res. 1987, 15:3113; Cload and Schepartz, J. Am. Chem. Soc. 1991, 113:6324; Richardson and Schepartz, J. Am. Chem. Soc. 1991, 113:5109; Ma et al., Nucleic Acids Res. 1993, 21:2585 and Biochemistry 1993, 32:1751; Durand et al., Nucleic Acids Res. 1990, 18:6353; McCurdy et al., Nucleosides & Nucleotides 1991, 10:287; Jschke et al., Tetrahedron Lett. 1993, 34:301; Ono et al., Biochemistry 1991, 30:9914; Arnold et al., International Publication No. WO 89/02439; Usman et al., International Publication No. WO 95/06731; Dudycz et al., International Publication No. WO 95/11910 and Ferentz and Verdine, J. Am. Chem. Soc. 1991, 113:4000, all hereby incorporated by reference herein. A “non-nucleotide” further means any group or compound which can be incorporated into a nucleic acid chain in the place of one or more nucleotide units, including either sugar and/or phosphate substitutions, and allows the remaining bases to exhibit their enzymatic activity. The group or compound can be abasic in that it does not contain a commonly recognized nucleotide base, such as adenosine, guanine, cytosine, uracil or thymine. Thus, in a preferred embodiment, the invention features an enzymatic nucleic acid molecule having one or more non-nucleotide moieties, and having enzymatic activity to cleave an RNA or DNA molecule.

Thus, in a first aspect, the invention features nucleic acid molecules and nuclease activating compounds or chimeras that inhibit gene expression and/or viral replication. These chemically or enzymatically synthesized nucleic acid molecules can contain substrate binding domains that bind to accessible regions of their target mRNAs. The nucleic acid molecules also contain domains that catalyze the cleavage of RNA. The enzymatic nucleic acid molecules are preferably molecules of the hammerhead, Inozyme, DNAzyme, Zinzyme, Amberzyme, and/or G-cleaver motifs. Upon binding, the enzymatic nucleic acid molecules cleave the target mRNAs, preventing translation and protein accumulation. In the absence of the expression of the target gene, HCV gene expression and/or replication is inhibited.

In one embodiment, the nucleic acid molecules and nuclease activating compounds or chimeras are added directly, or can be complexed with cationic lipids, packaged within liposomes, or otherwise delivered to target cells using delivery methods described herein and known in the art. The nucleic acid or nucleic acid complexes can be locally administered to relevant tissues ex vivo, or in vivo through injection, infusion pump or stent, with or without their incorporation in biopolymers. In another embodiment, the nucleic acid molecule, nuclease activating compound or chimera is administered to the site of HCV activity (e.g., hepatocytes) in an appropriate liposomal vehicle.

In another embodiment of the invention, nucleic acid molecules that cleave target molecules and inhibit HCV activity are expressed from transcription units inserted into DNA or RNA vectors. The recombinant vectors are preferably DNA plasmids or viral vectors. Nucleic acid molecule expressing viral vectors can be constructed based on, but not limited to, adeno-associated virus, retrovirus, adenovirus, or alphavirus. Preferably, the recombinant vectors capable of expressing the nucleic acid molecules are delivered as described above, and persist in target cells. Alternatively, viral vectors can be used that provide for transient expression of nucleic acid molecules. Such vectors can be repeatedly administered as necessary. Once expressed, the nucleic acid molecules cleave the target mRNA. Delivery of enzymatic nucleic acid molecule expressing vectors can be systemic, such as by intravenous or intramuscular administration, by administration to target cells ex-planted from the patient followed by reintroduction into the patient, or by any other means that would allow for introduction into the desired target cell (for a review see Couture and Stinchcomb, 1996, TIG., 12, 510). In another aspect of the invention, nucleic acid molecules that cleave target molecules and inhibit viral replication are expressed from transcription units inserted into DNA, RNA, or viral vectors. Preferably, the recombinant vectors capable of expressing the nucleic acid molecules are locally delivered as described above, and transiently persist in smooth muscle cells. However, other mammalian cell vectors that direct the expression of RNA can be used for this purpose.

By “patient” is meant an organism which is a donor or recipient of explanted cells or the cells themselves. “Patient” also refers to an organism to which enzymatic nucleic acid molecules can be administered. Preferably, a patient is a mammal or mammalian cells. More preferably, a patient is a human or human cells.

As used in herein “cell” is used in its usual biological sense, and does not refer to an entire multicellular organism, e.g., specifically does not refer to an entire human. The cell can be present in a multicellular organism, e.g., birds, plants and mammals such as cows, sheep, apes, monkeys, swine, dogs, and cats.

By RNA is meant a molecule comprising at least one ribonucleotide residue. By “ribonucleotide” is meant a nucleotide with a hydroxyl group at the 2′ position (eg; 2′-OH) of a β-D-ribo-furanose moiety.

By “vectors” is meant any nucleic acid- and/or viral-based technique used to deliver a desired nucleic acid.

These nucleic acid molecules, nuclease activating compounds and chimeras individually, or in combination or in conjunction with other drugs, can be used to treat diseases or conditions discussed above. For example, to treat a disease or condition associated with HCV levels, the patient can be treated, or other appropriate cells can be treated, as is evident to those skilled in the art.

In a further embodiment, the described molecules can be used in combination with other known treatments to treat conditions or diseases discussed above. For example, the described molecules could be used in combination with one or more known therapeutic agents to treat liver failure, hepatocellular carcinoma, cirrhosis, and/or other disease states associated with HCV infection. Additional known therapeutic agents are those comprising antivirals, interferons, and/or antisense compounds.

By “comprising” is meant including, but not limited to, whatever follows the word “comprising”. Thus, use of the term “comprising” indicates that the listed elements are required or mandatory, but that other elements are optional and can or can not be present. By “consisting of” is meant including, and limited to, whatever follows the phrase “consisting of”. Thus, the phrase “consisting of” indicates that the listed elements are required or mandatory, and that no other elements can be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and can or can not be present depending upon whether or not they affect the activity or action of the listed elements.

Other features and advantages of the invention will be apparent from the following description of the preferred embodiments thereof, and from the claims.

### DESCRIPTION OF THE PREFERRED EMBODIMENTS

The drawings will first briefly be described.

Drawings:

FIG. 1 is a diagrammatic representation of a Hammerhead and an Inozyme motif. The examples shown are chemically stabilized with 2′-O-methyl substitutions (lower case), a 2′-deoxy-2′-C-allyl Uridine substitution at position U-4, and a 3′-terminal inverted deoxyabasic moiety. Conserved ribonucleotides are shown as rN, for example G-5, A-6, G-8, G-2, and I-15.1. Phosphorothioate internucleotide substitutions can be introduced, for example, at the four terminal 5′ end nucleotides for increased stability to nuclease degradation. Stem II can be >2 base-pair long, preferably, 2, 3, 4, 5, 6, 7, 8, and 10 base-pairs long. Each N and N′ is independently any base or non-nucleotide as used herein; X is adenosine, cytidine or uridine; Stems I-III are meant to indicate three stem-loop structures; stems I-III can be of any length and can be symmetrical or asymmetrical; arrow indicates the site of cleavage in the target RNA; Rz refers to enzymatic nucleic acid; Loop II can be present or absent. If Loop II is present it is greater than or equal to three nucleotides, preferably four nucleotides. The Loop II sequence is preferably 5′-GAAA-3′ or 5′-GUUA-3′. Inozyme position 15.1 comprises an Inosine nucleotide, which can be ribo-Inosine or xylo-Inosine.

FIG. 2 is a diagrammatic representation of a G-cleaver motif. The example shown is chemically stabilized with 2′-O-methyl substitutions, phosphorothioate internucleotide linkage substitutions, and a 3′-termianl inverted deoxyabasic moiety. In the figure, lower case a, g, c, and u represent 2′-O-methyl adenosine, guanosine, cytidine, and uridine nucleotides respectively; upper case A, G, C and U represent adenosine, guanosine, cytidine and uridine nucleotides respectively; “s” refers to phosphorothioate internucleotide linkages, and iB represents an 3′-terminal inverted deoxyabasic moiety.

FIG. 3 is a diagrammatic representation of a zinzyme motif. The example shown is chemically stabilized with 2′-O-methyl substitutions, phosphorothioate internucleotide linkage substitutions, and a 3′-termianl inverted deoxyabasic moiety. C in the figure represents a 2′-deozy-2′-amino cytidine nucleotide; lower case a, g, c, and u represent 2′-O-methyl adenosine, guanosine, cytidine, and uridine nucleotides respectively; uppercase A, G, C and U represent adenosine, guanosine, cytidine and uridine nucleotides respectively; “s” refers to phosphorothioate internucleotide linkages, and iB represents an 3′-terminal inverted deoxyabasic moiety. All of the ribo-guanosine nucleotides in the zinzyme motif can be replaced with 2′-O-methyl guanosine nucleotides. The 5′-gaaa-3′ loop can be replaced with other nucleotide containing loop structures and/or non-nucleotide linkers, including PEG linkers. The guanosine nucleotide represented as G′ in the figure can be replaced with either 2′-O-methyl guanosine, 5′-cytidine-adenosine-3′, or 5′-cytidine-adenosine-adenosine-3′ nucleotides and/or their corresponding 2′-O-methyl nucleotide derivatives.

FIG. 4 is a diagrammatic representation of an amberzyme motif. The example shown is chemically stabilized with 2′-O-methyl substitutions and a 3′-termianl inverted deoxyabasic moiety. C in the figure represents a 2′-deoxy-2′-amino cytidine nucleotide; lower case a, g, c, and u represent 2′-O-methyl adenosine, guanosine, cytidine, and uridine nucleotides respectively; uppercase A, G, C and U represent adenosine, guanosine, cytidine and uridine nucleotides respectively; and iB represents an 3′-terminal inverted deoxyabasic moiety. The amberzyme motif can be further stabilized through introducing phosphorothioate internucleotide linkages, for example at the four terminal 5′-internucleotide linkages.

FIG. 5 is a diagrammatic representation of a DNAzyme motif described generally, for example in Santoro et al., 1997, PNAS, 94, 4262.

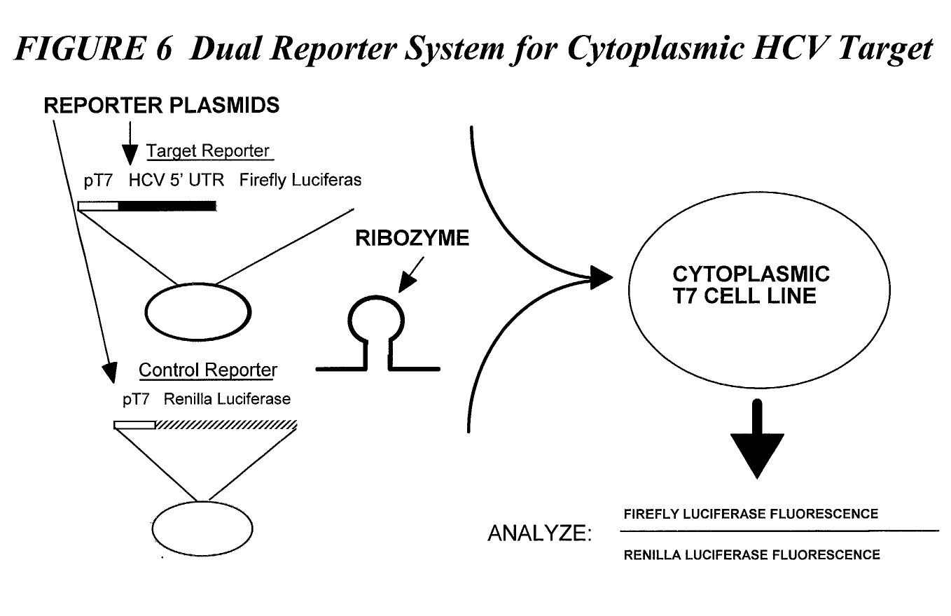

FIG. 6 is a schematic representation of the Dual Reporter System utilized to demonstrate enzymatic nucleic acid mediated reduction of luciferase activity in cell culture.

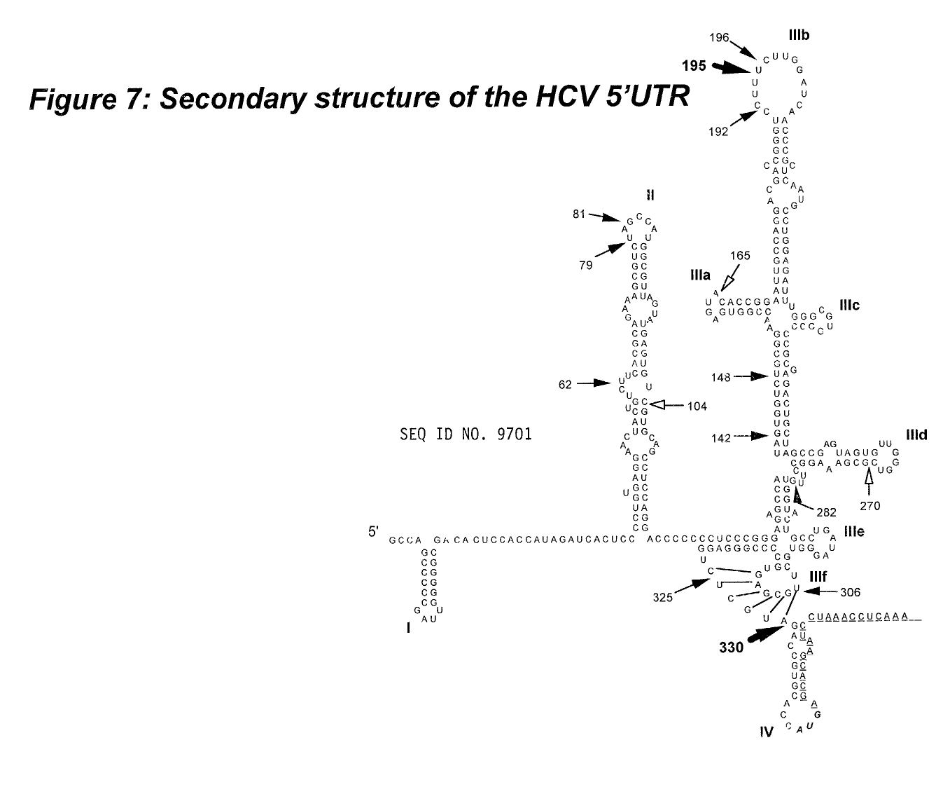

FIG. 7 shows a schematic view of the secondary structure of the HCV 5′UTR (Brown et al., 1992, Nucleic Acids Res., 20, 5041-45; Honda et al., 1999, J. Virol., 73, 1165-74). Major structural domains are indicated in bold. Enzymatic nucleic acid cleavage sites are indicated by arrows. Solid arrows denote sites amenable to amino-modified enzymatic nucleic acid inhibition. Lead cleavage sites (195 and 330) are indicated with oversized solid arrows.

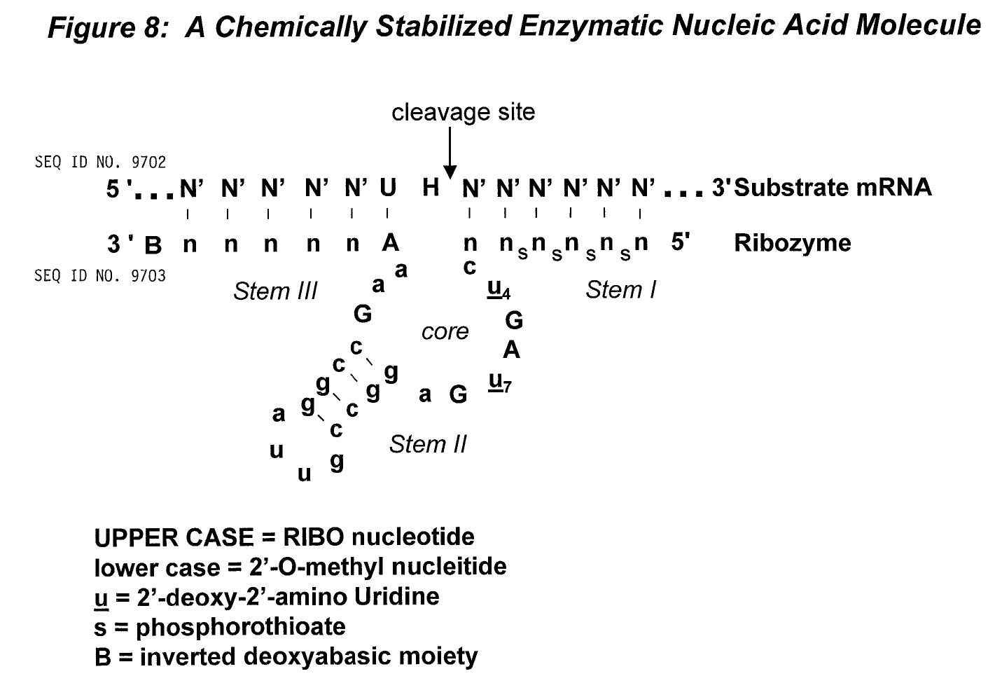

FIG. 8 shows a non-limiting example of a nuclease resistant enzymatic nucleic acid molecule. Binding arms are indicated as stem I and stem III. Nucleotide modifications are indicated as follows: 2′-O-methyl nucleotides, lowercase; ribonucleotides, uppercase G, A; 2′-amino-uridine, u; inverted 3′-3′ deoxyabasic, B. The positions of phosphorothioate linkages at the 5′-end of each enzymatic nucleic acid are indicated by subscript “s”. H indicates A, C or U ribonucleotide, N′ indicates A, C G or U ribonucleotide in substrate, n indicates base complementary to the N′. The U4 and U7 positions in the catalytic core are indicated.

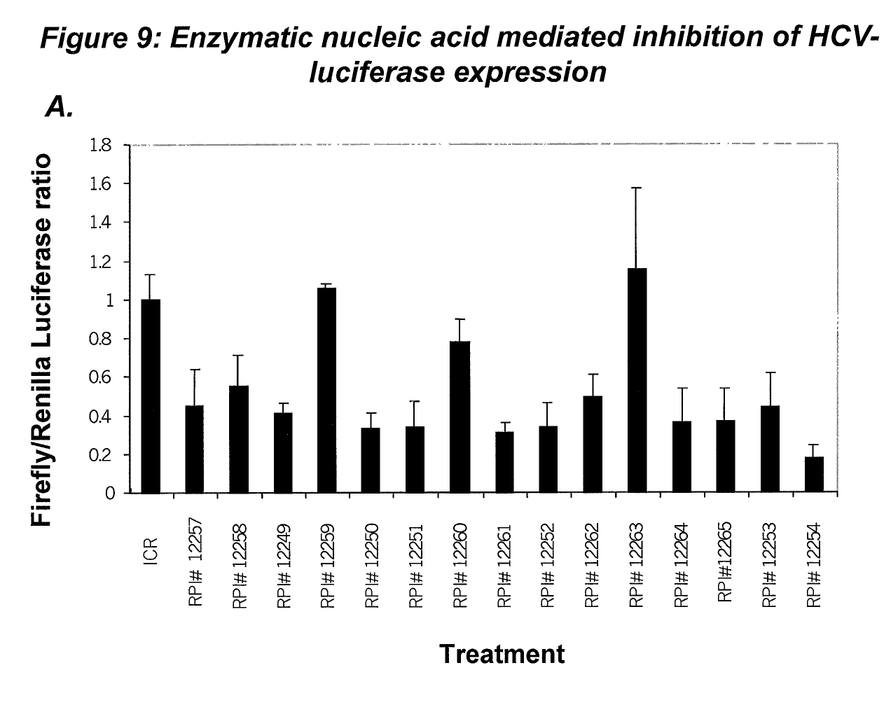

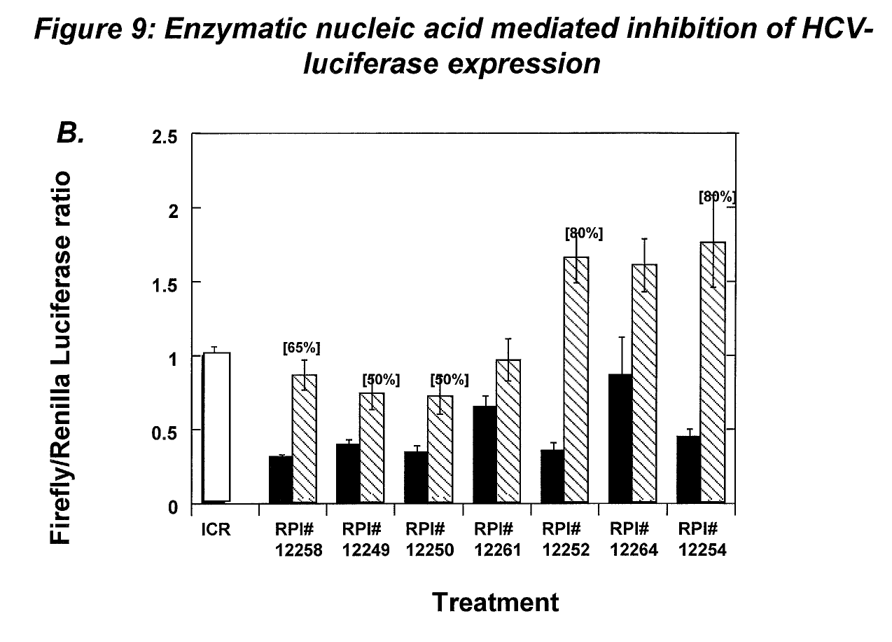

FIG. 9 is a set of bar graphs showing enzymatic nucleic acid mediated inhibition of HCV-luciferase expression in OST7 cells. OST7 cells were transfected with complexes containing reporter plasmids (2 μg/mL), enzymatic nucleic acids (100 nM) and lipid. The ratio of HCV-firefly luciferase luminescence/Renilla luciferase luminescence was determined for each enzymatic nucleic acid tested and was compared to treatment with the ICR, an irrelevant control enzymatic nucleic acid lacking specificity to the HCV 5′UTR (adjusted to 1). Results are reported as the mean of triplicate samples ±SD. In FIG. 9A, OST7 cells were treated with enzymatic nucleic acids (100 nM) targeting conserved sites (indicated by cleavage site) within the HCV 5′UTR. In FIG. 9B, OST7 cells were treated with a subset of enzymatic nucleic acids to lead HCV sites (indicated by cleavage site) and corresponding attenuated core (AC) controls. Percent decrease in firefly/Renilla luciferase ratio after treatment with active enzymatic nucleic acids as compared to treatment with corresponding ACs is shown when the decrease is ≧50% and statistically significant. Similar results were obtained with 50 nM enzymatic nucleic acid.

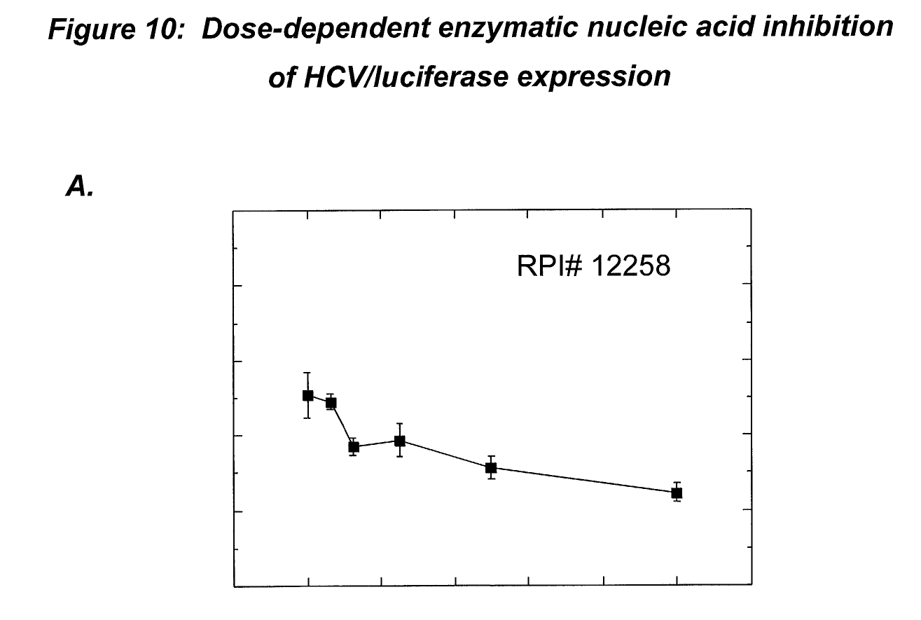

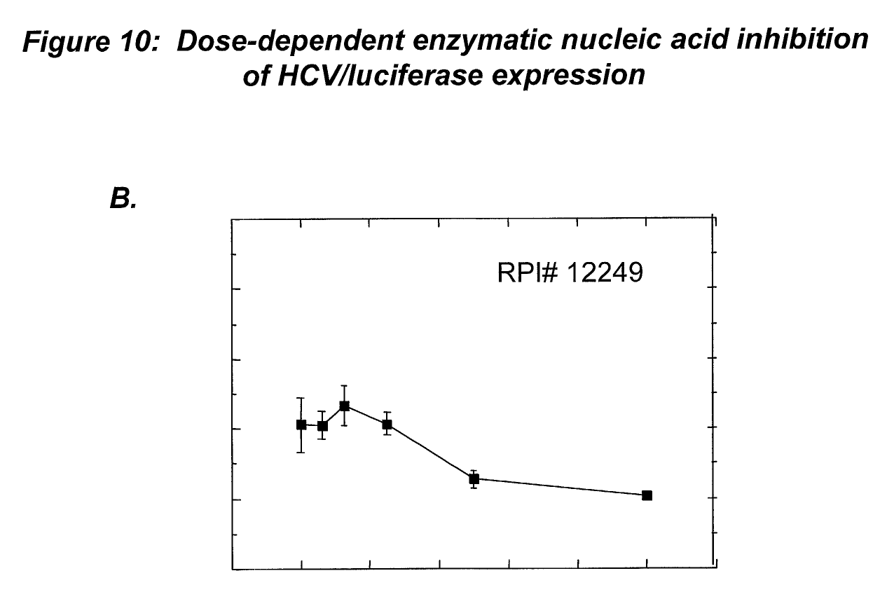

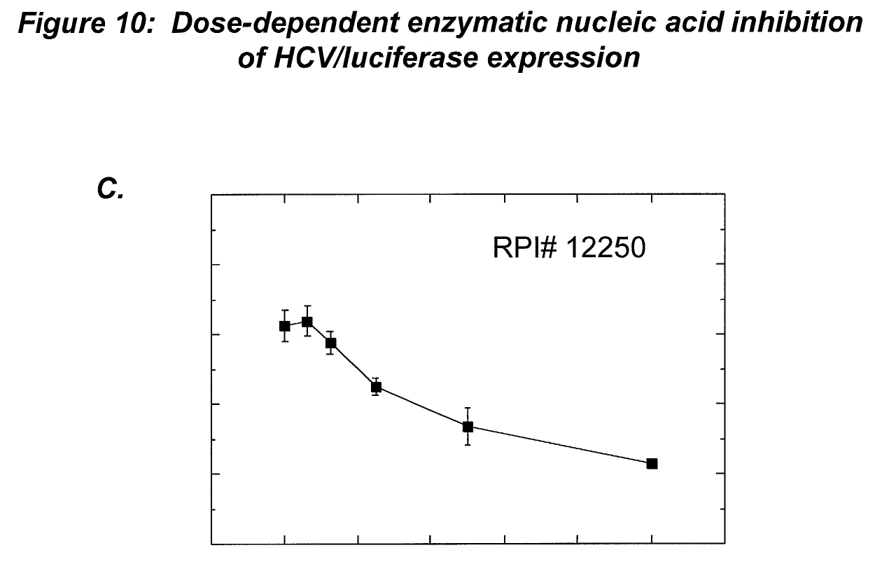

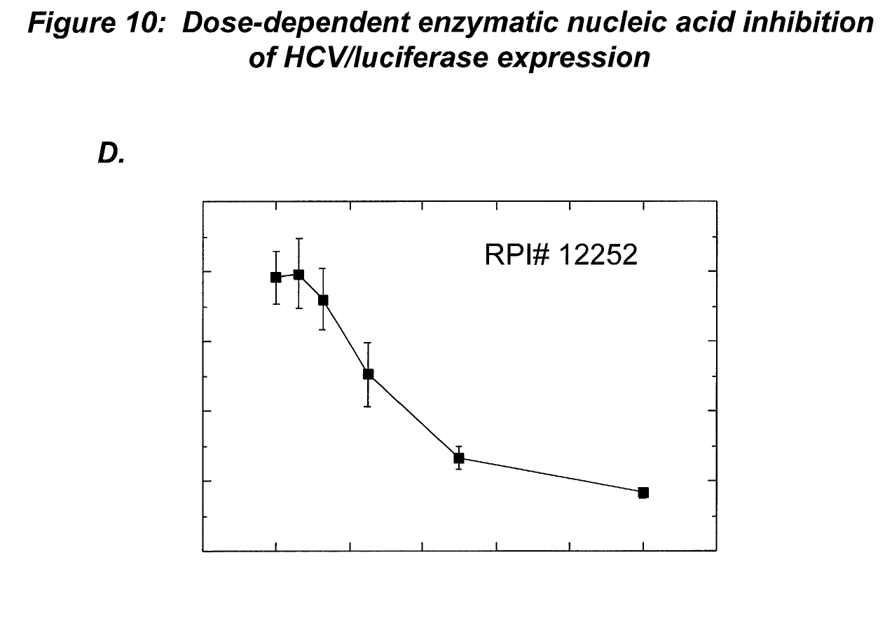

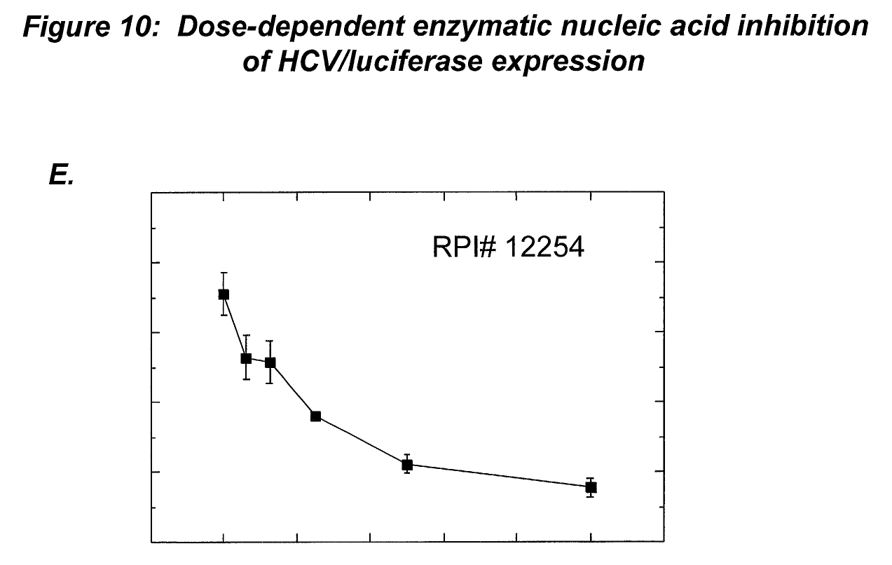

FIG. 10 is a series of line graphs showing the dose-dependent inhibition of HCV/luciferase expression following enzymatic nucleic acid treatment. Active enzymatic nucleic acid was mixed with corresponding AC to maintain a 100 nM total oligonucleotide concentration and the same lipid charge ratio. The concentration of active enzymatic nucleic acid for each point is shown. FIGS. 10A-E shows enzymatic nucleic acids targeting sites 79, 81, 142, 195, or 330, respectively. Results are reported as the mean of triplicate samples ±SD.

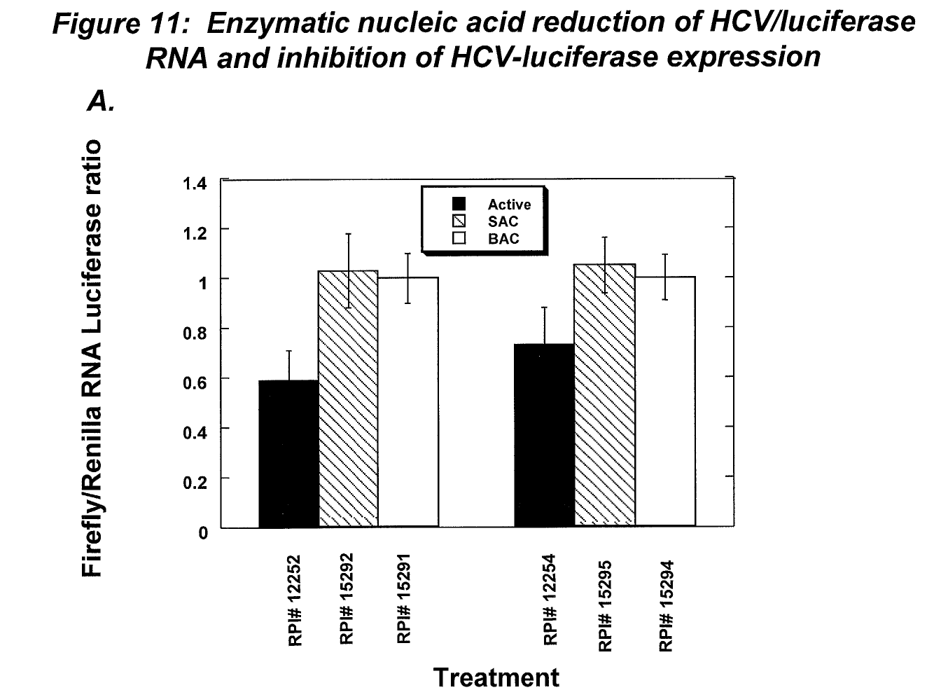

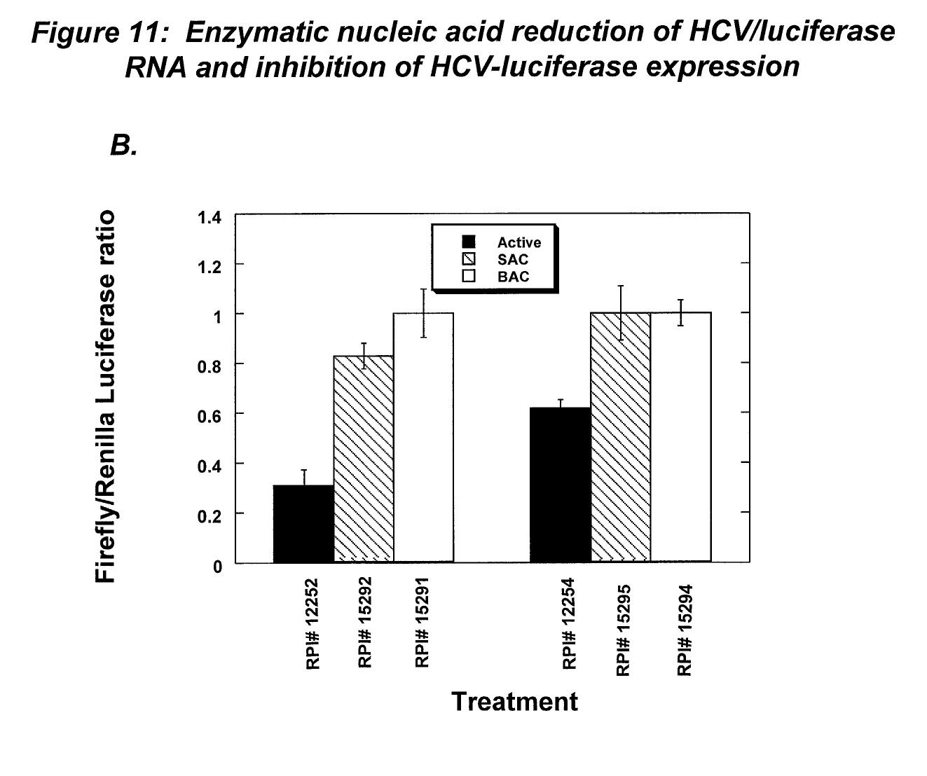

FIG. 11 is a set of bar graphs showing reduction of HCV/luciferase RNA and inhibition of HCV-luciferase expression in OST7 cells. OST7 cells were transfected with complexes containing reporter plasmids (2 μg/ml), enzymatic nucleic acids, BACs or SACs (50 nM) and lipid. Results are reported as the mean of triplicate samples ±SD. In FIG. 1A the ratio of HCV-firefly luciferase RNA/Renilla luciferase RNA is shown for each enzymatic nucleic acid or control tested. As compared to paired BAC controls (adjusted to 1), luciferase RNA levels were reduced by 40% and 25% for the site 195 or 330 enzymatic nucleic acids, respectively. In FIG. 11B the ratio of HCV-firefly luciferase luminescence/Renilla luciferase luminescence is shown after treatment with site 195 or 330 enzymatic nucleic acids or paired controls. As compared to paired BAC controls (adjusted to 1), inhibition of protein expression was 70% and 40% for the site 195 or 330 enzymatic nucleic acids, respectively P<0.01.

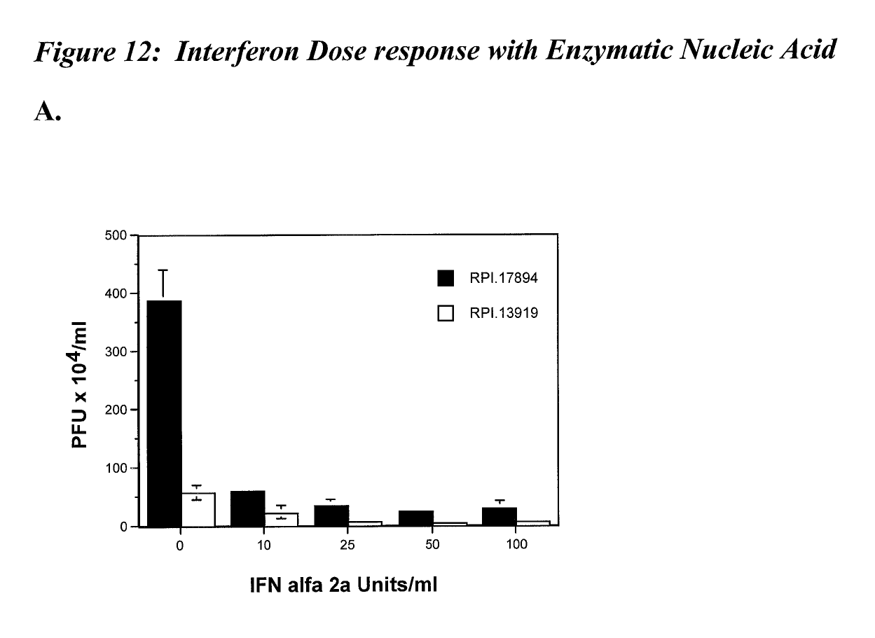

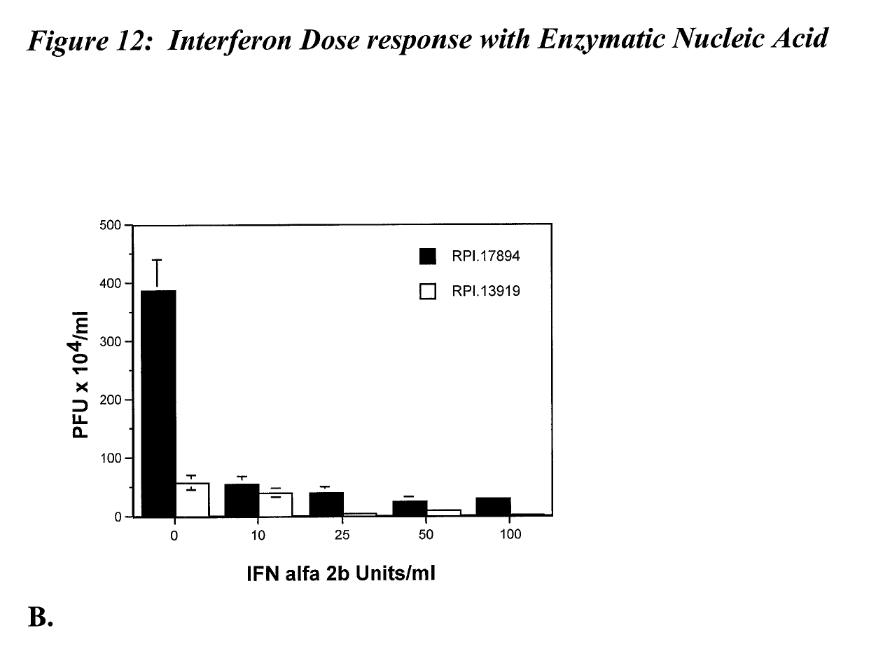

FIG. 12 is a set a bar graphs showing interferon (IFN) alpha 2a and 2b dose response in combination with site 195 anti-HCV enzymatic nucleic acid treatment. FIG. 12A shows data for IFN alfa 2a treatment. FIG. 12B shows data for IFN alfa 2b treatment. Viral yield is reported from HeLa cells pretreated with IFN in units/ml (U/ml) as indicated for 4 h prior to infection and then treated with either 200 nM control (SAC) or site 195 anti-HCV enzymatic nucleic acid (195 RZ) for 24 h after infection. Cells were infected with a MOI=0.1 for 30 min and collected at 24 h post infection. Error bars represent the S.D. of the mean of triplicate determinations.

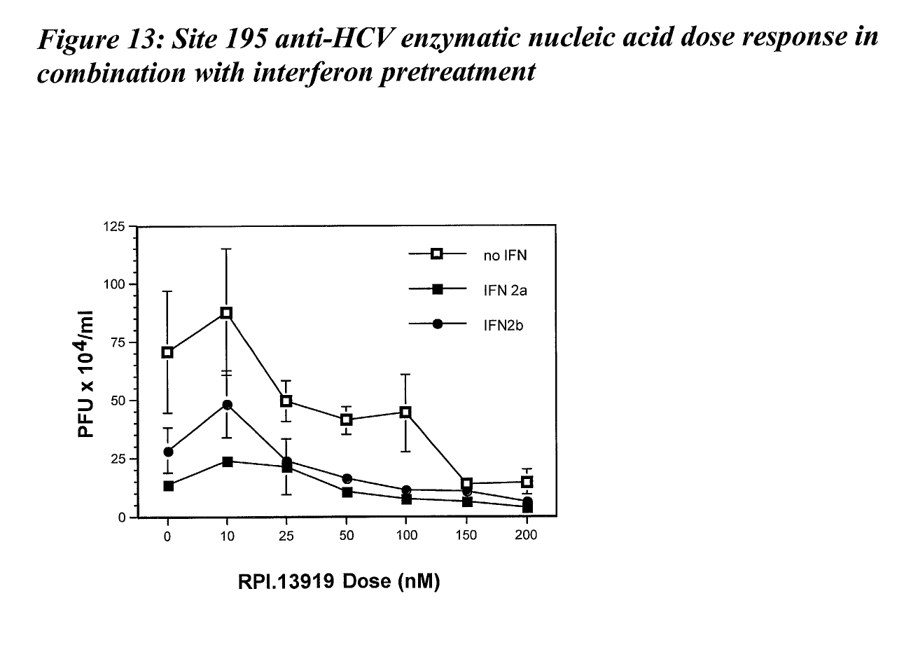

FIG. 13 is a line graph showing site 195 anti-HCV enzymatic nucleic acid dose response in combination with interferon (IFN) alpha 2a and 2b pretreatment. Viral yield is reported from HeLa cells pretreated for 4 h with or without IFN and treated with doses of site 195 anti-HCV enzymatic nucleic acid (195 RZ) as indicated for 24 h after infection. Anti-HCV enzymatic nucleic acid was mixed with control oligonucleotide (SAC) to maintain a constant 200 nM total dose of nucleic acid for delivery. Cells were infected with a MOI=0.1 for 30 min and collected at 24 h post infection. Error bars represent the S.D. of the mean of triplicate determinations.

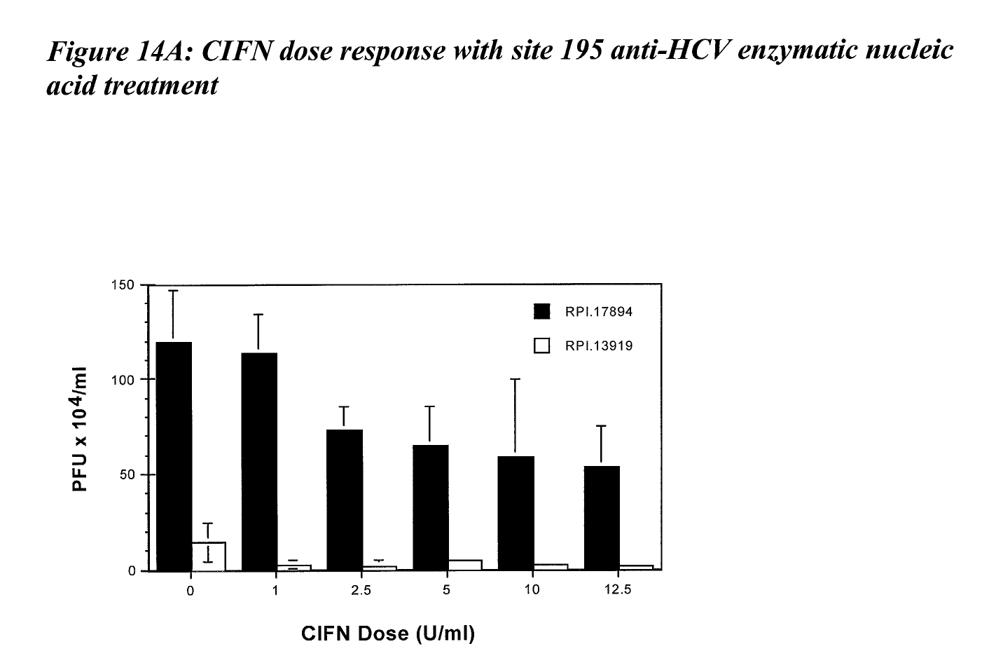

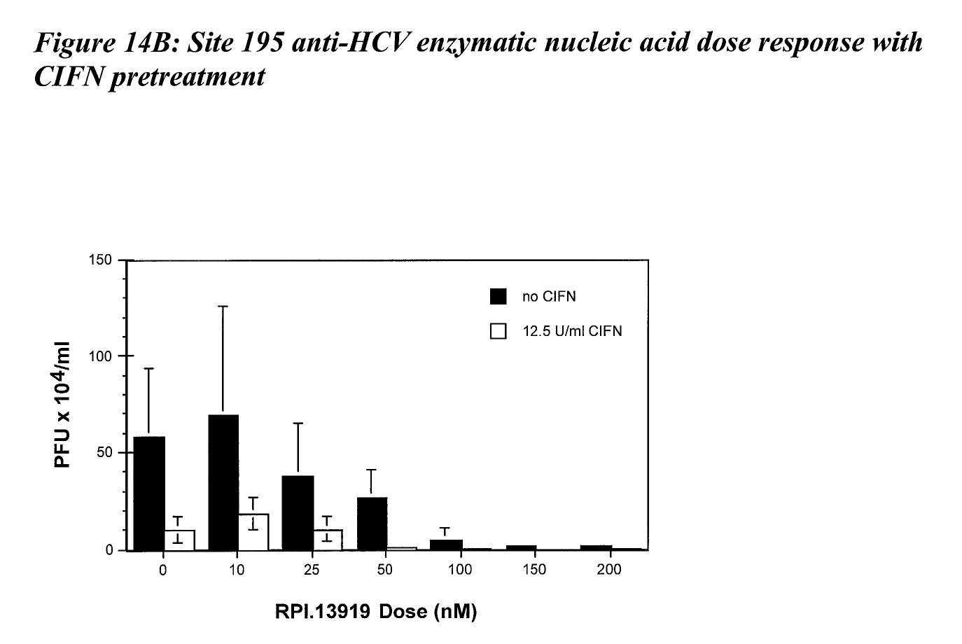

FIG. 14 is a set of bar graphs showing data from consensus interferon (CIFN)/enzymatic nucleic acid combination treatment. FIG. 14A shows CIFN dose response with site 195 anti-HCV enzymatic nucleic acid treatment. Viral yield is reported from cells pretreated with CIFN in units/ml (U/ml) as indicated and treated with either 200 nM control (SAC) or site 195 anti-HCV enzymatic nucleic acid (195 RZ). FIG. 14B shows site 195 anti-HCV enzymatic nucleic acid dose response with CIFN pretreatment. Viral yield is reported from cells pretreated with or without CIFN and treated with concentrations of site 195 anti-HCV enzymatic nucleic acid (195 RZ) as indicated. Anti-HCV enzymatic nucleic acid was mixed with control oligonucleotide (SAC) to maintain a constant 200 nM total dose of nucleic acid for delivery. Cells were infected with a MOI=0.1 for 30 min. and collected at 24 h post infection. Error bars represent the S.D. of the mean of triplicate determinations.

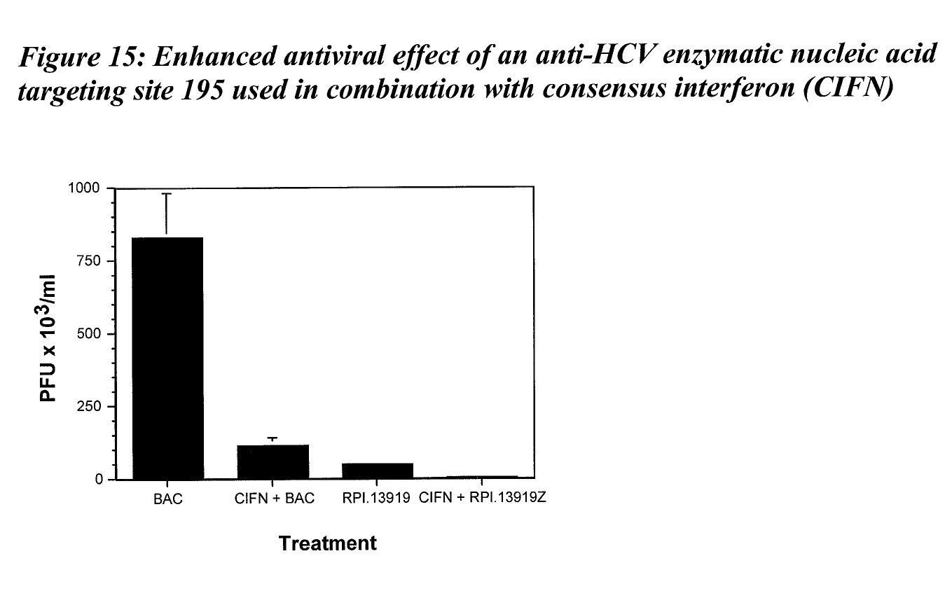

FIG. 15 is a bar graph showing enzymatic nucleic acid activity and enhanced antiviral effect of an anti-HCV enzymatic nucleic acid targeting site 195 used in combination with consensus interferon (CIFN). Viral yield is reported from cells treated as indicated. BAC, cells were treated with 200 nM BAC (binding attenuated control) for 24 h after infection; CIFN+BAC, cells were treated with 12.5 U/ml CIFN for 4 h prior to infection and with 200 nM BAC for 24 h after infection; 195 RZ, cells were treated with 200 nM site 195 anti-HCV enzymatic nucleic acid for 24 h after infection; CIFN+195 RZ, cells were treated with 12.5 U/ml CIFN for 4 h prior to infection and with 200 nM site 195 anti-HCV enzymatic nucleic acid for 24 h after infection. Cells were infected with a MOI=0.1 for 30 min. Error bars represent the S.D. of the mean of triplicate determinations.

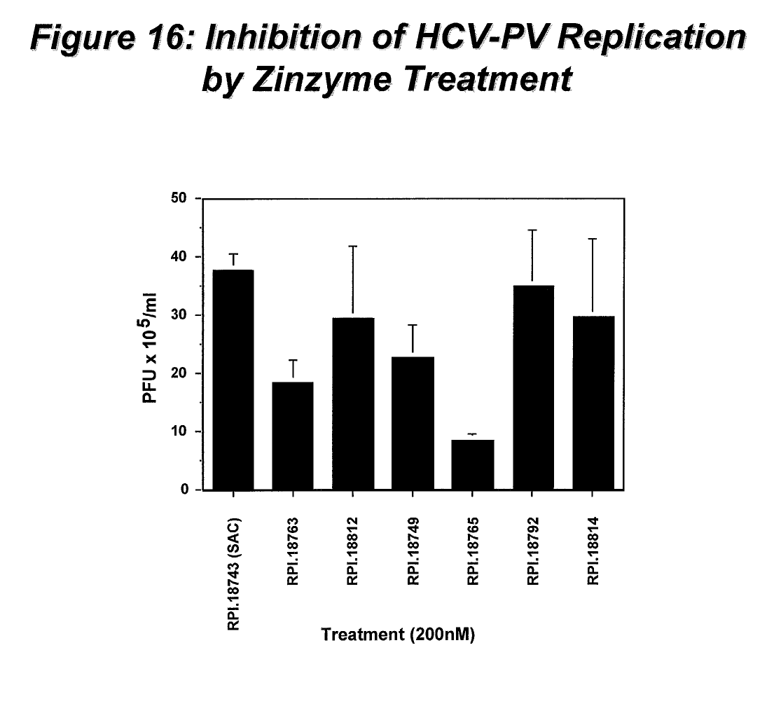

FIG. 16 is a bar graph showing inhibition of a HCV-PV chimera replication by treatment with zinzyme enzymatic nucleic acid molecules targeting different sites within the HCV 5′-UTR compared to a scrambled attenuated core control (SAC) zinzyme.

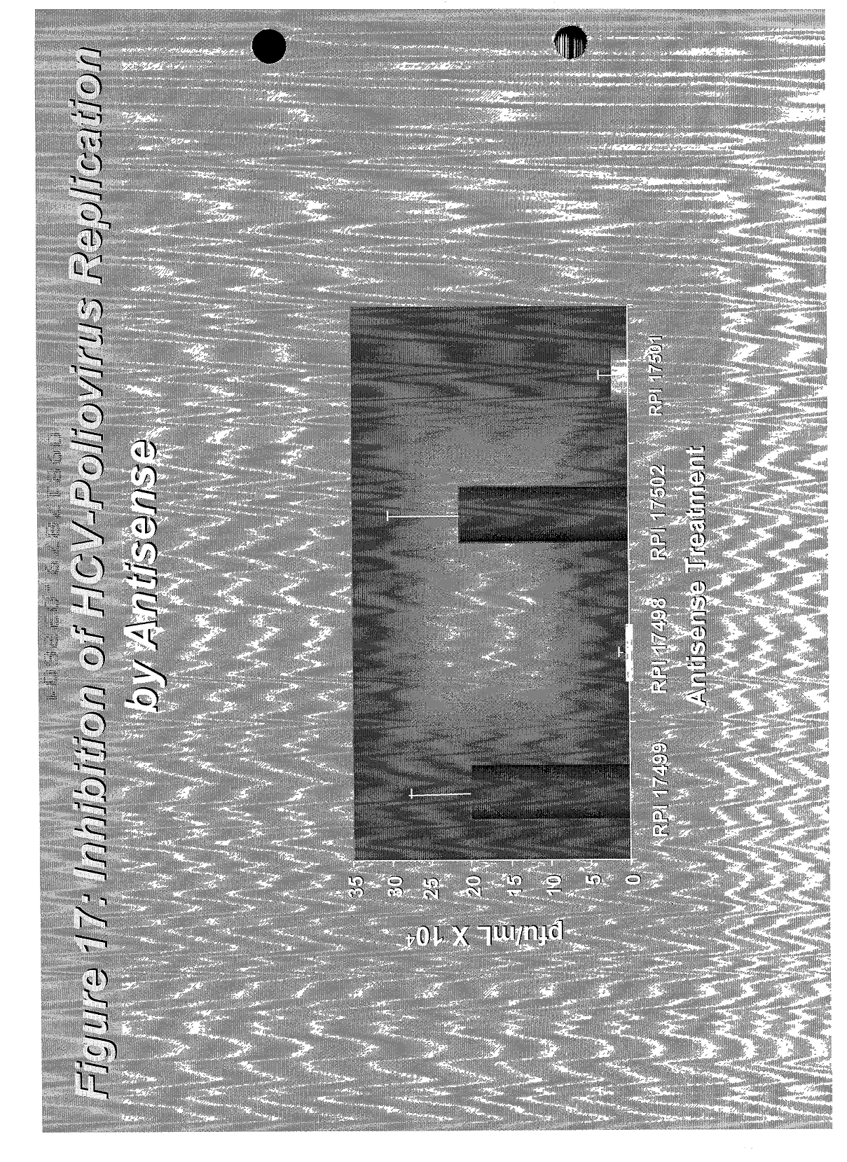

FIG. 17 is a bar graph showing inhibition of a HCV-PV chimera replication by antisense nucleic acid molecules targeting conserved regions of the HCV 5′-UTR compared to scrambled antisense controls.

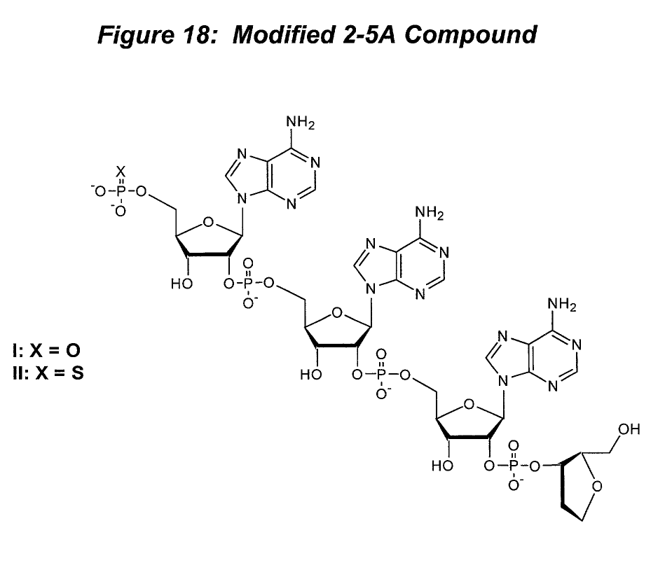

FIG. 18 shows the structure of compounds (2-5A) utilized in the study. “X” denotes the position of oxygen (0) in analog I or sulfur (S) in thiophosphate (P═S) analog II. The 2-5A compounds were synthesized, deprotected and purified as described herein utilizing CPG support with 3′-inverted abasic nucleotide. For chain extension 5′-O-(4,4′-dimetoxytrityl)-3′-O-(tert-butyldimethylsilyl)-N6-benzoyladenosine-2-cyanoethyl-N,N-diisopropyl-phosphoramidite (Chem. Genes Corp., Waltham, Mass.) was employed. Introduction of a 5′-terminal phosphate (analog I) or thiophosphate (analog II) group was performed with “Chemical Phosphorylation Reagent” (Glen Research, Sterling, Va.). Structures of the final compounds were confirmed by MALDI-TOF analysis.

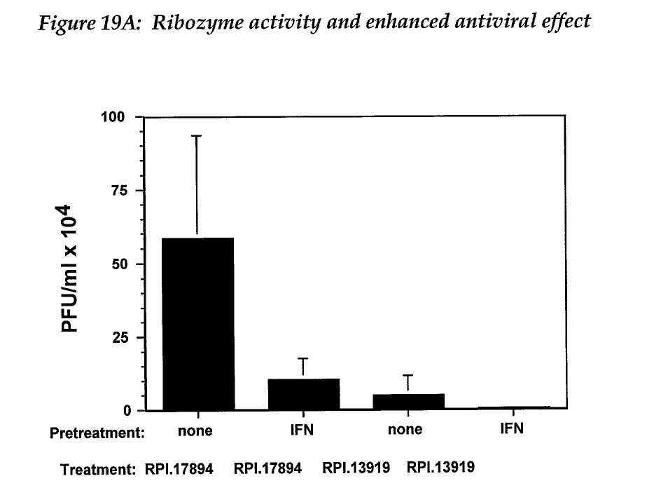

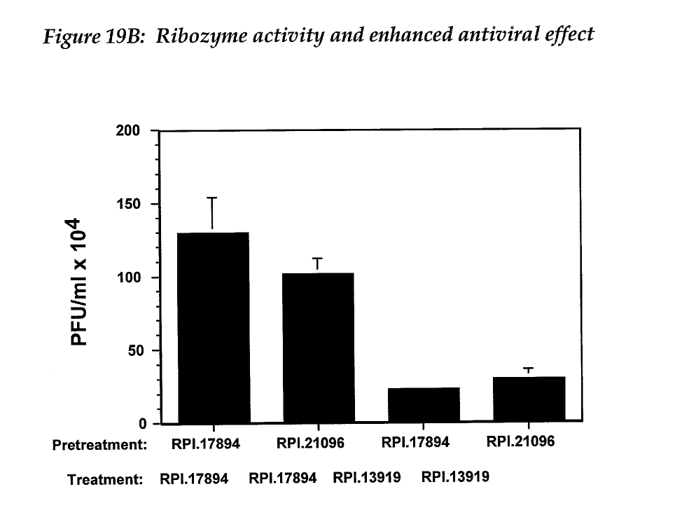

FIG. 19 is a bar graph showing ribozyme activity and enhanced antiviral effect. (A) Interferon/ribozyme combination treatment. (B) 2-5A/ribozyme combination treatment. HeLa cells seeded in 96-well plates (10,000 cells per well) were pretreated as indicated for 4 hours. For pretreatment, SAC (RPI 17894), RZ (RPI 13919), and 2-5A analog I (RPI 21096) (200 nM) were complexed with lipid cytofectin. Cells were then infected with HCV-PV at a multiplicity of infection of 0.1. Virus inoculum was replaced after 30 minutes with media containing 5% serum and 100 nM RZ or SAC as indicated, complexed with cytofectin RPI.9778. After 20 hours, cells were lysed by 3 freeze/thaw cycles and virus was quantified by plaque assay. Plaque forming units (PFU)/ml are shown as the mean of triplicate samples +SEM. The absolute amount of viral yield in treated cells varied from day to day, presumably due to day to day variations in cell plating and transfection complexation. None, normal media; IFN, 10 U/ml consensus interferon; SAC, scrambled arm attenuated core control (RPI 17894); RZ, anti-HCV ribozyme (RPI 13919); 2-5A, (RPI 21096).

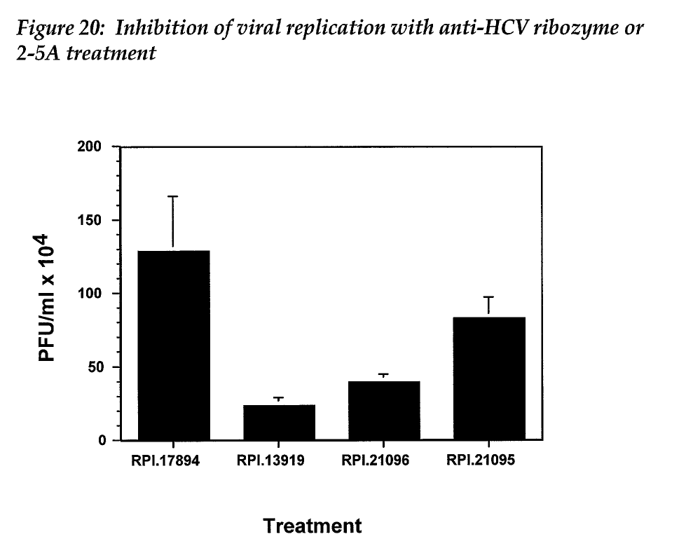

FIG. 20 is a graph showing the inhibition of viral replication with anti-HCV ribozyme (RPI 13919) or 2-5A (RPI 21096) treatment. HeLa cells were treated as described in FIG. 19 except that there was no pretreatment and 200 nM oligonucleotide was used for treatment. 2-5A P═S contains a 5′-terminal thiophosphate (RPI21095) (see FIG. 18).

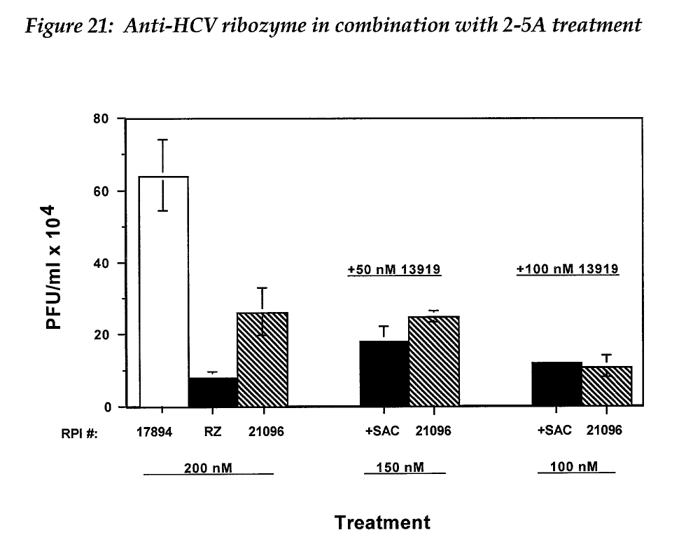

FIG. 21 is a bar graph showing anti-HCV ribozyme in combination with 2-5A treatment. HeLa cells were treated as described in FIG. 20 except concentrations were co-varied as shown to maintain a constant 200 nM total oligonucleotide dose for transfection. Cells treated with 50 nM anti-HCV ribozyme (RPI 13919) (middle bars) were also treated with 150 nM SAC (RPI 17894) or 2-5A (RPI 21096); likewise, cells treated with 100 nM anti-HCV ribozyme (bars at right) were also treated with 100 nM SAC or 2-5A.

### MECHANISM OF ACTION OF NUCLEIC ACID MOLECULES OF THE INVENTION

Antisense:

Antisense molecules can be modified or unmodified RNA, DNA, or mixed polymer oligonucleotides and primarily function by specifically binding to matching sequences resulting in inhibition of peptide synthesis (Wu-Pong, November 1994, BioPharm, 20-33). The antisense oligonucleotide binds to target RNA by Watson Crick base-pairing and blocks gene expression by preventing ribosomal translation of the bound sequences either by steric blocking or by activating RNase H enzyme. Antisense molecules can also alter protein synthesis by interfering with RNA processing or transport from the nucleus into the cytoplasm (Mukhopadhyay & Roth, 1996, Crit. Rev. in Oncogenesis 7, 151-190).

In addition, binding of single stranded DNA to RNA can result in nuclease degradation of the heteroduplex (Wu-Pong, supra; Crooke, supra). To date, the only backbone modified DNA chemistry which will act as substrates for RNase H are phosphorothioates, phosphorodithioates, and borontrifluoridates. Recently it has been reported that 2′-arabino and 2′-fluoro arabino-containing oligos can also activate RNase H activity.

A number of antisense molecules have been described that utilize novel configurations of chemically modified nucleotides, secondary structure, and/or RNase H substrate domains (Woolf et al., International PCT Publication No. WO 98/13526; Thompson et al., International PCT Publication No. WO 99/54459; Hartmann et al., U.S. Ser. No. 60/101,174 which was filed on Sep. 21, 1998) all of these are incorporated by reference herein in their entirety.

In addition, antisense deoxyoligoribonucleotides can be used to target RNA by means of DNA-RNA interactions, thereby activating RNase H, which digests the target RNA in the duplex. Antisense DNA can be expressed via the use of a single stranded DNA intracellular expression vector or equivalents and variations thereof.

2-5A Antisense Chimera:

The 2-5A system is an interferon mediated mechanism for RNA degradation found in higher vertebrates (Mitra et al., 1996, Proc Nat Acad Sci USA 93, 6780-6785). Two types of enzymes, 2-5A synthetase and RNase L, are required for RNA cleavage. The 2-5A synthetases require double stranded RNA to form 2′-5′ oligoadenylates (2-5A). 2-5A then acts as an allosteric effector for utilizing RNase L which has the ability to cleave single stranded RNA. The ability to form 2-5A structures with double stranded RNA makes this system particularly useful for inhibition of viral replication.

(2′-5′) oligoadenylate structures can be covalently linked to antisense molecules to form chimeric oligonucleotides capable of RNA cleavage (Torrence, supra). Alternatively, (2′-5′) oligoadenylate structures can be covalently linked to enzymatic nucleic acid molecules to form chimeric oligonucleotides capable of RNA cleavage. These molecules putatively bind and activate a 2-5A dependent RNase, the oligonucleotide/enzyme complex then binds to a target RNA molecule which can then be cleaved by the RNase enzyme and the enzymatic nucleic acid.

Enzymatic Nucleic Acid Molecules

There are several known classes of enzymatic nucleic acid molecules capable of cleaving target RNA. In addition, several in vitro selection (evolution) strategies (Orgel, 1979, Proc. R. Soc. London, B 205, 435) have been used to evolve new nucleic acid catalysts capable of catalyzing cleavage and ligation of phosphodiester linkages (Joyce, 1989, Gene, 82, 83-87; Beaudry et al., 1992, Science 257, 635-641; Joyce, 1992, Scientific American 267, 90-97; Breaker et al., 1994, TIBTECH 12, 268; Bartel et al., 1993, Science 261:1411-1418; Szostak, 1993, TIBS 17, 89-93; Kumar et al., 1995, FASEB J., 9, 1183; Breaker, 1996, Curr. Op. Biotech., 7, 442; Santoro et al., 1997, Proc. Natl. Acad. Sci., 94, 4262; Tang et al., 1997, RNA 3, 914; Nakamaye & Eckstein, 1994, supra; Long & Uhlenbeck, 1994, supra; Ishizaka et al., 1995, supra; Vaish et al., 1997, Biochemistry 36, 6495; all of these are incorporated by reference herein). Each can catalyze a series of reactions including the hydrolysis of phosphodiester bonds in trans (and thus can cleave other RNA molecules) under physiological conditions. Table I summarizes some of the characteristics of some of these enzymatic nucleic acids. In general, enzymatic nucleic acids act by first binding to a target RNA. Such binding occurs through the target binding portion of an enzymatic nucleic acid which is held in close proximity to an enzymatic portion of the molecule that acts to cleave the target RNA. Thus, the enzymatic nucleic acid first recognizes and then binds a target RNA through complementary base-pairing, and once bound to the correct site, acts enzymatically to cut the target RNA. Strategic cleavage of such a target RNA destroys its ability to direct synthesis of an encoded protein. After an enzymatic nucleic acid has bound and cleaved its RNA target, it is released from that RNA to search for another target and can repeatedly bind and cleave new targets.

The enzymatic nature of an enzymatic nucleic acid molecule is advantageous over other technologies, since the concentration of enzymatic nucleic acid molecule necessary to affect a therapeutic treatment is lower. This advantage reflects the ability of the enzymatic nucleic acid molecule to act enzymatically. Thus, a single enzymatic nucleic acid molecule is able to cleave many molecules of target RNA. In addition, the enzymatic nucleic acid molecule is a highly specific inhibitor, with the specificity of inhibition depending not only on the base-pairing mechanism of binding to the target RNA, but also on the mechanism of target RNA cleavage. Single mismatches, or base-substitutions, near the site of cleavage can be chosen to completely eliminate catalytic activity of an enzymatic nucleic acid molecule.

Nucleic acid molecules having an endonuclease enzymatic activity are able to repeatedly cleave other separate RNA molecules in a nucleotide base sequence-specific manner. Such enzymatic nucleic acid molecules can be targeted to virtually any RNA transcript, and efficient cleavage achieved in vitro (Zaug et al., 324, Nature 429 1986; Uhlenbeck, 1987 Nature 328, 596; Kim et al., 84 Proc. Natl. Acad. Sci. USA 8788, 1987; Dreyfus, 1988, Einstein Quart. J. Bio. Med., 6, 92; Haseloff and Gerlach, 334 Nature 585, 1988; Cech, 260 JAMA 3030, 1988; and Jefferies et al., 17 Nucleic Acids Research 1371, 1989; Chartrand et al., 1995, Nucleic Acids Research 23, 4092; Santoro et al., 1997, PNAS 94, 4262).

Because of their sequence-specificity, trans-cleaving enzymatic nucleic acid molecules show promise as therapeutic agents for human disease (Usman & McSwiggen, 1995 Ann. Rep. Med. Chem. 30, 285-294; Christoffersen and Marr, 1995 J. Med. Chem. 38, 2023-2037). Enzymatic nucleic acid molecules can be designed to cleave specific RNA targets within the background of cellular RNA. Such a cleavage event renders the RNA non-functional and abrogates protein expression from that RNA. In this manner, synthesis of a protein associated with a disease state can be selectively inhibited.

Enzymatic nucleic acid molecules that cleave the specified sites in HCV RNAs represent a novel therapeutic approach to infection by the hepatitis C virus. As shown herein, enzymatic nucleic acids are able to inhibit the activity of HCV and the catalytic activity of the enzymatic nucleic acids is required for their inhibitory effect. Those of ordinary skill in the art will find that it is clear from the examples described that other enzymatic nucleic acid molecules that cleave HCV RNAs can be readily designed and are within the invention.

Target sites

Targets for useful nucleic acid molecules and nuclease activating compounds or chimeras can be determined as disclosed in Draper et al., WO 93/23569; Sullivan et al., WO 93/23057; Thompson et al., WO 94/02595; Draper et al., WO 95/04818; McSwiggen et al., U.S. Pat. No. 5,525,468 and hereby incorporated by reference herein in totality. Rather than repeat the guidance provided in those documents here, below are provided specific examples of such methods, not limiting to other available methods known in the art. Nucleic acid molecules and nuclease activating compounds or chimeras to such targets are designed as described in those applications and synthesized to be tested in vitro and in vivo, as also described. Such nucleic acid molecules and nuclease activating compounds or chimeras can also be optimized and delivered as described therein.

The sequence of HCV RNAs were screened for optimal enzymatic nucleic acid molecule target sites using a computer folding algorithm. Enzymatic nucleic acid cleavage sites were identified. These sites are shown in Tables III, IV, V and VIII (All sequences are 5′ to 3′ in the tables). The nucleotide base position is noted in the tables as that site to be cleaved by the designated type of enzymatic nucleic acid molecule. The nucleotide base position is noted in the tables as that site to be cleaved by the designated type of enzymatic nucleic acid molecule.

Because HCV RNAs are highly homologous in certain regions, some enzymatic nucleic acid molecule target sites are also homologous. In this case, a single enzymatic nucleic acid molecule will target different classes of HCV RNA. The advantage of one enzymatic nucleic acid molecule that targets several classes of HCV RNA is clear, especially in cases where one or more of these RNAs can contribute to the disease state.

Enzymatic nucleic acid molecules were designed that could bind and were individually analyzed by computer folding (Jaeger et al., 1989 Proc. Natl. Acad. Sci. USA, 86, 7706) to assess whether the enzymatic nucleic acid molecule sequences fold into the appropriate secondary structure. Those enzymatic nucleic acid molecules with unfavorable intramolecular interactions between the binding arms and the catalytic core are eliminated from consideration. Varying binding arm lengths can be chosen to optimize activity. Generally, at least 4 bases on each arm are able to bind to, or otherwise interact with, the target RNA. Enzymatic nucleic acid molecules were designed to anneal to various sites in the mRNA message. The binding arms are complementary to the target site sequences described above.

Nucleic Acid Synthesis

Synthesis of nucleic acids greater than 100 nucleotides in length is difficult using automated methods, and the therapeutic cost of such molecules is prohibitive. In this invention, small nucleic acid motifs (“small refers to nucleic acid motifs no more than 100 nucleotides in length, preferably no more than 80 nucleotides in length, and most preferably no more than 50 nucleotides in length; e.g., antisense oligonucleotides, hammerhead or the Inozyme enzymatic nucleic acids) are preferably used for exogenous delivery. The simple structure of these molecules increases the ability of the nucleic acid to invade targeted regions of RNA structure. Exemplary molecules of the instant invention are chemically synthesized, and others can similarly be synthesized.

The method of synthesis used for normal RNA including certain enzymatic nucleic acid molecules follows the procedure as described in Usman et al., 1987, J. Am. Chem. Soc., 109, 7845; Scaringe et al., 1990, Nucleic Acids Res., 18, 5433; and Wincott et al., 1995, Nucleic Acids Res. 23, 2677-2684 Wincott et al., 1997, Methods Mol. Bio., 74, 59, and makes use of common nucleic acid protecting and coupling groups, such as dimethoxytrityl at the 5′-end, and phosphoramidites at the 3′-end. In a non-limiting example, small scale syntheses are conducted on a 394 Applied Biosystems, Inc. synthesizer using a 0.2 μmol scale protocol with a 7.5 min coupling step for alkylsilyl protected nucleotides and a 2.5 min coupling step for 2′-O-methylated nucleotides. Table II outlines the amounts and the contact times of the reagents used in the synthesis cycle. Alternatively, syntheses at the 0.2 μmol scale can be done on a 96-well plate synthesizer, such as the instrument produced by Protogene (Palo Alto, Calif.) with minimal modification to the cycle. A 33-fold excess (60 μL of 0.11 M=6.6 mol) of 2′-O-methyl phosphoramidite and a 75-fold excess of S-ethyl tetrazole (60 μL of 0.25 M=15 μmol) can be used in each coupling cycle of 2′-O-methyl residues relative to polymer-bound 5′-hydroxyl. A 66-fold excess (120 μL of 0.11 M=13.2 μmol) of alkylsilyl (ribo) protected phosphoramidite and a 150-fold excess of S-ethyl tetrazole (120 μL of 0.25 M=30 μmol) can be used in each coupling cycle of ribo residues relative to polymer-bound 5′-hydroxyl. Average coupling yields on the 394 Applied Biosystems, Inc. synthesizer, determined by colorimetric quantitation of the trityl fractions, are typically 97.5-99%. Other oligonucleotide synthesis reagents for the 394 Applied Biosystems, Inc. synthesizer include; detritylation solution is 3% TCA in methylene chloride (ABI); capping is performed with 16% N-methyl imidazole in THF (ABI) and 10% acetic anhydride/10% 2,6-lutidine in THF (ABI); oxidation solution is 16.9 mM I₂, 49 mM pyridine, 9% water in THF (PERSEPTIVE™). Burdick & Jackson Synthesis Grade acetonitrile is used directly from the reagent bottle. S-Ethyltetrazole solution (0.25 M in acetonitrile) is made up from the solid obtained from American International Chemical, Inc. Alternately, for the introduction of phosphorothioate linkages, Beaucage reagent (3H-1,2-Benzodithiol-3-one 1,1-dioxide 0.05 M in acetonitrile) is used.

Deprotection of the RNA is performed using either a two-pot or one-pot protocol. For the two-pot protocol, the polymer-bound trityl-on oligoribonucleotide is transferred to a 4 mL glass screw top vial and suspended in a solution of 40% aq. methylamine (1 mL) at 65° C. for 10 min. After cooling to −20° C., the supernatant is removed from the polymer support. The support is washed three times with 1.0 mL of EtOH:MeCN:H2O/3:1:1, vortexed and the supernatant is then added to the first supernatant. The combined supernatants, containing the oligoribonucleotide, are dried to a white powder. The base deprotected oligoribonucleotide is resuspended in anhydrous TEA/HF/NMP solution (300 μL of a solution of 1.5 mL N-methylpyrrolidinone, 750 μL TEA and 1 mL TEA-3HF to provide a 1.4 M HF concentration) and heated to 65° C. After 1.5 h, the oligomer is quenched with 1.5 M NH₄HCO₃.

Alternatively, for the one-pot protocol, the polymer-bound trityl-on oligoribonucleotide is transferred to a 4 mL glass screw top vial and suspended in a solution of 33% ethanolic methylamine/DMSO: 1/1 (0.8 mL) at 65° C. for 15 min. The vial is brought to r.t. TEA.3HF (0.1 mL) is added and the vial is heated at 65° C. for 15 min. The sample is cooled at −20° C. and then quenched with 1.5 M NH₄HCO₃.

For anion exchange desalting of the deprotected oligomer, the TEAB solution was loaded onto a Qiagen 500® anion exchange cartridge (Qiagen Inc.) that was prewashed with 50 mM TEAB (10 mL). After washing the loaded cartridge with 50 mM TEAB (10 mL), the RNA was eluted with 2 M TEAB (10 mL) and dried down to a white powder.

For purification of the trityl-on oligomers, the quenched NH₄HCO₃ solution is loaded onto a C-18 containing cartridge that had been prewashed with acetonitrile followed by 50 mM TEAA. After washing the loaded cartridge with water, the RNA is detritylated with 0.5% TFA for 13 min. The cartridge is then washed again with water, salt exchanged with 1 M NaCl and washed with water again. The oligonucleotide is then eluted with 30% acetonitrile.

Inactive hammerhead enzymatic nucleic acids were synthesized by substituting switching the order of G₅A₆ and substituting a U for A₁₄(numbering from Hertel, K. J., et al., 1992, Nucleic Acids Res., 20, 3252). Inactive enzymatic nucleic acids can also be synthesized by substituting a U for G5 and a U for A14. In some cases, the sequence of the substrate binding arms were randomized while the overall base composition was maintained.

The average stepwise coupling yields are typically >98% (Wincott et al, 1995 Nucleic Acids Res. 23, 2677-2684). Those of ordinary skill in the art will recognize that the scale of synthesis can be adapted to be larger or smaller than the example described above including but not limited to 96 well format, all that is important is the ratio of chemicals used in the reaction.

Enzymatic nucleic acid molecules can be synthesized in two parts and annealed to reconstruct the active enzymatic nucleic acid molecule (Chowrira and Burke, 1992 Nucleic Acids Res., 20, 2835-2840). Enzymatic nucleic acid molecules can also be synthesized from DNA templates using bacteriophage T7 RNA polymerase (Milligan and Uhlenbeck, 1989, Methods Enzymol. 180, 51). DNAzymes can be synthesized chemically or expressed endogenously in vivo, by means of a single stranded DNA vector or equivalent thereof.

Alternatively, the nucleic acid molecules of the present invention can be synthesized separately and joined together post-synthetically, for example by ligation (Moore et al., 1992, Science 256, 9923; Draper et al., International PCT publication No. WO 93/23569; Shabarova et al., 1991, Nucleic Acids Research 19, 4247; Bellon et al., 1997, Nucleosides & Nucleotides, 16, 951; Bellon et al., 1997, Bioconjugate Chem. 8, 204).

The nucleic acid molecules of the present invention are modified extensively to enhance stability by modification with nuclease resistant groups, for example, 2′-amino, 2′-C-allyl, 2′-flouro, 2′-O-methyl, 2′-H (for a review see Usman and Cedergren, 1992, TIBS 17, 34; Usman et al., 1994, Nucleic Acids Symp. Ser. 31, 163). Enzymatic nucleic acids are purified by gel electrophoresis using general methods or are purified by high pressure liquid chromatography (HPLC; See Wincott et al., Supra, the totality of which is hereby incorporated herein by reference) and are re-suspended in water.

The sequences of the nucleic acid molecules that are chemically synthesized, useful in this study, are shown in Tables V-VIII. Those in the art will recognize that these sequences are representative only of many more such sequences where the enzymatic portion of the enzymatic nucleic acid (all but the binding arms) is altered to affect activity. The nucleic acid sequences listed in Tables V-VIII can be formed of ribonucleotides or other nucleotides or non-nucleotides. Such nucleic acid molecules with enzymatic activity are equivalent to the enzymatic nucleic acid molecules described specifically in the tables.

Optimizing Activity of the Nucleic Acid Molecules of the Invention.

Chemically synthesizing nucleic acid molecules with modifications (base, sugar and/or phosphate) that prevent their degradation by serum ribonucleases can increase their potency (see e.g., Eckstein et al., International Publication No. WO 92/07065; Perrault et al., 1990 Nature 344, 565; Pieken et al., 1991, Science 253, 314; Usman and Cedergren, 1992, Trends in Biochem. Sci. 17, 334; Usman et al., International Publication No. WO 93/15187; and Rossi et al., International Publication No. WO 91/03162; Sproat, U.S. Pat. No. 5,334,711; and Burgin et al., supra; all of these describe various chemical modifications that can be made to the base, phosphate and/or sugar moieties of the nucleic acid molecules herein). Modifications which enhance their efficacy in cells, and removal of bases from nucleic acid molecules to shorten oligonucleotide synthesis times and reduce chemical requirements are desired. (All these publications are hereby incorporated by reference herein).

There are several examples in the art describing sugar, base and phosphate modifications that can be introduced into nucleic acid molecules with significant enhancement in their nuclease stability and efficacy. For example, oligonucleotides are modified to enhance stability and/or enhance biological activity by modification with nuclease resistant groups, for example, 2′-amino, 2′-C-allyl, 2′-flouro, 2′-O-methyl, 2′-H, nucleotide base modifications (for a review see Usman and Cedergren, 1992, TIBS. 17, 34; Usman et al. 1994, Nucleic Acids Symp. Ser. 31, 163; Burgin et al., 1996, Biochemistry, 35, 14090). Sugar modification of nucleic acid molecules have been extensively described in the art (see Eckstein et al., International Publication PCT No. WO 92/07065; Perrault et al. Nature, 1990, 344, 565-568; Pieken et al. Science, 1991, 253, 314-317; Usman and Cedergren, Trends in Biochem. Sci., 1992, 17, 334-339; Usman et al. International Publication PCT No. WO 93/15187; Sproat, U.S. Pat. No. 5,334,711 and Beigelman et al., 1995, J. Biol. Chem., 270, 25702; Beigelman et al., International PCT publication No. WO 97/26270; Beigelman et al., U.S. Pat. No. 5,716,824; Usman et al., U.S. Pat. No. 5,627,053; Woolf et al., International PCT Publication No. WO 98/13526; Thompson et al., U.S. Ser. No. 60/082,404 which was filed on Apr. 20, 1998; Karpeisky et al., 1998, Tetrahedron Lett., 39, 1131; Earnshaw and Gait, 1998, Biopolymers (Nucleic acid Sciences), 48, 39-55; Verma and Eckstein, 1998, Annu. Rev. Biochem., 67, 99-134; and Burlina et al., 1997, Bioorg. Med. Chem., 5, 1999-2010; all of the references are hereby incorporated in their totality by reference herein). Such publications describe general methods and strategies to determine the location of incorporation of sugar, base and/or phosphate modifications and the like into enzymatic nucleic acids without inhibiting catalysis, and are incorporated by reference herein. In view of such teachings, similar modifications can be used as described herein to modify the nucleic acid molecules of the instant invention.

While chemical modification of oligonucleotide internucleotide linkages with phosphorothioate, phosphorothioate, and/or 5′-methylphosphonate linkages improves stability, too many of these modifications can cause some toxicity. Therefore when designing nucleic acid molecules the amount of these internucleotide linkages should be minimized. The reduction in the concentration of these linkages should lower toxicity resulting in increased efficacy and higher specificity of these molecules.

Nucleic acid molecules having chemical modifications that maintain or enhance activity are provided. Such nucleic acid is also generally more resistant to nucleases than unmodified nucleic acid. Thus, in a cell and/or in vivo the activity can not be significantly lowered. Therapeutic nucleic acid molecules delivered exogenously should optimally be stable within cells until translation of the target RNA has been inhibited long enough to reduce the levels of the undesirable protein. This period of time varies between hours to days depending upon the disease state. Nucleic acid molecules should be resistant to nucleases in order to function as effective intracellular therapeutic agents. Improvements in the chemical synthesis of RNA and DNA (Wincott et al., 1995 Nucleic Acids Res. 23, 2677; Caruthers et al., 1992, Methods in Enzymology 211,3-19 (incorporated by reference herein) have expanded the ability to modify nucleic acid molecules by introducing nucleotide modifications to enhance their nuclease stability as described above.

Use of the nucleic acid-based molecules of the invention can lead to better treatment of the disease progression by affording the possibility of combination therapies (e.g., multiple antisense or enzymatic nucleic acid molecules targeted to different genes, nucleic acid molecules coupled with known small molecule inhibitors, or intermittent treatment with combinations of molecules (including different motifs) and/or other chemical or biological molecules). The treatment of patients with nucleic acid molecules can also include combinations of different types of nucleic acid molecules.

By “enhanced enzymatic activity” is meant to include activity measured in cells and/or in vivo where the activity is a reflection of both catalytic activity and enzymatic nucleic acid stability. In this invention, the product of these properties is increased or not significantly (less that 10 fold) decreased in vivo compared to an all RNA enzymatic nucleic acid or all DNA enzyme.

In another embodiment, nucleic acid catalysts having chemical modifications which maintain or enhance enzymatic activity is provided. Such nucleic acid is also generally more resistant to nucleases than unmodified nucleic acid. Thus, in a cell and/or in vivo the activity should not be significantly lowered. As exemplified herein, such enzymatic nucleic acids are useful in a cell and/or in vivo even if activity over all is reduced 10 fold (Burgin et al., 1996, Biochemistry, 35, 14090). Such enzymatic nucleic acids herein are said to “maintain” the enzymatic activity of an all RNA enzymatic nucleic acid.

In another aspect the nucleic acid molecules comprise a 5′ and/or a 3′-cap structure.

By “cap structure” is meant chemical modifications, which have been incorporated at either terminus of the oligonucleotide (see for example Wincott et al., WO 97/26270, incorporated by reference herein). These terminal modifications protect the nucleic acid molecule from exonuclease degradation, and can help in delivery and/or localization within a cell. The cap can be present at the 5′-terminus (5′-cap) or at the 3′-terminus (3′-cap) or can be present on both terminus. In non-limiting examples: the 5′-cap is selected from the group consisting of inverted abasic residue (moiety), 4′,5′-methylene nucleotide; 1-(beta-D-erythrofuranosyl) nucleotide, 4′-thio nucleotide, carbocyclic nucleotide; 1,5-anhydrohexitol nucleotide; L-nucleotides; alpha-nucleotides; modified base nucleotide; phosphorodithioate linkage; threo-pentofuranosyl nucleotide; acyclic 3′,4′-seco nucleotide; acyclic 3,4-dihydroxybutyl nucleotide; acyclic 3,5-dihydroxypentyl nucleotide, 3′-3′-inverted nucleotide moiety; 3′-3′-inverted abasic moiety; 3′-2′-inverted nucleotide moiety; 3′-2′-inverted abasic moiety; 1,4-butanediol phosphate; 3′-phosphoramidate; hexylphosphate; aminohexyl phosphate; 3′-phosphate; 3′-phosphorothioate; phosphorodithioate; or bridging or non-bridging methylphosphonate moiety (for more details see Wincott et al., International PCT publication No. WO 97/26270, incorporated by reference herein). In yet another preferred embodiment the 3′-cap is selected from a group comprising, 4′,5′-methylene nucleotide; 1-(beta-D-erythrofuranosyl) nucleotide; 4′-thio nucleotide, carbocyclic nucleotide; 5′-amino-alkyl phosphate; 1,3-diamino-2-propyl phosphate, 3-aminopropyl phosphate; 6-aminohexyl phosphate; 1,2-aminododecyl phosphate; hydroxypropyl phosphate; 1,5-anhydrohexitol nucleotide; L-nucleotide; alpha-nucleotide; modified base nucleotide; phosphorodithioate; threopentofuranosyl nucleotide; acyclic 3′,4′-seco nucleotide; 3,4-dihydroxybutyl nucleotide; 3,5-dihydroxypentyl nucleotide, 5′-5′-inverted nucleotide moiety; 5′-5′-inverted abasic moiety; 5′-phosphoramidate; 5′-phosphorothioate; 1,4-butanediol phosphate; 5′-amino; bridging and/or non-bridging 5′-phosphoramidate, phosphorothioate and/or phosphorodithioate, bridging or non bridging methylphosphonate and 5′-mercapto moieties (for more details see Beaucage and Iyer, 1993, Tetrahedron 49, 1925; incorporated by reference herein). By the term “non-nucleotide” is meant any group or compound which can be incorporated into a nucleic acid chain in the place of one or more nucleotide units, including either sugar and/or phosphate substitutions, and allows the remaining bases to exhibit their enzymatic activity. The group or compound is abasic in that it does not contain a commonly recognized nucleotide base, such as adenosine, guanine, cytosine, uracil or thymine.

An “alkyl” group refers to a saturated aliphatic hydrocarbon, including straight-chain, branched-chain, and cyclic alkyl groups. Preferably, the alkyl group has 1 to 12 carbons. More preferably it is a lower alkyl of from 1 to 7 carbons, more preferably 1 to 4 carbons. The alkyl group can be substituted or unsubstituted. When substituted the substituted group(s) is preferably, hydroxyl, cyano, alkoxy, ═O, ═S, NO₂ or N(CH₃)₂, amino, or SH. The term also includes alkenyl groups which are unsaturated hydrocarbon groups containing at least one carbon-carbon double bond, including straight-chain, branched-chain, and cyclic groups. Preferably, the alkenyl group has 1 to 12 carbons. More preferably it is a lower alkenyl of from 1 to 7 carbons, more preferably 1 to 4 carbons. The alkenyl group can be substituted or unsubstituted. When substituted the substituted group(s) is preferably, hydroxyl, cyano, alkoxy, ═O, ═S, NO₂, halogen, N(CH₃)₂, amino, or SH. The term “alkyl” also includes alkynyl groups which have an unsaturated hydrocarbon group containing at least one carbon-carbon triple bond, including straight-chain, branched-chain, and cyclic groups. Preferably, the alkynyl group has 1 to 12 carbons. More preferably it is a lower alkynyl of from 1 to 7 carbons, more preferably 1 to 4 carbons. The alkynyl group can be substituted or unsubstituted. When substituted the substituted group(s) is preferably, hydroxyl, cyano, alkoxy, ═O, ═S, NO₂ or N(CH₃)₂, amino or SH.

Such alkyl groups can also include amine, aryl, alkylaryl, carbocyclic aryl, heterocyclic aryl, amide and ester groups. An “aryl” group refers to an aromatic group which has at least one ring having a conjugated p electron system and includes carbocyclic aryl, heterocyclic aryl and biaryl groups, all of which can be optionally substituted. The preferred substituent(s) of aryl groups are halogen, trihalomethyl, hydroxyl, SH, OH, cyano, alkoxy, alkyl, alkenyl, alkynyl, and amino groups. An “alkylaryl” group refers to an alkyl group (as described above) covalently joined to an aryl group (as described above). Carbocyclic aryl groups are groups wherein the ring atoms on the aromatic ring are all carbon atoms. The carbon atoms are optionally substituted. Heterocyclic aryl groups are groups having from 1 to 3 heteroatoms as ring atoms in the aromatic ring and the remainder of the ring atoms are carbon atoms. Suitable heteroatoms include oxygen, sulfur, and nitrogen, and include furanyl, thienyl, pyridyl, pyrrolyl, N-lower alkyl pyrrolo, pyrimidyl, pyrazinyl, imidazolyl and the like, all optionally substituted. An “amide” refers to an —C(O)—NH—R, where R is either alkyl, aryl, alkylaryl or hydrogen. An “ester” refers to an —C(O)—OR′, where R is either alkyl, aryl, alkylaryl or hydrogen.

By “nucleotide” as used herein is as recognized in the art to include natural bases (standard), and modified bases well known in the art. Such bases are generally located at the 1′ position of a nucleotide sugar moiety. Nucleotides generally comprise a base, sugar and a phosphate group. The nucleotides can be unmodified or modified at the sugar, phosphate and/or base moiety, (also referred to interchangeably as nucleotide analogs, modified nucleotides, non-natural nucleotides, non-standard nucleotides and other; see, for example, Usman and McSwiggen, supra; Eckstein et al, International PCT Publication No. WO 92/07065; Usman et al., International PCT Publication No. WO 93/15187; Ulhlman & Peyman, supra all are hereby incorporated by reference herein). There are several examples of modified nucleic acid bases known in the art as summarized by Limbach et al, 1994, Nucleic Acids Res. 22, 2183. Some of the non-limiting examples of base modifications that can be introduced into nucleic acid molecules include, inosine, purine, pyridin-4-one, pyridin-2-one, phenyl, pseudouracil, 2, 4, 6-trimethoxy benzene, 3-methyl uracil, dihydrouridine, naphthyl, aminophenyl, 5-alkylcytidines (e.g., 5-methylcytidine), 5-alkyluridines (e.g., ribothymidine), 5-halouridine (e.g., 5-bromouridine) or 6-azapyrimidines or 6-alkylpyrimidines (e.g. 6-methyluridine), propyne, and others (Burgin et al., 1996, Biochemistry, 35, 14090; Uhlman & Peyman, supra). By “modified bases” in this aspect is meant nucleotide bases other than adenine, guanine, cytosine and uracil at 1′ position or their equivalents; such bases can be used at any position, for example, within the catalytic core of an enzymatic nucleic acid molecule and/or in the substrate-binding regions of the nucleic acid molecule.

In one embodiment, the invention features modified nucleic acids with phosphate backbone modifications comprising one or more phosphorothioate, phosphorodithioate, methylphosphonate, morpholino, amidate carbamate, carboxymethyl, acetamidate, polyamide, sulfonate, sulfonamide, sulfamate, formacetal, thioformacetal, and/or alkylsilyl, substitutions. For a review of oligonucleotide backbone modifications see Hunziker and Leumann, 1995, Nucleic Acid Analogues: Synthesis and Properties, in Modern Synthetic Methods, VCH, 331-417, and Mesmaeker et al., 1994, Novel Backbone Replacements for Oligonucleotides, in Carbohydrate Modifications in Antisense Research, ACS, 24-39. These references are hereby incorporated by reference herein.

By “abasic” or “abasic moiety” is meant sugar moieties lacking a base or having other chemical groups in place of a base at the 1′ position, (see Wincott et al., International PCT publication No. WO 97/26270).

By “ribofuranose sugar moiety” is meant a naturally occurring or chemically modified component of a ribofuranose sugar.

By “bridging phosphate moiety” is meant a naturally occurring or chemically modified bridging component of a phosphate group.

By “non-bridging phosphate moiety” is meant a naturally occurring or chemically modified non-bridging component of a phosphate group.

By “unmodified nucleoside” is meant one of the bases adenine, cytosine, guanine, thymine, uracil joined to the 1′ carbon of β-D-ribo-furanose.

By “modified nucleoside” is meant any nucleotide base which contains a modification in the chemical structure of an unmodified nucleotide base, sugar and/or phosphate.

In connection with 2′-modified nucleotides as described for the present invention, by “amino” is meant 2′-NH₂ or 2′-O—NH₂, which can be modified or unmodified. Such modified groups are described, for example, in Eckstein et al., U.S. Pat. No. 5,672,695 and Matulic-Adamic et al., WO 98/28317, respectively, which are both incorporated by reference in their entireties.

Various modifications to nucleic acid (e.g., antisense and enzymatic nucleic acid) structure can be made to enhance the utility of these molecules, including, for example, modifications that enhance shelf-life, half-life in vitro, stability, and ease of introduction of such oligonucleotides to the target site, e.g., to enhance penetration of cellular membranes, and confer the ability to recognize and bind to targeted cells.

Use of these molecules can lead to better treatment of the disease progression by affording the possibility of combination therapies (e.g., multiple enzymatic nucleic acids targeted to different genes, enzymatic nucleic acids coupled with known small molecule inhibitors, or intermittent treatment with combinations of enzymatic nucleic acids (including different enzymatic nucleic acid motifs) and/or other chemical or biological molecules. The treatment of patients with nucleic acid molecules can also include combinations of different types of nucleic acid molecules. Therapies can be devised which include a mixture of enzymatic nucleic acids (including different enzymatic nucleic acid motifs), antisense and/or 2-5A chimera molecules to one or more targets to alleviate symptoms of a disease.

Administration of Nucleic Acid Molecules

Sullivan et al., PCT WO 94/02595, describes the general methods for delivery of enzymatic nucleic acid molecules. Nucleic acid molecules can be administered to cells by a variety of methods known to those familiar to the art, including, but not restricted to, encapsulation in liposomes, by iontophoresis, or by incorporation into other vehicles, such as hydrogels, cyclodextrins, biodegradable nanocapsules, and bioadhesive microspheres. For some indications, enzymatic nucleic acids can be directly delivered ex vivo to cells or tissues with or without the aforementioned vehicles. Alternatively, the RNA/vehicle combination is locally delivered by direct injection or by use of a catheter, infusion pump, stent or other delivery devices such as Alzet® pumps, Medipad® devices. Other routes of delivery include, but are not limited to, intravascular, intramuscular, subcutaneous or joint injection, aerosol inhalation, oral (tablet or pill form), topical, systemic, ocular, intraperitoneal and/or intrathecal delivery. More detailed descriptions of enzymatic nucleic acid delivery and administration are provided in Sullivan et al., supra and Draper et a., PCT WO93/23569 which have been incorporated by reference herein.

The molecules of the instant invention can be used as pharmaceutical agents. Pharmaceutical agents prevent, inhibit the occurrence, or treat (alleviate a symptom to some extent, preferably all of the symptoms) of a disease state in a patient.

The negatively charged polynucleotides of the invention can be administered (e.g., RNA, DNA or protein) and introduced into a patient by any standard means known in the art, with or without stabilizers, buffers, and the like, to form a pharmaceutical composition. When it is desired to use a lipid or liposome delivery mechanism, standard protocols for formulation can be followed. The compositions of the present invention can also be formulated and used as tablets, capsules or elixirs for oral administration; suppositories for rectal administration; sterile solutions; suspensions for injectable administration; and the like.

The present invention also includes pharmaceutically acceptable formulations of the compounds described. These formulations include salts of the above compounds, e.g., acid addition salts, for example, salts of hydrochloric, hydrobromic, acetic acid, and benzene sulfonic acid.

A pharmacological composition or formulation refers to a composition or formulation in a form suitable for administration, e.g., systemic administration, into a cell or patient, preferably a human. Suitable forms, in part, depend upon the use or the route of entry, for example oral, transdermal, or by injection. Such forms should not prevent the composition or formulation to reach a target cell (i.e., a cell to which the negatively charged polymer is desired to be delivered to). For example, pharmacological compositions injected into the blood stream should be soluble. Other factors are known in the art, and include considerations such as toxicity and forms which prevent the composition or formulation from exerting its effect.

By “systemic administration” is meant in vivo systemic absorption or accumulation of drugs in the blood stream followed by distribution throughout the entire body. Administration routes which lead to systemic absorption include, without limitations: intravenous, subcutaneous, intraperitoneal, inhalation, oral, intrapulmonary and intramuscular. Each of these administration routes expose the desired negatively charged polymers, e.g., nucleic acids, to an accessible diseased tissue. The rate of entry of a drug into the circulation has been shown to be a function of molecular weight or size. The use of a liposome or other drug carrier comprising the compounds of the instant invention can potentially localize the drug, for example, in certain tissue types, such as the tissues of the reticular endothelial system (RES). A liposome formulation which facilitates the association of drug with the surface of cells, such as, lymphocytes and macrophages is also useful. This approach can provide enhanced delivery of the drug to target cells by taking advantage of the specificity of macrophage and lymphocyte immune recognition of abnormal cells, such as HCV infected liver cells.

In one embodiment, the invention features the use of a composition comprising surface-modified liposomes containing poly (ethylene glycol) lipids (PEG-modified, or long-circulating liposomes or stealth liposomes). These formulations offer a method for increasing the accumulation of drugs in target tissues. This class of drug carriers resists opsonization and elimination by the mononuclear phagocytic system (MPS or RES), thereby enabling longer blood circulation times and enhanced tissue exposure for the encapsulated drug (Lasic et al. Chem. Rev. 1995, 95, 2601-2627; Ishiwata et al., Chem. Pharm. Bull. 1995, 43, 1005-1011). Such liposomes have been shown to accumulate selectively in tumors, presumably by extravasation and capture in the neovascularized target tissues (Lasic et al., Science 1995, 267, 1275-1276; Oku et al., 1995, Biochim. Biophys. Acta, 1238, 86-90). The long-circulating liposomes enhance the pharmacokinetics and pharmacodynamics of DNA and RNA, particularly compared to conventional cationic liposomes which are known to accumulate in tissues of the MPS (Liu et al., J. Biol. Chem. 1995, 42, 24864-24870; Choi et al, International PCT Publication No. WO 96/10391; Ansell et al., International PCT Publication No. WO 96/10390; Holland et al., International PCT Publication No. WO 96/10392; all of the se are incorporated by reference herein). All of these references are incorporated by reference herein.

In addition, other cationic molecules can also be utilized to deliver the molecules of the present invention. For example, enzymatic nucleic acid molecules can be conjugated to glycosylated poly(L-lysine) which has been shown to enhance localization of antisense oligonucleotides into the liver (Nakazono et al., 1996, Hepatology 23, 1297-1303; Nahato et al., 1997, Biochem Pharm. 53, 887-895). Glycosylated poly(L-lysine) can be covertly attached to the enzymatic nucleic acid or be bound to enzymatic nucleic acid through electrostatic interaction.

The present invention also includes compositions prepared for storage or administration which include a pharmaceutically effective amount of the desired compounds in a pharmaceutically acceptable carrier or diluent. Acceptable carriers or diluents for therapeutic use are well known in the pharmaceutical art, and are described, for example, in Remington's Pharmaceutical Sciences, Mack Publishing Co. (A. R. Gennaro edit. 1985) hereby incorporated by reference herein. For example, preservatives, stabilizers, dyes and flavoring agents can be provided. Id. at 1449. These include sodium benzoate, sorbic acid and esters of p-hydroxybenzoic acid. In addition, antioxidants and suspending agents can be used.

A pharmaceutically effective dose is that dose required to prevent, inhibit the occurrence, or treat (alleviate a symptom to some extent, preferably all of the symptoms) a disease state. The pharmaceutically effective dose depends on the type of disease, the composition used, the route of administration, the type of mammal being treated, the physical characteristics of the specific mammal under consideration, concurrent medication, and other factors which those skilled in the medical arts will recognize. Generally, an amount between 0.1 mg/kg and 100 mg/kg body weight/day of active ingredients is administered dependent upon potency of the negatively charged polymer.

The nucleic acid molecules of the present invention can also be administered to a patient in combination with other therapeutic compounds to increase the overall therapeutic effect. The use of multiple compounds to treat an indication can increase the beneficial effects while reducing the presence of side effects.

Alternatively, the enzymatic nucleic acid molecules of the instant invention can be expressed within cells from eukaryotic promoters (e.g., Izant and Weintraub, 1985 Science 229, 345; McGarry and Lindquist, 1986 Proc. Natl. Acad. Sci. USA 83, 399; Scanlon et al., 1991, Proc. Natl. Acad. Sci. USA, 88, 10591-5; Kashani-Sabet et al., 1992 Antisense Res. Dev., 2, 3-15; Dropulic et al., 1992 J. Virol, 66, 1432-41; Weerasinghe et al., 1991 J. Virol, 65, 5531-4; Ojwang et al., 1992 Proc. Natl. Acad. Sci. USA 89, 10802-6; Chen et al., 1992 Nucleic Acids Res., 20, 4581-9; Sarver et al., 1990 Science 247, 1222-1225; Thompson et al., 1995 Nucleic Acids Res. 23, 2259; Good et al., 1997, Gene Therapy, 4, 45; all of the references are hereby incorporated in their totality by reference herein). Those skilled in the art realize that any nucleic acid can be expressed in eukaryotic cells from the appropriate DNA/RNA vector. The activity of such nucleic acids can be augmented by their release from the primary transcript by an enzymatic nucleic acid (Draper et al., PCT WO 93/23569, and Sullivan et al., PCT WO 94/02595; Ohkawa et al., 1992 Nucleic Acids Symp. Ser., 27, 15-6; Taira et al., 1991, Nucleic Acids Res., 19, 5125-30; Ventura et al., 1993 Nucleic Acids Res., 21, 3249-55; Chowrira et al., 1994 J. Biol. Chem. 269, 25856; all of the references are hereby incorporated in their totality by reference herein).

In another aspect of the invention, nucleic acid molecules that cleave target molecules are expressed from transcription units (see for example Couture et al., 1996, TIG., 12, 510) inserted into DNA or RNA vectors. The recombinant vectors are preferably DNA plasmids or viral vectors. Nucleic acid molecule expressing viral vectors can be constructed based on, but not limited to, adeno-associated virus, retrovirus, adenovirus, or alphavirus. Preferably, the recombinant vectors capable of expressing the nucleic acid molecules are delivered as described above, and persist in target cells. Alternatively, viral vectors can be used that provide for transient expression of nucleic acid molecules. Such vectors can be repeatedly administered as necessary. Once expressed, the nucleic acid molecules cleave the target mRNA. The active nucleic acid molecule can contain an enzymatic center or core equivalent to those in the examples, and binding arms able to bind target nucleic acid molecules such that cleavage at the target site occurs. Other sequences can be present which do not interfere with such cleavage. Delivery of enzymatic nucleic acid molecule expressing vectors could be systemic, such as by intravenous or intramuscular administration, by administration to target cells ex-planted from the patient followed by reintroduction into the patient, or by any other means that would allow for introduction into the desired target cell (for a review see Couture et al., 1996, TIG., 12, 510).

In one aspect the invention features an expression vector comprising nucleic acid sequence encoding at least one of the nucleic acid catalyst of the instant invention is disclosed. The nucleic acid sequence encoding the nucleic acid catalyst of the instant invention is operable linked in a manner that allows expression of that nucleic acid molecule.

In another aspect the invention features an expression vector comprising: a) a transcription initiation region (e.g., eukaryotic pol I, II or III initiation region); b) a transcription termination region (e.g., eukaryotic pol I, II or III termination region); c) a nucleic acid sequence encoding at least one of the nucleic acid catalyst of the instant invention; and wherein said sequence is operably linked to said initiation region and said termination region, in a manner which allows expression and/or delivery of said nucleic acid molecule. The vector can optionally include an open reading frame (ORF) for a protein operably linked on the 5′ side or the 3′-side of the sequence encoding the nucleic acid catalyst of the invention; and/or an intron (intervening sequences).

Transcription of the nucleic acid molecule sequences are driven from a promoter for eukaryotic RNA polymerase I (pol I), RNA polymerase II (pol II), or RNA polymerase III (pol III). Transcripts from pol II or pol III promoters are expressed at high levels in all cells; the levels of a given pol II promoter in a given cell type depends on the nature of the gene regulatory sequences (enhancers, silencers, etc.) present nearby. Prokaryotic RNA polymerase promoters are also used, providing that the prokaryotic RNA polymerase enzyme is expressed in the appropriate cells (Elroy-Stein and Moss, 1990, Proc. Natl. Acad. Sci. U S A, 87, 6743-7; GAO and Huang 1993, Nucleic Acids Res., 21, 2867-72; Lieber et al., 1993, Methods Enzymol., 217, 47-66; Shout et al., 1990, Mol. Cell. Biol., 10, 4529-37). All of these references are incorporated by reference herein. Several investigators have demonstrated that nucleic acid molecules, such as enzymatic nucleic acids expressed from such promoters can function in mammalian cells (e.g. Kashani-Sabet et al., 1992, Antisense Res. Dev., 2, 3-15; Ojwang et al., 1992, Proc. Natl. Acad. Sci. U S A, 89, 10802-6; Chen et al., 1992, Nucleic Acids Res., 20, 4581-9; Yu et al., 1993, Proc. Natl. Acad. Sci. USA, 90, 6340-4; L'Huillier et al., 1992, EMBO J, 11, 4411-8; Lisziewicz et al., 1993, Proc. Natl. Acad. Sci. U.S. A, 90, 8000-4; Thompson et al., 1995, Nucleic Acids Res., 23, 2259; Sullenger & Cech, 1993, Science, 262, 1566). More specifically, transcription units such as the ones derived from genes encoding U6 small nuclear (snRNA), transfer RNA (tRNA) and adenovirus VA RNA are useful in generating high concentrations of desired RNA molecules such as enzymatic nucleic acids in cells (Thompson et al., supra; Couture and Stinchcomb, 1996, supra; Noonberg et al., 1994, Nucleic Acid Res., 22, 2830; Noonberg et al., U.S. Pat. No. 5,624,803; Good et al., 1997, Gene Ther., 4, 45; Beigelman et al., International PCT Publication No. WO 96/18736; all of these publications are incorporated by reference herein. The above enzymatic nucleic acid molecule transcription units can be incorporated into a variety of vectors for introduction into mammalian cells, including but not restricted to, plasmid DNA vectors, viral DNA vectors (such as adenovirus or adeno-associated virus vectors), or viral RNA vectors (such as retroviral or alphavirus vectors) (for a review see Couture and Stinchcomb, 1996, supra).

In another aspect the invention features an expression vector comprising nucleic acid sequence encoding at least one of the nucleic acid molecules of the invention, in a manner which allows expression of that nucleic acid molecule. The expression vector comprises in one embodiment; a) a transcription initiation region; b) a transcription termination region; c) a nucleic acid sequence encoding at least one said nucleic acid molecule; and wherein said sequence is operably linked to said initiation region and said termination region, in a manner which allows expression and/or delivery of said nucleic acid molecule. In another preferred embodiment the expression vector comprises: a) a transcription initiation region; b) a transcription termination region; c) an open reading frame; d) a nucleic acid sequence encoding at least one said nucleic acid molecule, wherein said sequence is operably linked to the 3′-end of said open reading frame; and wherein said sequence is operably linked to said initiation region, said open reading frame and said termination region, in a manner which allows expression and/or delivery of said nucleic acid molecule. In yet another embodiment the expression vector comprises: a) a transcription initiation region; b) a transcription termination region; c) an intron; d) a nucleic acid sequence encoding at least one said nucleic acid molecule; and wherein said sequence is operably linked to said initiation region, said intron and said termination region, in a manner which allows expression and/or delivery of said nucleic acid molecule. In another embodiment, the expression vector comprises: a) a transcription initiation region; b) a transcription termination region; c) an intron; d) an open reading frame; e) a nucleic acid sequence encoding at least one said nucleic acid molecule, wherein said sequence is operably linked to the 3′-end of said open reading frame; and wherein said sequence is operably linked to said initiation region, said intron, said open reading frame and said termination region, in a manner which allows expression and/or delivery of said nucleic acid molecule.

Interferons

Type I interferons (IFN) are a class of natural cytokines that includes a family of greater than 25 IFN-α (Pesta, 1986, Methods Enzymol. 119, 3-14) as well as IFN-β, and IFN-ω. Although evolutionarily derived from the same gene (Diaz et al., 1994, Genomics 22, 540-552), there are many differences in the primary sequence of these molecules, implying an evolutionary divergence in biologic activity. All type I IFN share a common pattern of biologic effects that begin with binding of the IFN to the cell surface receptor (Pfeffer & Strulovici, 1992, Transmembrane secondary messengers for IFN-α/β. In: Interferon. Principles and Medical Applications., S. Baron, D. H. Coopenhaver, F. Dianzani, W. R. Fleischmann Jr., T. K. Hughes Jr., G. R. Kimpel, D. W. Niesel, G. J. Stanton, and S. K. Tyring, eds. 151-160). Binding is followed by activation of tyrosine kinases, including the Janus tyrosine kinases and the STAT proteins, which leads to the production of several IFN-stimulated gene products (Johnson et al., 1994, Sci. Am. 270, 68-75). The IFN-stimulated gene products are responsible for the pleotropic biologic effects of type I IFN, including antiviral, antiproliferative, and immunomodulatory effects, cytokine induction, and HLA class I and class II regulation (Pestka et al., 1987, Annu. Rev. Biochem 56, 727). Examples of IFN-stimulated gene products include 2-5-oligoadenylate synthetase (2-5 OAS), β₂-microglobulin, neopterin, p68 kinases, and the Mx protein (Chebath & Revel, 1992, The 2-5 A system: 2-5 A synthetase, isospecies and functions. In: Interferon. Principles and Medical Applications. S. Baron, D. H. Coopenhaver, F. Dianzani, W. R. Jr. Fleischmann, T. K. Jr Hughes, G. R. Kimpel, D. W. Niesel, G. J. Stanton, and S. K. Tyring, eds., pp. 225-236; Samuel, 1992, The RNA-dependent P1/eIF-2α protein kinase. In: Interferon. Principles and Medical Applications. S. Baron, D. H. Coopenhaver, F. Dianzani, W. R. Fleischmann Jr., T. K. Hughes Jr., G. R. Kimpel, D. W. Niesel, G. H. Stanton, and S. K. Tyring, eds. 237-250; Horisberger, 1992, MX protein: function and Mechanism of Action. In: Interferon. Principles and Medical Applications. S. Baron, D. H. Coopenhaver, F. Dianzani, W. R. Fleischmann Jr., T. K. Hughes Jr., G. R. Kimpel, D. W. Niesel, G. H. Stanton, and S. K. Tyring, eds. 215-224). Although all type I IFN have similar biologic effects, not all the activities are shared by each type I IFN, and, in many cases, the extent of activity varies quite substantially for each IFN subtype (Fish et al, 1989, J. Interferon Res. 9, 97-114; Ozes et al., 1992, J. Interferon Res. 12, 55-59). More specifically, investigations into the properties of different subtypes of IFN-α and molecular hybrids of IFN-α have shown differences in pharmacologic properties (Rubinstein, 1987, J. Interferon Res. 7, 545-551). These pharmacologic differences can arise from as few as three amino acid residue changes (Lee et al., 1982, Cancer Res. 42, 1312-1316).

Eighty-five to 166 amino acids are conserved in the known IFN-α subtypes. Excluding the IFN-α pseudogenes, there are approximately 25 known distinct IFN-α subtypes. Pairwise comparisons of these nonallelic subtypes show primary sequence differences ranging from 2% to 23%. In addition to the naturally occurring IFNs, a non-natural recombinant type I interferon known as consensus interferon (CIFN) has been synthesized as a therapeutic compound (Tong et al., 1997, Hepatology 26, 747-754).

Interferon is currently in use for at least 12 different indications including infectious and autoimmune diseases and cancer (Borden, 1992, N. Engl. J. Med. 326, 1491-1492). For autoimmune diseases IFN has been utilized for treatment of rheumatoid arthritis, multiple sclerosis, and Crohn's disease. For treatment of cancer IFN has been used alone or in combination with a number of different compounds. Specific types of cancers for which IFN has been used include squamous cell carcinomas, melanomas, hypernephromas, hemangiomas, hairy cell leukemia, and Kaposi's sarcoma. In the treatment of infectious diseases, IFNs increase the phagocytic activity of macrophages and cytotoxicity of lymphocytes and inhibits the propagation of cellular pathogens. Specific indications for which IFN has been used as treatment include: hepatitis B, human papillomavirus types 6 and 11 (i.e. genital warts) (Leventhal et al., 1991, N Engl J Med 325, 613-617), chronic granulomatous disease, and hepatitis C virus.

Numerous well controlled clinical trials using IFN-alpha in the treatment of chronic HCV infection have demonstrated that treatment three times a week results in lowering of serum ALT values in approximately 50% (range 40% to 70%) of patients by the end of 6 months of therapy (Davis et al., 1989, The new England Journal of Medicine 321, 1501-1506; Marcellin et al., 1991, Hepatology 13, 393-397; Tong et al., 1997, Hepatology 26, 747-754; Tong et al., Hepatology 26, 1640-1645). However, following cessation of interferon treatment, approximately 50% of the responding patients relapsed, resulting in a “durable” response rate as assessed by normalization of serum ALT concentrations of approximately 20 to 25%. In addition, studies that have examined six months of type 1 interferon therapy using changes in HCV RNA values as a clinical endpoint have demonstrated that up to 35% of patients will have a loss of HCV RNA by the end of therapy (Tong et al., 1997, supra). However, as with the ALT endpoint, about 50% of the patients relapse six months following cessation of therapy resulting in a durable virologic response of only 12% (23). Studies that have examined 48 weeks of therapy have demonstrated that the sustained virological response is up to 25%.

Pegylated interferons, i.e. interferons conjugated with polyethylene glycol (PEG), have demonstrated improved characteristics over interferon. Advantages incurred by PEG conjugation can include an improved pharmacokinetic profile compared to interferons lacking PEG, thus imparting more convenient dosing regimes, improved tolerance, and improved antiviral efficacy. Such improvements have been demonstrated in clinical studies of both polyethylene glycol interferon alfa-2a (PEGASYS, Roche) and polyethylene glycol interferon alfa-2b (VIRAFERON PEG, PEG-INTRON, Enzon/Schering Plough).

Enzymatic nucleic acid molecules in combination with interferons and polyethylene glycol interferons have the potential to improve the effectiveness of treatment of HCV or any of the other indications discussed above. Enzymatic nucleic acid molecules targeting RNAs associated with diseases such as infectious diseases, autoimmune diseases, and cancer, can be used individually or in combination with other therapies such as interferons and polyethylene glycol interferons and to achieve enhanced efficacy.

### EXAMPLES

The following are non-limiting examples showing the selection, isolation, synthesis and activity of enzymatic nucleic acids of the instant invention.

The following examples demonstrate the use of enzymatic nucleic acid molecules that cleave HCV RNA. The methods described herein represent a scheme by which nucleic acid molecules can be derived that cleave other RNA targets required for HCV replication.

### Example 1

### Identification of Potential Enzymatic Nucleic Acid Molecules Cleavage Sites in HCV RNA

The sequence of HCV RNA was screened for accessible sites using a computer folding algorithm. Regions of the mRNA that did not form secondary folding structures and contained potential enzymatic nucleic acid cleavage sites were identified. The sequences of these cleavage sites are shown in Tables III, IV, V and VIII.

### Example 2

### Selection of Enzymatic Nucleic Acid Molecules Cleavage Sites in HCV RNA

Enzymatic nucleic acid target sites were chosen by analyzing sequences of Human HCV (Genbank accession Nos: D11168, D50483.1, L38318 and S82227) and prioritizing the sites on the basis of folding. Enzymatic nucleic acid molecules are designed that could bind each target and are individually analyzed by computer folding (Christoffersen et al., 1994 J. Mol. Struc. Theochem, 311, 273; Jaeger et al., 1989, Proc. Natl. Acad. Sci. USA, 86, 7706) to assess whether the enzymatic nucleic acid molecules sequences fold into the appropriate secondary structure. Those enzymatic nucleic acid molecules with unfavorable intramolecular interactions between the binding arms and the catalytic core can be eliminated from consideration. As noted below, varying binding arm lengths can be chosen to optimize activity. Generally, at least 4 bases on each arm are able to bind to, or otherwise interact with, the target RNA.

### Example 3

### Chemical Synthesis and Purification of Enzymatic Nucleic Acids

Enzymatic nucleic acid molecules are designed to anneal to various sites in the RNA message. The binding arms of the enzymatic nucleic acid molecules are complementary to the target site sequences described above. The enzymatic nucleic acid molecules can be chemically synthesized using, for example, RNA syntheses such as those described above and those described in Usman et al., (1987 J. Am. Chem. Soc., 109, 7845), Scaringe et al., (1990 Nucleic Acids Res., 18, 5433) and Wincott et al., supra. Such methods make use of common nucleic acid protecting and coupling groups, such as dimethoxytrityl at the 5′-end, and phosphoramidites at the 3′-end. The average stepwise coupling yields are typically >98%. Enzymatic nucleic acid molecules can be modified to enhance stability by modification with nuclease resistant groups, for example, 2′-amino, 2′-C-allyl, 2′-flouro, 2′-O-methyl, 2′-H (for a review see Usman and Cedergren, 1992 TIBS 17, 34).

Enzymatic nucleic acid molecules can also be synthesized from DNA templates using bacteriophage T7 RNA polymerase (Milligan and Uhlenbeck, 1989, Methods Enzymol. 180, 51). Enzymatic nucleic acid molecules can be purified by gel electrophoresis using known methods, or can be purified by high pressure liquid chromatography (HPLC; See Wincott et al., supra; the totality of which is hereby incorporated herein by reference), and are resuspended in water. The sequences of chemically synthesized enzymatic nucleic acid constructs are shown below in Tables V and VI. The antisense nucleic acid molecules shown in Table VII were chemically synthesized.

Inactive enzymatic nucleic acid molecules, for example inactive hammerhead enzymatic nucleic acids, can be synthesized by substituting the order of G5A6 and substituting a U for A14 (numbering from Hertel et al., 1992 Nucleic Acids Res., 20, 3252).

### Example 4

### Enzymatic Nucleic Acid Cleavage of HCV RNA Target in vitro

Enzymatic nucleic acid molecules targeted to the HCV are designed and synthesized as described above. These enzymatic nucleic acid molecules can be tested for cleavage activity in vitro, for example, using the following procedure. The target sequences and the nucleotide location within the HCV are given in Tables V and VIII.

Cleavage Reactions:

Full-length or partially full-length, internally-labeled target RNA for enzymatic nucleic acid molecule cleavage assay is prepared by in vitro transcription in the presence of [α-32p] CTP, passed over a G 50 Sephadex column by spin chromatography and used as substrate RNA without further purification. Alternately, substrates are 5′-³²P-end labeled using T4 polynucleotide kinase enzyme. Assays are performed by pre-wanning a 2× concentration of purified enzymatic nucleic acid molecule in enzymatic nucleic acid molecule cleavage buffer (50 mM Tris-HCl, pH 7.5 at 37° C., 10 mM MgCl₂) and the cleavage reaction was initiated by adding the 2× enzymatic nucleic acid molecule mix to an equal volume of substrate RNA (maximum of 1-5 nM) that was also pre-warmed in cleavage buffer. As an initial screen, assays are carried out for 1 hour at 37° C. using a final concentration of either 40 nM or 1 mM enzymatic nucleic acid molecule, i.e., enzymatic nucleic acid molecule excess. The reaction is quenched by the addition of an equal volume of 95% formamide, 20 mM EDTA, 0.05% bromophenol blue and 0.05% xylene cyanol after which the sample is heated to 95° C. for 2 minutes, quick chilled and loaded onto a denaturing polyacrylamide gel. Substrate RNA and the specific RNA cleavage products generated by enzymatic nucleic acid molecule cleavage are visualized on an autoradiograph of the gel. The percentage of cleavage is determined by Phosphor Imager® quantitation of bands representing the intact substrate and the cleavage products.

Alternatively, enzymatic nucleic acid molecules and substrates were synthesized in 96-well format using 0.2 μmol scale. Substrates were 5′-³²p labeled and gel purified using 7.5% polyacrylamide gels, and eluting into water. Assays were done by combining trace substrate with 500 nM enzymatic nucleic acid or greater, and initiated by adding final concentrations of 40 mM Mg⁺², and 50 mM Tris-Cl pH 8.0. For each enzymatic nucleic acid/substrate combination a control reaction was done to ensure cleavage was not the result of non-specific substrate degradation. A single three hour time point was taken and run on a 15% polyacrylamide gel to asses cleavage activity. Gels were dried and scanned using a Molecular Dynamics Phosphorimager and quantified using Molecular Dynamics ImageQuant software. Percent cleaved was determined by dividing values for cleaved substrate bands by full-length (uncleaved) values plus cleaved values and multiplying by 100 (%cleaved=[C/(U+C)]*100). In vitro cleavage data of enzymatic nucleic acid molecules targeting plus and minus strand HCV RNA is shown in Table VIII.

### Example 5

### Inhibition of Luciferase Activity Using HCV Targeting Enzymatic Nucleic Acids in OST7 Cells

The capability of enzymatic nucleic acids to inhibit HCV RNA intracellularly was tested using a dual reporter system that utilizes both firefly and Renilla luciferase (FIG. 6). The enzymatic nucleic acids targeted to the 5′ HCV UTR region, which when cleaved, prevents the translation of the transcript into luciferase.

Synthesis of Stabilized Enzymatic Nucleic Acids

Enzymatic nucleic acids were designed to target 15 sites within the 5′UTR of the HCV RNA (FIG. 7) and synthesized as previously described, except that all enzymatic nucleic acids contain two 2′-amino uridines. Enzymatic nucleic acid and paired control sequences for targeted sites used in various examples herein are shown in Table VI.

Reporter Plasmids

The T7/HCV/firefly luciferase plasmid (HCVT7C₁₋₃₄₁, genotype 1a) was graciously provided by Aleem Siddiqui (University of Colorado Health Sciences Center, Denver, Colo.). The T7/HCV/firefly luciferase plasmid contains a T7 bacteriophage promoter upstream of the HCV 5′UTR (nucleotides 1-341)/firefly luciferase fusion DNA. The Renilla luciferase control plasmid (pRLSV40) was purchased from PROMEGA.

Luciferase Assay

Dual luciferase assays were carried out according to the manufacturer's instructions (PROMEGA) at 4 hours after co-transfection of reporter plasmids and enzymatic nucleic acids. All data is shown as the average ratio of HCV/firefly luciferase luminescence over Renilla luciferase luminescence as determined by triplicate samples ±SD.

Cell Culture and Transfections

OST7 cells were maintained in Dulbecco's modified Eagle's medium (GIBCO BRL) supplemented with 10% fetal calf serum, L-glutamine (2 mM) and penicillin/streptomycin. For transfections, OST7 cells were seeded in black-walled 96-well plates (Packard) at a density of 12,500 cells/well and incubated at 37° C. under 5% CO₂ for 24 hours. Co-transfection of target reporter HCVT7C (0.8 μg/mL), control reporter pRLSV40, (1.2μg/mL) and enzymatic nucleic acid, (50-200 nM) was achieved by the following method: a 5× mixture of HCVT7C (4 μg/mL), pRLSV40 (6 μg/mL) enzymatic nucleic acid (250-1000 nM) and cationic lipid (28.5 μg/mL) was made in 150 μL of OPTI-MEM (GIBCO BRL) minus serum. Reporter/enzymatic nucleic acid/lipid complexes were allowed to form for 20 min at 37° C. under 5% CO₂. Medium was aspirated from OST7 cells and replaced with 120 μL of OPTI-MEM (GIBCO BRL) minus serum, immediately followed by the addition of 30 μL of 5× reporter/enzymatic nucleic acid/lipid complexes. Cells were incubated with complexes for 4 hours at 37° C. under 5% CO₂.

IC50 Determinations for Dose Response Curves

Apparent IC₅₀ values were calculated by linear interpolation. The apparent IC₅₀ is 1/2 the maximal response between the two consecutive points in which approximately 50% inhibition of HCV/luciferase expression is observed on the dose curve.

Quantitation of RNA Samples

Total RNA from transfected cells was purified using the Qiagen RNeasy 96 procedure including a DNase I treatment according to the manufacturer's instructions. Real time RT-PCR (Taqman assay) was performed on purified RNA samples using separate primer/probe sets specific for either firefly or Renilla luciferase RNA. Firefly luciferase primers and probe were upper (5′-CGGTCGGTAAAGTTGTTCCATT-3′) (SEQ ID NO. 9690), lower (5′-CCTCTGACACATAATTCGCCTCT-3′) (SEQ ID NO. 9691), and probe (5′-FAM-TGAAGCGAAGGTTGTGGATCTGGATACC-TAMRA-3′) (SEQ ID NO 9692), and Renilla luciferase primers and probe were upper (5′-GTTTATTGAATCGGACCCAGGAT-3′) (SEQ ID NO. 9693), lower (5′-AGGTGCATCTTCTTGCGAAAA-3′) (SEQ ID NO. 9694), and probe (5′-FAM-CTTTTCCAATGCTATTGTTGAAGGTGCCAA-3′) (SEQ ID NO. 9694)-TAMRA, both sets of primers and probes were purchased from Integrated DNA Technologies. RNA levels were determined from a standard curve of amplified RNA purified from a large-scale transfection. RT minus controls established that RNA signals were generated from RNA and not residual plasmid DNA. RT-PCR conditions were: 30 min at 48° C., 10 min at 95° C., followed by 40 cycles of 15 sec at 95° C. and 1 min at 60° C. Reactions were performed on an ABI Prism 7700 sequence detector. Levels of firefly luciferase RNA were normalized to the level of Renilla luciferase RNA present in the same sample. Results are shown as the average of triplicate treatments ±SD.

### Example 6

### Inhibition of HCV 5′UTR-luciferase Expression by Synthetic Stabilized Enzymatic Nucleic Acids

The primary sequence of the HCV 5′UTR and characteristic secondary structure (FIG. 7) is highly conserved across all HCV genotypes, thus making it a very attractive target for enzymatic nucleic acid-mediated cleavage. Enzymatic hammerhead nucleic acids, as a generally shown in FIG. 8 and Table VI (RPI 12249-12254, 12257-12265) were designed and synthesized to target 15 of the most highly conserved sites in the 5′UTR of HCV RNA. These synthetic enzymatic nucleic acids were stabilized against nuclease degradation by the addition of modifications such as 2′-O-methyl nucleotides, 2′-amino-uridines at U4 and U7 core positions, phosphorothioate linkages, and a 3′-inverted abasic cap.

In order to mimic cytoplasmic transcription of the HCV genome, OST7 cells were transfected with a target reporter plasmid containing a T7 bacteriophage promoter upstream of a HCV 5′UTR/firefly luciferase fusion gene. Cytoplasmic expression of the target reporter is facilitated by high levels of T7 polymerase expressed in the cytoplasm of OST7 cells. Co-transfection of target reporter HCVT7C₁₋₃₄₁ (firefly luciferase), control reporter pRLSV40 (Renilla luciferase) and enzymatic nucleic acid was carried out in the presence of cationic lipid. To determine the background level of luciferase activity, applicant used a control enzymatic nucleic acid that targets an irrelevant, non-HCV sequence. Transfection of reporter plasmids in the presence of this irrelevant control enzymatic nucleic acid (ICR) resulted in a slight decrease of reporter expression when compared to transfection of reporter plasmids alone. Therefore, the ICR was used to control for non-specific effects on reporter expression during treatment with HCV specific enzymatic nucleic acids. Renilla luciferase expression from the pRLSV40 reporter was used to normalize for transfection efficiency and sample recovery.

Of the 15 amino-modified hammerhead enzymatic nucleic acids tested, 12 significantly inhibited HCV/luciferase expression (>45%, P<0.05) as compared to the ICR (FIG. 9A). These data suggest that most of the HCV 5′UTR sites targeted here are accessible to enzymatic nucleic acid binding and subsequent RNA cleavage. To investigate further the enzymatic nucleic acid-dependent inhibition of HCV/luciferase activity, hammerhead enzymatic nucleic acids designed to cleave after sites 79, 81, 142, 192, 195, 282 or 330 of the HCV 5′UTR were selected for continued study because their anti-HCV activity was the most efficacious over several experiments. A corresponding attenuated core (AC) control was synthesized for each of the 7 active enzymatic nucleic acids (Table VI). Each paired AC control contains similar nucleotide composition to that of its corresponding active enzymatic nucleic acid however, due to scrambled binding arms and changes to the catalytic core, lacks the ability to bind or catalyze the cleavage of HCV RNA. Treatment of OST7 cells with enzymatic nucleic acids designed to cleave after sites 79, 81, 142, 195 or 330 resulted in significant inhibition of HCV/luciferase expression (65%, 50%, 50%, 80% and 80%, respectively) when compared to HCV/luciferase expression in cells treated with corresponding ACs, P<0.05 (FIG. 9B). It should be noted that treatment with either the ICR or ACs for sites 79, 81, 142 or 192 caused a greater reduction of HCV/luciferase expression than treatment with ACs for sites 195, 282 or 330. The observed differences in HCV/luciferase expression after treatment with ACs most likely represents the range of activity due to non-specific effects of oligonucleotide treatment and/or differences in base composition. Regardless of differences in HCV/luciferase expression levels observed as a result of treatment with ACs, active enzymatic nucleic acids designed to cleave after sites 79, 81, 142, 195, or 330 demonstrated similar and potent anti-HCV activity (FIG. 9B).

### Example 7

### Synthetic Stabilized Enzymatic Nucleic Acids Inhibit HCV/Luciferase Expression in a Concentration-Dependent Manner

In order to characterize enzymatic nucleic acid efficacy in greater detail, these same 5 lead hammerhead enzymatic nucleic acids were tested for their ability to inhibit HCV/luciferase expression over a range of enzymatic nucleic acid concentrations (0 nM-100 nM). For constant transfection conditions, the total concentration of nucleic acid was maintained at 100 nM for all samples by mixing the active enzymatic nucleic acid with its corresponding AC. Moreover, mixing of active enzymatic nucleic acid and AC maintains the lipid to nucleic acid charge ratio. A concentration-dependent inhibition of HCV/luciferase expression was observed after treatment with each of the 5 enzymatic nucleic acids (FIGS. 10A-E). By linear interpolation, the enzymatic nucleic acid concentration resulting in 50% inhibition (apparent IC₅₀) of HCV/luciferase expression ranged from 40-215 nM. The two most efficacious enzymatic nucleic acids were those designed to cleave after sites 195 or 330 with apparent IC₅₀ values of 46 nM and 40 nM, respectively (FIGS. 10D and E).

### Example 8

### An Enzymatic Nucleic Acid Mechanism is Required for the Observed Inhibition of HCV/Luciferase Expression

To confirm that an enzymatic nucleic acid mechanism of action was responsible for the observed inhibition of HCV/luciferase expression, paired binding-arm attenuated core (BAC) controls (RPI 15291 and 15294) were synthesized for direct comparison to enzymatic nucleic acids targeting sites 195 (RPI 12252) and 330 (RPI 12254). Paired BACs can specifically bind HCV RNA but are unable to promote RNA cleavage because of changes in the catalytic core and, thus, can be used to assess inhibition due to binding alone. Also included in this comparison were paired SAC controls (RPI 15292 and 15295) that contain scrambled binding arms and attenuated catalytic cores, and so lack the ability to bind the target RNA or to catalyze target RNA cleavage.

Enzymatic nucleic acid cleavage of target RNA should result in both a lower level of HCV/luciferase RNA and a subsequent decrease in HCV/luciferase expression. In order to analyze target RNA levels, a reverse transcriptase/polymerase chain reaction (RT-PCR) assay was employed to quantify HCV/luciferase RNA levels. Primers were designed to amplify the luciferase coding region of the HCV 5′UTR/luciferase RNA. This region was chosen because HCV-targeted enzymatic nucleic acids that might co-purify with cellular RNA would not interfere with RT-PCR amplification of the luciferase RNA region. Primers were also designed to amplify the Renilla luciferase RNA so that Renilla RNA levels could be used to control for transfection efficiency and sample recovery.

OST7 cells were treated with active enzymatic nucleic acids designed to cleave after sites 195 or 330, paired SACs, or paired BACs. Treatment with enzymatic nucleic acids targeting site 195 or 330 resulted in a significant reduction of HCV/luciferase RNA when compared to their paired SAC controls (P<0.01). In this experiment the site 195 enzymatic nucleic acid was more efficacious than the site 330 enzymatic nucleic acid (FIG. 11A). Treatment with paired BACs that target site 195 or 330 did not reduce HCV/luciferase RNA when compared to the corresponding SACs, thus confirming that the ability to bind alone does not result in a reduction of HCV/luciferase RNA.

To confirm that enzymatic nucleic acid-mediated cleavage of target RNA is necessary for inhibition of HCV/luciferase expression, HCV/luciferase activity was determined in the same experiment. As expected, significant inhibition of HCV/luciferase expression was observed after treatment with active enzymatic nucleic acids when compared to paired SACs (FIG. 11B). Importantly, treatment with paired BACs did not inhibit HCV/luciferase expression, thus confirming that the ability to bind alone is also not sufficient to inhibit translation. As observed in the RNA assay, the site 195 enzymatic nucleic acid was more efficacious than the site 330 enzymatic nucleic acid in this experiment. However, a correlation between enzymatic nucleic acid-mediated HCV RNA reduction and inhibition of HCV/luciferase translation was observed for enzymatic nucleic acids to both sites. The reduction in target RNA and the necessity for an active enzymatic nucleic acid catalytic core confirm that a enzymatic nucleic acid mechanism is required for the observed reduction in HCV/luciferase protein activity in cells treated with site 195 or site 330 enzymatic nucleic acids.

### Example 9

### Zinzyme Inhibition of Chimeric HCV/Poliovirus Replication

During HCV infection, viral RNA is present as a potential target for enzymatic nucleic acid cleavage at several processes: un-coating, translation, RNA replication and packaging. Target RNA can be more or less accessible to enzymatic nucleic acid cleavage at any one of these steps. Although the association between the HCV initial ribosome entry site (IRES) and the translation apparatus is mimicked in the HCV 5′UTR/luciferase reporter system, these other viral processes are not represented in the OST7 system. The resulting RNA/protein complexes associated with the target viral RNA are also absent. Moreover, these processes can be coupled in an HCV-infected cell which could further impact target RNA accessibility. Therefore, applicant tested whether enzymatic nucleic acids designed to cleave the HCV 5′UTR could effect a replicating viral system.

Recently, Lu and Wimmer characterized a HCV-poliovirus chimera in which the poliovirus IRES was replaced by the IRES from HCV (Lu & Wimmer, 1996, Proc. Natl. Acad. Sci. USA. 93, 1412-1417). Poliovirus (PV) is a positive strand RNA virus like HCV, but unlike HCV is non-enveloped and replicates efficiently in cell culture. The HCV-PV chimera expresses a stable, small plaque phenotype relative to wild type PV.

The following enzymatic nucleic acid molecules (zinzymes) were synthesized and tested for replicative inhibition of an HCV/Poliovirus chimera: RPI 18763, RPI 18812, RPI 18749, RPI 18765, RPI 18792, and RPI 18814 (Table V). A scrambled attenuated core enzymatic nucleic acid, RPI 18743, was used as a control.

HeLa cells were infected with the HCV-PV chimera for 30 minutes and immediately treated with enzymatic nucleic acid. HeLa cells were seeded in U-bottom 96-well plates at a density of 9000-10,000 cells/well and incubated at 37° C. under 5% CO2 for 24 h. Transfection of nucleic acid (200 nM) was achieved by mixing of 10× nucleic acid (2000 nM) and 10× of a cationic lipid (80 μg/ml) in DMEM (Gibco BRL) with 5% fetal bovine serum (FBS). Nucleic acid/lipid complexes were allowed to incubate for 15 minutes at 37° C. under 5% CO2. Medium was aspirated from cells and replaced with 80 μl of DMEM (Gibco BRL) with 5% FBS serum, followed by the addition of 20 μls of 10× complexes. Cells were incubated with complexes for 24 hours at 37° C. under 5% CO2.

The yield of HCV-PV from treated cells was quantified by plaque assay. The plaque assays were performed by diluting virus samples in serum-free DMEM (Gibco BRL) and applying 100 μl to HeLa cell monolayers (˜80% confluent) in 6-well plates for 30 minutes. Infected monolayers were overlayed with 3 ml 1.2% agar (Sigma) and incubated at 37° C. under 5% CO2. Two or three days later the overlay was removed, monolayers were stained with 1.2% crystal violet, and plaque forming units were counted. The results for the zinzyme inhibition of HCV-PV replication are shown in FIG. 16.

### Example 10

### Antisense Inhibition of Chimeric HCV/Poliovirus Replication

Antisense nucleic acid molecules (RPI 17501 and RPI 17498, Table VII) were tested for replicative inhibition of an HCV/Poliovirus chimera compared to scrambled controls. An antisense nucleic acid molecule is a non-enzymatic nucleic acid molecule that binds to target RNA by means of RNA-RNA or RNA-DNA or RNA-PNA (protein nucleic acid; Egholm et al., 1993 Nature 365, 566) interactions and alters the activity of the target RNA (for a review, see Stein and Cheng, 1993 Science 261, 1004 and Woolf et al., U.S. Pat. No. 5,849,902). Typically, antisense molecules are complementary to a target sequence along a single contiguous sequence of the antisense molecule. However, in certain embodiments, an antisense molecule can bind to substrate such that the substrate molecule forms a loop, and/or an antisense molecule can bind such that the antisense molecule forms a loop. Thus, the antisense molecule can be complementary to two (or even more) non-contiguous substrate sequences or two (or even more) non-contiguous sequence portions of an antisense molecule can be complementary to a target sequence or both. For a review of current antisense strategies, see Schmajuk et al., 1999, J. Biol. Chem., 274, 21783-21789, Delihas et al., 1997, Nature, 15, 751-753, Stein et al., 1997, Antisense N. A. Drug Dev., 7, 151, Crooke, 2000, Methods Enzymol., 313, 3-45; Crooke, 1998, Biotech. Genet. Eng. Rev., 15, 121-157, Crooke, 1997, Ad. Pharmacol., 40, 1-49. In addition, antisense DNA can be used to target RNA by means of DNA-RNA interactions, thereby activating RNase H, which digests the target RNA in the duplex. The antisense oligonucleotides can comprise one or more RNAse H activating region, which is capable of activating RNAse H cleavage of a target RNA. Antisense DNA can be synthesized chemically or expressed via the use of a single stranded DNA expression vector or equivalent thereof. Additionally, antisense molecules can be used in combination with the enzymatic nucleic acid molecules of the instant invention.

A “RNase H activating region” is a region (generally greater than or equal to 4-25 nucleotides in length, preferably from 5-11 nucleotides in length) of a nucleic acid molecule capable of binding to a target RNA to form a non-covalent complex that is recognized by cellular RNase H enzyme (see for example Arrow et al., U.S. Pat. No. 5,849,902; Arrow et al., U.S. Pat. No. 5,989,912). The RNase H enzyme binds to the nucleic acid molecule-target RNA complex and cleaves the target RNA sequence. The RNase H activating region comprises, for example, phosphodiester, phosphorothioate (preferably at least four of the nucleotides are phosphorothiote substitutions; more specifically, 4-11 of the nucleotides are phosphorothiote substitutions); phosphorodithioate, 5′-thiophosphate, or methylphosphonate backbone chemistry or a combination thereof. In addition to one or more backbone chemistries described above, the RNase H activating region can also comprise a variety of sugar chemistries. For example, the RNase H activating region can comprise deoxyribose, arabino, fluoroarabino or a combination thereof, nucleotide sugar chemistry. Those skilled in the art will recognize that the foregoing are non-limiting examples and that any combination of phosphate, sugar and base chemistry of a nucleic acid that supports the activity of RNase H enzyme is within the scope of the definition of the RNase H activating region and the instant invention.

HeLa cells were infected with the HCV-PV chimera for 30 minutes and immediately treated with antisense nucleic acid. HeLa cells were seeded in U-bottom 96-well plates at a density of 9000-10,000 cells/well and incubated at 37° C. under 5% CO2 for 24 h. Transfection of nucleic acid (200 nM) was achieved by mixing of 10× nucleic acid (2000 nM) and 10× of a cationic lipid (80 μg/ml) in DMEM (Gibco BRL) with 5% fetal bovine serum (FBS). Nucleic acid/lipid complexes were allowed to incubate for 15 minutes at 37° C. under 5% CO2. Medium was aspirated from cells and replaced with 80 μl of DMEM (Gibco BRL) with 5% FBS serum, followed by the addition of 20 μls of 10× complexes. Cells were incubated with complexes for 24 hours at 37° C. under 5% CO2.

The yield of HCV-PV from treated cells was quantified by plaque assay. The plaque assays were performed by diluting virus samples in serum-free DMEM (Gibco BRL) and applying 100 μl to HeLa cell monolayers (˜80% confluent) in 6-well plates for 30 minutes. Infected monolayers were overlayed with 3 ml 1.2% agar (Sigma) and incubated at 37° C. under 5% CO2. Two or three days later the overlay was removed, monolayers were stained with 1.2% crystal violet, and plaque forming units were counted. The results for the antisense inhibition of HCV-PV are shown in FIG. 17.

### Example 11

### Nucleic Acid Inhibition of Chimeric HCV/PV in Combination with Interferon

One of the limiting factors in interferon (IFN) therapy for chronic HCV are the toxic side effects associated with IFN. Applicant has reasoned that lowering the dose of IFN needed can reduce these side effects. Applicant has previously shown that enzymatic nucleic acid molecules targeting HCV RNA have a potent antiviral effect against replication of an HCV-poliovirus (PV) chimera (Macejak et al., 2000, Hepatology, 31, 769-776). In order to determine if the antiviral effect of type 1 IFN could be improved by the addition of anti-HCV enzymatic nucleic acid treatment, a dose response (0 U/ml to 100 U/ml) with IFN alfa 2a or IFN alfa 2b was performed in HeLa cells in combination with 200 nM site 195 anti-HCV enzymatic nucleic acid (RPI 13919) or enzymatic nucleic acid control (SAC) treatment. The SAC control (RPI 17894) is a scrambled binding arm, attenuated core version of the site 195 enzymatic nucleic acid (RPI 13919). IFN dose responses were performed with different pretreatment regimes to find the dynamic range of inhibition in this system. In these studies, HeLa cells were used instead of HepG2 because of more efficient enzymatic nucleic acid delivery (Macejak et al., 2000, Hepatology, 31, 769-776).

Cells and Virus

HeLa cells were maintained in DMEM (BioWhittaker, Walkersville, Md.) supplemented with 5% fetal bovine serum. A cloned DNA copy of the HCV-PV chimeric virus was a gift of Dr. Eckard Wimmer (NYU, Stony Brook, N.Y.). An RNA version was generated by in vitro transcription and transfected into HeLa cells to produce infectious virus (Lu and Wimmer, 1996, PNAS USA., 93, 1412-1417).

Enzymatic Nucleic Acid Synthesis

Nuclease resistant enzymatic nucleic acids and control oligonucleotides containing 2′-O-methyl-nucleotides, 2′-deoxy-2′-C-allyl uridine, a 3′-inverted abasic cap, and phosphorothioate linkages were chemically synthesized. The anti-HCV enzymatic nucleic acid (RPI 13919) targeting cleavage after nucleotide 195 of the 5′ UTR of HCV is shown in Table V. Attenuated core controls have nucleotide changes in the core sequence that greatly diminished the enzymatic nucleic acid's cleavage activity. The attenuated controls either contain scrambled binding arms (referred to as SAC, RPI 18743) or maintain binding arms (BAC, RPI 17894) capable of binding to the HCV RNA target.

Enzymatic Nucleic Acid Delivery

A cationic lipid was used as a cytofectin agent. HeLa cells were seeded in 96-well plates at a density of 9000-10,000 cells/well and incubated at 37° C. under 5% CO2 for 24 h. Transfection of enzymatic nucleic acid or control oligonucleotides (200 nM) was achieved by mixing 10× enzymatic nucleic acid or control oligonucleotides (2000 nM) with 10× RPI.9778 (80 μg/ml) in DMEM containing 5% fetal bovine serum (FBS) in U-bottom 96-well plates to make 5× complexes. Enzymatic nucleic acid/lipid complexes were allowed to incubate for 15 min at 37° C. under 5% CO2. Medium was aspirated from cells and replaced with 80 μl of DMEM (Gibco BRL) containing 5% FBS serum, followed by the addition of 20 μl of 5× complexes. Cells were incubated with complexes for 24 h at 37° C. under 5% CO2.

Interferon/Enzymatic Nucleic Acid Combination Treatment

Interferon alfa 2a (Roferon®) was purchased from Roche Bioscience (Palo Alto, Calif.). Interferon alfa 2b (Intron A®) was purchased from Schering-Plough Corporation (Madison, N.J.). Consensus interferon (interferon-alfa-con 1) was a generous gift of Amgen, Inc. (Thousand Oaks, Calif.). For the basis of comparison, the manufacturers' specified units were used in the studies reported here; however, the manufacturers' unit definitions of these three IFN preparations are not necessarily the same. Nevertheless, since clinical dosing is based on the manufacturers' specified units, a direct comparison based on these units has relevance to clinical therapeutic indices. HeLa cells were seeded (10,000 cells per well) and incubated at 37° C. under 5% CO2 for 24 h. Cells were then pre-treated with interferon in complete media (DMEM+5% FBS) for 4 h and then infected with HCV-PV at a multiplicity of infection (MOI)=0.1 for 30 min. The viral inoculum was then removed and enzymatic nucleic acid or attenuated control (SAC or BAC) was delivered with the cytofectin formulation (8 μg/ml) in complete media for 24 h as described above. Where indicated for enzymatic nucleic acid dose response studies, active enzymatic nucleic acid was mixed with SAC to maintain a 200 nM total oligonucleotide concentration and the same lipid charge ratio. After 24 h, cells were lysed to release virus by three cycles of freeze/thaw. Virus was quantified by plaque assay and viral yield is reported as mean plaque forming units per ml (pfu/ml)+SD. All experiments were repeated at least twice and the trends in the results reported were reproducible. Significance levels (P values) were determined by the Student's test.

Plaque Assay

Virus samples were diluted in serum-free DMEM and 100 μl applied to Vero cell monolayers (˜80% confluent) in 6-well plates for 30 min. Infected monolayers were overlaid with 3 ml 1.2% agar (Sigma Chemical Company, St. Louis, Mo.) and incubated at 37° C. under 5% CO2. When plaques were visible (after two to three days) the overlay was removed, monolayers were stained with 1.2% crystal violet, and plaque forming units were counted.

Results

As shown in FIGS. 12A and 12B, treatment with the site 195 (RPI 13919) anti-HCV hammerhead enzymatic nucleic acid alone (0 U/ml IFN) resulted in viral replication that was dramatically reduced compared to SAC-treated cells (85%, P<0.01). For both IFN alfa 2a (FIG. 12A) or IFN alfa 2b (FIG. 12B), treatment with 25 U/ml resulted in a ˜90% inhibition of HCV-PV replication in SAC-treated cells as compared to cells treated with SAC alone (p<0.0l for both observations). The maximal level of inhibition in SAC-treated cells (94%) was achieved by treatment with ≧50U/ml of either IFN alfa 2a or IFN alfa 2b (p<0.01 for both observations versus SAC alone). Maximal inhibition could however, be achieved by a 5-fold lower dose of IFN alfa 2a (10 U/ml) if enzymatic nucleic acid targeting site 195 in the 5′ UTR of HCV RNA was given in combination (FIG. 12A, p<0.01). While the additional effect of enzymatic nucleic acid treatment on IFN alfa 2b-treated cells at 10 U/ml was very slight, the combined effect with 25 U/ml IFN alfa 2b was greater in magnitude (FIG. 12B). For both interferons tested, pretreatment with 25 U/ml in combination with 200 nM site 195 anti-HCV enzymatic nucleic acid resulted in an even greater level of inhibition of viral replication (>98%) compared to replication in cells treated with 200 nM SAC alone (P<0.01).

A dose response of the site 195 anti-HCV enzymatic nucleic acid was also performed in HeLa cells, either with or without 12.5 U/ml IFN alfa 2a or IFN alfa 2b pretreatment. As shown in FIG. 13, enzymatic nucleic acid-mediated inhibition was dose-dependent and a significant inhibition of HCV-PV replication (>75% versus 0 nM enzymatic nucleic acid, P<0.01) could be achieved by treatment with ≧150 nM anti-HCV enzymatic nucleic acid alone (no IFN). However, in IFN-pretreated cells, the dose of anti-HCV enzymatic nucleic acid needed to achieve this level of inhibition was decreased 3-fold to 50 nM (P<0.01 versus 0 nM enzymatic nucleic acid). In comparison, treatment with the site 195 anti-HCV enzymatic nucleic acid alone at 50 nM resulted in only ˜40% inhibition of virus replication. Pretreatment with IFN enhanced the antiviral effect of site 195 enzymatic nucleic acid at all enzymatic nucleic acid doses, compared to no IFN pretreatment.

Interferon-alfacon1, consensus IFN (CIFN), is another type 1 IFN that is used to treat chronic HCV. To determine if a similar enhancement can occur in CIFN-treated cells, a dose response with CIFN was performed in HeLa cells using 0 U/ml to 12.5 U/ml CIFN in combination with 200 nM site 195 anti-HCV enzymatic nucleic acid or SAC treatment (FIG. 14A). Again, in the presence of the site 195 anti-HCV enzymatic nucleic acid alone, viral replication was dramatically reduced compared to SAC-treated cells. As shown in FIG. 14A, treatment with 200 nM anti-HCV enzymatic nucleic acid alone significantly inhibited HCV-PV replication (90% versus SAC treatment, P<0.01). However, pretreatment with concentrations of CIFN from 1 U/ml to 12.5 U/ml in combination with 200 nM anti-HCV enzymatic nucleic acid resulted in even greater inhibition of viral replication (>98%) compared to replication in cells treated with 200 nM SAC alone (P<0.01). It is important to note that pretreatment with 1 U/ml CIFN in SAC-treated cells did not have a significant effect on HCV-poliovirus replication, but in the presence of enzymatic nucleic acid a significant inhibition of replication was observed (>98%, P<0.01). Thus, the dose of CIFN needed to achieve a >98% inhibition could be lowered to 1 U/ml in cells also treated with 200 nM site 195 anti-HCV enzymatic nucleic acid.

A dose response of site 195 anti-HCV enzymatic nucleic acid was then performed in HeLa cells, either with or without 12.5 U/ml CIFN pretreatment. As shown in FIG. 14B, a significant inhibition of HCV-PV replication (>95% versus 0 nM enzymatic nucleic acid, P<0.01) could be achieved by treatment with ≧150 nM anti-HCV enzymatic nucleic acid alone. However, in CIFN-pretreated cells, the dose of anti-HCV enzymatic nucleic acid needed to achieve this level of inhibition was only 50 nM (P<0.01). In comparison, treatment with the site 195 anti-HCV enzymatic nucleic acid alone at 50 nM resulted in ˜50% inhibition of virus replication. Thus, as was seen with IFN alfa 2a and IFN alfa 2b, the dose of enzymatic nucleic acid could be reduced 3-fold in the presence of CIFN pretreatment to achieve a similar antiviral effect as enzymatic nucleic acid-treatment alone.

To further explore the combination of lower enzymatic nucleic acid concentration and CIFN, a dose response with 0 U/ml to 12.5 U/ml CIFN was subsequently performed in HeLa cells in combination with 50 nM site 195 anti-HCV enzymatic nucleic acid treatment. In multiple experiments, treatment with 50 nM anti-HCV enzymatic nucleic acid alone inhibited HCV-PV replication 50%-81% compared to viral replication in SAC-treated cells. As for the experiment shown in FIG. 14A, treatment with CIFN alone at 5 U/ml resulted in ˜50% inhibition of viral replication. However, a four hour pretreatment with 5 U/ml CIFN followed by 50 nM anti-HCV enzymatic nucleic acid treatment resulted in 95%-97% inhibition compared to SAC-treated cells (P<0.01).

To demonstrate that the enhanced antiviral effect of CIFN and enzymatic nucleic acid combination treatment was dependent upon enzymatic nucleic acid cleavage activity, the effect of CIFN in combination with site 195 anti-HCV enzymatic nucleic acid versus the effect of CIFN in combination with a binding competent, attenuated core, control (BAC) was then compared. The BAC can still bind to its specific RNA target, but is greatly diminished in cleavage activity. Pretreatment with 12.5 U/ml CIFN reduced the viral yield ˜90% (7-fold) in cells treated with BAC (compare CIFN versus BAC in FIG. 15). Cells treated with 200 nM site 195 anti-HCV enzymatic nucleic acid alone produced 95% (17-fold) less virus than BAC-treated cells (195 RZ BAC in FIG. 15). The combination of CIFN pretreatment and 200 nM site 195 anti-HCV enzymatic nucleic acid results in an augmented >98% (300-fold) reduction in viral yield (CIFN+RZ versus control in FIG. 15).

2′-5′-Oligoadenylate Inhibition of HCV

Type 1 Interferon is a key constituent of many effective treatment programs for chronic HCV infection. Treatment with type 1 interferon induces a number of genes and results in an antiviral state within the cell. One of the genes induced is 2′, 5′ oligoadenylate synthetase, an enzyme that synthesizes short 2′, 5′ oligoadenylate (2-5A) molecules. Nascent 2-5A subsequently activates a latent RNase, RNase L, which in turn nonspecifically degrades viral RNA. As described herein, ribozymes targeting HCV RNA that inhibit the replication of an HCV-poliovirus (HCV-PV) chimera in cell culture and have shown that this antiviral effect is augmented if ribozyme is given in combination with type 1 interferon. In addtion, the 2-5A component of the interferon response can also inhibit replication of the HCV-PV chimera.

The antiviral effect of anti-HCV ribozyme treatment is enhanced if type 1 interferon is given in combination. Interferon induces a number of gene products including 2′,5′ oligoadenylate (2-5A) synthetase, double-stranded RNA-activated protein kinase (PKR), and the Mx proteins. Mx proteins appear to interfere with nuclear transport of viral complexes and are not thought to play an inhibitory role in HCV infection. On the other hand, the additional 2-5A-mediated RNA degradation (via RNase L) and/or the inhibition of viral translation by PKR in interferon-treated cells can augment the ribozyme-mediated inhibition of HCV-PV replication.

To investigate the potential role of the 2-5A/RNase L pathway in this enhancement phenomenon, HCV-PV replication was analyzed in HeLa cells treated exogenously with chemically-synthesized analogs of 2-5A (FIG. 18), alone and in combination with the anti-HCV ribozyme (RPI 13919). These results were compared to replication in cells treated with interferon and/or anti-HCV ribozyme. Anti-HCV ribozyme was transfected into cells with a cationic lipid. To control for nonspecific effects due to lipid-mediated transfection, a scrambled arm, attenuated core, oligonucleotide (SAC) (RPI 17894) was transfected for comparison. The SAC is the same base composition as the ribozyme but is greatly attenuated in catalytic activity due to changes in the core sequence and cannot bind specifically to the HCV sequence.

As shown in FIG. 19A, HeLa cells pretreated with 10 U/ml consensus interferon for 4 hours prior to HCV-PV infection resulted in ˜70% reduction of viral replication in SAC-treated cells. Similarly, HeLa cells treated with 100 nM anti-HCV ribozyme for 20 hours after infection resulted in an ˜80% reduction in viral yield. This antiviral effect was enhanced to ˜98% inhibition in HeLa cells pretreated with interferon for 4 hours before infection and then treated with anti-HCV ribozyme for 20 hours after infection. In parallel, a 2-5A compound (analog I, FIG. 18) that was protected from nuclease digestion at the 3′-end with an inverted abasic moiety was tested. As shown in FIG. 19B, treatment with 200 nM 2-5A analog I for 4 hours prior to HCV-PV infection only slightly inhibited HCV-PV replication (˜20%) in SAC-treated cells. Moreover, the inhibition due to a 20 hour anti-HCV ribozyme treatment was not augmented with a 4 hour pretreatment of 2-5A in combination (compare third bar to fourth bar in FIG. 19B).

There are several possible possible explanations why the chemically synthesized 2-5A analog was not able to completely activate RNase L. It is possible that the 2-5A analog was not sufficiently stable or that in this experiment the 4 hour pretreatment period was too short for RNase L activation. To test these possibilities, a 2-5A compound containing a 5′-terminal thiophosphate (P═S) for added nuclease resistance, in addition to the 3′-abasic, was also included (analog II, FIG. 18). In addition, a longer 2-5A treatment was used. In this experiment (FIG. 20), HeLa cells were treated with 2-5A or 2-5A(P═S) for 20 hours after HCV-PV infection. Again, anti-HCV ribozyme treatment resulted in >80% inhibition. In contrast to the 20% inhibition of viral replication seen with a 4 hour 2-5A pretreatment, viral replication in cells treated with 2-5A analog I for 20 hours after HCV-PV infection was inhibited by ˜70%. The P═S version (analog II) inhibited HCV-PV replication by 35%. Thus, both 2-5A analogs used here are able to generate an antiviral effect, presumably through RNase L activation. The P═S version, although more resistant to 5′ dephosphorylation, did not yield as great an anti-viral effect. It is possible that combination of the 5′-terminal thiophosphate together with the presence of a 3′-inverted abasic moiety can interfere with RNase L activation. Nevertheless, these results demonstrate potent anti-HCV activity by a nuclease-stabilized 2-5A analog.

The level of reduction in HCV-PV replication in cells treated with 2-5A analog I for 20 hours was similar to that in cells pretreated with consensus interferon for 4 hours. To determine if this expanded 2-5A treatment regimen would enhance anti-HCV ribozyme efficacy to the same degree as does the interferon pretreatment, HeLa cells infected with HCV-PV were treated with a combination of 2-5A and anti-HCV ribozyme for 20 hours after infection. In this experiment, a 200 nM treatment with anti-HCV ribozyme or 2-5A treatment alone inhibited viral replication by 88% or ˜60%, respectively, compared to SAC treatment (FIG. 21, left three bars). To maintain consistent transfection conditions but vary the concentration of anti-HCV ribozyme or 2-5A, anti-HCV ribozyme was mixed with the SAC to maintain a total dose of 200 nM. A 50 nM treatment with anti-HCV ribozyme inhibited HCV-PV replication by ˜70% (solid middle bar). However, the amount of HCV-PV replication was not further reduced in cells treated with a combination of 50 nM anti-HCV ribozyme and 150 nM 2-5A (striped middle bar). Likewise, cells treated with 100 nM anti-HCV ribozyme inhibited HCV-PV replication by ˜80% whether they were also treated with 100 nM of 2-5A or SAC (right two bars). In contrast, antiviral activity increased from 80% to 98% when 100 nM anti-HCV ribozyme was given in combination with interferon (FIG. 19A). The reasons for the lack of additive or synergistic effects for the ribozyme/2-5A combination therapy is unclear at this time but can be due to that fact that both compounds have a similar mechanism of action (degradation of RNA). Further study is warranted to examine this possibility.

As a monotherapy, 2-5A treatment generates a similar inhibitory effect on HCV-poliovirus replication as does interferon treatment. If these results are maintained in HCV patients, treatment with 2-5A can not only be efficacious but can also generate less side effects than those observed with interferon if the plethora of interferon-induced genes were not activated.

Cell Culture Assays Although there have been reports of replication of HCV in cell culture (see below), these systems are difficult to replicate and have proven unreliable. Therefore, as was the case for development of other anti-HCV therapeutics such as interferon and ribavirin, after demonstration of safety in animal studies applicant can proceed directly into a clinical feasibility study.

Several recent reports have documented in vitro growth of HCV in human cell lines (Mizutani et al., Biochem Biophys Res Commun 1996 227(3):822-826; Tagawa et al., Journal of Gasteroenterology and Hepatology 1995 10(5):523-527; Cribier et al., Journal of General Virology 76(10):2485-2491; Seipp et al., Journal of General Virology 1997 78(10)2467-2478; Iacovacci et al., Research Virology 1997 148(2):147-151; Iocavacci et al., Hepatology 1997 26(5) 1328-1337; Ito et al., Journal of General Virology 1996 77(5):1043-1054; Nakajima et al., Journal of Virology 1996 70(5):3325-3329; Mizutani et al., Journal of Virology 1996 70(10):7219-7223; Valli et al., Res Virol 1995 146(4): 285-288; Kato et al., Biochem Biophys Res Comm 1995 206(3):863-869). Replication of HCV has been demonstrated in both T and B cell lines as well as cell lines derived from human hepatocytes. Demonstration of replication was documented using either RT-PCR based assays or the b-DNA assay. It is important to note that the most recent publications regarding HCV cell cultures document replication for up to 6-months.

Additionally, another recent study has identified more robust strains of hepatitis C virus having adaptive mutations that allow the strains to replicate more vigorously in human cell culture, Blight et al., Science, 290: 1972-1974 (2000). The mutations that confer this enhanced ability to replicate are located in a specific region of a protein identified as NS5A. These studies showed that in certain cell culture systems, infection with the robust strains produces a 10,000-fold increase in the number of infected cells. The greatly increased availability of HCV-infected cells in culture can be used to develop high-throughput screening assays, in which a large number of compounds, such as enzymatic nucleic acid molecules, can be tested to determine their effectiveness.

In addition to cell lines that can be infected with HCV, several groups have reported the successful transformation of cell lines with cDNA clones of full-length or partial HCV genomes (Harada et al., Journal of General Virology 1995 76(5)1215-1221; Haramatsu et al., Journal of Viral Hepatitis 1997 4S(1):61-67; Dash et al., American Journal of Pathology 1997 151(2):363-373; Mizuno et al., Gasteroenterology 1995 109(6):1933-40; Yoo et al., Journal Of Virology 1995 69(1):32-38).

Animal Models

The best characterized animal system for HCV infection is the chimpanzee. Moreover, the chronic hepatitis that results from HCV infection in chimpanzees and humans is very similar. Although clinically relevant, the chimpanzee model suffers from several practical impediments that make use of this model difficult. These include; high cost, long incubation requirements and lack of sufficient quantities of animals. Due to these factors, a number of groups have attempted to develop rodent models of chronic hepatitis C infection. While direct infection has not been possible several groups have reported on the stable transfection of either portions or entire HCV genomes into rodents (Yamamoto et al., Hepatology 1995 22(3): 847-855; Galun et al., Journal of Infectious Disease 1995 172(1):25-30; Koike et al., Journal of general Virology 1995 76(12)3031-3038; Pasquinelli et al., Hepatology 1997 25(3): 719-727; Hayashi et al, Princess Takamatsu Symp 1995 25:1430149; Mariya K, Yotsuyanagi H, Shintani Y, Fujie H, Ishibashi K, Matsuura Y, Miyamura T, Koike K. Hepatitis C virus core protein induces hepatic steatosis in transgenic mice. Journal of General Virology 1997 78(7) 1527-1531; Takehara et al., Hepatology 1995 21(3):746-751; Kawamura et al., Hepatology 1997 25(4): 1014-1021). In addition, transplantation of HCV infected human liver into immunocompromised mice results in prolonged detection of HCV RNA in the animal's blood.

Vierling, International PCT Publication No. WO 99/16307, describes a method for expressing hepatitis C virus in an in vivo animal model. Viable, HCV infected human hepatocytes are transplanted into a liver parenchyma of a scid/scid mouse host. The scid/scid mouse host is then maintained in a viable state, whereby viable, morphologically intact human hepatocytes persist in the donor tissue and hepatitis C virus is replicated in the persisting human hepatocytes. This model provides an effective means for the study of HCV inhibition by enzymatic nucleic acids in vivo.

Diagnostic Uses

Enzymatic nucleic acids of this invention can be used as diagnostic tools to examine genetic drift and mutations within diseased cells or to detect the presence of HCV RNA in a cell. The close relationship between enzymatic nucleic acid activity and the structure of the target RNA allows the detection of mutations in any region of the molecule, which alters the base-pairing and three-dimensional structure of the target RNA. By using multiple enzymatic nucleic acids described in this invention, one can map nucleotide changes, which are important to RNA structure and function in vitro, as well as in cells and tissues. Cleavage of target RNAs with enzymatic nucleic acids can be used to inhibit gene expression and define the role (essentially) of specified gene products in the progression of disease. In this manner, other genetic targets can be defined as important mediators of the disease. These experiments will lead to better treatment of the disease progression by affording the possibility of combination therapies (e.g., multiple enzymatic nucleic acids targeted to different genes, enzymatic nucleic acids coupled with known small molecule inhibitors, or intermittent treatment with combinations of enzymatic nucleic acids and/or other chemical or biological molecules). Other in vitro uses of enzymatic nucleic acids of this invention are well known in the art, and include detection of the presence of mRNAs associated with HCV related condition. Such RNA is detected by determining the presence of a cleavage product after treatment with a enzymatic nucleic acid using standard methodology.

In a specific example, enzymatic nucleic acids which cleave only wild-type or mutant forms of the target RNA are used for the assay. The first enzymatic nucleic acid is used to identify wild-type RNA present in the sample and the second enzymatic nucleic acid is used to identify mutant RNA in the sample. As reaction controls, synthetic substrates of both wild-type and mutant RNA are cleaved by both enzymatic nucleic acids to demonstrate the relative enzymatic nucleic acid efficiencies in the reactions and the absence of cleavage of the “non-targeted” RNA species. The cleavage products from the synthetic substrates also serve to generate size markers for the analysis of wild-type and mutant RNAs in the sample population. Thus each analysis requires two enzymatic nucleic acids, two substrates and one unknown sample which are combined into six reactions. The presence of cleavage products is determined using an RNase protection assay so that full-length and cleavage fragments of each RNA can be analyzed in one lane of a polyacrylamide gel. It is not absolutely required to quantify the results to gain insight into the expression of mutant RNAs and putative risk of the desired phenotypic changes in target cells. The expression of mRNA whose protein product is implicated in the development of the phenotype (i.e., HCV) is adequate to establish risk. If probes of comparable specific activity are used for both transcripts, then a qualitative comparison of RNA levels is adequate and will decrease the cost of the initial diagnosis. Higher mutant form to wild-type ratios are correlated with higher risk whether RNA levels are compared qualitatively or quantitatively.

Additional Uses

Potential usefulness of sequence-specific enzymatic nucleic acid molecules of the instant invention have many of the same applications for the study of RNA that DNA restriction endonucleases have for the study of DNA (Nathans et al., 1975 Ann. Rev. Biochem. 44:273). For example, the pattern of restriction fragments can be used to establish sequence relationships between two related RNAs, and large RNAs can be specifically cleaved to fragments of a size more useful for study. The ability to engineer sequence specificity of the enzymatic nucleic acid molecule is ideal for cleavage of RNAs of unknown sequence. Applicant describes the use of nucleic acid molecules to down-regulate gene expression of target genes in bacterial, microbial, fungal, viral, and eukaryotic systems including plant, or mammalian cells.

### TABLE 1

Characteristics of Naturally Occurring Ribozymes

Group I Introns

Size: ˜150 to >1000 nucleotides.

Requires a U in the target sequence immediately 5′ of the cleavage site.

Binds 4-6 nucleotides at the 5′-side of the cleavage site.

Reaction mechanism: attack by the 3′-OH of guanosine to generate cleavage products with 3′-OH and 5′-guanosine.

Additional protein cofactors required in some cases to help folding and maintenance of the active structure. [1]

Over 300 known members of this class. Found as an intervening sequence in Tetrahymena thermophila rRNA, fungal mitochondria, chloroplasts, phage T4, blue-green algae, and others.

Major structural features largely established through phylogenetic comparisons, mutagenesis, and biochemical studies [1,2].

Complete kinetic framework established for one ribozyme [3,4,5,6].

Studies of ribozyme folding and substrate docking underway [7,8,9].

Chemical modification investigation of important residues well established [10,11].

The small (4-6 nt) binding site may make this ribozyme too non-specific for targeted RNA cleavage, however, the Tetrahymena group I intron has been used to repair a “defective” β-galactosidase message by the ligation of new β-galactosidase sequences onto the defective message [12].

RNAse P RNA (M1 RNA)

Size: ˜290 to 400 nucleotides.

RNA portion of a ubiquitous ribonucleoprotein enzyme.

Cleaves tRNA precursors to form mature tRNA [13].

Reaction mechanism: possible attack by M²⁺-OH to generate cleavage products with 3′-OH and 5′-phosphate.

RNAse P is found throughout the prokaryotes and eukaryotes. The RNA subunit has been sequenced from bacteria, yeast, rodents, and primates.

Recruitment of endogenous RNAse P for therapeutic applications is possible through hybridization of an External Guide Sequence (EGS) to the target RNA [14,15]

Important phosphate and 2′ OH contacts recently identified [16,17]

Group II Introns

Size: >1000 nucleotides.

Trans cleavage of target RNAs recently demonstrated [18,19].

Sequence requirements not fully determined.

Reaction mechanism: 2′-OH of an internal adenosine generates cleavage products with 3′-OH and a “lariat” RNA containing a 3′-5′ and a 2′-5′ branch point.

Only natural ribozyme with demonstrated participation in DNA cleavage [20,21] in addition to RNA cleavage and ligation.

Major structural features largely established through phylogenetic comparisons [22].

Important 2′ OH contacts beginning to be identified [23]

Kinetic framework under development [24]

Neurospora VS RNA

Size: ˜144 nucleotides.

Trans cleavage of hairpin target RNAs recently demonstrated [25].

Sequence requirements not fully determined.

Reaction mechanism: attack by 2′-OH 5′ to the scissile bond to generate cleavage products with 2′,3′-cyclic phosphate and 5′-OH ends.

Binding sites and structural requirements not fully determined.

Only 1 known member of this class. Found in Neurospora VS RNA.

Hammerhead Ribozyme

(see text for references)

Size: ˜13 to 40 nucleotides.

Requires the target sequence UH immediately 5′ of the cleavage site.

Binds a variable number nucleotides on both sides of the cleavage site.

Reaction mechanism: attack by 2′-OH 5′ to the scissile bond to generate cleavage products with 2′,3′-cyclic phosphate and 5′-OH ends.

14 known members of this class. Found in a number of plant pathogens (virusoids) that use RNA as the infectious agent.

Essential structural features largely defined, including 2 crystal structures [26, 27]

Minimal ligation activity demonstrated (for engineering through in vitro selection) [28]

Complete kinetic framework established for two or more ribozymes [29]. Chemical modification investigation of important residues well established [30].

Hairpin Ribozyme

Size: ˜50 nucleotides.

Requires the target sequence GUC immediately 3! of the cleavage site.

Binds 4-6 nucleotides at the 5′-side of the cleavage site and a variable number to the 3′-side of the cleavage site.

Reaction mechanism: attack by 2′-OH 5′ to the scissile bond to generate cleavage products with 2′,3′-cyclic phosphate and 5′-OH ends.

3 known members of this class. Found in three plant pathogen (satellite RNAs of the tobacco ringspot virus, arabis mosaic virus and chicory yellow mottle virus) which uses RNA as the infectious agent.

Essential structural features largely defined [31, 32, 33, 34]

Ligation activity (in addition to cleavage activity) makes ribozyme amenable to engineering through in vitro selection [35]

Complete kinetic framework established for one ribozyme [36].

Chemical modification investigation of important residues begun [37, 38].

Hepatitis Delta Virus (HDV) Ribozyme

Size: ˜60 nucleotides.

Trans cleavage of target RNAs demonstrated [39].

Binding sites and structural requirements not fully determined, although no sequences 5′ of cleavage site are required. Folded ribozyme contains a pseudoknot structure [40].

Reaction mechanism: attack by 2′-OH 5′ to the scissile bond to generate cleavage products with 2′,3′-cyclic phosphate and 5′-OH ends.

Only 2 known members of this class. Found in human HDV.

Circular form of HDV is active and shows increased nuclease stability [41]

1. Michel, Francois; Westhof, Eric. Slippery substrates. Nat. Struct. Biol. (1994), 1(1), 5-7.

2. Lisacek, Frederique; Diaz, Yolande; Michel, Francois. Automatic identification of group I intron cores in genomic DNA sequences. J. Mol. Biol. (1994), 235(4), 1206-17.

3. Herschlag, Daniel; Cech, Thomas R. Catalysis of RNA cleavage by the Tetrahymena thermophila ribozyme. 1. Kinetic description of the reaction of an RNA substrate complementary to the active site. Biochemistry (1990), 29(44), 10159-71.

4. Herschlag, Daniel; Cech, Thomas R. Catalysis of RNA cleavage by the Tetrahymena thermophila ribozyme. 2. Kinetic description of the reaction of an RNA substrate that forms a mismatch at the active site. Biochemistry (1990), 29(44), 10172-80.

5. Knitt, Deborah S.; Herschlag, Daniel. pH Dependencies of the Tetrahymena Ribozyme Reveal an Unconventional Origin of an Apparent pKa. Biochemistry (1996), 35(5), 1560-70.

6. Bevilacqua, Philip C.; Sugimoto, Naoki; Turner, Douglas H. A mechanistic framework for the second step of splicing catalyzed by the Tetrahymena ribozyme. Biochemistry (1996), 35(2), 648-58.

7. Li, Yi; Bevilacqua, Philip C.; Mathews, David; Turner, Douglas H. Thermodynamic and activation parameters for binding of a pyrene-labeled substrate by the Tetrahymena ribozyme: docking is not diffusion-controlled and is driven by a favorable entropy change. Biochemistry (1995), 34(44), 14394-9.

8. Banerjee, Aloke Raj; Turner, Douglas H. The time dependence of chemical modification reveals slow steps in the folding of a group I ribozyme. Biochemistry (1995), 34(19), 6504-12.

9. Zarrinkar, Patrick P.; Williamson, James R. The P9.1-P9.2 peripheral extension helps guide folding of the Tetrahymena ribozyme. Nucleic Acids Res. (1996), 24(5), 854-8.

10. Strobel, Scott A.; Cech, Thomas R. Minor groove recognition of the conserved G.cntdot.U pair at the Tetrahymena ribozyme reaction site. Science (Washington, D. C.) (1995), 267(5198), 675-9.

11. Strobel, Scott A.; Cech, Thomas R. Exocyclic Amine of the Conserved G.cntdot.U Pair at the Cleavage Site of the Tetrahymena Ribozyme Contributes to 5′-Splice Site Selection and Transition State Stabilization. Biochemistry (1996), 35(4), 1201-11.

12. Sullenger, Bruce A.; Cech, Thomas R. Ribozyme-mediated repair of defective mRNA by targeted trans-splicing. Nature (London) (1994), 371(6498), 619-22.

13. Robertson, H. D.; Altman, S.; Smith, J. D. J. Biol. Chem., 247, 5243-5251 (1972).

14. Forster, Anthony C.; Altman, Sidney. External guide sequences for an RNA enzyme. Science (Washington, D.C., 1883-) (1990), 249(4970), 783-6.

15. Yuan, Y.; Hwang, E. S.; Altman, S. Targeted cleavage of mRNA by human RNase P. Proc. Natl. Acad. Sci. USA (1992) 89, 8006-10.

16. Harris, Michael E.; Pace, Norman R. Identification of phosphates involved in catalysis by the ribozyme RNase P RNA. RNA (1995), 1(2), 210-18.

17. Pan, Tao; Loria, Andrew; Zhong, Kun. Probing of tertiary interactions in RNA: 2′-hydroxyl-base contacts between the RNase P RNA and pre-tRNA. Proc. Natl. Acad. Sci. U.S. A. (1995), 92(26), 12510-14.

18. Pyle, Anna Marie; Green, Justin B. Building a Kinetic Framework for Group II Intron Ribozyme Activity: Quantitation of Interdomain Binding and Reaction Rate. Biochemistry (1994), 33(9), 2716-25.

19. Michels, William J. Jr.; Pyle, Anna Marie. Conversion of a Group II Intron into a New Multiple-Turnover Ribozyme that Selectively Cleaves Oligonucleotides: Elucidation of Reaction Mechanism and Structure/Function Relationships. Biochemistry (1995), 34(9), 2965-77.

20. Zimmerly, Steven; Guo, Huatao; Eskes, Robert; Yang, Jian; Perlman, Philip S.; Lambowitz, Alan M. A group II intron RNA is a catalytic component of a DNA endonuclease involved in intron mobility. Cell (Cambridge, Mass.) (1995), 83(4), 529-38.

21. Griffin, Edmund A., Jr.; Qin, Zhifeng; Michels, Williams J., Jr.; Pyle, Anna Marie. Group II intron ribozymes that cleave DNA and RNA linkages with similar efficiency, and lack contacts with substrate 2′-hydroxyl groups. Chem. Biol. (1995), 2(11), 761-70.

22. Michel, Francois; Ferat, Jean Luc. Structure and activities of group II introns. Annu. Rev. Biochem. (1995), 64, 435-61.

23. Abramovitz, Dana L.; Friedman, Richard A.; Pyle, Anna Marie. Catalytic role of 2′-hydroxyl groups within a group II intron active site. Science (Washington, D.C.) (1996), 271(5254), 1410-13.

24. Daniels, Danette L.; Michels, William J., Jr.; Pyle, Anna Marie. Two competing pathways for self-splicing by group II introns: a quantitative analysis of in vitro reaction rates and products. J. Mol. Biol. (1996), 256(1), 31-49.

25. Guo, Hans C. T.; Collins, Richard A. Efficient trans-cleavage of a stem-loop RNA substrate by a ribozyme derived from Neurospora VS RNA. EMBO J. (1995), 14(2), 368-76.

26. Scott, W. G., Finch, J. T., Aaron, K. The crystal structure of an all RNA hammerhead ribozyme: A proposed mechanism for RNA catalytic cleravage. Cell, (1995), 81, 991-1002.

27. McKay, Structure and Function of the Hammerhead ribozyme: an unfinished story. RNA, (1996), 2, 395-403.

28. Long, D., Uhlenbeck, O., Hertel, K. Ligation with hammerhead ribozymes. U.S. Pat. No. 5,633,133.

29. Hertel, K. J., Herschlag, D., Uhlenbach, O. A kinetic and thermodynamic framework for the hammerhead ribozyme reaction. Biochemistry, (1994), 33, 3374-3385. Beigelman, L., et al., Chemical modifications of hammerhead ribozymes. J. Biol. Che., (1995) 270, 25702-25708.

30. Beigelman, L., et al., Chemical modifications of hammerhead ribozymes. J. Biol. Che., (1995) 270, 25702-25708.

31. Hampel, Arnold; Tritz, Richard; Hicks, Margaret; Cruz, Phillip. ‘Hairpin’ catalytic RNA model: evidence for helixes and sequence requirement for substrate RNA. Nucleic Acids Res. (1990), 18(2), 299-304.

32. Chowrira, Bharat M.; Berzal-Herranz, Alfredo; Burke, John M. Novel guanosine requirement for catalysis by the hairpin ribozyme. Nature (London) (1991), 354(6351), 320-2.

33. Berzal-Herranz, Alfredo; Joseph, Simpson; Chowrira, Bharat M.; Butcher, Samuel E.; Burke, John M. Essential nucleotide sequences and secondary structure elements of the hairpin ribozyme. EMBO J. (1993), 12(6), 2567-73.

34. Joseph, Simpson; Berzal-Herranz, Alfredo; Chowrira, Bharat M.; Butcher, Samuel E. Substrate selection rules for the hairpin ribozyme determined by in vitro selection, mutation, and analysis of mismatched substrates. Genes Dev. (1993), 7(1), 130-8.

35. Berzal-Herranz, Alfredo; Joseph, Simpson; Burke, John M. In vitro selection of active hairpin ribozymes by sequential RNA-catalyzed cleavage and ligation reactions. Genes Dev. (1992), 6(1), 129-34.

36. Hegg, Lisa A.; Fedor, Martha J. Kinetics and Thermodynamics of Intermolecular Catalysis by Hairpin Ribozymes. Biochemistry (1995), 34(48), 15813-28.

37. Grasby, Jane A.; Mersmann, Karin; Singh, Mohinder; Gait, Michael J. Purine Functional Groups in Essential Residues of the Hairpin Ribozyme Required for Catalytic Cleavage of RNA. Biochemistry (1995), 34(12), 4068-76.

38. Schmidt, Sabine; Beigelman, Leonid; Karpeisky, Alexander; Usman, Nassim; Sorensen, Ulrik S.; Gait, Michael J. Base and sugar requirements for RNA cleavage of essential nucleoside residues in internal loop B of the hairpin ribozyme: implications for secondary structure. Nucleic Acids Res. (1996), 24(4), 573-81.

39. Perrotta, Anne T.; Been, Michael D. Cleavage of oligoribonucleotides by a ribozyme derived from the hepatitis delta. virus RNA sequence. Biochemistry (1992), 31(1), 16-21.

40. Perrotta, Anne T.; Been, Michael D. A pseudoknot-like structure required for efficient self-cleavage of hepatitis delta virus RNA. Nature (London) (1991), 350(6317), 434-6.

41. Puttaraju, M.; Perrotta, Anne T.; Been, Michael D. A circular trans-acting hepatitis delta virus ribozyme. Nucleic Acids Res. (1993), 21(18), 4253-8.

TABLE II
A. 2.5 μmol Synthesis Cycle ABI 394 Instrument

| | | | Wait Time | Wait Time* | Wait Time |
|---|---|---|---|---|---|
| Reagent | Equivalents | Amount | *DNA | 2′-O-methyl | *RNA |
| Phosphoramidites | 6.5 | 163 μL | 45 sec | 2.5 min | 7.5 min |
| S-Ethyl Tetrazole | 23.8 | 238 μL | 45 sec | 2.5 min | 7.5 min |
| Acetic Anhydride | 100 | 233 μL | 5 sec | 5 sec | 5 sec |
| N-Methyl Imidazole | 186 | 233 μL | 5 sec | 5 sec | 5 sec |
| TCA | 176 | 2.3 mL | 21 sec | 21 sec | 21 sec |
| Iodine | 11.2 | 1.7 mL | 45 sec | 45 sec | 45 sec |
| Beaucage | 12.9 | 645 μL | 100 sec | 300 sec | 300 sec |
| Acetonitrile | NA | 6.67 MI | NA | NA | NA |

B. 0.2 μmol Synthesis Cycle ABI 394 Instrument

| | | | Wait Time | Wait Time | Wait Time |
|---|---|---|---|---|---|
| Reagent | Equivalents | Amount | *DNA | 2′-O-methyl | RNA |
| Phosphoramidites | 15 | 31 μL | 45 sec | 233 sec | 465 sec |
| S-Ethyl Tetrazole | 38.7 | 31 μL | 45 sec | 233 min | 465 sec |
| Acetic Anhydride | 655 | 124 μL | 5 sec | 5 sec | 5 sec |
| N-Methyl Imidazole | 1245 | 124 μL | 5 sec | 5 sec | 5 sec |
| TCA | 700 | 732 μL | 10 sec | 10 sec | 10 sec |
| Iodine | 20.6 | 244 μL | 15 sec | 15 sec | 15 sec |
| Beaucage | 7.7 | 232 μL | 100 sec | 100 sec | 100 sec |
| Acetonitrile | NA | 2.64 mL | NA | NA | NA |

C. 0.2 μmol Synthesis Cycle 96 well Instrument

| | Equivalents | Amount | Wait Time | Wait Time | Wait Time |
|---|---|---|---|---|---|
| Reagent | DNA/2′-O-methyl/Ribo | DNA/2′-O-methyl/Ribo | * DNA | * 2′-O-methyl | *Ribo |
| Phosphoramidites | 22/33/66 | 40/60/120 μL | 60 sec | 180 sec | 360 sec |
| S-Ethyl Tetrazole | 70/105/210 | 40/60/120 μL | 60 sec | 180 sec | 360 sec |
| Acetic An hydride | 265/265/265 | 50/50/50 μL | 10 sec | 10 sec | 10 sec |
| N-Methyl Imidazole | 502/502/502 | 50/50/50 μL | 10 sec | 10 sec | 10 sec |
| TCA | 238/475/475 | 250/500/500 μL | 15 sec | 15 sec | 15 sec |
| Iodine | 6.8/6.8/6.8 | 80/80/80 μL | 30 sec | 30 sec | 30 sec |
| Beaucage | 34/51/51 | 80/120/120 μL | 100 sec | 200 sec | 200 sec |
| Acetonitrile | NA | 1150/1150/1150 μL | NA | NA | NA |

* Wait time does not include contact time during delivery.

TABLE III
HCV DNAzyme and Substrate Sequence

| | | | | |
|---|---|---|---|---|
| Pos | Substrate | Seq ID | DNAzyme | Seq ID |

| | | | | |
|---|---|---|---|---|
| 10 | UGGGGGCG A CACUCCAC | 1 | GTGGAGTG GGCTAGCTACAACGA CGCCCCCA | 4798 |
| 12 | GGGGCGAC A CUCCACCA | 2 | TGGTGGAG GGCTAGCTACAACGA GTCGCCCC | 4799 |
| 27 | GACACUCC A CCAUAGAU | 3 | ATCTATGG GGCTAGCTACAACGA CGAGTGTC | 4800 |
| 20 | ACUCCACC A UAGAUCAC | 4 | GTGATCTA GGCTAGCTACAACGA GGTGGAGT | 4801 |
| 24 | CACCAUAG A UCACUCCC | 5 | GGGAGTGA GGCTAGCTACAACGA CTATGGTG | 4802 |
| 27 | CAUAGAUC A CUCCCCUG | 6 | CAGGGGAG GGCTAGCTACAACGA GATCTATG | 4802 |
| 35 | ACUCCCCU G UGAGGAAC | 7 | GTTCCTCA GGCTAGCTACAACGA AGGGGAGT | 4804 |
| 42 | UGUGAGGA A CUACUGUC | 8 | GACAGTAG GGCTAGCTACAACGA TCCTCACA | 4805 |
| 45 | GACGAACU A CUGUCUUC | 9 | GAAGACAG GGCTAGCTACAACGA AGTTCCTC | 4806 |
| 48 | GAACUACU G UCUUCACG | 10 | CGTGAAGA GGCTAGCTACAACGA AGTAGTTC | 4807 |
| 54 | CUGUCUUC A CGCAGAAA | 11 | TTTCTGCG GGCTAGCTACAACGA GAAGACAG | 4808 |
| 56 | GUCUUCAC G CAGAAAGC | 12 | GCTTTCTG GGCTAGCTACAACGA GTGAAGAC | 4809 |
| 63 | CGCAGAAA G CGUCUAGG | 13 | GCTAGACG GGCTAGCTACAACGA TTTCTGCG | 4810 |
| 65 | CAGAAAGC G UCUAGCCA | 14 | TGGCTAGA GGCTAGCTACAACGA GCTTTCTG | 4811 |
| 70 | AGCGUCUA G CCAUGGCG | 15 | CGCCATGG GGCTAGCTACAACGA TAGACGCT | 4812 |
| 73 | GUCUAGCC A UGGCGUUA | 16 | TAACGCGA GGCTAGCTACAACGA GGCTAGAC | 4813 |
| 76 | UAGCCAUG G CGUUAGUA | 17 | TACTAACG GGCTAGCTACAACGA CATGGCTA | 4814 |
| 78 | GCCAUGGC G UUAGUAUG | 18 | CATACTAA GGCTAGCTACAACGA GCCATGGC | 4815 |
| 82 | UGGCGUUA G UAUGAGUG | 19 | CACTCATA GGCTAGCTACAACGA TAACGCCA | 4816 |
| 84 | GCGUUAGU A UGAGUGUC | 20 | GACACTCA GGCTAGCTACAACGA ACTAACGC | 4817 |
| 88 | UAGUAUGA G UGUCGUGC | 21 | GCACGACA GGCTAGCTACAACGA TCATACTA | 4818 |
| 90 | GUAUGAGU G UCGUGCAC | 22 | CTGCACGA GGCTAGCTACAACGA ACTCATAC | 4819 |
| 93 | UCAGUGUC G UGCAGCCU | 23 | AGGCTGCA GGCTAGCTACAACGA CACACTCA | 4820 |
| 95 | ACUCUCCU G CAGCCUCC | 24 | CCACCCTC GGCTAGCTACAACGA ACGACACT | 4821 |
| 98 | GUCGUGCA G CCUCCAGC | 25 | CCTCGAGG GGCTAGCTACAACGA TGCACGAC | 4822 |
| 107 | CCUCCACC A CCCCCCCU | 26 | ACCCCCCC GGCTAGCTACAACGA CCTGGAGC | 4823 |
| 125 | CCGGGAGA G CCAUAGUG | 27 | CACTATGG GGCTAGCTACAACGA TCTCCCGG | 4824 |
| 128 | GGAGAGCC A UAGUGGUC | 28 | GACCACTA GGCTAGCTACAACGA GGCTCTCC | 4825 |
| 131 | GAGCCAUA G UGGUCUGC | 29 | GCAGACGA GGCTAGCTACAACGA TATGGCTC | 4826 |
| 134 | CCAUACUC G UCUGCGGA | 30 | TCCCCAGA GCCTAGCTACAACGA CACTATGG | 4827 |
| 138 | AGUGGUCU G CGGAACCG | 31 | CGGTTCCG GGCTAGCTACAACGA AGACCACT | 4828 |
| 143 | UCUGCGGA A CCGGUGAG | 32 | CTCACCGG GGCTAGCTACAACGA TCCGCAGA | 4829 |
| 147 | CGGAACCG G UGAGUACA | 33 | TGTACTCA GGCTAGCTACAACGA CGGTTCCG | 4830 |
| 151 | ACCGGUGA G UACACCGG | 34 | CCGGTGTA GGCTAGCTACAACGA TCACCGGT | 4831 |
| 153 | CGCUGAGU A CACCGGAA | 35 | TTCCGGTG GGCTAGCTACAACGA ACTCACCG | 4832 |
| 155 | GUGAGUAC A CCCGAAUU | 36 | AATTCCGG GGCTAGCTACAACGA GTACTCAC | 4833 |
| 161 | ACACCGGA A UUGCCAGG | 37 | CCTGGCAA GGCTAGCTACAACGA TCCGGTGT | 4834 |
| 164 | CCGGAAUU G CCAGGACG | 38 | CGTCCTGG GGCTAGCTACAACGA AATTCCGG | 4835 |
| 170 | UUCCCAGG A CGACCGGG | 39 | CCCGGTCG GGCTAGCTACAACGA CCTCGCAA | 4836 |
| 173 | CCAGGACG A CCGGGUCC | 40 | GGACCCGG GGCTAGCTACAACGA CGTCCTGG | 4837 |
| 178 | ACGACCGG G UCCUUUCU | 41 | AGAAAGGA GGCTAGCTACAACGA CCGGTCGT | 4838 |
| 190 | UUUCUUGC A UCAACCCC | 42 | CGGCTTGA GGCTAGCTACAACGA CCAAGAAA | 4839 |
| 194 | UUGGAUCA A CCCGCUCA | 43 | TGACCGGG GGCTAGCTACAACGA TGATCCAA | 4840 |
| 198 | AUCAACCC G CUCAAUCC | 44 | CCATTCAG GGCTAGCTACAACGA GGCTTGAT | 4841 |
| 203 | CCCGCUCA A UCCCUCCA | 45 | TCCAGGCA GGCTAGCTACAACGA TGAGCCCG | 4842 |
| 205 | CGCUCAAU G CCUCCAGA | 46 | TCTCCACG GGCTAGCTACAACGA ATTCAGCG | 4843 |
| 213 | GCCUGGAG A UUUGCGCG | 47 | CGCCCAAA GGCTAGCTACAACGA CTCCAGGC | 4844 |
| 219 | AGAUUUGG G CGUGCCCC | 48 | GGGGCACC GGCTAGCTACAACGA CCAAATCT | 4845 |
| 221 | AUUUGGGC G UGCCCCCG | 49 | CGGGGCGA GGCTAGCTACAACGA GCCCAAAT | 4846 |
| 223 | UUGGGCCU G CCCCCGCG | 50 | CGCCCCCC GGCTAGCTACAACGA ACCCCCAA | 4847 |
| 229 | GUGCCCCC G CCACACUC | 51 | CAGTCTCC GGCTAGCTACAACGA GCCCCCAC | 4848 |
| 234 | CCCGCGAG A CUGCUAGC | 52 | GCTAGCAG GGCTAGCTACAACGA CTCGCGGC | 4849 |
| 237 | GCCACACU G CUAGCCGA | 53 | TCGGCTAG GGCTAGCTACAACGA AGTCTCGC | 4850 |
| 241 | GACUGCUA G CCGAGUAG | 54 | CTACTCGG GGCTAGCTACAACGA TAGCAGTC | 4851 |
| 246 | CUAGCCGA G UAGUGUUG | 55 | CAACACTA GGCTAGCTACAACGA TCGGCTAG | 4852 |
| 249 | GCCGAGUA G UGUUGGGU | 56 | ACCCAACA GGCTAGCTACAACGA TACTCGGC | 4853 |
| 251 | CGAGUAGU G UUGGGUCG | 57 | CGACCCAA GGCTAGCTACAACGA ACTACTCG | 4854 |
| 256 | AGUGUUGG G UCGCGAAA | 58 | TTTCGCGA GGCTAGCTACAACGA CCAAACACT | 4855 |
| 259 | GUUGGGUC G CGAAAGGC | 59 | GCCTTTCG GGCTAGCTACAACGA GACCCAAC | 4856 |
| 266 | CGCGAAAG G CCUUGUGG | 60 | CCACAAGG GGCTAGCTACAACGA CTTTCGCG | 4857 |
| 271 | AAGGCCUU G UGGUACUG | 61 | CAGTACGA GGCTAGCTACAACGA AAGGCCTT | 4858 |
| 274 | GCCUUGUG G UACUGCCU | 62 | AGGCAGTA GGCTAGCTACAACGA CACAAGGC | 4859 |
| 276 | CUUGUGGU A CCUCCUGA | 63 | TCAGGCAG GGCTAGCTACAACGA ACCACAAG | 4860 |
| 279 | GUGGUACU G CCUGAUAG | 64 | CTATCAGG GGCTAGCTACAACGA AUTACCAC | 4861 |
| 284 | ACUGCCUG A UAGGGUGC | 65 | GCACCCTA GGCTAGCTACAACGA CAGGCAGT | 4862 |
| 289 | CUGAUAGG G UGCUUGCG | 66 | CGCAAGCA GGCTAGCTACAACGA CCTATCAG | 4863 |
| 291 | GAUAGGGU G CUUGCGAG | 67 | CTCGCAAG GGCTAGCTACAACGA ACCCTATC | 4864 |
| 295 | GGGUGCUU G CGAGUGCC | 68 | GGCACTCG GGCTAGCTACAACGA AAGCACCC | 4865 |
| 299 | GCUUGCGA G UGCCCCGG | 69 | CCGGGGCA GGCTAGCTACAACGA TCGCAAGC | 4866 |
| 301 | UUGCGAGU G CCCCGGGA | 70 | TCCCGGGG GGCTAGCTACAACGA ACTCGCAA | 4867 |
| 311 | CCCGGGAG G UCUCGUAG | 71 | CTACGAGA GGCTAGCTACAACGA CTCCCCGG | 4868 |
| 316 | GAGGUCUC G UAGACCGU | 72 | ACGGTCTA GGCTAGCTACAACGA GAGACCTC | 4869 |
| 320 | UCUCGUAG A CCGUGCAC | 73 | GTGCACGG GGCTAGCTACAACGA CTACGAGA | 4870 |
| 323 | CGUAGACC G UGCACCAU | 74 | ATGGTGCA GGCTAGCTACAACGA GGTCTACG | 4871 |
| 325 | UAGACCGU G CACCAUGA | 75 | TCATGGTG GGCTAGCTACAACGA ACGGTCTA | 4872 |
| 327 | GACCGUGC A CCAUCACC | 76 | GCTCATGG GGCTAGCTACAACGA GCACGGTC | 4873 |
| 330 | CGUGCACC A UGAGCACG | 77 | CGTGCTCA GGCTAGCTACAACGA GGTGCACG | 4874 |
| 334 | CACCAUGA G CACGAAUC | 78 | GATTCGTG GGCTAGCTACAACGA TCATGGTG | 4875 |
| 336 | CCAUGAGC A CGAAUCCU | 79 | AGGATTCG GGCTAGCTACAACGA GCTCATGG | 4876 |
| 340 | GAGCACCA A UCCUAAAC | 80 | GTTTAGGA GGCTAGCTACAACGA TCGTGCTC | 4877 |
| 347 | AAUCCUAA A CCUCAAAG | 81 | CTTTGAGC GGCTAGCTACAACGA TTAGGATT | 4878 |
| 360 | AAAGAAAA A CCAAACGU | 82 | ACGTTTGG GGCTAGCTACAACGA TTTTCTTT | 4879 |
| 365 | AAAACCAA A CGUAACAC | 83 | GTGTTACG GGCTAGCTACAACGA TTGGTTTT | 4880 |
| 367 | AACCAAAC G UAACACCA | 84 | TGGTGTTA GGCTAGCTACAACGA GTTTGGTT | 4881 |
| 370 | CAAACGUA A CACCAACC | 85 | GCTTGCTC GGCTAGCTACAACGA TACGTTTG | 4882 |
| 372 | AACGUAAC A CCAACCGC | 86 | GCGGTTGG GGCTAGCTACAACGA GTTACCTT | 4883 |
| 376 | UAACACCA A CCGCCGCC | 87 | GGCGGCGG GGCTAGCTACAACGA TGGTGTTA | 4884 |
| 379 | CACCAACC G CCGCCCAC | 88 | GTGCGCGC GGCTAGCTACAACGA CCTTCCTC | 4885 |
| 382 | CAACCGCC G CCCACACG | 89 | CCTCTCGG GGCTAGCTACAACGA GGCGGTTG | 4886 |
| 386 | CGCCGCCC A CACGACGU | 90 | ACCTCCTG GGCTAGCTACAACGA GGGCGGCC | 4887 |
| 391 | CCCACAGG A CGUCAAGU | 91 | ACTTGACC GGCTAGCTACAACGA CCTGTGGC | 4888 |
| 393 | CACAGGAC G UCAAGUUC | 92 | CAACTTGA GGCTAGCTACAACGA GTCCTGTG | 4889 |
| 398 | CACGUCAA G UUCCCGCG | 93 | CCCCGCAA GGCTAGCTACAACGA TTGACCTC | 4890 |
| 406 | GUUCCCGG G CCCUCCUC | 94 | CACCACCC GGCTAGCTACAACGA CCCCCAAC | 4891 |
| 409 | CCCGGGCC G UCCUCACA | 95 | TCTGACGA GGCTAGCTACAACGA CCCCCGCG | 4892 |
| 412 | GGGCGGUG G UCAGAUCG | 96 | CGATCTGA GGCTAGCTACAACGA CACCGCCC | 4893 |
| 417 | GUGGUCAG A UCCUUGCU | 97 | ACCAACGA GGCTAGCTACAACGA CTGACCAC | 4894 |
| 420 | CUCAGAUC G UUCGUGGA | 98 | TCCACCAA GGCTAGCTACAACGA GATCTGAC | 4895 |
| 424 | CAUCGUUG G UGGAGUUU | 99 | AAACTCGA GGCTAGCTACAACGA CAACGATC | 4896 |
| 429 | UUGGUCGA G UUUACCUC | 100 | CACCTAAA GGCTAGCTACAACGA TCCACCAA | 4897 |
| 433 | UGGAGUUU A CCUGUUGC | 101 | CCAACAGC GGCTAGCTACAACGA AAACTCCA | 4898 |
| 437 | GUUUACCU G UUCCCGCG | 102 | CGCGGCAA GGCTAGCTACAACGA ACGTAAAC | 4899 |
| 440 | UACCUCUU G CCCCGCAG | 103 | CTCCCCCG GGCTAGCTACAACGA AACACCTA | 4900 |
| 443 | CUCUUCCC G CCCACCCC | 104 | CCCCTGCG GGCTAGCTACAACGA CCCAACAG | 4901 |
| 445 | GUUCCCGC G CAGGGGCC | 105 | GGCCCCTG GGCTAGCTACAACGA GCGGCAAC | 4902 |
| 451 | GCGCACCG G CCCCACGU | 106 | ACCTCGCG GGCTAGCTACAACGA CCCTCCCC | 4903 |
| 458 | CCCCCCAG G UUGGGUGU | 107 | ACACCCAA GGCTAGCTACAACGA CTCGCCCC | 4904 |
| 463 | CACCUUCG G UGUCCGCG | 108 | CGCGCACA GGCTAGCTACAACGA CCAACCTG | 4905 |
| 465 | GCUUGCCU G UCCCCCCC | 109 | CGCGCCGA GGCTAGCTACAACGA ACCCAACC | 4906 |
| 467 | UUGCGUCU G CCCCCCAC | 110 | CTCGCGCG GGCTAGCTACAACGA ACACCCAA | 4907 |
| 469 | GGGUGUGC G CGCGACUA | 111 | TAGTCGCG GGCTAGCTACAACGA GCACACCC | 4908 |
| 471 | GUGUGCGC G CGACUAGG | 112 | CCTAGTCG GGCTAGCTACAACGA GCGCACAC | 4909 |
| 474 | UGCGCGCG A CUAGGAAG | 113 | CTTCCTAG GGCTAGCTACAACGA CGCGCGCA | 4910 |
| 483 | CUAGGAAG A CUUCCGAG | 114 | CTCGGAAG GGCTAGCTACAACGA CTTCCTAG | 4911 |
| 491 | ACUUCCGA G CGGUCGCA | 115 | TGCGACCG GGCTAGCTACAACGA TCGGAAGT | 4912 |
| 494 | UCCGAGCG G UCGCAACC | 116 | GGTTGCGA GGCTAGCTACAACGA CGCTCGGA | 4913 |
| 497 | GAGCGGUC G CAACCUCG | 117 | CGAGGTTG GGCTAGCTACAACGA GACCGCTC | 4914 |
| 500 | CGGUCGCA A CCUCGUGG | 118 | CCACGAGG GGCTAGCTACAACGA TGCGACCG | 4915 |
| 505 | GCAACCUC G UGGAAGGC | 119 | GCCTTCGA GGCTAGCTACAACGA GAGGTTGC | 4916 |
| 512 | CGUGGAAG G CGACAACC | 120 | GGTTGTCG GGCTAGCTACAACGA CTTCCACG | 4917 |
| 515 | GGAAGGCG A CAACCUAU | 121 | ATAGGTTG GGCTAGCTACAACGA CGCCTTCC | 4918 |
| 518 | AGGCGACA A CCUAUCCC | 122 | GGGATAGG GGCTAGCTACAACGA TGTCGCCT | 4919 |
| 522 | GACAACCU A UCCCCAAG | 123 | CTTGGGGA GGCTAGCTACAACGA AGGTTGTC | 4920 |
| 531 | UCCCCA~G G CUCGCCGG | 124 | CCGGCGAG GGCTAGCTACAACGA CTTGGGGA | 4921 |
| 535 | CAAGGCUC G CCGGCCCG | 125 | CGGGCCGG GGCTAGCTACAACGA GAGCCTT3 | 4922 |
| 539 | GCUCGCCG G CCCGAGGG | 126 | CCCTCGGG GGCTAGCTACAACGA CGGCGAGC | 4923 |
| 547 | GCCCGAGG G CAGGGCCU | 127 | AGGCCCTG GGCTAGCTACAACGA CCTCGGGC | 4924 |
| 552 | AGGGCACG G CCUGGCCU | 128 | AGCCCAGG GGCTAGCTACAACGA CCTGCCCT | 4925 |
| 558 | CGGCCUGG G CUCAGCCC | 129 | GGGCTCAG GGCTAGCTACAACGA CCAGGCCC | 4926 |
| 563 | UGGGCUCA G CCCGCGUA | 130 | TACCCGGG GGCTAGCTACAACGA TGAGCCCA | 4927 |
| 569 | CAGCCCGG G UACCCUUG | 131 | CAAGGGTA GGCTAGCTACAACGA CCGCGCTG | 4928 |
| 571 | GCCCGGGU A CCCUUCGC | 132 | GCCAAGGG GGCTAGCTACAACGA ACCCGGGC | 4929 |
| 578 | UACCCUUG G CCCCUCUA | 133 | TAGAGCCC GGCTAGCTACAACGA CAAGGGTA | 4930 |
| 586 | CCCCCUCU A UGGCAAUG | 134 | CATTGCCA GGCTAGCTACAACGA ACAGCGGC | 4931 |
| 589 | CCUCUAUG G CAAUGACG | 135 | CCTCATTG GGCTAGCTACAACGA CATAGAGG | 4932 |
| 592 | CUAUCCCA A UGAGGGCU | 136 | AGCCCTCA GGCTAGCTACAACGA TGCCATAG | 4933 |
| 598 | CAAUGAGC G CUUAGGGU | 137 | ACCCTAAC GGCTAGCTACAACGA CCTCATTG | 4934 |
| 605 | GGCUUAGG G UGCGCACG | 138 | CCTGCCGA GGCTAGCTACAACGA CCTAACCC | 4935 |
| 609 | UAGCCUCG G CACGAUGG | 139 | CCATCCTG GGCTAGCTACAACGA CCACCCTA | 4936 |
| 614 | UCCCCAGC A UCGCUCCU | 140 | ACCAGCGA GGCTAGCTACAACGA CCTGCCCA | 4937 |
| 617 | CCAGGAUG G CUCCUCUC | 141 | GACAGGAC GGCTAGCTACAACGA CATCCTCC | 4938 |
| 623 | UCCCUCCU G UCACCCCC | 142 | CGGGCTGA GGCTAGCTACAACGA AGGACCCA | 4939 |
| 626 | CUCCUGUC A CCCCGCCG | 143 | CCGCCGCC GGCTAGCTACAACGA GACAGGAG | 4940 |
| 631 | GUCACCCC G CCGCUCCC | 144 | GGGACCCG GGCTAGCTACAACGA CGGCTCAC | 4941 |
| 634 | ACCCCGCG G CUCCCCCC | 145 | GCCGCGAG GGCTAGCTACAACGA CGCGGGGT | 4942 |
| 641 | CGCUCCCC G CCUACUUC | 146 | CAACTACC GGCTAGCTACAACGA CGCCACCC | 4943 |
| 646 | CCGCCCUA G UUCGCGCC | 147 | GGCCCCAA GGCTAGCTACAACGA TAGCCCGG | 4944 |
| 652 | UACUUGGG G CCCCACGC | 148 | CCGTCGGG GGCTAGCTACAACGA CCCAACTA | 4945 |
| 657 | GCGGCCCC A CGCACCCC | 149 | GGGGTCCC GGCTAGCTACAACGA GCGGCCCC | 4946 |
| 661 | CCCCACGG A CCCCCCGC | 150 | GCCCCCGG GGCTAGCTACAACGA CCGTGCGG | 4947 |
| 668 | GACCCCCG G CGUAGGUC | 151 | CACCTACG GGCTAGCTACAACGA CCGGGCTC | 4948 |
| 670 | CCCCCGGC G UACGUCGC | 152 | CCGACCTA GGCTAGCTACAACGA GCCGGGGC | 4949 |
| 674 | CGCCCUAG G UCGCCUAA | 153 | TTACCCGA GGCTAGCTACAACGA CTACGCCC | 4950 |
| 677 | CGUAGCUC G CGUAACUU | 154 | AAGTTACG GGCTAGCTACAACGA GACCTACG | 4951 |
| 679 | UACCUCCC G UAACUUCC | 155 | CCAAGTTA GGCTAGCTACAACGA GCGACCTA | 4952 |
| 682 | GUCCCCUA A CUUGCGUA | 156 | TACCCAAC GGCTAGCTACAACGA TACCCCAC | 4953 |
| 688 | UAACUUGG G UAAGGUCA | 157 | TCACCTTA GGCTAGCTACAACGA CCAAGTTA | 4954 |
| 693 | UCGCUAAC G UCAUCCAU | 158 | ATCCATGA GGCTAGCTACAACGA CTTACCCA | 4955 |
| 696 | CUAACCUC A UCCAUACC | 159 | CCTATCGA GGCTAGCTACAACGA CACCTTAC | 4956 |
| 700 | CGUCAUCC A UACCCUCA | 160 | TCACGGTA GGCTAGCTACAACGA CGATGACC | 4957 |
| 702 | UCAUCCAU A CCCUCACA | 161 | TCTCACCC GGCTAGCTACAACGA ATCCATCA | 4958 |
| 708 | AUACCCUC A CAUCCCCC | 162 | CCCCCATC GGCTAGCTACAACGA CACCCTAT | 4959 |
| 710 | ACCCUCAC A UCCCGCUU | 163 | AACCCGCA GGCTAGCTACAACGA GTCACCCT | 4960 |
| 712 | CCUCACAU G CGCCUUCC | 164 | CCAACCCC GGCTAGCTACAACGA ATCTCACC | 4961 |
| 715 | CACAUCCC G CUUCCCCC | 165 | CCCCCAAC GGCTAGCTACAACGA CCCATCTC | 4962 |
| 720 | CCCCCUUC G CCGACCUC | 166 | CACGTCCC GGCTAGCTACAACGA CAACCCCC | 4963 |
| 724 | CUUCCCCC A CCUCAUCC | 167 | CCATCACC GGCTAGCTACAACGA CCCCCAAC | 4964 |
| 729 | CCGACCUC A UGGGGUAC | 168 | GTACCCGA GGCTAGCTACAACGA GAGGTCGG | 4965 |
| 734 | CUCAUGGG G UACAUUCC | 169 | GGAATGTA GGCTAGCTACAACGA CCCATGAG | 4966 |
| 736 | CAUGGGGU A CAUUCCGC | 170 | GCGGAATG GGCTAGCTACAACGA ACCCCATG | 4967 |
| 738 | UGGGGUAC A UUCCGCUC | 171 | GAGCGGAA GGCTAGCTACAACGA GTACCCCA | 4968 |
| 743 | UACAUUCC G CUCGUCGG | 172 | CCGACGAG GGCTAGCTACAAACGA GGAATGTA | 4969 |
| 747 | UUCCGCUC G UCGGCGCC | 173 | GGCGCCGA GGCTAGCTACAACGA GAGCGGAA | 4970 |
| 751 | GCUCGUCG G CGCCCCCU | 174 | AGGGGGCG GGCTAGCTACAACGA CGACGAGC | 4971 |
| 753 | UCGUCGGC G CCCCCUUG | 175 | CAAGGGGC3 GGCTAGCTACAACGA GCCGACGA | 4972 |
| 766 | CUUGGGAG G CACTTGCGA | 176 | TGGCAGTG GGCTAGCTACAACGA CTCCCAAG | 4973 |
| 768 | UGGGAGGC A CUGCCAGG | 177 | CCTGGCAG GGCTAGCTACAACGA GCCTCCCA | 4974 |
| 771 | GAGGCACU G CCAGGGCC | 178 | GGCCCTGG GGCTAGCTACAAACGA AGTGCCTC | 4975 |
| 777 | CUGCCAGG G CCCUGGCG | 179 | CGCCAGGG GGCTAGCTACAACGA CCTCGCAC | 4976 |
| 783 | GGCCCCUG G CGCAUGGC | 180 | GCCATGCG GGCTAGCTACAACGA CAGGGCCC | 4977 |
| 785 | GCCCUGGC G CAUGCCGU | 181 | ACGCCATG GGCTAGCTACAACGA GCCAGGGC | 4978 |
| 787 | CCUCGCGC A UGCCGUCC | 182 | GGACGCGA GGCTAGCTACAACGA GCGCCAGG | 4979 |
| 790 | CGCCCAUG G CGUCCCGG | 183 | CCCGGACG GGCTAGCTACAACGA CATGCGCC | 4980 |
| 792 | CGCAUGGC G UCCGGCUU | 184 | AACCCCGA GGCTAGCTACAACGA CCCATGCG | 4981 |
| 798 | CCGUCCGG G UUCUGCAA | 185 | TTCCACAA GGCTAGCTACAACGA CCGGACCC | 4982 |
| 808 | UCUGGAAG A CCGCGUGA | 186 | TCACGCCG GGCTAGCTACAACGA CTTCCAGA | 4983 |
| 811 | GGAAGACG G CGUCAACU | 187 | AGTTCACC GGCTAGCTACAACGA CCTCTTCC | 4984 |
| 813 | AACACGCC G UGAACUAU | 188 | ATAGTTCA GGCTAGCTACAACGA GCCGTCTT | 4985 |
| 817 | CCCCCUCA A CUAUGCAA | 189 | TTCCATAC GGCTAGCTACAACGA TCACGCCC | 4986 |
| 820 | CGUGAACU A UGCAACAG | 190 | CTGTTCGA GGCTAGCTACAACGA ACTTCACG | 4987 |
| 822 | UGAACUAU G CAACAGCG | 191 | CCCTGTTC GGCTAGCTACAACGA ATACTTCA | 4988 |
| 825 | ACUAUGCA A CAGGGAAU | 192 | ATTCCCTC GGCTAGCTACAACGA TCCATAGT | 4989 |
| 832 | AACAGCCA A UCUCCCCG | 193 | CCCGCAGA GGCTAGCTACAACGA TCCCTGTT | 4990 |
| 836 | CCCAAUCU G CCCCGUUC | 194 | CAACCGCG GGCTAGCTACAACGA AGATTCCC | 4991 |
| 841 | UCUCCCCG G UUGCUCUU | 195 | AACACCAA GGCTAGCTACAACGA CGCGCAGA | 4992 |
| 844 | GCCCGGUU G CUCUUUCU | 196 | ACAAAGAG GGCTAGCTACAACGA AACCCCGC | 4993 |
| 855 | CUUUCUCU A UCUUCCUC | 197 | GACGAAGA GGCTAGCTACAACGA AGAGAAAG | 4994 |
| 867 | UCCUCUUC G CUCUGCUC | 198 | CAGCACAG GGCTAGCTACAACGA CAAGAGGA | 4995 |
| 872 | UUCCCUCU G CUCCCCUC | 199 | CACGGCAG GGCTAGCTACAACGA ACAGCCAA | 4996 |
| 875 | CCUCUGCU G CCCUCUCU | 200 | AGACACGC GGCTAGCTACAACGA AGCAGACC | 4997 |
| 880 | GCUGCCCU G UCUGACCA | 201 | TGGTCACA GGCTAGCTACAACGA AGGGCAGC | 4998 |
| 885 | CCUGUCUC A CCAUCCGA | 202 | TGGGATGC GGCTAGCTACAACGA CACACAGG | 4999 |
| 888 | GUCUGACC A UCCCAGCC | 203 | GGCTGGGA GGCTAGCTACAACGA GGTCAGAC | 5000 |
| 894 | CCAUCCGA G CCUCCGCU | 204 | AGCGGACG GGCTAGCTACAACGA TCGGATGG | 5001 |
| 900 | CAGCCUCC G CUUAUGAC | 205 | CTCATAAG GGCTAGCTACAACGA GGAGGCTG | 5002 |
| 904 | CUCCCCUU A UGACGUCU | 206 | ACACCTCA GGCTAGCTACAACGA AACCCGAG | 5003 |
| 909 | CUUAUGAC G UGUCCAAC | 207 | GTTGCACA GGCTAGCTACAACGA CTCATAAG | 5004 |
| 911 | UAUCAGGU G UCCAACCC | 208 | CCCTTGCA GGCTAGCTACAACGA ACCTCATA | 5005 |
| 913 | UCACCUGU G CAACGCCU | 209 | ACCCCTTC GGCTAGCTACAACGA ACACCTCA | 5006 |
| 916 | CCUCUCCA A CCCCUCCC | 210 | CCCACCCG GGCTAGCTACAACGA TGCACACC | 5007 |
| 918 | UCUCCAAC G CCUCCCCC | 211 | CCCCCACC GGCTAGCTACAACGA CTTCCACA | 5008 |
| 920 | UGCAACCC G UCCCCCCU | 212 | ACCCCCGA GGCTAGCTACAACGA CCCTTCCA | 5009 |
| 926 | CCGUCCCC G CUGUACCA | 213 | TGGTACAC GGCTAGCTACAACGA CCCCACCC | 5010 |
| 929 | UCCCCCCU G UACCAUCU | 214 | ACATCCTA GGCTAGCTACAACGA ACCCCCCA | 5011 |
| 931 | CGCCCUCU A CCAUCUCA | 215 | TGACATCC GGCTAGCTACAACGA ACAGCCCC | 5012 |
| 934 | CCUCUACC A UCUCACCA | 216 | TCCTCACA GGCTAGCTACAACGA GCTACACC | 5013 |
| 936 | UCUACCAU G UCACCAAC | 217 | CTTCCTCA GGCTAGCTACAACGA ATCCTACA | 5014 |
| 939 | ACCAUCUC A CCAACCAU | 218 | ATCCTTCG GGCTAGCTACAACGA CACATCCT | 5015 |
| 943 | UCUCACCA A CGAUUCCU | 219A | CCAATCC GGCTAGCTACAACGA TCCTCACA | 5016 |
| 946 | CACGAACC A UUGCUCGA | 220 | TGCAGCAA GGCTAGCTACAACGA CGTTCCTC | 5017 |
| 949 | CAACCAUU G CUCCAACU | 221 | ACTTCCAC GGCTAGCTACAACGA AATCGTTC | 5018 |
| 955 | UUCCUCCA A CUCAACCA | 222 | TCCTTCAC GGCTAGCTACAACGA TGCACCAA | 5019 |
| 961 | CAACUCAA G CAUUCUCU | 223 | ACACAATC GGCTAGCTACAACGA TTGAGTTC | 5020 |
| 963 | ACUCAACC A UUGUGUAU | 224 | ATACACAA GGCTAGCTACAACGA CCTTGAGT | 5021 |
| 966 | CAAGCAUU G UGUAUGAG | 225 | CTCATACA GGCTAGCTACAACGA AATGCTTG | 5022 |
| 968 | AGCAUUGU G UAUGAGGC | 226 | GCCTCATA GGCTAGCTACAACGA ACAATGCT | 5023 |
| 970 | CAUUGUGU A UGAGGCAG | 227 | CTGCCTCA GGCTAGCTACAACGA ACACAATG | 5024 |
| 975 | UGUAUGAG G CACAGGAC | 228 | GTCCTCTG GGCTAGCTACAACGA CTCATACA | 5025 |
| 982 | GGCAGAGG A CAUGAUCA | 229 | TGATCATG GGCTAGCTACAACGA CCTCTGCC | 5026 |
| 984 | CAGAGGAC A UGAUCAUG | 230 | CATGATCA GGCTAGCTACAACGA GTCCTCTG | 5027 |
| 987 | AGGACAUG A UCAUGCAC | 231 | GTGCATGA GGCTAGCTACAACGA CATGTCCT | 5028 |
| 990 | ACAUGAUC A UGCACACC | 232 | GGTGTGCA GGCTAGCTACAACGA GATCATGT | 5029 |
| 992 | AUGAUCAU G CACACCCC | 233 | GGGGTGTG GGCTAGCTACAACGA ATGATCAT | 5030 |
| 994 | GAUCAUGC A CACCCCGG | 234 | CCGGGGTG GGCTAGCTACAACGA GCATGATC | 5031 |
| 996 | UCAUGCAC A CCCCGGGG | 235 | CCCCGGGG GGCTAGCTACAACGA GTGCATGA | 5032 |
| 1004 | ACCCCGGG G UGCGUGCC | 236 | GGCACGCA GGCTAGCTACAACGA CCCGGGGT | 5033 |
| 1006 | CCCGGGGU G CGUGCCCU | 237 | AGGGCACG GGCTAGCTACAACGA ACCCCGGG | 5034 |
| 1008 | CGGGGUGC G UGCCCUGC | 238 | GCAGGGCA GGCTAGCTACAACGA GCACCCCG | 5035 |
| 1010 | GGGUCCCU G CCCUCCGU | 239 | ACCCACCC GGCTAGCTACAACGA ACCCACCC | 5036 |
| 1015 | CCUGCCCU G CCUUCGCG | 240 | CCCGAACG GGCTAGCTACAACGA ACGGCACG | 5037 |
| 1017 | UGCCCUGC G UUCGGGAC | 241 | CTCCCCAA GGCTAGCTACAACGA GCAGGGCA | 5038 |
| 1027 | UCGGGAGA A CAACUCCU | 242 | AGGAGTTG GGCTAGCTACAACGA TCTCCCGA | 5039 |
| 1030 | GGAGAACA A CUCCUCCC | 243 | GGGAGGAG GGCTAGCTACAACGA TGTTCTCC | 5040 |
| 1039 | CUCCUCCC G CUGCUGGG | 244 | CCCAGCAG GGCTAGCTACAACGA GGGAGGAG | 5041 |
| 1042 | CUCCCGCU G CUGGGUAG | 245 | CTACCCAG GGCTAGCTACAACGA AGCGGGAG | 5042 |
| 1047 | GCUGCUGG G UAGCGCUC | 246 | GAGCGCTA GGCTAGCTACAACGA CCAGCAGC | 5043 |
| 1050 | GCUGGGUA G CGCUCACU | 247 | AGTGAGCG GGCTAGCTACAACGA TACCCAGC | 5044 |
| 1052 | UGGGUAGC G CUCACUCC | 248 | GGAGTGAG GGCTAGCTACAACGA GCTACCCA | 5045 |
| 1056 | UAGCGCUC A CUCCCACG | 249 | CGTGGGAG GGCTAGCTACAACGA GAGCGCTA | 5046 |
| 1062 | UCACUCCC A CGCUCGCG | 250 | CGCGAGCG GGCTAGCTACAACGA GGGAGTGA | 5047 |
| 1064 | ACUCCCAC G CUCGCGGC | 251 | GCCGCGAG GGCTAGCTACAACGA GTGGGAGT | 5048 |
| 1068 | CCACGCUC G CGGCCAGG | 252 | CCTGGCCG GGCTAGCTACAACGA GAGCGTGG | 5049 |
| 1071 | CGCUCGCG G CCAGGAAU | 253 | ATTCCTGG GGCTAGCTACAACGA CGCGAGCG | 5050 |
| 1078 | GGCCAGGA A UGCCAGCA | 254 | TGCTGGCA GGCTAGCTACAACGA TCCTGGCC | 5051 |
| 1080 | CCAGGAAU G CCAGCAUC | 255 | GATGCTGG GGCTAGCTACAACGA ATTCCTGG | 5052 |
| 1084 | GAAUGCGA G CAUCCCGA | 256 | TGGGGATG GGCTAGCTACAACGA TGGCATTC | 5053 |
| 1086 | AUGCCAGC A UCCCCACU | 257 | AGTGGGGA GGCTAGCTACAACGA GCTGGCAT | 5054 |
| 1092 | GCAUCCCC A CUACGACG | 258 | CGTCGTAG GGCTAGCTACAACGA GGGGATGC | 5055 |
| 1095 | UCCCCACU A CGACGAUA | 259 | TATCGTCG GGCTAGCTACAACGA AGTGGGGA | 5056 |
| 1098 | CCACUACG A CGAUACGG | 260 | CCGTATCG GGCTAGCTACAACGA CGTAGTGG | 5057 |
| 1101 | CUACGACG A UACGGCGU | 261 | ACGCCGTA GGCTAGCTACAACGA CGTCGTAG | 5058 |
| 1103 | ACGACGAU A CGGCGUCA | 262 | TGACGCCG GGCTAGCTACAACGA ATCGTCGT | 5059 |
| 1106 | ACGAUACG G CGUCACGU | 263 | ACGTGACG GGCTAGCTACAACGA CGTATCGT | 5060 |
| 1108 | GAUACGGC G UCACGUCG | 264 | CGACGTGA GGCTAGCTACAACGA GCCGTATC | 5061 |
| 1111 | ACGGCGUC A CGUCGAUU | 265 | AATCGACG GGCTAGCTACAACGA GACGCCGT | 5062 |
| 1113 | GGCGUCAC G UCGAUUUG | 266 | CAAATCGA GGCTAGCTACAACGA GTGACGCC | 5063 |
| 1117 | UCACGUCG A UUUGCUCG | 267 | CGAGCAAA GGCTAGCTACAACGA CGACGTGA | 5064 |
| 1121 | GUCGAUUU G CUCGUUGG | 268 | CCAACGAG GGCTAGCTACAACGA AAATCGAC | 5065 |
| 1125 | AUUUGCUC G UUGGGGCG | 269 | CGCCCCAA GGCTAGCTACAACGA GACCAAAT | 5066 |
| 1131 | UCGUUCCG G CGGCUGCU | 270 | ACCACCCG GGCTAGCTACAACGA CCCAACGA | 5067 |
| 1134 | UUGCGGCG G CUGCUUUC | 271 | CAAACCAG GGCTAGCTACAACGA CCCCCCAA | 5068 |
| 1137 | GGGCGGCU G CUUUCUGC | 272 | GCACAAAC GGCTAGCTACAACGA AGCCGCCC | 5069 |
| 1144 | UCCUUUCU G CUCUGCUA | 273 | TACCACAC GGCTAGCTACAACGA ACAAACCA | 5070 |
| 1149 | UCUCCUCU G CUAUCUAC | 274 | CTACATAC GGCTAGCTACAACGA ACACCACA | 5071 |
| 1152 | CCUCUCCU A UCUACCUC | 275 | CACCTACA GGCTAGCTACAACGA ACCACACC | 5072 |
| 1154 | UCUCCUAU G UACCUGCG | 276 | CCCACCTA GGCTAGCTACAACGA ATACCACA | 5073 |
| 1156 | UCCUAUCU A CGUGGGGG | 277 | CCCCCACG GGCTAGCTACAACGA ACATACCA | 5074 |
| 1158 | CUAUCUAC G UGCGCGAU | 278 | ATCCCCGA GGCTAGCTACAACGA CTACATAC | 5075 |
| 1165 | CCUCCCCC A UCUCUGCG | 279 | CCCACACA GGCTAGCTACAACGA CCCCCACC | 5076 |
| 1171 | GGAUCUCU G CGGAUCUG | 280 | CAGATCCG GGCTAGCTACAACGA ACACATCC | 5077 |
| 1175 | CUCUGCGG A UCUGUCUU | 281 | AAGACAGA GGCTAGCTACAACGA CCCCACAG | 5078 |
| 1179 | GCGGAUCU G UCUUCCUC | 282 | GAGGAAGA GGCTAGCTACAACGA AGATCCGC | 5079 |
| 1188 | UCUUCCUC G UCUCUCAG | 283 | CTGAGAGA GGCTAGCTACAACGA GAGGAAGA | 5080 |
| 1196 | GUCUCUCA G CUGUUCAC | 284 | GTGAACAG GGCTAGCTACAACGA TGAGAGAC | 5081 |
| 1199 | UCUCAGCU G UUCACCUU | 285 | AAGGTGAA GGCTAGCTACAACGA AGCTGAGA | 5082 |
| 1203 | AGCUGUUC A CCUUCUCG | 286 | CGAGAAGG GGCTAGCTACAACGA GAACAGCT | 5083 |
| 1211 | ACCUUCUC G CCUCGCCG | 287 | CGGCGAGG GGCTAGCTACAACGA GAGAAGCT | 5084 |
| 1216 | CUCGCCUC G CCGGUAUG | 288 | CATACCGG GGCTAGCTACAACGA GAGGCGAG | 5085 |
| 1220 | CCUCGCCG G UAUGAGAC | 289 | GTCTCATA GGCTAGCTACAACGA CGGCGAGG | 5086 |
| 1222 | UCGCCGGU A UGAGACAG | 290 | CTGTCTCA GGCTAGCTACAACGA ACCGGCGA | 5087 |
| 1227 | GGUAUGAG A CAGUACAG | 291 | CTGTACTG GGCTAGCTACAACGA CTCATACC | 5088 |
| 1230 | AUGAGACA G UACAGGAC | 292 | GTCCTGTA GGCTAGCTACAACGA TGTCTCAT | 5089 |
| 1232 | GAGACAGU A CAGGACUG | 293 | CAGTCCTG GGCTAGCTACAACGA ACTGTCTC | 5090 |
| 1237 | AGUACAGG A CUGUAAUU | 294 | AATTACAG GGCTAGCTACAACGA CCTGTACT | 5091 |
| 1240 | ACAGGACU G UAAUUGCU | 295 | AGCAATTA GGCTAGCTACAACGA AGTCCTGT | 5092 |
| 1243 | GGACUGUA A UUGCUCGA | 296 | TCGAGCAA GGCTAGCTACAACGA TACAGTCC | 5093 |
| 1246 | CUGUAAUU G CUCGAUCU | 297 | AGATCGAG GGCTAGCTACAACGA AATTACAG | 5094 |
| 1251 | AUUGCUCG A UCUAUCCC | 298 | GGGATAGA GGCTAGCTACAACGA CGAGCAAT | 5095 |
| 1255 | CUCGAUCU A UCCCGGCC | 299 | GGCCGGGA GGCTAGCTACAACGA AGATCGAG | 5096 |
| 1261 | CUAUCCCG G CCACGUAU | 300 | ATACGTGG GGCTAGCTACAACGA CGGGATAG | 5097 |
| 1264 | UCCCGGCC A CGUAUCAG | 301 | CTGATACG GGCTAGCTACAACGA GGCCGGGA | 5098 |
| 1266 | CCGGCCAC G UAUCAGGC | 302 | GCCTGATA GGCTAGCTACAACGA GTGGCCGG | 5099 |
| 1268 | GGCCACGU A UCAGGCGA | 303 | TGGCCTGA GGCTAGCTACAACGA ACGTGGCC | 5100 |
| 1273 | CGUAUCAG G CCAUCGCA | 304 | TGCGATGG GGCTAGCTACAACGA CTGATACG | 5101 |
| 1276 | AUCAGGCC A UCGCAUGG | 305 | CCATGCGA GGCTAGCTACAACGA GGCCTGAT | 5102 |
| 1279 | AGGCCAUC G CAUGGCUU | 306 | AAGCCATG GGCTAGCTACAACGA GATGGCCT | 5103 |
| 1281 | GCCAUCGC A UGGCUUGG | 307 | CCAAGCGA GGCTAGCTACAACGA GCGATGGC | 5104 |
| 1284 | AUCGCAUG G CUUGGGAU | 308 | ATCCCAAG GGCTAGCTACAACGA CATGCGAT | 5105 |
| 1291 | GGCUUGGG A UAUGAUGA | 309 | TCATCATA GGCTAGCTACAACGA CCCAAGCC | 5106 |
| 1293 | CUUGGGAU A UGAUGAUG | 310 | CATCATCA GGCTAGCTACAACGA ATCCCAAG | 5107 |
| 1296 | GGGAUAUG A UGAUGAAU | 311 | ATTCATCA GGCTAGCTACAACGA CATATCCC | 5108 |
| 1299 | AUAUGAUG A UGAAUUGG | 312 | CCAATTCA GGCTAGCTACAACGA CATCATAT | 5109 |
| 1303 | GAUGAUGA A UUGGUCAC | 313 | GTGACCAA GGCTAGCTACAACGA TCATCATC | 5110 |
| 1307 | AUGAAUUG G UCACCUAC | 314 | GTAGGTGA GGCTAGCTACAACGA CAATTCAT | 5111 |
| 1310 | AAUUGGUC A CCUACAAC | 315 | GTTGTAGG GGCTAGCTACAACGA GACCAATT | 5112 |
| 1314 | GGUCACCU A CAACAGCC | 316 | GGCTGTTG GGCTAGCTACAACGA AGGTGACC | 5113 |
| 1317 | CACCUACA A CAGCCCUA | 317 | TAGGGCTG GGCTAGCTACAACGA TGTAGGTG | 5114 |
| 1320 | CUACAACA G CCCUAGUG | 318 | CACTAGGG GGCTAGCTACAACGA TGTTGTAG | 5115 |
| 1326 | CAGCCCUA G UGGUAUCG | 319 | CGATACGA GGCTAGCTACAACGA TAGGGCTG | 5116 |
| 1329 | CCCUAGUG G UAUCGCAG | 320 | CTGCGATA GGCTAGCTACAACGA CACTAGGG | 5117 |
| 1331 | CUAGUGGU A UCGCAGUU | 321 | AACTGCGA GGCTAGCTACAACGA ACCACTAG | 5118 |
| 1334 | GUGGUAUC G CAGUUGCU | 322 | AGCAACTG GGCTAGCTACAACGA GATACCAC | 5119 |
| 1337 | GUAUCGCA G UUGCUCCG | 323 | CGGAGCAA GGCTAGCTACAACGA TGCGATAC | 5120 |
| 1340 | UCGCAGUU G CUCCGGAU | 324 | ATCCGGAG GGCTAGCTACAACGA AACTGCGA | 5121 |
| 1347 | UGCUCCGG A UCCCACAA | 325 | TTGTGGGA GGCTAGCTACAACGA CCGGAGCA | 5122 |
| 1352 | CGGAUCCC A CAAGCCGU | 326 | ACGGCTTG GGCTAGCTACAACGA GGGATCCG | 5123 |
| 1356 | UCCCACAA G CCGUCGUG | 327 | CACGACGG GGCTAGCTACAACGA TTGTGGGA | 5124 |
| 1359 | CACAAGCC G UCGUGGAC | 328 | GTCCACGA GGCTAGCTACAACGA GGCTTGTG | 5125 |
| 1362 | AAGCCGUC G UGGACAUG | 329 | CATGTCGA GGCTAGCTACAACGA GACGGCTT | 5126 |
| 1366 | CGUCGUGG A CAUGGUGG | 330 | CCACCATG GGCTAGCTACAACGA CCACGACG | 5127 |
| 1368 | UCCUGGAC A UGGUGGCG | 331 | CGCCACGA GGCTAGCTACAACGA GTCCACGA | 5128 |
| 1371 | UGGACAUG G UGGCGGGG | 332 | CCCCGCGA GGCTAGCTACAACGA CATGTCCA | 5129 |
| 1374 | ACAUGGUG G CCGGGGCC | 333 | CGCCCCCG GGCTAGCTACAACGA CACCATGT | 5130 |
| 1380 | UGGCGGGG G CCCACUGG | 334 | CCAGTGGG GGCTAGCTACAACGA CCCCGCCA | 5131 |
| 1384 | GCGGGCCC A CUGGGGAG | 335 | CTCCCCAC GGCTAGCTACAACGA GGGCCCCC | 5132 |
| 1392 | ACUGGGGA G UCCUGGCG | 336 | CGCCAGGA GGCTAGCTACAACGA TCCCCAGT | 5133 |
| 1398 | GAGUCCUG G CGGGCCUU | 337 | AAGGCCCG GGCTAGCTACAACGA CAGGACTC | 5134 |
| 1402 | CCUCGCGG G CCUUGCCU | 338 | AGCCAAGG GGCTAGCTACAACGA CCGCCAGG | 5135 |
| 1407 | CGGGCCUU G CCUAUUAU | 339 | ATAATAGG GGCTAGCTACAACGA AAGGCCCG | 5136 |
| 1411 | CCUUGCCU A UUAUUCGA | 340 | TGGAATAA GGCTAGCTACAACGA AGGCAAGG | 5137 |
| 1414 | UGCCUAUU A UUCCAUGG | 341 | CCATGGAA GGCTAGCTACAACGA AATAGGCA | 5138 |
| 1419 | AUUAUUCC A UGGUGGGG | 342 | CCCCACGA GGCTAGCTACAACGA GGAATAAT | 5139 |
| 1422 | AUUCCAUG G UGGGGAAC | 343 | GTTCCCGA GGCTAGCTACAACGA CATGGAAT | 5140 |
| 1429 | GGUGGGGA A CUGGGCUA | 344 | TAGCCCAG GGCTAGCTACAACGA TCCCCACC | 5141 |
| 1434 | GGAACUGG G CUAAGGUG | 345 | CACCTTAG GGCTAGCTACAACGA CCAGTTCC | 5142 |
| 1440 | GGGCUAAG G UGUUGAUU | 346 | AATCAACA GGCTAGCTACAACGA CTTAGCCC | 5143 |
| 1442 | GCUAAGGU G UUGAUUGU | 347 | ACAATCAA GGCTAGCTACAACGA ACCTTAGC | 5144 |
| 1446 | AGCUGUUG A UUGUCAUG | 348 | CATCACAA GGCTAGCTACAACGA CAACACCT | 5145 |
| 1449 | UGUUGAUU G UGAUGCUA | 349 | TAGCATCA GGCTAGCTACAACGA AATCAACA | 5146 |
| 1452 | UCAUUCUC A UGCUACUC | 350 | GAGTAGCA GGCTAGCTACAACGA CACAATCA | 5147 |
| 1454 | AUUGUCAU G CUACUCUU | 351 | AAGAGTAG GGCTAGCTACAACGA ATCACAAT | 5148 |
| 1457 | GUGAUCCU A CUCUUUGC | 352 | GCAAAGAG GGCTAGCTACAACGA AGCATCAC | 5149 |
| 1464 | UACUCUUU G CCGGCGUU | 353 | AACGCCGC GGCTAGCTACAACGA AAAGAGTA | 5150 |
| 1468 | CUUUGCCG G CCUUGACG | 354 | CGTCAACG GGCTAGCTACAACGA CCGCAPAG | 5151 |
| 1470 | UUGCCGGC G UUGACGGG | 355 | CCCGTCAA GGCTAGCTACAACGA GCCGGCAA | 5152 |
| 1474 | CGCCGUUC A CGGGGACA | 356 | TGTCCCCG GGCTAGCTACAACGA CAACGCCG | 5153 |
| 1480 | UGACGGGG A CACCUACA | 357 | TGTAGGTG GGCTAGCTACAACGA CCCCGTCA | 5154 |
| 1482 | ACGCCCAC A CCUACACG | 358 | CCTCTAGC GGCTAGCTACAACGA CTCCCCGT | 5155 |
| 1486 | GGACACCU A CACGACAG | 359 | CTCTCGTG GGCTAGCTACAACGA ACGTCTCC | 5156 |
| 1488 | ACACCUAC A CGACACCC | 360 | CCCTCTCC GGCTAGCTACAACGA GTAGGTCT | 5157 |
| 1491 | CCUACACG A CAGGCGGG | 361 | CCCCCCTG GGCTAGCTACAACGA CCTGTAGG | 5158 |
| 1500 | CAGCGGGG G CGCACCGC | 362 | CCCCTCCC GGCTAGCTACAACGA CCCCCCTC | 5159 |
| 1502 | CCGGCGGC G CACCCCGA | 363 | TCCCCCTC GGCTAGCTACAACGA GCCCCCCC | 5160 |
| 1507 | GGCGCACG G CCACACGA | 364 | TGGTGTGC GGCTAGCTACAACGA CCTGCGCC | 5161 |
| 1510 | GCACCCCC A CACCACUA | 365 | TAGTGGTC GGCTAGCTACAACGA CGCCCTCC | 5162 |
| 1512 | ACCCCCAC A CCACUACU | 366 | ACTAGTGC GGCTAGCTACAACGA GTGCCCCT | 5163 |
| 1515 | GCCACACC A CUAGUACG | 367 | CCTACTAG GGCTAGCTACAACGA CCTCTGGC | 5164 |
| 1519 | CACCACUA G UACCGUGG | 368 | CCACCCTA GGCTAGCTACAACGA TAGTCCTC | 5165 |
| 1524 | CUACUACC G UCCCAUCC | 369 | CCATGCGA GGCTAGCTACAACGA CCTACTAC | 5166 |
| 1527 | GUACCGUC G CAUCCCUC | 370 | GAGCCATC GGCTAGCTACAACGA CACCCTAC | 5167 |
| 1529 | ACCCUGGC A UCCCUCUU | 371 | AACACGCA GGCTAGCTACAACGA GCCACCCT | 5168 |
| 1539 | CCCUCUUU A CAUCUGGA | 372 | TCCACATG GGCTAGCTACAACGA AAAGACCC | 5169 |
| 1541 | CUCUUUAC A UCUGGAGC | 373 | GCTCCACA GGCTAGCTACAACGA GTAAAGAG | 5170 |
| 1548 | CAUCUCGA G CAUCUCAC | 374 | CTGAGATC GGCTAGCTACAACGA TCCACATG | 5171 |
| 1550 | UCUCGAGC A UCUCACAA | 375 | TTCTGACA GGCTAGCTACAACGA GCTCCAGA | 5172 |
| 1558 | AUCUCAGA A UAUCCACC | 376 | CCTCCATA GGCTAGCTACAACGA TCTGACAT | 5173 |
| 1560 | CUCAGAAU A UCCAGCUU | 377 | AACCTCGA GGCTAGCTACAACGA ATTCTGAG | 5174 |
| 1565 | AAUAUCGA G CUUAUUAA | 378 | TTAATAAC GGCTAGCTACAACGA TCCATATT | 5175 |
| 1569 | UCCAGCUU A UUAACACC | 379 | CGTGTTAA GGCTAGCTACAACGA AACCTCGA | 5176 |
| 1573 | CCUUAUUA A CACCAACC | 380 | CCTTCCTG GGCTAGCTACAACGA TAATAACC | 5177 |
| 1575 | UUAUUAAC A CCAACCCC | 381 | CCCCTTCC GGCTAGCTACAACGA GTTAATAA | 5178 |
| 1579 | UAACACCA A CCCCAGCU | 382 | ACCTGCCC GGCTAGCTACAACGA TCCTCTTA | 5179 |
| 1582 | CACCAACC G CACCUCCC | 383 | CCCACCTC GGCTAGCTACAACGA CCTTCCTC | 5180 |
| 1585 | CAACCCGA G CUCCCACA | 384 | TCTCCCAC GGCTAGCTACAACGA TCCCCTTC | 5181 |
| 1589 | CGCAGCUG G CACAUUAA | 385 | TTAATGTG GGCTAGCTACAACGA CAGCTCCC | 5182 |
| 1591 | CACCUCCC A CAUUAACA | 386 | TCTTAATC GGCTAGCTACAACGA CCCACCTC | 5183 |
| 1593 | CCUCCCAC A UUAACACC | 387 | CCTCTTAA GGCTAGCTACAACGA CTCCCACC | 5184 |
| 1597 | CCACAUUA A CACCACUG | 388 | CACTCCTC GGCTAGCTACAACGA TAATCTCC | 5185 |
| 1602 | UUAACACC A CUCCCCUC | 389 | CACCCCAC GGCTAGCTACAACGA CCTCTTAA | 5186 |
| 1605 | ACACCACU G CCCUCAAC | 390 | CTTCACCC GGCTAGCTACAACGA ACTCCTCT | 5187 |
| 1612 | UCCCCUCA A CUCCAAUC | 391 | CATTCCAC GGCTAGCTACAACGA TCAGCCCA | 5188 |
| 1615 | CCUCAACU G CAAUCACU | 392 | ACTCATTC GGCTAGCTACAACGA ACTTCACC | 5189 |
| 1618 | CAACUCCA A UCACUCCC | 393 | CCCACTCA GGCTAGCTACAACGA TGCAGTTC | 5190 |
| 1621 | CUCCAAUC A CUCCCUCC | 394 | CCACCCAC GGCTAGCTACAACGA CATTCCAC | 5191 |
| 1632 | CCCUCCAA A CCCCGUUC | 395 | CAACCCGG GGCTAGCTACAACGA TTGGAGCC | 5192 |
| 1637 | CAAACCGG G UUCAUUGC | 396 | GCAATGAA GGCTAGCTACAACGA CCGGTTTG | 5193 |
| 1641 | CCGGGUUC A UUGCUGCA | 397 | TGCAGCAA GGCTAGCTACAACGA GAACCCGG | 5194 |
| 1644 | GGUUCAUU G CUGCACUG | 398 | CAGTGCAG GGCTAGCTACAACGA AATGAACC | 5195 |
| 1647 | UCAUUGCU G CACUGUUC | 399 | GAACAGTG GGCTAGCTACAACGA AGCAATGA | 5196 |
| 1649 | AUUGCUGC A CUGUUCUA | 400 | TAGAACAG GGCTAGCTACAACGA GCAGCAAT | 5197 |
| 1652 | GCUGCACU G UUCUAUGC | 401 | GCATAGAA GGCTAGCTACAACGA AGTGCAGC | 5198 |
| 1657 | ACUGUUCU A UGCACACA | 402 | TGTGTGCA GGCTAGCTACAACGA AGAACAGT | 5199 |
| 1659 | UGUUCUAU G CACACAGG | 403 | CCTGTGTG GGCTAGCTACAACGA ATAGAACA | 5200 |
| 1661 | UUCUAUGC A CACAGGUU | 404 | AACCTGTG GGCTAGCTACAACGA GCATAGAA | 5201 |
| 1663 | CUAUGCAC A CAGGUUCA | 405 | TGAACCTG GGCTAGCTACAACGA GTGCATAG | 5202 |
| 1667 | GCACACAG G UUCAACUC | 406 | GAGTTGAA GGCTAGCTACAACGA CTGTGTGC | 5203 |
| 1672 | CAGGUUCA A CUCGUCCG | 407 | CGGACGAG GGCTAGCTACAACGA TGAACCTG | 5204 |
| 1676 | UUCAACUC G UCCGGAUG | 408 | CATCCGGA GGCTAGCTACAACGA GAGTTGAA | 5205 |
| 1682 | UCGUCCGG A UGCCCACA | 409 | TGTGGGCA GGCTAGCTACAACGA CCGGACGA | 5206 |
| 1684 | GUCCGGAU G CCCACAGC | 410 | GCTGTGGG GGCTAGCTACAACGA ATCCGGAC | 5207 |
| 1688 | GGAUGCCC A CAGCGCUU | 411 | AAGCGCTG GGCTAGCTACAACGA GGGCATCC | 5208 |
| 1691 | UGCCCACA G CGCUUGGC | 412 | GCCAAGCG GGCTAGCTACAACGA TGTGGGCA | 5209 |
| 1693 | CCCACAGC G CUUGGCCA | 413 | TGGCCAAG GGCTAGCTACAACGA GCTGTGGG | 5210 |
| 1698 | AGCGCUUG G CCAGCUGC | 414 | GCAGCTGG GGCTAGCTACAACGA CAAGCGCT | 5211 |
| 1702 | CUUGGCGA G CUGCCGCU | 415 | AGCGGCAG GGCTAGCTACAACGA TGGCCAAG | 5212 |
| 1705 | GGCCAGCU G CCGCUCGA | 416 | TGGAGCGG GGCTAGCTACAACGA AGCTGGCC | 5213 |
| 1708 | CAGCUGCC G CUCCAUUG | 417 | CAATGGAG GGCTAGCTACAACGA GGCAGCTG | 5214 |
| 1713 | GCCGCUCC A UUGACAAG | 418 | CTTCTCAA GGCTAGCTACAACGA GGAGCGGC | 5215 |
| 1717 | CUCCAUUG A CAAGUUCG | 419 | CGAACTTC GGCTAGCTACAACGA CAATGGAG | 5216 |
| 1721 | AUUGACAA G UUCGCUCA | 420 | TCAGCGAA GGCTAGCTACAACGA TTGTCAAT | 5217 |
| 1725 | ACAAGUUC G CUCAGGGG | 421 | CCCCTGAG GGCTAGCTACAACGA GAACTTGT | 5218 |
| 1733 | GCUCAGGG G UGGGGUCC | 422 | GGACCCGA GGCTAGCTACAACGA CCCTGAGC | 5219 |
| 1738 | GGGGUGGG G UCCUAUCA | 423 | TGATAGGA GGCTAGCTACAACGA CCCACCCC | 5220 |
| 1743 | GGGGUCCU A UCACCUAC | 424 | GTAGGTGA GGCTAGCTACAACGA AGGACCCC | 5221 |
| 1746 | GUCCUAUC A CCUACACC | 425 | GGTGTAGG GGCTAGCTACAACGA GATAGGAC | 5222 |
| 1750 | UAUCACCU A CACCGAGG | 426 | CCTCGGTG GGCTAGCTACAACGA AGGTGATA | 5223 |
| 1752 | UCACCUAC A CCGAGGGC | 427 | GCCCTCGG GGCTAGCTACAACGA GTAGGTGA | 5224 |
| 1759 | CACCGAGG G CCACAACU | 428 | ACTTGTGG GGCTAGCTACAACGA CCTCGGTG | 5225 |
| 1762 | CGAGGGCC A CAACUCGG | 429 | CCGAGTTG GGCTAGCTACAACGA GGCCCTCG | 5226 |
| 1765 | GGGCCACA A CUCGGACC | 430 | GGTCCGAG GGCTAGCTACAACGA TGTGGCCC | 5227 |
| 1771 | CAACUCGG A CCAGAGGC | 431 | GCCTCTGG GGCTAGCTACAACGA CCGAGTTG | 5228 |
| 1778 | GACCAGAG G CCCUAUUG | 432 | CAATAGGG GGCTAGCTACAACGA CTCTGGTC | 5229 |
| 1783 | GAGGCCCU A UUGCUGGC | 433 | GCCAGCAA GGCTAGCTACAACGA AGGGCCTC | 5230 |
| 1786 | GCCCUAUU G CUGGCACU | 434 | AGTGCCAG GGCTAGCTACAACGA AATAGGGC | 5231 |
| 1790 | UAUUGCUG G CACUACGC | 435 | GCGTAGTG GGCTAGCTACAACGA CAGCAATA | 5232 |
| 1792 | UUGCUGGC A CUACGCAC | 436 | GTGCGTAG GGCTAGCTACAACGA GCCAGCAA | 5233 |
| 1795 | CUGGCACU A CGCACCGC | 437 | GCGGTGCG GGCTAGCTACAACGA AGTGCCAG | 5234 |
| 1797 | GGCACUAC G CACCGCGG | 438 | CCGCGGTG GGCTAGCTACAACGA GTAGTGCC | 5235 |
| 1799 | CACUACGC A CCGCGGCC | 439 | GGCCGCGG GGCTAGCTACAACGA GCGTAGTG | 5236 |
| 1802 | UACGCACC G CGGCCGUG | 440 | CACGGCCG GGCTAGCTACAACGA GGTGCGTA | 5237 |
| 1805 | GCACCGCG G CCGUGUGG | 441 | CCACACGG GGCTAGCTACAACGA CGCGGTGC | 5238 |
| 1808 | CCGCGGCC G UGUGGUAU | 442 | ATACCACA GGCTAGCTACAACGA GGCCGCGG | 5239 |
| 1810 | GCGGCCGU G UGGUAUCG | 443 | CGATACGA GGCTAGCTACAACGA ACGGCCGC | 5240 |
| 1813 | GCCGUGUG G UAUCGUAC | 444 | GTACGATA GGCTAGCTACAACGA CACACGGC | 5241 |
| 1815 | CGUGUGGU A UCGUACCC | 445 | GGGTACGA GGCTAGCTACAACGA ACCACACG | 5242 |
| 1818 | GUGGUAUC G UACCCGCA | 446 | TGCGGGTA GGCTAGCTACAACGA GATACCAC | 5243 |
| 1820 | GGUAUCGU A CCCGCAUC | 447 | GATGCGGG GGCTAGCTACAACGA ACGATACCC | 5244 |
| 1824 | UCGUACCC G CAUCGCAG | 448 | CTGCGATG GGCTAGCTACAACGA GGGTACGA | 5245 |
| 1826 | GUACCCGC A UCGCAGGU | 449 | ACCTGCGA GGCTAGCTACAACGA GCCGGTAC | 5246 |
| 1829 | CCCGCAUC G CAGGUAUG | 450 | CATACCTG GGCTAGCTACAACGA GATGCGGG | 5247 |
| 1833 | CAUCGCAG G UAUGUGGU | 451 | ACCACATA GGCTAGCTACAACGA CTGCGATG | 5248 |
| 1835 | UCGCAGGU A UGUGGUCC | 452 | GGACCACA GGCTAGCTACAACGA ACCTGCGA | 5249 |
| 1837 | GCAGGUAU G UGGUCCAG | 453 | CTGGACGA GGCTAGCTACAACGA ATACCTGC | 5250 |
| 1840 | GGUAUGUG G UCCAGUGU | 454 | ACACTGGA GGCTAGCTACAACGA CACATACC | 5251 |
| 1845 | GUGGUCGA G UGUAUUGC | 455 | GCAATACA GGCTAGCTACAACGA TGGACCAC | 5252 |
| 1847 | GGUCCAGU G UAUUGCUU | 456 | AAGCAATA GGCTAGCTACAACGA ACTGGACC | 5253 |
| 1849 | UCCAGUGU A UUGCUUCA | 457 | TGAAGCAA GGCTAGCTACAACGA ACACTGGA | 5254 |
| 1852 | AGUGUAUU G CUUCACCC | 458 | GGGTGAAG GGCTAGCTACAACGA AATACACT | 5255 |
| 1857 | AUUGCUUC A CCCCAAGC | 459 | GCTTGGGG GGCTAGCTACAACGA GAAGCAAT | 5256 |
| 1864 | CACCCCAA G CCCUGGUG | 460 | CAACAGGG GGCTAGCTACAACGA TTGGGGTG | 5257 |
| 1869 | CAAGCCCU G UUGUGGUG | 461 | CACCACAA GGCTAGCTACAACGA AGGGCTTG | 5258 |
| 1872 | GCCCUGUU G UGGUGGGG | 462 | CCCCACGA GGCTAGCTACAACGA AACAGGGC | 5259 |
| 1875 | CUGGUGUG G UGGGGACG | 463 | CGTCCCGA GGCTAGCTACAACGA CACAACAG | 5260 |
| 1881 | UGGUGGGG A CGACCGAC | 464 | GTCGGTCG GGCTAGCTACAACGA CCCCACCA | 5261 |
| 1884 | UGGGGACG A CCGACCGU | 465 | ACGGTCGG GGCTAGCTACAACGA CGTCCCCA | 5262 |
| 1888 | GACGACCG A CCGUUUCG | 466 | CGAAACGG GGCTAGCTACAACGA CGGTCGTC | 5263 |
| 1891 | GACCGACC G UUUCGGCG | 467 | CGCCGAAA GGCTAGCTACAACGA GGTCGGTC | 5264 |
| 1897 | CCGUUUCG G CGCCCCCA | 468 | TGGGGGCG GGCTAGCTACAACGA CGAAACGG | 5265 |
| 1899 | GUUUCGGC G CCCCCACG | 469 | CGTGGGGG GGCTAGCTACAACGA GCCGAAAC | 5266 |
| 1905 | GCGCCCCC A CGUAUAAC | 470 | GTTATACG GGCTAGCTACAACGA GGGGGCGC | 5267 |
| 1907 | GCCCCCAC G UAUAACUG | 471 | CAGTTATA GGCTAGCTACAACGA GTGGGGGC | 5268 |
| 1909 | CCCCACGU A UAACUGGG | 472 | CCCAGTTA GGCTAGCTACAACGA ACGTGGGG | 5269 |
| 1912 | CACGUAUA A CUGCGGGG | 473 | CCCCCCAG GGCTAGCTACAACGA TATACGTG | 5270 |
| 1920 | ACUGGGGG G CGAACGAG | 474 | CTCGTTCG GGCTAGCTACAACGA CCCCCAGT | 5271 |
| 1924 | GGGGGCCA A CGAGACGG | 475 | CCGTCTCG GGCTAGCTACAACGA TCGCCCCC | 5272 |
| 1929 | CGAACGAG A CGGACGUG | 476 | CACGTCCG GGCTAGCTACAACGA CTCGTTCG | 5273 |
| 1933 | CGAGACGG A CGUGCUGC | 477 | GCAGCACG GGCTAGCTACAACGA CCGTCTCG | 5274 |
| 1935 | AGACGGAC G UGCUGCUC | 478 | GACCACGA GGCTAGCTACAACGA GTCCGTCT | 5275 |
| 1937 | ACGGACGU G CUCCUCCU | 479 | ACGAGCAG GGCTAGCTACAACGA ACGTCCCT | 5276 |
| 1940 | CACGUGCU G CUCCUCAA | 480 | TTGAGGAC GGCTAGCTACAACGA ACCACCTC | 5277 |
| 1948 | GCUCCUCA A CAACACGC | 481 | GCGTGTTG GGCTAGCTACAACGA TGACCACC | 5278 |
| 1951 | CCUCAACA A CACGCGGC | 482 | CCCCCGTC GGCTAGCTACAACGA TGTTGAGG | 5279 |
| 1953 | UCAACAAC A CCCCGCCC | 483 | CGGCCGCG GGCTAGCTACAACGA CTTGTTCA | 5280 |
| 1955 | AACAACAC G CCCCCGCC | 484 | GCCGGCCC GGCTAGCTACAACGA GTCTTCTT | 5281 |
| 1958 | AACACCCG G CCCCCCGA | 485 | TGCGGCCC GGCTAGCTACAACGA CGCGTGTT | 5282 |
| 1961 | ACGCCCCC G CCGCAAGG | 486 | CCTTCCCC GGCTAGCTACAACGA CGCCCCCT | 5283 |
| 1964 | CGGCCGCC G CAAGGCAA | 487 | TTCCCTTC GGCTAGCTACAACGA CGCCCCCC | 5284 |
| 1969 | GCCGCAAC G CAACUGGU | 488 | ACCACTTC GGCTAGCTACAACGA CTTCCCCC | 5285 |
| 1972 | GCAAGCCA A CUGGUUCG | 489 | CCAACCAG GGCTAGCTACAACGA TCCCTTCC | 5286 |
| 1976 | GGCAACUG G UUCGGCUG | 490 | CAGCCGAA GGCTAGCTACAACGA CACTTCCC | 5287 |
| 1981 | CUGGUUCG G CUCCACAU | 491 | ATGTGCAG GGCTAGCTACAACGA CGAACCAG | 5288 |
| 1984 | GUUCGGCU G CACAUCCA | 492 | TCCATCTC GGCTAGCTACAACGA AGCCGAAC | 5289 |
| 1986 | UCCCCUCC A CAUCCAUC | 493 | CATCCATC GGCTAGCTACAACGA CCACCCCA | 5290 |
| 1988 | CCCUCCAC A UGGAUGAA | 494 | TTCATCGA GGCTAGCTACAACGA CTCCACCC | 5291 |
| 1992 | GCACAUCG A UGAAUGGC | 495 | CCCATTCA GGCTAGCTACAACGA CCATCTCC | 5292 |
| 1996 | AUCCAUCA A UCCCACUC | 496 | CACTCCGA GGCTAGCTACAACGA TCATCCAT | 5293 |
| 1999 | CAUCAAUC G CACUGCCU | 497 | ACCCACTC GGCTAGCTACAACGA CATTCATC | 5294 |
| 2001 | UCAAUCCC A CUCCCUUC | 498 | GAACCCAC GGCTAGCTACAACGA CCCATTCA | 5295 |
| 2006 | CCCACUCC G UUCACCAA | 499 | TTCCTCAA GGCTAGCTACAACGA CCACTCCC | 5296 |
| 2010 | CUCCCUUC A CCAACACC | 500 | CCTCTTCC GGCTAGCTACAACGA CAACCCAC | 5297 |
| 2016 | UCACCAAG A CCUCCGGG | 501 | CCCCCACC GGCTAGCTACAACGA CTTCCTCA | 5298 |
| 2018 | ACCAACAC G UGCGGGGG | 502 | CCCCCCGA GGCTAGCTACAACGA CTCTTCCT | 5299 |
| 2020 | CAACACGU G CCGGGGCC | 503 | CGCCCCCG GGCTAGCTACAACGA ACCTCTTG | 5300 |
| 2026 | CUCCCCCC G CCCCCCGU | 504 | ACCGCCCG GGCTAGCTACAACGA CCCCCCAC | 5301 |
| 2033 | CGCCCCCC G UCCAACAU | 505 | ATCTTCGA GGCTAGCTACAACGA GGGGGGCC | 5302 |
| 2035 | CCCCCCGU G CAACAUCG | 506 | CCATCTTC GGCTAGCTACAACGA ACGCCCCC | 5303 |
| 2038 | CCCCUGCA A CAUGGGGG | 507 | CCCCCATC GGCTAGCTACAACGA TCCACCCC | 5304 |
| 2040 | CGUGCAAC A UCGGGGGG | 508 | CCCCCCGA GGCTAGCTACAACGA CTTCCACC | 5305 |
| 2049 | UCGGGGGG G CCGGUAAC | 509 | CTTACCCC GGCTAGCTACAACGA CCCCCCGA | 5306 |
| 2053 | GGGGGCCG G UAACGACA | 510 | TGTCGTTA GGCTAGCTACAACGA CGGCCCCC | 5307 |
| 2058 | GGCCGGUA A CGACACCU | 511 | AGGTGTCG GGCTAGCTACAACGA TACCGGCC | 5308 |
| 2059 | CGGUAACG A CACCUUAA | 512 | TTAAGGTG GGCTAGCTACAACGA CGTTACCG | 5309 |
| 2061 | GUAACGAC A CCUUAACC | 513 | GGTTAAGG GGCTAGCTACAACGA GTCGTTAC | 5310 |
| 2067 | ACACCUUA A CCUGCCCC | 514 | GGGGCAGG GGCTAGCTACAACGA TAAGGTGT | 5311 |
| 2071 | CUUAACCU G CCCCACGG | 515 | CCGTGGGG GGCTAGCTACAACGA AGGTTAAG | 5312 |
| 2076 | CCUGCCCC A CGGACUGC | 516 | GCAGTCCG GGCTAGCTACAACGA GGGGCAGG | 5313 |
| 2080 | CCCCACGG A CUGCUUCC | 517 | GGAAGCAG GGCTAGCTACAACGA CCGTGGGG | 5314 |
| 2083 | CACGGACU G CUUCCGGA | 518 | TCCGGAAG GGCTAGCTACAACGA AGTCCCCG | 5315 |
| 2093 | UUCCGGAA G CACCCCGA | 519 | TCGGGGTG GGCTAGCTACAACGA TTCCGGAA | 5316 |
| 2095 | CCGGAAGC A CCCCGAGG | 520 | CCTCGGGG GGCTAGCTACAACGA GCTTCCGG | 5317 |
| 2103 | ACCCCGAG G CCACUUAC | 521 | GTAAGTGG GGCTAGCTACAACGA CTCGGGGT | 5318 |
| 2106 | CCGAGGCC A CUUACGCA | 522 | TGCGTAAG GGCTAGCTACAACGA GGCCTCGG | 5319 |
| 2110 | GGCCACUU A CGCAAAGU | 523 | ACTTTGCG GGCTAGCTACAACGA AAGTGGCC | 5320 |
| 2112 | CCACUUAC G CAAAGUGC | 524 | GCACTTTG GGCTAGCTACAACGA GTAAGTGG | 5321 |
| 2117 | UACGCAAA G UGCGGUUC | 525 | GAACCGCA GGCTAGCTACAACGA TTTGCGTA | 5322 |
| 2119 | CGCAAAGU G CGGUUCGG | 526 | CCGAACCG GGCTAGCTACAACGA ACTTTGCG | 5323 |
| 2122 | AAAGUGCG G UUCGGGGC | 527 | GCCCCGAA GGCTAGCTACAACGA CGCACTTT | 5324 |
| 2129 | GGUUCGGG G CCUUGGUU | 528 | AACCAAGG GGCTAGCTACAACGA CCCGAACC | 5325 |
| 2135 | CGGCCUUG G UUAACACC | 529 | GGTGTTAA GGCTAGCTACAACGA CAAGGCCC | 5326 |
| 2139 | CUUGGUUA A CACCUAGA | 530 | TCTAGGTG GGCTAGCTACAACGA TAACCAAG | 5327 |
| 2141 | UGGUUAAC A CCUAGAUG | 531 | CATCTAGG GGCTAGCTACAACGA GTTAACCA | 5328 |
| 2147 | ACACCUAG A UGCAUAGU | 532 | ACTATGCA GGCTAGCTACAACGA CTAGGTGT | 5329 |
| 2149 | ACCUAGAU G CAUAGUUG | 533 | CAACTATG GGCTAGCTACAACGA ATCTAGGT | 5330 |
| 2151 | CUAGAUGC A UAGUUGAC | 534 | GTCAACTA GGCTAGCTACAACGA GCATCTAG | 5331 |
| 2154 | GAUGCAUA G UUGACUAC | 535 | GTAGTCAA GGCTAGCTACAACGA TATGCATC | 5332 |
| 2158 | CAUAGUUG A CUACCCAU | 536 | ATGGGTAG GGCTAGCTACAACGA CAACTATG | 5333 |
| 2161 | AGUUGACU A CCCAUACA | 537 | TGTATGGG GGCTAGCTACAACGA AGTCAACT | 5334 |
| 2165 | GACUACCC A UACAGGCU | 538 | AGCCTGTA GGCTAGCTACAACGA GGGTAGTC | 5335 |
| 2167 | CUACCCAU A CAGGCUUU | 539 | AAAGCCTG GGCTAGCTACAACGA ATGGGTAG | 5336 |
| 2171 | CCAUACAG G CUUUGGCA | 540 | TGCCAAAG GGCTAGCTACAACGA CTGTATGG | 5337 |
| 2177 | AGGCUUUG G CACUACCC | 541 | GGGTAGTG GGCTAGCTACAACGA CAAAGCCT | 5338 |
| 2179 | GCUUUGGC A CUACCCCU | 542 | AGGGGTAG GGCTAGCTACAACGA GCCAAAGC | 5339 |
| 2182 | UUGGCACU A CCCCUGCA | 543 | TGCAGGGG GGCTAGCTACAACGA AGTGCCAA | 5340 |
| 2188 | CUACCCCU G CACUGUCA | 544 | TGACAGTG GGCTAGCTACAACGA AGGGGTAG | 5341 |
| 2190 | ACCCCUGC A CUGUCAAU | 545 | ATTGACAG GGCTAGCTACAACGA GCAGGGGT | 5342 |
| 2193 | CCUGCACU G UCAAUUUU | 546 | AAAATTGA GGCTAGCTACAACGA AGTGCAGG | 5343 |
| 2197 | CACUGUCA A UUUUUCCA | 547 | TGGAAAAA GGCTAGCTACAACGA TGACAGTG | 5344 |
| 2205 | AUUUUUCC A UCUUUAAG | 548 | CTTAAAGA GGCTAGCTACAACGA GGAAAAAT | 5345 |
| 2214 | UCUUUAAG G UUAGGAUG | 549 | CATCCTAA GGCTAGCTACAACGA CTTAAAGA | 5346 |
| 2220 | AGGUUAGG A UGUAUGUG | 550 | CACATACA GGCTAGCTACAACGA CCTAACCT | 5347 |
| 2222 | GUUAGGAU G UAUGUGGG | 551 | CCCACATA GGCTAGCTACAACGA ATCCTAAC | 5348 |
| 2224 | UAGGAUGU A UGUGGGGG | 552 | CCCCCACA GGCTAGCTACAACGA ACATCCTA | 5349 |
| 2226 | GGAUGUAU G UGGGGGGC | 553 | GCCCCCGA GGCTAGCTACAACGA ATACATCC | 5350 |
| 2233 | UGUGGGGG G CGUGGAGC | 554 | GGCTCCACG GGCTAGCTACAACGA CCCCCACA | 5351 |
| 2235 | UGGGGGGC G UGGACCAC | 555 | GTGCTCGA GGCTAGCTACAACGA GCCCCCCA | 5352 |
| 2240 | GGCGUGGA G CACAGGCU | 556 | AGCCTGTG GGCTAGCTACAACGA TCCACGCC | 5353 |
| 2242 | CGUGGAGC A CAGGCUCA | 557 | TGAGCCTG GGCTAGCTACAACGA GCTCCACG | 5354 |
| 2246 | GAGCACAG G CUCACCGC | 558 | GCGGTGAG GGCTAGCTACAACGA CTGTGCTC | 5355 |
| 2250 | ACAGGCUC A CCGCCGCA | 559 | TGCGGCGG GGCTAGCTACAACGA GAGCCTGT | 5356 |
| 2253 | GGCUCACC G CCGCAUGC | 560 | GCATGCGG GGCTAGCTACAACGA GGTGAGCC | 5357 |
| 2256 | UCACCGCC G CAUGCAAU | 561 | ATTGCATG GGCTAGCTACAACGA GGCGGTGA | 5358 |
| 2258 | ACCGCCGC A UGCAAUUG | 562 | CAATTGCA GGCTAGCTACAACGA GCGGCGGT | 5359 |
| 2260 | CGCCGCAU G CAAUUGGA | 563 | TCCAATTG GGCTAGCTACAACGA ATGCGGCG | 5360 |
| 2263 | CGCAUGCA A UUGGACUC | 564 | GAGTCCAA GGCTAGCTACAACGA TGCATGCG | 5361 |
| 2268 | GCAAUUGG A CUCGAGGA | 565 | TCCTCGAG GGCTAGCTACAACGA CCAATTGC | 5362 |
| 2279 | CGAGGAGA G CGUUGUGA | 566 | TCACAACG GGCTAGCTACAACGA TCTCCTCG | 5363 |
| 2281 | AGGAGAGC G UUGUGAUU | 567 | AATCACAA GGCTAGCTACAACGA GCTCTCCT | 5364 |
| 2284 | AGAGCGUU G UGAUUUGG | 568 | CCAAATCA GGCTAGCTACAACGA AACGCTCT | 5365 |
| 2287 | GCGUUGUG A UUUGGAGG | 569 | CCTCCAAA GGCTAGCTACAACGA CACAACGC | 5366 |
| 2296 | UUUGGAGG A CAGGGACA | 570 | TGTCCCTG GGCTAGCTACAACGA CCTCCAAA | 5367 |
| 2302 | GGACAGGG A CAGAUCAG | 571 | CTGATCTG GGCTAGCTACAACGA CCCTGTCC | 5368 |
| 2306 | AGGGACAG A UCAGAGCU | 572 | AGCTCTGA GGCTAGCTACAACGA CTGTCCCT | 5369 |
| 2312 | AGAUCAGA G CUCAGCCC | 573 | GGGCTGAG GGCTAGCTACAACGA TCTGATCT | 5370 |
| 2317 | AGAGCUCA G CCCGCUGC | 574 | GCAGCGGG GGCTAGCTACAACGA TGAGCTCT | 5371 |
| 2321 | CUCAGCCC G CUGCUGUU | 575 | AACAGCAG GGCTAGCTACAACGA GGGCTGAG | 5372 |
| 2324 | AGCCCGCU G CUGUUGUC | 576 | GACAACAG GGCTAGCTACAACGA AGCGGGCT | 5373 |
| 2327 | CCGCUGCU G UUGUCCAC | 577 | GTGGACAA GGCTAGCTACAACGA AGCAGCGG | 5374 |
| 2330 | CUGCUGUU G UCCACUAC | 578 | GTAGTGGA GGCTAGCTACAACGA AACAGCAG | 5375 |
| 2334 | UGUUGUCC A CUACAGAG | 579 | CTCTGTAG GGCTAGCTACAACGA GGACAACA | 5376 |
| 2337 | UGUCCACU A CAGAGUGG | 580 | CCACTCTG GGCTAGCTACAACGA AGTGGACA | 5377 |
| 2342 | ACUACAGA G UGGCAAAU | 581 | ATTTGCGA GGCTAGCTACAACGA TCTGTAGT | 5378 |
| 2345 | ACAGAGUG G CAAAUACU | 582 | AGTATTTG GGCTAGCTACAACGA CACTCTGT | 5379 |
| 2349 | AGUGGCAA A UACUGCCC | 583 | GGGCAGTA GGCTAGCTACAACGA TTGCCACT | 5380 |
| 2351 | UGGCAAAU A CUGCCCUG | 584 | CAGGGCAG GGCTAGCTACAACGA ATTTGCCA | 5381 |
| 2354 | CAAAUACU G CCCUGCUC | 585 | GAGCAGGG GGCTAGCTACAACGA AGTATTTG | 5382 |
| 2359 | ACUGCCCU G CUCCUUCA | 586 | TGAAGGAG GGCTAGCTACAACGA AGGGCAGT | 5383 |
| 2367 | GCUCCUUC A CCACCCUA | 587 | TAGGGTGG GGCTAGCTACAACGA GAAGGAGC | 5384 |
| 2370 | CCUUCACC A CCCUACCG | 588 | CGGTAGGG GGCTAGCTACAACGA GGTGAAGG | 5385 |
| 2375 | ACCACCCU A CCGGCUCU | 589 | AGAGCCGG GGCTAGCTACAACGA AGGGTGGT | 5386 |
| 2379 | CCCUACCG G CUCUGUCC | 590 | GGACAGAG GGCTAGCTACAACGA CGGTAGGG | 5387 |
| 2384 | CCGGCUCU G UCCACUGG | 591 | CCAGTGGA GGCTAGCTACAACGA AGAGCCGG | 5388 |
| 2388 | CUCUGUCC A CUGGUUUG | 592 | CAAACCAG GGCTAGCTACAACGA GGACAGAG | 5389 |
| 2392 | GUCCACUG G UUUGAUCC | 593 | GGATCAAA GGCTAGCTACAACGA CAGTGGAC | 5390 |
| 2397 | CUGGUUUG A UCCAUCUC | 594 | GAGATGGA GGCTAGCTACAACGA CAAACCAG | 5391 |
| 2401 | UUUGAUCC A UCUCCACC | 595 | GGTGGAGA GGCTAGCTACAACGA GGATCAAA | 5392 |
| 2407 | CCAUCUCC A CCAGAACA | 596 | TGTTCTGG GGCTAGCTACAACGA GGAGATGG | 5393 |
| 2413 | CCACCAGA A CAUCGUGG | 597 | CCACGATG GGCTAGCTACAACGA TCTGGTGG | 5394 |
| 2415 | ACCAGAAC A UCGUGGAC | 598 | GTCCACGA GGCTAGCTACAACGA GTTCTGGT | 5395 |
| 2418 | AGAACAUC G UGGACCUG | 599 | CACGTCGA GGCTAGCTACAACGA GATGTTCT | 5396 |
| 2422 | CAUCGUGG A CGUGCAAU | 600 | ATTGCACG GGCTAGCTACAACGA CCACGATG | 5397 |
| 2424 | UCGUGGAC G UGCAAUAC | 601 | GTATTGCA GGCTAGCTACAACGA GTCCACGA | 5398 |
| 2426 | GUGGACGU G CAAUACCU | 602 | AGGTATTG GGCTAGCTACAACGA ACGTCCAC | 5399 |
| 2429 | GACGUGCA A UACCUGUA | 603 | TACAGGTA GGCTAGCTACAACGA TGCACGTC | 5400 |
| 2431 | CGUGCAAU A CCUGUACG | 604 | CGTACAGG GGCTAGCTACAACGA ATTGCACG | 5401 |
| 2435 | CAAUACCU G UACGGUGU | 605 | ACACCGTA GGCTAGCTACAACGA AGGTATTG | 5402 |
| 2437 | AUACCUGU A CGGUGUAG | 606 | CTACACCG GGCTAGCTACAACGA ACAGGTAT | 5403 |
| 2440 | CCUGUACG G UGUAGGGU | 607 | ACCCTACA GGCTAGCTACAACGA CGTACAGG | 5404 |
| 2442 | UGUACGGU G UAGGGUCA | 608 | TGACCCTA GGCTAGCTACAACGA ACCGTACA | 5405 |
| 2447 | GGUGUAGG G UCAGCGGU | 609 | ACCGCTGA GGCTAGCTACAACGA CCTACACC | 5406 |
| 2451 | UAGGGUCA G CGGUUGUC | 610 | GACAACCG GGCTAGCTACAACGA TGACCCTA | 5407 |
| 2454 | GGUCAGCG G UUGUCUCC | 611 | GGAGACAA GGCTAGCTACAACGA CGCTGACC | 5408 |
| 2457 | CAGCGGUU G UCUCCUUC | 612 | GAAGGAGA GGCTAGCTACAACGA AACCGCTG | 5409 |
| 2466 | UCUCCUUC G CAAUCAAA | 613 | TTTGATTG GGCTAGCTACAACGA GAAGGAGA | 5410 |
| 2469 | CCUUCGCA A UCAAAUGG | 614 | CCATTTGA GGCTAGCTACAACGA TGCGAAGG | 5411 |
| 2474 | GCAAUCAA A UGGGAGUA | 615 | TACTCCGA GGCTAGCTACAACGA TTGATTGC | 5412 |
| 2480 | AAAUGGGA G UAUGUCCU | 616 | AGGACATA GGCTAGCTACAACGA TCCCATTT | 5413 |
| 2482 | AUGGGAGU A UGUCCUGU | 617 | ACAGGACA GGCTAGCTACAACGA ACTCCCAT | 5414 |
| 2484 | GGGAGUAU G UCCUGUUG | 618 | CAACAGGA GGCTAGCTACAACGA ATACTCCC | 5415 |
| 2489 | UAUGUCCU G UUGCUUUU | 619 | AAAAGCAA GGCTAGCTACAACGA AGGACATA | 5416 |
| 2492 | GUCCUGUU G CUUUUCCU | 620 | AGGAAAAG GGCTAGCTACAACGA AACAGGAC | 5417 |
| 2508 | UUCUCCUG G CAGACGCG | 621 | CGCGTCTG GGCTAGCTACAACGA CAGGAGAA | 5418 |
| 2512 | CCUGGCAG A CGCGCGCG | 622 | CGCGCGCG GGCTAGCTACAACGA CTGCCAGG | 5419 |
| 2514 | UGGCAGAC G CGCGCGUC | 623 | GACGCGCG GGCTAGCTACAACGA GTCTGCCA | 5420 |
| 2516 | GCAGACGC G CGCGUCUG | 624C | AGACGCG GGCTAGCTACAACGA GCGTCTGC | 5421 |
| 2518 | AGACGCGC G CGUCUGUG | 625 | CACAGACG GGCTAGCTACAACGA GCGCGTCT | 5422 |
| 2520 | ACGCGCGC G UCUGUGCC | 626 | GGCACAGA GGCTAGCTACAACGA GCGCGCGT | 5423 |
| 2524 | GCGCGUCU G UGCCUGUU | 627 | AACAGGCA GGCTAGCTACAACGA AGACGCGC | 5424 |
| 2526 | GCGUCUGU G CCUGUUUG | 628 | CAAACAGG GGCTAGCTACAACGA ACAGACGC | 5425 |
| 2530 | CUGUGCCU G UUUGUGGA | 629 | TCCACAAA GGCTAGCTACAACGA AGGCACAG | 5426 |
| 2534 | GCCUGUUU G UGGAUGAU | 630 | ATCATCGA GGCTAGCTACAACGA AAACAGGC | 5427 |
| 2538 | GUUUGUGG A UGAUGCUG | 631 | CAGCATCA GGCTAGCTACAACGA CCACAAAC | 5428 |
| 2541 | UGUGGAUG A UGCUGUUG | 632 | CAACAGCA GGCTAGCTACAACGA CATCCACA | 5429 |
| 2543 | UGGAUGAU G CUGUUGGU | 633 | ACCAACAG GGCTAGCTACAACGA ATCATCCA | 5430 |
| 2546 | AUGAUGCU G UUGGUAGC | 634 | GCTACCAA GGCTAGCTACAACGA AGCATCAT | 5431 |
| 2550 | UGCUGUUG G UAGCCCAG | 635 | CTGGGCTA GGCTAGCTACAACGA CAACAGCA | 5432 |
| 2553 | UGUUGGUA G CCCAGGCC | 636 | GGCCTGGG GGCTAGCTACAACGA TACCAACA | 5433 |
| 2559 | UAGCCCAG G CCGAGGCU | 637 | AGCCTCGG GGCTAGCTACAACGA CTGGGCTA | 5434 |
| 2565 | AGGCCGAG G CUGCCCUA | 638 | TAGGGCAG GGCTAGCTACAACGA CTCGGCCT | 5435 |
| 2568 | CCGAGGCU G CCCUAGAG | 639 | CTCTAGGG GGCTAGCTAGAACGA AGCCTCGG | 5436 |
| 2578 | CCUAGAGA A CCUGGUGG | 640 | CCACCAGG GGCTAGCTACAACGA TCTCTAGG | 5437 |
| 2583 | AGAACCUG G UGGUCCUC | 641 | GAGGACGA GGCTAGCTACAACGA CAGGTTCT | 5438 |
| 2586 | ACCUGGUG G UCCUCAAU | 642 | ATTGAGGA GGCTAGCTACAACGA CACCAGGT | 5439 |
| 2593 | GGUCCUCA A UGCAGCAU | 643 | ATGCTGCA GGCTAGCTACAACGA TGAGGACC | 5440 |
| 2595 | UCCUCAAU G CAGCAUCC | 644 | GGATGCTG GGCTAGCTACAACGA ATTGAGGA | 5441 |
| 2598 | UCAAUGCA G CAUCCUUG | 645 | CAAGGATG GGCTAGCTACAACGA TGCATTGA | 5442 |
| 2600 | AAUGCAGC A UCCUUGGC | 646 | GCCAAGGA GGCTAGCTACAACGA GCTGCATT | 5443 |
| 2607 | CAUCCUUG G CCGGAGUG | 647 | CACTCCGG GGCTAGCTACAACGA CAAGGATG | 5444 |
| 2613 | UGGCCGGA G UGCAUGGC | 648 | GCCATUCA GGCTAUCTACAACGA TCCUGCCA | 5445 |
| 2615 | UCCGUAGU G CAUUGCAU | 649 | ATUCCATG GGCTAUCTACAACGA ACTCCGUC | 5446 |
| 2617 | CGGAGUGC A UUGCAUCC | 650 | GGATGCGA UGCTAGCTACAACGA GCACTCCG | 5447 |
| 2620 | AGUGCAUG G CAUCCUCU | 651 | AGAGGATG GGCTAGCTACAACGA CATGCACT | 5448 |
| 2622 | UGCAUGGC A UCCUCUCC | 652 | GGAGAGGA GGCTAUCTACAACGA GCCATGCA | 5449 |
| 2637 | CCUUCCUC G UGUUCUUC | 653 | GAAGAACA GGCTAGCTACAACGA GAGGAAGG | 5450 |
| 2639 | UUCCUCGU G UUCUUCUG | 654 | CAGAAGAA GGCTAGCTACAACGA ACGAGGAA | 5451 |
| 2647 | GUUCUUCU G UGCUGCCU | 655 | AGGCAGCA GGCTAGCTACAACGA AGAAGAAC | 5452 |
| 2649 | UCUUCUGU G CUGCCUGG | 656 | CCAGGCAG GGCTAGCTACAACGA ACAGAAGA | 5453 |
| 2652 | UCUGUGCU G CCUGGUAC | 657 | GTACCAGG GGCTAGCTACAACGA AGCACAGA | 5454 |
| 2657 | GCUGCCUG G UACAUCAA | 658 | TTGATGTA GGCTAGCTACAACGA CAGGCAGC | 5455 |
| 2659 | UGCCUGGU A CAUCAAAG | 659 | CTTTGATG GGCTAGCTACAACGA ACCAGGCA | 5456 |
| 2661 | CCUGGUAC A UCAAAGGC | 660 | GCCTTTGA GGCTAGCTACAACGA GTACCAGG | 5457 |
| 2668 | CAUCAAAG G CAAGCUGG | 661 | CCAGCTTG GGCTAGCTACAACGA CTTTGATG | 5458 |
| 2672 | AAAGGCAA G CUGGUCCC | 662 | GGGACCAG GGCTAGCTACAACGA TTGCCTTT | 5459 |
| 2676 | GCAAGCUG G UCCCUGGC | 663 | CCCAGGGA GGCTAGCTACAACGA CAGCTTGC | 5460 |
| 2685 | UCCCUGGG G CGGCAUAU | 664 | ATATUCCG GGCTAGCTACAACGA CCCAGGGA | 5461 |
| 2688 | CUUUGGCG G CAUAUGCU | 665 | AGCATATG GGCTAGCTACAACGA CGCCCCAU | 5462 |
| 2690 | GGGGCGGC A UAUUCUCU | 666 | AGAUCATA GGCTAGCTACAACGA GCCGCCCC | 5463 |
| 2692 | UUCUGCAU A UUCUCUCU | 667 | AGAGAUCA GGCTAGCTACAACGA ATGCCGCC | 5464 |
| 2694 | CUUCAUAU G CUCUCUAC | 668 | GTAGAGAG GGCTAUCTACAACGA ATATUCCG | 5465 |
| 2701 | UGCUCUCU A CUUCUUAU | 669 | ATACGCCG GGCTAGCTACAACGA AGAGAUCA | 5466 |
| 2704 | UCUCUACG G CUUAUGGC | 670 | GCCATACG GGCTAGCTACAACGA CUTAGAGA | 5467 |
| 2706 | UCUACGGC G UAUGGCCG | 671 | CGGCCATA GGCTAGCTACAACGA UCCUTAGA | 5468 |
| 2708 | UACGUCGU A UUUCCGCU | 672 | AGTAGCGG GGCTAGCTACAACGA ACUCCUTA | 5469 |
| 2711 | GUCUUAUU G CCGCUACU | 673 | AGTAGCGG GGCTAGCTACAACGA CATACGCC | 5470 |
| 2714 | UUAUUUCC G CUACUCCU | 674 | AGGAGTAG GGCTAGCTACAACGA GUCCATAC | 5471 |
| 2717 | UUUCCGCU A CUCCUGCU | 675 | AGCAGGAG GGCTAGCTACAACGA AGCGGCCA | 5472 |
| 2723 | CUACUCCU G CUCCUGCU | 676 | AGCAGGAG GGCTAGCTACAACGA AGGAUTAG | 5473 |
| 2729 | CUGCUCCU G CUGGCGUU | 677 | AACGCCAG GGCTAGCTACAACGA AUGAUCAG | 5474 |
| 2733 | UCCUGCUG G CGUUACCA | 678 | TGGTAACG GGCTAGCTACAACGA CAUCAGGA | 5475 |
| 2735 | CUUCUGUC G UUACCACC | 679 | GGTGGTAA GGCTAGCTACAACGA UCCAUCAG | 5476 |
| 2738 | CUGGCGUU A CCACCACG | 680 | CGTGGTGG GGCTAGCTACAACGA AACGCCAG | 5477 |
| 2741 | GCGUUACC A CCACGGGC | 681 | GCCCGTGG GGCTAGCTACAACGA GGTAACGC | 5478 |
| 2744 | UUACCACC A CGGGCGUA | 682 | TACGCCCG GGCTAGCTACAACGA GGTGGTAA | 5479 |
| 2748 | CACCACGG G CGUACGCC | 683 | GGCGTACG GGCTAGCTACAACGA CCGTGGTG | 5480 |
| 2750 | CCACGGGC G UACGCCAU | 684 | ATGGCGTA GGCTAGCTACAACGA GCCCGTGG | 5481 |
| 2752 | ACGGGCGU A CGCCAUGG | 685 | CCATCCCC GGCTAGCTACAACGA ACCCCCGT | 5482 |
| 2754 | GGGCGUAC G CCAUGGAC | 686 | GTCCATGG GGCTAGCTACAACGA GTACGCCC | 5483 |
| 2757 | CGUACGCC A UGGACCGG | 687 | CCGGTCGA GGCTAGCTACAACGA GGCGTACG | 5484 |
| 2761 | CGCCAUCC A CCGGGAGA | 688 | TCTCCCGG GGCTAGCTACAACGA CCATGGCG | 5485 |
| 2769 | ACCGGGAG A UGGCCGCA | 689 | TGCGGCGA GGCTAGCTACAACGA CTCCCGGT | 5486 |
| 2772 | GGGAGAUG G CCGCAUCG | 690 | CGATGCGG GGCTAGCTACAACGA CATCTCCC | 5487 |
| 2775 | AGAUGGCC G CAUCGUGC | 691 | GCACGATG GGCTAGCTACAACGA GGCCATCT | 5488 |
| 2777 | AUGGCCGC A UCGUGCGG | 692 | CCGCACGA GGCTAGCTACAACGA GCGGCCAT | 5489 |
| 2780 | GCCGCAUC G UGCGCACC | 693 | CCTCCCGA GGCTAGCTACAACGA GATGCGGC | 5490 |
| 2782 | CGCAUCCU G CGGAGCCC | 694 | CCCCTCCG GGCTAGCTACAACGA ACGATGCG | 5491 |
| 2788 | GUGCGGAG G CGUGCUUU | 695 | AAACCACG GGCTAGCTACAACGA CTCCGCAC | 5492 |
| 2790 | GCGGAGGC G UGGUUUUU | 696 | AAAAACGA GGCTAGCTACAACGA GCCTCCGC | 5493 |
| 2793 | GAGGCGUG G UUUUUGUA | 697 | TACAAAAA GGCTAGCTACAACGA CACGCCTC | 5494 |
| 2799 | UGCUUUUU G UACGUCUA | 698 | TAGACCTA GGCTAGCTACAACGA AAAAACCA | 5495 |
| 2803 | UUUUGUAG G UCUACCAC | 699 | CTCCTACA GGCTAGCTACAACGA CTACAAAA | 5496 |
| 2808 | UAGGUCUA G CACUCUUG | 700 | CAAGAGTC GGCTAGCTACAACGA TAGACCTA | 5497 |
| 2810 | GCUCUAGC A CUCUUCAC | 701 | GTCAAGAG GGCTAGCTACAACGA GCTAGACC | 5498 |
| 2817 | CACUCUUG A CCUUCUCA | 702 | TCACAACC GGCTAGCTACAACGA CAAGACTC | 5499 |
| 2822 | UUGACCUU G UCACCAUA | 703 | TATCCTCA GGCTAGCTACAACGA AAGCTCAA | 5500 |
| 2825 | ACCUUGUC A CCAUACUA | 704 | TAGTATCG GGCTAGCTACAACGA GACAACCT | 5501 |
| 2828 | UUGUCACC A UACUACAA | 705 | TTGTACTA GGCTAGCTACAACGA CGTCACAA | 5502 |
| 2830 | CUCACCAU A CUACAAAC | 706 | CTTTGTAG GGCTAGCTACAACGA ATGGTGAC | 5503 |
| 2833 | ACCAUACU A CAAAGUCU | 707 | ACACTTTG GGCTAGCTACAACGA AGTATCGT | 5504 |
| 2838 | ACUACAAA G UCUUCCUC | 708 | GAGCAACA GGCTAGCTACAACGA TTTGTAGT | 5505 |
| 2840 | UACAAAGU G UUCCUCGC | 709 | CCCACGAA GGCTAGCTACAACGA ACTTTCTA | 5506 |
| 2847 | UCUUCCUC G CUAGGCUC | 710 | CAGCCTAC GGCTAGCTACAACGA GACCAACA | 5507 |
| 2852 | CUCGCUAG G CUCAUAUG | 711 | CATATCAG GGCTAGCTACAACGA CTACCGAC | 5508 |
| 2856 | CUACCCUC A UAUCGUCC | 712 | CCACCATA GGCTAGCTACAACGA CAGCCTAC | 5509 |
| 2858 | AGCCUCAU A UCCUCCUU | 713 | AACCACGA GGCTAGCTACAACGA ATGAGCCT | 5510 |
| 2861 | CUCAUAUG G UCCUUCCA | 714 | TGCAACGA GGCTAGCTACAACGA CATATGAG | 5511 |
| 2864 | AUAUGCUC G UUCCAAUA | 715 | TATTCCAA GGCTAGCTACAACGA CACCATAT | 5512 |
| 2867 | UCCUCCUU G CAAUACCU | 716 | ACCTATTC GGCTAGCTACAACGA AACCACCA | 5513 |
| 2870 | UCCUUCCA A UACCUUAU | 717 | ATAACCTA GGCTAGCTACAACGA TCCAACCA | 5514 |
| 2872 | CUUCCAAU A CCUUAUCA | 718 | TCATAACC GGCTAGCTACAACGA ATTCCAAC | 5515 |
| 2877 | AAUACCUU A UCACCAGA | 719 | TCTCCTCA GGCTAGCTACAACGA AAGGTATT | 5516 |
| 2880 | ACCUUAUC A CCACACCC | 720 | CCCTCTCC GGCTAGCTACAACGA CATAACCT | 5517 |
| 2886 | UCACCACA G CCCACCCC | 721 | CCCCTCCC GGCTAGCTACAACGA TCTCCTCA | 5518 |
| 2892 | CACCCCAC G CCCACUUC | 722 | CAACTCCC GGCTAGCTACAACGA CTCCCCTC | 5519 |
| 2894 | CCCCACCC G CACUUCGA | 723 | TCCAACTC GGCTAGCTACAACGA CCCTCCCC | 5520 |
| 2897 | CACCCCGA G UUCCAAGU | 724 | ACTTCCAA GGCTAGCTACAACGA TCCCCCTC | 5521 |
| 2900 | CCCCACUU G CAACUCUC | 725 | CACACTTC GGCTAGCTACAACGA AACTGCGC | 5522 |
| 2904 | ACUUCCAA G UCUCCAUC | 726 | CATCCACA GGCTAGCTACAACGA TTCCAACT | 5523 |
| 2906 | UUCCAACU G UCCAUCCC | 727 | CCCATCGA GGCTAGCTACAACGA ACTTCCAA | 5524 |
| 2910 | AACUCUCC A UCCCCCCC | 728 | CCCCCCGA GGCTAGCTACAACGA CCACACTT | 5525 |
| 2923 | CCCCCUCA A CCUUCCCC | 729 | CCCCAACC GGCTAGCTACAACGA TCACCCCC | 5526 |
| 2925 | CCCUCAAC G UUCCGCCC | 730 | CCCCCGAA GGCTAGCTACAACGA CTTGACCG | 5527 |
| 2936 | CGGGGGGG G CCCCCUCC | 731 | CCACCCCC GGCTAGCTACAACGA CCCCCCCG | 5528 |
| 2938 | GGGGGGGC G CCCUGCGA | 732 | TGGCACCC GGCTAGCTACAACGA GCCCCCCC | 5529 |
| 2941 | GGGGCGCG G UCCCAUCA | 733 | TCATCCGA GGCTAGCTACAACGA CGCGCCCC | 5530 |
| 2943 | GGCGCGGU G CCAUCAUU | 734 | AATGATCC GGCTAGCTACAACGA ACCGCCCC | 5531 |
| 2946 | GCGGUGCC A UCAUUCUC | 735 | CACAATCA GGCTAGCTACAACGA GGCACCCG | 5532 |
| 2949 | GUGCCAUC A UUCUCCUC | 736 | CACCACAA GGCTAGCTACAACGA GATGGCAC | 5533 |
| 2958 | UUCUCCUC A CGUGUGUG | 737 | CACACACC GGCTAGCTACAACGA CAGGAGAA | 5534 |
| 2960 | CUCCUCAC G UGUGUGGU | 738 | ACCACACA GGCTAGCTACAACGA GTGAGGAG | 5535 |
| 2962 | CCUCACGU G UGUGGUCC | 739 | GGACCACA GGCTAGCTACAACGA ACGTGAGG | 5536 |
| 2964 | UCACGUGU G UGGUCCAC | 740 | GTGGACGA GGCTAGCTACAACGA ACACGTGA | 5537 |
| 2967 | CGUGUGUG G UCCACCGA | 741 | TGGGTGGA GGCTAGCTACAACGA CACACACG | 5538 |
| 2971 | UGUGGUCC A CCCAGAGC | 742 | GCTCTGGG GGCTAGCTACAACGA GGACCACA | 5539 |
| 2978 | CACCCAGA G CUAAUCUU | 743 | AAGATTAG GGCTAGCTACAACGA TCTGGGTG | 5540 |
| 2982 | CAGAGCUA A UCUUUGAC | 744 | GTCAAAGA GGCTAGCTACAACGA TAGCTCTG | 5541 |
| 2989 | AAUCUUUG A CAUCACGA | 745 | TGGTGATG GGCTAGCTACAACGA CAAAGATT | 5542 |
| 2991 | UCUUUGAC A UCACCAAA | 746 | TTTGGTGA GGCTAGCTACAACGA GTCAAAGA | 5543 |
| 2994 | UUGACAUC A CCAAAAUU | 747 | AATTTTGG GGCTAGCTACAACGA GATGTCAA | 5544 |
| 3000 | UCACCAAA A UUAUGCUC | 748 | GAGCATAA GGCTAGCTACAACGA TTTGGTGA | 5545 |
| 3003 | CCAAAAUU A UGCUCGCC | 749 | GGCGAGCA GGCTAGCTACAACGA AATTTTGG | 5546 |
| 3005 | AAAAUUAU G CUCGCCAU | 750 | ATGGCGAG GGCTAGCTACAACGA ATAATTTT | 5547 |
| 3009 | UUAUGCUC G CCAUACUC | 751 | GAGTATGG GGCTAGCTACAACGA GAGCATAA | 5548 |
| 3012 | UGCUCGCC A UACUCGGC | 752 | GCCGAGTA GGCTAGCTACAACGA GGCGAGCA | 5549 |
| 3014 | CUCGCCAU A CUCGGCCC | 753 | GGGCCGAG GGCTAGCTACAACGA ATGGCGAG | 5550 |
| 3019 | CAUACUCC G CCCGCUCA | 754 | TGAGCGGG GGCTAGCTACAACGA CGAGTATG | 5551 |
| 3023 | CUCGGCCC G CUCAUGGU | 755 | ACCATGAG GGCTAGCTACAACGA GGGCCGAG | 5552 |
| 3027 | CCCCGCUC A UGGUCCUC | 756 | GAGCACGA GGCTAGCTACAACGA GAGCGGGC | 5553 |
| 3030 | CGCUCAUG G UGCUCCAG | 757 | CTGGAGCA GGCTAGCTACAACGA CATGAGCG | 5554 |
| 3032 | CUCAUGGU G CUCCAGCC | 758 | GCCTGGAG GGCTAGCTACAACGA ACCATGAG | 5555 |
| 3039 | UGCUCCAG G CUGGUAUA | 759 | TATACCAG GGCTAGCTACAACGA CTGGAGCA | 5556 |
| 3043 | CCAGGCUG G UAUAGCAA | 760 | TTGCTATA GGCTAGCTACAACGA CAGCCTGG | 5557 |
| 3045 | AGGCUGGU A UAGCAAAA | 761 | TTTTGCTA GGCTAGCTACAACGA ACCAGCCT | 5558 |
| 3048 | CUGGUAUA G CAAAAGUG | 762 | CACTTTTG GGCTAGCTACAACGA TATACCAG | 5559 |
| 3054 | UAGCAAAA G UGCCGGAC | 763 | GTCCGGCA GGCTAGCTACAACGA TTTTGCTA | 5560 |
| 3056 | GCAAAACU G CCGCACUU | 764 | AAGTCCGG GGCTAGCTACAACGA ACTTTTGC | 5561 |
| 3061 | ACUGCCGG A CUUUGUGC | 765 | GCACAAAG GGCTAGCTACAACGA CCGGCACT | 5562 |
| 3066 | CCGACUUU G UGCGGGCU | 766 | AGCCCGCA GGCTAGCTACAACGA AAAGTCCG | 5563 |
| 3068 | GACUUUGU G CGGGCUCA | 767 | TGAGCCCG GGCTAGCTACAACGA ACAAAGTC | 5564 |
| 3072 | UUGUGCGG G CUCAAGGG | 768 | CCCTTGAG GGCTAGCTACAACGA CCGCACAA | 5565 |
| 3081 | CUCAAGGG G UCAUCCGU | 769 | ACGGATCA GGCTAGCTACAACGA CCCTTCAC | 5566 |
| 3084 | AACGGCUC A UCCGUGAA | 770 | TTCACCGA GGCTAGCTACAACGA GACCCCTT | 5567 |
| 3088 | GGUCAUCC G UGAAUGCA | 771 | TGCATTCA GGCTAGCTACAACGA GGATGACC | 5568 |
| 3092 | AUCCGUGA A UGCAUUUU | 772 | AAAATCGA GGCTAGCTACAACGA TCACGGAT | 5569 |
| 3094 | CCGUCAAU G CAUUUUGC | 773 | CCAAAATC GGCTAGCTACAACGA ATTCACGG | 5570 |
| 3096 | CUCAAUGC A UUUUGCUG | 774C | ACCAAAA GGCTAGCTACAACGA GCATTCAC | 5571 |
| 3102 | GCAUUUUG G UGCGGAAA | 775 | TTTCCGCA GGCTAGCTACAACGA CAAAATGC | 5572 |
| 3204 | AUUUUGGU G CGGAAAGU | 776 | ACTTTCCG GGCTAGCTACAACGA ACCAAAAT | 5573 |
| 3211 | UCCCCAAA G UCGCUGGC | 777 | CCCACCGA GGCTAGCTACAACGA TTTCCGCA | 5574 |
| 3215 | GAAAGUCG G UGGGGGGC | 778 | GCCCCCGA GGCTAGCTACAACGA CGACTTTC | 5575 |
| 3122 | GGUGGGGG G CAAUAUGU | 779 | ACATATTG GGCTAGCTACAACGA CCCCCACC | 5576 |
| 3125 | GGGGGGCA A UAUGUCGA | 780 | TGGACATA GGCTAGCTACAACGA TGCCCCCC | 5577 |
| 3127 | CCGCCAAU A UGUCCAAA | 781 | TTTGGACA GGCTAGCTACAACGA ATTGCCCC | 5578 |
| 3129 | GGCAAUAU G UCCAAAUG | 782 | CATTTGGA GGCTAGCTACAACGA ATATTGCC | 5579 |
| 3135 | AUCUCCAA A UGGCCUUC | 783 | GAACCCGA GGCTAGCTACAACGA TTGGACAT | 5580 |
| 3138 | UCCAAAUG G CCUUCAUG | 784 | CATCAAGG GGCTAGCTACAACGA CATTTGGA | 5581 |
| 3144 | UGGCCUUC A UCAAGUUG | 785 | CAACTTCA GGCTAGCTACAACGA GAAGGCCA | 5582 |
| 3149 | UUCAUCAA G UUGCCCGA | 786 | TCCCCCAA GGCTAGCTACAACGA TTCATCAA | 5583 |
| 3153 | UCAACUUC G CCCAAUUC | 787 | CAATTCCC GGCTAGCTACAACGA CAACTTCA | 5584 |
| 3158 | UUCCCCCA A UUCAAACC | 788 | CCTTTCAA GGCTAGCTACAACCA TCCCCCAA | 5585 |
| 3166 | AUUCAAAC G UACCUCCC | 789 | CCCACCTA GGCTAGCTACAACCA CTTTCAAT | 5586 |
| 3168 | UCAAAGCU A CCUCCCUC | 790 | CACGCACG GGCTAGCTACAACGA ACCTTTCA | 5587 |
| 3170 | AAACCUAC G UCCCUCUA | 791 | TACACCCA GGCTAGCTACAACCA CTACCTTT | 5588 |
| 3174 | CUACCUCC G UCUAUCAC | 792 | GTCATACA GGCTAGCTACAACCA CCACGTAC | 5589 |
| 3178 | GUCCCUCU A UCACCACC | 793 | CCTCGTCA GGCTAGCTACAACGA ACACCCAC | 5590 |
| 3182 | CGUCUAUC A CCACCUCA | 794 | TGAGGTGG GGCTAGCTACAACGA CATAGACG | 5591 |
| 3184 | CUAUGACC A CCUCACUC | 795 | GAGTGAGG GGCTAGCTACAACGA GGTCATAG | 5592 |
| 3189 | ACCACCUG A CUCCACUG | 796 | CAGTGGAG GGCTAGCTACAACGA GAGGTGGT | 5593 |
| 3194 | CUCACUCC A CUGCAGGA | 797 | TCCTGCAG GGCTAGCTACAACGA GGAGTGAG | 5594 |
| 3197 | ACUCCACU G CAGGACUG | 798 | CAGTCCTG GGCTAGCTACAACGA AGTGGAGT | 5595 |
| 3202 | ACUCCAGG A CUGGGCCC | 799 | GGGCCCAG GGCTAGCTACAACGA CCTGCAGT | 5596 |
| 3207 | AGGACUGG G CCCACACA | 800 | TGTGTGGG GGCTAGCTACAACGA CCAGTCCT | 5597 |
| 3211 | CUGGGCCC A CACAGGUC | 801 | GACCTGTG GGCTAGCTACAACGA GGGCCCAG | 5598 |
| 3213 | GGGCCCAC A CAGGUCUA | 802 | TAGACCTG GGCTAGCTACAACGA GTGGGCCC | 5599 |
| 3217 | CCACACAG G UCUACCAG | 803 | CTCGTAGA GGCTAGCTACAACGA CTGTGTGG | 5600 |
| 3221 | ACAGGUCU A CGAGACCU | 804 | AGGTCTCG GGCTAGCTACAACGA AGACCTGT | 5601 |
| 3226 | UCUACCAG A CCUGGCGG | 805 | CCGCCAGG GGCTAGCTACAACGA CTCGTAGA | 5602 |
| 3231 | GAGACCUG G CGGUACCC | 806 | CGCTACCG GGCTAGCTACAACGA CAGGTCTC | 5603 |
| 3234 | ACCUGGCG G UAGCGGUC | 807 | CACCGCTA GGCTAGCTACAACGA CGCCACGT | 5604 |
| 3237 | UGGCGGUA G CGGUCCAG | 808 | CTCGACCG GGCTAGCTACAACGA TACCGCCA | 5605 |
| 3240 | CGGUAGCG G UCGACCCC | 809 | GGGCTCGA GGCTAGCTACAACGA CCCTACCG | 5606 |
| 3245 | GCGGUCGA G CCCCUCGU | 810 | ACGACGCG GGCTAGCTACAACGA TCGACCGC | 5607 |
| 3249 | UCGACCCC G UCGUCUUC | 811 | GAACACGA GGCTAGCTACAACGA CGGCTCGA | 5608 |
| 3252 | ACCCCGUC G UCUUCUCC | 812 | GGACAACA GGCTAGCTACAACGA GACGCGCT | 5609 |
| 3262 | CUUCUCCG A CAUGGAAA | 813 | TTTCCATG GGCTAGCTACAACGA CGGAGAAG | 5610 |
| 3264 | UCUCCGAC A UGGAAAUC | 814 | GATTTCGA GGCTAGCTACAACGA GTCCGAGA | 5611 |
| 3270 | ACAUCGAA A UCAACAUC | 815 | GATCTTGA GGCTAGCTACAACGA TTCCATGT | 5612 |
| 3276 | AAAUCAAG A UCAUCACC | 816 | CGTGATCA GGCTAGCTACAACGA CTTCATTT | 5613 |
| 3279 | UCAAGAUC A UCACCUGG | 817 | CCAGGTCA GGCTAGCTACAACGA GATCTTGA | 5614 |
| 3282 | AGAUCAUC A CCUGGGGC | 818 | CCCCCAGG GGCTAGCTACAACGA GATCATCT | 5615 |
| 3295 | GGGGGGAC A CACCGCCG | 819 | CCGCGGTG GGCTAGCTACAACGA CTCCCCCC | 5616 |
| 3297 | GGCCAGAC A CCGCGCCG | 820 | CGCCCCCC CCCTAGCTACAACGA GTCTCCCC | 5617 |
| 3300 | GAGACACC G CGCCCUGU | 821 | ACACGCCG GGCTAGCTACAACGA GGTGTCTC | 5618 |
| 3303 | ACACCCCG G CGUGUCGG | 822 | CCCACACG GGCTAGCTACAACGA CGCGGTGT | 5619 |
| 3305 | ACCGCGGC G UGUGGGGA | 823 | TCCCCACA GGCTAGCTACAACGA GCCGCGGT | 5620 |
| 3307 | CCCGCCGU G UCGCCACA | 824 | TGTCCCGA GGCTAGCTACAACGA ACGCCGCG | 5621 |
| 3313 | GUGUGCGG A CAUCAUUA | 825 | TAATCATG GGCTAGCTACAACGA CCCCACAC | 5622 |
| 3315 | GUGGCGAC A UCAUUAUG | 826 | CATAATGA GGCTAGCTACAACGA GTCCCCAC | 5623 |
| 3318 | GGGACAUC A UUAUGGCU | 827 | ACCCATAA GGCTAGCTACAACGA GATCTCCC | 5624 |
| 3321 | ACAUCAUU A UGGGUCUA | 828 | TACACCGA GGCTAGCTACAACGA AATGATGT | 5625 |
| 3325 | CAUUAUCG G UCUACCUG | 829 | CAGGTACA GGCTAGCTACAACGA CCATAATG | 5626 |
| 3329 | AUGGGUCU A CCUGUCUC | 830 | GAGACAGG GGCTAGCTACAACGA AGACCCAT | 5627 |
| 3333 | CUCUACCU G UCUCCGCC | 831 | CGCGCAGA GGCTAGCTACAACGA AGGTAGAC | 5628 |
| 3339 | CUGUCUCC G CCCCAACC | 832 | CCTTCCCC GGCTAGCTACAACGA CCACACAC | 5629 |
| 3357 | GCACGCAG A UACUCCUA | 833 | TAGGACTA GGCTAGCTACAACGA CTCCCTCC | 5630 |
| 3359 | AGGCAGAU A CUCCUACC | 834 | CCTACGAG GGCTAGCTACAACGA ATCTCCCT | 5631 |
| 3368 | CUCCUACG A CCACCCGA | 835 | TCCCCTCC GGCTAGCTACAACGA CCTAGCAC | 5632 |
| 3372 | UACCACGA G CCCACACU | 836 | ACTCTCCC GGCTAGCTACAACGA TCCTCCTA | 5633 |
| 3376 | ACCACCCG A CAGUCUUG | 837 | CAACACTG GGCTAGCTACAACGA CGGCTCCT | 5634 |
| 3379 | ACCCCACA G UCUUGAGG | 838 | CCTCAACA CCCTAGCTACAACGA TCTCCCCT | 5635 |
| 3389 | CUUCACCC G CACGGCUG | 839 | CACCCCTC CCCTAGCTACAACGA CCCTCAAC | 5636 |
| 3395 | CCCCACCC G UCCCCACU | 840 | ACTCCCGA CCCTAGCTACAACGA CCCTCCCC | 5637 |
| 3398 | CAGGGGUG G CGACUCCU | 841 | AGGAGTCG GGCTAGCTACAACGA CACCCCTG | 5638 |
| 3401 | GCGUGGCG A CUCCUCGC | 842 | GCGACGAG GGCTAGCTACAACGA CCCCACCC | 5639 |
| 3408 | GACUCCUC G CGCCCAUU | 843 | AATGGGCG GGCTAGCTACAACGA CACGAGTC | 5640 |
| 3410 | CUCCUCCC G CCCAUUAC | 844 | CTAATGCC CCCTAGCTACAACGA CCCACGAC | 5641 |
| 3414 | UCGCGCCC A UUACGGCC | 845 | GGCCCTAA GGCTAGCTACAACGA CGGCGCGA | 5642 |
| 3417 | CGCCCAUU A CGGCCUAC | 846 | GTACCCCC GGCTAGCTACAACGA AATCCCCC | 5643 |
| 3420 | CCAUUACC G CCUACUCC | 847 | CCACTAGC GGCTAGCTACAACGA CCTAATGC | 5644 |
| 3424 | UACGCCCU A CUCCCAAC | 848 | GTTCCCAG GGCTAGCTACAACGA ACCCCCTA | 5645 |
| 3431 | UACUCCCA A CACACCCC | 849 | CCCCTCTC CCCTAGCTACAACGA TCGCAGTA | 5646 |
| 3435 | CCCAACAC A CCCCCGCC | 850 | GCCCCGCG CCCTAGCTACAACGA CTCTTCCG | 5647 |
| 3437 | CAACACAC G CGGGCCCU | 851 | AGCCCCCG GGCTAGCTACAACGA CTCTCTTG | 5648 |
| 3442 | GACGCGGG G CCUGUUUG | 852 | CAAACAGG GGCTAGCTACAACGA CCCGCGTC | 5649 |
| 3446 | CGGGGCCU G UUUGGCUG | 853 | CAGCCAAA GGCTAGCTACAACGA AGGCCCCG | 5650 |
| 3451 | CCUGUUUG G CUGCAUUA | 854 | TAATGCAG GGCTAGCTACAACGA CAAACAGG | 5651 |
| 3454 | GUUUGGCU G CAUUAUCA | 855 | TGATAATG GGCTAGCTACAACGA AGCCAAAC | 5652 |
| 3456 | UUGGCUGC A UUAUCACC | 856 | GGTGATAA GGCTAGCTACAACGA GCAGCCAA | 5653 |
| 3459 | GCUGCAUU A UCACCAGC | 857 | GCTGGTGA GGCTAGCTACAACGA AATGCAGC | 5654 |
| 3462 | GCAUUAUC A CCAGCCUC | 858 | GAGGCTGG GGCTAGCTACAACGA GATAATGC | 5655 |
| 3466 | UAUCACGA G CCUCACGG | 859 | CCGTGAGG GGCTAGCTACAACGA TGGTGATA | 5656 |
| 3471 | CCAGCCUC A CGGGCCGG | 860 | CCGGCCCG GGCTAGCTACAACGA GAGGCTGG | 5657 |
| 3475 | CCUCACGG G CCGGGACA | 861 | TGTCCCGG GGCTAGCTACAACGA CCGTGAGG | 5658 |
| 3481 | GGGCCGGG A CAAGAACC | 862 | GGTTCTTG GGCTAGCTACAACGA CCCGGCCC | 5659 |
| 3487 | GGACAAGA A CCAAGUCG | 863 | CGACTTGG GGCTAGCTACAACGA TCTTGTCC | 5660 |
| 3492 | AGAACCAA G UCGAGGGG | 864 | CCCCTCGA GGCTAGCTACAACGA TTGGTTCT | 5661 |
| 3504 | AGGGGGAA G UUCAAGUG | 865 | CACTTGAA GGCTAGCTACAACGA TTCCCCCT | 5662 |
| 3510 | AAGUUCAA G UGGUUUCC | 866 | GGAAACGA GGCTAGCTACAACGA TTGAACTT | 5663 |
| 3513 | UUCAAGUG G UUUCCACC | 867 | GGTGGAAA GGCTAGCTACAACGA CACTTGAA | 5664 |
| 3519 | UGGUUUCC A CCGCGACG | 868 | CGTCGCGG GGCTAGCTACAACGA GGAAACCA | 5665 |
| 3522 | UUUCCACC G CGACGCAG | 869 | CTGCGTCG GGCTAGCTACAACGA GGTGGAAA | 5666 |
| 3525 | CCACCGCG A CGCAGUCU | 870 | AGACTGCG GGCTAGCTACAACGA CGCGGTGG | 5667 |
| 3527 | ACCGCGAC G CAGUCUUU | 871 | AAAGACTG GGCTAGCTACAACGA GTCGCGGT | 5668 |
| 3530 | GCGACGCA G UCUUUCCU | 872 | AGGAAAGA GGCTAGCTACAACGA TGCGTCGC | 5669 |
| 3540 | CUUUCCUA G CGACCUCC | 873 | GCAGGTCG GGCTAGCTACAACGA TAGGAAAG | 5670 |
| 3543 | UCCUAGCG A CCUGCGUC | 874 | GACCCAGG GGCTAGCTACAACGA GGCTAGGA | 5671 |
| 3547 | AGCCACCU G CGUCAACG | 875 | CGTTGACG GGCTAGCTACAACGA AGGTCGCT | 5672 |
| 3549 | CGACCUGC G UCAACGGC | 876 | GCCGTTCA GGCTAGCTACAACGA GCAGCTCC | 5673 |
| 3553 | CUCCGUCA A CCGCGUGU | 877 | ACACCCCG GGCTAGCTACAACGA TCACGCAG | 5674 |
| 3556 | CGUCAACG G CGUGUGCU | 878 | AGCACACG GGCTAGCTACAACGA CGTTGACG | 5675 |
| 3558 | UCAACGCC G UGUCCUGC | 879 | CCAGCACA CCCTAGCTACAACGA CCCGTTCA | 5676 |
| 3560 | AACCGCGU G UGCUCCAC | 880 | GTCCACGA GGCTAGCTACAACGA ACGCCCTT | 5677 |
| 3562 | CCGCGUCU G CUGGACUG | 881 | CAGTCCAG GGCTAGCTACAACGA ACACGCCG | 5678 |
| 3567 | UGUGCUGG A CUGUCUAC | 882 | CTAGACAG GGCTAGCTACAACGA CCACCACA | 5679 |
| 3570 | CCUCCACU G UCUACCAC | 883 | CTGCTACA GGCTAGCTACAACGA ACTCCAGC | 5680 |
| 3574 | GACUCUCU A CCACGCCC | 884 | CGCCGTGG GGCTAGCTACAACGA AGACAGTC | 5681 |
| 3577 | UCUCUACC A CGCCGCCC | 885 | CCGCGCCC GGCTAGCTACAACGA CGTAGACA | 5682 |
| 3580 | CUACCACG G CGCCGCCU | 886 | AGCCCGCG GGCTAGCTACAACGA CGTGGTAG | 5683 |
| 3582 | ACCACCCC G CCCCCUCA | 887 | TCAGCCCG GGCTAGCTACAACGA GCCCTCGT | 5684 |
| 3586 | CGCCGCCC G CUCAAAGA | 888 | TCTTTGAG GGCTAGCTACAACGA CGCCGCCG | 5685 |
| 3594 | CCUCAAAC A CCCUAGCC | 889 | CGCTACGC CCCTAGCTACAACGA CTTTGAGC | 5686 |
| 3600 | AGACCCUA G CCGGCCCA | 890 | TCCCCCGG GGCTAGCTACAACGA TAGGGTCT | 5687 |
| 3604 | CCUAGCCG G CCCAAACC | 891 | CCTTTGCG GGCTAGCTACAACGA CGCCTACG | 5688 |
| 3613 | CCCAAAGG G UCCAAUCA | 892 | TGATTCGA GGCTAGCTACAACGA CCTTTGGC | 5689 |
| 3618 | AGGGUCCA A UCACCCAA | 893 | TTGGGTGA GGCTAGCTACAACGA TGGACCCT | 5690 |
| 3621 | GUCCAAUC A CCCAAAUC | 894 | CATTTGCC GGCTAGCTACAACGA GATTGCAC | 5691 |
| 3627 | UCACCCAA A UGUACACC | 895 | GGTGTACA GGCTAGCTACAACGA TTGGGTGA | 5692 |
| 3629 | ACCCAAAU G UACACCAA | 896 | TTCGTGTA GGCTAGCTACAACGA ATTTGCGT | 5693 |
| 3631 | CCAAAUGU A CACCAAUG | 897 | CATTCGTG GGCTAGCTACAACGA ACATTTGG | 5694 |
| 3633 | AAAUGUAC A CCAAUCUA | 898 | TACATTGG GGCTAGCTACAACGA CTACATTT | 5695 |
| 3637 | CUACACCA A UCUAGACC | 899 | CCTCTACA CCCTAGCTACAACGA TCGTGTAC | 5696 |
| 3639 | ACACCAAU G UACACCAG | 900 | CTCCTCTA CCCTAGCTACAACGA ATTCCTCT | 5697 |
| 3643 | CAAUCUAC A CCAGGACC | 901 | CCTCCTCC CCCTAGCTACAACGA CTACATTC | 5698 |
| 3649 | ACACCACC A CCUCCUCC | 902 | CCACCACC GGCTAGCTACAACGA CCTCCTCT | 5699 |
| 3654 | ACCACCUC G UCCCAUGG | 903 | CCATCCGA CCCTAGCTACAACGA CACCTCCT | 5700 |
| 3659 | CUCGUCGG A UGGCCGGC | 904 | GCCGGCGA GGCTAGCTACAACGA CCCACGAG | 5701 |
| 3662 | GUCGGAUG G CCGGCGCC | 905 | GGCGCCGG GGCTAGCTACAACGA CATCCGAC | 5702 |
| 3666 | GAUGGCCG G CGCCCCCC | 906 | GGGGGGCG CCCTAGCTACAACGA CGGCCATC | 5703 |
| 3668 | UGGCCGGC G CCCCCCGG | 907 | CCGGGGGG GGCTAGCTACAACGA GCCGGCCA | 5704 |
| 3678 | CCCCCGGA G CGCGGUCC | 908 | GGACCGCG GGCTAGCTACAACGA TCCGGGGG | 5705 |
| 3680 | CCCGGAGC G CGGUCCUU | 909 | AAGGACCG GGCTAGCTACAACGA GCTCCGGG | 5706 |
| 3683 | GGAGCGCG G UCCUUGAC | 910 | GTCAAGGA GGCTAGCTACAACGA CGCGCTCC | 5707 |
| 3690 | GGUCCUUG A CACCAUGC | 911 | GCATGGTG GGCTAGCTACAACGA CAAGGACC | 5708 |
| 3692 | UCCUUGAC A CCAUGCAC | 912 | GTGCATGG GGCTAGCTACAACGA GTCAAGGA | 5709 |
| 3695 | UUGACACC A UGCACCUG | 913 | CAGGTGCA GGCTAGCTACAACGA GGTGTCAA | 5710 |
| 3697 | GACACCAU G CACCUGCG | 914 | CGCAGGTG GGCTAGCTACAACGA ATGGTGTC | 5711 |
| 3699 | CACCAUGC A CCUGCGGC | 915 | GCCGCAGG GGCTAGCTACAACGA GCATGGTG | 5712 |
| 3703 | AUGCACCU G CGGCGGCU | 916 | AGCCGCCG GGCTAGCTACAACGA AGGTGCAT | 5713 |
| 3706 | CACCUGCG G CGGCUCGG | 917 | CCGAGCCG GGCTAGCTACAACGA CGCAGGTG | 5714 |
| 3709 | CUGCGGCG G CUCGGACC | 918 | GGTCCGAG GGCTAGCTACAACGA CGCCGCAG | 5715 |
| 3715 | CGGCUCGG A CCUUUACU | 919 | AGTAAAGG GGCTAGCTACAACGA CCGAGCCG | 5716 |
| 3721 | GGACCUUU A CUUGGUCA | 920 | TGACCAAG GGCTAGCTACAACGA AAAGGTCC | 5717 |
| 3726 | UUUACUUG G UCACGAGA | 921 | TCTCGTGA GGCTAGCTACAACGA CAAGTAAA | 5718 |
| 3729 | ACUUGGUC A CGACACAC | 922 | GTGTCTCG GGCTAGCTACAACGA GACCAAGT | 5719 |
| 3734 | GUCACGAG A CACGCUGA | 923 | TCAGCCTG GGCTAGCTACAACGA CTCGTGAC | 5720 |
| 3736 | CACGAGAC A CGCUGAUG | 924 | CATCAGCG GGCTAGCTACAACGA GTCTCGTG | 5721 |
| 3738 | CGAGACAC G CUGAUGUC | 925 | GACATCAG GGCTAGCTACAACGA GTGTCTCG | 5722 |
| 3742 | ACACGCUG A UGUCAUUC | 926 | GAATGACA GGCTAGCTACAACGA CAGCGTGT | 5723 |
| 3744 | ACGCUGAU G UCAUUCCG | 927 | CGGAATGA GGCTAGCTACAACGA ATCAGCGT | 5724 |
| 3747 | CUGAUGUC A UUCCGGUG | 928 | CACCGGAA GGCTAGCTACAACGA GACATCAG | 5725 |
| 3753 | UCAUUCCG G UGCGCCGG | 929 | CCGGCGCA GGCTAGCTACAACGA CGGAATGA | 5726 |
| 3755 | AUUCCGGU G CGCCGGCG | 930 | CGCCGGCG GGCTAGCTACAACGA ACCGGAAT | 5727 |
| 3757 | UCCGGUGC G CCGGCGGG | 931 | CCCGCCGG GGCTAGCTACAACGA GCACCGGA | 5728 |
| 3761 | GUGCGCCG G CGGGGUGA | 932 | TCACCCCG GGCTAGCTACAACGA CGGCGCAC | 5729 |
| 3766 | CCGGCGGG G UGACAGCA | 933 | TGCTGTCA GGCTAGCTACAACGA CCCGCCGG | 5730 |
| 3769 | GCGGGGUG A CAGCAGGG | 934 | CCCTGCTG GGCTAGCTACAACGA CACCCCGC | 5731 |
| 3772 | GGGUGACA G CAGGGGGA | 935 | TCCCCCTG GGCTAGCTACAACGA TGTCACCC | 5732 |
| 3781 | CAGGGGGA G CUUACUAU | 936 | ATAGTAAG GGCTAGCTACAACGA TCCCCCTG | 5733 |
| 3785 | GGGAGCUU A CUAUCCCC | 937 | GGGGATAG GGCTAGCTACAACGA AAGCTCCC | 5734 |
| 3788 | AGCUUACU A UCCCCCAG | 938 | CTGGGGGA GGCTAGCTACAACGA AGTAAGCT | 5735 |
| 3797 | UCCCCCAG G CCCAUCUC | 939 | GAGATGGG GGCTAGCTACAACGA CTGGGGGA | 5736 |
| 3801 | CCAGGCCC A UCUCCUAC | 940 | GTAGGAGA GGCTAGCTACAACGA GGGCCTGG | 5737 |
| 3808 | CAUCUCCU A CUUGAAGG | 941 | CCTTCAAG GGCTAGCTACAACGA AGGAGATG | 5738 |
| 3817 | CUUGAAGG G CUCCUCGG | 942 | CCGAGGAG GGCTAGCTACAACGA CCTTCAAG | 5739 |
| 3826 | CUCCUCGG G CGGUCCAC | 943 | GTGGACCG GGCTAGCTACAACGA CCGAGGAG | 5740 |
| 3829 | CUCGGGCG G UCCACUGC | 944 | GCAGTGGA GGCTAGCTACAACGA CGCCCGAG | 5741 |
| 3833 | GGCGGUCC A CUGCUCUG | 945 | CAGAGCAG GGCTAGCTACAACGA GGACCGCC | 5742 |
| 3836 | GGUCCACU G CUCUGCCC | 946 | GGGCAGAG GGCTAGCTACAACGA AGTCGACC | 5743 |
| 3841 | ACUGCUCU G CCCUUCGG | 947 | CCGAAGGC GGCTAGCTACAACGA ACAGCAGT | 5744 |
| 3851 | CCUUCGGG G CACGUUCU | 948 | ACAACGTG GGCTAGCTACAACGA CCCGAACC | 5745 |
| 3853 | UUCGGGGC A CGUUGUGG | 949 | CCACAACG GGCTAGCTACAACGA GCCCCGAA | 5746 |
| 3855 | CGGGGCAC G UUGUGGGC | 950 | GCCCACAA GGCTAGCTACAACGA GTGCCCCG | 5747 |
| 3858 | GGCACGUU G UGGCCAUC | 951 | GATGCCGA GGCTAGCTACAACGA AACGTGCC | 5748 |
| 3862 | CGUUGUGG G CAUCUUCC | 952 | CGAAGATG GGCTAGCTACAACGA CCACAACG | 5749 |
| 3864 | UUGUGGGC A UCUUCCGG | 953 | CCGGAAGA GGCTAGCTACAACGA GCCCACAA | 5750 |
| 3873 | UCUUCCGG G CUCCUGUG | 954 | CACACCAC GGCTAGCTACAACGA CCGGAAGA | 5751 |
| 3876 | UCCGCCCU G CUGUCUCC | 955 | GCACACAG GGCTAGCTACAACGA AGCCCGGA | 5752 |
| 3879 | CGGCUGCU G UGUGCACC | 956 | GGTCCACA GGCTAGCTACAACGA ACCAGCCC | 5753 |
| 3881 | CCUCCUGU G UCCACCCG | 957 | CCGGTGCA GGCTAGCTACAACGA ACAGCACC | 5754 |
| 3883 | UCCUGUGU G CACCCGCG | 958 | CCCGGGTC GGCTAGCTACAACGA ACACAGCA | 5755 |
| 3885 | CUGUGUGC A CCCGCGGG | 959 | CCCCCCGG GGCTAGCTACAACGA CCACACAG | 5756 |
| 3894 | CCCGCGGG G UUGCGAAG | 960 | CTTCGCAA GGCTAGCTACAACGA CCCCCGGG | 5757 |
| 3897 | GGGCGCUU G CGAAGCCC | 961 | CCCCTTCC GGCTAGCTACAACGA AACCCCCC | 5758 |
| 3903 | UUGCGAAG G CGGUCGAC | 962 | GTCCACCG GGCTAGCTACAACGA CTTCGCAA | 5759 |
| 3906 | CGAAGGCG G UGGACUUU | 963 | AAAGTCGA GGCTAGCTACAACGA CGCCTTCC | 5760 |
| 3910 | CCCGCUGG A CUUUGUAC | 964 | GTACAAAG GGCTAGCTACAACGA CCACCGCC | 5761 |
| 3915 | UGGACUUU G UACCCGUU | 965 | AACGGGTA GGCTAGCTACAACGA AAAGTCCA | 5762 |
| 3917 | GACUUUGU A CCCGUUGA | 966 | TCAACGGG GGCTAGCTACAACGA ACAAAGTC | 5763 |
| 3921 | UUGAUCCC G UUGAGUCU | 967 | AGACTCAA GGCTAGCTACAACGA GGGTACAA | 5764 |
| 3926 | CCCGUUGA G UCUAUGGA | 968 | TCCATAGA GGCTAGCTACAACGA TCAACGGG | 5765 |
| 3930 | UUGAGUCU A UGGAAACU | 969 | AGTTTCGA GGCTAGCTACAACGA AGACTCAA | 5766 |
| 3936 | CUAUGGAA A CUACCAUG | 970 | CATGGTAG GGCTAGCTACAACGA TTCCATAG | 5767 |
| 3939 | UGGAAACU A CCAUGCGG | 971 | CCGCATGG GGCTAGCTACAACGA AGTTTCCA | 5768 |
| 3942 | AAACUACC A UGCGGUCC | 972 | GGACCGCA GGCTAGCTACAACGA GGTAGTTT | 5769 |
| 3944 | ACUACCAU G CGGUCCCC | 973 | GGGGACCG GGCTAGCTACAACGA ATGGTAGT | 5770 |
| 3947 | ACCAUGCG G UCCCCGGU | 974 | ACCGGGGA GGCTAGCTACAACGA CGCATGGT | 5771 |
| 3954 | GGUCCCCG G UCUUCACG | 975 | CGTGAAGA GGCTAGCTACAACGA CGGGGACC | 5772 |
| 3960 | CGGUCUUC A CGGACAAC | 976 | GTTGTCCG GGCTAGCTACAACGA GAAGACCG | 5773 |
| 3964 | CUUCACGG A CAACUCGU | 977 | ACGAGTTG GGCTAGCTACAACGA CCGTGAAG | 5774 |
| 3967 | CACGGACA A CUCGUCCC | 978 | GGGACGAG GGCTAGCTACAACGA TGTCCGTG | 5775 |
| 3971 | GACAACUC G UCCCCCCC | 979 | GGGGGGGA GGCTAGCTACAACGA GAGTTGTC | 5776 |
| 3981 | CCCCCCGA G CCGUACCG | 980 | CGGTACGG GGCTAGCTACAACGA TGGGGGGG | 5777 |
| 3984 | CCCCAGCC G UACCGCAG | 981 | CTGCGGTA GGCTAGCTACAACGA GGCTGGGG | 5778 |
| 3986 | CCAGCCGU A CCGCAGAC | 982 | GTCTGCGG GGCTAGCTACAACGA ACGGCTGG | 5779 |
| 3989 | GCCGUACC G CAGACAUU | 983 | AATGTCTG GGCTAGCTACAACGA GGTACGGC | 5780 |
| 3993 | UACCGCAG A CAUUCCAA | 984 | TTGGAATG GGCTAGCTACAACGA CTGCGGTA | 5781 |
| 3995 | CCGCAGAC A UUCCAAGU | 985 | ACTTGGAA GGCTAGCTACAACGA GTCTGCGG | 5782 |
| 4002 | CAUUCCAA G UGGCCCAC | 986 | GTGGGCGA GGCTAGCTACAACGA TTGGAATG | 5783 |
| 4005 | UCCAAGUG G CCCACCUA | 987 | TAGGTGGG GGCTAGCTACAACGA CACTTGGA | 5784 |
| 4009 | AGUGGCCC A CCUACACG | 988 | CGTGTAGG GGCTAGCTACAACGA GGGCCACT | 5785 |
| 4013 | GCCCACCU A CACGCUCC | 989 | GGAGCGTG GGCTAGCTACAACGA AGGTGGGC | 5786 |
| 4015 | CCACCUAC A CGCUCCGA | 990 | TGGGAGCG GGCTAGCTACAACGA GTAGGTGG | 5787 |
| 4017 | ACCUACAC G CUCCCACU | 991 | AGTGGGAG GGCTAGCTACAACGA GTGTAGGT | 5788 |
| 4023 | ACGCUCCC A CUGGCAGC | 992 | GCTGCCAG GGCTAGCTACAACGA GGGAGCGT | 5789 |
| 4027 | UCCCACUG G CAGCGGCA | 993 | TGCCGCTG GGCTAGCTACAACGA CAGTGGGA | 5790 |
| 4030 | CACUGGCA G CGGCAAGA | 994 | TCTTGCCG GGCTAGCTACAACGA TGCCAGTG | 5791 |
| 4033 | UGGCAGCG G CAAGAGCA | 995 | TGCTCTTG GGCTAGCTACAACGA CGCTGCCA | 5792 |
| 4039 | CGGCAAGA G CACUAAGG | 996 | CCTTAGTG GGCTAGCTACAACGA TCTTGCCG | 5793 |
| 4041 | GCAAGAGC A CUAAGGUA | 997 | TACCTTAG GGCTAGCTACAACGA GCTCTTGC | 5794 |
| 4047 | GCACUAAG G UACCGGCU | 998 | AGCCGGTA GGCTAGCTACAACGA CTTAGTGC | 5795 |
| 4049 | ACUAAGGU A CCGGCUGC | 999 | GCAGCCGG GGCTAGCTACAACGA ACCTTAGT | 5796 |
| 4053 | AGGUACCG G CUGCAUAU | 1000 | ATATGCAG GGCTAGCTACAACGA CGGTACCT | 5797 |
| 4056 | UACCGGCU G CAUAUGCA | 1001 | TGCATATG GGCTAGCTACAACGA AGCCGGTA | 5798 |
| 4058 | CCGGCUGC A UAUGCAGC | 1002 | GCTGCATA GGCTAGCTACAACGA GCAGCCGG | 5799 |
| 4060 | GGCUGCAU A UGCAGCCC | 1003 | GGGCTGCA GGCTAGCTACAACGA ATGCAGCC | 5800 |
| 4062 | CUGCAUAU G CAGCCCAA | 1004 | TTGGGCTG GGCTAGCTACAACGA ATATGCAG | 5801 |
| 4065 | CAUAUGCA G CCCAAGGG | 1005 | CCCTTGGG GGCTAGCTACAACGA TGCATATG | 5802 |
| 4073 | GCCCAAGG G UACAAAGU | 1006 | ACTTTGTA GGCTAGCTACAACGA CCTTGGGC | 5803 |
| 4075 | CCAAGGGU A CAAAGUGC | 1007 | GCACTTTG GGCTAGCTACAACGA ACCCTTGG | 5804 |
| 4080 | GGUACAAA G UGCUCGUC | 1008 | GACGAGCA GGCTAGCTACAACGA TTTGTACC | 5805 |
| 4082 | UACAAAGU G CUCGUCCU | 1009 | AGGACGAG GGCTAGCTACAACGA ACTTTGTA | 5806 |
| 4086 | AAGUGCUC G UCCUAAAU | 1010 | ATTTAGGA GGCTAGCTACAACGA GAGCACTT | 5807 |
| 4093 | CGUCCUAA A UCCGUCCG | 1011 | CGGACGGA GGCTAGCTACAACGA TTAGGACG | 5808 |
| 4097 | CUAAAUCC G UCCGUUAC | 1012 | GTAACGGA GGCTAGCTACAACGA GGATTTAG | 5809 |
| 4101 | AUCCGUCC G UUACCGCC | 1013 | GGCGGTAA GGCTAGCTACAACGA GGACGGAT | 5810 |
| 4104 | CGUCCGUU A CCGCCACC | 1014 | GGTGGCGG GGCTAGCTACAACGA AACGGACG | 5811 |
| 4107 | CCGUUACC G CCACCUUA | 1015 | TAAGGTGG GGCTAGCTACAACGA GGTAACGG | 5812 |
| 4110 | UUACCGCC A CCUUAGGG | 1016 | CCCTAAGG GGCTAGCTACAACGA GGCGGTAA | 5813 |
| 4118 | ACCUUAGG G UUUGGGGC | 1017 | GCCCCAAA GGCTAGCTACAACGA CCTAAGGT | 5814 |
| 4125 | GGUUUGGG G CGUAUAUG | 1018 | CATATACG GGCTAGCTACAACGA CCCAAACC | 5815 |
| 4127 | UUUGGGGC G UAUAUGUC | 1019 | GACATATA GGCTAGCTACAACGA GCCCCAAA | 5816 |
| 4129 | UGGGGCGU A UAUGUCUA | 1020 | TAGACATA GGCTAGCTACAACGA ACGCCCCA | 5817 |
| 4131 | GGGCGUAU A UGUCUAAG | 1021 | CTTAGACA GGCTAGCTACAACGA ATACGCCC | 5818 |
| 4133 | GCGUAUAU G UCUAAGGC | 1022 | GCCTTAGA GGCTAGCTACAACGA ATATACGC | 5819 |
| 4140 | UGUCUAAG G CACACGGU | 1023 | ACCGTGTG GGCTAGCTACAACGA CTTAGACA | 5820 |
| 4142 | UCUAAGGC A CACGGUGU | 1024 | ACACCGTG GGCTAGCTACAACGA GCCTTAGA | 5821 |
| 4144 | UAAGGCAC A CGGUGUCG | 1025 | CGACACCG GGCTAGCTACAACGA GTGCCTTA | 5822 |
| 4147 | GGCACACG G UGUCGAUC | 1026 | GATCGACA GGCTAGCTACAACGA CGTGTGCC | 5823 |
| 4149 | CACACGGU G UCGAUCCU | 1027 | AGGATCGA GGCTAGCTACAACGA ACCGTGTG | 5824 |
| 4153 | CGGUGUCG A UCCUAACA | 1028 | TGTTAGGA GGCTAGCTACAACGA CGACACCG | 5825 |
| 4159 | CGAUCCUA A CAUCAGAA | 1029 | TTCTGATG GGCTAGCTACAACGA TAGGATCG | 5826 |
| 4161 | AUCCUAAC A UCAGAACU | 1030 | AGTTCTGA GGCTAGCTACAACGA GTTAGGAT | 5827 |
| 4167 | ACAUCAGA A CUGGGGUA | 1031 | TACCCCAG GGCTAGCTACAACGA TCTGATGT | 5828 |
| 4173 | GAACUGGG G UAAGGACC | 1032 | GGTCCTTA GGCTAGCTACAACGA CCCAGTTC | 5829 |
| 4179 | GGGUAAGG A CCAUCACC | 1033 | GGTCATGG GGCTAGCTACAACGA CCTTACCC | 5830 |
| 4182 | UAAGGACC A UCACCACG | 1034 | CGTGGTGA GGCTAGCTACAACGA GGTCCTTA | 5831 |
| 4185 | GGACCAUC A CCACGGGC | 1035 | GCCCGTGG GGCTAGCTACAACGA GATGGTCC | 5832 |
| 4188 | CCAUCACC A CGGGCGCC | 1036 | GGCGCCCG GGCTAGCTACAACGA GGTGATGG | 5833 |
| 4192 | CACCACGG G CGCCCCGA | 1037 | TGGGGGCG GGCTAGCTACAACGA CCGTGGTG | 5834 |
| 4194 | CCACGGGC G CCCCCAUC | 1038 | CATGGGGG GGCTAGCTACAACGA GCCCGTGG | 5835 |
| 4200 | GCGCCCCC A UCACGUAC | 1039 | GTACGTGA GGCTAGCTACAACGA GGGGGCGC | 5836 |
| 4203 | CCCCCAUC A CGUACUCC | 1040 | GGAGTACG GGCTAGCTACAACGA GATGGGGG | 5837 |
| 4205 | CCCAUCAC G UACUCCAC | 1041 | GTGGAGTA GGCTAGCTACAACGA GTGATGGG | 5838 |
| 4207 | CAUCACGU A CUCCACCU | 1042 | AGGTGGAG GGCTAGCTACAACGA ACGTCATG | 5839 |
| 4212 | CGUACUCC A CCUAUGGC | 1043 | GCCATAGG GGCTAGCTACAACGA GGAGTACG | 5840 |
| 4216 | CUCCACCU A UGGCAAGU | 1044 | ACTTGCGA GGCTAGCTACAACGA AGGTGGAC | 5841 |
| 4219 | CACCUAUG G CAAGUUCC | 1045 | GGAACTTG GGCTAGCTACAACGA CATAGGTG | 5842 |
| 4223 | UAUGGCAA G UUCCUUGC | 1046 | GCAAGGAA GGCTAGCTACAACGA TTGCCATA | 5843 |
| 4230 | AGUUCCUU G CCGACGGU | 1047 | ACCGTCGG GGCTAGCTACAACGA AAGGAACT | 5844 |
| 4234 | CCUUGCCG A CGGUGGUU | 1048 | AACCACCG GGCTAGCTACAACGA CGGCAAGG | 5845 |
| 4237 | UGCCGACG G UGGUUGCU | 1049 | AGCAACGA GGCTAGCTACAACGA CGTCGGCA | 5846 |
| 4240 | CGACGGUG G UUGCUCUG | 1050 | CAGAGCAA GGCTAGCTACAACGA CACCGTCG | 5847 |
| 4243 | CGGUGGUU G CUCUGGGG | 1051 | CCCCAGAG GGCTAGCTACAACGA AACCACCG | 5848 |
| 4252 | CUCUGGGG G CGCCUAUG | 1052 | CATAGGCG GGCTAGCTACAACGA CCCCAGAG | 5849 |
| 4254 | CUGGGGGC G CCUAUGAC | 1053 | GTCATAGG GGCTAGCTACAACGA GCCCCCAG | 5850 |
| 4258 | GGGCGCCU A UGACAUCA | 1054 | TGATGTCA GGCTAGCTACAACGA AGGCGCCC | 5851 |
| 4261 | CGCCUAUG A CAUCAUAA | 1055 | TTATGATG GGCTAGCTACAACGA CATAGGCG | 5852 |
| 4263 | CCUAUGAC A UCAUAAUG | 1056 | CATTATGA GGCTAGCTACAACGA GTCATAGG | 5853 |
| 4266 | AUGACAUC A UAAUCUGU | 1057 | ACACATTA GGCTAGCTACAACGA GATGTCAT | 5854 |
| 4269 | ACAUCAUA A UGUGUGAU | 1058 | ATCACACA GGCTAGCTACAACGA TATGATGT | 5855 |
| 4271 | AUCAUAAU G UCUGAUGA | 1059 | TCATCACA GGCTAGCTACAACGA ATTATGAT | 5856 |
| 4273 | CAUAAUGU G UGAUGAGU | 1060 | ACTCATCA GGCTAGCTACAACGA ACATTATG | 5857 |
| 4276 | AAUGUGUG A UGAGUGCC | 1061 | GGCACTCA GGCTAGCTACAACGA CACACATT | 5858 |
| 4280 | UGUGAUGA G UGCCACUC | 1062 | GAGTGGCA GGCTAGCTACAACGA TCATCACA | 5859 |
| 4282 | UGAUCAGU G CCACUCAA | 1063 | TTGACTGG GGCTAGCTACAACGA ACTCATCA | 5860 |
| 4285 | UCAGUGCC A CUCAAUUG | 1064 | CAATTGAG GGCTAGCTACAACGA GGCACTCA | 5861 |
| 4290 | UCCACUCA A UUGACUCG | 1065 | CGAGTCAA GGCTAGCTACAACGA TGAGTCCC | 5862 |
| 4294 | CUCAAUUG A CUCGACUU | 1066 | AAGTCGAG GGCTAGCTACAACGA CAATTGAG | 5863 |
| 4299 | UUGACUCG A CUUCCAUU | 1067 | AATGGAAG GGCTAGCTACAACGA CCAGTCAA | 5864 |
| 4305 | CGACUUCC A UUUUGGGC | 1068 | GCCCAAAA GGCTAGCTACAACGA CGAAGTCG | 5865 |
| 4312 | CAUUUUGG G CAUCGGCA | 1069 | TGCCGATG GGCTAGCTACAACGA CCAAAATG | 5866 |
| 4314 | UUUUGGGC A UCGGCACA | 1070 | TGTGCCGA GGCTAGCTACAACGA GCCCAAAA | 5867 |
| 4318 | GGGCAUCG G CACAGUCC | 1071 | GGACTGTG GGCTAGCTACAACGA CGATGCCC | 5868 |
| 4320 | GCAUCGGC A CAGUCCUG | 1072 | CAGGACTC GGCTAGCTACAACGA GCCGATGC | 5869 |
| 4323 | UCGGCACA G UCCUGGAC | 1073 | GTCCAGGA GGCTAGCTACAACGA TGTCCCGA | 5870 |
| 4330 | AGUCCUGG A CCAAGCCC | 1074 | CCGCTTGG GGCTAGCTACAACGA CCAGGACT | 5871 |
| 4335 | UGGACCAA G CGGAGACG | 1075 | CGTCTCCG GGCTAGCTACAACGA TTGGTCCA | 5872 |
| 4341 | AAGCGGAG A CGGCUGGA | 1076 | TCCAGCCG GGCTAGCTACAACGA CTCCGCTT | 5873 |
| 4344 | CGGAGACG G CUGGAGCG | 1077 | CGCTCCAG GGCTAGCTACAACGA CGTCTCCG | 5874 |
| 4350 | CCGCUGCA G CGCGGCUC | 1078 | GAGCCGCG GGCTAGCTACAACGA TCCAGCCC | 5875 |
| 4352 | GCUGCAGC G CGGCUCGU | 1079 | ACGAGCCG GGCTAGCTACAACGA GCTCCAGC | 5876 |
| 4355 | GGAGCGCG G CUCGUCGU | 1080 | ACGACGAG GGCTAGCTACAACGA CGCGCTCC | 5877 |
| 4359 | CGCGGCUC G UCGUGCUC | 1081 | GAGCACGA GGCTAGCTACAACGA GAGCCGCG | 5878 |
| 4362 | GGCUCGUC G UGCUCGCC | 1082 | GGCGAGCA GGCTAGCTACAACGA GACGAGCC | 5879 |
| 4364 | CUCGUCGU G CUCGCCAC | 1083 | GTGGCGAG GGCTAGCTACAACGA ACGACGAG | 5880 |
| 4368 | UCGUGCUC G CCACCGCU | 1084 | AGCGGTGG GGCTAGCTACAACGA GAGGACCA | 5881 |
| 4371 | UGCUCGCC A CCGCUACG | 1085 | CGTAGCGG GGCTAGCTACAACGA GGCGAGCA | 5882 |
| 4374 | UCGCCACC G CUACGCCU | 1086 | AGGCGTAG GGCTAGCTACAACGA GGTGGCGA | 5883 |
| 4377 | CCACCGCU A CGCCUCCG | 1087 | CGGAGGCG GGCTAGCTACAACGA AGCGGTGG | 5884 |
| 4379 | ACCGCUAC G CCUCCGGG | 1088 | CCCGGAGG GGCTAGCTACAACGA GTAGCGGT | 5885 |
| 4388 | CCUCCGGG A UCGGUCAC | 1089 | GTGACCGA GGCTAGCTACAACGA CCCGGAGG | 5886 |
| 4392 | CGGGAUCG G UCACCGUG | 1090 | CACGGTGA GGCTAGCTACAACGA CGATCCCG | 5887 |
| 4395 | GAUCGGUC A CCGUGCCA | 1091 | TGGCACGG GGCTAGCTACAACGA GACCGATC | 5888 |
| 4398 | CGGUCACC G UGCCACAU | 1092 | ATGTGGCA GGCTAGCTACAACGA GGTGACCG | 5889 |
| 4400 | GUCACCCU G CCACAUCC | 1093 | GGATGTGG GGCTAGCTACAACGA ACGGTGAC | 5890 |
| 4403 | ACCGUGCC A CAUCCCAA | 1094 | TTGGGATG GGCTAGCTACAACGA GGCACGGT | 5891 |
| 4405 | CGUGCCAC A UCCCAACA | 1095 | TGTTGGGA GGCTAGCTACAACGA GTGGCACG | 5892 |
| 4411 | ACAUCCCA A CAUCGAGG | 1096 | CCTCGATG GGCTAGCTACAACGA TGGGATGT | 5893 |
| 4413 | AUCCCAAC A UCGAGGAG | 1097 | CTCCTCGA GGCTAGCTACAACGA GTTGGGAT | 5894 |
| 4422 | UCCAGGAG A UAGCCUUC | 1098 | CAACGCTA GGCTAGCTACAACGA CTCCTCGA | 5895 |
| 4425 | AGGAGAUA G CCUUGUCC | 1099 | GGACAAGG GGCTAGCTACAACGA TATCTCCT | 5896 |
| 4430 | AUAGCCUU G UCCAACAC | 1100 | GTGTTGGA GGCTAGCTACAACGA AAGGCTAT | 5897 |
| 4435 | CUUGUCCA A CACCGGAG | 1101 | CTCCGGTG GGCTAGCTACAACGA TGGACAAG | 5898 |
| 4437 | UGUCCAAC A CCGGAGAG | 1102 | CTCTCCGG GGCTAGCTACAACGA GTTGGACA | 5899 |
| 4446 | CCGGAGAG A UCCCCUUC | 1103 | GAAGGGGA GGCTAGCTACAACGA CTCTCCGG | 5900 |
| 4456 | CCCCUUCU A UGGCAAAG | 1104 | CTTTGCGA GGCTAGCTACAACGA ACAAGGCG | 5901 |
| 4459 | CUUCUAUC G CAAAGCGA | 1105 | TCCCTTTG GGCTAGCTACAACGA CATAGAAG | 5902 |
| 4464 | AUCCCAAA G CCAUCCCC | 1106 | GGGGATGG GGCTAGCTACAACGA TTTGCCAT | 5903 |
| 4467 | CCAAAGCC A UCCCCAUC | 1107 | CATCGCGA GGCTAGCTACAACGA GGCTTTCC | 5904 |
| 4473 | CCAUCCCC A UCGAGACC | 1108 | GGTCTCGA GGCTAGCTACAACGA GGGCATCC | 5905 |
| 4479 | CCAUCGAG A CCAUCAAA | 1109 | TTTGATGC GGCTAGCTACAACGA CTCGATGG | 5906 |
| 4482 | UCGAGACC A UCAAAGGG | 1110 | CCCTTTGA GGCTAGCTACAACGA GGTCTCGA | 5907 |
| 4496 | GGGCCCAC G CAUCUCAU | 1111 | ATCAGATG GGCTAGCTACAACGA CTCCCCCC | 5908 |
| 4498 | GGCGACGC A UCUCAUCU | 1112 | AGATCACA GGCTAGCTACAACGA GCCTCCCC | 5909 |
| 4503 | GCCAUCUC A UCUUCUCC | 1113 | GCAGAACA GGCTAGCTACAACGA GAGATGCC | 5910 |
| 4510 | CAUCUUCU G CCAUUCCA | 1114 | TCCAATGG GGCTAGCTACAACGA AGAAGATG | 5911 |
| 4513 | CUUCUGCC A UUCCAACA | 1115 | TCTTGGAA GGCTAGCTACAACGA GGCAGAAG | 5912 |
| 4526 | AAGAAGAA A UGUCACGA | 1116 | TCGTCACA GGCTAGCTACAACGA TTCTTCTT | 5913 |
| 4528 | CAAGAAAU G UGACGACC | 1117 | CCTCGTCA CCCTAGCTACAACGA ATTTCTTC | 5914 |
| 4531 | CAAAUGUC A CGAGCUCG | 1118 | CGAGCTCC CCCTAGCTACAACGA CACATTTC | 5915 |
| 4535 | UGUGACGA G CUCGCUGC | 1119 | CCAGCCAC GGCTAGCTACAACGA TCCTCACA | 5916 |
| 4539 | ACCACCUC G CUGCAAAG | 1120 | CTTTGCAC GGCTAGCTACAACGA CACCTCGT | 5917 |
| 4542 | ACCUCCCU G CAAAGCUC | 1121 | CACCTTTC GGCTAGCTACAACGA ACCCACCT | 5918 |
| 4547 | GCUGCAAA G CUGUCGGG | 1122 | CCCCACAC GGCTAGCTACAACGA TTTCCAGC | 5919 |
| 4550 | GCAAAGCU G UCGGCCCU | 1123 | AGCCCCGA GGCTAGCTACAACGA AGCTTTCC | 5920 |
| 4555 | GCUGUCCG G CCUCGCAC | 1124 | CTCCGACG GGCTAGCTACAACGA CCGACACC | 5921 |
| 4562 | CGCCUCGG A CUUAACCC | 1125 | GCGTTAAG GGCTAGCTACAACGA CCCACGCC | 5922 |
| 4567 | CGGACUUA A CGCUCUAG | 1126 | CTACAGCC CCCTAGCTACAACGA TAAGTCCG | 5923 |
| 4569 | GACUUAAC G CUCUACCC | 1127 | CCCTACAC CCCTAGCTACAACGA CTTAACTC | 5924 |
| 4572 | UUAACCCU G UACCCUAU | 1128 | ATACCCTA CCCTAGCTACAACGA ACCCTTAA | 5925 |
| 4575 | ACCCUCUA G CCUAUUAC | 1129 | CTAATACC CCCTAGCTACAACGA TACACCCT | 5926 |
| 4577 | CCUCUACC G UAUUACCC | 1130 | CGCTAATA CCCTAGCTACAACGA CCTACACC | 5927 |
| 4579 | UCUACCCU A UUACCCCC | 1131 | CCCCCTAA CCCTAGCTACAACGA ACCCTACA | 5928 |
| 4582 | ACCCUAUU A CCCCCCUC | 1132 | CACCCCCC GGCTAGCTACAACGA AATACCCT | 5929 |
| 4588 | UUACCCCG G UCUCCACC | 1133 | CCTCCACA CCCTAGCTACAACGA CCCCCTAA | 5930 |
| 4594 | CCCUCUCC A CGUCUCCC | 1134 | CCGACACC GGCTAGCTACAACGA CCACACCC | 5931 |
| 4596 | CUCUCCAC G UCUCCGUC | 1135 | GACCCACA GGCTAGCTACAACGA CTCGACAC | 5932 |
| 4598 | CUCCACCU G UCCCUCAU | 1136 | ATCACCGA CCCTAGCTACAACGA ACCTCCAC | 5933 |
| 4602 | ACGUGUCC G UCAUACCG | 1137 | CGGTATGA GGCTAGCTACAACGA GGACACGT | 5934 |
| 4605 | UGUCCGUC A UACCGGCC | 1138 | GGCCGGTA GGCTAGCTACAACGA GACGGACA | 5935 |
| 4607 | UCCGUCAU A CCGGCCAG | 1139 | CTGGCCGG GGCTAGCTACAACGA ATGACGGA | 5936 |
| 4611 | UCAUACCG G CCAGCGGG | 1140 | CCCGCTGG GGCTAGCTACAACGA CGGTATGA | 5937 |
| 4615 | ACCGGCGA G CGGGGACG | 1141 | CGTCCCCG GGCTAGCTACAACGA TGGCCGGT | 5938 |
| 4621 | CAGCGGGG A CGUCGUUC | 1142 | CAACGACG GGCTAGCTACAACGA CCCCGCTG | 5939 |
| 4623 | GCGGGGAC G UCGUUGUC | 1143 | GACAACGA GGCTAGCTACAACGA GTCCCCGC | 5940 |
| 4626 | GGGACGAC G UUGUCGUG | 1144 | CACGACAA GGCTAGCTACAACGA GACGTCCC | 5941 |
| 4629 | ACGUCGUU G UCGUGGCA | 1145 | TGCCACGA GGCTAGCTACAACGA AACGACGT | 5942 |
| 4632 | UCGUUGUC G UGGCAACA | 1146 | TGTTGCGA GGCTAGCTACAACGA GACAACGA | 5943 |
| 4635 | UUGUCGUG G CAACAGAC | 1147 | GTCTGTTG GGCTAGCTACAACGA CACGACAA | 5944 |
| 4638 | UCGUGGCA A CAGACGCU | 1148 | AGCGTCTG GGCTAGCTACAACGA TGCCACGA | 5945 |
| 4642 | GGCAACAG A CGCUCUAA | 1149 | TTAGAGCG GGCTAGCTACAACGA CTGTTGCC | 5946 |
| 4644 | CAACAGAC G CUCUAAUG | 1150 | CATTAGAG GGCTAGCTACAACGA GTCTGTTG | 5947 |
| 4650 | ACGCUCUA A UGACGGGC | 1151 | GCCCGTCA GGCTAGCTACAACGA TAGAGCGT | 5948 |
| 4653 | CUCUAAUG A CGGGCUAU | 1152 | ATAGCCCG GGCTAGCTACAACGA CATTAGAG | 5949 |
| 4657 | AAUGACGG G CUAUACCG | 1153 | CGGTATAG GGCTAGCTACAACGA CCGTCATT | 5950 |
| 4660 | GACGGGCU A UACCGGCG | 1154 | CGCCGGTA GGCTAGCTACAACGA AGCCCGTC | 5951 |
| 4662 | CGGGCUAU A CCGGCGAU | 1155 | ATCGCCGG GGCTAGCTACAACGA ATAGCCCG | 5952 |
| 4666 | CUAUACCG G CGAUUUUG | 1156 | CAAAATCG GGCTAGCTACAACGA CGGTATAG | 5953 |
| 4669 | UACCGGCG A UUUUGACU | 1157 | AGTCAAAA GGCTAGCTACAACGA CGCCGGTA | 5954 |
| 4675 | CGAUUUUG A CUCGGUGA | 1158 | TCACCGAG GGCTAGCTACAACGA CAAAATCG | 5955 |
| 4680 | UUGACUCG G UGAUCGAC | 1159 | GTCGATCA GGCTAGCTACAACGA CGAGTCAA | 5956 |
| 4683 | ACUCGGUG A UCGACUGU | 1160 | ACAGTCGA GGCTAGCTACAACGA CACCGAGT | 5957 |
| 4687 | GGUGAUCG A CUGUAAUA | 1161 | TATTACAG GGCTAGCTACAACGA CGATCACC | 5958 |
| 4690 | GAUCGACU G UAAUACAU | 1162 | ATGTATTA GGCTAGCTACAACGA AGTCGATC | 5959 |
| 4693 | CGACUGUA A UACAUGUG | 1163 | CACATGTA GGCTAGCTACAACGA TACAGTCG | 5960 |
| 4695 | ACUGUAAU A CAUGUGUC | 1164 | GACACATG GGCTAGCTACAACGA ATTACAGT | 5961 |
| 4697 | UGUAAUAC A UGUGUCAC | 1165 | GTGACACA GGCTAGCTACAACGA GTATTACA | 5962 |
| 4699 | UAAUACAU G UGUCACCC | 1166 | GGGTGACA GGCTAGCTACAACGA ATGTATTA | 5963 |
| 4701 | AUACAUGU G UCACCCAA | 1167 | TTGGGTGA GGCTAGCTACAACGA ACATGTAT | 5964 |
| 4704 | CAUGUGUC A CCCAAACA | 1168 | TGTTTGGG GGCTAGCTACAACGA GACACATG | 5965 |
| 4710 | UCACCCAA A CAGUCGAC | 1169 | GTCGACTG GGCTAGCTACAACGA TTGGGTGA | 5966 |
| 4713 | CCCAAACA G UCGACUUC | 1170 | GAAGTCGA GGCTAGCTACAACGA TGTTTGGG | 5967 |
| 4717 | AACAGUCG A CUUCAGCU | 1171 | AGCTGAAG GGCTAGCTACAACGA CGACTGTT | 5968 |
| 4723 | CGACUUCA G CUUGGACC | 1172 | GGTCCAAG GGCTAGCTACAACGA TGAAGTCG | 5969 |
| 4729 | CAGCUUGG A CCCUACCU | 1173 | AGGTAGGG GGCTAGCTACAACGA CCAAGCTG | 5970 |
| 4734 | UGGACCCU A CCUUCACC | 1174 | GGTGAAGG GGCTAGCTACAACGA AGGGTCCA | 5971 |
| 4740 | CUACCUUC A CCAUUGAG | 1175 | CTCAATGG GGCTAGCTACAACGA GAAGGTAG | 5972 |
| 4743 | CCUUCACC A UUGAGACG | 1176 | CGTCTCAA GGCTAGCTACAACGA GGTGAAGG | 5973 |
| 4749 | CCAUUGAG A CGACGACC | 1177 | GGTCGTCG GGCTAGCTACAACGA CTCAATGG | 5974 |
| 4752 | UUGAGACG A CGACCGUG | 1178 | CACGGTCG GGCTAGCTACAACGA CGTCTCAA | 5975 |
| 4755 | AGACGACG A CCGUGCCC | 1179 | GGCCACGG GGCTAGCTACAACGA CGTCGTCT | 5976 |
| 4758 | CGACGACC G UGCCCCAA | 1180 | TTGGGGCA GGCTAGCTACAACGA GGTCGTCG | 5977 |
| 4760 | ACGACCGU G CCCCAAGA | 1181 | TCTTGGGG GGCTAGCTACAACGA ACGGTCGT | 5978 |
| 4768 | GCCCCAAG A CGCAGUGU | 1182 | ACACTGCG GGCTAGCTACAACGA CTTGGGGC | 5979 |
| 4770 | CCCAAGAC G CAGUGUCC | 1183 | GGACACTG GGCTAGCTACAACGA GTCTTGGG | 5980 |
| 4773 | AAGACGCA G UGUCCCGC | 1184 | GCGGGACA GGCTAGCTACAACGA TGCGTCTT | 5981 |
| 4775 | GACGCAGU G UCCCGCUC | 1185 | GAGCGGGA GGCTAGCTACAACGA ACTGCGTC | 5982 |
| 4780 | AGUGUCCC G CUCGCAGA | 1186 | TCTGCGAG GGCTAGCTACAACGA GGGACACT | 5983 |
| 4784 | UCCCGCUC G CAGAGGCG | 1187 | CGCCTCTG GGCTAGCTACAACGA CAGCGGGA | 5984 |
| 4790 | UCGCAGAG G CGAGGUAG | 1188 | CTACCTCG GGCTAGCTACAACGA CTCTGCGA | 5985 |
| 4795 | GAGGCGAG G UAGGACCG | 1189 | CGGTCCTA GGCTAGCTACAACGA CTCGCCTC | 5986 |
| 4800 | CAGGUACC A CCGGUAGG | 1190 | CCTACCGC GGCTAGCTACAACGA CCTACCTC | 5987 |
| 4804 | UAGGACCG G UAGGGGCA | 1191 | TGCCCCTA GGCTAGCTACAACGA CGGTCCTA | 5988 |
| 4810 | CGGUAGGG G CAGGAGAG | 1192 | CTCTCCTG GGCTAGCTACAACGA CCCTACCG | 5989 |
| 4819 | CAGGAGAG G CAGAGACA | 1193 | TGTATATG GGCTAGCTACAACGA CTCTCCTG | 5990 |
| 4821 | GGAGAGGC A UAUACAGG | 1194 | CCTGTATA GGCTAGCTACAACGA GCCTCTCC | 5991 |
| 4823 | AGAGGCAU A UACAGGUU | 1195 | AACCTGTA GGCTAGCTACAACGA ATGCCTCT | 5992 |
| 4825 | AGGCAUAU A CAGGUUUG | 1196 | CAAACCTG GGCTAGCTACAACGA ATATGCCT | 5993 |
| 4829 | AUAUACAG G UUUCUGAC | 1197 | CTCACAAA QCCTAGCTACAACGA CTGTATAT | 5994 |
| 4833 | ACAGGUUU G UGACUCCA | 1198 | TGGAGTCA GGCTAGCTACAACGA AAACCTGT | 5995 |
| 4836 | GGUUUGUG A CUCCAGGA | 1199 | TCCTGGAG GGCTAGCTACAACGA CACAAACC | 5996 |
| 4847 | CCAGGAGA G CGGCCUUC | 1200 | GAAGGCCG GGCTAGCTACAACGA TCTCCTCG | 5997 |
| 4850 | GGAGAGCG G CCUUCGGG | 1201 | CCCGAAGG GGCTAGCTACAACGA CGCTCTCC | 5998 |
| 4858 | GCCUUCGG G CAUGUUCG | 1202 | CGAACATG GGCTAGCTACAACGA CCGAAGGC | 5999 |
| 4860 | CUUCGGGC A UGUUCGAC | 1203 | GTCGAACA GGCTAGCTACAACGA GCCCGAAG | 6000 |
| 4862 | UCGGGCAU G UUCGACUC | 1204 | GAGTCGAA GGCTAGCTACAACGA ATGCCCGA | 6001 |
| 4867 | CAUGUUCG A CUCCUCGG | 1205 | CCGAGGAG GGCTAGCTACAACGA CGAACATG | 6002 |
| 4875 | ACUCCUCG G UCCUGUGU | 1206 | ACACAGGA GGCTAGCTACAACGA CGAGCAGT | 6003 |
| 4880 | UCGGUCCU G UGUGAGUG | 1207 | CACTCACA GGCTAGCTACAACGA AGGACCGA | 6004 |
| 4882 | GGUCCUGU G UGAGUGCU | 1208 | AGCACTCA GGCTAGCTACAACGA ACAGGACC | 6005 |
| 4886 | CUGUGUGA G UGCUAUGA | 1209 | TCATAGCA GGCTAGCTACAACGA TCACACAG | 6006 |
| 4888 | GUGUGAGU G CUAUGACG | 1210 | CGTCATAG GGCTAGCTACAACGA ACTCACAC | 6007 |
| 4891 | UGAGUGCU A UGACGCGG | 1211 | CCGCGTCA GGCTAGCTACAACGA AGCACTCA | 6008 |
| 4894 | GUGCUAUG A CGCGGGAU | 1212 | ATCCCGCG GGCTAGCTACAACGA CATACCAC | 6009 |
| 4896 | GCUAUGAC G CGGGAUGU | 1213 | ACATCCCG GGCTAGCTACAACGA GTCATAGC | 6010 |
| 4901 | GACGCGGG A UGUGCUUG | 1214 | CAAGCACA GGCTAGCTACAACGA CCCGCGTC | 6011 |
| 4903 | CGCGGGAU G UGCUUGGU | 1215 | ACCAAGCA GGCTAGCTACAACGA ATCCCGCG | 6012 |
| 4905 | CGGGAUGU G CUUGGUAC | 1216 | GTACCAAG GGCTAGCTACAACGA ACATCCCG | 6013 |
| 4910 | UGUGCUUG G UACCACCU | 1217 | AGCTCGTA GGCTAGCTACAACGA CAAGCACA | 6014 |
| 4912 | UGCUUGGU A CGAGCUCA | 1218 | TGAGCTCG GGCTAGCTACAACGA ACCAAGCA | 6015 |
| 4916 | UGGUACGA G CUCACGCC | 1219 | GGCGTGAG GGCTAGCTACAACGA TCCTACCA | 6016 |
| 4920 | ACCACCUC A CGCCCGCC | 1220 | GGCGGGCG GGCTAGCTACAACGA GAGCTCGT | 6017 |
| 4922 | CACCUCAC G CCCGCCGA | 1221 | TCCGCCCC GGCTAGCTACAACGA CTGAGCTC | 6018 |
| 4926 | UCACGCCC G CCGAGACC | 1222 | GCTCTCGG GGCTAGCTACAACGA GGGCGTGA | 6019 |
| 4932 | CCCCCGAG A CCUCCCUU | 1223 | AACGGAGC GGCTAGCTACAACGA CTCCGCGC | 6020 |
| 4938 | AGACCUCC G UUACGUUG | 1224 | CAACCTAA GGCTAGCTACAACGA GGACCTCT | 6021 |
| 4943 | UCCGUUAG G UUGCGGGC | 1225 | GCCCGCAA GGCTAGCTACAACGA CTAACGCA | 6022 |
| 4946 | GUUAGCUU G CCGGCUUA | 1226 | TAAGCCCC GGCTAGCTACAACGA AACCTAAC | 6023 |
| 4950 | CGUUGCGC G CUUACCUA | 1227 | TAGCTAAG GGCTAGCTACAACGA CCGCAACC | 6024 |
| 4954 | CCGGCCUU A CCUAAAUA | 1228 | TATTTACG GGCTAGCTACAACGA AAGCCCGC | 6025 |
| 4960 | UUACCUAA A UACACCAG | 1229 | CTGGTGTA GGCTAGCTACAACGA TTAGGTAA | 6026 |
| 4962 | ACCUAAAU A CACCACGG | 1230 | CCCTGCTC GGCTAGCTACAACGA ATTTAGCT | 6027 |
| 4964 | CUAAAUAC A CCAGCCUU | 1231 | AACCCTGG GGCTAGCTACAACGA CTATTTAG | 6028 |
| 4970 | ACACCAGG G UUCCCCUU | 1232 | AACGGCAA GGCTAGCTACAACGA CCTCCTGT | 6029 |
| 4973 | CCACGCUU G CCCUUCUC | 1233 | CAGAAGGG GGCTAGCTACAACGA AACCCTCG | 6030 |
| 4981 | GCCCUUCU G CCACCACC | 1234 | GGTCCTCG GGCTAGCTACAACGA AGAAGGGC | 6031 |
| 4987 | CUGCCAGC A CCAUCUGC | 1235 | CCAGATGC GGCTAGCTACAACGA CCTCCCAG | 6032 |
| 4990 | CCACGACC A UCUCGAGU | 1236 | ACTCCAGA GGCTAGCTACAACGA CGTCCTCG | 6033 |
| 4997 | CAUCUCGA G UUCUGGGA | 1237 | TCCCAGAA GGCTAGCTACAACGA TCCACATC | 6034 |
| 5008 | CUCGGAGC G UCUCUUCA | 1238 | TGAACACA GGCTAGCTACAACGA CCTCCCAG | 6035 |
| 5010 | CCCACCCU G UCUUCACA | 1239 | TCTCAAGA GGCTAGCTACAACGA ACCCTCCC | 6036 |
| 5016 | CUGUCUUC A CACCCCUC | 1240 | CACCCCTC GGCTAGCTACAACGA CAACACAC | 6037 |
| 5020 | CUUCACAG G CCUCACCC | 1241 | CCCTCACC GGCTAGCTACAACGA CTCTCAAC | 6038 |
| 5025 | CACCCCUC A CCCACAUA | 1242 | TATGTCCC GGCTAGCTACAACGA CACCCCTC | 6039 |
| 5029 | CCUCACCC A CAUACAUC | 1243 | CATCTATG GGCTAGCTACAACGA CCCTCACC | 6040 |
| 5031 | UCACCCAC A UAGAUGCC | 1244 | CCCATCTA GGCTAGCTACAACGA GTGCCTGA | 6041 |
| 5035 | CCACAUAC A UCCCCACU | 1245 | AGTCGGCA GGCTAGCTACAACGA CTATGTGG | 6042 |
| 5037 | ACAUACAU G CCCACUUC | 1246 | CAACTCCC GGCTAGCTACAACGA ATCTATCT | 6043 |
| 5041 | ACAUCCCC A CUUCUUGU | 1247 | ACAACAAC GGCTAGCTACAACGA CCCCATCT | 6044 |
| 5048 | CACUUCUU G UCCCACAC | 1248 | CTCTCCGA GGCTAGCTACAACGA AACAACTC | 6045 |
| 5055 | UCUCCCAG A CCAACCAC | 1249 | CTCCTTCC GGCTAGCTACAACGA CTCCGACA | 6046 |
| 5060 | CACACCAA G CACCCACC | 1250 | CCTCCCTC GGCTAGCTACAACGA TTGCTCTC | 6047 |
| 5064 | CCAAGCAG G CAGGAGAA | 1251 | TTCTCCTG GGCTAGCTACAACGA CTGCTTGG | 6048 |
| 5074 | AGGAGAAA A CCUCCCCU | 1252 | AGGGGAGG GGCTAGCTACAACGA TTTCTCCT | 6049 |
| 5083 | CCUCCCCU A CCUGGUAG | 1253 | CTACCAGG GGCTAGCTACAACGA AGGGGAGG | 6050 |
| 5088 | CCUACCUG G UAGCAUAC | 1254 | GTATGCTA GGCTAGCTACAACGA CAGGTAGG | 6051 |
| 5091 | ACCUGGGA G CAUACCAA | 1255 | TTGGTATG GGCTAGCTACAACGA TACCAGGT | 6052 |
| 5093 | CUGGUAGC A UACCAAGC | 1256 | GCTTGGTA GGCTAGCTACAACGA GCTACCAG | 6053 |
| 5095 | GGUAGCAU A CCAAGCGA | 1257 | TGGCTTGG GGCTAGCTACAACGA ATGCTACC | 6054 |
| 5100 | CAUACCAA G CCACAGUG | 1258 | CACTGTGG GGCTAGCTACAACGA TTGGTATG | 6055 |
| 5103 | ACCAAGCC A CAGUGUGC | 1259 | GCACACTG GGCTAGCTACAACGA GGCTTGGT | 6056 |
| 5106 | AAGCCACA G UGUGCGCC | 1260 | GGCGCACA GGCTAGCTACAACGA TGTCGCTT | 6057 |
| 5108 | GCCACAGU G UCCUCCAC | 1261 | CTGCCCGA GGCTAGCTACAACGA ACTGTGGC | 6058 |
| 5110 | CACAGUGU G CGCCAGCG | 1262 | CCCTGGCG GGCTAGCTACAACGA ACACTGTG | 6059 |
| 5112 | CAGUGUGC G CCAGGGCU | 1263 | AGCCCTGG GGCTAGCTACAACGA CCACACTG | 6060 |
| 5118 | GCCCCACC G CUCACCCU | 1264 | AGCCTGAG CCCTAGCTACAACGA CCTCGCCC | 6061 |
| 5124 | CCGCUCAG G CUCCACCC | 1265 | CCCTCGAG GGCTAGCTACAACGA CTCACCCC | 6062 |
| 5129 | CAGGCUCC A CCCCCAUC | 1266 | GATCCGCC GGCTAGCTACAACGA GCAGCCTG | 6063 |
| 5135 | CCACCCCC A UCGUGGGA | 1267 | TCCCACGA GGCTAGCTACAACGA CCCGGTGG | 6064 |
| 5138 | CCCCCAUC G UCCCAUCA | 1268 | TCATCCGA GGCTAGCTACAACGA CATGGGGG | 6065 |
| 5143 | AUCCUCGC A UCAAAUCU | 1269 | ACATTTCA CCCTAGCTACAACGA CCCACGAT | 6066 |
| 5148 | GGGAUCAA A UGUGCAAC | 1270 | CTTCCACA GGCTAGCTACAACGA TTGATCCC | 6067 |
| 5150 | GAUCAAAU G UGGAACUG | 1271 | CACTTCGA GGCTAGCTACAACGA ATTTGATC | 6068 |
| 5156 | AUCUGGAA G UCUCUCAC | 1272 | GTCACACA GGCTAGCTACAACGA TTCCACAT | 6069 |
| 5158 | GUGGAAGU G UCUCACAC | 1273 | CTGTGAGA GGCTAGCTACAACGA ACTTCCAC | 6070 |
| 5163 | AGUGUCUC A CACGGCUA | 1274 | TAGCCCTG GGCTAGCTACAACGA CAGACACT | 6071 |
| 5165 | UGUCUCAC A CGCCUAAA | 1275 | TTTAGCCG GGCTAGCTACAACGA GTGAGACA | 6072 |
| 5168 | CUCACACG G CUAAAGCC | 1276 | GGCTTTAG GGCTAGCTACAACGA CGTGTGAG | 6073 |
| 5174 | CGGCUAAA G CCUACGCU | 1277 | AGCGTAGG GGCTAGCTACAACGA TTTAGCCG | 6074 |
| 5178 | UAAACCCU A CGCUACAC | 1278 | CTGTAGCG GGCTAGCTACAACGA AGCCTTTA | 6075 |
| 5180 | AAGCCUAC G CUACACCG | 1279 | CCCTGTAC GGCTAGCTACAACGA CTACCCTT | 6076 |
| 5183 | CCUACGCU A CACGGGCC | 1280 | GCCCCCTC GGCTAGCTACAACGA AGCGTACC | 6077 |
| 5185 | UACCCUAC A CGCCCCAA | 1281 | TTGCCCCC GGCTAGCTACAACGA CTACCGTA | 6078 |
| 5189 | CUACACCG G CCAACACC | 1282 | CGTGTTGG GGCTAGCTACAACGA CCCTGTAC | 6079 |
| 5193 | ACCCGCCA A CACCCCUG | 1283 | CACGGGTG GGCTAGCTACAACGA TCCCCCGT | 6080 |
| 5195 | GGCCCAAC A CCCCUGCU | 1284 | ACCACCCC GGCTAGCTACAACGA GTTCCCCC | 6081 |
| 5201 | ACACCCCU G CUCUAUAC | 1285 | CTATACAC CCCTAGCTACAACGA ACCCCTCT | 6082 |
| 5204 | CCCCUCCU G UAUACGCU | 1286 | ACCCTATA CCCTAGCTACAACGA ACCACCCC | 6083 |
| 5206 | CCUCCUCU A UACCCUAC | 1287 | CTACCCTA CCCTAGCTACAACGA ACACCACC | 6084 |
| 5210 | CUCUAUAC G CUACCACC | 1288 | CCTCCTAC CCCTAGCTACAACGA CTATACAC | 6085 |
| 5217 | CCCUACGA G CCCUCCAA | 1289 | TTCCACCC CCCTAGCTACAACGA TCCTACCC | 6086 |
| 5220 | UACCAGCC G UCCAAAAU | 1290 | ATTTTGGA GGCTAGCTACAACGA CCCTCCTA | 6087 |
| 5227 | CCUCCAAA A UCAUCUCA | 1291 | TCACATCA GGCTAGCTACAACGA TTTGCACC | 6088 |
| 5230 | CCAAAAUC A UCUCACCC | 1292 | CCGTGACA GGCTAGCTACAACGA CATTTTCC | 6089 |
| 5232 | AAAAUCAU G UCACCCUC | 1293 | CACCCTCA CCCTAGCTACAACGA ATCATTTT | 6090 |
| 5235 | AUCAUCUC A CCCUCACA | 1294 | TCTCACCC CCCTAGCTACAACGA CACATCAT | 6091 |
| 5241 | UCACCCUC A CACACCCC | 1295 | CCGCTCTC CCCTAGCTACAACGA CACCCTCA | 6092 |
| 5243 | ACCCUCAC A CACCCCAU | 1296 | ATCCCCTC GGCTAGCTACAACGA CTCACCCT | 6093 |
| 5245 | CCUCACAC A CCCCAUAA | 1297 | TTATCCCC CCCTAGCTACAACGA CTCTGACG | 6094 |
| 5250 | CACACCCC A UAACCAAA | 1298 | TTTCCTTA CCCTAGCTACAACGA CCCCTCTC | 6095 |
| 5253 | ACCCCAUA A CCAAAUAC | 1299 | CTATTTCC CCCTAGCTACAACGA TATCGCCT | 6096 |
| 5258 | AUAACCAA A UACAUCAU | 1300 | ATCATCTA CCCTAGCTACAACGA TTGCTTAT | 6097 |
| 5260 | AACCAAAU A CAUCAUGA | 1301 | TCATCATC CCCTAGCTACAACGA ATTTCCTT | 6098 |
| 5262 | CCAAAUAC A UCAUGACA | 1302 | TCTCATCA CCCTAGCTACAACGA GTATTTCC | 6099 |
| 5265 | AAUACAUC A UCACAUGC | 1303 | CCATCTCA CCCTAGCTACAACGA GATGTATT | 6100 |
| 5268 | ACAUCAUC A CAUCCAUG | 1304 | CATCCATC CCCTAGCTACAACGA CATCATCT | 6101 |
| 5270 | AUCAUCAC A UGCAUGUC | 1305 | CACATCGA CCCTAGCTACAACGA GTCATGAT | 6102 |
| 5272 | CAUCACAU G CAUCUCGG | 1306 | CCGACATG CCCTAGCTACAACGA ATGTCATG | 6103 |
| 5274 | UCACAUCC A UGUCGGCU | 1307 | ACCCCACA CCCTAGCTACAACGA CCATCTCA | 6104 |
| 5276 | ACAUGCAU G UCGGCUGA | 1308 | TCAGCCGA GGCTAGCTACAACGA ATGCATGT | 6105 |
| 5280 | GCAUGUCG G CUGACCUG | 1309 | CAGGTCAG GGCTAGCTACAACGA CGACATGC | 6106 |
| 5284 | GUCGGCUG A CCUGGAGG | 1310 | CCTCCAGG GGCTAGCTACAACGA CAGCCGAC | 6107 |
| 5292 | ACCUGGAG G UCGUCACC | 1311 | GGTGACGA GGCTAGCTACAACGA CTCCAGGT | 6108 |
| 5295 | UGGAGGUC G UCACCAGC | 1312 | GCTGGTGA GGCTAGCTACAACGA GACCTCCA | 6109 |
| 5298 | AGGUCCUC A CCAGCACC | 1313 | GGTGCTGG GGCTAGCTACAACGA GACGACCT | 6110 |
| 5302 | CGUCACGA G CACCUGGG | 1314 | CCCAGGTG GGCTAGCTACAACGA TGGTGACG | 6111 |
| 5304 | UCACCAGC A CCUGGGUG | 1315 | CACCCAGG GGCTAGCTACAACGA GCTGGTGA | 6112 |
| 5310 | GCACCUGG G UGCUAGUA | 1316 | TACTAGCA GGCTAGCTACAACGA CCAGGTGC | 6113 |
| 5312 | ACCUGGGU G CUAGUAGG | 1317 | CCTACTAG GGCTAGCTACAACGA ACCCAGGT | 6114 |
| 5316 | GGGUGCUA G UAGGUGGC | 1318 | GCCACCTA GGCTAGCTACAACGA TAGCACCC | 6115 |
| 5320 | GCUAGUAG G UGGCGUCC | 1319 | GGACGCGA GGCTAGCTACAACGA CTACTAGC | 6116 |
| 5323 | AGUAGGUG G CGUCCUGG | 1320 | CCAGGACG GGCTAGCTACAACGA CACCTACT | 6117 |
| 5325 | UAGGUGGC G UCCUGGCA | 1321 | TGCCAGGA GGCTAGCTACAACGA GCCACCTA | 6118 |
| 5331 | GCGUCCUG G CAGCUCUG | 1322 | CAGAGCTG GGCTAGCTACAACGA CAGGACGC | 6119 |
| 5334 | UCCUGUCA G CUCUGACC | 1323 | GGTCAGAG GGCTAGCTACAACGA TGCCACGA | 6120 |
| 5340 | CAGCUCUG A CCGCGUAU | 1324 | ATACGCGG GGCTAGCTACAACGA CAGAGCTG | 6121 |
| 5343 | CUCUGACC G CGUAUUGC | 1325 | GCAATACG GGCTAGCTACAACGA GGTCAGAG | 6122 |
| 5345 | CUGACCGC G UAUUGCCU | 1326 | AGGCAATA GGCTAGCTACAACGA GCGGTCAC | 6123 |
| 5347 | GACCGCGU A UUGCCUGA | 1327 | TCAGGCAA GGCTAGCTACAACGA ACGCGGTC | 6124 |
| 5350 | CGCGUAUU G CCUGACGA | 1328 | TCGTCAGG GGCTAGCTACAACGA AATACGCG | 6125 |
| 5355 | AUUGCCUG A CGACAGGC | 1329 | GCCTGTCG GGCTAGCTACAACGA CAGGCAAT | 6126 |
| 5358 | GCCUGACG A CAGGCAGC | 1330 | GCTGCCTG GGCTAGCTACAACGA CGTCAGGC | 6127 |
| 5362 | GACGACAG G CAGCGUGG | 1331 | CCACGCTG GGCTAGCTACAACGA CTGTCGTC | 6128 |
| 5365 | GACAGGCA G CGUGGUCA | 1332 | TGACCACG GGCTAGCTACAACGA TGCCTGTC | 6129 |
| 5367 | CAGCCAGC G UGGUCAUU | 1333 | AATGACGA GGCTAGCTACAACGA CCTGCCTG | 6130 |
| 5370 | GCAGCGUG G UCAUGGUG | 1334 | CACAATGA GGCTAGCTACAACGA CACGCTGC | 6131 |
| 5373 | GCGUGGUC A UUGUGGGC | 1335 | GCCCACAA GGCTAGCTACAACGA GACCACCC | 6132 |
| 5376 | UGGUCAUU G UGGGCAGA | 1336 | TCTGCCGA GGCTAGCTACAACGA AATGACCA | 6133 |
| 5380 | CAUUGUGG G CAGAAUCA | 1337 | TGATTCTG GGCTAGCTACAACGA CCACAATG | 6134 |
| 5385 | UGGGCAGA A UCAUCUUG | 1338 | CAAGATGA GGCTAGCTACAACGA TCTGCCCA | 6135 |
| 5368 | GCAGAAUC A UCUUGUCC | 1339 | GGACAAGA GGCTAGCTACAACGA GATTCTCC | 6136 |
| 5393 | AUCAUCUU G UCCGGGAA | 1340 | TTCCCGGA GGCTAGCTACAACGA AAGATGAT | 6137 |
| 5402 | UCCGGGAA G CCGGCUGU | 1341 | ACAGCCCG GGCTAGCTACAACGA TTCCCGGA | 6138 |
| 5406 | GGAAGCCG G CUGUUAUC | 1342 | GATAACAG GGCTAGCTACAACGA CGCCTTCC | 6139 |
| 5409 | AGCCGGCU G UUAUCCCC | 1343 | GGGGATAA GGCTAGCTACAACGA AGCCGGCT | 6140 |
| 5412 | CGGCUGUU A UCCCCGAC | 1344 | GTCGCCGA GGCTAGCTACAACGA AACAGCCG | 6141 |
| 5419 | UAUCCCCG A CAGGGAGG | 1345 | CCTCCCTG GGCTAGCTACAACGA CGGGGATA | 6142 |
| 5427 | ACAGGGAG G CUCUCUAC | 1346 | GTACAGAG GGCTAGCTACAACGA CTCCCTGT | 6143 |
| 5434 | GGCUCUCU A CCAGGAGU | 1347 | ACTCCTGG GGCTAGCTACAACGA AGAGAGCC | 6144 |
| 5441 | UACCAGGA G UUCGAUCA | 1348 | TCATCCAA GGCTAGCTACAACGA TCCTGCTA | 6145 |
| 5446 | CCACUUCC A UCACAUCC | 1349 | CCATCTCA CCCTAGCTACAACGA CCAACTCC | 6146 |
| 5451 | UCCAUCAG A UCCACCAC | 1350 | CTCCTCGA CCCTAGCTACAACGA CTCATCCA | 6147 |
| 5459 | AUGGACGA G UCUGCCUC | 1351 | CACCCACA GGCTAGCTACAACGA TCCTCCAT | 6148 |
| 5461 | CCACCACU G UGCCUCAC | 1352 | CTGACCGA GGCTAGCTACAACGA ACTCCTCC | 6149 |
| 5463 | ACCAGUCU G CCUCACAC | 1353 | CTCTCACC GGCTAGCTACAACGA ACACTCCT | 6150 |
| 5468 | UCUCCCUC A CACCUCCC | 1354 | GCCACCTG GGCTAGCTACAACGA CAGCCACA | 6151 |
| 5470 | UCCCUCAC A CCUCCCUU | 1355 | AACCCACC GGCTAGCTACAACGA CTCACCCA | 6152 |
| 5479 | CCUCCCUU A CAUCCAAC | 1356 | CTTCCATC CCCTAGCTACAACGA AACCCAGC | 6153 |
| 5481 | UCCCUUAC A UCGAACAG | 1357 | CTGTTCGA GGCTAGCTACAACGA GTAAGGGA | 6154 |
| 5486 | UACAUCCA A CACGCGAU | 1358 | ATCCCCTC GGCTAGCTACAACGA TCCATCTA | 6155 |
| 5493 | AACAGGGG A UCCACCUC | 1359 | GAGCTCGA GGCTAGCTACAACGA CCCCTCTT | 6156 |
| 5495 | CACCCCAU G CACCUCCC | 1360 | CCCACCTC GGCTAGCTACAACGA ATCCCCTG | 6157 |
| 5498 | CCGAUCGA G CUCCCCGA | 1361 | TCCGCCAC GGCTAGCTACAACGA TGCATCCC | 6158 |
| 5502 | UCCACCUC G CCCACCAC | 1362 | CTCCTCCC CCCTAGCTACAACGA CACCTCCA | 6159 |
| 5507 | CUCCCCGA G CAGUUCAA | 1363 | TTGAACTC CCCTAGCTACAACGA TCCGCCAC | 6160 |
| 5510 | GCCCACGA G UUCAACGA | 1364 | TGCTTCAA GGCTAGCTACAACGA TGCTCCCC | 6161 |
| 5516 | CAGUUCAA G CAGAAGGC | 1365 | GCCTTCTG GGCTAGCTACAACGA TTGAACTG | 6162 |
| 5523 | AGCAGAAG G CGCUCGGA | 1366 | TCCGAGCG GGCTAGCTACAACGA CTTCTGCT | 6163 |
| 5525 | CAGAAGGC G CUCGGAUU | 1367 | AATCCGAG GGCTAGCTACAACGA GCCTTCTG | 6164 |
| 5531 | GCGCUCGG A UUGCUGCA | 1368 | TGCAGCAA GGCTAGCTACAACGA CCGAGCGC | 6165 |
| 5534 | CUCCGAUU G CUGCAAAC | 1369 | GTTTGCAG GGCTAGCTACAACGA AATCCGAG | 6166 |
| 5537 | GGAUUGCU G CAAACAGC | 1370 | GCTGTTTG GGCTAGCTACAACGA AGCAATCC | 6167 |
| 5541 | UGCUGCAA A CAGCCACC | 1371 | GGTGGCTG GGCTAGCTACAACGA TTGCAGCA | 6168 |
| 5544 | UGCAAACA G CCACCAAC | 1372 | GTTGGTGG GGCTAGCTACAACGA TGTTTGCA | 6169 |
| 5547 | AAACAGCC A CCAACCAA | 1373 | TTGGTTGG GGCTAGCTACAACGA GGCTGTTT | 6170 |
| 5551 | AGCCACGA A CCAAGCGG | 1374 | CCGCTTGG GGCTAGCTACAACGA TGGTGGCT | 6171 |
| 5556 | CCAACCAA G CGGAGGCU | 1375 | AGCCTCCG GGCTAGCTACAACGA TTGGTTGG | 6172 |
| 5562 | AAGCGGAG G CUGCUGCU | 1376 | AGCAGCAG GGCTAGCTACAACGA CTCCGCTT | 6173 |
| 5565 | CGGAGGCU G CUGCUCCC | 1377 | GGGAGCAG GGCTAGCTACAACGA AGCCTCCG | 6174 |
| 5568 | AGGCUGCU G CUCCCGUG | 1378 | CACGGGAG GGCTAGCTACAACGA AGCAGCCT | 6175 |
| 5574 | CUGCUCCC G UGGUGGAA | 1379 | TTCCACGA GGCTAGCTACAACGA GGGAGCAG | 6176 |
| 5577 | CUCCCGUG G UGGAAUCC | 1380 | GGATTCGA GGCTAGCTACAACGA CACGGGAG | 6177 |
| 5582 | GUGGUGGA A UCCAAGUG | 1381 | CACTTGGA GGCTAGCTACAACGA TCCACCAC | 6178 |
| 5588 | GAAUCCAA G UGGCGAGC | 1382 | GCTCGCGA GGCTAGCTACAACGA TTGGATTC | 6179 |
| 5591 | UCCAAGUG G CGAGCCCU | 1383 | AGGGCTCG GGCTAGCTACAACGA CACTTGGA | 6180 |
| 5595 | AGUGGCGA G CCCUUGAG | 1384 | CTCAAGGG GGCTA0CTACAACGA TCGCCACT | 6181 |
| 5604 | CCCUUGAG G CUUUCUGG | 1385 | CCAGAAAG GGCTAGCTACAACGA CTCAAGGG | 6182 |
| 5613 | CUUUCUGG G CGAAGCAC | 1386 | GTGCTTCG GGCTAGCTACAACGA CCAGAAAG | 6183 |
| 5618 | UGGGCGAA G CACAUGUG | 1387 | CACATGTG GGCTAGCTACAACGA TTCGCCCA | 6184 |
| 5620 | GGCGAAGC A CAUGUGGA | 1388 | TCCACATG GGCTAGCTACAACGA GCTTCGCC | 6185 |
| 5622 | CGAAGCAC A UGUUGAAU | 1389 | ATTCCACA GGCTAGCTACAACGA GTGCTTCG | 6186 |
| 5624 | AAGCACAU G UGGAAUUU | 1390 | AAATTCGA GGCTAGCTACAACGA ATGTGCTT | 6187 |
| 5629 | CAUGUGGA A UUUCAUCA | 1391 | TGATGAAA GGCTAGCTACAACGA TCCACATG | 6188 |
| 5634 | GGAAUUUC A UCAGCGGG | 1392 | CCCGCTGA GGCTAGCTACAACGA GAAATTCC | 6189 |
| 5638 | UUUCAUCA G CGGGAUAC | 1393 | GTATCCCG GGCTAGCTACAACGA TGATGAAA | 6190 |
| 5643 | UCAGCGGG A UACAGUAC | 1394 | GTACTGTA GGCTAGCTACAACGA CCCGCTGA | 6191 |
| 5645 | AGCGGGAU A CAGUACCU | 1395 | AGGTACTG GGCTAGCTACAACGA ATCCCGCT | 6192 |
| 5648 | GGGAUACA G UACCUAGC | 1396 | GCTAGGTA GGCTAGCTACAACGA TGTATCCC | 6193 |
| 5650 | GAUACAGU A CCUAGCAG | 1397 | CTGCTAGG GGCTAGCTACAACGA ACTGTATC | 6194 |
| 5655 | AGUACCUA G CAGGCUUG | 1398 | CAAGCCTG GGCTAGCTACAACGA TAGGTACT | 6195 |
| 5659 | CCUAGCAG G CUUGUCCA | 1399 | TGGACAAG GGCTAGCTACAACGA CTGCTAGG | 6196 |
| 5663 | GCAGGCUU G UCCACUCU | 1400 | AGAGTGGA GGCTAGCTACAACGA AAGCCTGC | 6197 |
| 5667 | GCUUGUCC A CUCUGCCU | 1401 | AGGCAGAG GGCTAGCTACAACGA GGACAAGC | 6198 |
| 5672 | UCCACUCU G CCUGGGAA | 1402 | TTCCCAGG GGCTAGCTACAACGA AGAGTGGA | 6199 |
| 5680 | GCCUGGGA A CCCCGCGA | 1403 | TCGCGGGG GGCTAGCTACAACGA TCCCAGGC | 6200 |
| 5685 | GGAACCCC G CGAUAGCA | 1404 | TGCTATCG GGCTAGCTACAACGA GGGGTTCC | 6201 |
| 5688 | ACCCCGCG A UAGCAUCA | 1405 | TGATGCTA GGCTAGCTACAACGA CGCGGGGT | 6202 |
| 5691 | CCGCGAUA G CAUCAUUG | 1406 | CAATGATG GGCTAGCTACAACGA TATCGCGG | 6203 |
| 5693 | GCGAUAGC A UCAUUGAU | 1407 | ATCAATGA GGCTAGCTACAACGA GCTATCGC | 6204 |
| 5696 | AUAGCAUC A UUGAUGGC | 1408 | GCCATCAA GGCTAGCTACAACGA GATGCTAT | 6205 |
| 5700 | CAUCAUUG A UGGCAUUC | 1409 | GAATGCGA GGCTAGCTACAACGA CAATGATG | 6206 |
| 5703 | CAUUGAUG G CAUUCACA | 1410 | TGTGAATG GGCTAGCTACAACGA CATCAATG | 6207 |
| 5705 | UUGAUGGC A UUCACAGC | 1411 | GCTGTGAA GGCTAGCTACAACGA GCCATCAA | 6208 |
| 5709 | UGGCAUUC A CAGCCUCC | 1412 | GGAGGCTG GGCTAGCTACAACGA GAATGCCA | 6209 |
| 5712 | CAUUCACA G CCUCCAUC | 1413 | GATGGAGG GGCTAGCTACAACGA TGTGAATG | 6210 |
| 5718 | CAGCCUCC A UCACCAGC | 1414 | GCTGGTGA GGCTAGCTACAACGA GGAGGCTG | 6211 |
| 5721 | CCUCCAUC A CCAGCCCG | 1415 | CGGGCTGG GGCTAGCTACAACGA GATGGAGG | 6212 |
| 5725 | CAUCACGA G CCCGCUCA | 1416 | TGAGCGGG GGCTAGCTACAACGA TGGTGATG | 6213 |
| 5729 | ACCAGCCC G CUCACCAC | 1417 | GTGGTGAG GGCTAGCTACAACGA GGGCTGGT | 6214 |
| 5733 | GCCCGCUC A CCACCCAA | 1418 | TTGGGTGG GGCTAGCTACAACGA GAGCGGGC | 6215 |
| 5736 | CGCUCACC A CCCAAAGC | 1419 | GCTTTGGG GGCTAGCTACAACGA GGTGAGCG | 6216 |
| 5743 | CACCCAAA G CACCCUCC | 1420 | GGAGGGTG GGCTAGCTACAACGA TTTGGGTG | 6217 |
| 5745 | CCCAAAGC A CCCUCCUG | 1421 | CAGGAGGG GGCTAGCTACAACGA GCTTTGGG | 6218 |
| 5753 | ACCCUCCU G UUCAACAU | 1422 | ATGTTGAA GGCTAGCTACAACGA AGGAGGGT | 6219 |
| 5758 | CCUGUUCA A CAUCUUGG | 1423 | CCAAGATG GGCTAGCTACAACGA TGAACAGG | 6220 |
| 5760 | UGUUCAAC A UCUUGGGA | 1424 | TCCCAAGA GGCTAGCTACAACGA GTTGAACA | 6221 |
| 5771 | UUGGGAGG G UGGGUGGC | 1425 | GCCACCGA GGCTAGCTACAACGA CCTCCCAA | 6222 |
| 5775 | GACGGUGG G UGGCCGCC | 1426 | GCCGGCGA GGCTAGCTACAACGA CCACCCTC | 6223 |
| 5778 | GGUGGGUG G CCGCCCAA | 1427 | TTGGGCGG GGCTAGCTACAACGA CACCCACC | 6224 |
| 5781 | GGGUGGCC G CCCAACUC | 1428 | GAGTTGGG GGCTAGCTACAACGA GGCCACCC | 6225 |
| 5786 | GCCGCCCA A CUCGCUCC | 1429 | GGAGCGAG GGCTAGCTACAACGA TGGGCGGC | 6226 |
| 5790 | CCCAACUC G CUCCCCCC | 1430 | GGGGGGAG GGCTAGCTACAACGA GAGTTGGG | 6227 |
| 5802 | CCCCCAGA G CCGUUUCG | 1431 | CGAAACGG GGCTAGCTACAACGA TCTGGGGG | 6228 |
| 5805 | CCAGAGCC G UUUCGGCC | 1432 | GGCCGAAA GGCTAGCTACAACGA GGCTCTGG | 6229 |
| 5811 | CCGUUUCC G CCUUCGUG | 1433 | CACGAAGG GGCTAGCTACAACGA CGAAACGG | 6230 |
| 5817 | CGGCCUUC G UGGGCGCC | 1434 | GGCGCCGA GGCTAGCTACAACGA GAAGGCCG | 6231 |
| 5821 | CUUCGUGG G CGCCGGCA | 1435 | TGCCGGCG GGCTAGCTACAACGA CCACGAAG | 6232 |
| 5823 | UCGUGGGC G CCGGCAUC | 1436 | GATGCCGG GGCTAGCTACAACGA GCCCACGA | 6233 |
| 5827 | CGCCGCCG G CAUCGCUG | 1437 | CACCGATG GGCTAGCTACAACGA CGGCCCCC | 6234 |
| 5829 | CCGCCGGC A UCCCUGGC | 1438 | GCCAGCGA GGCTAGCTACAACGA GCCGGCGC | 6235 |
| 5832 | CCCCCAUC G CUGGCGCG | 1439 | CGCGCCAG GGCTAGCTACAACGA GATGCCGG | 6236 |
| 5836 | CAUCGCUG G CGCGCCUG | 1440 | CAGCCGCG GGCTAGCTACAACGA CAGCGATC | 6237 |
| 5838 | UCCCUGGC G CGCCUGUU | 1441 | AACACCCG GGCTAGCTACAACGA GCCAGCGA | 6238 |
| 5841 | CUGGCGCG G CUGUUCGC | 1442 | CCCAACAG GGCTAGCTACAACGA CGCGCCAG | 6239 |
| 5844 | CCGCGGCU G UUGGCAGC | 1443 | CCTCCCAA GGCTAGCTACAACGA AGCCGCGC | 6240 |
| 5848 | GGCUCUUG G CAGCAUAC | 1444 | CTATGCTC GGCTAGCTACAACGA CAACAGCC | 6241 |
| 5851 | UCUUGCGA G CAUAGGCC | 1445 | GGCCTATG GGCTAGCTACAACGA TCCCAACA | 6242 |
| 5853 | UUGGCACC A UAGCCCUU | 1446 | AAGGCCTA CCCTAGCTACAACGA CCTCCCAA | 6243 |
| 5857 | CAGCAUAC G CCUUGGGA | 1447 | TCCCAAGC CCCTAGCTACAACGA CTATCCTC | 6244 |
| 5868 | UUGCCAAG G UGCUUGUA | 1448 | TACAAGCA GGCTAGCTACAACGA CTTCCCAA | 6245 |
| 5870 | GGGAAGGU G CUUGUACA | 1449 | TCTACAAG GGCTAGCTACAACGA ACCTTCCC | 6246 |
| 5874 | ACGUCCUU G UACACAUU | 1450 | AATGTCTA GGCTAGCTACAACGA AACCACCT | 6247 |
| 5878 | CCUUGUAG A CAUUCUGG | 1451 | CCAGAATG GGCTAGCTACAACGA CTACAAGC | 6248 |
| 5880 | UUGUAGAC A UUCUGGCG | 1452 | CGCCAGAA GGCTAGCTACAACGA GTCTACAA | 6249 |
| 5886 | ACAUUCUG G CGGGCUAU | 1453 | ATAGCCCG GGCTAGCTACAACGA CAGAATGT | 6250 |
| 5890 | UCUGGCGG G CUAUGCAG | 1454 | CTCCATAC GGCTAGCTACAACGA CCGCCAGA | 6251 |
| 5893 | GGCGGGCU A UGGACCAG | 1455 | CTGCTCGA GGCTAGCTACAACGA AGCCCGCC | 6252 |
| 5898 | CCUAUCGA G CACGAGUG | 1456 | CACTCCTG GGCTAGCTACAACGA TCCATAGC | 6253 |
| 5904 | GAGCACGA G UGGCGGCU | 1457 | ACCCGCGA GGCTAGCTACAACGA TCCTGCTC | 6254 |
| 5907 | CAGGAGUG G CGGGUGCU | 1458 | AGCACCCG GGCTAGCTACAACGA CACTCCTG | 6255 |
| 5911 | AGUGCCGG G UGCUCUCG | 1459 | CCAGAGCA GGCTAGCTACAACGA CCGCCACT | 6256 |
| 5913 | UGGCGGGU G CUCUCGUG | 1460 | CACGAGAG GGCTAGCTACAACGA ACCCGCCA | 6257 |
| 5919 | CUGCUCUC G UGCCCUUC | 1461 | GAACGCGA GGCTAGCTACAACGA GAGAGCAC | 6258 |
| 5922 | CUCUCCUG G CCUUCAAC | 1462 | CTTGAAGC GGCTAGCTACAACGA CACCACAG | 6259 |
| 5931 | CCUUCAAG G UCAUCACC | 1463 | CCTCATCA GGCTAGCTACAACGA CTTCAAGC | 6260 |
| 5934 | UCAAGCUC A UCAGCGGG | 1464 | CCCCCTCA GGCTAGCTACAACGA GACCTTCA | 6261 |
| 5938 | CCUCAUCA G CCCGCACA | 1465 | TCTCCCCC GGCTAGCTACAACGA TCATGACC | 6262 |
| 5946 | CCCCCCAC A UCCCUUCU | 1466 | ACAACCGA GGCTAGCTACAACGA CTCCCCCC | 6263 |
| 5948 | CCGCACAU G CCUUCUAC | 1467 | GTACAAGC GGCTAGCTACAACGA ATCTCCCC | 6264 |
| 5955 | UCCCUUCU A CCGAGGAC | 1468 | CTCCTCCC GGCTAGCTACAACGA ACAACGCA | 6265 |
| 5962 | UACCGACG A CCUGGUCA | 1469 | TGACCACC GGCTAGCTACAACGA CCTCGGTA | 6266 |
| 5967 | AGGACCUG G UCAACUUA | 1470 | TAACTTGA GGCTAGCTACAACGA CAGGTCCT | 6267 |
| 5971 | CCUCCUCA A CUUACUCC | 1471 | GCACTAAC GGCTAGCTACAACGA TGACCAGC | 6268 |
| 5975 | GUCAACUU A CUCCCUGC | 1472 | GCACGCAC GGCTAGCTACAACGA AACTTCAC | 6269 |
| 5982 | UACUCCCU G CCAUCCUC | 1473 | CAGCATCC GGCTAGCTACAACGA ACCCAGTA | 6270 |
| 5985 | UCCCUGCC A UCCUCUCU | 1474 | ACACAGGA GGCTAGCTACAACGA GGCACCCA | 6271 |
| 5998 | CUCUCCUC G CCCCCUGG | 1475 | CCACCCCC GGCTAGCTACAACGA CACCACAC | 6272 |
| 6000 | CUCCUGGC G CCCUGGUC | 1476 | CACCACGC GGCTAGCTACAACGA CCCACGAG | 6273 |
| 6006 | GCGCCCUG G UCGUCGGG | 1477 | CCCGACGA GGCTAGCTACAACGA CAGGCCCC | 6274 |
| 6009 | CCCUGGUC G UCGGGGUG | 1478 | CACCCCGA GGCTAGCTACAACGA CACCAGGC | 6275 |
| 6015 | UCGUCGGG G UGGUGUGC | 1479 | GCACACGA GGCTAGCTACAACGA CCCGACGA | 6276 |
| 6018 | UCGGGGUG G UGUGCGCA | 1480 | TGCGCACA GGCTAGCTACAACGA CACCCCGA | 6277 |
| 6020 | GGGGUGGU G UGCGCAGC | 1481 | GCTGCGCA GGCTAGCTACAACGA ACCACCCC | 6278 |
| 6022 | GGUGGUGU G CGCAGCGA | 1482 | TCGCTGCG GGCTAGCTACAACGA ACACCACC | 6279 |
| 6024 | UGGUGUGC G CAGCGAUA | 1483 | TATCGCTG GGCTAGCTACAACGA GCACACCA | 6280 |
| 6027 | UGUGCGCA G CGAUACUG | 1484 | CAGTATCG GGCTAGCTACAACGA TGCGCACA | 6281 |
| 6030 | GCGCAGCG A UACUGCGU | 1485 | ACGCAGTA GGCTAGCTACAACGA CGCTGCGC | 6282 |
| 6032 | GCAGCGAU A CUGCGUCG | 1486 | CGACGCAG GGCTAGCTACAACGA ATCGCTGC | 6283 |
| 6035 | GCGAUACU G CGUCGGCA | 1487 | TGCCGACG GGCTAGCTACAACGA AGTATCGC | 6284 |
| 6037 | GAUACUGC G UCGGCAUG | 1488 | CATGCCGA GGCTAGCTACAACGA GCAGTATC | 6285 |
| 6041 | CUGCGUCG G CAUGUGGG | 1489 | CCCACATG GGCTAGCTACAACGA CCACCCAC | 6286 |
| 6043 | GCCUCGGC A UGUGGGCC | 1490 | GGCCCACA GGCTAGCTACAACGA GCCGACGC | 6287 |
| 6045 | CUCCCCAU G UGGGCCCA | 1491 | TGGGCCGA GGCTAGCTACAACGA ATGCCGAC | 6288 |
| 6049 | GCAUGUGG G CCCAGGAG | 1492 | CTCCTGGG GGCTAGCTACAACGA CCACATGC | 6289 |
| 6061 | AGCAGAGG G CGCUGUGC | 1493 | GCACAGCG GGCTAGCTACAACGA CCTCTCCT | 6290 |
| 6063 | GACACCCC G CUGUCCAG | 1494 | CTGCACAG GGCTAGCTACAACGA GCCCTCTC | 6291 |
| 6066 | AGGGCGCU G UGCAGUGG | 1495 | CCACTGCA GGCTAGCTACAACGA AGCGCCCT | 6292 |
| 6068 | CGCGCUGU G CAGUGGAU | 1496 | ATCCACTG GGCTAGCTACAACGA ACAGCGCC | 6293 |
| 6071 | GCUCUCGA G UGGAUGAA | 1497 | TTCATCGA GGCTAGCTACAACGA TCCACACC | 6294 |
| 6075 | UCCACUGG A UCAAUCGC | 1498 | CCCATTCA GGCTAGCTACAACGA CCACTGCA | 6295 |
| 6079 | GUGGAUCA A UCGCCUCA | 1499 | TCAGCCGA GGCTAGCTACAACGA TCATCCAC | 6296 |
| 6083 | AUGAAUCG G CUGAUAGC | 1500 | GCTATCAG GGCTAGCTACAACGA CGATTCAT | 6297 |
| 6087 | AUCGGCUG A UAGCGUUC | 1501 | CAACGCTA GGCTAGCTACAACGA CACCCCAT | 6298 |
| 6090 | CCCUGAUA G CGUUCGCU | 1502 | AGCGAACG GGCTAGCTACAACGA TATCAGCC | 6299 |
| 6092 | CUCAUAGC G UUCGCUUC | 1503 | GAAGCGAA GGCTAGCTACAACGA GCTATCAG | 6300 |
| 6096 | UAGCGUUC G CUUCGCGG | 1504 | CCGCGAAG GGCTAGCTACAACGA GAACGCTA | 6301 |
| 6101 | UUCGCUUC G CGGGCCAA | 1505 | TTGCCCCG GGCTAGCTACAACGA GAAGCGAA | 6302 |
| 6106 | UUCGCGGG G CAACCAUG | 1506 | CATGGTTG GGCTAGCTACAACGA CCCGCGAA | 6303 |
| 6109 | GCCCGGCA A CCAUGUCU | 1507 | AGACATGG GGCTAGCTACAACGA TGCCCCGC | 6304 |
| 6112 | GGGCAACC A UGUCUCCC | 1508 | GGGAGACA GGCTAGCTACAACGA GGTTGCCC | 6305 |
| 6114 | CCAACCAU G UCUCCCCC | 1509 | GGGGGACA GGCTAGCTACAACGA ATGGTTGC | 6306 |
| 6123 | UCUCCCCC A CGCACUAU | 1510 | ATAGTGCG GGCTAGCTACAACGA G3GGGAGA | 6307 |
| 6125 | UCCCCCAC G CACUAUGU | 1511 | ACATAGTC GGCTAGCTACAACGA CTGGGGGA | 6308 |
| 6127 | CCCCACCC A CUAUCUGC | 1512 | CCACATAG GGCTAGCTACAACGA GCCTGGGG | 6309 |
| 6130 | CACGCACU A UGUCCCUG | 1513 | CAGGCACA GGCTAGCTACAACGA AGTGCGTG | 6310 |
| 6132 | CCCACUAU G UGCCUCAG | 1514 | CTCACCGA GGCTAGCTACAACGA ATAGTCCC | 6311 |
| 6134 | CACUAUCU G CCUGAGAG | 1515 | CTCTCAGC GGCTAGCTACAACGA ACATAGTG | 6312 |
| 6142 | GCCUGACA G CGACCCAC | 1516 | CTGCGTCG GGCTAGCTACAACGA TCTCACCC | 6313 |
| 6145 | UCACAGCG A CGCAGCGC | 1517 | CCGCTCCC GGCTAGCTACAACGA CGCTCTCA | 6314 |
| 6147 | AGAGCGAC G CAGCGGCG | 1518 | CGCCGCTG GGCTAGCTACAACGA GTCGCTCT | 6315 |
| 6150 | GCCACCGA G CGCCGCCC | 1519 | GCGCGCCG GGCTAGCTACAACGA TGCGTCCC | 6316 |
| 6153 | ACGCAGCG G CCCGCGUC | 1520 | CACCCCCG GGCTAGCTACAACGA CCCTCCGT | 6317 |
| 6155 | CCACCCGC G CGCCUCAC | 1521 | CTCACCCC GGCTAGCTACAACGA GCCCCTCC | 6318 |
| 6157 | ACCGGCCC G CGUCACAC | 1522 | GTGTCACG GGCTAGCTACAACGA GCCCCGCT | 6319 |
| 6159 | CCCCGCCC G UCACACAA | 1523 | TTCTCTCA GGCTAGCTACAACGA GCGCGCCG | 6320 |
| 6162 | CGCCCGUC A CACAAAUC | 1524 | CATTTGTG GGCTAGCTACAACGA GACGCGCG | 6321 |
| 6164 | CCCCUCAC A CAAAUCCU | 1525 | ACCATTTG GGCTAGCTACAACGA GTCACCCG | 6322 |
| 6168 | UCACACAA A UCCUCUCC | 1526 | CCAGAGGA GGCTAGCTACAACGA TTGTGTGA | 6323 |
| 6178 | CCUCUCGA G CCUCACGA | 1527 | TCCTGAGG GGCTAGCTACAACGA TCCACAGC | 6324 |
| 6283 | CCACCCUC A CCAUCACU | 1528 | AGTCATGC GGCTAGCTACAACGA GACCCTGG | 6325 |
| 6186 | CCCUCACC A UCACUCAG | 1529 | CTCACTGA GGCTAGCTACAACGA CCTCACCC | 6326 |
| 6189 | UCACCAUC A CUCACCUG | 1530 | CAGCTGAC GGCTAGCTACAACGA GATGGTGA | 6327 |
| 6194 | AUCACUCA G CUCCUGAG | 1531 | CTCAGCAG GGCTAGCTACAACGA TGACTCAT | 6328 |
| 6197 | ACUCAGCU G CUGAGGAG | 1532 | CTCCTCAG GGCTAGCTACAACGA ACCTCAGT | 6329 |
| 6206 | CUGAGGAG G CUCCAUCA | 1533 | TCATGCAC GGCTAGCTACAACGA CTCCTCAG | 6330 |
| 6211 | GAGGCUCC A UCACUGGA | 1534 | TCCACTCA GGCTAGCTACAACGA CGACCCTC | 6331 |
| 6215 | CUCCAUCA G UGGAUCAA | 1535 | TTGATCGA GGCTAGCTACAACGA TCATCGAC | 6332 |
| 6219 | AUCAGUGG A UCAAUGAG | 1536 | CTCATTGA GGCTAGCTACAACGA CCACTGAT | 6333 |
| 6223 | GUGGAUCA A UGAGGACU | 1537 | AGTCCTCA GGCTAGCTACAACGA TGATCCAC | 6334 |
| 6229 | CAAUGAGG A CUGCUCGA | 1538 | TGGAGCAG GGCTAGCTACAACGA CCTCATTG | 6335 |
| 6232 | UGAGGACU G CUCCACGC | 1539 | GCGTGGAG GGCTAGCTACAACGA AGTCCTCA | 6336 |
| 6237 | ACUGCUCC A CGCCAUGU | 1540 | ACATGGCG GGCTAGCTACAACGA GGAGCAGT | 6337 |
| 6239 | UGCUCCAC G CCAUGUUC | 1541 | GAACATGG GGCTAGCTACAACGA GTGGAGCA | 6338 |
| 6242 | UCCACGCC A UGUUCCCG | 1542 | CCGGAACA GGCTAGCTACAACGA GCCGTGGA | 6339 |
| 6244 | CACGCCAU G UUCCGGCU | 1543 | AGCCGGAA GGCTAGCTACAACGA ATGGCGTC | 6340 |
| 6250 | AUGUUCCG G CUCGUGGC | 1544 | GCCACGAG GGCTAGCTACAACGA CGGAACAT | 6341 |
| 6254 | UCCGGCUC G UGGCUAAG | 1545 | CTTAGCGA GGCTAGCTACAACGA GAGCCGGA | 6342 |
| 6257 | GGCUCGUG G CUAAGGGA | 1546 | TCCCTTAG GGCTAGCTACAACGA CACGAGCC | 6343 |
| 6265 | GCUAAGGG A UGUUUGGG | 1547 | CCCAAACA GGCTAGCTACAACGA CCCTTAGC | 6344 |
| 6267 | UAAGGGAU G UUUGGGAC | 1548 | GTCCCAAA GGCTAGCTACAACGA ATCCCTTA | 6345 |
| 6274 | UGUUUGGG A CUGGAUAU | 1549 | ATATCCAG GGCTAGCTACAACGA CCCAAACA | 6346 |
| 6279 | GGGACUGG A UAUGCACG | 1550 | CGTGCATA GGCTAGCTACAACGA CCAGTCCC | 6347 |
| 6281 | GACUGGAU A UGCACGGU | 1551 | ACCGTGCA GGCTAGCTACAACGA ATCCAGTC | 6348 |
| 6283 | CUGGAUAU G CACGGUGU | 1552 | ACACCGTG GGCTAGCTACAACGA ATATCCAG | 6349 |
| 6285 | GGAUAUGC A CGCUGUUG | 1553 | CAACACCG GGCTAGCTACAACGA GCATATCC | 6350 |
| 6288 | UAUGCACG G UGUUGACU | 1554 | AGTCAACA GGCTAGCTACAACGA CGTGCATA | 6351 |
| 6290 | UGCACGGU G UUGACUGA | 1555 | TCAGTCAA GGCTAGCTACAACGA ACCGTGCA | 6352 |
| 6294 | CGGUGUUG A CUGACUUC | 1556 | GAAGTCAG GGCTAGCTACAACGA CAACACCG | 6353 |
| 6298 | GUUGACUG A CUUCAAGA | 1557 | TCTTGAAG GGCTAGCTACAACGA CAGTCAAC | 6354 |
| 6306 | ACUUCAAG A CCUGGCUU | 1558 | AAGCCAGG GGCTAGCTACAACGA CTTGAAGT | 6355 |
| 6311 | AAGACCUG G CUUCAGUC | 1559 | CACTGAAG GGCTAGCTACAACGA CAGGTCTT | 6356 |
| 6317 | UGGCUUCA G UCCAAGCU | 1560 | AGCTTGGA GGCTAGCTACAACGA TGAAGCCA | 6357 |
| 6323 | CAGUCCAA G CUCCUGCC | 1561 | CGCAGGAG GGCTAGCTACAACGA TTGGACTG | 6358 |
| 6329 | AAGCUCCU G CCGCGGUU | 1562 | AACCGCGG GGCTAGCTACAACGA AGGAGCTT | 6359 |
| 6332 | CUCCUGCC G CGGUUGCC | 1563 | CGCAACCG GGCTAGCTACAACGA GGCAGGAG | 6360 |
| 6335 | CUGCCCCC G UUGCCGGG | 1564 | CCCGGCAA GGCTAGCTACAACGA CGCGGCAG | 6361 |
| 6338 | CCGCGGUU G CCGGGAGU | 1565 | ACTCCCGG GGCTAGCTACAACGA AACCGCGG | 6362 |
| 6345 | UGCCGGGA G UCCCUUUC | 1566 | GAAAGGGA GGCTAGCTACAACGA TCCCCCCA | 6363 |
| 6359 | UUCUUCUC A UGCCAACG | 1567 | CGTTGGCA GGCTAGCTACAACGA GAGAAGAA | 6364 |
| 6361 | CUUCUCAU G CCAACGUG | 1568 | CACGTTGG GGCTAGCTACAACGA ATGAGAAG | 6365 |
| 6365 | UCAUGCCA A CGUGGGUA | 1569 | TACCCACG GGCTAGCTACAACGA TGGCATGA | 6366 |
| 6367 | AUGCCAAC G UGGGUACA | 1570 | TGTACCGA GGCTAGCTACAACGA GTTGGCAT | 6367 |
| 6371 | CAACGUGG G UACAGGGG | 1571 | CCCCTGTA GGCTAGCTACAACGA CCACGTTG | 6368 |
| 6373 | ACCUGGGU A CAGGGGGG | 1572 | CCCCCCTG GGCTAGCTACAACGA ACCCACGT | 6369 |
| 6381 | ACAGGGGG G UCUGGCGG | 1573 | CCGCCACA GGCTAGCTACAACGA CCCCCTGT | 6370 |
| 6386 | GGGCUCUC G CGGGGACA | 1574 | TCTCCCCG GGCTAGCTACAACGA CAGACCCC | 6371 |
| 6394 | GCGGGGAC A CGCUAUCA | 1575 | TGATACCG GGCTAGCTACAACGA CTCCCCGC | 6372 |
| 6397 | GCGAGACC G UAUCAUCC | 1576 | CCATCATA GGCTAGCTACAACGA CGTCTCCC | 6373 |
| 6399 | CAGACCCU A UCAUGCAA | 1577 | TTCCATCA GGCTAGCTACAACGA ACCGTCTC | 6374 |
| 6402 | ACGGUAUC A UGCAAACC | 1578 | CGTTTCGA GGCTAGCTACAACGA GATACCGT | 6375 |
| 6404 | GCUAUCAU G CAAACCAC | 1579 | GTGGTTTG GGCTAGCTACAACGA ATGATACC | 6376 |
| 6408 | UCAUCCAA A CCACCUCC | 1580 | GCACGTGG GGCTAGCTACAACGA TTGCATGA | 6377 |
| 6411 | UCCAAACC A CCUGCCGA | 1581 | TGGCCACG GGCTAGCTACAACGA CGTTTGCA | 6378 |
| 6415 | AACCACCU G CCCAUGCG | 1582 | CGCATCGC GGCTAGCTACAACGA ACCTGGTT | 6379 |
| 6419 | ACCUGCCC A UGCCGAGC | 1583 | CCTCCCGA GGCTAGCTACAACGA GGCCAGCT | 6380 |
| 6421 | CUCCCCAU G CGGACCCC | 1584 | GCGCTCCG GGCTAGCTACAACGA ATGGCCAC | 6381 |
| 6426 | CAUGCGGA G CGCAGAUC | 1585 | GATCTGCG GGCTAGCTACAACGA TCCGCATC | 6382 |
| 6428 | UCCCCAGC G CACAUCAC | 1586 | GTCATCTC GGCTAGCTACAACGA GCTCCGCA | 6383 |
| 6432 | GAGCGCAG A UCACUGGA | 1587 | TCCAGTCA GGCTAGCTACAACGA CTGCGCTC | 6384 |
| 6435 | CCCACAUC A CUGGACAU | 1588 | ATGTCCAC GGCTAGCTACAACGA CATCTGCC | 6385 |
| 6440 | AUCACUGG A CAUGUCAA | 1589 | TTGACATC GGCTAGCTACAACGA CCACTGAT | 6386 |
| 6442 | CACUGGAC A UGUCAAGA | 1590 | TCTTGACA GGCTAGCTACAACGA GTCCAGTC | 6387 |
| 6444 | CUGGACAU G UCAAGAAC | 1591 | GTTCTTGA GGCTAGCTACAACGA ATGTCCAC | 6388 |
| 6451 | UGUCAAGA A CGGUUCCA | 1592 | TGGAACCG GGCTAGCTACAACGA TCTTGACA | 6389 |
| 6454 | CAAGAACG G UUCCAUGA | 1593 | TCATGGAA GGCTAGCTACAACGA CGTTCTTG | 6390 |
| 6459 | ACGGUUCC A UGAGGAUC | 1594 | GATCCTCA GGCTAGCTACAACGA GGAACCGT | 6391 |
| 6465 | CCAUGAGG A UCGUCGGG | 1595 | CCCGACGA GGCTAGCTACAACGA CCTCATGG | 6392 |
| 6468 | UGAGGAUC G UCGGGCCU | 1596 | AGGCCCGA GGCTAGCTACAACGA GATCCTCA | 6393 |
| 6473 | AUCGUCGG G CCUAAGAC | 1597 | GTCTTAGG GGCTAGCTACAACGA CCGACGAT | 6394 |
| 6480 | GGCCUAAG A CCUGUAGC | 1598 | GCTACAGG GGCTAGCTACAACGA CTTAGGCC | 6395 |
| 6484 | UAAGACCU G UAGCAACA | 1599 | TGTTGCTA GGCTAGCTACAACGA AGGTCTTA | 6396 |
| 6487 | GACCUGUA G CAACACGU | 1600 | ACGTGTTG GGCTAGCTACAACGA TACAGGTC | 6397 |
| 6490 | CUGUAGCA A CACGUGGC | 1601 | GCCACGTG GGCTAGCTACAACGA TGCTACAG | 6398 |
| 6492 | GUAGCAAC A CGUGGCAU | 1602 | ATGCCACG GGCTAGCTACAACGA GTTGCTAC | 6399 |
| 6494 | AGCAACAC G UGGCAUGG | 1603 | CCATGCGA GGCTAGCTACAACGA GTGTTGCT | 6400 |
| 6497 | AACACGUG G CAUCGAAC | 1604 | GTTCCATG GGCTAGCTACAACGA CACGTGTT | 6401 |
| 6499 | CACGUGGC A UGGAACAU | 1605 | ATGTTCGA GGCTAGCTACAACGA GCCACGTG | 6402 |
| 6504 | GGCAUGGA A CAUUCCCC | 1606 | GGGGAATG GGCTAGCTACAACGA TCCATGCC | 6403 |
| 6506 | CAUGGAAC A UUCCCCAU | 1607 | ATGGGGAA GGCTAGCTACAACGA GTTCCATG | 6404 |
| 6513 | CAUUCCCC A UCAACGCA | 1608 | TGCGTTGA GGCTAGCTACAACGA GGGGAATG | 6405 |
| 6517 | CCCCAUCA A CGCAUACA | 1609 | TGTATGCG GGCTAGCTACAACGA TGATGGGG | 6406 |
| 6519 | CCAUCAAC G CAUACACC | 1610 | GGTGTATG GGCTAGCTACAACGA GTTGATGG | 6407 |
| 6521 | AUCAACGC A UACACCAC | 1611 | GTGGTGTA GGCTAGCTACAACGA GCGTTGAT | 6408 |
| 6523 | CAACGCAU A CACCACGG | 1612 | CCGTGGTG GGCTAGCTACAACGA ATGCGTTG | 6409 |
| 6525 | ACGCAUAC A CCACGGGC | 1613 | GCCCGTGG GGCTAGCTACAACGA GTATGCGT | 6410 |
| 6528 | CAUACACC A CGGGCCCC | 1614 | GGGGCCCG GGCTAGCTACAACGA GGTGTATG | 6411 |
| 6532 | CACCACGG G CCCCUGCA | 1615 | TGCAGGGG GGCTAGCTACAACGA CCGTGGTG | 6412 |
| 6538 | GGGCCCCU G CACACCCU | 1616 | AGGGTGTG GGCTAGCTACAACGA AGGGGCCC | 6413 |
| 6540 | GCCCCUGC A CACCCUCC | 1617 | GGAGGGTG GGCTAGCTACAACGA GCAGGGGC | 6414 |
| 6542 | CCCUGCAC A CCCUCCCC | 1618 | GGGGAGGG GGCTAGCTACAACGA GTGCAGGG | 6415 |
| 6552 | CCUCCCCG G CGCCAAAC | 1619 | GTTTGGCG GGCTAGCTACAACGA CGGGGAGG | 6416 |
| 6554 | UCCCCGGC G CCAAACUA | 1620 | TAGTTTGG GGCTAGCTACAACGA GCCGGGGA | 6417 |
| 6559 | GGCGCCAA A CUAUUCUA | 1621 | TAGAATAG GGCTAGCTACAACGA TTGGCGCC | 6418 |
| 6562 | GCCAAACU A UUCUAGGG | 1622 | GGCTAGAA GGCTAGCTACAACGA AGTTTGGC | 6419 |
| 6570 | AUUCUAGG G CGCUAUGG | 1623 | CCATAGCG GGCTAGCTACAACGA CCTAGAAT | 6420 |
| 6572 | UCUAGGGC G CUAUGGCG | 1624 | CGCCATAG GGCTAGCTACAACGA GGGCTAGA | 6421 |
| 6575 | AGGGCGCU A UGGCGGGU | 1625 | ACCCGCGA GGCTAGCTACAACGA AGCGCCCT | 6422 |
| 6578 | GCGCUAUG G CGGGUGGC | 1626 | GCCACCCG GGCTAGCTACAACGA CATAGCGC | 6423 |
| 6582 | UAUGGCGG G UGGCCGCU | 1627 | AGCGGCGA GGCTAGCTACAACGA CCGCCATA | 6424 |
| 6585 | GGCGGCUG G CCGCUGAG | 1628 | CTCAGCGG GGCTAGCTACAACGA CACCCGCC | 6425 |
| 6588 | GGGUGGCC G CUGAGGAG | 1629 | CTCCTCAG GGCTAGCTACAACGA GGCCACCC | 6426 |
| 6596 | GCUCACGA G UACGUGGA | 1630 | TCCACGTA GGCTAGCTACAACGA TCCTCAGC | 6427 |
| 6598 | UGACGAGU A CGUCCAGG | 1631 | CCTCCACG GGCTAGCTACAACGA ACTCCTCA | 6428 |
| 6600 | AGGAGUAC G UGGAGCUU | 1632 | AACCTCGA GGCTAGCTACAACGA GTACTCCT | 6429 |
| 6606 | ACGUGGAG G UUACGCGG | 1633 | CCGCGTAA GGCTAGCTACAACGA CTCCACCT | 6430 |
| 6609 | UGCAGGUU A CGCGGGUG | 1634 | CACCCCCG GGCTAGCTACAACGA AACCTCCA | 6431 |
| 6611 | GACGUUAC G CCCGUGGC | 1635 | CCCACCCC GGCTAGCTACAACGA GTAACCTC | 6432 |
| 6615 | UUACCCGG G UGCGGGAU | 1636 | ATCCCCGA GGCTAGCTACAACGA CCGCGTAA | 6433 |
| 6622 | GCUCGGGC A UUUCCACU | 1637 | AGTGGAAA GGCTAGCTACAACGA CCCCCACC | 6434 |
| 6628 | GGAUUUCC A CUACCUGA | 1638 | TCACGTAG GGCTAGCTACAACGA CGAAATCC | 6435 |
| 6631 | UUUCCACU A CGUCACGG | 1639 | CCGTCACG GGCTAGCTACAACGA AGTGGAAA | 6436 |
| 6633 | UCCACUAC G UGACCCGC | 1640 | GCCCGTCA GGCTAGCTACAACGA GTACTGGA | 6437 |
| 6636 | ACUACGUG A CGGGCAUG | 1641 | CATCCCCG GGCTAGCTACAACGA CACGTAGT | 6438 |
| 6640 | CGUCACCC G CAUGACGA | 1642 | TGGTCATG GGCTAGCTACAACGA CCGTCACG | 6439 |
| 6642 | UGACGGGC A UGACCACU | 1643 | AGTGGTCA GGCTAGCTACAACGA GCCCGTCA | 6440 |
| 6645 | CCCGCAUC A CCACUCAC | 1644 | CTCACTCG GGCTAGCTACAACGA CATGCCCG | 6441 |
| 6648 | GCAUGACC A CUGACAAC | 1645 | GTTGTCAG GGCTAGCTACAACGA GGTCATCC | 6442 |
| 6652 | GACCACUG A CAACGUAA | 1646 | TTACGTTC GGCTAGCTACAACGA CAGTGGTC | 6443 |
| 6655 | CACUGACA A CGUAAAAU | 1647 | ATTTTACG GGCTAGCTACAACGA TGTCAGTG | 6444 |
| 6657 | CUGACAAC G UAAAAUGC | 1648 | GCATTTTA GGCTAGCTACAACGA GTTGTCAG | 6445 |
| 6662 | AACGUAAA A UGCCCGUG | 1649 | CACGCGCA GGCTAGCTACAACGA TTTACGTT | 6446 |
| 6664 | CGUAAAAU G CCCGUGCC | 1650 | GGCACGGG GGCTAGCTACAACGA ATTTTACG | 6447 |
| 6668 | AAAUGCCC G UGCCACGU | 1651 | ACCTGGCA GGCTAGCTACAACGA GGGCATTT | 6448 |
| 6670 | AUGCCCGU G CCAGGUUC | 1652 | GAACCTGG GGCTAGCTACAACGA ACGGGCAT | 6449 |
| 6675 | CGUGCCAG G UUCCGCCC | 1653 | GGGCGGAA GGCTAGCTACAACGA CTGGCACG | 6450 |
| 6680 | CAGGUUCC G CCCCCCGA | 1654 | TCGGGGGG GGCTAGCTACAACGA GGAACCTG | 6451 |
| 6689 | CCCCCCGA A UUCUUCAC | 1655 | GTGAAGAA GGCTAGCTACAACGA TCGGGGGG | 6452 |
| 6696 | AAUUCUUC A CGGAAGUG | 1656 | CACTTCCG GGCTAGCTACAACGA GAAGAATT | 6453 |
| 6702 | UCACGGAA G UGGAUGGG | 1657 | CCCATCGA GGCTAGCTACAACGA TTCCGTGA | 6454 |
| 6706 | GGAAGUGG A UGGGGUAC | 1658 | GTACCCGA GGCTAGCTACAACGA CCACTTCC | 6455 |
| 6711 | UGGAUGGG G UACGCCUG | 1659 | CAGGCGTA GGCTAGCTACAACGA CCCATCCA | 6456 |
| 6713 | GAUGGGGU A CGCCUGCA | 1660 | TGCAGGCG GGCTAGCTACAACGA ACCCCATC | 6457 |
| 6715 | UGGGGUAC G CCUGCACA | 1661 | TGTGCAGG GGCTAGCTACAACGA GTACCCCA | 6458 |
| 6719 | GUACGCCU G CACAGAAA | 1662 | TTTCTGTG GGCTAGCTACAACGA AGGCGTAC | 6459 |
| 6721 | ACGCCUGC A CAGAAACG | 1663 | CGTTTCTG GGCTAGCTACAACGA GCAGGCGT | 6460 |
| 6727 | GCACAGAA A CGCUCCGG | 1664 | CCGGAGCG GGCTAGCTACAACGA TTCTGTGC | 6461 |
| 6729 | ACAGAAAC G CUCCGGCG | 1665 | CGCCGGAG GGCTAGCTACAACGA GTTTCTGT | 6462 |
| 6735 | ACGCUCCG G CGUGUGGA | 1666 | TCCACACG GGCTAGCTACAACGA CGGAGCGT | 6463 |
| 6737 | GCUCCGGC G UGUGGACC | 1667 | GGTCCACA GGCTAGCTACAACGA GCCGGAGC | 6464 |
| 6739 | UCCGGCGU G UCGACCUC | 1668 | GAGGTCGA GGCTAGCTACAACGA ACGCCGGA | 6465 |
| 6743 | GCGUGUGG A CCUCUCCU | 1669 | AGGAGAGG GGCTAGCTACAACGA CCACACGC | 6466 |
| 6752 | CCUCUCCU A CGGGAGGA | 1670 | TCCTCCCG GGCTAGCTACAACGA AGGAGAGG | 6467 |
| 6762 | GGGAGGAG G UCACAUUC | 1671 | GAATGTGA GGCTAGCTACAACGA CTCCTCCC | 6468 |
| 6765 | AGGAGGUC A CAUUCCAG | 1672 | CTGGAATG GGCTAGCTACAACGA GACCTCCT | 6469 |
| 6767 | GAGGUCAC A UUCCAGGU | 1673 | ACCTGGAA GGCTAGCTACAACGA GTGACCTC | 6470 |
| 6774 | CAUUCCAG G UCGGGCUC | 1674 | GAGCCCGA GGCTAGCTACAACGA CTGGAATG | 6471 |
| 6779 | CAGGUCGG G CUCAACCA | 1675 | TGGTTGAG GGCTAGCTACAACGA CCGACCTG | 6472 |
| 6784 | CGGGCUCA A CCAAUACC | 1676 | GGTATTGG GGCTAGCTACAACGA TGAGCCCG | 6473 |
| 6788 | CUCAACCA A UACCUGGU | 1677 | ACCAGGTA GGCTAGCTACAACGA TGGTTGAG | 6474 |
| 6790 | CAACCAAU A CCUGGUUG | 1678 | CAACCAGG GGCTAGCTACAACGA ATTGGTTG | 6475 |
| 6795 | AAUACCUG G UUG3GUCA | 1679 | TGACCCAA GGCTAGCTACAACGA CAGGTATT | 6476 |
| 6800 | CUGGUUGG G UCACAGCU | 1680 | AGCTGTGA GGCTAGCTACAACGA CCAACCAG | 6477 |
| 6803 | GUUGGGUC A CAGCUCCC | 1681 | GGGAGCTG GGCTAGCTACAACGA GACCCAAC | 6478 |
| 6806 | GUGUCACA G CUCCCAUG | 1682 | CATGGGAG GGCTAGCTACAACGA TGTGACCC | 6479 |
| 6812 | CAGCUCCC A UGCGAGCC | 1683 | GGCTCGCA GGCTAGCTACAACGA GGGAGCTG | 6480 |
| 6814 | GCUCCCAU G CGAGCCCG | 1684 | CGGGCTCG GGCTAGCTACAACGA ATGGGAGC | 6481 |
| 6818 | CCAUGCGA G CCCGAACC | 1685 | GGTTCGGG GGCTAGCTACAACGA TCGCATGG | 6482 |
| 6824 | GAGCCCCA A CCGGAUGU | 1686 | ACATCCGG GGCTAGCTACAACGA TCGGGCTC | 6483 |
| 6829 | CGAACCGG A UGUAGCAG | 1687 | CTGCTACA GGCTAGCTACAACGA CCGGTTCG | 6484 |
| 6831 | AACCGGAU G UAGCAGUG | 1688 | CACTGCTA GGCTAGCTACAACGA ATCCGGTT | 6485 |
| 6834 | CGGAUGUA G CAGUGCUC | 1689 | GAGCACTG GGCTAGCTACAACGA TACATCCG | 6486 |
| 6837 | AUGUAGCA G UGCUCACG | 1690 | CGTGAGCA GGCTAGCTACAACGA TGCTACAT | 6487 |
| 6839 | GUAGCAGU G CUCACGUC | 1691 | GACGTGAG GGCTAGCTACAACGA ACTGCTAC | 6488 |
| 6843 | CAGUGCUC A CGUCCAUG | 1692 | CATGGACG GGCTAGCTACAACGA GAGCACTG | 6489 |
| 6845 | GUGCUCAC G UCCAUGCU | 1693 | AGCATGGA GGCTAGCTACAACGA GTGAGCAC | 6490 |
| 6849 | UCACGUCC A UGCUCACC | 1694 | GGTGAGCA GGCTAGCTACAACGA GGACGTGA | 6491 |
| 6851 | ACGUCCAU G CUCACCGA | 1695 | TCGGTGAG GGCTAGCTACAACGA ATGGACGT | 6492 |
| 6855 | CCAUGCUC A CCGACCCC | 1696 | GGGGTCGG GGCTAGCTACAACGA GAGCATGG | 6493 |
| 6859 | GCUCACCG A CCCCUCCC | 1697 | GGGAGGGG GGCTAGCTACAACGA CGGTGAGC | 6494 |
| 6868 | CCCCUCCC A CAUCACAG | 1698 | CTGTAATG GGCTAGCTACAACGA GGGAGGGG | 6495 |
| 6870 | CCUCCCAC A UUACAGGA | 1699 | TCCTGTAA GGCTAGCTACAACGA GTGGGAGG | 6496 |
| 6873 | CCCACAUU A CAGGAGAG | 1700 | CTCTCCTG GGCTAGCTACAACGA AATGTGGG | 6497 |
| 6882 | CAGGAGAG A CGGCUAAG | 1701 | CTTAGCCG GGCTAGCTACAACGA CTCTCCTG | 6498 |
| 6885 | GAGAGACG G CUAAGCGU | 1702 | ACGCTTAG GGCTAGCTACAACGA CGTCTCTC | 6499 |
| 6890 | ACGGCUAA G CGUAGGCU | 1703 | AGCCTACG GGCTAGCTACAACGA TTAGCCGT | 6500 |
| 6892 | GGCUAAGC G UAGGCUGG | 1704 | CCAGCCTA GGCTAGCTACAACGA GCTTAGCC | 6501 |
| 6896 | AAGCGUAG G CUGGCCAG | 1705 | CTGGCCAG GGCTAGCTACAACGA CTACGCTT | 6502 |
| 6900 | GUAGGCUG G CCAGGGGG | 1706 | CCCCCTGG GGCTAGCTACAACGA CAGCCTAC | 6503 |
| 6908 | GCCAGGGG G UCUCCCCC | 1707 | GGGGGAGA GGCTAGCTACAACGA CCCCTGGC | 6504 |
| 6924 | CCUCCUUG G CCAGCUCC | 1708 | GGAGCTGG GGCTAGCTACAACGA CAAGGAGG | 6505 |
| 6928 | CUUGGCGA G CUCCUCAG | 1709 | CTGAGGAG GGCTAGCTACAACGA TGGCCAAG | 6506 |
| 6936 | GCUCCUCA G CUAGCCAG | 1710 | CTGGCTAG GGCTAGCTACAACGA TGAGGAGC | 6507 |
| 6940 | CUCAGCUA G CCAGCUGU | 1711 | ACAGCTGG GGCTAGCTACAACGA TAGCTGAG | 6508 |
| 6944 | GCUAGCGA G CUGUCUGC | 1712 | GCAGACAG GGCTAGCTACAACGA TGGCTAGC | 6509 |
| 6947 | AGCCAGCU G UCUGCGCC | 1713 | GGCGCAGA GGCTAGCTACAACGA AGCTGGCT | 6510 |
| 6951 | AGCUGUCU G CGCCUUCU | 1714 | AGAAGGCG GGCTAGCTACAACGA AGACAGCT | 6511 |
| 6953 | CUGUCUGC G CCUUCUUC | 1715 | GAAGAAGG GGCTAGCTACAACGA GCAGACAG | 6512 |
| 6966 | CUUCGAAG G CGACAUAC | 1716 | GTATGTCG GGCTAGCTACAACGA CTTCGAAG | 6513 |
| 6969 | CGAAGGCG A CAUACAUU | 1717 | AATGTATG GGCTAGCTACAACGA CGCCTTCG | 6514 |
| 6971 | AAGGCGAC A UACAUUAC | 1718 | GTAATGTA GGCTAGCTACAACGA GTCGCCTT | 6515 |
| 6973 | GGCGACAU A CAUUACCC | 1719 | GGGTAATG GGCTAGCTACAACGA ATGTCGCC | 6516 |
| 6975 | CGACAUAC A UUACCCAA | 1720 | TTGGGTAA GGCTAGCTACAACGA GTATGTCG | 6517 |
| 6978 | CAUACAUU A CCCAAUAU | 1721 | ATATTGGG GGCTAGCTACAACGA AATGTATG | 6518 |
| 6983 | AUUACCCA A UAUGACUC | 1722 | GAGTCATA GGCTAGCTACAACGA TGGGTAAT | 6519 |
| 6985 | UACCCAAU A UGACUCCC | 1723 | GGGAGTCA GGCTAGCTACAACGA ATTGGGTA | 6520 |
| 6988 | CCAAUAUG A CUCCCCAG | 1724 | CTGGGGAG GGCTAGCTACAACGA CATATTGG | 6521 |
| 6997 | CUCCCCAG A CUUUGACC | 1725 | GGTCAAAG GGCTAGCTACAACGA CTGGGGAG | 6522 |
| 7003 | AGACUUUG A CCUCAUCG | 1726 | CGATGAGG GGCTAGCTACAACGA CAAAGTCT | 6523 |
| 7008 | UUGACCUC A UCGAGGCC | 1727 | GGCCTCGA GGCTAGCTACAACGA GAGGTCAA | 6524 |
| 7014 | UCAUCGAG G CCAACCUC | 1728 | GAGGTTGG GGCTAGCTACAACGA CTCGATGA | 6525 |
| 7018 | CGAGGCCA A CCUCCUGU | 1729 | ACAGGAGG GGCTAGCTACAACGA TGGCCTCG | 6526 |
| 7025 | AACCUCCU G UGGCGGCA | 1730 | TGCCGCGA GGCTAGCTACAACGA AGGAGGTT | 6527 |
| 7028 | CUCCUGUG G CGGCAGGA | 1731 | TCCTGCCG GGCTAGCTACAACGA CACAGGAG | 6528 |
| 7031 | CUGUGGCG G CAGGAGAU | 1732 | ATCTCCTG GGCTAGCTACAACGA CGCCACAG | 6529 |
| 7038 | GGCAGGAG A UGGGCGGU | 1733 | ACCGCCGA GGCTAGCTACAACGA CTCCTGCC | 6530 |
| 7042 | GGAGAUGG G CGGUAACA | 1734 | TGTTACCG GGCTAGCTACAACGA CCATCTCC | 6531 |
| 7045 | GAUGGGCG G UAACAUCA | 1735 | TGATGTTA GGCTAGCTACAACGA CGCCCATC | 6532 |
| 7048 | GGGCGGUA A CAUCACUC | 1736 | GAGTGATG GGCTAGCTACAACGA TACCGCCC | 6533 |
| 7050 | GCGGUAAC A UCACUCGC | 1737 | GCGAGTGA GGCTAGCTACAACGA GTTACCGC | 6534 |
| 7053 | GUAACAUC A CUCGCGUG | 1738 | CACGCGAG GGCTAGCTACAACGA GATGTTAC | 6535 |
| 7057 | CAUCACUC G CGUGGAGU | 1739 | ACTCCACG GGCTAGCTACAACGA GAGTGATG | 6536 |
| 7059 | UCACUCGC G UGGAGUCA | 1740 | TGACTCGA GGCTAGCTACAACGA GCGAGTGA | 6537 |
| 7064 | CGCGUGGA G UCAGAGAA | 1741 | TTCTCTGA GGCTAGCTACAACGA TCCACGCG | 6538 |
| 7072 | GUCAGAGA A UAAGGUAG | 1742 | CTACCTTA GGCTAGCTACAACGA TCTCTGAC | 6539 |
| 7077 | AGAAUAAG G UAGUUACC | 1743 | GGTAACTA GGCTAGCTACAACGA CTTATTCT | 6540 |
| 7080 | AUAAGGUA G UUACCCUG | 1744 | CAGGGTAA GGCTAGCTACAACGA TACCTTAT | 6541 |
| 7083 | AGGUAGUU A CCCUGCAC | 1745 | GTCCAGGG GGCTAGCTACAACGA AACTACCT | 6542 |
| 7090 | UACCCUGG A CUCUUUUG | 1746 | CAAAAGAG GGCTAGCTACAACGA CCAGGGTA | 6543 |
| 7099 | CUCUUUUG A CCCGCUUC | 1747 | GAAGCGGG GGCTAGCTACAACGA CAAAAGAG | 6544 |
| 7103 | UUUGACCC G CUUCGAGC | 1748 | GCTCCAAG GGCTAGCTACAACGA GGGTCAAA | 6545 |
| 7110 | CGCUUCGA G CGGAGGAG | 1749 | CTCCTCCG GGCTAGCTACAACGA TCGAAGCG | 6546 |
| 7120 | CGACCACG A UGAGAGAG | 1750 | CTCTCTCA GGCTAGCTACAACGA CCTCCTCC | 6547 |
| 7131 | AGAGAGAG G UGUCCAUU | 1751 | AATGGACA GGCTAGCTACAACGA CTCTCTCT | 6548 |
| 7133 | AGAGAGGU G UCCAUUCC | 1752 | GGAATGGA GGCTAGCTACAACGA ACCTCTCT | 6549 |
| 7137 | ACCUGUCC A UUCCGCCG | 1753 | CGCCGGAA GGCTAGCTACAACGA GCACACCT | 6550 |
| 7143 | CCAUUCCG G CCCAGAUC | 1754 | GATCTCCG GGCTAGCTACAACGA CGGAATGG | 6551 |
| 7149 | CGGCGGAG A UCCUGCGG | 1755 | CCGCAGGA GGCTAGCTACAACGA CTCCGCCG | 6552 |
| 7154 | GAGAUCCU G CGGAAAUC | 1756 | GATTTCCG GGCTAGCTACAACGA AGGATCTC | 6553 |
| 7160 | CUGCGGAA A UCCAAGAA | 1757 | TTCTTGCA GGCTAGCTACAACGA TTCCGCAG | 6554 |
| 7169 | UCCAAGAA G UUUCCUUC | 1758 | GAAGGAAA GGCTAGCTACAACGA TTCTTGGA | 6555 |
| 7179 | UUCCUUCA G CGUUACCC | 1759 | CGCTAACG GGCTAGCTACAACGA TGAACGAA | 6556 |
| 7181 | CCUUCAGC G UUACCCAU | 1760 | ATGGGTAA GGCTAGCTACAACGA GCTGAACG | 6557 |
| 7184 | UCAGCGUU A CCCAUAUG | 1761 | CATATGGG GGCTAGCTACAACGA AACGCTGA | 6558 |
| 7188 | CCUUACCC A UAUGGGCA | 1762 | TGCCCATA GGCTAGCTACAACGA GGGTAACG | 6559 |
| 7190 | UUACCCAU A UGCGCACG | 1763 | CGTGCCGA GGCTAGCTACAACGA ATGGGTAA | 6560 |
| 7194 | CCAUAUGG G CACGCCCG | 1764 | CGGGCGTG GGCTAGCTACAACGA CCATATGG | 6561 |
| 7196 | AUAUGGGC A CGCCCGGA | 1765 | TCCGGGCG GGCTAGCTACAACGA GCCCATAT | 6562 |
| 7198 | AUGGGCAC G CCCGGAUU | 1766 | AATCCGGG GGCTAGCTACAACGA GTGCCCAT | 6563 |
| 7204 | ACGCCCGG A UUACAACC | 1767 | GGTTGTAA GGCTAGCTACAACGA CCGGGCGT | 6564 |
| 7207 | CCCGGAUU A CAACCCUC | 1768 | GAGGGTTG GGCTAGCTACAACGA AATCCGGG | 6565 |
| 7210 | GGAUUACA A CCCUCCAC | 1769 | GTGGAGGG GGCTAGCTACAACGA TGTAATCC | 6566 |
| 7217 | AACCCUCC A CUACUAGA | 1770 | TCTAGTAG GGCTAGCTACAACGA GGAGGGTT | 6567 |
| 7220 | CCUCCACU A CUAGAGCC | 1771 | GGCTCTAG GGCTAGCTACAACGA AGTGGAGG | 6568 |
| 7226 | CUACUAGA G CCCUGGAA | 1772 | TTCCAGGG GGCTAGCTACAACGA TCTAGTAG | 6569 |
| 7237 | CUGGAAAG A CCCAGACU | 1773 | AGTCTGGG GGCTAGCTACAACGA CTTTCCAG | 6570 |
| 7243 | AGACCCAG A CUACGUCC | 1774 | GGACGTAG GGCTAGCTACAACGA CTGGGTCT | 6571 |
| 7246 | CCCAGACU A CGUCCCUC | 1775 | GAGGGACG GGCTAGCTACAACGA AGTCTGGG | 6572 |
| 7248 | CAGACUAC G UCCCUCCG | 1776 | CGGAGGGA GGCTAGCTACAACGA GTAGTCTG | 6573 |
| 7257 | UCCCUCCG G UGGUACAC | 1777 | GTGTACGA GGCTAGCTACAACGA CGGAGGGA | 6574 |
| 7260 | CUCCGGUG G UACACGGG | 1778 | CCCGTGTA GGCTAGCTACAACGA CACCGGAG | 6575 |
| 7262 | CCGGUGGU A CACGGGUG | 1779 | CACCCGTG GGCTAGCTACAACGA ACCACCGG | 6576 |
| 7264 | GGUGGUAC A CGGGUGCC | 1780 | GGCACCCG GGCTAGCTACAACGA GTACCACC | 6577 |
| 7268 | GUACACGG G UGCCCAUU | 1781 | AATGGGCA GGCTAGCTACAACGA CCGTGTAC | 6578 |
| 7270 | ACACGGGU G CCCAUUGC | 1782 | GCAATGGG GGCTAGCTACAACGA ACCCGTGT | 6579 |
| 7274 | GGGUGCCC A UUGCCACC | 1783 | GGTGGCAA GGCTAGCTACAACGA GGGCACCC | 6580 |
| 7277 | UGCCCAUU G CCACCUGC | 1784 | GCAGGTGG GGCTAGCTACAACGA AATGGGCA | 6581 |
| 7280 | CCAUUGCC A CCUGCCAA | 1785 | TTGGCAGG GGCTAGCTACAACGA GGCAATGG | 6582 |
| 7284 | UGCCACCU G CCAAGGCC | 1786 | GGCCTTGG GGCTAGCTACAACGA AGGTGGCA | 6583 |
| 7290 | CUGCCAAG G CCCCUCGA | 1787 | TGGAGGGG GGCTAGCTACAACGA CTTGGCAG | 6584 |
| 7299 | CCCCUCCA A UACCACCU | 1788 | AGGTGGTA GGCTAGCTACAACGA TGGAGGGG | 6585 |
| 7301 | CCUCCAAU A CCACCUCC | 1789 | GGAGGTGG GGCTAGCTACAACGA ATTGGAGG | 6586 |
| 7304 | CCAAUACC A CCUCCACG | 1790 | CGTGGAGG GGCTAGCTACAACGA GGTATTGG | 6587 |
| 7310 | CCACCUCC A CGGAGGAA | 1791 | TTCCTCCG GGCTAGCTACAACGA GGAGGTGG | 6588 |
| 7323 | GGAAGAGG A CGGUUGUU | 1792 | AACAACCG GGCTAGCTACAACGA CCTCTTCC | 6589 |
| 7326 | AGAGGACG G UUGUUCUG | 1793 | CAGAACAA GGCTAGCTACAACGA CGTCCTCT | 6590 |
| 7329 | GGACGGUU G UUCUGACA | 1794 | TGTCAGAA GGCTAGCTACAACGA AACCGTCC | 6591 |
| 7335 | UUGUUCUG A CAGAGUCC | 1795 | GGACTCTG GGCTAGCTACAACGA CAGAACAA | 6592 |
| 7340 | CUGACAGA G UCCACCGU | 1796 | ACGGTGGA GGCTAGCTACAACGA TCTGTCAG | 6593 |
| 7344 | CAGAGUCC A CCGUGUCU | 1797 | AGACACGG GGCTAGCTACAACGA GGACTCTG | 6594 |
| 7347 | AGUCCACC G UGUCUUCU | 1798 | AGAAGACA GGCTAGCTACAACGA GGTGGACT | 6595 |
| 7349 | UCCACCGU G UCUUCUGC | 1799 | GCAGAAGA GGCTAGCTACAACGA ACGGTGGA | 6596 |
| 7356 | UGUCUUCU G CCUUGGCG | 1800 | CGCCAAGG GGCTAGCTACAACGA AGAAGACA | 6597 |
| 7362 | CUGCCUUG G CGGAGCUC | 1801 | GAGCTCCG GGCTAGCTACAACGA CAAGGCAG | 6598 |
| 7367 | UUGGCGGA G CUCGCCAC | 1802 | GTGGCGAG GGCTAGCTACAACGA TCCGCCAA | 6599 |
| 7371 | CGGAGCUC G CCACAAAG | 1803 | CTTTGTGG GGCTAGCTACAACGA GAGCTCCG | 6600 |
| 7374 | AGCUCGCC A CAAAGACC | 1804 | GGTCTTTG GGCTAGCTACAACGA GGCGAGCT | 6601 |
| 7380 | CCACAAAG A CCUUCGGC | 1805 | GCCGAAGG GGCTAGCTACAACGA CTTTGTGG | 6602 |
| 7387 | GACCUUCG G CACCUCUG | 1806 | CAGAGCTG GGCTAGCTACAACGA CGAAGGTC | 6603 |
| 7390 | CUUCGGCA G CUCUGAAU | 1807 | ATTCAGAG GGCTAGCTACAACGA TGCCGAAG | 6604 |
| 7397 | AGCUCUGA A UCAUCGGC | 1808 | GCCGATGA GGCTAGCTACAACGA TCAGAGCT | 6605 |
| 7400 | UCUGAAUC A UCGGCCGC | 1809 | GCGGCCGA GGCTAGCTACAACGA GATTCAGA | 6606 |
| 7404 | AAUCAUCG G CCGCUGAU | 1810 | ATCAGCGG GGCTAGCTACAACGA CGATGATT | 6607 |
| 7407 | CAUCGGCC G CUGAUAGA | 1811 | TCTATCAG GGCTAGCTACAACGA GGCCGATG | 6608 |
| 7411 | GGCCGCUG A UAGAGGUA | 1812 | TACCTCTA GGCTAGCTACAACGA CAGCGGCC | 6609 |
| 7417 | UGAUAGAG G UACGGCAA | 1813 | TTGCCGTA GGCTAGCTACAACGA CTCTATCA | 6610 |
| 7419 | AUAGAGGU A CGGCAACC | 1814 | GGTTGCCG GGCTAGCTACAACGA ACCTCTAT | 6611 |
| 7422 | GAGGUACG G CAACCGCC | 1815 | GGCGGTTG GGCTAGCTACAACGA CGTACCTC | 6612 |
| 7425 | GUACGGCA A CCGCCCCC | 1816 | GGGGGCCG GGCTAGCTACAACGA TGCCGTAC | 6613 |
| 7428 | CGGCAACC G CCCCCCCC | 1817 | GGCGGGCG GGCTAGCTACAACGA GGTTGCCC | 6614 |
| 7438 | CCCCCCCG A CCAGACCU | 1818 | ACGTCTGG GGCTAGCTACAACGA CGGGGGGG | 6615 |
| 7443 | CCGACCAG A CCUCCAAU | 1819 | ATTGGAGG GGCTAGCTACAACGA CTGGTCGG | 6616 |
| 7450 | GACCUCCA A UGACGGUG | 1820 | CACCGTCA GGCTAGCTACAACGA TGGAGCTC | 6617 |
| 7453 | CUCCAAUG A CGGUGACG | 1821 | CGTCACCG GGCTAGCTACAACGA CATTGGAG | 6618 |
| 7456 | CAAUGACG G UGACGCAG | 1822 | CTGCGTCA GGCTAGCTACAACGA CGTCATTG | 6619 |
| 7459 | UGACGGUG A CGCAGGAU | 1823 | ATCCTGCG GGCTAGCTACAACGA CACCGTCA | 6620 |
| 7461 | ACGGUGAC G CAGGAUCC | 1824 | GGATCCTG GGCTAGCTACAACGA GTCACCGT | 6621 |
| 7466 | GACGCAGG A UCCGACGU | 1825 | ACGTCGGA GGCTAGCTACAACGA CCTGCGTC | 6622 |
| 7471 | AGGAUCCG A CGUUGAGU | 1826 | ACTCAACG GGCTAGCTACAACGA CGGATCCT | 6623 |
| 7473 | GAUCCGAC G UUGAGUCG | 1827 | CGACTCAA GGCTAGCTACAACGA GTCGGATC | 6624 |
| 7478 | GACGUUGA G UCGUACUC | 1828 | GAGTACGA GGCTAGCTACAACGA TCAACGTC | 6625 |
| 7481 | GUUGAGUC G UACUCCUC | 1829 | GAGGAGTA GGCTAGCTACAACGA GACTCAAC | 6626 |
| 7483 | UGAGUCGU A CUCCUCUA | 1830 | TAGAGGAG GGCTAGCTACAACGA ACGACTCA | 6627 |
| 7491 | ACUCCUCU A UGCCCCCC | 1831 | GGGGGGCA GGCTAGCTACAACGA AGAGGACT | 6628 |
| 7493 | UCCUCUAU G CCCCCCCU | 1832 | AGGGGGGG GGCTAGCTACAACGA ATAGAGGA | 6629 |
| 7511 | GAGGGGGA G CCGGGGGA | 1833 | TCCCCCGG GGCTAGCTACAACGA TCCCCCTC | 6630 |
| 7519 | GCCGGGGG A UCCCGAUC | 1834 | GATCGGGA GGCTAGCTACAACGA CCCCCGGC | 6631 |
| 7525 | GGAUCCCG A UCUCAGCG | 1835 | CGCTGAGA GGCTAGCTACAACGA CGGGATCC | 6632 |
| 7531 | CGAUCUCA G CGACGGGU | 1836 | ACCCGTCG GGCTAGCTACAACGA TGAGATCG | 6633 |
| 7534 | UCUCAGCG A CGGGUCUU | 1837 | AAGACCCG GGCTAGCTACAACGA CGCTGAGA | 6634 |
| 7538 | AGCGACGG G UCUUGGUC | 1838 | GACCAAGA GGCTAGCTACAACGA CCGTCGCT | 6635 |
| 7544 | GGGUCUUG G UCUACCGU | 1839 | ACGGTAGA GGCTAGCTACAACGA CAAGACCC | 6636 |
| 7548 | CUUGGUCU A CCGUGAGC | 1840 | GCTCACGG GGCTAGCTACAACGA AGACCAAG | 6637 |
| 7551 | GGUCUACC G UGAGCGAA | 1841 | TTCGCTCA GGCTAGCTACAACGA GGTAGACC | 6638 |
| 7555 | UACCGUGA G CGAAGAGG | 1842 | CCTCTTCG GGCTAGCTACAACGA TCACGGTA | 6639 |
| 7563 | GCGAAGAG G CUGGCGAG | 1843 | CTCGCCAG GGCTAGCTACAACGA CTCTTCGC | 6640 |
| 7567 | AGAGGCUG G CGAGGAUG | 1844 | CATCCTCG GGCTAGCTACAACGA CAGCCTCT | 6641 |
| 7573 | UGGCGAGG A UGUCGUCU | 1845 | AGACGACA GGCTAGCTACAACGA CCTCGCCA | 6642 |
| 7575 | GCGAGGAU G UCGUCUGC | 1846 | CCAGACGA GGCTAGCTACAACGA ATCCTCGC | 6643 |
| 7578 | AGGAUGUC G UCUGCUGC | 1847 | GCAGCAGA GGCTAGCTACAACGA GACATCCT | 6644 |
| 7582 | UGUCGUCU G CUGCUCGA | 1848 | TCGAGCAG GGCTAGCTACAACGA AGACGACA | 6645 |
| 7585 | CGUCUGCU G CUCGAUGU | 1849 | ACATCGAG GGCTAGCTACAACGA AGCAGACG | 6646 |
| 7590 | GCUGCUCG A UGUCCUAC | 1850 | GTAGGACA GGCTAGCTACAACGA CGAGCAGC | 6647 |
| 7592 | UGCUCGAU G UCCUACAC | 1851 | GTGTAGGA GGCTAGCTACAACGA ATCGAGCA | 6648 |
| 7597 | GAUGUCCU A CACAUGGA | 1852 | TCCATGTG GGCTAGCTACAACGA AGGACATC | 6649 |
| 7599 | UGUCCUAC A CAUGGACG | 1853 | CGTCCATG GGCTAGCTACAACGA GTAGGACA | 6650 |
| 7601 | UCCUACAC A UGGACGGG | 1854 | CCCGTCGA GGCTAGCTACAACGA GTGTAGGA | 6651 |
| 7605 | ACACAUGG A CGGGCGCC | 1855 | GGCGCCCG GGCTAGCTACAACGA CCATGTGT | 6652 |
| 7609 | AUGGACGG G CGCCCUGA | 1856 | TCAGGGCG GGCTAGCTACAACGA CCGTCCAT | 6653 |
| 7611 | GGACGGGC G CCCUGAUC | 1857 | GATCAGGG GGCTAGCTACAACGA GCCCGTCC | 6654 |
| 7617 | GCGCCCUG A UCACGCGA | 1858 | TGGCGTGA GGCTAGCTACAACGA CAGGGCGC | 6655 |
| 7620 | CCCUGAUC A CGCCAUGC | 1859 | GCATGGCG GGCTAGCTACAACGA GATCAGGG | 6656 |
| 7622 | CUGAUCAC G CCAUGCGC | 1860 | GCGCATGG GGCTAGCTACAACGA GTGATCAG | 6657 |
| 7625 | AUCACGCC A UGCGCUGC | 1861 | GCAGCGCA GGCTAGCTACAACGA GGCGTGAT | 6658 |
| 7627 | CACGCCAU G CGCUGCGG | 1862 | CCGCAGCG GGCTAGCTACAACGA ATGGCGTG | 6659 |
| 7629 | CGCCAUGC G CUGCGGAG | 1863 | CTCCGCAG GGCTAGCTACAACGA GCATGGCG | 6660 |
| 7632 | CAUGCGCU G CGGAGGAA | 1864 | TTCCTCCG GGCTAGCTACAACGA AGCGCATG | 6661 |
| 7642 | GGAGGAAA G CAAGUUGC | 1865 | GCAACTTG GGCTAGCTACAACGA TTTCCTCC | 6662 |
| 7646 | GAAAGCAA G UUGCCCAU | 1866 | ATGGGCAA GGCTAGCTACAACGA TTGCTTTC | 6663 |
| 7649 | AGCAAGUU G CCCAUCAA | 1867 | TTGATGGG GGCTAGCTACAACGA AACTTGCT | 6664 |
| 7653 | AGUUGCCC A UCAACGCG | 1868 | CGCGTTGA GGCTAGCTACAACGA GGGCAACT | 6665 |
| 7657 | GCCCAUCA A CGCGUUGA | 1869 | TCAACGCG GGCTAGCTACAACGA TGATGGGC | 6666 |
| 7659 | CCAUCAAC G CGUUGAGC | 1870 | GCTCAACG GGCTAGCTACAACGA CTTGATGC | 6667 |
| 7661 | AUCAACGC G UUGAGCAA | 1871 | TTGCTCAA GGCTAGCTACAACGA GCGTTGAT | 6668 |
| 7666 | CGCGUUGA G CAACUCUU | 1872 | AAGAGTTG GGCTAGCTACAACGA TCAACGCG | 6669 |
| 7669 | GUCGAGCA A CUCUUUCC | 1873 | GCAAACAG GGCTAGCTACAACGA TGCTCAAC | 6670 |
| 7676 | AACUCUUU G CUCCCUCA | 1874 | TGACCCAG GGCTAGCTACAACGA AAAGAGTT | 6671 |
| 7679 | UCUUUGCU G CGUCACGA | 1875 | TGCTCACG GGCTAGCTACAACGA AGCAAACA | 6672 |
| 7681 | UUUCCUGC G UCACCACA | 1876 | TCTGCTCA GGCTAGCTACAACGA GCAGCAAA | 6673 |
| 7684 | CCUCCCUC A CCACAACA | 1877 | TGTTCTCG GGCTAGCTACAACGA CACCCACC | 6674 |
| 7687 | GCGUCACC A CAACAUGG | 1878 | CCATGTTG GGCTAGCTACAACGA GGTGACCC | 6675 |
| 7690 | UCACCACA A CAUGGUCU | 1879 | AGACCATG GGCTAGCTACAACGA TGTGGTGA | 6676 |
| 7692 | ACCACAAC A UGGUCUAC | 1880 | GTAGACGA GGCTAGCTACAACGA GTTGTGGT | 6677 |
| 7695 | ACAACAUG G UCUACGCU | 1881 | AGCGTAGA GGCTAGCTACAACGA CATGTTGT | 6678 |
| 7699 | CAUGGUCU A CGCUACAA | 1882 | TTGTAGCG GGCTAGCTACAACGA AGACCATG | 6679 |
| 7701 | UGGUCUAC G CUACAACA | 1883 | TGTTGTAG GGCTAGCTACAACGA GTAGACCA | 6680 |
| 7704 | UCUACGCU A CAACAUCU | 1884 | AGATGTTG GGCTAGCTACAACGA AGCGTAGA | 6681 |
| 7707 | ACGCUACA A CAUCUCGC | 1885 | GCGAGATG GGCTAGCTACAACGA TGTAGCGT | 6682 |
| 7709 | GCUACAAC A UCUCGCAG | 1886 | CTGCGAGA GGCTAGCTACAACGA GTTGTAGC | 6683 |
| 7714 | AACAUCUC G CAGCGCAA | 1887 | TTGCGCTG GGCTAGCTACAACGA GAGATGTT | 6684 |
| 7717 | AUCUCGCA G CGGAAGCC | 1888 | GGCTTGCG GGCTAGCTACAACGA TGCGAGAT | 6685 |
| 7719 | CUCGCAGC G GAAGCCAG | 1889 | CTGGCTTG GGCTAGCTACAACGA GCTGCGAG | 6686 |
| 7723 | CAGCGCAA G CCAGCGGC | 1890 | GCCGCTGG GGCTAGCTACAACGA TTGCGCTG | 6687 |
| 7727 | GCAAGCGA G CGGCAGAA | 1891 | TTCTGCCG GGCTAGCTACAACGA TGGCTTGC | 6688 |
| 7730 | AGCCAGCG G CAGAAGAA | 1892 | TTCTTCTG GGCTAGCTACAACGA CGCTGGCT | 6689 |
| 7740 | AGAAGAAG G UCACCUUU | 1893 | AAAGGTGA GGCTAGCTACAACGA CTTCTTCT | 6690 |
| 7743 | AGAAGGUC A CCUUUGAC | 1894 | GTCAAAGG GGCTAGCTACAACGA GACCTTCT | 6691 |
| 7750 | CACCUUUG A CAGACUGC | 1895 | GCAGTCTG GGCTAGCTACAACGA CAAAGGTG | 6692 |
| 7754 | UUUGACAG A CUGCAAGU | 1896 | ACTTGCAG GGCTAGCTACAACGA CTGTCAAA | 6693 |
| 7757 | GACAGACU G CAAGUCCU | 1897 | AGGACTTG GGCTAGCTACAACGA AGTCTGTC | 6694 |
| 7761 | GACUGCAA G UCCUGGAC | 1898 | GTCCAGGA GGCTAGCTACAACGA TTGCAGTC | 6695 |
| 7768 | AGUCCUGG A CGACCACU | 1899 | AGTGGTCG GGCTAGCTACAACGA CCAGGACT | 6696 |
| 7771 | CCUGGACG A CCACUACC | 1900 | GGTAGTGG GGCTAGCTACAACGA CGTCCAGG | 6697 |
| 7774 | GGACGACC A CUACCGGG | 1901 | CCCGGTAG GGCTAGCTACAACGA GGTCGTCC | 6698 |
| 7777 | CGACCACU A CCGGGACG | 1902 | CGTCCCGG GGCTAGCTACAACGA AGTGGTCG | 6699 |
| 7783 | CUACCGGG A CGUGCUCA | 1903 | TGAGCACG GGCTAGCTACAACGA CCCGGTAG | 6700 |
| 7785 | ACCGGGAC G UGCUCAAG | 1904 | CTTGAGCA GGCTAGCTACAACGA GTCCCGGT | 6701 |
| 7787 | CGGGACGU G CUCAAGGA | 1905 | TCCTTGAG GGCTAGCTACAACGA ACGTCCCG | 6702 |
| 7797 | UCAAGGAG A UGAAGGCG | 1906 | CGCCTTCA GGCTAGCTACAACGA CTCCTTGA | 6703 |
| 7803 | AGAUGAAG G CGAAGGCG | 1907 | CGCCTTCG GGCTAGCTACAACGA CTTCATCT | 6704 |
| 7809 | AGGCGAAG G CGUCCACA | 1908 | TGTGGACG GGCTAGCTACAACGA CTTCGCCT | 6705 |
| 7811 | GCGAAGGC G UCCACAGU | 1909 | ACTGTGGA GGCTAGCTACAACGA GCCTTCGC | 6706 |
| 7815 | AGGCGUCC A CAGUUAAG | 1910 | CTTAACTG GGCTAGCTACAACGA GGACGCCT | 6707 |
| 7818 | CGUCCACA G UUAAGGCU | 1911 | AGCCTTAA GGCTAGCTACAACGA TGTGGACG | 6708 |
| 7824 | CAGUUAAG G CUAAACUU | 1912 | AAGTTTAG GGCTAGCTACAACGA CTTAACTG | 6709 |
| 7829 | AAGGCUAA A CUUCUAUC | 1913 | GATAGAAG GGCTAGCTACAACGA TTAGCCTT | 6710 |
| 7835 | AAACUUCU A UCCGUAGA | 1914 | TCTACGGA GGCTAGCTACAACGA AGAAGTTT | 6711 |
| 7839 | UUCUAUCC G UAGAGGAA | 1915 | TTCCTCTA GGCTAGCTACAACGA GGATAGAA | 6712 |
| 7848 | UAGAGGAA G CCUGCAGA | 1916 | TCTGCAGG GGCTAGCTACAACGA TTCCTCTA | 6713 |
| 7852 | GGAAGCCU G CAGACUGA | 1917 | TCAGTCTG GGCTAGCTACAACGA AGGCTTCC | 6714 |
| 7856 | GCCUGCAG A CUGACGCC | 1918 | GGCGTCAG GGCTAGCTACAACGA CTGCAGGC | 6715 |
| 7860 | GCAGACUG A CGCCCCGA | 1919 | TGGGGGCG GGCTAGCTACAACGA CAGTCTGC | 6716 |
| 7862 | AGACUGAC G CCCCCACA | 1920 | TGTGGGGG GGCTAGCTACAACGA GTCAGTCT | 6717 |
| 7868 | ACGCCCCC A CAUUCGGC | 1921 | GCCGAATG GGCTAGCTACAACGA GGGGGCGT | 6718 |
| 7870 | GCCCCCAC A UUCGGCGA | 1922 | TGGCCGAA GGCTAGCTACAACGA GTGGGGGC | 6719 |
| 7875 | CACAUUCG G CCAGGUCC | 1923 | GGACCTGG GGCTAGCTACAACGA CGAATGTG | 6720 |
| 7880 | UCCGCCAG G UCCAAAUU | 1924 | AATTTGGA GGCTAGCTACAACGA CTGGCCGA | 6721 |
| 7886 | AGGUCCAA A UUUGGUUA | 1925 | TAACCAAA GGCTAGCTACAACGA TTGGACCT | 6722 |
| 7891 | CAAAUUUG G UUAUGGGG | 1926 | CCCCATAA GGCTAGCTACAACGA CAAATTTG | 6723 |
| 7894 | AUUUGGUU A UGGGGCAA | 1927 | TTGCCCGA GGCTAGCTACAACGA AACCAAAT | 6724 |
| 7899 | GUUAUGGG G CAAAGGAC | 1928 | GTCCTTTG GGCTAGCTACAACGA CCCATAAC | 6725 |
| 7906 | GGCAAAGG A CGUCCGGA | 1929 | TCCGGACG GGCTAGCTACAACGA CCTTTGCC | 6726 |
| 7908 | CAAAGGAC G UCCGGAAC | 1930 | GTTCCGGA GGCTAGCTACAACGA GTCCTTTG | 6727 |
| 7915 | CGUCCGGA A CCUAUCGA | 1931 | TGGATAGG GGCTAGCTACAACGA TCCGGACG | 6728 |
| 7919 | CGGAACCU A UCCAGCGG | 1932 | CCGCTGGA GGCTAGCTACAACGA AGGTTCCG | 6729 |
| 7924 | CCUAUCGA G CGGGGCCG | 1933 | CGGCCCCG GGCTAGCTACAACGA TGGATAGG | 6730 |
| 7929 | CCAGCGGG G CCGUCAAC | 1934 | GTTGACGG GGCTAGCTACAACGA CCCGCTGG | 6731 |
| 7932 | GCGGGGCC G UCAACCAC | 1935 | GTGGTTGA GGCTAGCTACAACGA GGCCCCGC | 6732 |
| 7936 | GGCCGUCA A CCACAUCC | 1936 | GGATGTGG GGCTAGCTACAACGA TGACGGCC | 6733 |
| 7939 | CGUCAACC A CAUCCGCU | 1937 | AGCGGATG GGCTAGCTACAACGA GGTTGACG | 6734 |
| 7941 | UCAACCAC A UCCGCUCC | 1938 | GGAGCGGA GGCTAGCTACAACGA GTGGTTGA | 6735 |
| 7945 | CCACAUCC G CUCCGUGU | 1939 | ACACGGAG GGCTAGCTACAACGA GGATGTGG | 6736 |
| 7950 | UCCGCUCC G UGUGGAAG | 1940 | CTTCCACA GGCTAGCTACAACGA GGAGCGGA | 6737 |
| 7952 | CGCUCCGU G UGGAAGGA | 1941 | TCCTTCGA GGCTAGCTACAACGA ACGGAGCG | 6738 |
| 7960 | GUGGAAGG A CUUGCUGG | 1942 | CCAGCAAG GGCTAGCTACAACGA CCTTCCAC | 6739 |
| 7964 | AAGGACUU G CUGGAAGA | 1943 | TCTTCCAG GGCTAGCTACAACGA AAGTCCTT | 6740 |
| 7972 | GCUGGAAG A CACUGAGA | 1944 | TCTCAGTG GGCTAGCTACAACGA CTTCCAGC | 6741 |
| 7974 | UGGAAGAC A CUGAGACA | 1945 | TGTCTCAG GGCTAGCTACAACGA GTCTTCCA | 6742 |
| 7980 | ACACUGAG A CACCAAUU | 1946 | AATTGGTG GGCTAGCTACAACGA CTCAGTGT | 6743 |
| 7982 | ACUGAGAC A CCAAUUGA | 1947 | TCAATTGG GGCTAGCTACAACGA GTCTCAGT | 6744 |
| 7986 | AGACACCA A UUGAUACC | 1948 | GGTATCAA GGCTAGCTACAACGA TGGTGTCT | 6745 |
| 7990 | ACCAAUUG A UACCACGA | 1949 | TGGTGGTA GGCTAGCTACAACGA CAATTGGT | 6746 |
| 7992 | CAAUUGAU A CCACCAUC | 1950 | GATGGTGG GGCTAGCTACAACGA ATCAATTG | 6747 |
| 7995 | UUGAUACC A CCAUCAUG | 1951 | CATGATGG GGCTAGCTACAACGA GGTATCAA | 6748 |
| 7998 | AUACCACC A UCAUGGCA | 1952 | TGCCATGA GGCTAGCTACAACGA GGTGGTAT | 6749 |
| 8001 | CCACCAUC A UGGCAAAA | 1953 | TTTTGCGA GGCTAGCTACAACGA GATGGTGG | 6750 |
| 8004 | CCAUCAUG G CAAAAAAU | 1954 | ATTTTTTG GGCTAGCTACAACGA CATGATGG | 6751 |
| 8011 | GGCAAAAA A UGAGGUUU | 1955 | AAACCTCA GGCTAGCTACAACGA TTTTTGCC | 6752 |
| 8016 | AAAAUGAG G UUUUCUGC | 1956 | GCAGAAAA GGCTAGCTACAACGA CTCATTTT | 6753 |
| 8023 | GGUUUUCU G CGUCCAAC | 1957 | GTTGGACG GGCTAGCTACAACGA AGAAAACC | 6754 |
| 8025 | UUUUCUGC G UCCAACGA | 1958 | TGGTTGGA GGCTAGCTACAACGA GCAGAAAA | 6755 |
| 8030 | UGCGUCCA A CCAGAGAA | 1959 | TTCTCTGG GGCTAGCTACAACGA TGGACGCA | 6756 |
| 8044 | GAAAGGAG G CCGCAAGC | 1960 | GCTTGCGG GGCTAGCTACAACGA CTCCTTTC | 6757 |
| 8047 | AGGAGGCC G CAAGCCAG | 1961 | CTGGCTTG GGCTAGCTACAACGA GGCCTCCT | 6758 |
| 8051 | GGCCGCAA G CCAGCUCG | 1962 | CGAGCTGG GGCTAGCTACAACGA TTGCGGCC | 6759 |
| 8055 | GCAAGCGA G CUCGCCUU | 1963 | AAGGCGAG GGCTAGCTACAACGA TGGCTTGC | 6760 |
| 8059 | GCCAGCUC G CCUUAUCG | 1964 | CGATAAGG GGCTAGCTACAACGA GAGCTGGC | 6761 |
| 8064 | CUCGCCUU A UCGUGUUC | 1965 | GAACACGA GGCTAGCTACAACGA AAGGCGAG | 6762 |
| 8067 | GCCUUAUC G UGUUCCCA | 1966 | TGGGAACA GGCTAGCTACAACGA GATAAGGC | 6763 |
| 8069 | CUUAUCGU G UUCCCAGA | 1967 | TCTGGGAA GGCTAGCTACAACGA ACGATAAG | 6764 |
| 8077 | GUUCCCAG A CUUGGGGG | 1968 | CCCCCAAG GGCTAGCTACAACGA CTGGGAAC | 6765 |
| 8085 | ACUUGGGG G UUCGUGUG | 1969 | CACACGAA GGCTAGCTACAACGA CCCCAAGT | 6766 |
| 8089 | GGGGGUUC G UGUGUGCG | 1970 | CGCACACA GGCTAGCTACAACGA GAACCCCC | 6767 |
| 8091 | GGGUUCGU G UGUGCGAG | 1971 | CTCGCACA GGCTAGCTACAACGA ACGAACCC | 6768 |
| 8093 | GUUCGUGU G UGCGAGAA | 1972 | TTCTCGCA GGCTAGCTACAACGA ACACGAAC | 6769 |
| 8095 | UCGUGUGU G CGAGAAAA | 1973 | TTTTCTCG GGCTAGCTACAACGA ACACACCA | 6770 |
| 8103 | GCGAGAAA A UGGCCCUU | 1974 | AAGGGCGA GGCTAGCTACAACGA TTTCTCGC | 6771 |
| 8106 | AGAAAAUG G CCCUUUAC | 1975 | GTAAAGGG GGCTAGCTACAACGA CATTTTCT | 6772 |
| 8113 | GGCCCUUU A CGACGUCG | 1976 | CCACGTCG GGCTAGCTACAACGA AAAGGGCC | 6773 |
| 8116 | CCUUUACG A CGUGGUCU | 1977 | AGACCACG GGCTAGCTACAACGA CGTAAAGG | 6774 |
| 8118 | UUUACGAC G UGGUCUCC | 1978 | GGAGACGA GGCTAGCTACAACGA CTCGTAAA | 6775 |
| 8121 | ACCACCUG G UCUCCACC | 1979 | GGTGGAGA GGCTAGCTACAACGA CACGTCGT | 6776 |
| 8127 | UCGUCUCC A CCCUUCCU | 1980 | AGGAAGGG GGCTAGCTACAACGA GGAGACCA | 6777 |
| 8139 | UUCCUCAG G CCGUGAUG | 1981 | CATCACGG GGCTAGCTACAACGA CTGAGGAA | 6778 |
| 8142 | CUCACCCC G UGAUGGCC | 1982 | CCCCATCA GGCTAGCTACAACGA CCCCTCAC | 6779 |
| 8148 | ACCCCCUC A UCGCCUCU | 1983 | ACACCCGA GGCTAGCTACAACGA CACCCCCT | 6780 |
| 8149 | CGUCAUGG G CUCUUCAU | 1984 | ATGAAGAG GGCTAGCTACAACGA CCATCACG | 6781 |
| 8156 | GGCUCUUC A UACGGAUU | 1985 | AATCCGTA GGCTAGCTACAACGA GAAGAGCC | 6782 |
| 8158 | CUCUUCAU A CGCAUUCC | 1986 | GCAATCCG GGCTAGCTACAACGA ATCAAGAG | 6783 |
| 8162 | UCAUACGG A UUCCACUA | 1987 | TACTGGAA GGCTAGCTACAACGA CCGTATGA | 6784 |
| 8168 | GGAUUCGA G UACUCUCC | 1988 | CCACACTA GGCTAGCTACAACGA TGCAATCC | 6785 |
| 8170 | AUUCCAGU A CUCUCCUC | 1989 | CAGGAGAC GGCTAGCTACAACGA ACTGGAAT | 6786 |
| 8180 | UCUCCUCG G CAGCCCCU | 1990 | ACCCCCTC GGCTAGCTACAACGA CCACCAGA | 6787 |
| 8183 | CCUCCCGA G CGGGUUGA | 1991 | TCAACCCG GGCTAGCTACAACGA TCCCCACC | 6788 |
| 8187 | GGCAGCGG G UUGAGUUC | 1992 | GAACTCAA GGCTAGCTACAACGA CCGCTGCC | 6789 |
| 8192 | CGGGUUGA G UUCCUGGU | 1993 | ACCAGGAA GGCTAGCTACAACGA TCAACCCG | 6790 |
| 8199 | AGUUCCUG G UGAAUGCC | 1994 | GGCATTCA GGCTAGCTACAACGA CAGGAACT | 6791 |
| 8203 | CCUGGUGA A UGCCUGGA | 1995 | TCCAGGCA GGCTAGCTACAACGA TCACCAGG | 6792 |
| 8205 | UGGUGAAU G CCUGGAAA | 1996 | TTTCCAGG GGCTAGCTACAACGA ATTCACCA | 6793 |
| 8213 | GCCUGGAA A UCAAAGAA | 1997 | TTCTTTGA GGCTAGCTACAACGA TTCCAGGC | 6794 |
| 8222 | UCAAAGAA A UGCCCUAU | 1998 | ATAGGGCA GGCTAGCTACAACGA TTCTTTGA | 6795 |
| 8224 | AAAGAAAU G CCCUAUGG | 1999 | CCATAGGG GGCTAGCTACAACGA ATTTCTTT | 6796 |
| 8229 | AAUGCCCU A UGGGCUUU | 2000 | AAAGCCGA GGCTAGCTACAACGA AGGGCATT | 6797 |
| 8233 | CCCUAUGG G CUUUGCAU | 2001 | ATGCAAAG GGCTAGCTACAACGA CCATAGGG | 6798 |
| 8238 | UGGGCUUU G CAUAUGAC | 2002 | GTCATATG GGCTAGCTACAACGA AAAGCCCA | 6799 |
| 8240 | GGCUUUGC A UAUGACAC | 2003 | GTGTCATA GGCTAGCTACAACGA CCAAAGCC | 6800 |
| 8242 | CUUUGCAU A UGACACCC | 2004 | GGGTGTCA GGCTAGCTACAACGA ATGCAAAG | 6801 |
| 8245 | UGCAUAUG A CACCCGCU | 2005 | AGCGGGTG GGCTAGCTACAACGA CATATGCA | 6802 |
| 8247 | CAUAUGAC A CCCGCUGU | 2006 | ACAGCGGG GGCTAGCTACAACGA GTCATATG | 6803 |
| 8251 | UGACACCC G CUGUUUCG | 2007 | CGAAACAG GGCTAGCTACAACGA GGGTGTCA | 6804 |
| 8254 | CACCCGCU G UUUCGACU | 2008 | AGTCGAU& GGCTAGCTACAACGA AGCGGGTG | 6805 |
| 8260 | CUGUUUCG A CUCAACAG | 2009 | CTGTTGAG GGCTAGCTACAACGA CGAAACAG | 6806 |
| 8265 | UCCACUCA A CAGUCACC | 2010 | GGTGACTG GGCTAGCTACAACGA TGAGTCGA | 6807 |
| 8268 | ACUCAACA G UCACCGAG | 2011 | CTCGGTGA GGCTAGCTACAACGA TGTTGAGT | 6808 |
| 8271 | CAACAGUC A CCGAGAGU | 2012 | ACTCTCGG GGCTAGCTACAACGA GACTGTTG | 6809 |
| 8278 | CACCGAGA G UGACAUCC | 2013 | CGATGTCA GGCTAGCTACAACGA TCTCCGTG | 6810 |
| 8281 | CGAGAGUG A CAUCCGUG | 2014 | CACGGATG GGCTAGCTACAACGA CACTCTCG | 6811 |
| 8283 | AGAGUGAC A UCCGUGUC | 2015 | GACACGGA GGCTAGCTACAACGA GTCACTCT | 6812 |
| 8287 | UGACAUCC G UGUCGAGG | 2016 | CCTCGACA GGCTAGCTACAACGA GGATGTCA | 6813 |
| 8289 | ACAUCCGU G UCGAGGAG | 2017 | CTCCTCGA GGCTAGCTACAACGA ACGGATGT | 6814 |
| 8297 | GUCGAGGA G UCAAUUUA | 2018 | TAAATTGA GGCTAGCTACAACGA TCCTCGAC | 6815 |
| 8301 | AGGAGUCA A UUUACCAA | 2019 | TTGGTAAA GGCTAGCTACAACGA TGACTCCT | 6816 |
| 8305 | GUCAAUUU A CCAAUGUU | 2020 | AACATTGG GGCTAGCTACAACGA AAATTGAC | 6817 |
| 8309 | AUUUACCA A UGUUGUGA | 2021 | TCACAACA GGCTAGCTACAACGA TGGTAAAT | 6818 |
| 8311 | UUACCAAU G UUGUGACU | 2022 | AGTCACAA GGCTAGCTACAACGA ATTGGTAA | 6819 |
| 8314 | CCAAUGUU G UGACUUGG | 2023 | CCAAGTCA GGCTAGCTACAACGA AACATTGG | 6820 |
| 8317 | AUGUUGUG A CUUGGCCC | 2024 | GGGCCAAG GGCTAGCTACAACGA CACAACAT | 6821 |
| 8322 | GUGACUUG G CCCCCGAA | 2025 | TTCGGGGG GGCTAGCTACAACGA CAAGTCAC | 6822 |
| 8331 | CCCCCGAA G CCAGACAG | 2026 | CTGTCTGG GGCTAGCTACAACGA TTCGGGGG | 6823 |
| 8336 | GAAGCCAG A CAGGCCAU | 2027 | ATGGCCTG GGCTAGCTACAACGA CTGGCTTC | 6824 |
| 8340 | CCAGACAG G CCAUAAGG | 2028 | CCTTATCG GGCTAGCTACAACGA CTGTCTGG | 6825 |
| 8343 | GACAGGCC A UAAGGUCG | 2029 | CGACCTTA GGCTAGCTACAACGA GGCCTGTC | 6826 |
| 8348 | GCCAUAAG G UCGCUCAC | 2030 | GTGAGCGA GGCTAGCTACAACGA CTTATGGC | 6827 |
| 8351 | AUAAGGUC G CUCACAGA | 2031 | TCTGTGAG GGCTAGCTACAACGA GACCTTAT | 6828 |
| 8355 | GGUCGCUC A CAGAGCGG | 2032 | CCGCTCTG GGCTAGCTACAACGA GAGCGACC | 6829 |
| 8360 | CUCACAGA G CGGCUUUA | 2033 | TAAAGCCG GGCTAGCTACAACGA TCTGTGAG | 6830 |
| 8363 | ACAGACCG G CUUUAUAU | 2034 | ATATAAAG GGCTAGCTACAACGA CGCTCTGT | 6831 |
| 8368 | GCGGCUUU A UAUCGGGG | 2035 | CCCCGATA GGCTAGCTACAACGA AAAGCCGC | 6832 |
| 8370 | GGCUUUAU A UCGGGGGU | 2036 | ACCCCCGA GGCTAGCTACAACGA ATAAAGCC | 6833 |
| 8377 | UAUCGGGG G UCCUCUGA | 2037 | TCAGACGA GGCTAGCTACAACGA CCCCGATA | 6834 |
| 8385 | GUCCUCUG A CUAAUUCA | 2038 | TGAATTAG GGCTAGCTACAACGA CAGACCAC | 6835 |
| 8389 | UCUGACUA A UUCAAAAG | 2039 | CTTTTGAA GGCTAGCTACAACGA TAGTCAGA | 6836 |
| 8399 | UCAAAAGG G CACAACUG | 2040 | CAGTTCTG GGCTAGCTACAACGA CCTTTTGA | 6837 |
| 8404 | AGGGCAGA A CUGCGGUU | 2041 | AACCGCAG GGCTAGCTACAACGA TCTGCCCT | 6838 |
| 8407 | GCAGAACU G CGGUUAUC | 2042 | GATAACCG GGCTAGCTACAACGA AGTTCTGC | 6839 |
| 8410 | GAACUGCG G UUAUCGCC | 2043 | GGCGATAA GGCTAGCTACAACGA CGCAGTTC | 6840 |
| 8413 | CUGCGGUU A UCGCCGGU | 2044 | ACCGGCGA GGCTAGCTACAACGA AACCGCAG | 6841 |
| 8416 | CGGUUAUC G CCGGUGCC | 2045 | GGCACCGG GGCTAGCTACAACGA GATAACCG | 6842 |
| 8420 | UAUCGCCG G UGCCCCGC | 2046 | GCGCGGCA GGCTAGCTACAACGA CGGCGATA | 6843 |
| 8422 | UCGCCGGU G CCGCGCGA | 2047 | TCGCGCGG GGCTAGCTACAACGA ACCGGCGA | 6844 |
| 8425 | CCCGUGCC G CGCCACCG | 2048 | CGCTCGCG GGCTAGCTACAACGA GGCACCGG | 6845 |
| 8427 | GGUGCCGC G CGAGCGGC | 2049 | GCCGCTCG GGCTAGCTACAACGA GCGGCACC | 6846 |
| 8431 | CCGCGCGA G CGGCGUGC | 2050 | GCACGCCG GGCTAGCTACAACGA TCGCGCGG | 6847 |
| 8434 | CGCGAGCC G CGUGCUGA | 2051 | TCAGCACG GGCTAGCTACAACGA CGCTCCCG | 6848 |
| 8436 | CGAGCCCC G UGCUGACG | 2052 | CGTCAGCA GGCTAGCTACAACGA GCCGCTCG | 6849 |
| 8438 | ACCGGCGU G CUCACCAC | 2053 | CTCCTCAG GGCTAGCTACAACGA ACCCCGCT | 6850 |
| 8442 | GCGUGCUG A CGACCAGC | 2054 | CCTGGTCG GGCTAGCTACAACGA CAGCACGC | 6851 |
| 8445 | UGCUGACG A CCAGCUGU | 2055A | CAGCTGG GGCTAGCTACAACGA CCTCACCA | 6852 |
| 8449 | GACCACGA G CUCUCCUA | 2056 | TACCACAG GGCTAGCTACAACGA TGGTCGTC | 6853 |
| 8452 | CACCACCU G UCGUAAUA | 2057 | TATTACGA GGCTAGCTACAACGA AGCTGGTC | 6854 |
| 8455 | CAGCUGUG G UAAUACCC | 2058 | GGGTATTA GGCTAGCTACAACGA CACAGCTC | 6855 |
| 8458 | CUGUGGUA A UACCCUCA | 2059 | TGAGGGTA GGCTAGCTACAACGA TACCACAC | 6856 |
| 8460 | GUGGUAAU A CCCUCACA | 2060 | TGTGAGGC GGCTAGCTACAACGA ATTACCAC | 6857 |
| 8466 | AUACCCUC A CAUGUUAC | 2061 | GTAACATG GGCTAGCTACAACGA GAGGGTAT | 6858 |
| 8468 | ACCCUCAC A UGUUACUU | 2062 | AAGTAACA GGCTAGCTACAACGA GTGAGGGT | 6859 |
| 8470 | CCUCACAU G UUACUUGA | 2063 | TCAAGTAA GGCTAGCTACAACGA ATGTGACG | 6860 |
| 8473 | CACAUGUU A CUUGAAAG | 2064 | CTTTCAAC GGCTAGCTACAACGA AACATGTG | 6861 |
| 8481 | ACUUGAAA G CCUCUGCG | 2065 | CGCAGAGG GGCTAGCTACAACGA TTTCAACT | 6862 |
| 8487 | AAGCCUCU G CGGCCUGU | 2066 | ACAGGCCG GGCTAGCTACAACGA AGAGGCTT | 6863 |
| 8490 | CCUCUGCG G CCUGUCGA | 2067 | TCGACAGG GGCTAGCTACAACGA CGCACAGC | 6864 |
| 8494 | UGCCGCCU G UCGAGCUG | 2068 | CAGCTCGA GGCTAGCTACAACGA AGGCCGCA | 6865 |
| 8499 | CCUGUCGA G CUGCGAAG | 2069 | CTTCGCAG GGCTAGCTACAACGA TCGACAGG | 6866 |
| 8502 | CUCCACCU G CCAACCUC | 2070 | CACCTTCC GGCTAGCTACAACGA ACCTCCAC | 6867 |
| 8507 | CCUGCGAA G CUCCAGGA | 2071 | TCCTGGAG GGCTAGCTACAACGA TTCCCAGC | 6868 |
| 8515 | GCUCCAGG A CUGCACGA | 2072 | TCGTGCAC GGCTAGCTACAACGA CCTCGAGC | 6869 |
| 8518 | CCACGACU G CACGAUGC | 2073 | GCATCGTC GGCTAGCTACAACGA AGTCCTGG | 6870 |
| 8520 | AGGACUGC A CGAUGCUC | 2074 | GAGCATCG GGCTAGCTACAACGA GCAGTCCT | 6871 |
| 8523 | ACUCCACG A UGCUCCUC | 2075 | CACGACGA GGCTAGCTACAACGA CCTCCACT | 6872 |
| 8525 | UCCACCAU G CUCCUGUG | 2076 | CACACCAC GGCTAGCTACAACGA ATCCTGCA | 6873 |
| 8529 | CCAUCCUC G UCUCUGGA | 2077 | TCCACACA GGCTAGCTACAACGA GAGCATCG | 6874 |
| 8531 | AUGCUCCU G UGUCCAGA | 2078 | TCTCCACA GGCTAGCTACAACGA ACCACCAT | 6875 |
| 8533 | GCUCCUGU G UGGAGACG | 2079 | CGTCTCGA GGCTAGCTACAACGA ACACGAGC | 6876 |
| 8539 | CUGUCCAC A CCACCUGC | 2080 | CCAGCTCC GGCTAGCTACAACGA CTCCACAC | 6877 |
| 8542 | UGGAGACC A CCUGGUCC | 2081 | CGACCAGG GGCTAGCTACAACGA CGTCTCCA | 6878 |
| 8547 | ACCACCUC G UCCUUAUC | 2082 | CATAACGA GGCTAGCTACAACGA CACGTCGT | 6879 |
| 8550 | ACCUGGUC G UUAUCUGU | 2083 | ACAGATAA GGCTAGCTACAACGA GACCACCT | 6880 |
| 8553 | UGGUCCUC A UCUGUGAA | 2084 | TTCACAGA GGCTAGCTACAACGA AACCACCA | 6881 |
| 8557 | CCUUAUCU G UGAAACUC | 2085 | CACTTTCA GGCTAGCTACAACGA AGATAACG | 6882 |
| 8563 | CUCUGAAA G UGCGGCGA | 2086 | TCCCCGCA GGCTAGCTACAACGA TTTCACAG | 6883 |
| 8565 | CUGAAACU G CCGCGACC | 2087 | CCTCCCCG GGCTAGCTACAACGA ACTTTCAC | 6884 |
| 8571 | CUCCCCCC A CCCAACAC | 2088 | CTCTTCCG GGCTAGCTACAACGA CCCCGCAC | 6885 |
| 8581 | CCAAGAGG A CGCGGCGA | 2089 | TCGCCGCG GGCTAGCTACAACGA CCTCTTGG | 6886 |
| 8583 | AAGAGGAC G CCCCGAGC | 2090 | CCTCCCCC GGCTAGCTACAACGA GTCCTCTT | 6887 |
| 8586 | ACGACGCG G CCAGCCUA | 2091 | TACGCTCG GGCTAGCTACAACGA CCCCTCCT | 6888 |
| 8590 | CGCGGCGA G CCUACGAG | 2092 | CTCGTACG GGCTAGCTACAACGA TCGCCGCG | 6889 |
| 8594 | CCCACCCU A CGACUCUU | 2093 | AACACTCC GGCTAGCTACAACGA ACCCTCCC | 6890 |
| 8598 | CCCUACGA G UCUUCACC | 2094 | CCTCAACA GGCTAGCTACAACGA TCCTACCC | 6891 |
| 8604 | GAGUCUUC A CCGACGCU | 2095 | AGCCTCCC GGCTAGCTACAACGA GAACACTC | 6892 |
| 8610 | UCACGGAG G CUAUGACU | 2096 | AGTCATAG GGCTAGCTACAACGA CTCCGTGA | 6893 |
| 8613 | CCCACGCU A UCACUAGG | 2097 | CCTACTCA GGCTAGCTACAACGA AGCCTCCC | 6894 |
| 8616 | ACGCUAUG A CUAGGUAC | 2098 | GTACCTAG GGCTAGCTACAACGA CATAGCCT | 6895 |
| 8621 | AUCACUAC G UACUCUCC | 2099 | CCACACTA GGCTAGCTACAACGA CTACTCAT | 6896 |
| 8623 | GACUAGGU A CUCUGCCC | 2100 | GGGCAGAG GGCTAGCTACAACGA ACCTAGTC | 6897 |
| 8628 | GGUACUCU G CCCCCCCC | 2101 | GGGGGGGG GGCTAGCTACAACGA AGACTACC | 6898 |
| 8641 | CCCCCCGC A CCCCCCCC | 2102 | GGCGCGGG GGCTAGCTACAACGA CCCCGGGG | 6899 |
| 8645 | CCCCACCC G CCCCAACC | 2103 | CCTTGGGC GGCTAGCTACAACGA GGCTCCCC | 6900 |
| 8651 | CCGCCCCA A CCGGAAUA | 2104 | TATTCCGG GGCTAGCTACAACGA TGGGGCGG | 6901 |
| 8657 | CAACCGGA A UACGACUU | 2105 | AAGTCGTA GGCTAGCTACAACGA TCCGGTTG | 6902 |
| 8659 | ACCGGAAU A CGACUUGG | 2106 | CCAAGTCG GGCTAGCTACAACGA ATTCCGGT | 6903 |
| 8662 | GGAAUACG A CUUGGAGU | 2107 | ACTCCAAG GGCTAGCTACAACGA CGTATTCC | 6904 |
| 8669 | GACUUGGA G UUGAUAAC | 2108 | GTTATCAA GGCTAGCTACAACGA TCCAAGTC | 6905 |
| 8673 | UGGAGUUG A UAACAUCA | 2109 | TGATGTTA GGCTAGCTACAACGA CAACTCCA | 6906 |
| 8676 | AGUUGAUA A CAUCAUGC | 2110 | GCATGATG GGCTAGCTACAACGA TATCAACT | 6907 |
| 8678 | UUGAUAAC A UCAUGCUC | 2111 | GAGCATGA GGCTAGCTACAACGA GTTATCAA | 6908 |
| 8681 | AUAACAUC A UGCUCCUC | 2112 | GAGGAGCA GGCTAGCTACAACGA GATGTTAT | 6909 |
| 8683 | AACAUCAU G CUCCUCGA | 2113 | TGGAGGAG GGCTAGCTACAACGA ATGATGTT | 6910 |
| 8692 | CUCCUCCA A CGUAUCAG | 2114 | CTGATACG GGCTAGCTACAACGA TGGAGGAG | 6911 |
| 8694 | CCUCCAAC G UAUCAGUU | 2115 | AACTGATA GGCTAGCTACAACGA GTTGGAGG | 6912 |
| 8696 | UCCAACGU A UCAGUUGC | 2116 | GCAACTGA GGCTAGCTACAACGA ACGTTGGA | 6913 |
| 8700 | ACCUAUCA G UUGCACAC | 2117 | GTGTGCAA GGCTAGCTACAACGA TGATACGT | 6914 |
| 8703 | UAUCAGUU G CACACGAU | 2118 | ATCGTGTG GGCTAGCTACAACGA AACTGATA | 6915 |
| 8705 | UCAGUUGC A CACGAUGC | 2119 | GCATCGTG GGCTAGCTACAACGA GCAACTGA | 6916 |
| 8707 | AGUUGCAC A CGAUGCAU | 2120 | ATGCATCG GGCTAGCTACAACGA GTGCAACT | 6917 |
| 8710 | UGCACACG A UGCAUCUG | 2121 | CAGATGCA GGCTAGCTACAACGA CGTGTGCA | 6918 |
| 8712 | CACACGAU G CAUCUGGC | 2122 | GCCAGATG GGCTAGCTACAACGA ATCGTGTG | 6919 |
| 8714 | CACGAUGC A UCUGGCAA | 2123 | TTGCCAGA GGCTAGCTACAACGA GCATCGTG | 6920 |
| 8719 | UCCAUCUG G CAAAAGGG | 2124 | CCCTTTTG GGCTAGCTACAACGA CAGATGCA | 6921 |
| 8727 | GCAAAAGG G UGUACUAC | 2125 | GTAGTACA GGCTAGCTACAACGA CCTTTTGC | 6922 |
| 8729 | AAAAGGGU G UACUACCU | 2126 | AGGTAGTA GGCTAGCTACAACGA ACCCTTTT | 6923 |
| 8731 | AAGGGUGU A CUACCUCA | 2127 | TGAGGTAG GGCTAGCTACAACGA ACACCCTT | 6924 |
| 8734 | GGUGUACU A CCUCACCC | 2128 | GGGTGAGG GGCTAGCTACAACGA AGTACACC | 6925 |
| 8739 | ACUACCUC A CCCGUGAC | 2129 | GTCACGGG GGCTAGCTACAACGA GAGGTAGT | 6926 |
| 8743 | CCUCACCC G UGACCCGA | 2130 | TGGGGTCA GGCTAGCTACAACGA GGGTGAGG | 6927 |
| 8746 | CACCCGUG A CCCCACGA | 2131 | TGGTGGGG GGCTAGCTACAACGA CACGGGTG | 6928 |
| 8751 | GUGACCCC A CCACCCCC | 2132 | GGGGGTGG GGCTAGCTACAACGA GGGGTCAC | 6929 |
| 8754 | ACCCCACC A CCCCCCUU | 2133 | AAGGGGGG GGCTAGCTACAACGA GGTGGGGT | 6930 |
| 8763 | CCCCCCUU G CGCGGGCU | 2134 | AGCCCGCG GGCTAGCTACAACGA AAGGGGGG | 6931 |
| 8765 | CCCCUUGC G CGGGCUGC | 2135 | GCAGCCCG GGCTAGCTACAACGA GCAAGGGG | 6932 |
| 8769 | UUGCGCGG G CUGCGUGG | 2136 | CCACGCAG GGCTAGCTACAACGA CCGCGCAA | 6933 |
| 8772 | CGCGGGCU G CGUGGGAG | 2137 | CTCCCACG GGCTAGCTACAACGA AGCCCGCG | 6934 |
| 8774 | CGGGCUGC G UGGGAGAC | 2138 | GTCTCCGA GGCTAGCTACAACGA GCAGCCCG | 6935 |
| 8781 | CGUGGGAG A CAGCUAGA | 2139 | TCTAGCTG GGCTAGCTACAACGA CTCCCACG | 6936 |
| 8784 | GGGAGACA G CUAGAAGC | 2140 | GCTTCTAG GGCTAGCTACAACGA TGTCTCCC | 6937 |
| 8791 | AGCUAGAA G CACUCCAG | 2141 | CTGGAGTG GGCTAGCTACAACGA TTCTAGCT | 6938 |
| 8793 | CUAGAAGC A CUCCAGUC | 2142 | GACTGGAG GGCTAGCTACAACGA GCTTCTAG | 6939 |
| 8799 | GCACUCGA G UCAACUCC | 2143 | GGAGTTGA GGCTAGCTACAACGA TGGAGTGC | 6940 |
| 8803 | UCCAGUCA A CUCCUGGC | 2144 | GCCAGGAG GGCTAGCTACAACGA TGACTGGA | 6941 |
| 8810 | AACUCCUG G CUAGGCAA | 2145 | TTGCCTAG GGCTAGCTACAACGA CAGGAGTT | 6942 |
| 8815 | CUGGCUAG G CAACAUCA | 2146 | TGATGTTG GGCTAGCTACAACGA CTAGCCAG | 6943 |
| 8818 | GCUAGGCA A CAUCAUCA | 2147 | TGATGATG GGCTAGCTACAACGA TGGCTAGC | 6944 |
| 8820 | UAGGCAAC A UCAUCAUG | 2148 | CATGATGA GGCTAGCTACAACGA GTTGCCTA | 6945 |
| 8823 | GCAACAUC A UCAUGUUU | 2149 | AAACATGA GGCTAGCTACAACGA GATGTTGC | 6946 |
| 8826 | ACAUCAUC A UGUUUGCA | 2150 | TGCAAACA GGCTAGCTACAACGA GATGATGT | 6947 |
| 8828 | AUCAUCAU G UUUGCACC | 2151 | GGTGCAAA GGCTAGCTACAACGA ATGATGAT | 6948 |
| 8832 | UCAUGUUU G CACCCACU | 2152 | AGTGGGTG GGCTAGCTACAACGA AAACATGA | 6949 |
| 8834 | AUGUUUGC A CCCACUCU | 2153 | AGAGTGGG GGCTAGCTACAACGA GCAAACAT | 6950 |
| 8838 | UUGCACCC A CUCUAUGG | 2154 | CCATAGAG GGCTAGCTACAACGA GGGTGCAA | 6951 |
| 8843 | CCCACUCU A UGGGUAAG | 2155 | CTTACCGA GGCTAGCTACAACGA AGAGTGGG | 6952 |
| 8847 | CUCUAUGG G UAAGGAUG | 2156 | CATCCTTA GGCTAGCTACAACGA CCATAGAG | 6953 |
| 8853 | GGGUAAGG A UGAUUCUG | 2157 | CAGAATCA GGCTAGCTACAACGA CCTTACCC | 6954 |
| 8856 | UAAGGAUG A UUCUGAUG | 2158 | CATCAGAA GGCTAGCTACAACGA CATCCTTA | 6955 |
| 8862 | UGAUUCUG A UGACUCAC | 2159 | GTGAGTCA GGCTAGCTACAACGA CAGAATCA | 6956 |
| 8865 | UUCUGAUG A CUCACUUC | 2160 | GAAGTGAG GGCTAGCTACAACGA CATCAGAA | 6957 |
| 8869 | GAUGACUC A CUUCUUCU | 2161 | AGAAGAAG GGCTAGCTACAACGA GAGTCATC | 6958 |
| 8880 | UCUUCUCC A UCCUUCUA | 2162 | TAGAAGGA GGCTAGCTACAACGA GGAGAAGA | 6959 |
| 8889 | UCCUUCUA G CCCAGGAG | 2163 | CTCCTGGG GGCTAGCTACAACGA TAGAAGGA | 6960 |
| 8897 | GCCCAGGA G CAACUUGA | 2164 | TCAAGTTG GGCTAGCTACAACGA TCCTGGGC | 6961 |
| 8900 | CAGGAGCA A CUUGAGAA | 2165 | TTCTCAAG GGCTAGCTACAACGA TGCTCCTG | 6962 |
| 8910 | UUGAGAAA G CCCUAGAC | 2166 | GTCTAGGG GGCTAGCTACAACGA TTTCTCAA | 6963 |
| 8917 | AGCCCUAG A CUGCCAGA | 2167 | TCTCGCAG GGCTAGCTACAACGA CTAGGGCT | 6964 |
| 8920 | CCUAGACU G CCAGAUCU | 2168 | AGATCTGG GGCTAGCTACAACGA AGTCTAGG | 6965 |
| 8925 | ACUGCCAG A UCUACGGG | 2169 | CCCGTAGA GGCTAGCTACAACGA CTGGCAGT | 6966 |
| 8929 | CCAGAUCU A CGGGGCUU | 2170 | AAGCCCCG GGCTAGCTACAACGA AGATCTGG | 6967 |
| 8934 | UCUACGGG G CUUGUUAC | 2171 | GTAACAAG GGCTAGCTACAACGA CCCGTAGA | 6968 |
| 8938 | CGGGGCUU G UUACUCGA | 2172 | TGGAGTAA GGCTAGCTACAACGA AAGCCCCG | 6969 |
| 8941 | GGCUUGUU A CUCCAUUG | 2173 | CAATGGAG GGCTAGCTACAACGA AACAAGCC | 6970 |
| 8946 | GUUACUCC A UUGAGCGA | 2174 | TGGCTCAA GGCTAGCTACAACGA GGAGTAAC | 6971 |
| 8951 | UCCAUUGA G CCACUUGA | 2175 | TCAAGTGG GGCTAGCTACAACGA TCAATGGA | 6972 |
| 8954 | AUUGAGCC A CUUGACCU | 2176 | AGGTCAAG GGCTAGCTACAACGA GGCTCAAT | 6973 |
| 8959 | GCCACUUG A CCUACCUC | 2177 | GAGGTAGG GGCTAGCTACAACGA CAAGTGGC | 6974 |
| 8963 | CUUGACCU A CCUCAGAU | 2178 | ATCTGAGG GGCTAGCTACAACGA AGGTCAAG | 6975 |
| 8970 | UACCUCAG A UCAUUCAG | 2179 | CTGAATGA GGCTAGCTACAACGA CTGAGGTA | 6976 |
| 8973 | CUCAGAUC A UUCAGCGA | 2180 | TCGCTGAA GGCTAGCTACAACGA GATCTGAG | 6977 |
| 8978 | AUCAUUCA G CGACUCCA | 2181 | TGGAGTCG GGCTAGCTACAACGA TGAATGAT | 6978 |
| 8981 | AUUCAGCG A CUCCAUGG | 2182 | CCATGGAG GGCTAGCTACAACGA CGCTGAAT | 6979 |
| 8986 | GCGACUCC A UGGUCUUA | 2183 | TAAGACGA GGCTAGCTACAACGA GGAGTCGC | 6980 |
| 8989 | ACUCCAUG G UCUUAGCG | 2184 | CGCTAAGA GGCTAGCTACAACGA CATGGAGT | 6981 |
| 8995 | UGGUCUUA G CGCAUUUU | 2185 | AAAATGCG GGCTAGCTACAACGA TAAGACCA | 6982 |
| 8997 | CUCUUAGC G CAUUUUCA | 2186 | TGAAAATG GGCTAGCTACAACGA GCTAAGAC | 6983 |
| 8999 | CUUAGCGC A UUUUCACU | 2187 | AGTGAAAA GGCTAGCTACAACGA GCGCTAAG | 6984 |
| 9005 | GCAUUUUC A CUCCAUAG | 2188 | CTATGGAG GGCTAGCTACAACGA GAAAATGC | 6985 |
| 9010 | UUCACUCC A UAGUUACU | 2189 | AGTAACTA GGCTAGCTACAACGA GGAGTGAA | 6986 |
| 9013 | ACUCCAUA G UUACUCCC | 2190 | GGGAGTAA GGCTAGCTACAACGA TATGGAGT | 6987 |
| 9016 | CCAUAGUU A CUCCCCAG | 2191 | CTGGGGAG GGCTAGCTACAACGA AACTATGG | 6988 |
| 9025 | CUCCCCAG G UGAAAUCA | 2192 | TGATTTCA GGCTAGCTACAACGA CTGGGGAG | 6989 |
| 9030 | CAGGUGAA A UCAAUAGG | 2193 | CCTATTGA GGCTAGCTACAACGA TTCACCTG | 6990 |
| 9034 | UGAAAUCA A UAGGGUGG | 2194 | CCACCCTA GGCTAGCTACAACGA TGATTTCA | 6991 |
| 9039 | UCAAUAGG G UGGCAUCA | 2195 | TGATGCGA GGCTAGCTACAACGA CCTATTGA | 6992 |
| 9042 | AUAGGGUG G CAUCAUGC | 2196 | GCATGATG GGCTAGCTACAACGA CACCCTAT | 6993 |
| 9044 | AGGGUGGC A UCAUGCCU | 2197 | AGGCATGA GGCTAGCTACAACGA GCCACCCT | 6994 |
| 9047 | GUGGCAUC A UGCCUCAG | 2198 | CTGAGGCA GGCTAGCTACAACGA GATGCCAC | 6995 |
| 9049 | GGCAUCAU G CCUCAGGA | 2199 | TCCTGAGG GGCTAGCTACAACGA ATGATGCC | 6996 |
| 9059 | CUCAGGAA A CUUGGGGU | 2200 | ACCCCAAG GGCTAGCTACAACGA TTCCTGAG | 6997 |
| 9066 | AACUUGGG G UACCACCC | 2201 | GGGTGGTA GGCTAGCTACAACGA CCCAAGTT | 6998 |
| 9068 | CUUGGGGU A CCACCCUU | 2202 | AAGGGTGG GGCTAGCTACAACGA ACCCCAAG | 6999 |
| 9071 | GGGGUACC A CCCUUGCG | 2203 | CGCAAGGG GGCTAGCTACAACGA GGTACCCC | 7000 |
| 9077 | CCACCCUU G CGAACCUG | 2204 | CAGGTTCG GGCTAGCTACAACGA AAGGGTGG | 7001 |
| 9081 | CCUUGCCA A CCUGGAGA | 2205 | TCTCCAGG GGCTAGCTACAACGA TCGCAAGG | 7002 |
| 9089 | ACCUGGAG A CAUCGGGC | 2206 | GCCCGATG GGCTAGCTACAACGA CTCCAGGT | 7003 |
| 9091 | CUGGAGAC A UCGGGCCA | 2207 | TGGCCCGA GGCTAGCTACAACGA GTCTCCAG | 7004 |
| 9096 | GACAUCGG G CCAGAAGU | 2208 | ACTTCTGG GGCTAGCTACAACGA CCGATGTC | 7005 |
| 9103 | GGCCAGAA G UGUUCCCC | 2209 | CGCCAACA GGCTAGCTACAACGA TTCTCCCC | 7006 |
| 9105 | CCAGAACU G UUCGCGCU | 2210 | AGCGCGAA GGCTAGCTACAACGA ACTTCTGG | 7007 |
| 9109 | AACUGUUC G CGCUAAGC | 2211 | CCTTACCG GGCTAGCTACAACGA CAACACTT | 7008 |
| 9111 | GUGUUCGC G CUAAGCUA | 2212 | TAGCTTAG GGCTAGCTACAACGA GCCAACAC | 7009 |
| 9116 | CCCCCUAA G CUACUGUC | 2213 | CACACTAC GGCTAGCTACAACGA TTACCCCG | 7010 |
| 9119 | GCUAAGCU A CUGUCCCA | 2214 | TGGGACAG GGCTAGCTACAACGA AGCTTAGC | 7011 |
| 9122 | AAGCUACU G UCCCACCG | 2215 | CCCTGCGA GGCTAGCTACAACGA AGTAGCTT | 7012 |
| 9138 | GCGGGAGG G CCGCCACC | 2216 | GGTGGCGG GGCTAGCTACAACGA CCTCCCCC | 7013 |
| 9141 | CCAGGGCC G CCACCUGU | 2217 | ACAGCTGG GGCTAGCTACAACGA GGCCCTCC | 7014 |
| 9144 | GGGCCGCC A CCUGUGGC | 2218 | GCCACAGG GGCTAGCTACAACGA GGCGGCCC | 7015 |
| 9148 | CGCCACCU G UGGCAGGU | 2219 | ACCTGCGA GGCTAGCTACAACGA ACCTCGCG | 7016 |
| 9151 | CACCUGUG G CAGGUACC | 2220 | GGTACCTG GGCTAGCTACAACGA CACAGGTG | 7017 |
| 9155 | UGUGGCAG G UACCUCUU | 2221 | AAGAGGTA GGCTAGCTACAACGA CTGCCACA | 7018 |
| 9157 | UGGCAGGU A CCUCUUCA | 2222 | TGAAGAGG GGCTAGCTACAACGA ACCTGCCA | 7019 |
| 9166 | CCUCUUCA A CUGGGCAG | 2223 | CTGCCCAG GGCTAGCTACAACGA TGAAGAGG | 7020 |
| 9171 | UCAACUGG G CAGUAAAG | 2224 | CTTTACTG GGCTAGCTACAACGA CCAGTTGA | 7021 |
| 9174 | ACUGGGCA G UAAAGACC | 2225 | GGTCTTTA GGCTAGCTACAACGA TGCCCAGT | 7022 |
| 9180 | CAGUAAAG A CCAAACUC | 2226 | GAGTTTGG GGCTAGCTACAACGA CTTTACTG | 7023 |
| 9185 | AAGACCAA A CUCAAACU | 2227 | AGTTTGAG GGCTAGCTACAACGA TTGGTCTT | 7024 |
| 9191 | AAACUCAA A CUCACUCC | 2228 | GGAGTGAG GGCTAGCTACAACGA TTGAGTTT | 7025 |
| 9195 | UCAAACUC A CUCCAAUC | 2229 | GATTGGAG GGCTAGCTACAACGA GAGTTTGA | 7026 |
| 9201 | UCACUCCA A UCCCAGCU | 2230 | AGCTGGGA GGCTAGCTACAACGA TGGAGTGA | 7027 |
| 9207 | CAAUCCGA G CUGCGUCU | 2231 | AGACGCAG GGCTAGCTACAACGA TGGGATTG | 7028 |
| 9210 | UCCCAGCU G CGUCUCAG | 2232 | CTGAGACG GGCTAGCTACAACGA AGCTGGGA | 7029 |
| 9212 | CCAGCUGC G UCUCAGUU | 2233 | AACTGAGA GGCTAGCTACAACGA GCAGCTGG | 7030 |
| 9218 | GCGUCUCA G UUGGACUU | 2234 | AAGTCCAA GGCTAGCTACAACGA TGAGACGC | 7031 |
| 9223 | UCAGUUGG A CUUGUCCA | 2235 | TGGACAAG GGCTAGCTACAACGA CCAACTGA | 7032 |
| 9227 | UUGGACUU G UCCAACUG | 2236 | CAGTTGGA GGCTAGCTACAACGA AAGTCCAA | 7033 |
| 9232 | CUUGUCCA A CUGGUUCG | 2237 | CGAACCAG GGCTAGCTACAACGA TGGACAAG | 7034 |
| 9236 | UCCAACUG G UUCGUUGC | 2238 | GCAACGAA GGCTAGCTACAACGA CAGTTGGA | 7035 |
| 9240 | ACUGGUUC G UUGCUGGC | 2239 | GCCAGCAA GGCTAGCTACAACGA GAACCAGT | 7036 |
| 9243 | GGUUCGUU G CUGGCUAC | 2240 | GTAGCCAG GGCTAGCTACAACGA AACGAACC | 7037 |
| 9247 | CGUUGCUG G CUACAGCG | 2241 | CGCTGTAG GGCTAGCTACAACGA CAGCAACG | 7038 |
| 9250 | UGCUGGCU A CAGCGGGG | 2242 | CCCCGCTG GGCTAGCTACAACGA AGCCAGCA | 7039 |
| 9253 | UGGCUACA G CGGGGGAG | 2243 | CTCCCCCG GGCTAGCTACAACGA TGTAGCCA | 7040 |
| 9262 | CGGGGGAG A CGUGUAUC | 2244 | GATACACG GGCTAGCTACAACGA CTCCCCCG | 7041 |
| 9264 | GGGGAGAC G UGUAUCAC | 2245 | GTGATACA GGCTAGCTACAACGA GTCTCCCC | 7042 |
| 9266 | GGAGACGU G UAUCACAG | 2246 | CTGTGATA GGCTAGCTACAACGA ACGTCTCC | 7043 |
| 9268 | AGACGUGU A UCACAGCC | 2247 | GGCTGTGA GGCTAGCTACAACGA ACACGTCT | 7044 |
| 9271 | CGUGUAUC A CAGCCUGU | 2248 | ACAGGCTG GGCTAGCTACAACGA GATACACG | 7045 |
| 9274 | GUAUCACA G CCUGUCUC | 2249 | GAGACAGG GGCTAGCTACAACGA TGTGATAC | 7046 |
| 9278 | CACAGCCU G UCUCGUGC | 2250 | GCACGAGA GGCTAGCTACAACGA AGGCTGTG | 7047 |
| 9283 | CCUGUCUC G UGCCCGAC | 2251 | GTCGGGCA GGCTAGCTACAACGA GAGACAGG | 7048 |
| 9285 | UGUCUCGU G CCCGACCC | 2252 | GGGTCGGG GGCTAGCTACAACGA ACGAGACA | 7049 |
| 9290 | CGUGCCCG A CCCCGCUG | 2253 | CAGCGGGG GGCTAGCTACAACGA CGGGCACG | 7050 |
| 9295 | CCGACCCC G CUGGUUCA | 2254 | TGAACCAG GGCTAGCTACAACGA GGGGTCGG | 7051 |
| 9299 | CCCCGCUG G UUCAUGCU | 2255 | AGCATGAA GGCTAGCTACAACGA CAGCGGGG | 7052 |
| 9303 | GCUGGUUC A UGCUUUGC | 2256 | GCAAAGCA GGCTAGCTACAACGA GAACCAGC | 7053 |
| 9305 | UGGUUCAU G CUUUGCCU | 2257 | AGGCAAAG GGCTAGCTACAACGA ATGAACCA | 7054 |
| 9310 | CAUGCUUU G CCUACUCC | 2258 | GGAGTAGG GGCTAGCTACAACGA AAAGCATG | 7055 |
| 9314 | CUUUGCCU A CUCCUACU | 2259 | AGTAGGAG GGCTAGCTACAACGA AGGCAAAG | 7056 |
| 9320 | CUACUCCU A CUCUCCGU | 2260 | ACGGAGAG GGCTAGCTACAACGA AGGAGTAG | 7057 |
| 9327 | UACUCUCC G UAGGGGUA | 2261 | TACCCCTA GGCTAGCTACAACGA GGAGAGTA | 7058 |
| 9333 | CCGUAGGG G UAGGCAUC | 2262 | GATGCCTA GGCTAGCTACAACGA CCCTACGG | 7059 |
| 9337 | AGGGGUAG G CAUCUACC | 2263 | GGTAGATG GGCTAGCTACAACGA CTACCCCT | 7060 |
| 9339 | GGGUAGGC A UCUACCUG | 2264 | CAGGTAGA GGCTAGCTACAACGA GGCTACCC | 7061 |
| 9343 | AGGCAUCU A CCUGCUCC | 2265 | GGAGCAGG GGCTAGCTACAACGA AGATGCCT | 7062 |
| 9347 | AUCUACCU G CUCCCCAA | 2266 | TTGGGGAG GGCTAGCTACAACGA AGGTAGAT | 7063 |
| 9355 | GCUCCCCA A CCGAUGAA | 2267 | TTCATCGG GGCTAGCTACAACGA TGGGGAGC | 7064 |
| 9359 | CCCAACCG A UGAACAGG | 2268 | CCTGTTCA GGCTAGCTACAACGA CGGTTGGG | 7065 |
| 9363 | ACCGAUGA A CAGGGAGC | 2269 | GCTCCCTG GGCTAGCTACAACGA TCATCGGT | 7066 |
| 9370 | AACAGGGA G CUAAACAC | 2270 | GTGTTTAG GGCTAGCTACAACGA TCCCTGTT | 7067 |
| 9375 | GGAGCUAA A CACUCCAG | 2271 | CTGGAGTG GGCTAGCTACAACGA TTAGCTCC | 7068 |
| 9377 | AGCUAAAC A CUCCAGGC | 2272 | GCCTGGAG GGCTAGCTACAACGA GTTTAGCT | 7069 |
| 9384 | CACUCCAG G CCAAUAGG | 2273 | CCTATTGG GGCTAGCTACAACGA CTGGAGTG | 7070 |
| 9388 | CCAGGCCA A UAGGCCAU | 2274 | ATGGCCTA GGCTAGCTACAACGA TGGCCTGG | 7071 |
| 9392 | GCCAAUAG G CCAUCCCG | 2275 | CGGGATGG GGCTAGCTACAACGA CTATTGGC | 7072 |
| 9395 | AAUAGGCC A UCCCGUUU | 2276 | AAACGGGA GGCTAGCTACAACGA GGCCTATT | 7073 |
| 9400 | GCCAUCCC G UUUUUUUU | 2277 | AAAAAAAA GGCTAGCTACAACGA GGGATGGC | 7074 |

Input Sequence = HPCK1S1. Cut Site = R/Y
Arm Length = 8. Core Sequence=GGCTAGCTACAACGA
HPCK1S1 Hepatitis G virus (strain HCV-1b, Clone HCV-K1-S1), Complete genome; acc#
gi|1030702|dbj|D50483.1; 9410 nt

TABLE IV

HCV minus strand DNAzyme and Substrate Sequence

| | | | | |
|---|---|---|---|---|
| Pos | Substrate | Seq ID | DNAzyzne | Seq ID |

| | | | | |
|---|---|---|---|---|
| 9413 | AAAAAAAA A CGGGAUGG | 2278 | CCATCCCG GGCTAGCTACAACGA TTTTTTTT | 7075 |
| 9408 | AAAACGGG A UGGCCUAU | 2279 | ATAGGCCA GGCTAGCTACAACGA CCCGTTTT | 7076 |
| 9405 | ACGGGAUG G CCUAUUGG | 2280 | CCAATAGG GGCTAGCTACAACGA CATCCCGT | 7077 |
| 9401 | GAUGGCCU A UUGGCCUG | 2281 | CAGGCCAA GGCTAGCTACAACGA AGGCCATC | 7078 |
| 9397 | GCCUAUUG G CCUGGAGU | 2282 | ACTCCAGG GGCTAGCTACAACGA CAATAGGC | 7079 |
| 9390 | GGCCUGGA G UGUUUAGC | 2283 | GCTAAACA GGCTAGCTACAACGA TCCAGGCC | 7080 |
| 9388 | CCUGGAGU G UUUAGCUC | 2284 | GAGCTAAA GGCTAGCTACAACGA ACTCCAGG | 7081 |
| 9383 | AGUGUUUA G CUCCCUGU | 2285 | ACAGGGAG GGCTAGCTACAACGA TAAACACT | 7082 |
| 9376 | AGCUCCCU G UUCAUCGG | 2286 | CCAACCGA GGCTAGCTACAACGA AGGGAGCT | 7083 |
| 9372 | CCCUGUUC A UCGGUUGG | 2287 | CCAACCGA GGCTAGCTACAACGA GAACAGGG | 7084 |
| 9368 | GUUCAUCG G UUGGGGAG | 2288 | CTCCCCAA GGCTAGCTACAACGA CGATGAAC | 7085 |
| 9360 | GUUGGGGA G CAGGUAGA | 2289 | TCTACCTG GGCTAGCTACAACGA TCCCCAAC | 7086 |
| 9356 | GGGAGCAG G UAGAUGCC | 2290 | GGCATCTA GGCTAGCTACAACGA CTGCTCCC | 7087 |
| 9352 | GCAGGUAG A UGCCUACC | 2291 | GGTAGGCA GGCTAGCTACAACGA CTACCTGC | 7088 |
| 9350 | AGGUAGAU G CCUACCCC | 2292 | GGGGTAGG GGCTAGCTACAACGA ATCTACCT | 7089 |
| 9346 | AGAUGCCU A CCCCAUCG | 2293 | CGTAGGGG GGCTAGCTACAACGA AGGCATCT | 7090 |
| 9340 | CUACCCCU A CGGAGAGU | 2294 | ACTCTCCG GGCTAGCTACAACGA AGGGGTAG | 7091 |
| 9333 | UACGGAGA G UAGGAGUA | 2295 | TACTCCTA GGCTAGCTACAACGA TCTCCGTA | 7092 |
| 9327 | GAGUAGGA G UAGGCAAA | 2296 | TTTGCCTA GGCTAGCTACAACGA TCCTACTC | 7093 |
| 9323 | AGGAGUAG G CAAAGCAU | 2297 | ATGCTTTG GGCTAGCTACAACGA CTACTCCT | 7094 |
| 9318 | UAGGCAAA G CAUGAACC | 2298 | GGTTCATG GGCTAGCTACAACGA TTTGCCTA | 7095 |
| 9316 | GGCAAAGC A UGAACCAG | 2299 | CTGGTTCA GGCTAGCTACAACGA GCTTTGCC | 7096 |
| 9312 | AAGCAUGA A CCAGCGGG | 2300 | CCCGCTGG GGCTAGCTACAACGA TCATGCTT | 7097 |
| 9308 | AUGAACCA G CGGGGUCG | 2301 | CGACCCCG GGCTAGCTACAACGA TGGTTCAT | 7098 |
| 9303 | CCAGCGGG G UCGGGCAC | 2302 | GTGCCCGA GGCTAGCTACAACGA CCCGCTGG | 7099 |
| 9298 | GGGGUCGG G CACGAGAC | 2303 | GTCTCGTG GGCTAGCTACAACGA CCGACCCC | 7100 |
| 9296 | GGUCGGGC A CGAGACAG | 2304 | CTGTCTCG GGCTAGCTACAACGA GCCCGACC | 7101 |
| 9291 | GGCACGAG A CAGGCUGU | 2305 | ACAGCCTG GGCTAGCTACAACGA CTCGTGCC | 7102 |
| 9287 | CGAGACAG G CUGUGAUA | 2306 | TATCACAG GGCTAGCTACAACGA CTGTCTCG | 7103 |
| 9284 | GACAGGCU G UGAUACAC | 2307 | GTGTATCA GGCTAGCTACAACGA AGCCTGTC | 7104 |
| 9281 | AGGCUGUG A UACACGUC | 2308 | GACGTGTA GGCTAGCTACAACGA CACAGCCT | 7105 |
| 9279 | GCUGUGAU A CACGUCUC | 2309 | GAGACGTG GGCTAGCTACAACGA ATCACAGC | 7106 |
| 9277 | UGUGAUAC A CGUCUCCC | 2310 | GGGAGACG GGCTAGCTACAACGA GTATCACA | 7107 |
| 9275 | UGAUACAC G UCUCCCCC | 2311 | GGGGGAGA GGCTAGCTACAACGA GTGTATCA | 7108 |
| 9266 | UCUCCCCC G CUGUAGCC | 2312 | GGCTACAG GGCTAGCTACAACGA GGGGGAGA | 7109 |
| 9263 | CCCCCGCU G UAGCCAGC | 2313 | GCTGGCTA GGCTAGCTACAACGA AGCGGGGG | 7110 |
| 9260 | CCGCUGUA G CCAGCAAC | 2314 | GTTGCTGG GGCTAGCTACAACGA TACAGCGG | 7111 |
| 9256 | UGUAGCCA G CAACGAAC | 2315 | GTTCGTTG GGCTAGCTACAACGA TGGCTACA | 7112 |
| 9253 | AGCCAGCA A CGAACCAG | 2316 | CTGGTTCG GGCTAGCTACAACGA TGCTGGCT | 7113 |
| 9249 | AGCAACGA A CCAGUUGG | 2317 | CCAACTGG GGCTAGCTACAACGA TCGTTGCT | 7114 |
| 9245 | ACGAACCA G UUGGACAA | 2318 | TTGTCCAA GGCTAGCTACAACGA TGGTTCGT | 7115 |
| 9240 | CCAGUUGG A CAAGUCCA | 2319 | TGGACTTG GGCTAGCTACAACGA CCAACTGG | 7116 |
| 9236 | UUGGACAA G UCCAACUG | 2320 | CAGTTGGA GGCTAGCTACAACGA TTGTCCAA | 7117 |
| 9231 | CAAGUCCA A CUGAGACG | 2321 | CGTCTCAG GGCTAGCTACAACGA TGGACTTG | 7118 |
| 9225 | CAACUGAG A CGCAGCUG | 2322 | CAGCTGCG GGCTAGCTACAACGA CTCAGTTG | 7119 |
| 9223 | ACUGAGAC G CAGCUGGG | 2323 | CCCAGCTG GGCTAGCTACAACGA GTCTCAGT | 7120 |
| 9220 | GAGACGCA G CUGGGAUU | 2324 | AATCCCAG GGCTAGCTACAACGA TGCGTCTC | 7121 |
| 9214 | GGAUUGGA G UGAGUUUG | 2325 | CACTCCAA GGCTAGCTACAACGA CCCAGCTG | 7122 |
| 9208 | GGAUUGGA G UGAGUUUG | 2326 | CAAACTCA GGCTAGCTACAACGA TCCAATCC | 7123 |
| 9204 | UGGAGUGA G UUUGAGUU | 2327 | AACTCAAA GGCTAGCTACAACGA TCACTCCA | 7124 |
| 9198 | GAGUUUGA G UUUGGUCU | 2328 | AGACCAAA GGCTAGCTACAACGA TCAAACTC | 7125 |
| 9193 | UGAGUUUG G UCUUUACU | 2329 | AGTAAAGA GGCTAGCTACAACGA CAAACTCA | 7126 |
| 9187 | UGGUCUUU A CUGCCCAG | 2330 | CTGGGCAG GGCTAGCTACAACGA AAAGACCA | 7127 |
| 9184 | CAAGCCCA G CCCAGUUG | 2331 | CAACTGGG GGCTAGCTACAACGA AGTAAAGA | 7128 |
| 9179 | ACUGCCCA G UUGAAGAG | 2332 | CTCTTCAA GGCTAGCTACAACGA TGGGCAGT | 7129 |
| 9170 | UUGAAGAG G UACCUGCC | 2333 | GGCAGGTA GGCTAGCTACAACGA CTCTTCAA | 7130 |
| 9168 | GAAGAGGU A CCUGCCAC | 2334 | GTGGCAGG GGCTAGCTACAACGA ACCTCTTC | 7131 |
| 9164 | AGGUACCU G CCACAGGU | 2335 | ACCTGTGG GGCTAGCTACAACGA AGGTACCT | 7132 |
| 9161 | UACCUGCC A CAGGUGGC | 2336 | GCCACCTG GGCTAGCTACAACGA GGCAGGTA | 7133 |
| 9157 | UGCCACAG G UGGCGGCC | 2337 | GGCCGCCA GGCTAGCTACAACGA CTGTGGCA | 7134 |
| 9154 | CACAGGUG G CGGCCCUC | 2338 | GAGGGCCG GGCTAGCTACAACGA CACCTGTG | 7135 |
| 9151 | AGGUGGCG G CCCUCCCC | 2339 | GGGGAGGG GGCTAGCTACAACGA CGCCACCT | 7136 |
| 9135 | CCCCUGGG A CAGUAGCU | 2340 | AGCTACTG GGCTAGCTACAACGA CCCAGGGG | 7137 |
| 9132 | CUGGGACA G UAGCUUAG | 2341 | CTAAGCTA GGCTAGCTACAACGA TGTCCCAG | 7138 |
| 9129 | GGACAGUA G CUUAGCGC | 2342 | GCGCTAAG GGCTAGCTACAACGA TACTGTCC | 7139 |
| 9124 | GUAGCUUA G CGCGAACA | 2343 | TGTTCGCG GGCTAGCTACAACGA TAAGCTAC | 7140 |
| 9122 | AGCUUAGC G CGAACACU | 2344 | AGTGTTCG GGCTAGCTACAACGA GCTAAGCT | 7141 |
| 9118 | UAGCGCGA A CACUUCUG | 2345 | CAGAAGTG GGCTAGCTACAACGA TCGCGCTA | 7142 |
| 9116 | GCGCGAAC A CUUCUGGC | 2346 | GCCAGAAG GGCTAGCTACAACGA GTTCGCGC | 7143 |
| 9109 | CACUUCUG G CCCGAUGU | 2347 | ACATCGGG GGCTAGCTACAACGA CAGAAGTG | 7144 |
| 9104 | CUGGCCCG A UGUCUCCA | 2348 | TGGAGACA GGCTAGCTACAACGA CGGGCCAG | 7145 |
| 9102 | GGCCCGAU G UCUCCAGG | 2349 | CCTGGAGA GGCTAGCTACAACGA ATCGGGCC | 7146 |
| 9094 | GUCUCCAG G UUCGCAAG | 2350 | CTTGCGAA GGCTAGCTACAACGA CTGGAGAC | 7147 |
| 9090 | CCAGGUUC G CAAGGGUG | 2351 | CACCCTTG GGCTAGCTACAACGA GAACCTGG | 7148 |
| 9084 | UCGCAAGG G UGGUACCC | 2352 | GGGTACCA GGCTAGCTACAACGA CCTTGCGA | 7149 |
| 9081 | CAAGGGUG G UACCCCAA | 2353 | TTGGGGTA GGCTAGCTACAACGA CACCCTTG | 7150 |
| 9079 | AGGGUGGU A CCCCAAGU | 2354 | ACTTGGGG GGCTAGCTACAACGA ACCACCCT | 7151 |
| 9072 | UACCCCAA G UUUCCUGA | 2355 | TCAGGAAA GGCTAGCTACAACGA TTGGGGTA | 7152 |
| 9062 | UUCCUGAG G CAUGAUGC | 2356 | GCATCATG GGCTAGCTACAACGA CTCAGGAA | 7153 |
| 9060 | CCUGAGGC A UGAUGCCA | 2357 | TGGCATCA GGCTAGCTACAACGA GCCTCAGG | 7154 |
| 9057 | GAGGCAUG A UGCCACCC | 2358 | GGGTGGCA GGCTAGCTACAACGA CATGCCTC | 7155 |
| 9055 | GGCAUGAU G CCACCCUA | 2359 | TAGGGTGG GGCTAGCTACAACGA ATCATGCC | 7156 |
| 9052 | AUGAUGCC A CCCUAUUG | 2360 | CAATAGGG GGCTAGCTACAACGA GGCATCAT | 7157 |
| 9047 | GCCACCCU A UUGAUUUC | 2361 | GAAATCAA GGCTAGCTACAACGA AGGGTGGC | 7158 |
| 9043 | CCCUAUUG A UUUCACCU | 2362 | AGGTGAAA GGCTAGCTACAACGA CAATAGGG | 7159 |
| 9038 | UUGAUUUC A CCUGGGGA | 2363 | TCCCCAGG GGCTAGCTACAACGA GAAATCAA | 7160 |
| 9029 | CCUGGGGA G UAACUAUG | 2364 | CATAGTTA GGCTAGCTACAACGA TCCCCAGG | 7161 |
| 9026 | GGGGAGUA A CUAUGGAG | 2365 | CTCCATAG GGCTAGCTACAACGA TACTCCCC | 7162 |
| 9023 | GAGUAACU A UGGAGUGA | 2366 | TCACTCCA GGCTAGCTACAACGA AGTTACTC | 7163 |
| 9018 | ACUAUGGA G UGAAAAUG | 2367 | CATTTTCA GGCTAGCTACAACGA TCCATAGT | 7164 |
| 9012 | GAGUGAAA A UGCGCUAA | 2368 | TTAGCGCA GGCTAGCTACAACGA TTTCACTC | 7165 |
| 9010 | GUGAAAAU G CGCUAAGA | 2369 | TCTTAGCG GGCTAGCTACAACGA ATTTTCAC | 7166 |
| 9008 | GAAAAUGC G CUAAGACC | 2370 | GGTCTTAG GGCTAGCTACAACGA GCATTTTC | 7167 |
| 9002 | GCGCUAAG A CCAUGGAG | 2371 | CTCCATGG GGCTAGCTACAACGA CTTAGCGC | 7168 |
| 8999 | CUAAGACC A UGGAGUCG | 2372 | CGACTCCA GGCTAGCTACAACGA GGTCTTAG | 7169 |
| 8994 | ACCAUGGA G UCGCUGAA | 2373 | TTCAGCGA GGCTAGCTACAACGA TCCATGGT | 7170 |
| 8991 | AUGGAGUC G CUGAAUGA | 2374 | TCATTCAG GGCTAGCTACAACGA GACTCCAT | 7171 |
| 8986 | GUCGCUGA A UGAUCUGA | 2375 | TCAGATCA GGCTAGCTACAACGA TCAGCGAC | 7172 |
| 8983 | GCUGAAUG A UCUGAGGU | 2376 | ACCTCAGA GGCTAGCTACAACGA CATTCAGC | 7173 |
| 8976 | GAUCUGAG G UAGGUCAA | 2377 | TTGACCTA GGCTAGCTACAACGA CTCAGATC | 7174 |
| 8972 | UGAGGUAG G UCAAGUGG | 2378 | CCACTTGA GGCTAGCTACAACGA CTACCTCA | 7175 |
| 8967 | UAGGUCAA G UGGCUCAA | 2379 | TTGAGCCA GGCTAGCTACAACGA TTGACCTA | 7176 |
| 8964 | GUCAAGUG G CUCAAUGG | 2380 | CCATTGAG GGCTAGCTACAACGA CACTTGAC | 7177 |
| 8959 | GUGGCUCA A UGGAGUAA | 2381 | TTACTCCA GGCTAGCTACAACGA TGAGCCAC | 7178 |
| 8954 | UCAAUGGA G UAACAAGC | 2382 | GCTTGTTA GGCTAGCTACAACGA TCCATTGA | 7179 |
| 8951 | AUGGAGUA A CAAGCCCC | 2383 | GGGGCTTG GGCTAGCTACAACGA TACTCCAT | 7180 |
| 8947 | AGUAACAA G CCCCGUAG | 2384 | CTACGGGG GGCTAGCTACAACGA TTGTTACT | 7181 |
| 8942 | CAAGCCCC G UAGAUCUG | 2385 | CAGATCTA GGCTAGCTACAACGA GGGGCTTG | 7182 |
| 8938 | CCCCGUAG A UCUGGCAG | 2386 | CTGCCAGA GGCTAGCTACAACGA CTACGGGG | 7183 |
| 8933 | UAGAUCUG G CAGUCUAG | 2387 | CTAGACTG GGCTAGCTACAACGA CAGATCTA | 7184 |
| 8930 | AUCUGGCA G UCUAGGGC | 2388 | GCCCTAGA GGCTAGCTACAACGA TGCCAGAT | 7185 |
| 8923 | AGUCUAGG G CUUUCUCA | 2389 | TGAGAAAG GGCTAGCTACAACGA CCTAGACT | 7186 |
| 8913 | UUUCUCAA G UUGCUCCU | 2390 | AGGAGCAA GGCTAGCTACAACGA TTGAGAAA | 7187 |
| 8910 | CUCAAGUU G CUCCUGGG | 2381 | CCCAGGAG GGCTAGCTACAACGA AACTTGAG | 7188 |
| 8902 | GCUCCUGG G CUAGAAGG | 2392 | CCTTCTAG GGCTAGCTACAACGA CCAGGAGC | 7189 |
| 8893 | CUAGAAGG A UGGAGAAG | 2393 | CTTCTCCA GGCTAGCTACAACGA TTCTTCTC | 7190 |
| 8882 | GAGAAGAA G UGAGUCAU | 2394 | ATGACTCA GGCTAGCTACAACGA TTCTTCTC | 7191 |
| 8878 | AGAAGUGA G UCAUCAGA | 2395 | TCTGATGA GGCTAGCTACAACGA TCACTTCT | 7192 |
| 8875 | AGUGAGUC A UCAGAAUC | 2396 | GATTCTGA GGCTAGCTACAACGA GACTCACT | 7193 |
| 8869 | UCAUCAGA A UCAUCCUU | 2397 | AAGGATGA GGCTAGCTACAACGA TCTGATGA | 7194 |
| 8866 | UCAGAAUC A UCCUUACC | 2398 | GGTAAGGA GGCTAGCTACAACGA GATTCTGA | 7195 |
| 8860 | UCAUCCUU A CCCAUAGA | 2399 | TCTATGGG GGCTAGCTACAACGA AAGGATGA | 7196 |
| 8856 | CCUUACCC A UAGAGUGG | 2400 | CCACTCTA GGCTAGCTACAACGA GGGTAAGG | 7197 |
| 8851 | CCCAUAGA G UGGGUGCA | 2401 | TGCACCCA GGCTAGCTACAACGA TCTATGGG | 7198 |
| 8847 | UAGAGUGG G UGCAAACA | 2402 | TCATCATG GGCTAGCTACAACGA TTGCACCC | 7199 |
| 8845 | GAGUGGGU G CAAACAUG | 2403 | CATGTTTG GGCTAGCTACAACGA ACCCACTC | 7200 |
| 8841 | GGGUGCAA A CAUGAUGA | 2404 | TCATCATG GGCTAGCTACAACGA TTGCACCC | 7201 |
| 8839 | GUGCAAAC A UGAUGAUG | 2405 | CATCATCA GGCTAGCTACAACGA GTTTGCAC | 7202 |
| 8836 | CAAACAUG A UGAUGUUG | 2406 | CAACATCA GGCTAGCTACAACGA CATGTTTG | 7203 |
| 8833 | ACAUGAUG A UGUUGCCU | 2407 | AGGCAACA GGCTAGCTACAACGA CATCATGT | 7204 |
| 8831 | AUGAUGAU G UUGCCUAG | 2408 | CTAGGCAA GGCTAGCTACAACGA ATCATCAT | 7205 |
| 8828 | AUGAUGUU G CUUAGCCA | 2409 | TGGCTAGG GGCTAGCTACAACGA AACATCAT | 7206 |
| 8823 | GUUGCCAU G UUGACUGG | 2410 | ACTCCTGG GGCTAGCTACAACGA TAGGCAAC | 7207 |
| 8816 | AGCCAGGA G UUGACUGG | 2411 | CCAGTCAA GGCTAGCTACAACGA TCCTGGCT | 7208 |
| 8812 | AGGAGUUG A CUGGAGUG | 2412 | CACTCCAG GGCTAGCTACAACGA CAACTCCT | 7209 |
| 8806 | UGACUGGA G UGCUUCUA | 2413 | TAGAAGCA GGCTAGCTACAACGA TCCAGTCA | 7210 |
| 8804 | ACUGGAGU G CUUCUAGC | 2414 | GCTAGAAG GGCTAGCTACAACGA ACTCCAGT | 7211 |
| 8797 | UGCUUCUA G CUGUCUCC | 2415 | GGAGACAG GGCTAGCTACAACGA TAGAAGCA | 7212 |
| 8794 | UUCUAGCU G UCUCCCAC | 2416 | GTGGGAGA GGCTAGCTACAACGA AGCTAGAA | 7213 |
| 8787 | UGUCUCCC A CGCAGCCC | 2417 | GGGCTGCG GGCTAGCTACAACGA GGGAGACA | 7214 |
| 8785 | UCUCCCAC G CAGCCCGC | 2418 | GCGGGCTG GGCTAGCTACAACGA GTGGGAGA | 7215 |
| 8782 | CCCACGCA G CCCGCGCA | 2419 | TGCGCGGG GGCTAGCTACAACGA TGCGTGGG | 7216 |
| 8778 | CGCAGCCC G CGCAAGGG | 2420 | CCCTTGCG GGCTAGCTACAACGA GGGCTGCG | 7217 |
| 8776 | CAGCCCGC G CAAGGGGG | 2421 | CCCCCTTG GGCTAGCTACAACGA GCGGGCTG | 7218 |
| 8767 | CAAGGGGG G UGGUGGGG | 2422 | CCCCACCA GGCTAGCTACAACGA CCCCCTTG | 7219 |
| 8764 | GGGGGGUG G UGGGGUCA | 2423 | TGACCCCA GGCTAGCTACAACGA CACCCCCC | 7220 |
| 8759 | GUGGUGGG G UCACGGGU | 2424 | ACCCGTGA GGCTAGCTACAACGA CCCACCAC | 7221 |
| 8756 | GUGGGGUC A CGGGUGAG | 2425 | CTCACCCG GGCTAGCTACAACGA GACCCCAC | 7222 |
| 8752 | GGUCACGG G UGAGGUAG | 2426 | CTACCTCA GGCTAGCTACAACGA CCGTGACC | 7223 |
| 8747 | CGGGUGAG G UAGUACAC | 2427 | GTGTACTA GGCTAGCTACAACGA CTCACCCG | 7224 |
| 8744 | GUGAGGUA G UACACCCU | 2428 | AGGGTGTA GGCTAGCTACAACGA TACCTCAC | 7225 |
| 8742 | GAGGUAGU A CACCCUUU | 2429 | AAAGGGTG GGCTAGCTACAACGA ACTACCTC | 7226 |
| 8740 | GGUAGUAC A CCCUUUUG | 2430 | CAAAAGGG GGCTAGCTACAACGA GTACTACC | 7227 |
| 8732 | ACCCUUUU G CCAGAUGC | 2431 | GCATCTGG GGCTAGCTACAACGA AAAAGGGT | 7228 |
| 8727 | UUUGCCAG A UGCAUCGU | 2432 | ACGATGCA GGCTAGCTACAACGA CTGGCAAA | 7229 |
| 8725 | UGCCAGAU G CAUCGUGU | 2433 | ACACGATG GGCTAGCTACAACGA ATCTGGCA | 7230 |
| 8723 | CCAGAUGC A UCGUGUGC | 2434 | GCACACGA GGCTAGCTACAACGA GCATCTGG | 7231 |
| 8720 | GAUGCAUC G UGUGCAAC | 2435 | GTTGCACA GGCTAGCTACAACGA GATGCATC | 7232 |
| 8718 | UGCAUCGU G UGCAACUG | 2436 | CAGTTGCA GGCTAGCTACAACGA ACGATGCA | 7233 |
| 8716 | CAUCGUGU G CAACUGAU | 2437 | ATCAGTTG GGCTAGCTACAACGA ACACGATG | 7234 |
| 8713 | CGUGUGCA A CUGAUACG | 2438 | CGTATCAG GGCTAGCTACAACGA TGCACACG | 7235 |
| 8709 | UGCAACUG A UACGUUGG | 2439 | CCAACGTA GGCTAGCTACAACGA CAGTTGCA | 7236 |
| 8707 | CAACUGAU A CGUUGGAG | 2440 | CTCCAACG GGCTAGCTACAACGA ATCAGTTG | 7237 |
| 8705 | ACUGAUAC G UUGGAGGA | 2441 | TCCTCCAA GGCTAGCTACAACGA GTATCAGT | 7238 |
| 8696 | UUGGAGGA G CAUGAUGU | 2442 | ACATCATG GGCTAGCTACAACGA TCCTCCAA | 7239 |
| 8694 | GGAGGAGC A UGAUGUUA | 2443 | TAACATCA GGCTAGCTACAACGA GCTCCTCC | 7240 |
| 8691 | GGAGCAUG A UGUUAUCA | 2444 | TGATAACA GGCTAGCTACAACGA CATGCTCC | 7241 |
| 8689 | AGCAUGAU G UUAUCAAC | 2445 | GTTGATAA GGCTAGCTACAACGA ATCATGCT | 7242 |
| 8686 | AUGAUGUU A UCAACUCC | 2446 | GGAGTTGA GGCTAGCTACAACGA AACATCAT | 7243 |
| 8682 | UGUUAUCA A CUCCAAGU | 2447 | ACTTGGAG GGCTAGCTACAACGA TGATAACA | 7244 |
| 8675 | AACUCCAA G UCGUAUUC | 2448 | GAATACGA GGCTAGCTACAACGA TTGGAGTT | 7245 |
| 8672 | UCCAAGUC G UAUUCCGG | 2449 | CCGGAATA GGCTAGCTACAACGA GACTTGGA | 7246 |
| 8670 | CAAGUCGU A UUCCGGUU | 2450 | AACCGGAA GGCTAGCTACAACGA ACGACTTG | 7247 |
| 8664 | GUAUUCCG G UUGGGGCG | 2451 | CGCCCCAA GGCTAGCTACAACGA CGGAATAC | 7248 |
| 8658 | CGGUUGGG G CGGGUCCC | 2452 | GGGACCCG GGCTAGCTACAACGA CCCAACCG | 7249 |
| 8654 | UGGGGCGG G UCCCCGGG | 2453 | CCCGGGGA GGCTAGCTACAACGA CCGCCCCA | 7250 |
| 8641 | CGGGGGGG G CAGAGUAC | 2454 | GTACTCTG GGCTAGCTACAACGA CCCCCCCG | 7251 |
| 8636 | GGGGCAGA G UACCUAGU | 2455 | ACTAGGTA GGCTAGCTACAACGA TCTGCCCC | 7252 |
| 8634 | GGCAGAGU A CCUAGUCA | 2456 | TGACTAGG GGCTAGCTACAACGA ACTCTGCC | 7253 |
| 8629 | AGUACCUA G UCAUAGCC | 2457 | GGCTATGA GGCTAGCTACAACGA TAGGTACT | 7254 |
| 8626 | ACCAUGUC A UAGCCUCC | 2458 | GGAGGCTA GGCTAGCTACAACGA GACTAGGT | 7255 |
| 8623 | UAGUCAUA G CCUCCGUG | 2459 | CACGGAGG GGCTAGCTACAACGA TATGACTA | 7256 |
| 8617 | UAGCCUCC G UGAAGACU | 2460 | AGTCTTCA GGCTAGCTACAACGA GGAGGCTA | 7257 |
| 8611 | CCGUGAAG A CUCGUAGG | 2461 | CCTACGAG GGCTAGCTACAACGA CTTCACGG | 7258 |
| 8607 | GAAGACUC G UAGGCUCG | 2462 | CGAGCCTA GGCTAGCTACAACGA GAGTCTTC | 7259 |
| 8603 | ACUCGUAG G CUCGCCGC | 2463 | GCGGCGAG GGCTAGCTACAACGA CTACGAGT | 7260 |
| 8599 | GUAGGCUC G CCGCGUCC | 2464 | GGACGCGG GGCTAGCTACAACGA GAGCCTAC | 7261 |
| 8596 | GGCUCGCC G CGUCCUCU | 2465 | AGAGGACG GGCTAGCTACAACGA GGCGAGCC | 7262 |
| 8594 | CUCGCCGC G UCCUCUUG | 2466 | CAAGAGGA GGCTAGCTACAACGA GCGGCGAG | 7263 |
| 8584 | CCUCUUGG G UCCCCGCA | 2467 | TGCGGGGA GGCTAGCTACAACGA CCAAGAGG | 7264 |
| 8578 | GGGUCCCC G CACUUUCA | 2468 | TGAAAGTG GGCTAGCTACAACGA GGGGACCC | 7265 |
| 8576 | GUCCCCGC A CUUUCACA | 2469 | TGTGAAAG GGCTAGCTACAACGA GCGGGGAC | 7266 |
| 8570 | GCACUUUC A CAGAUAAC | 2470 | GTTATCTG GGCTAGCTACAACGA GAAAGTGC | 7267 |
| 8566 | UUUCACAG A UAACGACC | 2471 | GGTCGTTA GGCTAGCTACAACGA CTGTGAAA | 7268 |
| 8563 | CACAGAUA A CGACCAGG | 2472 | CCTGGTCG GGCTAGCTACAACGA TATCTGTG | 7269 |
| 8560 | AGAUAACG A CCAGGUCG | 2473 | CGACCTGG GGCTAGCTACAACGA CGTTATCT | 7270 |
| 8555 | ACGACCAG G UCGUCUCC | 2474 | GGAGACGA GGCTAGCTACAACGA CTGGTCGT | 7271 |
| 8552 | ACCAGGUC G UCUCCACA | 2475 | TGTGGAGA GGCTAGCTACAACGA GACCTGGT | 7272 |
| 8546 | UCGUCUCC A CACACGAG | 2476 | CTCGTGTG GGCTAGCTACAACGA GGAGACGA | 7273 |
| 8544 | GUCUCCAC A CACGAGCA | 2477 | TGCTCGTG GGCTAGCTACAACGA GTGGAGAC | 7274 |
| 8542 | CUCCACAC A CGAGCAUC | 2478 | GATGCTCG GGCTAGCTACAACGA GTGTGGAG | 7275 |
| 8538 | ACACACGA G CAUCGUGC | 2479 | GCACGATG GGCTAGCTACAACGA TCGTGTGT | 7276 |
| 8536 | ACACGAGC A UCGUGCAG | 2480 | CTGCACGA GGCTAGCTACAACGA GCTCGTGT | 7277 |
| 8533 | CGAGCAUC G UGCAGUCC | 2481 | GGACTGCA GGCTAGCTACAACGA GATGCTCG | 7278 |
| 8531 | AGCAUCGU G CAGUCCUG | 2482 | CAGGACTG GGCTAGCTACAACGA ACGATGCT | 7279 |
| 8528 | AUCGUGCA G UCCUGGAG | 2483 | CTCCAGGA GGCTAGCTACAACGA TGCACGAT | 7280 |
| 8520 | GUCCUGGA G CUUCGCAG | 2484 | CTGCGAAG GGCTAGCTACAACGA TCCAGGAC | 7281 |
| 8515 | GGAGCUUC G CAGCUCGA | 2485 | TCGAGCTG GGCTAGCTACAACGA GAAGCTCC | 7282 |
| 8512 | GCUUCGCA G CUCGACAG | 2486 | CTGTCGAG GGCTAGCTACAACGA TGCGAAGC | 7283 |
| 8507 | GCAGCUCG A CAGGCCGC | 2487 | GCGGCCTG GGCTAGCTACAACGA CGAGCTGC | 7284 |
| 8503 | CUCGACAG G CCGCAGAG | 2488 | CTCTGCGG GGCTAGCTACAACGA CTGTCGAG | 7285 |
| 8500 | GACAGGCC G CAGAGGCU | 2489 | AGCCTCTG GGCTAGCTACAACGA GGCCTGTC | 7286 |
| 8494 | CCGCAGAG G CUUUCAAG | 2490 | CTTGAAAG GGCTAGCTACAACGA CTCTGCGG | 7287 |
| 8486 | GCUUUCAA G UAACAUGU | 2491 | ACATGTTA GGCTAGCTACAACGA TTGAAAGC | 7288 |
| 8483 | UUCAAGUA A CAUGUGAG | 2492 | CTCACATG GGCTAGCTACAACGA TACTTGAA | 7289 |
| 8481 | CAAGUAAC A UGUGAGGG | 2493 | CCCTCACA GGCTAGCTACAACGA GTTACTTG | 7290 |
| 8479 | AGUAACAU G UGAGGGUA | 2494 | TACCCTCA GGCTAGCTACAACGA ATGTTACT | 7291 |
| 8473 | AUGUGAGG G UAUUACCA | 2495 | TGGTAATA GGCTAGCTACAACGA CCTCACAT | 7292 |
| 8471 | GUGAGGGU A UUACCACA | 2496 | TGTGGTAA GGCTAGCTACAACGA ACCCTCAC | 7293 |
| 8468 | AGGGUAUU A CCACAGCU | 2497 | AGCTGTGG GGCTAGCTACAACGA AATACCCT | 7294 |
| 8465 | GUAUUACC A CAGCUGGU | 2498 | ACCAGCTG GGCTAGCTACAACGA GGTAATAC | 7295 |
| 8462 | UUACCACA G CUGGUCGU | 2499 | ACGACCAG GGCTAGCTACAACGA TGTGGTAA | 7296 |
| 8458 | CACAGCUG G UCGUCAGC | 2500 | GCTGACGA GGCTAGCTACAACGA CAGCTGTG | 7297 |
| 8455 | AGCUGGUC G UCAGCACG | 2501 | CGTGCTGA GGCTAGCTACAACGA GACCAGCT | 7298 |
| 8451 | GGUCGUCA G CACGCCGC | 2502 | GCGGCGTG GGCTAGCTACAACGA TGACGACC | 7299 |
| 8449 | UCGUCAGC A CGCCGCUC | 2503 | GAGCGGCG GGCTAGCTACAACGA GCTGACGA | 7300 |
| 8447 | GUCAGCAC G CCGCUCGC | 2504 | GCGAGCGG GGCTAGCTACAACGA GTGCTGAC | 7301 |
| 8444 | AGCACGCC G CUCGCGCG | 2505 | CGCGCGAG GGCTAGCTACAACGA GGCGTGCT | 7302 |
| 8440 | CGCCGCUC G CGCGGCAC | 2506 | GTGCCGCG GGCTAGCTACAACGA GAGCGGCG | 7303 |
| 8438 | CCGCUCGC G CGGCACCG | 2507 | CGGTGCCG GGCTAGCTACAACGA GCGAGCGG | 7304 |
| 8435 | CUCGCGCG G CACCGGCG | 2508 | CGCCGGTG GGCTAGCTACAACGA CGCGCGAG | 7305 |
| 8433 | CGCGCGGC A CCGGCGAU | 2509 | ATCGCCGG GGCTAGCTACAACGA GCCGCGCG | 7306 |
| 8429 | CGGCACCG G CGAUAACC | 2510 | GGTTATCG GGCTAGCTACAACGA CGGTGCCG | 7307 |
| 8426 | CACCGGCG A UAACCGCA | 2511 | TGCGGTTA GGCTAGCTACAACGA CGCCGGTG | 7308 |
| 8423 | CGGCGAUA A CCGCAGUU | 2512 | AACTGCGG GGCTAGCTACAACGA TATCGCCG | 7309 |
| 8420 | CGAUAACC G CAGUUCUG | 2513 | CAGAACTG GGCTAGCTACAACGA GGTTATCG | 7310 |
| 8417 | UAACCGCA G UUCUGCCC | 2514 | GGGCAGAA GGCTAGCTACAACGA TGCGGTTA | 7311 |
| 8412 | GCAGUUCU G CCCUUUUG | 2515 | CAAAAGGG GGCTAGCTACAACGA AGAACTGC | 7312 |
| 8402 | CCUUUUGA A UUAGUCAG | 2516 | CTGACTAA GGCTAGCTACAACGA TCAAAAGG | 7313 |
| 8398 | UUGAAUUA G UCAGAGGA | 2517 | TCCTCTGA GGCTAGCTACAACGA TAATTCAA | 7314 |
| 8390 | GUCAGAGG A CCCCCGAU | 2518 | ATCGGGGG GGCTAGCTACAACGA CCTCTGAC | 7315 |
| 8383 | GACCCCCG A UAUAAAGC | 2519 | GCTTTATA GGCTAGCTACAACGA CGGGGGTC | 7316 |
| 8381 | CCCCCGAU A UAAAGCCG | 2520 | CGGCTTTA GGCTAGCTACAACGA ATCGGGGG | 7317 |
| 8376 | GAUAUAAA G CCGCUCUG | 2521 | CAGAGCGG GGCTAGCTACAACGA TTTATATC | 7318 |
| 8373 | AUAAAGCC G CUCUGUGA | 2522 | TCACAGAG GGCTAGCTACAACGA GGCTTTAT | 7319 |
| 8368 | GCCGCUCU G UGAGCGAC | 2523 | GTCGCTCA GGCTAGCTACAACGA AGAGCGGC | 7320 |
| 8364 | CUCUGUGA G CGACCUUA | 2524 | TAAGGTCG GGCTAGCTACAACGA TCACAGAG | 7321 |
| 8361 | UGUGAGCG A CCUUAUGG | 2525 | CCATAAGG GGCTAGCTACAACGA CGCTCACA | 7322 |
| 8356 | GCGACCUU A UGGCCUGU | 2526 | ACAGGCCA GGCTAGCTACAACGA AAGGTCGC | 7323 |
| 8353 | ACCUUAUG G CCUGUCUG | 2527 | CAGACAGG GGCTAGCTACAACGA CATAAGGT | 7324 |
| 8349 | UAUGGCCU G UCUGGCUU | 2528 | AAGCCAGA GGCTAGCTACAACGA AGGCCATA | 7325 |
| 8344 | CCUGUCUG G CUUCGGGG | 2529 | CCCCGAAG GGCTAGCTACAACGA CAGACAGG | 7326 |
| 8335 | CUUCGGGG G CCAAGUCA | 2530 | TGACTTGG GGCTAGCTACAACGA CCCCGAAG | 7327 |
| 8330 | GGGGCCAA G UCACAACA | 2531 | TGTTGTGA GGCTAGCTACAACGA TTGGCCCC | 7328 |
| 8327 | GCCAAGUC A CAACAUUG | 2532 | CAATGTTG GGCTAGCTACAACGA GACTTGGC | 7329 |
| 8324 | AAGUCACA A CAUUGGUA | 2533 | TACCAATG GGCTAGCTACAACGA TGTGACTT | 7330 |
| 8322 | GUCACAAC A UUGGUAAA | 2534 | TTTACCAA GGCTAGCTACAACGA GTTGTGAC | 7331 |
| 8318 | CAACAUUG G UAAAUUGA | 2535 | TCAATTTA GGCTAGCTACAACGA CAATGTTG | 7332 |
| 8314 | AUUGGUAA A UUGACUCC | 2536 | GGAGTCAA GGCTAGCTACAACGA TTACCAAT | 7333 |
| 8310 | GUAAAUUG A CUCCUCGA | 2537 | TCGAGGAG GGCTAGCTACAACGA CAATTTAC | 7334 |
| 8302 | ACUCCUCG A CACGGAUG | 2538 | CATCCGTG GGCTAGCTACAACGA CGAGGAGT | 7335 |
| 8300 | UCCUCGAC A CGGAUGUC | 2539 | GACATCCG GGCTAGCTACAACGA GTCGAGGA | 7336 |
| 8296 | CGACACGG A UGUCACUC | 2540 | GAGTGACA GGCTAGCTACAACGA CCGTGTCG | 7337 |
| 8294 | ACACGGAU G UCACUCUC | 2541 | GAGAGTGA GGCTAGCTACAACGA ATCCGTGT | 7338 |
| 8291 | CGGAUGUC A CUCUCGGU | 2542 | ACCGAGAG GGCTAGCTACAACGA GACATCCG | 7339 |
| 8284 | CACUCUCG G UGACUGUU | 2543 | AACAGTCA GGCTAGCTACAACGA CGAGAGTG | 7340 |
| 8281 | UCUCGGUG A CUGUUGAG | 2544 | CTCAACAG GGCTAGCTACAACGA CACCGAGA | 7341 |
| 8278 | CGGUGACU G UUGAGUCG | 2545 | CGACTCAA GGCTAGCTACAACGA AGTCACCG | 7342 |
| 8273 | ACUGUUGA G UCGAAACA | 2546 | TGTTTCGA GGCTAGCTACAACGA TCAACAGT | 7343 |
| 8267 | GAGUCGAA A CAGCGGGU | 2547 | ACCCGCTG GGCTAGCTACAACGA TTCGACTC | 7344 |
| 8264 | UCGAAACA G CGGGUGUC | 2548 | GACACCCG GGCTAGCTACAACGA TGTTTCGA | 7345 |
| 8260 | AACAGCGG G UGUCAUAU | 2549 | ATATGACA GGCTAGCTACAACGA CCGCTGTT | 7346 |
| 8258 | CAGCGGGU G UCAUAUGC | 2550 | GCATATGA GGCTAGCTACAACGA ACCCGCTG | 7347 |
| 8255 | CGGGUGUC A UAUGCAAA | 2551 | TTTGCATA GGCTAGCTACAACGA GACACCCG | 7348 |
| 8253 | GGUGUCAU A UGCAAAGC | 2552 | GCTTTGCA GGCTAGCTACAACGA ATGACACC | 7349 |
| 8251 | UGUCAUAU G CAAAGCCC | 2553 | GGGCTTTG GGCTAGCTACAACGA ATATGACA | 7350 |
| 8246 | UAUGCAAA G CCCAUAGG | 2554 | CCTATGGG GGCTAGCTACAACGA TTTGCATA | 7351 |
| 8242 | CAAAGCCC A UAGGGCAU | 2555 | ATGCCCTA GGCTAGCTACAACGA GGGCTTTG | 7352 |
| 8237 | CCCAUAGG G CAUUUCUU | 2556 | AAGAAATG GGCTAGCTACAACGA CCTATGGG | 7353 |
| 8235 | CAUAGGGC A UUUCUUUG | 2557 | CAAAGAAA GGCTAGCTACAACGA GCCCTATG | 7354 |
| 8226 | UUUCUUUG A UUUCCAGG | 2558 | CCTGGAAA GGCTAGCTACAACGA CAAAGAAA | 7355 |
| 8218 | AUUUCCAG G CAUUCACC | 2559 | GGTGAATG GGCTAGCTACAACGA CTGGAAAT | 7356 |
| 8216 | UUCCAGGC A UUCACCAG | 2560 | CTGGTGAA GGCTAGCTACAACGA GCCTGGAA | 7357 |
| 8212 | AGGCAUUC A CCAGGAAC | 2561 | GTTCCTGG GGCTAGCTACAACGA GAATGCCT | 7358 |
| 8205 | CACCAGGA A CUCAACCC | 2562 | GGGTTGAG GGCTAGCTACAACGA TCCTGGTG | 7359 |
| 8200 | GGAACUCA A CCCGCUGC | 2563 | GCAGCGGG GGCTAGCTACAACGA TGAGTTCC | 7360 |
| 8196 | CUCAACCC G CUGCCCAG | 2564 | CTGGGCAG GGCTAGCTACAACGA GGGTTGAG | 7361 |
| 8193 | AACCCGCU G CCCAGGAG | 2565 | CTCCTGGG GGCTAGCTACAACGA AGCGGGTT | 7362 |
| 8183 | CCAGGAGA G UACUGGAA | 2566 | TTCCAGTA GGCTAGCTACAACGA TCTCCTGG | 7363 |
| 8181 | AGGAGAGU A CUGGAAUC | 2567 | GATTCCAG GGCTAGCTACAACGA ACTCTCCT | 7364 |
| 8175 | GUACUGGA A UCCGUAUG | 2568 | CATACGGA GGCTAGCTACAACGA TCCAGTAC | 7365 |
| 8171 | UGGAAUCC G UAUGAAGA | 2569 | TCTTCATA GGCTAGCTACAACGA GGATTCCA | 7366 |
| 8169 | GAAUCCGU A UGAAGAGC | 2570 | GCTCTTCA GGCTAGCTACAACGA ACGGATTC | 7367 |
| 8162 | UAUGAAGA G CCCAUCAC | 2571 | GTGATGGG GGCTAGCTACAACGA TCTTCATA | 7368 |
| 8158 | AAGAGCCC A UCACGGCC | 2572 | GGCCGTGA GGCTAGCTACAACGA GGGCTCTT | 7369 |
| 8155 | AGCCCAUC A CGGCCUGA | 2573 | TCAGGCCG GGCTAGCTACAACGA GATGGGCT | 7370 |
| 8152 | CCAUCACG G CCUGAGGA | 2574 | TCCTCAGG GGCTAGCTACAACGA CGTGATGG | 7371 |
| 8140 | GAGGAAGG G UGGAGACC | 2575 | GGTCTCCA GGCTAGCTACAACGA CCTTCCTC | 7372 |
| 8134 | GGGUGGAG A CCACGUCG | 2576 | CGACGTGG GGCTAGCTACAACGA CTCCACCC | 7373 |
| 8131 | UGGAGACC A CGUCGUAA | 2577 | TTACGACG GGCTAGCTACAACGA GGTCTCCA | 7374 |
| 8129 | GAGACCAC G UCGUAAAG | 2578 | CTTTACGA GGCTAGCTACAACGA GTGGTCTC | 7375 |
| 8126 | ACCACGUC G UAAAGGGC | 2579 | GCCCTTTA GGCTAGCTACAACGA GACGTGGT | 7376 |
| 8119 | CGUAAAGG G CCAUUUUC | 2580 | CGTGTGTG GGCTAGCTACAACGA GAGAAAAT | 7377 |
| 8116 | AAAGGGCC A UUUUCUCG | 2581 | CGAGAAAA GGCTAGCTACAACGA GGCCCTTT | 7378 |
| 8108 | AUUUUCUC G CACACACG | 2582 | CGTGTGTG GGCTAGCTACAACGA GAGAAAAT | 7379 |
| 8106 | UUUCUCGC A CACACGAA | 2583 | TTCGTGTG GGCTAGCTACAACGA GCGAGAAA | 7380 |
| 8104 | UCUCGCAC A CACGAACC | 2584 | GGTTCGTG GGCTAGCTACAACGA GTGCGAGA | 7381 |
| 8102 | UCGCACAC A CGAACCCC | 2585 | GGGGTTCG GGCTAGCTACAACGA GTGTGCGA | 7382 |
| 8098 | ACACACGA A CCCCCAAG | 2586 | CTTGGGGG GGCTAGCTACAACGA TCGTGTGT | 7383 |
| 8090 | ACCCCCAA G UCUGGGAA | 2587 | TTCCCAGA GGCTAGCTACAACGA TTGGGGGT | 7384 |
| 8082 | GUCUGGGA A CACGAUAA | 2588 | TTATCGTG GGCTAGCTACAACGA TCCCAGAC | 7385 |
| 8080 | CUGGGAAC A CGAUAAGG | 2589 | CCTTATCG GGCTAGCTACAACGA GTTCCCAG | 7386 |
| 8077 | GGAACACG A UAAGGCGA | 2590 | TCGCCTTA GGCTAGCTACAACGA CGTGTTCC | 7387 |
| 8072 | ACGAUAAG G CGAGCUGG | 2591 | CCAGCTCG GGCTAGCTACAACGA CTTATCGT | 7388 |
| 8068 | UAAGGCGA G CUGGCUUG | 2592 | CAAGCCAG GGCTAGCTACAACGA TCGCCTTA | 7389 |
| 8064 | GCGAGCUG G CUUGCGGC | 2593 | GCCGCAAG GGCTAGCTACAACGA CAGCTCGC | 7390 |
| 8060 | GCUGGCUU G CGGCCUCC | 2594 | GGAGGCCG GGCTAGCTACAACGA AAGCCAGC | 7391 |
| 8057 | GGCUUGCG G CCUCCUUU | 2595 | AAAGGAGG GGCTAGCTACAACGA CGCAAGCC | 7392 |
| 8043 | UUUCUCUG G UUGGACGC | 2596 | GCGTCCAA GGCTAGCTACAACGA CAGAGAAA | 7393 |
| 8038 | CUGGUUGG A CGCAGAAA | 2597 | TTTCTGCG GGCTAGCTACAACGA CCAACCAG | 7394 |
| 8036 | GGUUGGAC G CAGAAAAC | 2598 | GTTTTCTG GGCTAGCTACAACGA GTCCAACC | 7395 |
| 8029 | CGCAGAAA A CCUCAUUU | 2599 | AAATGAGG GGCTAGCTACAACGA TTTCTGCG | 7396 |
| 8024 | AAAACCUC A UUUUUUGC | 2600 | GCAAAAAA GGCTAGCTACAACGA GAGGTTTT | 7397 |
| 8017 | CAUUUUUU G CCAUGAUG | 2601 | CATCATGG GGCTAGCTACAACGA AAAAAATG | 7398 |
| 8014 | UUUUUGCC A UGAUGGUG | 2602 | CACCATCA GGCTAGCTACAACGA GGCAAAAA | 7399 |
| 8011 | UUGCCAUG A UGGUGGUA | 2603 | TACCACCA GGCTAGCTACAACGA CATGGCAA | 7400 |
| 8008 | CCAUGAUG G UGGUAUCA | 2604 | TGATACCA GGCTAGCTACAACGA CATCATGG | 7401 |
| 8005 | UGAUGGUG G UAUCAAUU | 2605 | AATTGATA GGCTAGCTACAACGA CACCATCA | 7402 |
| 8003 | AUGGUGGU A UCAAUUGG | 2606 | CCAATTGA GGCTAGCTACAACGA ACCACCAT | 7403 |
| 7999 | UGGUAUCA A UUGGUGUC | 2607 | GACACCAA GGCTAGCTACAACGA TGATACCA | 7404 |
| 7995 | AUCAAUUG G UGUCUCAG | 2608 | CTGAGACA GGCTAGCTACAACGA CAATTGAT | 7405 |
| 7993 | CAAUUGGU G UCUCAGUG | 2609 | CACTGAGA GGCTAGCTACAACGA ACCAATTG | 7406 |
| 7987 | GUGUCUCA G UGUCUUCC | 2610 | GGAAGACA GGCTAGCTACAACGA TGAGACAC | 7407 |
| 7985 | GUCUCAGU G UCUUCCAG | 2611 | CTGGAAGA GGCTAGCTACAACGA ACTGAGAC | 7408 |
| 7977 | GUCUUCCA G CAAGUCCU | 2612 | AGGACTTG GGCTAGCTACAACGA TGGAAGAC | 7409 |
| 7973 | UCCAGCAA G UCCUUCCA | 2613 | TGGAAGGA GGCTAGCTACAACGA TTGCTGGA | 7410 |
| 7965 | GUCCUUCC A CACGGAGC | 2614 | GCTCCGTG GGCTAGCTACAACGA GGAAGGAC | 7411 |
| 7963 | CCUUCCAC A CGGAGCGG | 2615 | CCGCTCCG GGCTAGCTACAACGA GTGGAAGG | 7412 |
| 7958 | CACACGGA G CGGAUGUG | 2616 | CACATCCG GGCTAGCTACAACGA TCCGTGTG | 7413 |
| 7954 | CGGAGCGG A UGUGGUUG | 2617 | CAACCACA GGCTAGCTACAACGA CCGCTCCG | 7414 |
| 7952 | GAGCGGAU G UGGUUGAC | 2618 | GTCAACCA GGCTAGCTACAACGA ATCCGCTC | 7415 |
| 7949 | CGGAUGUG G UUGACGGC | 2619 | GCCGTCAA GGCTAGCTACAACGA CACATCCG | 7416 |
| 7945 | UGUGGUUG A CGGCCCCG | 2620 | CGGGGCCG GGCTAGCTACAACGA CAACCACA | 7417 |
| 7942 | GGUUGACG G CCCCGCUG | 2621 | CAGCGGGG GGCTAGCTACAACGA CGTCAACC | 7418 |
| 7937 | ACGGCCCC G CUGGAUAG | 2622 | CTATCCAG GGCTAGCTACAACGA GGGGCCGT | 7419 |
| 7932 | CCCGCUGG A UAGGUUCC | 2623 | GGAACCTA GGCTAGCTACAACGA CCAGCGGG | 7420 |
| 7928 | CUGGAUAG G UUCCGGAC | 2624 | GTCCGGAA GGCTAGCTACAACGA CTATCCAG | 7421 |
| 7921 | GGUUCCGG A CGUCCUUU | 2625 | AAAGGACG GGCTAGCTACAACGA CCGGAACC | 7422 |
| 7919 | UUCCGGAC G UCCUUUGC | 2626 | GCAAAGGA GGCTAGCTACAACGA GTCCGGAA | 7423 |
| 7912 | CGUCCUUU G CCCCAUAA | 2627 | TTATGGGG GGCTAGCTACAACGA AAAGGACG | 7424 |
| 7907 | UUUGCCCC A UAACCAAA | 2628 | TTTGGTTA GGCTAGCTACAACGA GGGGCAAA | 7425 |
| 7904 | GCCCCAUA A CCAAAUUU | 2629 | AAATTTGG GGCTAGCTACAACGA TATGGGGC | 7426 |
| 7899 | AUAACCAA A UUUGGACC | 2630 | GGTCCAAA GGCTAGCTACAACGA TTGGTTAT | 7427 |
| 7893 | AAAUUUGG A CCUGGCCG | 2631 | CGGCCAGG GGCTAGCTACAACGA CCAAATTT | 7428 |
| 7888 | UGGACCUG G CCGAAUGU | 2632 | ACATTCGG GGCTAGCTACAACGA CAGGTCCA | 7429 |
| 7883 | CUGGCCGA A UGUGGGGG | 2633 | CCCCCACA GGCTAGCTACAACGA TCGGCCAG | 7430 |
| 7881 | GGCCGAAU G UGGGGGCG | 2634 | CGCCCCCA GGCTAGCTACAACGA ATTCGGCC | 7431 |
| 7875 | AUGUGGGG G CGUCAGUC | 2635 | GACTGACG GGCTAGCTACAACGA CCCCACAT | 7432 |
| 7873 | GUGGGGGC G UCAGUCUG | 2636 | CAGACTGA GGCTAGCTACAACGA GCCCCCAC | 7433 |
| 7869 | GGGCGUCA G UCUGCAGG | 2637 | CCTGCAGA GGCTAGCTACAACGA TGACGCCC | 7434 |
| 7865 | GUCAGUCU G CAGGCUUC | 2638 | GAAGCCTG GGCTAGCTACAACGA AGACTGAC | 7435 |
| 7861 | GUCUGCAG G CUUCCUCU | 2639 | AGAGGAAG GGCTAGCTACAACGA CTGCAGAC | 7436 |
| 7852 | CUUCCUCU A CGGAUAGA | 2640 | TCTATCCG GGCTAGCTACAACGA AGAGGAAG | 7437 |
| 7848 | CUCUACGG A UAGAAGUU | 2641 | AACTTCTA GGCTAGCTACAACGA CCGTAGAG | 7438 |
| 7842 | GGAUAGAA G UUUAGCCU | 2642 | AGGCTAAA GGCTAGCTACAACGA TTCTATCC | 7439 |
| 7837 | GAAGUUUA G CCUUAACU | 2643 | AGTTAAGG GGCTAGCTACAACGA TAAACTTC | 7440 |
| 7831 | UAGCCUUA A CUGUGGAC | 2644 | GTCCACAG GGCTAGCTACAACGA TAAGGCTA | 7441 |
| 7828 | CCUUAACU G UGGACGCC | 2645 | GGCGTCCA GGCTAGCTACAACGA AGTTAAGG | 7442 |
| 7824 | AACUGUGG A CGCCUUCG | 2646 | CGAAGGCG GGCTAGCTACAACGA CCACAGTT | 7443 |
| 7822 | CUGUGGAC G CCUUCGCC | 2647 | GGCGAAGG GGCTAGCTACAACGA GTCCACAG | 7444 |
| 7816 | ACGCCUUC G CCUUCAUC | 2648 | GATGAAGG GGCTAGCTACAACGA GAAGGCGT | 7445 |
| 7810 | UCGCCUUC A UCUCCUUG | 2649 | CAAGGAGA GGCTAGCTACAACGA GAAGGCGA | 7446 |
| 7800 | CUCCUUGA G CACGUCCC | 2650 | GGGACGTG GGCTAGCTACAACGA TCAAGGAG | 7447 |
| 7798 | CCUUGAGC A CGUCCCGG | 2651 | CCGGGACG GGCTAGCTACAACGA GCTCAAGG | 7448 |
| 7796 | UUGAGCAC G UCCCGGUA | 2652 | TACCGGGA GGCTAGCTACAACGA GTGCTCAA | 7449 |
| 7790 | ACGUCCCG G UAGUGGUC | 2653 | GACCACTA GGCTAGCTACAACGA CGGGACGT | 7450 |
| 7787 | UCCCGGUA G UGGUCGUC | 2654 | GACGACCA GGCTAGCTACAACGA TACCGGGA | 7451 |
| 7784 | CGGUAGUG G UCGUCCAG | 2655 | CTGGACGA GGCTAGCTACAACGA CACTACCG | 7452 |
| 7781 | UAGUGGUC G UCCAGGAC | 2656 | GTCCTGGA GGCTAGCTACAACGA GACCACTA | 7453 |
| 7774 | CGUCCAGG A CUUGCAGU | 2657 | ACTGCAAG GGCTAGCTACAACGA CCTGGACG | 7454 |
| 7770 | CAGGACUU G CAGUCUGU | 2658 | ACAGACTG GGCTAGCTACAACGA AAGTCCTG | 7455 |
| 7767 | GACUUGCA G UCUGUCAA | 2659 | TTGACAGA GGCTAGCTACAACGA TGCAAGTC | 7456 |
| 7763 | UGCAGUCU G UCAAAGGU | 2660 | ACCTTTGA GGCTAGCTACAACGA AGACTGCA | 7457 |
| 7756 | UGUCAAAG G UGACCUUC | 2661 | GAAGGTCA GGCTAGCTACAACGA CTTTGACA | 7458 |
| 7753 | CAAAGGUG A CCUUCUUC | 2662 | GAAGAAGG GGCTAGCTACAACGA CACCTTTG | 7459 |
| 7743 | CUUCUUCU G CCGCUGGC | 2663 | GCCAGCGG GGCTAGCTACAACGA AGAAGAAG | 7460 |
| 7740 | CUUCUGCC G CUGGCUUG | 2664 | CAAGCCAG GGCTAGCTACAACGA GGCAGAAG | 7461 |
| 7736 | UGCCGCUG G CUUGCGCU | 2665 | AGCGCAAG GGCTAGCTACAACGA CAGCGGCA | 7462 |
| 7732 | GCUGGCUU G CGCUGCGA | 2666 | TCGCAGCG GGCTAGCTACAACGA AAGCCAGC | 7463 |
| 7730 | UGGCUUGC G CUGCGAGA | 2667 | TCTCGCAG GGCTAGCTACAACGA GCAAGCCA | 7464 |
| 7727 | CUUGCGCU G CGAGAUGU | 2668 | ACATCTCG GGCTAGCTACAACGA AGCGCAAG | 7465 |
| 7722 | GCUGCGAG A UGUUGUAG | 2669 | CTACAACA GGCTAGCTACAACGA CTCGCAGC | 7466 |
| 7720 | UGCGAGAU G UUGUAGCG | 2670 | CGCTACAA GGCTAGCTACAACGA ATCTCGCA | 7467 |
| 7717 | GAGAUGUU G UAGCGUAG | 2671 | CTACGCTA GGCTAGCTACAACGA AACATCTC | 7468 |
| 7714 | AUGUUGUA G CGUAGACC | 2672 | GGTCTACG GGCTAGCTACAACGA TACAACAT | 7469 |
| 7712 | GUUGUAGC G UAGACCAU | 2673 | ATGGTCTA GGCTAGCTACAACGA GCTACAAC | 7470 |
| 7708 | UAGCGUAG A CCAUGUUG | 2674 | CAACATGG GGCTAGCTACAACGA CTACGCTA | 7471 |
| 7705 | CGAUGACC A UGUUGUGG | 2675 | CCACAACA GGCTAGCTACAACGA GGTCTACG | 7472 |
| 7703 | UAGACCAU G UUGUGGUG | 2676 | CACCACAA GGCTAGCTACAACGA ATGGTCTA | 7473 |
| 7700 | ACCAUGUU G UGGUGACG | 2677 | CGTCACCA GGCTAGCTACAACGA AACATGGT | 7474 |
| 7697 | AUGUUGUG G UGACGCAG | 2678 | CTGCGTCA GGCTAGCTACAACGA CACAACAT | 7475 |
| 7694 | GUUGCUCA A CGCGUUGA | 2679 | TTGCTGCG GGCTAGCTACAACGA CACCACAA | 7476 |
| 7692 | GUGGUGAC G CAGCAAAG | 2680 | CTTTGCTG GGCTAGCTACAACGA GTCACCAC | 7477 |
| 7689 | GUGACGCA G CAAAGAGU | 2681 | ACTCTTTG GGCTAGCTACAACGA TGCGTCAC | 7478 |
| 7682 | AGCAAAGA G UUGCUCAA | 2682 | TTGAGCAA GGCTAGCTACAACGA TCTTTGCT | 7479 |
| 7679 | AAAGAGUU G CUCAACGC | 2683 | GCGTTGAG GGCTAGCTACAACGA AACTCTTT | 7480 |
| 7674 | GUUGCUCA A CGCGUUGA | 2684 | TCAACGCG GGCTAGCTACAACGA TGAGCAAC | 7481 |
| 7672 | UGCUCAAC G CGUUGAUG | 2685 | CATCAACG GGCTAGCTACAACGA GTTGAGCA | 7482 |
| 7670 | CUCAACGC G UUGAUGGG | 2686 | CCCATCAA GGCTAGCTACAACGA GCGTTGAG | 7483 |
| 7666 | ACGCGUUG A UGGGCAAC | 2687 | GTTGCCCA GGCTAGCTACAACGA CAACGCGT | 7484 |
| 7662 | GUUGAUGG G CAACUUGC | 2688 | GCAAGTTG GGCTAGCTACAACGA CCATCAAC | 7485 |
| 7659 | GAUGGGCA A CUUGCUUU | 2689 | AAAGCAAG GGCTAGCTACAACGA TGCCCATC | 7486 |
| 7655 | GGCAACUU G CUUUCCUC | 2690 | GAGGAAAG GGCTAGCTACAACGA AAGTTGCC | 7487 |
| 7645 | UUUCCUCC G CAGCGCAU | 2691 | ATGCGCTG GGCTAGCTACAACGA GGAGGAAA | 7488 |
| 7642 | CCUCCGCA G CGCAUGGC | 2692 | GCCATGCG GGCTAGCTACAACGA TGCGGAGG | 7489 |
| 7640 | UCCGCAGC G CAUGGCGU | 2693 | ACGCCATG GGCTAGCTACAACGA GCTGCGGA | 7490 |
| 7638 | CGCAGCGC A UGGCGUGA | 2694 | TCACGCCA GGCTAGCTACAACGA GCGCTGCG | 7491 |
| 7635 | AGCGCAUG G CGUGAUCA | 2695 | TGATCACG GGCTAGCTACAACGA CATGCGCT | 7492 |
| 7633 | CGCAUGGC G UGAUCAGG | 2696 | CCTGATCA GGCTAGCTACAACGA GCCATGCG | 7493 |
| 7630 | AUGGCGUG A UCAGGGCG | 2697 | CGCCCTGA GGCTAGCTACAACGA CACGCCAT | 7494 |
| 7624 | UGAUCAGG G CGCCCGUC | 2698 | GACGGGCG GGCTAGCTACAACGA CCTGATCA | 7495 |
| 7622 | AUCAGGGC G CCCGUCCA | 2699 | TGGACGGG GGCTAGCTACAACGA GCCCTGAT | 7496 |
| 7618 | GGGCGCCC G UCCAUGUG | 2700 | CACATGGA GGCTAGCTACAACGA GGGCGCCC | 7497 |
| 7614 | GCCCGUCC A UGUGUAGG | 2701 | CCTACACA GGCTAGCTACAACGA GGACGGGC | 7498 |
| 7612 | CCGUCCAU G UGUAGGAC | 2702 | GTCCTACA GGCTAGCTACAACGA ATGGACGG | 7499 |
| 7610 | GUCCAUGU G UAGGACAU | 2703 | ATGTCCTA GGCTAGCTACAACGA ACATGGAC | 7500 |
| 7605 | UGUGUAGG A CAUCGAGC | 2704 | GCTCGATG GGCTAGCTACAACGA CCTACACA | 7501 |
| 7603 | UGUAGGAC A UCGAGCAG | 2705 | CTGCTCGA GGCTAGCTACAACGA GTCCTACA | 7502 |
| 7598 | GACAUCGA G CAGCAGAC | 2706 | GTCTGCTG GGCTAGCTACAACGA TCGATGTC | 7503 |
| 7595 | AUCGAGCA G CAGACGAC | 2707 | GTCGTCTG GGCTAGCTACAACGA TGCTCGAT | 7504 |
| 7591 | AGCAGCAG A CGACAUCC | 2708 | GGATGTCG GGCTAGCTACAACGA CTGCTGCT | 7505 |
| 7588 | AGCAGACG A CAUCCUCG | 2709 | CGAGGATG GGCTAGCTACAACGA CGTCTGCT | 7506 |
| 7586 | CAGACGAC A UCCUCGCC | 2710 | GGCGAGGA GGCTAGCTACAACGA GTCGTCTG | 7507 |
| 7580 | ACAUCCUC G CCAGCCUC | 2711 | GAGGCTGG GGCTAGCTACAACGA GAGGATGT | 7508 |
| 7576 | CCUCGCCA G CCUCUUCG | 2712 | CGAAGAGG GGCTAGCTACAACGA TGGCGAGG | 7509 |
| 7568 | GCCUCUUC G CUCACGGU | 2713 | ACCGTGAG GGCTAGCTACAACGA GAAGAGGC | 7510 |
| 7564 | CUUCGCUC A CGGUAGAC | 2714 | GTCTACCG GGCTAGCTACAACGA GAGCGAAG | 7511 |
| 7561 | CGCUCACG G UAGACCAA | 2715 | TTGGTCTA GGCTAGCTACAACGA CGTGAGCG | 7512 |
| 7557 | CACGGUAG A CCAAGACC | 2716 | GGTCTTGG GGCTAGCTACAACGA CTACCGTG | 7513 |
| 7551 | AGACCAAG A CCCGUCGC | 2717 | GCGACGGG GGCTAGCTACAACGA CTTGGTCT | 7514 |
| 7547 | GACCCGUC G CUGAGAUC | 2718 | CTCAGCGA GGCTAGCTACAACGA GGGTCTTG | 7515 |
| 7544 | GACCCGUC G CUGAGAUC | 2719 | GATCTCAG GGCTAGCTACAACGA GACGGGTC | 7516 |
| 7538 | UCGCUGAG A UCGGGAUC | 2720 | GATCCCGA GGCTAGCTACAACGA CTCAGCGA | 7517 |
| 7532 | AGAUCGGG A UCCCCCGG | 2721 | CCGGGGGA GGCTAGCTACAACGA CCCGATCT | 7518 |
| 7524 | AUCCCCCG G CUCCCCCU | 2722 | AGGGGGAG GGCTAGCTACAACGA CGGGGGAT | 7519 |
| 7506 | AAGGGGGG G CAUAGAGG | 2723 | CCTCTATG GGCTAGCTACAACGA CCCCCCTT | 7520 |
| 7504 | GGGGGGGC A UAGAGGAG | 2724 | CTCCTCTA GGCTAGCTACAACGA GCCCCCCC | 7521 |
| 7496 | AUAGAGGA G UACGACUC | 2725 | GAGTCGTA GGCTAGCTACAACGA TCCTCTAT | 7522 |
| 7494 | AGAGGAGU A CGACUCAA | 2726 | TTGAGTCG GGCTAGCTACAACGA ACTCCTCT | 7523 |
| 7491 | GGAGUACG A CUCAACGU | 2727 | ACGTTGAG GGCTAGCTACAACGA CGTACTCC | 7524 |
| 7486 | ACGACUCA A CGUCGGAU | 2728 | ATCCGACG GGCTAGCTACAACGA TGAGTCGT | 7525 |
| 7484 | GACUCAAC G UCGGAUCC | 2729 | GGATCCGA GGCTAGCTACAACGA GTTGAGTC | 7526 |
| 7479 | AACGUCGG A UCCUGCGU | 2730 | ACGCAGGA GGCTAGCTACAACGA CCGACGTT | 7527 |
| 7474 | CGGAUCCU G CGUCACCG | 2731 | CGGTGACG GGCTAGCTACAACGA AGGATCCG | 7528 |
| 7472 | GAUCCUGC G UCACCGUC | 2732 | GACGGTGA GGCTAGCTACAACGA GCAGGATC | 7529 |
| 7469 | CCUGCGUC A CCGUCAUU | 2733 | AATGACGG GGCTAGCTACAACGA GACGCAGG | 7530 |
| 7466 | GCGUCACC G UCAUUGGA | 2734 | TCCAATGA GGCTAGCTACAACGA GGTGACGC | 7531 |
| 7463 | UCACCGUC A UUGGAGGU | 2735 | ACCTCCAA GGCTAGCTACAACGA GACGGTGA | 7532 |
| 7456 | CAUUGGAG G UCUGGUCG | 2736 | CGACCAGA GGCTAGCTACAACGA CTCCAATG | 7533 |
| 7451 | GAGGUCUG G UCGGGGGG | 2737 | CCCCCCCA GGCTAGCTACAACGA GGTGACGC | 7534 |
| 7441 | CGGGGGGG G CGGUUGCC | 2738 | GGCAACCG GGCTAGCTACAACGA CCCCCCCG | 7535 |
| 7438 | GGGGGGCG G UUGCCGUA | 2739 | TACGGCAA GGCTAGCTACAACGA CGCCCCCC | 7536 |
| 7435 | GGGCGGUU G CCGUACCU | 2740 | AGGTACGG GGCTAGCTACAACGA AACCGCCC | 7537 |
| 7432 | CGGUUGCC G UACCUCUA | 2741 | TAGAGGTA GGCTAGCTACAACGA GGCAACCG | 7538 |
| 7430 | GUUGCCGU A CCUCUAUC | 2742 | GATAGAGG GGCTAGCTACAACGA ACGGCAAC | 7539 |
| 7424 | GUACCUCU A UCAGCGGC | 2743 | GCCGCTGA GGCTAGCTACAACGA AGAGGTAC | 7540 |
| 7420 | CUCUAUCA G CGGCCGAU | 2744 | ATCGGCCG GGCTAGCTACAACGA TGATAGAG | 7541 |
| 7417 | UAUCAGCG G CCGAUGAU | 2745 | ATCATCGG GGCTAGCTACAACGA CGCTGATA | 7542 |
| 7413 | AGCGGCCG A UGAUUCAG | 2746 | CTGAATCA GGCTAGCTACAACGA CGGCCGCT | 7543 |
| 7410 | GGCCGAUG A UUCAGAGC | 2747 | GCTCTGAA GGCTAGCTACAACGA CATCGGCC | 7544 |
| 7403 | GAUUCAGA G CUGCCGAA | 2748 | TTCGGCAG GGCTAGCTACAACGA TCTGAATC | 7545 |
| 7400 | UCAGAGCU G CCGAAGGU | 2749 | ACCTTCGG GGCTAGCTACAACGA AGCTCTGA | 7546 |
| 7393 | UGCCGAAG G UCUUUGUG | 2750 | CACAAAGA GGCTAGCTACAACGA CTTCGGCA | 7547 |
| 7387 | AGGUCUUU G UGGCGAGC | 2751 | GCTCGCCA GGCTAGCTACAACGA AAAGACCT | 7548 |
| 7384 | UCUUUGUG G CGAGCUCC | 2752 | GGAGCTCG GGCTAGCTACAACGA CACAAAGA | 7549 |
| 7380 | UGUGGCGA G CUCCGCCA | 2753 | CCACCGTG GGCTAGCTACAACGA CTTCTGCC | 7550 |
| 7375 | CGAGCUCC G CCAAGGCA | 2754 | TGCCTTGG GGCTAGCTACAACGA GGAGCTCG | 7551 |
| 7369 | CCGCCAAG G CAGAAGAC | 2755 | GTCTTCTG GGCTAGCTACAACGA CTTGGCGG | 7552 |
| 7362 | GGCAGAAG A CACGGUGG | 2756 | CCACCGTG GGCTAGCTACAACGA CTTCTGCC | 7553 |
| 7360 | CAGAAGAC A CGGUGGAC | 2757 | GTCCACCG GGCTAGCTACAACGA GTCTTCTG | 7554 |
| 7357 | AAGACACG G UGGACUCU | 2758 | AGAGTCCA GGCTAGCTACAACGA CGTGTCTT | 7555 |
| 7353 | CACGGUGG A CUCUGUCA | 2759 | TGACAGAG GGCTAGCTACAACGA CCACCGTG | 7556 |
| 7348 | UGGACUCU G UCAGAACA | 2760 | TGTTCTGA GGCTAGCTACAACGA AGAGTCCA | 7557 |
| 7342 | CUGUCAGA A CAACCGUC | 2761 | GACGGTTG GGCTAGCTACAACGA TCTGACAG | 7558 |
| 7339 | UCAGAACA A CCGUCCUC | 2762 | GAGGACGG GGCTAGCTACAACGA TGTTCTGA | 7559 |
| 7336 | GAACAACC G UCCUCUUC | 2763 | GAAGAGGA GGCTAGCTACAACGA GGTTGTTC | 7560 |
| 7323 | CUUCCUCC G UGGAGGUG | 2764 | CACCTCCA GGCTAGCTACAACGA GGAGGAGG | 7561 |
| 7317 | CCGUGGAG G UGGUAUUG | 2765 | CAATACCA GGCTAGCTACAACGA CTCCACGG | 7562 |
| 7314 | UGGAGGUG G UAUUGGAG | 2766 | CTCCAATA GGCTAGCTACAACGA CACCTCCA | 7563 |
| 7312 | GAGGUGGU A UUGGAGGG | 2767 | CCCTCCAA GGCTAGCTACAACGA ACCACCTC | 7564 |
| 7303 | UUGGAGGG G CCUUGGCA | 2768 | TGCCAAGG GGCTAGCTACAACGA CCCTCCAA | 7565 |
| 7297 | GGGCCUUG G CAGGUGGC | 2769 | GCCACCTG GGCTAGCTACAACGA CAAGGCCC | 7566 |
| 7293 | CUUGGCAG G UGGCAAUG | 2770 | CATTGCCA GGCTAGCTACAACGA CTGCCAAG | 7567 |
| 7290 | GGCAGGUG G CAAUGGGC | 2771 | GCCCATTG GGCTAGCTACAACGA CACCTGCC | 7568 |
| 7287 | AGGUGGCA A UGGGCACC | 2772 | GGTGCCCA GGCTAGCTACAACGA TGCCACCT | 7569 |
| 7283 | GGCAAUGG G CACCCGUG | 2773 | CACGGGTG GGCTAGCTACAACGA CCATTGCC | 7570 |
| 7281 | CAAUGGGC A CCCGUGUA | 2774 | TACACGGG GGCTAGCTACAACGA GCCCATTG | 7571 |
| 7277 | GGGCACCC G UGUACCAC | 2775 | GTGGTACA GGCTAGCTACAACGA GGGTGCCC | 7572 |
| 7275 | GCACCCGU G UACCACCG | 2776 | CGGTGGTA GGCTAGCTACAACGA ACGGGTGC | 7573 |
| 7273 | ACCCGUGU A CCACCGGA | 2777 | TCCGGTGG GGCTAGCTACAACGA ACACGGGT | 7574 |
| 7270 | CGUGUACC A CCGGAGGG | 2778 | CCCTCCGG GGCTAGCTACAACGA GGTACACG | 7575 |
| 7261 | CCGGAGGG A CGUAGUCU | 2779 | AGACTACG GGCTAGCTACAACGA CCCTCCGG | 7576 |
| 7259 | GGAGGGAC G UAGUCUGG | 2780 | CCAGACTA GGCTAGCTACAACGA GTCCCTCC | 7577 |
| 7256 | GGGACGUA G UCUGGGUC | 2781 | GACCCAGA GGCTAGCTACAACGA TACGTCCC | 7578 |
| 7250 | UAGUCUGG G UCUUUCCA | 2782 | TGGAAAGA GGCTAGCTACAACGA CCAGACTA | 7579 |
| 7239 | UUUCCAGG G CUCUAGUA | 2783 | TACTAGAG GGCTAGCTACAACGA CCTGGAAA | 7580 |
| 7233 | GGGCUCUA G UAGUGGAG | 2784 | CTCCACTA GGCTAGCTACAACGA TAGAGCCC | 7581 |
| 7230 | CUCUAGUA G UGGAGGGU | 2785 | ACCCTCCA GGCTAGCTACAACGA TACTAGAG | 7582 |
| 7223 | AGUGGAGG G UUGUAAUC | 2786 | GATTACAA GGCTAGCTACAACGA CCTCCACT | 7583 |
| 7220 | GGAGGGUU G UAAUCCGG | 2787 | CCGGATTA GGCTAGCTACAACGA AACCCTCC | 7584 |
| 7217 | GGGUUGUA A UCCGGGCG | 2788 | CGCCCGGA GGCTAGCTACAACGA TACAACCC | 7585 |
| 7211 | UAAUCCGG G CGUGCCCA | 2789 | TGGGCACG GGCTAGCTACAACGA CCGGATTA | 7586 |
| 7209 | AUCCGGGC G UGCCCAUA | 2790 | TATGGGCA GGCTAGCTACAACGA GCCCGGAT | 7587 |
| 7207 | CCGGGCGU G CCCAUAUG | 2791 | CATATGGG GGCTAGCTACAACGA ACGCCCGG | 7588 |
| 7203 | GCGUGCCC A UAUGGGUA | 2792 | TACCCATA GGCTAGCTACAACGA GGGCACGC | 7589 |
| 7201 | GUGCCCAU A UGGGUAAC | 2793 | GTTACCCA GGCTAGCTACAACGA ATGGGCAC | 7590 |
| 7197 | CCAUAUGG G UAACGCUG | 2794 | CAGCGTTA GGCTAGCTACAACGA CCATATGG | 7591 |
| 7194 | UAUGGGUA A CGCUGAAG | 2795 | CTTCAGCG GGCTAGCTACAACGA TACCCATA | 7592 |
| 7192 | UGGGUAAC G CUGAAGGA | 2796 | TCCTTCAG GGCTAGCTACAACGA GTTACCCA | 7593 |
| 7182 | UGAAGGAA A CUUCUUGG | 2797 | CCAAGAAG GGCTAGCTACAACGA TTCCTTCA | 7594 |
| 7173 | CUUCUUGG A UUUCCGCA | 2798 | TGCGGAAA GGCTAGCTACAACGA CCAAGAAG | 7595 |
| 7167 | GGAUUUCC G CAGGAUCU | 2799 | AGATCCTG GGCTAGCTACAACGA GGAAATCC | 7596 |
| 7162 | UCCGCAGG A UCUCCGCC | 2800 | GGCGGAGA GGCTAGCTACAACGA CCTGCGGA | 7597 |
| 7156 | GGAUCUCC G CCGGAAUG | 2801 | CATTCCGG GGCTAGCTACAACGA GGAGATCC | 7598 |
| 7150 | CCGCCGGA A UGGACACC | 2802 | GGTGTCCA GGCTAGCTACAACGA TCCGGCGG | 7599 |
| 7146 | CGGAAUGG A CACCUCUC | 2803 | GAGAGGTG GGCTAGCTACAACGA CCATTCCG | 7600 |
| 7144 | GAAUGGAC A CCUCUCUC | 2804 | GAGAGAGG GGCTAGCTACAACGA GTCCATTC | 7601 |
| 7133 | UCUCUCUC A UCCUCCUC | 2805 | GAGGAGGA GGCTAGCTACAACGA GAGAGAGA | 7602 |
| 7123 | CCUCCUCC G CUCGAAGC | 2806 | GCTTCGAG GGCTAGCTACAACGA GGAGGAGG | 7603 |
| 7116 | CGCUCGAA G CGGGUCAA | 2807 | TTGACCCG GGCTAGCTACAACGA TTCGAGCG | 7604 |
| 7112 | CGAAGCGG G UCAAAAGA | 2808 | TCTTTTGA GGCTAGCTACAACGA CCGCTTCG | 7605 |
| 7103 | UCAAAAGA G UCCAGGGU | 2809 | ACCCTGGA GGCTAGCTACAACGA TCTTTTGA | 7606 |
| 7096 | AGUCCAGG G UAACUACC | 2810 | GGTAGTTA GGCTAGCTACAACGA CCTGGACT | 7607 |
| 7093 | CCAGGGUA A CUACCUUA | 2811 | TAAGGTAG GGCTAGCTACAACGA TACCCTGG | 7608 |
| 7090 | GGGUAACU A CCUUAUUC | 2812 | GAATAAGG GGCTAGCTACAACGA AGTTACCC | 7609 |
| 7085 | ACUACCUU A UUCUCUGA | 2813 | TCAGAGAA GGCTAGCTACAACGA AAGGTAGT | 7610 |
| 7077 | AUUCUCUG A CUCCACGC | 2814 | GCGTGGAG GGCTAGCTACAACGA CAGAGAAT | 7611 |
| 7072 | CUGACUCC A CGCGAGUG | 2815 | CACTCGCG GGCTAGCTACAACGA GGAGTCAG | 7612 |
| 7070 | GACUCCAC G CGAGUGAU | 2816 | ATCACTCG GGCTAGCTACAACGA GTGGAGTC | 7613 |
| 7066 | CCACGCGA G UGAUGUUA | 2817 | TAACATCA GGCTAGCTACAACGA TCGCGTGG | 7614 |
| 7063 | CGCGAGUG A UGUUACCG | 2818 | CGGTAACA GGCTAGCTACAACGA CACTCGCG | 7615 |
| 7061 | CGAGUGAU G UUACCGCC | 2819 | GGCGGTAA GGCTAGCTACAACGA ATCACTCG | 7616 |
| 7058 | GUGAUGUU A CCGCCCAU | 2820 | ATGGGCGG GGCTAGCTACAACGA AACATCAC | 7617 |
| 7055 | AUGUUACC G CCCAUCUC | 2821 | GAGATGGG GGCTAGCTACAACGA GGTAACAT | 7618 |
| 7051 | UACCGCCC A UCUCCUGC | 2822 | GCAGGAGA GGCTAGCTACAACGA GGGCGGTA | 7619 |
| 7044 | CAUCUCCU G CCGCCACA | 2823 | TGTGGCGG GGCTAGCTACAACGA AGGAGATG | 7620 |
| 7041 | CUCCUGCC G CCACAGGA | 2824 | TCCTGTGG GGCTAGCTACAACGA GGCAGGAG | 7621 |
| 7038 | CUGCCGCC A CAGGAGGU | 2825 | ACCTCCTG GGCTAGCTACAACGA GGCGGCAG | 7622 |
| 7031 | CACAGGAG G UUGGCCUC | 2826 | GAGGCCAA GGCTAGCTACAACGA CTCCTGTG | 7623 |
| 7027 | GGAGGUUG G CCUCGAUG | 2827 | CATCGAGG GGCTAGCTACAACGA CAACCTCC | 7624 |
| 7021 | UGGCCUCG A UGAGGUCA | 2828 | TGACCTCA GGCTAGCTACAACGA CGAGGCCA | 7625 |
| 7016 | UCGAUGAG G UCAAAGUC | 2829 | GACTTTGA GGCTAGCTACAACGA CTCATCGA | 7626 |
| 7010 | AGGUCAAA G UCUGGGGA | 2830 | TCCCCAGA GGCTAGCTACAACGA TTTGACCT | 7627 |
| 7001 | UCUGGGGA G UCAUAUUG | 2831 | CAATATGA GGCTAGCTACAACGA TCCCCAGA | 7628 |
| 6998 | GGGGAGUC A UAUUGGGU | 2832 | ACCCAATA GGCTAGCTACAACGA GACTCCCC | 7629 |
| 6996 | GGAGUCAU A UUGGGUAA | 2833 | TTACCCAA GGCTAGCTACAACGA ATGACTCC | 7630 |
| 6991 | CAUAUUGG G UAAUGUAU | 2834 | ATACATTA GGCTAGCTACAACGA CCAATATG | 7631 |
| 6988 | GGGGAGUC A UAUUGGGU | 2835 | GACATACA GGCTAGCTACAACGA TACCCAAT | 7632 |
| 6986 | UGGGUAAU G UAUGUCGC | 2836 | GCGACATA GGCTAGCTACAACGA ATTACCCA | 7633 |
| 6984 | GGUAAUGU A UGUCGCCU | 2837 | AGGCGACA GGCTAGCTACAACGA ACATTACC | 7634 |
| 6982 | UAAUGUAU G UCGCCUUC | 2838 | GAAGGCGA GGCTAGCTACAACGA ATACATTA | 7635 |
| 6979 | UGUAUGUC G CCUUCGAA | 2839 | TTCGAAGG GGCTAGCTACAACGA CACATACA | 7836 |
| 6966 | CGAAGAAG G CGCAGACA | 2840 | TGTCTGCG GGCTAGCTACAACGA CTTCTTCG | 7637 |
| 6964 | AAGAAGGC G CAGACAGC | 2841 | GCTGTCTG GGCTAGCTACAACGA GCCTTCTT | 7638 |
| 6960 | AGGCGCAG A CAGCUGGC | 2842 | GCCAGCTG GGCTAGCTACAACGA CTGCGCCT | 7639 |
| 6957 | CGCAGACA G CUGGCUAG | 2843 | CTAGCCAG GGCTAGCTACAACGA TGTCTGCG | 7640 |
| 6953 | GACAGCUG G CUAGCUGA | 2844 | TCAGCTAG GGCTAGCTACAACGA CAGCTGTC | 7641 |
| 6949 | GCUGGCUA G CUGAGGAG | 2845 | CTCCTCAG GGCTAGCTACAACGA TAGCCAGC | 7642 |
| 6941 | GCUGAGGA G CUGGCCAA | 2846 | TTGGCCAG GGCTAGCTACAACGA TCCTCAGC | 7643 |
| 6937 | AGGAGCUG G CCAAGGAG | 2847 | CTCCTTGG GGCTAGCTACAACGA CAGCTCCT | 7644 |
| 6921 | GGGGGGAG A CCCCCUGG | 2848 | CCAGGGGG GGCTAGCTACAACGA CTCCCCCC | 7645 |
| 6913 | ACCCCCUG G CCAGCCUA | 2849 | TAGGCTGG GGCTAGCTACAACGA CAGGGGGT | 7646 |
| 6909 | CCUGGCCA G CCUACGCU | 2850 | AGCGTAGG GGCTAGCTACAACGA TGGCCAGG | 7647 |
| 6905 | GCCAGCCU A CGCUUAGC | 2851 | GCTAAGCG GGCTAGCTACAACGA AGGCTGGC | 7648 |
| 6903 | CAGCCUAC G CUUAGCCG | 2852 | CGGCTAAG GGCTAGCTACAACGA GTAGGCTG | 7649 |
| 6898 | UACGCUUA G CCGUCUCU | 2853 | AGAGACGG GGCTAGCTACAACGA TAAGCGTA | 7650 |
| 6895 | GCUUAGCC G UCUCUCCU | 2854 | AGGAGAGA GGCTAGCTACAACGA GGCTAAGC | 7651 |
| 6886 | UCUCUCCU G UAAUGUGG | 2855 | CCACATTA GGCTAGCTACAACGA AGGAGAGA | 7652 |
| 6883 | CUCCUGUA A UGUGGGAG | 2856 | CTCCCACA GGCTAGCTACAACGA TACAGGAG | 7653 |
| 6881 | CCUGUAAU G UGGGAGGG | 2857 | CCCTCCCA GGCTAGCTACAACGA ATTACAGG | 7654 |
| 6872 | UGGGAGGG G UCGGUGAG | 2858 | CTCACCGA GGCTAGCTACAACGA CCCTCCCA | 7655 |
| 6868 | AGGGGUCG G UGAGCAUG | 2859 | CATGCTCA GGCTAGCTACAACGA CGACCCCT | 7656 |
| 6864 | GUCGGUGA G CAUGGACG | 2860 | CGTCCATG GGCTAGCTACAACGA TCACCGAC | 7657 |
| 6862 | CGGUGAGC A UGGACGUG | 2861 | CACGTCCA GGCTAGCTACAACGA GCTCACCG | 7658 |
| 6858 | GAGCAUGG A CGUGAGCA | 2862 | TGCTCACG GGCTAGCTACAACGA CCATGCTC | 7659 |
| 6856 | GCAUGGAC G UGAGCACU | 2863 | AGTGCTCA GGCTAGCTACAACGA GTCCATGC | 7660 |
| 6852 | GGACGUGA G CACUGCUA | 2864 | TAGCAGTG GGCTAGCTACAACGA TCACGTCC | 7661 |
| 6850 | ACGUGAGC A CUGCUACA | 2865 | TGTAGCAG GGCTAGCTACAACGA GCTCACGT | 7662 |
| 6847 | UGAGCACU G CUACAUCC | 2866 | GGATGTAG GGCTAGCTACAACGA AGTGCTCA | 7663 |
| 6844 | GCACUGCU A CAUCCGGU | 2867 | ACCGGATG GGCTAGCTACAACGA AGCAGTGC | 7664 |
| 6842 | ACUGCUAC A UCCGGUUC | 2868 | GAACCGGA GGCTAGCTACAACGA GTAGCAGT | 7665 |
| 6837 | UACAUCCG G UUCGGGCU | 2869 | AGCCCGAA GGCTAGCTACAACGA CGGATGTA | 7666 |
| 6831 | CGGUUCGG G CUCGCAUG | 2870 | CATGCGAG GGCTAGCTACAACGA CCGAACCG | 7667 |
| 6827 | UCGGGCUC G CAUGGGAG | 2871 | CTCCCATG GGCTAGCTACAACGA GAGCCCGA | 7668 |
| 6825 | GGGCUCGC A UGGGAGCU | 2872 | AGCTCCCA GGCTAGCTACAACGA GCGAGCCC | 7669 |
| 6819 | GCAUGGGA G CUGUGACC | 2873 | GGTCACAG GGCTAGCTACAACGA TCCCATGC | 7670 |
| 6816 | UGGGAGCU G UGACCCAA | 2874 | TTGGGTCA GGCTAGCTACAACGA AGCTCCCA | 7671 |
| 6813 | GAGCUGUG A CCCAACCA | 2875 | TGGTTGGG GGCTAGCTACAACGA CACAGCTC | 7672 |
| 6808 | GUGACCCA A CCAGGUAU | 2876 | ATACCTGG GGCTAGCTACAACGA TGGGTCAC | 7673 |
| 6803 | CCAACCAG G UAUUGGUU | 2877 | AACCAATA GGCTAGCTACAACGA CTGGTTGG | 7674 |
| 6801 | AACCAGGU A UUGGUUGA | 2878 | TCAACCAA GGCTAGCTACAACGA ACCTGGTT | 7675 |
| 6797 | AGGUAUUG G UUGAGCCC | 2879 | GGGCTCAA GGCTAGCTACAACGA CAATACCT | 7676 |
| 6792 | UUGGUUGA G CCCGACCU | 2880 | AGGTCGGG GGCTAGCTACAACGA TCAACCAA | 7677 |
| 6787 | UGAGCCCG A CCUGGAAU | 2881 | ATTCCAGG GGCTAGCTACAACGA CGGGCTCA | 7678 |
| 6780 | GACCUGGA A UGUGACCU | 2882 | AGGTCACA GGCTAGCTACAACGA TCCAGGTC | 7679 |
| 6778 | CCUGGAAU G UGACCUCC | 2883 | GGAGGTCA GGCTAGCTACAACGA ATTCCAGG | 7680 |
| 6775 | GGAAUGUG A CCUCCUCC | 2884 | GGAGGAGG GGCTAGCTACAACGA CACATTCC | 7681 |
| 6765 | CUCCUCCC G UAGGAGAG | 2885 | CTCTCCTA GGCTAGCTACAACGA GGGAGGAG | 7682 |
| 6756 | UAGGAGAG G UCCACACG | 2886 | CGTGTGGA GGCTAGCTACAACGA CTCTCCTA | 7683 |
| 6752 | AGAGGUCC A CACGCCGG | 2887 | CCGGCGTG GGCTAGCTACAACGA GGACCTCT | 7684 |
| 6750 | AGGUCCAC A CGCCGGAG | 2888 | CTCCGGCG GGCTAGCTACAACGA GTGGACCT | 7685 |
| 6748 | GUCCACAC G CCGGAGCG | 2889 | CGCTCCGG GGCTAGCTACAACGA GTGTGGAC | 7686 |
| 6742 | ACGCCGGA G CGUUUCUG | 2890 | CAGAAACG GGCTAGCTACAACGA TCCGGCGT | 7687 |
| 6740 | GCCGGAGC G UUUCUGUG | 2891 | CACAGAAA GGCTAGCTACAACGA GCTCCGGC | 7688 |
| 6734 | CGCUUUCU G UGCAGGCG | 2892 | CGCCTGCA GGCTAGCTACAACGA AGAAACGC | 7689 |
| 6732 | GUUUCUGU G CAGGCGUA | 2893 | TACGCCTG GGCTAGCTACAACGA ACAGAAAC | 7690 |
| 6728 | CUGUGCAG G CGUACCCC | 2894 | GGGGTACG GGCTAGCTACAACGA CTGCACAG | 7691 |
| 6726 | GUGCAGGC G UACCCCAU | 2895 | ATGGGGTA GGCTAGCTACAACGA GCCTGCAC | 7692 |
| 6724 | GCAGGCGU A CCCCAUCC | 2896 | GGATGGGG GGCTAGCTACAACGA ACGCCTGC | 7693 |
| 6719 | CGUACCCC A UCCACUUC | 2897 | GAAGTGGA GGCTAGCTACAACGA GGGGTACG | 7694 |
| 6715 | CCCCAUCC A CUUCCGUG | 2898 | CACGGAAG GGCTAGCTACAACGA GGATGGGG | 7695 |
| 6709 | CCACUUCC G UGAAGAAU | 2899 | ATTCTTCA GGCTAGCTACAACGA GGAAGTGG | 7696 |
| 6702 | CGUGAAGA A UUCGGGGG | 2900 | CCCCCGAA GGCTAGCTACAACGA CCCCCGAA | 7697 |
| 6693 | UUCGGGGG G CGGAACCU | 2901 | AGGTTCCG GGCTAGCTACAACGA CCCCCGAA | 7698 |
| 6688 | GGGGCGGA A CCUGGCAC | 2902 | GTGCCAGG GGCTAGCTACAACGA TCCGCCCC | 7699 |
| 6683 | GGAACCUG G CACGGGCA | 2903 | TGCCCGTG GGCTAGCTACAACGA CAGGTTCC | 7700 |
| 6681 | AACCUGGC A CGGGCAUU | 2904 | AATGCCCG GGCTAGCTACAACGA GCCAGGTT | 7701 |
| 6677 | UGGCACGG G CAUUUUAC | 2905 | GTAAAATG GGCTAGCTACAACGA CCGTGCCA | 7702 |
| 6675 | GCACGGGC A UUUUACGU | 2906 | ACGTAAAA GGCTAGCTACAACGA GCCCGTGC | 7703 |
| 6670 | GGCAUUUU A CGUUGUCA | 2907 | TGACAACG GGCTAGCTACAACGA AAAATGCC | 7704 |
| 6668 | CAUUUUAC G UUGUCAGU | 2908 | ACTGACAA GGCTAGCTACAACGA GTAAAATG | 7705 |
| 6665 | UUUACGUU G UCAGUGGU | 2909 | ACCACTGA GGCTAGCTACAACGA AACGTAAA | 7706 |
| 6661 | CGUUGUCA G UGGUCAUG | 2910 | CATCACCA GGCTAGCTACAACGA TGACAACG | 7707 |
| 6658 | UGUCAGUG G UCAUGCCC | 2911 | GGGCATGA GGCTAGCTACAACGA CACTGACA | 7708 |
| 6655 | CAGUGGUC A UGCCCGUC | 2912 | GACGGGCA GGCTAGCTACAACGA GACCACTG | 7709 |
| 6653 | GUGGUCAU G CCCGUCAC | 2913 | GTGACGGG GGCTAGCTACAACGA ATGACCAC | 7710 |
| 6649 | UCAUGCCC G UCACGUAG | 2914 | CTACGTGA GGCTAGCTACAACGA GGGCATGA | 7711 |
| 6646 | UGCCCGUC A CGUAGUGG | 2915 | CCACTACG GGCTAGCTACAACGA GACGGGCA | 7712 |
| 6644 | CCCGUCAC G UAGUGGAA | 2916 | TTCCACTA GGCTAGCTACAACGA GTGACGGG | 7713 |
| 6641 | GUCACGUA G UGGAAAUC | 2917 | GATTTCCA GGCTAGCTACAACGA TACGTGAC | 7714 |
| 6635 | UAGUGGAA A UCCCCCAC | 2918 | GTGGGGGA GGCTAGCTACAACGA TTCCACTA | 7715 |
| 6628 | AAUCCCCC A CCCGCGUA | 2919 | TACGCGGG GGCTAGCTACAACGA GGGGGATT | 7716 |
| 6624 | CCCCACCC G CGUAACCU | 2920 | AGGTTACG GGCTAGCTACAACGA GGGTGGGG | 7717 |
| 6622 | CCACCCGC G UAACCUCC | 2921 | GGAGGTTA GGCTAGCTACAACGA GCGGGTGG | 7718 |
| 6619 | CCCGCGUA A CCUCCACG | 2922 | CGTGGAGG GGCTAGCTACAACGA TACGCGGG | 7719 |
| 6613 | UAACCUCC A CGUACUCC | 2923 | GGAGTACG GGCTAGCTACAACGA GGAGGTTA | 7720 |
| 6611 | ACCUCCAC G UACUCCUC | 2924 | GAGGAGTA GGCTAGCTACAACGA GTGGAGGT | 7721 |
| 6609 | CUCCACGU A CUCCUCAG | 2925 | CTGAGGAG GGCTAGCTACAACGA ACGTGGAG | 7722 |
| 6601 | ACUCCUCA G CGGCCACC | 2926 | GGTGGCCG GGCTAGCTACAACGA TGAGGAGT | 7723 |
| 6598 | CCUCAGCG G CCACCCGC | 2927 | GCGGGTGG GGCTAGCTACAACGA CGCTGAGG | 7724 |
| 6595 | CAGCGGCC A CCCGCCAU | 2928 | ATGGCGGG GGCTAGCTACAACGA GGCCGCTG | 7725 |
| 6591 | GGCCACCC G CCAUAGCG | 2929 | CGCTATGG GGCTAGCTACAACGA GGGTGGCC | 7726 |
| 6588 | CACCCGCC A UAGCGCCC | 2930 | GGGCGCTA GGCTAGCTACAACGA GGCGGGTG | 7727 |
| 6585 | CCGCCAUA G CGCCCAUG | 2931 | CTAGGGCG GGCTAGCTACAACGA TATGGCGG | 7728 |
| 6583 | GCCAUAGC G CCCUAGAA | 2932 | TTCTAGGG GGCTAGCTACAACGA GCTATGGC | 7729 |
| 6575 | GCCCUAGA A UAGUUUGG | 2933 | CCAAACTA GGCTAGCTACAACGA TCTAGGGC | 7730 |
| 6572 | CUAGAAUA G UUUGGCGC | 2934 | GCGCCAAA GGCTAGCTACAACGA TATTCTAG | 7731 |
| 6567 | AUAGUUUG G CGCCGGGG | 2935 | CCCCGGCG GGCTAGCTACAACGA CAAACTAT | 7732 |
| 6565 | AGUUUGGC G CCGGGGAG | 2936 | CTCCCCGG GGCTAGCTACAACGA GCCAAACT | 7733 |
| 6555 | CGGGGAGG G UGUGCAGG | 2937 | CTCCCCGG GGCTAGCTACAACGA GCCAAACT | 7734 |
| 6553 | GGGAGGGU G UGCAGGGG | 2938 | CCCCTGCA GGCTAGCTACAACGA ACCCTCCC | 7735 |
| 6551 | GAGGGUGU G CAGGGGCC | 2939 | GGCCCCTG GGCTAGCTACAACGA ACACCCTC | 7736 |
| 6545 | GUGCAGGG G CCCGUGGU | 2940 | ACCACGGG GGCTAGCTACAACGA CCCTGCAC | 7737 |
| 6541 | AGGGGCCC G UGGUGUAU | 2941 | ATACACCA GGCTAGCTACAACGA GGGCCCCT | 7738 |
| 6538 | GGCCCGUG G UGUAUGCG | 2942 | CGCATACA GGCTAGCTACAACGA CACGGGCC | 7739 |
| 6536 | CCCGUGGU G UAUGCGUU | 2943 | AACGCATA GGCTAGCTACAACGA ACCACGGG | 7740 |
| 6534 | CGUGGUGU A UGCGUUGA | 2944 | TCAACGCA GGCTAGCTACAACGA ACACCACG | 7741 |
| 6532 | UGGUGUAU G CGUUGAUG | 2945 | CATCAACG GGCTAGCTACAACGA ATACACCA | 7742 |
| 6530 | GUGUAUGC G UUGAUGGG | 2946 | CCCATCAA GGCTAGCTACAACGA GCATACAC | 7743 |
| 6526 | AUGCGUUG A UGGGGAAU | 2947 | ATTCCCCA GGCTAGCTACAACGA CAACGCAT | 7744 |
| 6519 | GAUGGGGA A UGUUCCAU | 2948 | ATGGAACA GGCTAGCTACAACGA TCCCCATC | 7745 |
| 6517 | UGGGGAAU G UUCCAUGC | 2949 | GCATGGAA GGCTAGCTACAACGA ATTCCCCA | 7746 |
| 6512 | AAUGUUCC A UGCCACGU | 2950 | ACGTGGCA GGCTAGCTACAACGA GGAACATT | 7747 |
| 6510 | UGUUCCAU G CCACGUGU | 2951 | ACACGTGG GGCTAGCTACAACGA ATGGAACA | 7748 |
| 6507 | UCCAUGCC A CGUGUUGC | 2952 | GCAACACG GGCTAGCTACAACGA GGCATGGA | 7749 |
| 6505 | CAUGCCAC G UGUUGCUA | 2953 | TAGCAACA GGCTAGCTACAACGA GTGGCATG | 7750 |
| 6503 | UGCCACGU G UUGCUACA | 2954 | TGTAGCAA GGCTAGCTACAACGA ACGTGGCA | 7751 |
| 6500 | CACGUGUU G CUACAGGU | 2955 | ACCTGTAG GGCTAGCTACAACGA AACACGTG | 7752 |
| 6497 | GUGUUGCU A CAGGUCUU | 2956 | AAGACCTG GGCTAGCTACAACGA AGCAACAC | 7753 |
| 6493 | UGCUACAG G UCUUAGGC | 2957 | GCCTAAGA GGCTAGCTACAACGA CTGTAGCA | 7754 |
| 6486 | GGUCUUAG G CCCGACGA | 2958 | TCGTCGGG GGCTAGCTACAACGA CTAAGACC | 7755 |
| 6481 | UAGGCCCG A CGAUCCUC | 2959 | GAGGATCG GGCTAGCTACAACGA CGGGCCTA | 7756 |
| 6478 | GCCCGACG A UCCUCAUG | 2960 | CATGAGGA GGCTAGCTACAACGA CGTCGGGC | 7757 |
| 6472 | CGAUCCUC A UGGAACCG | 2961 | CGGTTCCA GGCTAGCTACAACGA GAGGATCG | 7758 |
| 6467 | CUCAUGGA A CCGUUCUU | 2962 | AAGAACGG GGCTAGCTACAACGA TCCATGAG | 7759 |
| 6464 | AUGGAACC G UUCUUGAC | 2963 | GTCAAGAA GGCTAGCTACAACGA GGTTCCAT | 7760 |
| 6457 | CGUUCUUG A CAUGUCCA | 2964 | TGGACATG GGCTAGCTACAACGA CAAGAACG | 7761 |
| 6455 | UUCUUGAC A UGUCCAGU | 2965 | ACTGGACA GGCTAGCTACAACGA GTCAAGAA | 7762 |
| 6453 | CUUGACAU G UCCAGUGA | 2966 | TCACTGGA GGCTAGCTACAACGA ATGTCAAG | 7763 |
| 6448 | CAUGUCCA G UGAUCUGC | 2967 | GCAGATCA GGCTAGCTACAACGA TGGACATG | 7764 |
| 6445 | GUCCAGUG A UCUGCGCU | 2968 | AGCGCAGA GGCTAGCTACAACGA CACTGGAC | 7765 |
| 6441 | AGUGAUCU G CGCUCCGC | 2969 | GCGGAGCG GGCTAGCTACAACGA AGATCACT | 7766 |
| 6439 | UGAUCUGC G CUCCGCAU | 2970 | ATGCGGAG GGCTAGCTACAACGA GCAGATCA | 7767 |
| 6434 | UGCGCUCC G CAUGGGCA | 2971 | TGCCCATG GGCTAGCTACAACGA GGAGCGCA | 7768 |
| 6432 | CGCUCCGC A UGGGCAGG | 2972 | CCTGCCCA GGCTAGCTACAACGA GCGGAGCG | 7769 |
| 6428 | CCGCAUGG G CAGGUGGU | 2973 | ACCACCTG GGCTAGCTACAACGA CCATGCGG | 7770 |
| 6424 | AUGGGCAG G UGGUUUGC | 2974 | GCAAACCA GGCTAGCTACAACGA CTGCCCAT | 7771 |
| 6421 | GGCAGGUG G UUUGCAUG | 2975 | CATGCAAA GGCTAGCTACAACGA CACCTGCC | 7772 |
| 6417 | GGUGGUUU G CAUGAUAC | 2976 | GTATCATG GGCTAGCTACAACGA AAACCAAC | 7773 |
| 6415 | UGGUUUGC A UGAUACCG | 2977 | CGGTATCA GGCTAGCTACAACGA GCAAACCA | 7774 |
| 6412 | UUUGCAUG A UACCGUCU | 2978 | AGACGGTA GGCTAGCTACAACGA CATGCAAA | 7775 |
| 6410 | UGCAUGAU A CCGUCUCC | 2979 | GGAGACGG GGCTAGCTACAACGA ATCATGCA | 7776 |
| 6407 | AUGAUACC G UCUCCCCG | 2980 | CGGGGAGA GGCTAGCTACAACGA GGTATCAT | 7777 |
| 6399 | GUCUCCCC G CCAGACCC | 2981 | GGGTCTGG GGCTAGCTACAACGA GGGGAGAC | 7778 |
| 6394 | CCCGCCAG A CCCCCCUG | 2982 | CAGGGGGG GGCTAGCTACAACGA CTGGCGGG | 7779 |
| 6386 | ACCCCCCU G UACCCACG | 2983 | CGTGGGTA GGCTAGCTACAACGA AGGGGGGT | 7780 |
| 6384 | CCCCCUGU A CCCACGUU | 2984 | AACGTGGG GGCTAGCTACAACGA ACAGGGGG | 7781 |
| 6380 | CUGUACCC A CGUUGGCA | 2985 | TGCCAACG GGCTAGCTACAACGA GGGTACAG | 7782 |
| 6378 | GUACCCAC G UUGGCAUG | 2986 | CATGCCAA GGCTAGCTACAACGA GTGGGTAC | 7783 |
| 6374 | CCACGUUG G CAUGAGAA | 2987 | TTCTCATG GGCTAGCTACAACGA CAACGTGG | 7784 |
| 6372 | ACGUUGGC A UGAGAAGA | 2988 | TCTTCTCA GGCTAGCTACAACGA GCCAACGT | 7785 |
| 6358 | AGAAAGGG A CUCCCGGC | 2989 | GCCGGGAG GGCTAGCTACAACGA CCCTTTCT | 7786 |
| 6351 | GACUCCCG G CAACCGCG | 2990 | CGCGGTTG GGCTAGCTACAACGA CGGGAGTC | 7787 |
| 6348 | UCCCGGCA A CCGCGGCA | 2991 | TGCCGCGG GGCTAGCTACAACGA TGCCGGGA | 7788 |
| 6345 | CGGCAACC G CGGCAGGA | 2992 | TCCTGCCG GGCTAGCTACAACGA GGTTGCCG | 7789 |
| 6342 | CAACCGCG G CAGGAGCU | 2993 | AGCTCCTG GGCTAGCTACAACGA CGCGGTTG | 7790 |
| 6336 | CGGCAGGA G CUUGGACU | 2994 | AGTCCAAG GGCTAGCTACAACGA TCCTGCCG | 7791 |
| 6330 | GAGCUUGG A CUGAAGCC | 2995 | GGCTTCAG GGCTAGCTACAACGA CCAAGCTC | 7792 |
| 6324 | GGACUGAA G CCAGGUCU | 2996 | AGACCTGG GGCTAGCTACAACGA TTCAGTCC | 7793 |
| 6319 | GAAGCCAG G UCUUGAAG | 2997 | CTTCAAGA GGCTAGCTACAACGA CTGGCTTC | 7794 |
| 6311 | GUCUUGAA G UCAGUCAA | 2998 | TTGACTGA GGCTAGCTACAACGA TTCAAGAC | 7795 |
| 6307 | UGAAGUCA G UCAACACC | 2999 | GGTGTTGA GGCTAGCTACAACGA TGACTTCA | 7796 |
| 6303 | GUCAGUCA A CACCGUGC | 3000 | GCACGGTG GGCTAGCTACAACGA TGACTGAC | 7797 |
| 6301 | CAGUCAAC A CCGUGCAU | 3001 | ATGCACGG GGCTAGCTACAACGA GTTGACTG | 7798 |
| 6298 | UCAACACC G UGCAUAUC | 3002 | GATATGCA GGCTAGCTACAACGA GGTGTTGA | 7799 |
| 6296 | AACACCGU G CAUAUCCA | 3003 | TGGATATG GGCTAGCTACAACGA ACGGTGTT | 7800 |
| 6294 | CACCGUGC A UAUCCAGU | 3004 | ACTGGATA GGCTAGCTACAACGA GCACGGTG | 7801 |
| 6292 | CCGUGCAU A UCCAGUCC | 3005 | GGACTGGA GGCTAGCTACAACGA ATGCACGG | 7802 |
| 6287 | CAUAUCCA G UCCCAAAC | 3006 | GTTTGGGA GGCTAGCTACAACGA TGGATATG | 7803 |
| 6280 | AGUCCCAA A CAUCCCUU | 3007 | AAGGGATG GGCTAGCTACAACGA TTGGGACT | 7804 |
| 6278 | UCCCAAAC A UCCCUUAG | 3008 | CTAAGGGA GGCTAGCTACAACGA GTTTGGGA | 7805 |
| 6270 | AUCCCUUA G CCACGAGC | 3009 | GCTCGTGG GGCTAGCTACAACGA TAAGGGAT | 7806 |
| 6267 | CCUUAGCC A CGAGCCGG | 3010 | CCGGCTCG GGCTAGCTACAACGA GGCTAAGG | 7807 |
| 6263 | AGCCACGA G CCGGAACA | 3011 | TGTTCCGG GGCTAGCTACAACGA TCGTGGCT | 7808 |
| 6257 | GAGCCGGA A CAUGGCGU | 3012 | ACGCCATG GGCTAGCTACAACGA TCCGGCTC | 7809 |
| 6255 | GCCGGAAC A UGGCGUGG | 3013 | CCACGCCA GGCTAGCTACAACGA GTTCCGGC | 7810 |
| 6252 | GGAACAUG G CGUGGAGC | 3014 | GCTCCAGG GGCTAGCTACAACGA CATGTTCC | 7811 |
| 6250 | AACAUGGC G UGGAGCAG | 3015 | CTGCTCCA GGCTAGCTACAACGA GCCATGTT | 7812 |
| 6245 | GGCGUGGA G CAGUCCUC | 3016 | GAGGACTG GGCTAGCTACAACGA TCCACGCC | 7813 |
| 6242 | GUGGAGCA G UCCUCAUU | 3017 | AATGAGGA GGCTAGCTACAACGA TGCTCCAC | 7814 |
| 6236 | CAGUCCUC A UUGAUCCA | 3018 | TGGATCAA GGCTAGCTACAACGA GAGGACTG | 7815 |
| 6232 | CCUCAUUG A UCCACUGA | 3019 | TCAGTGGA GGCTAGCTACAACGA CAATGAGG | 7816 |
| 6228 | AUUGAUCC A CUGAUGGA | 3020 | TCCATCAG GGCTAGCTACAACGA GGATCAAT | 7817 |
| 6224 | AUCCACUG A UGGAGCCU | 3021 | AGGCTCCA GGCTAGCTACAACGA CAGTGGAT | 7818 |
| 6219 | CUGAUGGA G CCUCCUCA | 3022 | TGAGGAGG GGCTAGCTACAACGA TCCATCAG | 7819 |
| 6210 | CCUCCUCA G CAGCUGAG | 3023 | CTCAGCTG GGCTAGCTACAACGA TGAGGAGG | 7820 |
| 6207 | CCUCAGCA G CUGAGUGA | 3024 | TCACTCAG GGCTAGCTACAACGA TGCTGAGG | 7821 |
| 6202 | GCAGCUGA G UGAUGGUG | 3025 | CACCATCA GGCTAGCTACAACGA TCAGCTGC | 7822 |
| 6199 | GCUGAGUG A UGGUGAGG | 3026 | CCTCACCA GGCTAGCTACAACGA CACTCAGC | 7823 |
| 6196 | GAGUGAUG G UGAGGCUG | 3027 | CAGCCTCA GGCTAGCTACAACGA CATCACTC | 7824 |
| 6191 | AUGGUGAG G CUGGAGAG | 3028 | CTCTCCAG GGCTAGCTACAACGA CTCACCAT | 7825 |
| 6181 | UGGAGAGG A UUUGUGUG | 3029 | CACACAAA GGCTAGCTACAACGA CCTCTCCA | 7826 |
| 6177 | GAGGAUUU G UGUGACGC | 3030 | GCGTCACA GGCTAGCTACAACGA AAATCCTC | 7827 |
| 6175 | GGAUUUGU G UGACGCGC | 3031 | GCGCGTCA GGCTAGCTACAACGA ACAAATCC | 7828 |
| 6172 | UUUGUGUG A CGCGCGCC | 3032 | GGCGCGCG GGCTAGCTACAACGA CACACAAA | 7829 |
| 6170 | UGUGUGAC G CGCGCCGC | 3033 | GCGGCGCG GGCTAGCTACAACGA GTCACACA | 7830 |
| 6168 | UGUGACGC G CGCCGCUG | 3034 | CAGCGGCG GGCTAGCTACAACGA GCGTCACA | 7831 |
| 6166 | UGACGCGC G CCGCUGCG | 3035 | CGCAGCGG GGCTAGCTACAACGA GCGCGTCA | 7832 |
| 6163 | CGCGCGCC G CUGCGUCG | 3036 | CGACGCAG GGCTAGCTACAACGA GGCGCGCG | 7833 |
| 6160 | GCGCCGCU G CGUCGCUC | 3037 | GAGCGACG GGCTAGCTACAACGA AGCGGCGC | 7834 |
| 6158 | GCCGCUGC G UCGCUCUC | 3038 | GAGAGCGA GGCTAGCTACAACGA GCAGCGGC | 7835 |
| 6155 | GCUGCGUC G CUCUCAGG | 3039 | CCTGAGAG GGCTAGCTACAACGA GACGCAGC | 7836 |
| 6147 | GCUCUCAG G CACAUAGU | 3040 | ACTATGTG GGCTAGCTACAACGA CTGAGAGC | 7837 |
| 6145 | UCUCAGGC A CAUAGUGC | 3041 | GCACTATG GGCTAGCTACAACGA GCCTGAGA | 7838 |
| 6143 | UCAGGCAC A UAGUGCGU | 3042 | ACGCACTA GGCTAGCTACAACGA GTGCCTGA | 7839 |
| 6140 | GGCACAUA G UGCGUGGG | 3043 | CCCACGCA GGCTAGCTACAACGA TATGTGCC | 7840 |
| 6138 | CACAUAGU G CGUGGGGG | 3044 | CCCCCACG GGCTAGCTACAACGA ACTATGTG | 7841 |
| 6136 | CAUAGUGC G UGGGGGAG | 3045 | CTCCCCCA GGCTAGCTACAACGA GCACTATG | 7842 |
| 6127 | UGGGGGAG A CAUGGUUG | 3046 | CAACCATG GGCTAGCTACAACGA CTCCCCCA | 7843 |
| 6125 | GGGGAGAC A UGGUUGCC | 3047 | GGCAACCA GGCTAGCTACAACGA GTCTCCCC | 7844 |
| 6122 | GAGACAUG G UUGCCCCG | 3048 | CGGGGCAA GGCTAGCTACAACGA CATGTCTC | 7845 |
| 6119 | ACAUGGUU G CCCCGCGA | 3049 | TCGCGGGG GGCTAGCTACAACGA AACCATGT | 7846 |
| 6114 | GUUGCCCC G CGAAGCGA | 3050 | TCGCTTCG GGCTAGCTACAACGA GGGGCAAC | 7847 |
| 6109 | CCCGCGAA G CGAACGCU | 3051 | AGCGTTCG GGCTAGCTACAACGA TTCGCGGG | 7848 |
| 6105 | CGAAGCGA A CGCUAUCA | 3052 | TGATAGCG GGCTAGCTACAACGA TCGCTTCG | 7849 |
| 6103 | AAGCGAAC G CUAUCAGC | 3053 | GCTGATAG GGCTAGCTACAACGA GTTCGCTT | 7850 |
| 6100 | CGAACGCU A UCAGCCGA | 3054 | TCGGCTGA GGCTAGCTACAACGA AGCGTTCG | 7851 |
| 6096 | CGCUAUCA G CCGAUUCA | 3055 | TGAATCGG GGCTAGCTACAACGA TGATAGCG | 7852 |
| 6092 | AUCAGCCG A UUCAUCCA | 3056 | TGGATGAA GGCTAGCTACAACGA CGGCTGAT | 7853 |
| 6088 | GCCGAUUC A UCCACUGC | 3057 | GCAGTGGA GGCTAGCTACAACGA GAATCGGC | 7854 |
| 6084 | AUUCAUCC A CUGCACAG | 3058 | CTGTGCAG GGCTAGCTACAACGA GGATGAAT | 7855 |
| 6081 | CAUCCACU G CACAGCGC | 3059 | GCGCTGTG GGCTAGCTACAACGA AGTGGATG | 7856 |
| 6079 | UCCACUGC A CAGCGCCC | 3060 | GGGCGCTG GGCTAGCTACAACGA GCAGTGGA | 7857 |
| 6076 | ACUGCACA G CGCCCUCU | 3061 | AGAGGGCG GGCTAGCTACAACGA TGTGCAGT | 7858 |
| 6074 | UGCACAGC G CCCUCUCC | 3062 | GGAGAGGG GGCTAGCTACAACGA GCTGTGCA | 7859 |
| 6062 | UCUCCUGG G CCCACAUG | 3063 | CATGTGGG GGCTAGCTACAACGA CCAGGAGA | 7860 |
| 6058 | CUGGGCCC A CAUGCCGA | 3064 | TCGGCATG GGCTAGCTACAACGA GGGCCCAG | 7861 |
| 6056 | GGGCCCAC A UGCCGACG | 3065 | CGTCGGCA GGCTAGCTACAACGA GTGGGCCC | 7862 |
| 6054 | GCCCACAU G CCGACGCA | 3066 | TGCGTCGG GGCTAGCTACAACGA ATGTGGGC | 7863 |
| 6050 | ACAUGCCG A CGCAGUAU | 3067 | ATACTGCG GGCTAGCTACAACGA CGGCATGT | 7864 |
| 6048 | AUGCCGAC G CAGUAUCG | 3068 | CGATACTG GGCTAGCTACAACGA GTCGGCAT | 7865 |
| 6045 | CCGACGCA G UAUCGCUG | 3069 | CAGCGATA GGCTAGCTACAACGA TGCGTCGG | 7866 |
| 6043 | GACGCAGU A UCGCUGCG | 3070 | CGCAGCGA GGCTAGCTACAACGA ACTGCGTC | 7867 |
| 6040 | GCAGUAUC G CUGCGCAC | 3071 | GTGCGCAG GGCTAGCTACAACGA GATACTGC | 7868 |
| 6037 | GUAUCGCU G CGCACACC | 3072 | GGTGTGCG GGCTAGCTACAACGA AGCGATAC | 7869 |
| 6035 | AUCGCUGC G CACACCAC | 3073 | GTGGTGTG GGCTAGCTACAACGA GCAGCGAT | 7870 |
| 6033 | CGCUGCGC A CACCACCC | 3074 | GGGTGGTG GGCTAGCTACAACGA GCGCAGCG | 7871 |
| 6031 | CUGCGCAC A CCACCCCG | 3075 | CGGGGTGG GGCTAGCTACAACGA GTGCGCAG | 7872 |
| 6028 | CGCACACC A CCCCGACG | 3076 | CGTCGGGG GGCTAGCTACAACGA GGTGTGCG | 7873 |
| 6022 | CCACCCCG A CGACCAGG | 3077 | CCTGGTCG GGCTAGCTACAACGA CGGGGTGG | 7874 |
| 6019 | CCCCGACG A CCAGGGCG | 3078 | CGCCCTGG GGCTAGCTACAACGA CGTCGGGG | 7875 |
| 6013 | CGACCAGG G CGCCAGGA | 3079 | TCCTGGCG GGCTAGCTACAACGA CCTGGTCG | 7876 |
| 6011 | ACCAGGGC G CCAGGAGA | 3080 | TCTCCTGG GGCTAGCTACAACGA GCCCTGGT | 7877 |
| 5998 | GAGAGAGG A UGGCAGGG | 3081 | CCCTGCCA GGCTAGCTACAACGA CCTCTCTC | 7878 |
| 5995 | AGAGGUAG G CAGGGAGU | 3082 | ACTCCCTG GGCTAGCTACAACGA CATCCTCT | 7879 |
| 5988 | GGCAGGGA G UAAGUUGA | 3083 | TCAACTTA GGCTAGCTACAACGA TCCCTGCC | 7880 |
| 5984 | GGGAGUAA G UUGACCAG | 3084 | CTGGTCAA GGCTAGCTACAACGA TTACTCCC | 7881 |
| 5980 | GUAAGUUG A CCAGGUCC | 3085 | GGACCTGG GGCTAGCTACAACGA CAACTTAC | 7882 |
| 5975 | UUGACCAG G UCCUCGGU | 3086 | ACCGAGGA GGCTAGCTACAACGA CTGGTCAA | 7883 |
| 5968 | GGUCCUCG G UAGAAGGC | 3087 | GCCTTCTA GGCTAGCTACAACGA CGAGGACC | 7884 |
| 5961 | GGUAGAAG G CAUCUCCC | 3088 | GGGAGATG GGCTAGCTACAACGA CTTCTACC | 7885 |
| 5959 | UAGAAGGC A UCUCCCCG | 3089 | CGGGGAGA GGCTAGCTACAACGA GCCTTCTA | 7886 |
| 5951 | AUCUCCCC G CUCAUGAC | 3090 | GTCATGAG GGCTAGCTACAACGA GGGGAGAT | 7887 |
| 5947 | CCCCGCUC A UGACCUUG | 3091 | CAAGGTCA GGCTAGCTACAACGA GAGCGGGG | 7888 |
| 5944 | CGCUCAUG A CCUUGAAG | 3092 | CTTCAAGG GGCTAGCTACAACGA CATGAGCG | 7889 |
| 5935 | CCUUGAAG G CCACGAGA | 3093 | TCTCGTGG GGCTAGCTACAACGA CTTCAAGG | 7890 |
| 5932 | UGAAGGCC A CGAGAGCA | 3094 | TGCTCTCG GGCTAGCTACAACGA GGCCTTCA | 7891 |
| 5926 | CCACGAGA G CACCCGCC | 3095 | GGCGGGTG GGCTAGCTACAACGA TCTCGTGG | 7892 |
| 5924 | ACGAGAGC A CCCGCCAC | 3096 | GTGGCGGG GGCTAGCTACAACGA GCTCTCGT | 7893 |
| 5920 | GAGCACCC G CCACUCCU | 3097 | AGGAGTGG GGCTAGCTACAACGA GGGTGCTC | 7894 |
| 5917 | CACCCGCC A CUCCUGCU | 3098 | AGCAGGAG GGCTAGCTACAACGA GGCGGGTG | 7895 |
| 5911 | CCACUCCU G CUCCAUAG | 3099 | CTATGGAG GGCTAGCTACAACGA AGGAGTGG | 7896 |
| 5906 | CCUGCUCC A UAGCCCGC | 3100 | GCGGGCTA GGCTAGCTACAACGA GGAGCAGG | 7897 |
| 5903 | GCUCCAUA G CCCGCCAG | 3101 | CTGGCGGG GGCTAGCTACAACGA TATGGAGC | 7898 |
| 5899 | CAUAGCCC G CCAGAAUG | 3102 | CATTCTGG GGCTAGCTACAACGA GGGCTATG | 7899 |
| 5893 | CCGCCAGA A UGUCUACA | 3103 | TGTAGACA GGCTAGCTACAACGA TCTGGCGG | 7900 |
| 5891 | GCCAGAAU G UCUACAAG | 3104 | CTTGTAGA GGCTAGCTACAACGA ATTCTGGC | 7901 |
| 5887 | GAAUGUCU A CAAGCACC | 3105 | GGTGCTTG GGCTAGCTACAACGA AGACATTC | 7902 |
| 5883 | GUCUACAA G CACCUUCC | 3106 | GGAAGGTG GGCTAGCTACAACGA TTGTAGAC | 7903 |
| 5881 | CUACAAGC A CCUUCCCA | 3107 | TGGGAAGG GGCTAGCTACAACGA GCTTGTAG | 7904 |
| 5870 | UUCCCAAG G CCUAUGCU | 3108 | AGCATAGG GGCTAGCTACAACGA CTTGGGAA | 7905 |
| 5866 | CAAGGCCU A UGCUGCCA | 3109 | TGGCAGCA GGCTAGCTACAACGA AGGCCTTG | 7906 |
| 5864 | AGGCCUAU G CUGCCAAC | 3110 | GTTGGCAG GGCTAGCTACAACGA ATAGGCCT | 7907 |
| 5861 | CCUAUGCU G CCAACAGC | 3111 | GCTGTTGG GGCTAGCTACAACGA AGCATAGG | 7908 |
| 5857 | UGCUGCCA A CAGCCGCG | 3112 | CGCGGCTG GGCTAGCTACAACGA TGGCAGCA | 7909 |
| 5854 | UGCCAACA G CCGCGCCA | 3113 | TGGCGCGG GGCTAGCTACAACGA TGTTGGCA | 7910 |
| 5851 | CAACAGCC G CGCCAGCG | 3114 | CGCTGGCG GGCTAGCTACAACGA GGCTGTTG | 7911 |
| 5849 | ACAGCCGC G CCAGCGAU | 3115 | ATCGCTGG GGCTAGCTACAACGA GCGGCTGT | 7912 |
| 5845 | CCGCGCCA G CGAUGCCG | 3116 | CGGCATCG GGCTAGCTACAACGA TGGCGCGG | 7913 |
| 5842 | CGCCAGCG A UGCCGGCG | 3117 | CGCCGGCA GGCTAGCTACAACGA CGCTGGCG | 7914 |
| 5840 | CCAGCGAU G CCGGCGCC | 3118 | GGCGCCGG GGCTAGCTACAACGA ATCGCTGG | 7915 |
| 5836 | CGAUGCCG G CGCCCACG | 3119 | CGTGGGCG GGCTAGCTACAACGA CGGCATCG | 7916 |
| 5834 | AUGCCGGC G CCCACGAA | 3120 | TTCGTGGG GGCTAGCTACAACGA GCCGGCAT | 7917 |
| 5830 | CGGCGCCC A CGAAGGCC | 3121 | GGCCTTCG GGCTAGCTACAACGA GGGCGCCG | 7918 |
| 5824 | CCACGAAG G CCGAAACG | 3122 | CGTTTCGG GGCTAGCTACAACGA CTTCGTGG | 7919 |
| 5818 | AGGCCGAA A CGGCUCUG | 3123 | CAGAGCCG GGCTAGCTACAACGA TTCGGCCT | 7920 |
| 5815 | CCGAAACG G CUCUGGGG | 3124 | CCCCAGAG GGCTAGCTACAACGA CGTTTCGG | 7921 |
| 5803 | UGGGGGGA G CGAGUUGG | 3125 | CCAACTCG GGCTAGCTACAACGA TCCCCCCA | 7922 |
| 5799 | GGGAGCGA G UUGGGCGG | 3126 | CCGCCCAA GGCTAGCTACAACGA TCGCTCCC | 7923 |
| 5794 | CGAGUUGG G CGGCCACC | 3127 | GGTGGCCG GGCTAGCTACAACGA CCAACTCG | 7924 |
| 5791 | GUUGGGCG G CCACCCAC | 3128 | GTGGGTGG GGCTAGCTACAACGA CGCCCAAC | 7925 |
| 5788 | GGGCGGCC A CCCACCCU | 3129 | AGGGTGGG GGCTAGCTACAACGA GGCCGCCC | 7926 |
| 5784 | GGCCACCC A CCCUCCCA | 3130 | TGGGAGGG GGCTAGCTACAACGA GGGTGGCC | 7927 |
| 5773 | CUCCCAAG A UGUUGAAC | 3131 | GTTCAACA GGCTAGCTACAACGA CTTGGGAG | 7928 |
| 5771 | CCCAAGAU G UUGAACAG | 3132 | CTGTTCAA GGCTAGCTACAACGA ATCTTGGG | 7929 |
| 5766 | GAUGUUGA A CAGGAGGG | 3133 | CCCTCCTG GGCTAGCTACAACGA TCAACATC | 7930 |
| 5758 | ACAGGAGG G UGCUUUGG | 3134 | CCAAAGCA GGCTAGCTACAACGA CCTCCTGT | 7931 |
| 5756 | AGGAGGGU G CUUUGGGU | 3135 | ACCCAAAG GGCTAGCTACAACGA ACCCTCCT | 7932 |
| 5749 | UGCUUUGG G UGGUGAGC | 3136 | GCTCACCA GGCTAGCTACAACGA CCAAAGCA | 7933 |
| 5746 | UUUGGGUG G UGAGCGGG | 3137 | CCCGCTCA GGCTAGCTACAACGA CACCCAAA | 7934 |
| 5742 | GGUGGUGA G CGGGCUGG | 3138 | CCAGCCCG GGCTAGCTACAACGA TCACCACC | 7935 |
| 5738 | GUGAGCGG G CUGGUGAU | 3139 | ATCACCAG GGCTAGCTACAACGA CCGCTCAC | 7936 |
| 5734 | GCGGGCUG G UGAUGGAG | 3140 | CTCCATCA GGCTAGCTACAACGA CAGCCCGC | 7937 |
| 5731 | GGCUGGUG A UGGAGGCU | 3141 | AGCCTCCA GGCTAGCTACAACGA CACCAGCC | 7938 |
| 5725 | UGAUGGAG G CUGUGAAU | 3142 | ATTCACAG GGCTAGCTACAACGA CTCCATCA | 7939 |
| 5722 | UGGAGGCU G UGAAUGCC | 3143 | GGCATTCA GGCTAGCTACAACGA AGCCTCCA | 7940 |
| 5718 | GGCUGUGA A UGCCAUCA | 3144 | TGATGGCA GGCTAGCTACAACGA TCACAGCC | 7941 |
| 5716 | CUGUGAAU G CCAUCAAU | 3145 | ATTGATGG GGCTAGCTACAACGA ATTCACAG | 7942 |
| 5713 | UGAAUGCC A UCAAUGAU | 3146 | ATCATTGA GGCTAGCTACAACGA GGCATTCA | 7943 |
| 5709 | UGCCAUCA A UGAUGCUA | 3147 | TAGCATCA GGCTAGCTACAACGA TGATGGCA | 7944 |
| 5706 | CAUCAAUG A UGCUAUCG | 3148 | CGATAGCA GGCTAGCTACAACGA CATTGATG | 7945 |
| 5704 | UCAACGAU G CUAUCGCG | 3149 | CGCGATAG GGCTAGCTACAACGA ATCATTGA | 7946 |
| 5701 | AUGAUGCU A UCGCGGGG | 3150 | CCCCGCGA GGCTAGCTACAACGA AGCATCAT | 7947 |
| 5698 | AUGCUAUC G CGGGGUUC | 3151 | GAACCCCG GGCTAGCTACAACGA GATAGCAT | 7948 |
| 5693 | AUCGCGGG G UUCCCAGG | 3152 | CCTGGGAA GGCTAGCTACAACGA CCCGCGAT | 7949 |
| 5685 | GUUCCCAG G CAGAGUGG | 3153 | CCACTCTG GGCTAGCTACAACGA CTGGGAAC | 7950 |
| 5680 | CAGGCAGA G UGGACAAG | 3154 | CTTGTCCA GGCTAGCTACAACGA TCTGCCTG | 7951 |
| 5676 | CAGAGUGG A CAAGCCUG | 3155 | CAGGCTTG GGCTAGCTACAACGA CCACTCTG | 7952 |
| 5672 | GUGGACAA G CCUGCUAG | 3156 | CTAGCAGG GGCTAGCTACAACGA TTGTCCAC | 7953 |
| 5668 | ACAAGCCU G CUAGGUAC | 3157 | GTACCTAG GGCTAGCTACAACGA AGGCTTGT | 7954 |
| 5663 | CCUGCUAG G UACUGUAU | 3158 | ATACAGTA GGCTAGCTACAACGA CTAGCAGG | 7955 |
| 5661 | UGCUAGGU A CUGUAUCC | 3159 | GGATACAG GGCTAGCTACAACGA ACCTAGCA | 7956 |
| 5658 | UAGGUACU G UAUCCCGC | 3160 | GCGGGATA GGCTAGCTACAACGA AGTACCTA | 7957 |
| 5656 | GGUACUGU A UCCCGCUG | 3161 | CAGCGGGA GGCTAGCTACAACGA ACAGTACC | 7958 |
| 5651 | UGUAUCCC G CUGAUGAA | 3162 | TTCATCAG GGCTAGCTACAACGA GGGATACA | 7959 |
| 5647 | UCCCGCUG A UGAAAUUC | 3163 | GAATTTCA GGCTAGCTACAACGA CAGCGGGA | 7960 |
| 5642 | CUGAUGAA A UUCCACAU | 3164 | ATGTGGAA GGCTAGCTACAACGA TTCATCAG | 7961 |
| 5637 | GAAAUUCC A CAUGUGCU | 3165 | AGCACATG GGCTAGCTACAACGA GGAATTTC | 7962 |
| 5635 | AAUUCCAC A UGUGCUUC | 3166 | GAAGCACA GGCTAGCTACAACGA GTGGAATT | 7963 |
| 5633 | UUCCACAU G UGCUUCGC | 3167 | GCGAAGCA GGCTAGCTACAACGA ATGTGGAA | 7964 |
| 5631 | CCACAUGU G CUUCGCCC | 3168 | GGGCGAAG GGCTAGCTACAACGA ACATGTGG | 7965 |
| 5626 | UGUGCUUC G CCCAGAAA | 3169 | TTTCTGGG GGCTAGCTACAACGA GAAGCACA | 7966 |
| 5617 | CCCAGAAA G CCUCAAGG | 3170 | CCTTGAGG GGCTAGCTACAACGA TTTCTGGG | 7967 |
| 5608 | CCUCAAGG G CUCGCCAC | 3171 | GTGGCGAG GGCTAGCTACAACGA CCTTGAGG | 7968 |
| 5604 | AAGGGCUC G CCACUUGG | 3172 | CCAAGTGG GGCTAGCTACAACGA GAGCCCTT | 7969 |
| 5601 | GGCUCGCC A CUUGGAUU | 3173 | AATCCAAG GGCTAGCTACAACGA GGCGAGCC | 7970 |
| 5595 | CCACUUGG A UUCCACCA | 3174 | TGGTGGAA GGCTAGCTACAACGA CCAAGTGG | 7971 |
| 5590 | UGGAUUCC A CCACGGGA | 3175 | TCCCGTGG GGCTAGCTACAACGA GGAATCCA | 7972 |
| 5587 | AUUCCACC A CGGGAGCA | 3176 | TGCTCCCG GGCTAGCTACAACGA GGTGGAAT | 7973 |
| 5581 | CCACGGGA G CAGCAGCC | 3177 | GGCTGCTG GGCTAGCTACAACGA TCCCGTGG | 7974 |
| 5578 | CGGGAGCA G CAGCCUCC | 3178 | GGAGGCTG GGCTAGCTACAACGA TGCTCCCG | 7975 |
| 5575 | GAGCAGCA G CCUCCGCU | 3179 | AGCGGAGG GGCTAGCTACAACGA TGCTGCTC | 7976 |
| 5569 | CAGCCUCC G CUUGGUUG | 3180 | CAACCAAG GGCTAGCTACAACGA GGAGGCTG | 7977 |
| 5564 | UCCGCUUG G UUGGUGGC | 3181 | GCCACCAA GGCTAGCTACAACGA CAAGCGGA | 7978 |
| 5560 | CUUGGUUG G UGGCUGUU | 3182 | AACAGCCA GGCTAGCTACAACGA CAACCAAG | 7979 |
| 5557 | GGUUGGUG G CUGUUUGC | 3183 | GCAAACAG GGCTAGCTACAACGA CACCAACC | 7980 |
| 5554 | UGGUGGCU G UUUGCAGC | 3184 | GCTGCAAA GGCTAGCTACAACGA AGCCACCA | 7981 |
| 5550 | GGCUGUUU G CAGCAAUC | 3185 | GATTGCTG GGCTAGCTACAACGA AAACAGCC | 7982 |
| 5547 | UGUUUGCA G CAAUCCGA | 3186 | TCGGATTG GGCTAGCTACAACGA TGCAAACA | 7983 |
| 5544 | UUGCAGCA A UCCGAGCG | 3187 | CGCTCGGA GGCTAGCTACAACGA TGCTGCAA | 7984 |
| 5538 | CAAUCCGA G CGCCUUCU | 3188 | AGAAGGCG GGCTAGCTACAACGA TCGGATTG | 7985 |
| 5536 | AUCCGAGC G CCUUCUGC | 3189 | GCAGAAGG GGCTAGCTACAACGA GCTCGGAT | 7986 |
| 5529 | CGCCUUCU G CUUGAACU | 3190 | AGTTCAAG GGCTAGCTACAACGA AGAAGGCG | 7987 |
| 5523 | CUGCUUGA A CUGCUCGG | 3191 | CCGAGCAG GGCTAGCTACAACGA TCAAGCAG | 7988 |
| 5520 | CUUGAACU G CUCGGCGA | 3192 | TCGCCGAG GGCTAGCTACAACGA AGTTCAAG | 7989 |
| 5515 | ACUGCUCG G CGAGCUGC | 3193 | GCAGCTCG GGCTAGCTACAACGA CGAGCAGT | 7990 |
| 5511 | CUCGGCGA G CUGCAUCC | 3194 | GGATGCAG GGCTAGCTACAACGA TCGCCGAG | 7991 |
| 5508 | GGCGAGCU G CAUCCCCU | 3195 | AGGGGATG GGCTAGCTACAACGA AGCTCGCC | 7992 |
| 5506 | CGAGCUGC A UCCCCUGU | 3196 | ACAGGGGA GGCTAGCTACAACGA GCAGCTCG | 7993 |
| 5499 | CAUCCCCU G UUCGAUGU | 3197 | ACATCGAA GGCTAGCTACAACGA AGGGGATG | 7994 |
| 5494 | CCUGUUCG A UGUAAGGG | 3198 | CCCTTACA GGCTAGCTACAACGA CGAACAGG | 7995 |
| 5492 | UGUUCGAU G UAAGGGAG | 3199 | CTCCCTTA GGCTAGCTACAACGA ATCGAACA | 7996 |
| 5483 | UAAGGGAG G UGUGAGGC | 3200 | GCCTCACA GGCTAGCTACAACGA CTCCCTTA | 7997 |
| 5481 | AGGGAGGU G UGAGGCAC | 3201 | GTGCCTCA GGCTAGCTACAACGA ACCTCCCT | 7998 |
| 5476 | GGUGUGAG G CACACUCC | 3202 | GGAGTGTG GGCTAGCTACAACGA CTCACACC | 7999 |
| 5474 | UGUGAGGC A CACUCCUC | 3203 | GAGGAGTG GGCTAGCTACAACGA GCCTCACA | 8000 |
| 5472 | UGAGGCAC A CUCCUCCA | 3204 | TGGAGGAG GGCTAGCTACAACGA GTGCCTCA | 8001 |
| 5464 | ACUCCUCC A UCUCAUCG | 3205 | CGATGAGA GGCTAGCTACAACGA GGAGGAGT | 8002 |
| 5459 | UCCAUCUC A UCGAACUC | 3206 | GAGTTCGA GGCTAGCTACAACGA GAGATGGA | 8003 |
| 5454 | CUCAUCGA A CUCCUGGU | 3207 | ACCAGGAG GGCTAGCTACAACGA TCGATGAG | 8004 |
| 5447 | AACUCCUG G UAGAGAGC | 3208 | GCTCTCTA GGCTAGCTACAACGA CAGGAGTT | 8005 |
| 5440 | GGUAGAGA G CCUCCCUG | 3209 | CAGGGAGG GGCTAGCTACAACGA TCTCTACC | 8006 |
| 5432 | GCCUCCCU G UCGGGGAU | 3210 | ATCCCCGA GGCTAGCTACAACGA AGGGAGGC | 8007 |
| 5425 | UGUCGGGG A UAACAGCC | 3211 | GGCTGTTA GGCTAGCTACAACGA CCCCGACA | 8008 |
| 5422 | CGGGGAUA A CAGCCGGC | 3212 | GCCGGCTG GGCTAGCTACAACGA TATCCCCG | 8009 |
| 5419 | GGAUAACA G CCGGCUUC | 3213 | GAAGCCGG GGCTAGCTACAACGA TGTTATCC | 8010 |
| 5415 | AACAGCCG G CUUCCCGG | 3214 | CCGGGAAG GGCTAGCTACAACGA CGGCTGTT | 8011 |
| 5406 | CUUCCCGG A CAAGAUGA | 3215 | TCATCTTG GGCTAGCTACAACGA CCGGGAAG | 8012 |
| 5401 | CGGACAAG A UGAUUCUG | 3216 | CAGAATCA GGCTAGCTACAACGA CTTGTCCG | 8013 |
| 5398 | ACAAGAUG A UUCUGCCC | 3217 | GGGCAGAA GGCTAGCTACAACGA CATCTTGT | 8014 |
| 5393 | AUGAUUCU G CCCACAAU | 3218 | ATTGTGGG GGCTAGCTACAACGA AGAATCAT | 8015 |
| 5389 | UUCUGCCC A CAAUGACC | 3219 | GGTCATTG GGCTAGCTACAACGA GGGCAGAA | 8016 |
| 5386 | UGCCCACA A UGACCACG | 3220 | CGTGGTCA GGCTAGCTACAACGA TGTGGGCA | 8017 |
| 5383 | CCACAAUG A CCACGCUG | 3221 | CAGCGTGG GGCTAGCTACAACGA CATTGTGG | 8018 |
| 5380 | CAAUGACC A CGCUGCCU | 3222 | AGGCAGCG GGCTAGCTACAACGA GGTCATTG | 8019 |
| 5378 | AUGACCAC G CUGCCUGU | 3223 | ACAGGCAG GGCTAGCTACAACGA GTGGTCAT | 8020 |
| 5375 | ACCACGCU G CCUGUCGU | 3224 | ACGACAGG GGCTAGCTACAACGA AGCGTGGT | 8021 |
| 5371 | CGCUGCCU G UCGUCAGG | 3225 | CCTGACGA GGCTAGCTACAACGA AGGCAGCG | 8022 |
| 5368 | UGCCUGUC G UCAGGCAA | 3226 | TTGCCTGA GGCTAGCTACAACGA GACAGGCA | 8023 |
| 5363 | GUCGUCAG G CAAUACGC | 3227 | GCGTATTG GGCTAGCTACAACGA CTGACGAC | 8024 |
| 5360 | GUCAGGCA A UACGCGGU | 3228 | ACCGCGTA GGCTAGCTACAACGA TGCCTGAC | 8025 |
| 5358 | CAGGCAAU A CGCGGUCA | 3229 | TGACCGCG GGCTAGCTACAACGA ATTGCCTG | 8026 |
| 5356 | GGCAAUAC G CGGUCAGA | 3230 | TCTGACCG GGCTAGCTACAACGA GTATTGCC | 8027 |
| 5353 | AAUACGCG G UCAGAGCU | 3231 | AGCTCTGA GGCTAGCTACAACGA CGCGTATT | 8028 |
| 5347 | CGGUCAGA G CUGCCAGG | 3232 | CCTGGCAG GGCTAGCTACAACGA TCTGACCG | 8029 |
| 5344 | UCAGAGCU G CCAGGACG | 3233 | CGTCCTGG GGCTAGCTACAACGA AGCTCTGA | 8030 |
| 5338 | CUGCCAGG A CGCCACCU | 3234 | AGGTGGCG GGCTAGCTACAACGA CCTGGCAG | 8031 |
| 5336 | GCCAGGAC G CCACCUAC | 3235 | GTAGGTGG GGCTAGCTACAACGA GTCCTGGC | 8032 |
| 5333 | AGGACGCC A CCUACUAG | 3236 | CTAGTAGG GGCTAGCTACAACGA GGCGTCCT | 8033 |
| 5329 | CGCCACCU A CUAGCACC | 3237 | GGTGCTAG GGCTAGCTACAACGA AGGTGGCG | 8034 |
| 5325 | ACCUACUA G CACCCAGG | 3238 | CCTGGGTG GGCTAGCTACAACGA TAGTAGGT | 8035 |
| 5323 | CUACUAGC A CCCAGGUG | 3239 | CACCTGGG GGCTAGCTACAACGA GCTAGTAG | 8036 |
| 5317 | GCACCCAG G UGCUGGUG | 3240 | CACCAGCA GGCTAGCTACAACGA CTGGGTGC | 8037 |
| 5315 | ACCCAGGU G CUGGUGAC | 3241 | GTCACCAG GGCTAGCTACAACGA ACCTGGGT | 8038 |
| 5311 | AGGUGCUG G UGACGACC | 3242 | GGTCGTCA GGCTAGCTACAACGA CAGCACCT | 8039 |
| 5308 | UGCUGGUG A CGACCUCC | 3243 | GGAGGTCG GGCTAGCTACAACGA CACCAGCA | 8040 |
| 5305 | UGGUGACG A CCUCCAGG | 3244 | CCTGGAGG GGCTAGCTACAACGA CGTCACCA | 8041 |
| 5297 | ACCUCCAG G UCAGCCGA | 3245 | TCGGCTGA GGCTAGCTACAACGA CTGGAGGT | 8042 |
| 5293 | CCAGGUCA G CCGACAUG | 3246 | CATGTCGG GGCTAGCTACAACGA TGACCTGG | 8043 |
| 5289 | GUCAGCCG A CAUGCAUG | 3247 | CATGCATG GGCTAGCTACAACGA CGGCTGAC | 8044 |
| 5287 | CAGCCGAC A UGCAUGUC | 3248 | GACATGCA GGCTAGCTACAACGA GTCGGCTG | 8045 |
| 5285 | GCCGACAU G CAUGUCAU | 3249 | ATGACATG GGCTAGCTACAACGA ATGTCGGC | 8046 |
| 5283 | CGACAUGC A UGUCAUGA | 3250 | TCATGACA GGCTAGCTACAACGA GCATGTCG | 8047 |
| 5281 | ACAUGCAU G UCAUGAUG | 3251 | CATCATGA GGCTAGCTACAACGA ATGCATGT | 8048 |
| 5278 | UGCAUGUC A UGAUGUAU | 3252 | ATACATCA GGCTAGCTACAACGA GACATGCA | 8049 |
| 5275 | AUGUCAUG A UGUAUUUG | 3253 | CAAATACA GGCTAGCTACAACGA CATCACAT | 8050 |
| 5273 | GUCAUGAU G UAUUUGGU | 3254 | ACCAAATA GGCTAGCTACAACGA ATCATGAC | 8051 |
| 5271 | CAUGAUGU A UUUGGUUA | 3255 | TAACCAAA GGCTAGCTACAACGA ACATCATG | 8052 |
| 5266 | UGUAUUUG G UUAUGGGG | 3256 | CCCCATAA GGCTAGCTACAACGA CAAATACA | 8053 |
| 5263 | AUUUGGUU A UGGGGUGU | 3257 | ACACCCCA GGCTAGCTACAACGA AACCAAAT | 8054 |
| 5258 | GUUAUGGG G UGUGUGAG | 3258 | CTCACACA GGCTAGCTACAACGA CCCATAAC | 8055 |
| 5256 | UAUGGGGU G UGUGAGGG | 3259 | CCCTCACA GGCTAGCTACAACGA ACCCCATA | 8056 |
| 5254 | UGGGGUGU G UGAGGGUG | 3260 | CACCCTCA GGCTAGCTACAACGA ACACCCCA | 8057 |
| 5248 | GUGUGAGG G UGACAUCA | 3261 | TGATGTCA GGCTAGCTACAACGA CCTCACAC | 8058 |
| 5245 | UGAGGGUG A CAUCAUUU | 3262 | AAATGATG GGCTAGCTACAACGA CACCCTCA | 8059 |
| 5243 | AGGGUGAC A UCAUUUUG | 3263 | CAAAATGA GGCTAGCTACAACGA GTCACCCT | 8060 |
| 5240 | GUGACAUC A UUUUGGAC | 3264 | GTCCAAAA GGCTAGCTACAACGA GATGTCAC | 8061 |
| 5233 | CAUUUUGG A CGGCUCCU | 3265 | AGGAGCCG GGCTAGCTACAACGA CCAAAATG | 8062 |
| 5230 | UUUGGACG G CUCCAUGC | 3266 | GCTAGGAG GGCTAGCTACAACGA CGTCCAAA | 8063 |
| 5223 | GGCUCCUA G CCUAUACA | 3267 | TGTATAGG GGCTAGCTACAACGA TAGGAGCC | 8064 |
| 5219 | CCUAGCCU A UACAGCAG | 3268 | CTGCTGTA GGCTAGCTACAACGA AGGCTAGG | 8065 |
| 5217 | UAGCCUAU A CAGCAGGG | 3269 | CCCTGCTG GGCTAGCTACAACGA ATAGGCTA | 8066 |
| 5214 | CCUAUACA G CAGGGGUG | 3270 | CACCCCTG GGCTAGCTACAACGA TGTATAGG | 8067 |
| 5208 | CAGCAGGG G UGUUGGCC | 3271 | GGCCAACA GGCTAGCTACAACGA CCCTGCTG | 8068 |
| 5206 | GCAGGGGU G UUGGCCCG | 3272 | CGGGCCAA GGCTAGCTACAACGA ACCCCTGC | 8069 |
| 5202 | GGGUGUUG G CCCGUGUA | 3273 | TACACGGG GGCTAGCTACAACGA CAACACCC | 8070 |
| 5198 | GUUGGCCC G UGUAGCGU | 3274 | ACGCTACA GGCTAGCTACAACGA GGGCCAAC | 8071 |
| 5196 | UGGCCCGU G UAGCGUAG | 3275 | CTACGCTA GGCTAGCTACAACGA ACGGGCCA | 8072 |
| 5193 | CCCGUGUA G CGUAGGCU | 3276 | AGCCTACG GGCTAGCTACAACGA TACACGGG | 8073 |
| 5191 | CGUGUAGC G UAGGCUUU | 3277 | AAAGCCTA GGCTAGCTACAACGA GCTACACG | 8074 |
| 5187 | UAGCGUAG G CUUUAGCC | 3278 | GGCTAAAG GGCTAGCTACAACGA CTACGCTA | 8075 |
| 5181 | AGGCUUUA G CCGUGUGA | 3279 | TCACACGG GGCTAGCTACAACGA TAAAGCCT | 8076 |
| 5178 | CUUUAGCC G UGUGAGAC | 3280 | GTCTCACA GGCTAGCTACAACGA GGCTAAAG | 8077 |
| 5276 | UUAGCCGU G UGAGACAC | 3281 | GTGTCTCA GGCTAGCTACAACGA ACGGCTAA | 8078 |
| 5171 | CGUGUGAG A CACUUCCA | 3282 | TGGAAGTG GGCTAGCTACAACGA CTCACACG | 8079 |
| 5169 | UGUGAGAC A CUUCCACA | 3283 | TGTGGAAG GGCTAGCTACAACGA GTCTCACA | 8080 |
| 5163 | ACACUUCC A CAUUUGAU | 3284 | ATCAAATG GGCTAGCTACAACGA GGAAGTGT | 8081 |
| 5161 | ACUUCCAC A UUUGAUCC | 3285 | GGATCAAA GGCTAGCTACAACGA GTGGAAGT | 8082 |
| 5156 | CACAUUUG A UCCCACGA | 3286 | TCGTGGGA GGCTAGCTACAACGA CAAATGTG | 8083 |
| 5151 | UUGAUCCC A CGAUGGGG | 3287 | CCCCATCG GGCTAGCTACAACGA GGGATCAA | 8084 |
| 5148 | AUCCCACG A UGGGGGUG | 3288 | CACCCCCA GGCTAGCTACAACGA CGTGGGAT | 8085 |
| 5142 | CGAUGGGG G UGGAGCCU | 3289 | AGGCTCCA GGCTAGCTACAACGA CCCCATCG | 8086 |
| 5137 | GGGGUGGA G CCUGAGCC | 3290 | GGCTCAGG GGCTAGCTACAACGA TCCACCCC | 8087 |
| 5131 | GAGCCUGA G CCCUGGCG | 3291 | CGCCAGGG GGCTAGCTACAACGA TCAGGCTC | 8088 |
| 5125 | GAGCCCUG G CGCACACU | 3292 | AGTGTGCG GGCTAGCTACAACGA CAGGGCTC | 8089 |
| 5123 | GCCCUGGC G CACACUGU | 3293 | ACAGTGTG GGCTAGCTACAACGA GCCAGGGC | 8090 |
| 5121 | CCUGGCGC A CACUGUGG | 3294 | CCACAGTG GGCTAGCTACAACGA GCGCCAAG | 8091 |
| 5119 | UGGCGCAC A CUGUGGCU | 3295 | AGCCACAG GGCTAGCTACAACGA GTGCGCCA | 8092 |
| 5116 | CGCACACU G UGGCUUGG | 3296 | CCAAGCCA GGCTAGCTACAACGA AGTGTGCG | 8093 |
| 5113 | ACACUGUG G CUUGGUAU | 3297 | ATACCAAG GGCTAGCTACAACGA CACAGTGT | 8094 |
| 5108 | GUGGCUUG G UAUGCUAC | 3298 | GTAGCATA GGCTAGCTACAACGA CAAGCCAC | 8095 |
| 5106 | GGCUUGGU A UGCUACCA | 3299 | TGGTAGCA GGCTAGCTACAACGA ACCAAGCC | 8096 |
| 5104 | CUUGGUAU G CUACCAGG | 3300 | CCTGGTAG GGCTAGCTACAACGA ATACCAAG | 8097 |
| 5101 | GGUAUGCU A CCAGGUAG | 3301 | CTACCTGG GGCTAGCTACAACGA AGCATACC | 8098 |
| 5096 | GCUACCAG G UAGGGGAG | 3302 | CTCCCCTA GGCTAGCTACAACGA CTGGTAGC | 8099 |
| 5087 | UAGGGGAG G UUUUCUCC | 3303 | GGAGAAAA GGCTAGCTACAACGA CTCCCCTA | 8100 |
| 5077 | UUUCUCCU G CCUGCUUG | 3304 | CAAGCAGG GGCTAGCTACAACGA AGGAGAAA | 8101 |
| 5073 | UCCUGCCU G CUUGGUCU | 3305 | AGACCAAG GGCTAGCTACAACGA AGGCAGGA | 8102 |
| 5068 | CCUGCUUG G UCUGGGAC | 3306 | GTCCCAGA GGCTAGCTACAACGA CAAGCAGG | 8103 |
| 5061 | GGUCUGGG A CAAGAAGU | 3307 | ACTTCTTG GGCTAGCTACAACGA CCCAGACC | 8104 |
| 5054 | GACAAGAA G UGGGCAUC | 3308 | GATGCCCA GGCTAGCTACAACGA TTCTTGTC | 8105 |
| 5050 | AGAAGUGG G CAUCUAUG | 3309 | CATAGATG GGCTAGCTACAACGA CCACTTCT | 8106 |
| 5048 | AAGUGGGC A UCUAUGUG | 3310 | CACATAGA GGCTAGCTACAACGA GCCCACTT | 8107 |
| 5044 | GGGCAUCU A UGUGGGUG | 3311 | CACCCACA GGCTAGCTACAACGA AGATGCCC | 8108 |
| 5042 | GCAUCUAU G UGGGUGAG | 3312 | CTCACCCA GGCTAGCTACAACGA ATAGATGC | 8109 |
| 5038 | CUAUGUGG G UGAGGCCU | 3313 | AGGCCTCA GGCTAGCTACAACGA CCACATAG | 8110 |
| 5033 | UGGGUGAG G CCUGUGAA | 3314 | TTCACAGG GGCTAGCTACAACGA CTCACCCA | 8111 |
| 5029 | UGAGGCCU G UGAAGACA | 3315 | TGTCTTCA GGCTAGCTACAACGA AGGCCTCA | 8112 |
| 5023 | CUGUGAAG A CACCCUCC | 3316 | GGAGGGTG GGCTAGCTACAACGA CTTCACAG | 8113 |
| 5021 | GUGAAGAC A CCCUCCCA | 3317 | TGGGAGGG GGCTAGCTACAACGA GTCTTCAC | 8114 |
| 5010 | CUCCCAGA A CUCCAGAU | 3318 | ATCTGGAG GGCTAGCTACAACGA TCTGGGAG | 8115 |
| 5003 | AACUCCAG A UGGUCCUG | 3319 | CAGGACCA GGCTAGCTACAACGA CTGGAGTT | 8116 |
| 5000 | UCCAGAUG G UCCUGGCA | 3320 | TGCCAGGA GGCTAGCTACAACGA CATCTGGA | 8117 |
| 4994 | UGGUCCUG G CAGAAGGG | 3321 | CCCTTCTG GGCTAGCTACAACGA CAGGACCA | 8118 |
| 4986 | GCAGAAGG G CAACCCUG | 3322 | CAGGGTTG GGCTAGCTACAACGA CCTTCTGC | 8119 |
| 4983 | GAAGGGCA A CCCUGGUG | 3323 | CACCAGGG GGCTAGCTACAACGA TGCCCTTC | 8120 |
| 4977 | CAACCCUG G UGUAUUUA | 3324 | TAAATACA GGCTAGCTACAACGA CAGGGTTG | 8121 |
| 4975 | ACCCUGGU G UAUUUAGG | 3325 | CCTAAATA GGCTAGCTACAACGA ACCAGGGT | 8122 |
| 4973 | CCUGGUGU A UUUAGGUA | 3326 | TACCTAAA GGCTAGCTACAACGA ACACCAGG | 8123 |
| 4967 | GUAUUUAG G UAAGCCCG | 3327 | CGGGCTTA GGCTAGCTACAACGA CTAAATAC | 8124 |
| 4963 | UUAGGUAA G CCCGCAAC | 3328 | GTTGCGGG GGCTAGCTACAACGA TTACCTAA | 8125 |
| 4959 | GUAAGCCC G CAACCUAA | 3329 | TTAGGTTG GGCTAGCTACAACGA GGGCTTAC | 8126 |
| 4956 | AGCCCGCA A CCUAACGG | 3330 | CCGTTAGG GGCTAGCTACAACGA TGCGGGCT | 8127 |
| 4951 | GCAACCUA A CGGAGGUC | 3331 | GACCTCCG GGCTAGCTACAACGA TAGGTTGC | 8128 |
| 4945 | UAACGGAG G UCUCGGCG | 3332 | CGCCGAGA GGCTAGCTACAACGA CTCCGTTA | 8129 |
| 4939 | AGGUCUCG G CGGGCGUG | 3333 | CACGCCCG GGCTAGCTACAACGA CGAGACCT | 8130 |
| 4935 | UAACGGAG G UCUCGGCG | 3334 | AGCTCACG GGCTAGCTACAACGA CCGCCGAG | 8131 |
| 4933 | CGGCGGGC G UGAGCUCG | 3335 | CGAGCTCA GGCTAGCTACAACGA GCCCGCCG | 8132 |
| 4929 | GGGCGUGA G CUCGUACC | 3336 | GGTACGAG GGCTAGCTACAACGA TCACGCCC | 8133 |
| 4925 | GUGAGCUC G UACCAAGC | 3337 | GCTTGGTA GGCTAGCTACAACGA GAGCTCAC | 8134 |
| 4923 | GAGCUCGU A CCAAGCAC | 3338 | GTGCTTGG GGCTAGCTACAACGA ACGAGCTC | 8135 |
| 4918 | CGUACCAA G CACAUCCC | 3339 | GGGATGTG GGCTAGCTACAACGA TTGGTACG | 8136 |
| 4916 | UACCAAGC A CAUCCCGC | 3340 | GCGGGATG GGCTAGCTACAACGA GCTTGGTA | 8137 |
| 4914 | CCAAGCAC A UCCCGCGU | 3341 | ACGCGGGA GGCTAGCTACAACGA GTGCTTGG | 8138 |
| 4909 | CACAUCCC G CGUCAUAG | 3342 | CTATGACG GGCTAGCTACAACGA GGGATGTG | 8139 |
| 4907 | CAUCCCGC G UCAUAGCA | 3343 | TGCTATGA GGCTAGCTACAACGA GCGGGATG | 8140 |
| 4904 | CCCGCGUC A UAGCACUC | 3344 | GAGTGCTA GGCTAGCTACAACGA GACGCGGG | 8141 |
| 4901 | GCGUCAUA G CACUCACA | 3345 | TGTGAGTG GGCTAGCTACAACGA TATGACGC | 8142 |
| 4899 | GUCAUAGC A CUCACACA | 3346 | TGTGTGAG GGCTAGCTACAACGA GCTATGAC | 8143 |
| 4895 | UAGCACUC A CACAGGAC | 3347 | GTCCTGTG GGCTAGCTACAACGA GAGTGCTA | 8144 |
| 4893 | GCACUCAC A CAGGACCG | 3348 | CGGTCCTG GGCTAGCTACAACGA GTGAGTGC | 8145 |
| 4888 | CACACAGG A CCGAGGAG | 3349 | CTCCTCGG GGCTAGCTACAACGA CCTGTGTG | 8146 |
| 4880 | ACCGAGGA G UCGAACAU | 3350 | ATGTTCGA GGCTAGCTACAACGA TCCTCGGT | 8147 |
| 4875 | GGAGUCGA A CAUGCCCG | 3351 | CGGGCATG GGCTAGCTACAACGA TCGACTCC | 8148 |
| 4873 | AGUCGAAC A UGCCCGAA | 3352 | TTCGGGCA GGCTAGCTACAACGA GTTCGACT | 8149 |
| 4871 | UCGAACAU G CCCGAAGG | 3353 | CCTTCGGG GGCTAGCTACAACGA ATGTTCGA | 8150 |
| 4863 | GCCCGAAG G CCGCUCUC | 3354 | GAGAGCGG GGCTAGCTACAACGA CTTCGGGC | 8151 |
| 4860 | CGAAGGCC G CUCUCCUG | 3355 | CAGGAGAG GGCTAGCTACAACGA GGCCTTCG | 8152 |
| 4849 | CUCCUGGA G UCACAAAC | 3356 | GTTTGTGA GGCTAGCTACAACGA TCCAGGAG | 8153 |
| 4846 | CUGGAGUC A CAAACCUG | 3357 | CAGGTTTG GGCTAGCTACAACGA GACTCCAG | 8154 |
| 4842 | AGUCACAA A CCUGUAUA | 3358 | TATACAGG GGCTAGCTACAACGA TTGTGACT | 8155 |
| 4838 | ACAAACCU G UAUAUGCC | 3359 | GGCATATA GGCTAGCTACAACGA AGGTTTGT | 8156 |
| 4836 | AAACCUGU A UAUGCCUC | 3360 | GAGGCATA GGCTAGCTACAACGA ACAGGTTT | 8157 |
| 4834 | ACCUGUAU A UGCCUCUC | 3361 | GAGAGGCA GGCTAGCTACAACGA ATACAGGT | 8158 |
| 4832 | CUGUAUAU G CCUCUCCU | 3362 | AGGAGAGG GGCTAGCTACAACGA ATATACAG | 8159 |
| 4823 | CCUCUCCU G CCCCUACC | 3363 | GGTAGGGG GGCTAGCTACAACGA AGGAGAGG | 8160 |
| 4817 | CUGCCCCU A CCGGUCCU | 3364 | AGGACCGG GGCTAGCTACAACGA AGGGGCAG | 8161 |
| 4813 | CCCUACCG G UCCUACCU | 3365 | AGGTAGGA GGCTAGCTACAACGA CGGTAGGG | 8162 |
| 4808 | CCGGUCCU A CCUCGCCU | 3366 | AGGCGAGG GGCTAGCTACAACGA AGGACCGG | 8163 |
| 4803 | CCUACCUC G CCUCUGCG | 3367 | CGCAGAGG GGCTAGCTACAACGA GAGGTAGG | 8164 |
| 4797 | UCGCCUCU G CGAGCGGG | 3368 | CCCGCTCG GGCTAGCTACAACGA AGAGGCGA | 8165 |
| 4793 | CUCUGCGA G CGGGACAC | 3369 | GTGTCCCG GGCTAGCTACAACGA TCGCAGAG | 8166 |
| 4788 | CGAGCGGG A CACUGCGU | 3370 | ACGCAGTG GGCTAGCTACAACGA CCCGCTCG | 8167 |
| 4786 | AGCGGGAC A CUGCGUCU | 3371 | AGACGCAG GGCTAGCTACAACGA GTCCCGCT | 8168 |
| 4783 | GGGACACU G CGUCUUGG | 3372 | CCAAGACG GGCTAGCTACAACGA AGTGTCCC | 8169 |
| 4781 | GACACUGC G UCUUGGGG | 3373 | CCCCAAGA GGCTAGCTACAACGA GCAGTGTC | 8170 |
| 4773 | GUCUUGGG G CACGGUCG | 3374 | CGACCGTG GGCTAGCTACAACGA CCCAAGAC | 8171 |
| 4771 | CUUGGGGC A CGGUCGUC | 3375 | GACGACCG GGCTAGCTACAACGA GCCCCAAG | 8172 |
| 4768 | GGGGCACG G UCGUCGUC | 3376 | GACGACGA GGCTAGCTACAACGA CGTGCCCC | 8173 |
| 4765 | GCACGGUC G UCGUCUCA | 3377 | TGAGACGA GGCTAGCTACAACGA GACCGTGC | 8174 |
| 4762 | CGGUCGUC G UCUCAAUG | 3378 | CATTGAGA GGCTAGCTACAACGA GACGACCG | 8175 |
| 4756 | UCGUCUCA A UGGUGAAG | 3379 | CTTCACCA GGCTAGCTACAACGA TGAGACGA | 8176 |
| 4753 | UCUCAAUG G UGAAGGUA | 3380 | TACCTTCA GGCTAGCTACAACGA CATTGAGA | 8177 |
| 4747 | UGGUGAAG G UAGGGUCC | 3381 | GGACCCTA GGCTAGCTACAACGA CTTCACCA | 8178 |
| 4742 | AAGGUAGG G UCCAAGCU | 3382 | AGCTTGGA GGCTAGCTACAACGA CCTACCTT | 8179 |
| 4736 | GGGUCCAA G CUGAAGUC | 3383 | GACTTCAG GGCTAGCTACAACGA TTGGACCC | 8180 |
| 4730 | AAGCUGAA G UCGACUGU | 3384 | ACAGTCGA GGCTAGCTACAACGA TTCAGCTT | 8181 |
| 4726 | UGAAGUCG A CUGUUUGG | 3385 | CCAAACAG GGCTAGCTACAACGA CGACTTCA | 8182 |
| 4723 | AGUCGACU G UUUGGGUG | 3386 | CACCCAAA GGCTAGCTACAACGA AGTCGACT | 8183 |
| 4717 | CUGUUUGG G UGACACAU | 3387 | ATGTGTCA GGCTAGCTACAACGA CCAAACAG | 8184 |
| 4714 | UUUGGGUG A CACAUGUA | 3388 | TACATGTG GGCTAGCTACAACGA CACCCAAA | 8185 |
| 4712 | UGGGUGAC A CAUGUAUU | 3389 | AATACATG GGCTAGCTACAACGA GTCACCCA | 8186 |
| 4710 | GGUGACAC A UGUAUUAC | 3390 | GTAATACA GGCTAGCTACAACGA GTGTCACC | 8187 |
| 4708 | UGACACAU G UAUUACAG | 3391 | CTGTAATA GGCTAGCTACAACGA ATGTGTCA | 8188 |
| 4706 | ACACAUGU A UUACAGUC | 3392 | GACTGTAA GGCTAGCTACAACGA ACATGTGT | 8189 |
| 4703 | CAUGUAUU A CAGUCGAU | 3393 | ATCGACTG GGCTAGCTACAACGA AATACATG | 8190 |
| 4700 | GUAUUACA G UCGAUCAC | 3394 | GTGATCGA GGCTAGCTACAACGA TGTAATAC | 8191 |
| 4696 | UACAGUCG A UCACCGAG | 3395 | CTCGGTGA GGCTAGCTACAACGA CGACTGTA | 8192 |
| 4693 | AGUCGAUC A CCGAGUCA | 3396 | TGACTCGG GGCTAGCTACAACGA GATCGACT | 8193 |
| 4688 | AUCACCGA G UCAAAAUC | 3397 | GATTTTGA GGCTAGCTACAACGA TCGGTGAT | 8194 |
| 4682 | GAGUCAAA A UCGCCGGU | 3398 | ACCGGCGA GGCTAGCTACAACGA TTTGACTC | 8195 |
| 4679 | UCAAAAUC G CCGGUAUA | 3399 | TATACCGG GGCTAGCTACAACGA GATTTTGA | 8196 |
| 4675 | AAUCGCCG G UAUAGCCC | 3400 | GGGCTATA GGCTAGCTACAACGA CGGCGATT | 8197 |
| 4673 | UCGCCGGU A UAGCCCGU | 3401 | ACGGGCTA GGCTAGCTACAACGA ACCGGCGA | 8198 |
| 4670 | CCGGUAUA G CCCGUCAU | 3402 | ATGACGGG GGCTAGCTACAACGA TATACCGG | 8199 |
| 4666 | UAUAGCCC G UCAUUAGA | 3403 | TCTAATGA GGCTAGCTACAACGA GGGCTATA | 8200 |
| 4663 | AGCCCGUC A UUAGAGCG | 3404 | CGCTCTAA GGCTAGCTACAACGA GACGGGCT | 8201 |
| 4657 | UCAUUAGA G CGUCUGUU | 3405 | AACAGACG GGCTAGCTACAACGA TCTAATGA | 8202 |
| 4655 | AUUAGAGC G UCUGUUGC | 3406 | GCAACAGA GGCTAGCTACAACGA GCTCTAAT | 8203 |
| 4651 | GAGCGUCU G UUGCCACG | 3407 | CGTGGCAA GGCTAGCTACAACGA AGACGCTC | 8204 |
| 4648 | CGUCUGUU G CCACGACA | 3408 | TGTCGTGG GGCTAGCTACAACGA AACAGACG | 8205 |
| 4645 | CUGUUGCC A CGACAACG | 3409 | CGTTGTCG GGCTAGCTACAACGA GGCAACAG | 8206 |
| 4642 | UUGCCACG A CAACGACG | 3410 | CGTCGTTG GGCTAGCTACAACGA CGTGGCAA | 8207 |
| 4639 | CCACGACA A CGACGUCC | 3411 | GGACGTCG GGCTAGCTACAACGA TGTCGTGG | 8208 |
| 4636 | CGACAACG A CGUCCCCG | 3412 | CGGGGACG GGCTAGCTACAACGA CGTTGTCG | 8209 |
| 4634 | ACAACGAC G UCCCCGCU | 3413 | AGCGGGGA GGCTAGCTACAACGA GTCGTTGT | 8210 |
| 4628 | ACGUCCCC G CUGGCCGG | 3414 | CCGGCCAG GGCTAGCTACAACGA GGGGACGT | 8211 |
| 4624 | CCCCGCUG G CCGGUAUG | 3415 | CATACCGG GGCTAGCTACAACGA CAGCGGGG | 8212 |
| 4620 | GCUGGCCG G UAUGACGG | 3416 | CCGTCATA GGCTAGCTACAACGA CGGCCAGC | 8213 |
| 4618 | UGGCCGGU A UGACGGAC | 3417 | GTCCGTCA GGCTAGCTACAACGA ACCGGCCA | 8214 |
| 4615 | CCGGUAUG A CGGACACG | 3418 | CGTGTCCG GGCTAGCTACAACGA CATACCGG | 8215 |
| 4611 | UAUGACGG A CACGUCGA | 3419 | TCGACGTG GGCTAGCTACAACGA CCGTCATA | 8216 |
| 4609 | UGACGGAC A CGUCGAGA | 3420 | TCTCGACG GGCTAGCTACAACGA GTCCGTCA | 8217 |
| 4607 | ACGGACAC G UCGAGACC | 3421 | GGTCTCGA GGCTAGCTACAACGA GTGTCCGT | 8218 |
| 4601 | ACGUCGAG A CCCCGGUA | 3422 | TACCGGGG GGCTAGCTACAACGA CTCGACGT | 8219 |
| 4595 | AGACCCCG G UAAUACGC | 3423 | GCGTATTA GGCTAGCTACAACGA CGGGGTCT | 8220 |
| 4592 | CCCCGGUA A UACGCUAC | 3424 | GTAGCGTA GGCTAGCTACAACGA TACCGGGG | 8221 |
| 4590 | CCGGUAAU A CGCUACAG | 3425 | CTGTAGCG GGCTAGCTACAACGA ATTACCGG | 8222 |
| 4588 | GGUAAUAC G CUACAGCG | 3426 | CGCTGTAG GGCTAGCTACAACGA GTATTACC | 8223 |
| 4585 | AAUACGCU A CAGCGUUA | 3427 | TAACGCTG GGCTAGCTACAACGA AGCGTATT | 8224 |
| 4582 | ACGCUACA G CGUUAAGU | 3428 | ACTTAACG GGCTAGCTACAACGA TGTAGCGT | 8225 |
| 4580 | GCUACAGC G UUAAGUCC | 3429 | GGACTTAA GGCTAGCTACAACGA GCTGTAGC | 8226 |
| 4575 | AGCGUUAA G UCCGAGGC | 3430 | GCCTCGGA GGCTAGCTACAACGA TTAACGCT | 8227 |
| 4568 | AGUCCGAG G CCCGACAG | 3431 | CTGTCGGG GGCTAGCTACAACGA CTCGGACT | 8228 |
| 4563 | GAGGCCCG A CAGCUUUG | 3432 | CAAAGCTG GGCTAGCTACAACGA CGGGCCTC | 8229 |
| 4560 | GCCCGACA G CUUUGCAG | 3433 | CTGCAAAG GGCTAGCTACAACGA TGTCGGGC | 8230 |
| 4555 | ACAGCUUU G CAGCGAGC | 3434 | GCTCGCTG GGCTAGCTACAACGA AAAGCTGT | 8231 |
| 4552 | GCUUUGCA G CGAGCUCG | 3435 | CGAGCTCG GGCTAGCTACAACGA TGCAAAGC | 8232 |
| 4548 | UGCAGCGA G CUCGUCAC | 3436 | GTGACGAG GGCTAGCTACAACGA TCGCTGCA | 8233 |
| 4544 | GCGAGCUC G UCACAUUU | 3437 | AAATGTGA GGCTAGCTACAACGA GAGCTCGC | 8234 |
| 4541 | AGCUCGUC A CAUUUCUU | 3438 | AAGAAATG GGCTAGCTACAACGA GACGAGCT | 8235 |
| 4539 | CUCGUCAC A UUUCUUCU | 3439 | AGAAGAAA GGCTAGCTACAACGA GTGACGAG | 8236 |
| 4526 | UUCUUGGA A UGGCAGAA | 3440 | TTCTGCCA GGCTAGCTACAACGA TCCAAGAA | 8237 |
| 4523 | UUGGAAUG G CAGAAGAU | 3441 | ATCTTCTG GGCTAGCTACAACGA CATTCCAA | 8238 |
| 4516 | GGCAGAAG A UGAGAUGC | 3442 | GCATCTCA GGCTAGCTACAACGA CTTCTGCC | 8239 |
| 4511 | AAGAUGAG A UGCCUCCC | 3443 | GGGAGGCA GGCTAGCTACAACGA CTCATCTT | 8240 |
| 4509 | GAUGAGAU G CCUCCCCC | 3444 | GGGGGAGG GGCTAGCTACAACGA ATCTCATC | 8241 |
| 4495 | CCCCUUUG A UGGUCUCG | 3445 | CGAGACCA GGCTAGCTACAACGA CAAAGGGG | 8242 |
| 4492 | CUUUGAUG G UCUCGAUG | 3446 | CATCGAGA GGCTAGCTACAACGA CATCAAAG | 8243 |
| 4486 | UGGUCUCG A UGGGGAUG | 3447 | CATCCCCA GGCTAGCTACAACGA CGAGACCA | 8244 |
| 4480 | CGAUGGGG A UGGCUUUG | 3448 | CAAAGCCA GGCTAGCTACAACGA CCCCATCG | 8245 |
| 4477 | UGGGGAUG G CUUUGCCA | 3449 | TGGCAAAG GGCTAGCTACAACGA CATCCCCA | 8246 |
| 4472 | AUGGCUUU G CCAUAGAA | 3450 | TTCTATGG GGCTAGCTACAACGA AAAGCCAT | 8247 |
| 4469 | GCUUUGCC A UAGAAGGG | 3451 | CCCTTCTA GGCTAGCTACAACGA GGCAAAGC | 8248 |
| 4459 | AGAAGGGG A UCUCUCCG | 3452 | CGGAGAGA GGCTAGCTACAACGA CCCCTTCT | 8249 |
| 4450 | UCUCUCCG G UGUUGGAC | 3453 | GTCCAACA GGCTAGCTACAACGA CGGAGAGA | 8250 |
| 4448 | UCUCCGGU G UUGGACAA | 3454 | TTGTCCAA GGCTAGCTACAACGA ACCGGAGA | 8251 |
| 4443 | GGUGUUGG A CAAGGCUA | 3455 | TAGCCTTG GGCTAGCTACAACGA CCAACACC | 8252 |
| 4438 | UGGACAAG G CUAUCUCC | 3456 | GGAGATAG GGCTAGCTACAACGA CTTGTCCA | 8253 |
| 4435 | ACAAGGCU A UCUCCUCG | 3457 | CGAGGAGA GGCTAGCTACAACGA AGCCTTGT | 8254 |
| 4426 | UCUCCUCG A UGUUGGGA | 3458 | TCCCAACA GGCTAGCTACAACGA CGAGGAGA | 8255 |
| 4424 | UCCUCGAU G UUGGGAUG | 3459 | CATCCCAA GGCTAGCTACAACGA ATCGAGGA | 8256 |
| 4418 | AUGUUGGG A UGUGGCAC | 3460 | GTGCCACA GGCTAGCTACAACGA CCCAACAT | 8257 |
| 4416 | GUUGGGAU G UGGCACGG | 3461 | CCGTGCCA GGCTAGCTACAACGA ATCCCAAC | 8258 |
| 4413 | GGGAUGUG G CACGGUGA | 3462 | TCACCGTG GGCTAGCTACAACGA CACATCCC | 8259 |
| 4411 | GAUGUGGC A CGGUGACC | 3463 | GGTCACCG GGCTAGCTACAACGA GCCACATC | 8260 |
| 4408 | GUGGCACG G UGACCGAU | 3464 | ATCGGTCA GGCTAGCTACAACGA CGTGCCAC | 8261 |
| 4405 | GCACGGUG A CCGAUCCC | 3465 | GGGATCGG GGCTAGCTACAACGA CACCGTGC | 8262 |
| 4401 | GGUGACCG A UCCCGGAG | 3466 | CTCCGGGA GGCTAGCTACAACGA CGGTCACC | 8263 |
| 4392 | UCCCGGAG G CGUAGCGG | 3467 | CCGCTACG GGCTAGCTACAACGA CTCCGGGA | 8264 |
| 4390 | CCGGAGGC G UAGCGGUG | 3468 | CACCGCTA GGCTAGCTACAACGA GCCTCCGG | 8265 |
| 4387 | GAGGCGUA G CGGUGGCG | 3469 | CGCCACCG GGCTAGCTACAACGA TACGCCTC | 8266 |
| 4384 | GCGUAGCG G UGGCGAGC | 3470 | GCTCGCCA GGCTAGCTACAACGA CGCTACGC | 8267 |
| 4381 | UAGCGGUG G CGAGCACG | 3471 | CGTGCTCG GGCTAGCTACAACGA CACCGCTA | 8268 |
| 4377 | GGUGGCGA G CACGACGA | 3472 | TCGTCGTG GGCTAGCTACAACGA TCGCCACC | 8269 |
| 4375 | UGGCGAGC A CGACGAGC | 3473 | GCTCGTCG GGCTAGCTACAACGA GCTCGCCA | 8270 |
| 4372 | CGAGCACG A CGAGCCGC | 3474 | GCGGCTCG GGCTAGCTACAACGA CGTGCTCG | 8271 |
| 4368 | CACGACGA G CCGCGCUC | 3475 | GAGCGCGG GGCTAGCTACAACGA TCGTCGTG | 8272 |
| 4365 | GACGAGCC G CGCUCCAG | 3476 | CTGGAGCG GGCTAGCTACAACGA GGCTCGTC | 8273 |
| 4363 | CGAGCCGC G CUCCAGCC | 3477 | GGCTGGAG GGCTAGCTACAACGA GCGGCTCG | 8274 |
| 4357 | GCGCUCCA G CCGUCUCC | 3478 | GGAGACGG GGCTAGCTACAACGA TGGAGCGC | 8275 |
| 4354 | CUCCAGCC G UCUCCGCU | 3479 | AGCGGAGA GGCTAGCTACAACGA GGCTGGAG | 8276 |
| 4348 | CCGUCUCC G CUUGGUCC | 3480 | GGACCAAG GGCTAGCTACAACGA GGAGACGG | 8277 |
| 4343 | UCCGCUUG G UCCAGGAC | 3481 | GTCCTGGA GGCTAGCTACAACGA CAAGCGGA | 8278 |
| 4336 | GGUCCAGG A CUGUGCCG | 3482 | CGGCACAG GGCTAGCTACAACGA CCTGGACC | 8279 |
| 4333 | CCAGGACU G UGCCGAUG | 3483 | CATCGGCA GGCTAGCTACAACGA AGTCCTGG | 8280 |
| 4331 | AGGACUGU G CCGAUGCC | 3484 | GGCATCGG GGCTAGCTACAACGA ACAGTCCT | 8281 |
| 4327 | CUGUGCCG A UGCCCAAA | 3485 | TTTGGGCA GGCTAGCTACAACGA CGGCACAG | 8282 |
| 4325 | GUGCCGAU G CCCAAAAU | 3486 | ATTTTGGG GGCTAGCTACAACGA ATCGGCAC | 8283 |
| 4318 | UGCCCAAA A UGGAAGUC | 3487 | GACTTCCA GGCTAGCTACAACGA TTTGGGCA | 8284 |
| 4312 | AAAUGGAA G UCGAGUCA | 3488 | TGACTCGA GGCTAGCTACAACGA TTCCATTT | 8285 |
| 4307 | GAAGUCGA G UCAAUUGA | 3489 | TCAATTGA GGCTAGCTACAACGA TCGACTTC | 8286 |
| 4303 | UCGAGUCA A UUGAGUGG | 3490 | CCACTCAA GGCTAGCTACAACGA TGACTCGA | 8287 |
| 4298 | UCAAUUGA G UGGCACUC | 3491 | GAGTGCCA GGCTAGCTACAACGA TCAATTGA | 8288 |
| 4295 | AUUGAGUG G CACUCAUC | 3492 | GATGAGTG GGCTAGCTACAACGA CACTCAAT | 8289 |
| 4293 | UGAGUGGC A CUCAUCAC | 3493 | GTGATGAG GGCTAGCTACAACGA GCCACTCA | 8290 |
| 4289 | UGGCACUC A UCACACAU | 3494 | ATGTGTGA GGCTAGCTACAACGA GAGTGCCA | 8291 |
| 4286 | CACUCAUC A CACAUUAU | 3495 | ATAATGTG GGCTAGCTACAACGA GATGAGTG | 8292 |
| 4284 | CUCAUCAC A CAUUAUGA | 3496 | TCATAATG GGCTAGCTACAACGA GTGATGAG | 8293 |
| 4282 | CAUCACAC A UUAUGAUG | 3497 | CATCATAA GGCTAGCTACAACGA GTGTGATG | 8294 |
| 4279 | CACACAUU A UGAUGUCA | 3498 | TGACATCA GGCTAGCTACAACGA AATGTGTG | 8295 |
| 4276 | ACAUUAUG A UGUCAUAG | 3499 | CTATGACA GGCTAGCTACAACGA CATAATGT | 8296 |
| 4274 | AUUAUGAU G UCAUAGGC | 3500 | GCCTATGA GGCTAGCTACAACGA ATCATAAT | 8297 |
| 4271 | AUGAUGUC A UAGGCGCC | 3501 | GGCGCCTA GGCTAGCTACAACGA GACATCAT | 8298 |
| 4267 | UGUCAUAG G CGCCCCCA | 3502 | TGGGGGCG GGCTAGCTACAACGA CTATGACA | 8299 |
| 4265 | UCAUAGGC G CCCCCAGA | 3503 | TCTGGGGG GGCTAGCTACAACGA GCCTATGA | 8300 |
| 4256 | CCCCCAGA G CAACCACC | 3504 | GGTGGTTG GGCTAGCTACAACGA TCTGGGGG | 8301 |
| 4253 | CCAGAGCA A CCACCGUC | 3505 | GACGGTGG GGCTAGCTACAACGA TGCTCTGG | 8302 |
| 4250 | GAGCAACC A CCGUCGGC | 3506 | GCCGACGG GGCTAGCTACAACGA GGTTGCTC | 8303 |
| 4247 | CAACCACC G UCGGCAAG | 3507 | CTTGCCGA GGCTAGCTACAACGA GGTGGTTG | 8304 |
| 4243 | CACCGUCG G CAAGGAAC | 3508 | GTTCCTTG GGCTAGCTACAACGA CGACGGTG | 8305 |
| 4236 | GGCAAGGA A CUUGCCAU | 3509 | ATGGCAAG GGCTAGCTACAACGA TCCTTGCC | 8306 |
| 4232 | AGGAACUU G CCAUAGGU | 3510 | ACCTATGG GGCTAGCTACAACGA AAGTTCCT | 8307 |
| 4229 | AACUUGCC A UAGGUGGA | 3511 | TCCACCTA GGCTAGCTACAACGA GGCAAGTT | 8308 |
| 4225 | UGCCAUAG G UGGAGUAC | 3512 | GTACTCCA GGCTAGCTACAACGA CTATGGCA | 8309 |
| 4220 | UAGGUGGA G UACGUGAU | 3513 | ATCACGTA GGCTAGCTACAACGA TCCACCTA | 8310 |
| 4218 | GGUGGAGU A CGUGAUGG | 3514 | CCATCACG GGCTAGCTACAACGA ACTCCACC | 8311 |
| 4216 | UGGAGUAC G UGAUGGGG | 3515 | CCCCATCA GGCTAGCTACAACGA GTACTCCA | 8312 |
| 4213 | AGUACGUG A UGGGGGCG | 3516 | CGCCCCCA GGCTAGCTACAACGA CACGTACT | 8313 |
| 4207 | UGAUGGGG G CGCCCGUG | 3517 | CACGGGCG GGCTAGCTACAACGA CCCCATCA | 8314 |
| 4205 | AUGGGGGC G CCCGUGGU | 3518 | ACCACGGG GGCTAGCTACAACGA GCCCCCAT | 8315 |
| 4201 | GGGCGCCC G UGGUGAUG | 3519 | CATCACCA GGCTAGCTACAACGA GGGCGCCC | 8316 |
| 4198 | CGCCCGUG G UGAUGGUC | 3520 | GACCATCA GGCTAGCTACAACGA CACGGGCG | 8317 |
| 4195 | CCGUGGUG A UGGUCCUU | 3521 | AAGGACCA GGCTAGCTACAACGA CACCACGG | 8318 |
| 4192 | UGGUGAUG G UCCUUACC | 3522 | GGTAAGGA GGCTAGCTACAACGA CATCACCA | 8319 |
| 4186 | UGGUCCUU A CCCCAGUU | 3523 | AACTGGGG GGCTAGCTACAACGA AAGGACCA | 8320 |
| 4180 | UUACCCCA G UUCUGAUG | 3524 | CATCAGAA GGCTAGCTACAACGA TGGGGTAA | 8321 |
| 4174 | CAGUUCUG A UGUUAGGA | 3525 | TCCTAACA GGCTAGCTACAACGA CAGAACTG | 8322 |
| 4172 | GUUCUGAU G UUAGGAUC | 3526 | GATCCTAA GGCTAGCTACAACGA ATCAGAAC | 8323 |
| 4166 | AUGUUAGG A UCGACACC | 3527 | GGTGTCGA GGCTAGCTACAACGA CCTAACAT | 8324 |
| 4162 | UAGGAUCG A CACCGUGU | 3528 | ACACGGTG GGCTAGCTACAACGA CGATCCTA | 8325 |
| 4160 | GGAUCGAC A CCGUGUGC | 3529 | GCACACGG GGCTAGCTACAACGA TGCGATCC | 8326 |
| 4157 | UCGACACC G UGUGCCUU | 3530 | AAGGCACA GGCTAGCTACAACGA GGTGTCGA | 8327 |
| 4155 | GACACCGU G UGCCUUAG | 3531 | CTAAGGCA GGCTAGCTACAACGA ACGGTGTC | 8328 |
| 4253 | CACCGUGU G CCUUAGAC | 3532 | GTCTAAGG GGCTAGCTACAACGA ACACGGTG | 8329 |
| 4246 | UGCCUUAG A CAUAUACG | 3533 | CGTATATG GGCTAGCTACAACGA CTAAGGCA | 8330 |
| 4144 | CCUUAGAC A UAUACGCC | 3534 | GGCGTATA GGCTAGCTACAACGA GTCTAAGG | 8331 |
| 4142 | UUAGACAU A UACGCCCC | 3535 | GGGGCGTA GGCTAGCTACAACGA ATGTCTAA | 8332 |
| 4140 | AGACAUAU A CGCCCCAA | 3536 | TTGGGGCG GGCTAGCTACAACGA ATATGTCT | 8333 |
| 4138 | ACAUAUAC G CCCCAAAC | 3537 | GTTTGGGG GGCTAGCTACAACGA GTATATGT | 8334 |
| 4131 | CGCCCCAA A CCCUAAGG | 3538 | CCTTAGGG GGCTAGCTACAACGA TTGGGGCG | 8335 |
| 4123 | ACCCUAAG G UGGCGGUA | 3539 | TACCGCCA GGCTAGCTACAACGA CTTAGGGT | 8336 |
| 4120 | CUAAGGUG G CGGUAACG | 3540 | CGTTACCG GGCTAGCTACAACGA CACCTTAG | 8337 |
| 4117 | AGGUGGCG G UAACGGAC | 3541 | GTCCGTTA GGCTAGCTACAACGA CGCCACCT | 8338 |
| 4114 | UGGCGGUA A CGGACGGA | 3542 | TCCGTCCG GGCTAGCTACAACGA TACCGCCA | 8339 |
| 4110 | GGUAACGG A CGGAUUUA | 3543 | TAAATCCG GGCTAGCTACAACGA CCGTTACC | 8340 |
| 4106 | ACGGACGG A UUUAGGAC | 3544 | GTCCTAAA GGCTAGCTACAACGA CCGTCCGT | 8341 |
| 4099 | GAUUUAGG A CGAGCACU | 3545 | AGTGCTCG GGCTAGCTACAACGA CCTAAATC | 8342 |
| 4095 | UAGGACGA G CACUUUGU | 3546 | ACAAAGTG GGCTAGCTACAACGA TCGTCCTA | 8343 |
| 4093 | GGACGAGC A CUUUGUAC | 3547 | GTACAAAG GGCTAGCTACAACGA GCTCGTCC | 8344 |
| 4088 | AGCACUUU G UACCCUUG | 3548 | CAAGGGTA GGCTAGCTACAACGA AAAGTGCT | 8345 |
| 4086 | CACUUUGU A CCCUUGGG | 3549 | CCCAAGGG GGCTAGCTACAACGA ACAAAGTG | 8346 |
| 4078 | ACCCUUGG G CUGCAUAU | 3550 | ATATGCAG GGCTAGCTACAACGA CCAAGGGT | 8347 |
| 4075 | CUUGGGCU G CAUAUGCA | 3551 | TGCATATG GGCTAGCTACAACGA AGCCCAAG | 8348 |
| 4073 | UGGGCUGC A UAUGCAGC | 3552 | GCTGCATA GGCTAGCTACAACGA GCAGCCCA | 8349 |
| 4071 | GGCUGCAU A UGCAGCCG | 3553 | CGGCTGCA GGCTAGCTACAACGA ATGCAGCC | 8350 |
| 4069 | CUGCAUAU G CAGCCGGU | 3554 | ACCGGCTG GGCTAGCTACAACGA ATATGCAG | 8351 |
| 4066 | CAUAUGCA G CCGGUACC | 3555 | GGTACCGG GGCTAGCTACAACGA TGCATATG | 8352 |
| 4062 | UGCAGCCG G UACCUUAG | 3556 | CTAAGGTA GGCTAGCTACAACGA CGGCTGCA | 8353 |
| 4060 | CAGCCGGU A CCUUAGUG | 3557 | CACTAAGG GGCTAGCTACAACGA ACCGGCTG | 8354 |
| 4054 | GUACCUUA G UGCUCUUG | 3558 | CAAGAGCA GGCTAGCTACAACGA TAAGGTAC | 8355 |
| 4052 | ACCUUAGU G CUCUUGCC | 3559 | GGCAAGAG GGCTAGCTACAACGA ACTAAGGT | 8356 |
| 4046 | GUGCUCUU G CCGCUGCC | 3560 | GGCAGCGG GGCTAGCTACAACGA AAGAGCAC | 8357 |
| 4043 | CUCUUGCC G CUGCCAGU | 3561 | ACTGGCAG GGCTAGCTACAACGA GGCAAGAG | 8358 |
| 4040 | UUGCCGCU G CCAGUGGG | 3562 | CCCACTGG GGCTAGCTACAACGA AGCGGCAA | 8359 |
| 4036 | CGCUGCCA G UGGGAGCG | 3563 | CGCTCCCA GGCTAGCTACAACGA TGGCAGCG | 8360 |
| 4030 | CAGUGGGA G CGUGUAGG | 3564 | CCTACACG GGCTAGCTACAACGA TCCCACTG | 8361 |
| 4028 | GUGGGAGC G UGUAGGUG | 3565 | CACCTACA GGCTAGCTACAACGA GCTCCCAC | 8362 |
| 4026 | GGGAGCGU G UAGGUGGG | 3566 | CCCACCTA GGCTAGCTACAACGA ACGCTCCC | 8363 |
| 4022 | GCGUGUAG G UGGGCCAC | 3567 | GTGGCCCA GGCTAGCTACAACGA CTACACGC | 8364 |
| 4018 | GUAGGUGG G CCACUUGG | 3568 | CCAAGTGG GGCTAGCTACAACGA CCACCTAC | 8365 |
| 4015 | GGUGGGCC A CUUGGAAU | 3569 | ATTCCAAG GGCTAGCTACAACGA GGCCCACC | 8366 |
| 4008 | CACUUGGA A UGUCUGCG | 3570 | CGCAGACA GGCTAGCTACAACGA TCCAAGTG | 8367 |
| 4006 | CUUGGAAU G UCUGCGGU | 3571 | ACCGCAGA GGCTAGCTACAACGA ATTCCAAG | 8368 |
| 4002 | GAAUGUCU G CGGUACGG | 3572 | CCGTACCG GGCTAGCTACAACGA AGACATTC | 8369 |
| 3999 | UGUCUGCG G UACGGCUG | 3573 | CAGCCGTA GGCTAGCTACAACGA CGCAGACA | 8370 |
| 3997 | UCUGCGGU A CGGCUGGG | 3574 | CCCAGCCG GGCTAGCTACAACGA ACCGCAGA | 8371 |
| 3994 | GCGGUACG G CUGGGGGG | 3575 | CCCCCCAG GGCTAGCTACAACGA CGTACCGC | 8372 |
| 3984 | UGGGGGGG A CGAGUUGU | 3576 | ACAACTCG GGCTAGCTACAACGA CCCCCCCA | 8373 |
| 3980 | GGGGACGA G UUGUCCGU | 3577 | ACGGACAA GGCTAGCTACAACGA TCGTCCCC | 8374 |
| 3977 | GACGAGUU G UCCGUGAA | 3578 | TTCACGGA GGCTAGCTACAACGA AACTCGTC | 8375 |
| 3973 | AGUUGUCC G UGAAGACC | 3579 | GGTCTTCA GGCTAGCTACAACGA GGACAACT | 8376 |
| 3967 | CCGUGAAG A CCGGGGAC | 3580 | GTCCCCGG GGCTAGCTACAACGA CTTCACGG | 8377 |
| 3960 | GACCGGGG A CCGCAUGG | 3581 | CCATGCGG GGCTAGCTACAACGA CCCCGGTC | 8378 |
| 3957 | CGGGGACC G CAUGGUAG | 3582 | CTACCATG GGCTAGCTACAACGA GGTCCCCG | 8379 |
| 3955 | GGGACCGC A UGGUAGUU | 3583 | AACTACCA GGCTAGCTACAACGA GCGGTCCC | 8380 |
| 3952 | ACCGCAUG G UAGUUUCC | 3584 | GGAAACTA GGCTAGCTACAACGA CATGCGGT | 8381 |
| 3949 | GCAUGGUA G UUUCCAUA | 3585 | TATGGAAA GGCTAGCTACAACGA TACCATGC | 8382 |
| 3943 | UAGUUUCC A UAGACUCA | 3586 | TGAGTCTA GGCTAGCTACAACGA GGAAACTA | 8383 |
| 3939 | UUCCAUAG A CUCAACGG | 3587 | CCGTTGAG GGCTAGCTACAACGA CTATGGAA | 8384 |
| 3934 | UAGACUCA A CGGGUACA | 3588 | TGTACCCG GGCTAGCTACAACGA TGAGTCTA | 8385 |
| 3930 | CUCAACGG G UACAAAGU | 3589 | ACTTTGTA GGCTAGCTACAACGA CCGTTGAG | 8386 |
| 3928 | CAACGGGU A CAAAGUCC | 3590 | GGACTTTG GGCTAGCTACAACGA ACCCGTTG | 8387 |
| 3923 | GGUACAAA G UCCACCGC | 3591 | GCGGTGGA GGCTAGCTACAACGA TTTGTACC | 8388 |
| 3919 | CAAAGUCC A CCGCCUUC | 3592 | GAAGGCGG GGCTAGCTACAACGA GGACTTTG | 8389 |
| 3916 | AGUCCACC G CCUUCGCA | 3593 | TGCGAAGG GGCTAGCTACAACGA GGTGGACT | 8390 |
| 3910 | CCGCCUUC G CAACCCCC | 3594 | GGGGGTTG GGCTAGCTACAACGA GAAGGCGG | 8391 |
| 3907 | CCUUCGCA A CCCCCCGG | 3595 | CCGGGGGG GGCTAGCTACAACGA TGCGAAGG | 8392 |
| 3898 | CCCCCCGG G UGCACACA | 3596 | TGTGTGCA GGCTAGCTACAACGA CCGGGGGG | 8393 |
| 3896 | CCCCGGGU G CACACAGC | 3597 | GCTGTGTG GGCTAGCTACAACGA ACCCGGGG | 8394 |
| 3894 | CCGGGUGC A CACAGCAG | 3598 | CTGCTGTG GGCTAGCTACAACGA GCACCCGG | 8395 |
| 3892 | GGGUGCAC A CAGCAGCC | 3599 | GGCTGCTG GGCTAGCTACAACGA GTGCACCC | 8396 |
| 3889 | UGCACACA G CAGCCCGG | 3600 | CCGGGCTG GGCTAGCTACAACGA TGTGTGCA | 8397 |
| 3886 | ACACAGCA G CCCGGAAG | 3601 | CTTCCGGG GGCTAGCTACAACGA TGCTGTGT | 8398 |
| 3877 | CCCGGAAG A UGCCCACA | 3602 | TGTGGGCA GGCTAGCTACAACGA CTTCCGGG | 8399 |
| 3875 | CGGAAGAU G CCCACAAC | 3603 | GTTGTGGG GGCTAGCTACAACGA ATCTTCCG | 8400 |
| 3871 | AGAUGCCC A CAACGUGC | 3604 | GCACGTTG GGCTAGCTACAACGA GGGCATCT | 8401 |
| 3868 | UGCCCACA A CGUGCCCC | 3605 | GGGGCACG GGCTAGCTACAACGA TGTGGGCA | 8402 |
| 3866 | CCCACAAC G UGCCCCGA | 3606 | TCGGGGCA GGCTAGCTACAACGA GTTGTGGG | 8403 |
| 3864 | CACAACGU G CCCCGAAG | 3607 | CTTCGGGG GGCTAGCTACAACGA ACGTTGTG | 8404 |
| 3854 | CCCGAAGG G CAGAGCAG | 3608 | CTGCTCTG GGCTAGCTACAACGA CCTTCGGG | 8405 |
| 3849 | AGGGCAGA G CAGUGGAC | 3609 | GTCCACTG GGCTAGCTACAACGA TCTGCCCT | 8406 |
| 3846 | GCAGAGCA G UGGACCGC | 3610 | GCGGTCCA GGCTAGCTACAACGA TGCTCTGC | 8407 |
| 3842 | AGCAGUGG A CCGCCCGA | 3611 | TCGGGCGG GGCTAGCTACAACGA CCACTGCT | 8408 |
| 3839 | AGUGGACC G CCCGAGGA | 3612 | TCCTCGGG GGCTAGCTACAACGA GGTCCACT | 8409 |
| 3830 | CCCGAGGA G CCCUUCAA | 3613 | TTGAAGGG GGCTAGCTACAACGA TCCTCGGG | 8410 |
| 3821 | CCCUUCAA G UAGGAGAU | 3614 | ATCTCCTA GGCTAGCTACAACGA TTGAAGGG | 8411 |
| 3814 | AGUAGGAG A UGGGCCUG | 3615 | CAGGCCCA GGCTAGCTACAACGA CTCCTACT | 8412 |
| 3810 | GGAGAUGG G CCUGGGGG | 3616 | CCCCCAGG GGCTAGCTACAACGA CCATCTCC | 8413 |
| 3801 | CCUGGGGG A UAGUAAGC | 3617 | GCTTACTA GGCTAGCTACAACGA CCCCCAGG | 8414 |
| 3798 | GGGGGAUA G UAAGCUCC | 3618 | GGAGCTTA GGCTAGCTACAACGA TATCCCCC | 8415 |
| 3794 | GAUAGUAA G CUCCCCCU | 3619 | AGGGGGAG GGCTAGCTACAACGA TTACTATC | 8416 |
| 3785 | CUCCCCCU G CUGUCACC | 3620 | GGTGACAG GGCTAGCTACAACGA AGGGGGAG | 8417 |
| 3782 | CCCCUGCU G UCACCCCG | 3621 | CGGGGTGA GGCTAGCTACAACGA AGCAGGGG | 8418 |
| 3779 | CUGCUGUC A CCCCGCCG | 3622 | CGGCGGGG GGCTAGCTACAACGA GACAGCAG | 8419 |
| 3774 | GUCACCCC G CCGGCGCA | 3623 | TGCGCCGG GGCTAGCTACAACGA GGGGTGAC | 8420 |
| 3770 | CCCCGCCG G CGCACCGG | 3624 | CCGGTGCG GGCTAGCTACAACGA CGGCGGGG | 8421 |
| 3768 | CCGCCGGC G CACCGGAA | 3625 | TTCCGGTG GGCTAGCTACAACGA GCCGGCGG | 8422 |
| 3766 | GCCGGCGC A CCGGAAUG | 3626 | CATTCCGG GGCTAGCTACAACGA GCGCCGGC | 8423 |
| 3760 | GCACCGGA A UGACAUCA | 3627 | TGATGTCA GGCTAGCTACAACGA TCCGGTGC | 8424 |
| 3757 | CCGGAAUG A CAUCAGCG | 3628 | CGCTGATG GGCTAGCTACAACGA CATTCCGG | 8425 |
| 3755 | GGAAUGAC A UCAGCGUG | 3629 | CACGCTGA GGCTAGCTACAACGA GTCATTCC | 8426 |
| 3751 | UGACAUCA G CGUGUCUC | 3630 | GAGACACG GGCTAGCTACAACGA TGATGTCA | 8427 |
| 3749 | ACAUCAGC G UGUCUCGU | 3631 | ACGAGACA GGCTAGCTACAACGA GCTGATGT | 8428 |
| 3747 | AUCAGCGU G UCUCGUGA | 3632 | TCACGAGA GGCTAGCTACAACGA ACGCTGAT | 8429 |
| 3742 | CGUGUCUC G UGACCAAG | 3633 | CTTGGTCA GGCTAGCTACAACGA GAGACACG | 8430 |
| 3739 | GUCUCGUG A CCAAGUAA | 3634 | TTACTTGG GGCTAGCTACAACGA CACGAGAC | 8431 |
| 3734 | GUGACCAA G UAAAGGUC | 3635 | GACCTTTA GGCTAGCTACAACGA TTGGTCAC | 8432 |
| 3728 | AAGUAAAG G UCCGAGCC | 3636 | GGCTCGGA GGCTAGCTACAACGA CTTTACTT | 8433 |
| 3722 | AGGUCCGA G CCGCCGCA | 3637 | TGCGGCGG GGCTAGCTACAACGA TCGGACCT | 8434 |
| 3719 | UCCGAGCC G CCGCAGGU | 3638 | ACCTGCGG GGCTAGCTACAACGA GGCTCGGA | 8435 |
| 3716 | GAGCCGCC G CAGGUGCA | 3639 | TGCACCTG GGCTAGCTACAACGA GGCGGCTC | 8436 |
| 3712 | CGCCGCAG G UGCAUGGU | 3640 | ACCATGCA GGCTAGCTACAACGA CTGCGGCG | 8437 |
| 3710 | CCGCAGGU G CAUGGUGU | 3641 | ACACCATG GGCTAGCTACAACGA ACCTGCGG | 8438 |
| 3708 | GCAGGUGC A UGGUGUCA | 3642 | TGACACCA GGCTAGCTACAACGA GCACCTGC | 8439 |
| 3705 | UGCAUGGU G UCAAGGAC | 3643 | CCTTGACA GGCTAGCTACAACGA CATGCACC | 8440 |
| 3703 | UGCAUGGU G UCAAGGAC | 3644 | GTCCTTGA GGCTAGCTACAACGA ACCATGCA | 8441 |
| 3696 | UGUCAAGG A CCGCGCUC | 3645 | GAGCGCGG GGCTAGCTACAACGA CCTTGACA | 8442 |
| 3693 | CAAGGACC G CGCUCCGG | 3646 | CCGGAGCG GGCTAGCTACAACGA GGTCCTTG | 8443 |
| 3691 | AGGACCGC G CUCCGGGG | 3647 | CCCCGGAG GGCTAGCTACAACGA GCGGTCCT | 8444 |
| 3681 | UCCGGGGG G CGCCGGCC | 3648 | GGCCGGCG GGCTAGCTACAACGA CCCCCGGA | 8445 |
| 3679 | CGGGGGGC G CCGGCCAU | 3649 | ATGGCCGG GGCTAGCTACAACGA GCCCCCCG | 8446 |
| 3675 | GGGCGCCG G CCAUCCGA | 3650 | TCGGATGG GGCTAGCTACAACGA CGGCGCCC | 8447 |
| 3672 | CGCCGGCC A UCCGACGA | 3651 | TCGTCGGA GGCTAGCTACAACGA GGCCGGCG | 8448 |
| 3667 | GCCAUCCG A CGAGGUCC | 3652 | GGACCTCG GGCTAGCTACAACGA CGGATGGC | 8449 |
| 3662 | CCGACGAG G UCCUGGUC | 3653 | GACCAGGA GGCTAGCTACAACGA CTCGTCGG | 8450 |
| 3656 | AGGUCCUG G UCUACAUU | 3654 | AATGTAGA GGCTAGCTACAACGA CAGGACCT | 8451 |
| 3652 | CCUGGUCU A CAUUGGUG | 3655 | CACCAATG GGCTAGCTACAACGA AGACCAGG | 8452 |
| 3650 | UGGUCUAC A UUGGUGUA | 3656 | TACACCAA GGCTAGCTACAACGA GTAGACCA | 8453 |
| 3646 | CUACAUUG G UGUACAUU | 3657 | AATGTACA GGCTAGCTACAACGA CAATGTAG | 8454 |
| 3644 | ACAUUGGU G UACAUUUG | 3658 | CAAATGTA GGCTAGCTACAACGA ACCAATGT | 8455 |
| 3642 | AUUGGUGU A CAUUUGGG | 3659 | CCCAAATG GGCTAGCTACAACGA ACACCAAT | 8456 |
| 3640 | UGGUGUAC A UUUGGGUG | 3660 | CACCCAAA GGCTAGCTACAACGA GTACACCA | 8457 |
| 3634 | ACAUUUGG G UGAUUGGA | 3661 | TCCAATCA GGCTAGCTACAACGA CCAAATGT | 8458 |
| 3631 | UUUGGGUG A UUGGACCC | 3662 | GGGTCCAA GGCTAGCTACAACGA CACCCAAA | 8459 |
| 3626 | GUGAUUGG A CCCUUUGG | 3663 | CCAAAGGG GGCTAGCTACAACGA CCAATCAC | 8460 |
| 3617 | CCCUUUGG G CCGGCUAG | 3664 | CTAGCCGG GGCTAGCTACAACGA CCAAAGGG | 8461 |
| 3613 | UUGGGCCG G CUAGGGUC | 3665 | GACCCTAG GGCTAGCTACAACGA CGGCCCAA | 8462 |
| 3607 | CGGCUAGG G UCUUUGAG | 3666 | CTCAAAGA GGCTAGCTACAACGA CCTAGCCG | 8463 |
| 3599 | GUCUUUGA G CCGGCGCC | 3667 | GGCGCCGG GGCTAGCTACAACGA TCAAAGAC | 8464 |
| 3595 | UUGAGCCG G CGCCGUGG | 3668 | CCACGGCG GGCTAGCTACAACGA CGGCTCAA | 8465 |
| 3593 | GAGCCGGC G CCGUGGUA | 3669 | TACCACGG GGCTAGCTACAACGA GCCGGCTC | 8466 |
| 3590 | CCGGCGCC G UGGUAGAC | 3670 | GTCTACCA GGCTAGCTACAACGA GGCGCCGG | 8467 |
| 3587 | GCGCCGUG G UAGACAGU | 3671 | ACTGTCTA GGCTAGCTACAACGA CACGGCGC | 8468 |
| 3583 | CGUGGUAG A CAGUCCAG | 3672 | CTGGACTG GGCTAGCTACAACGA CTACCACG | 8469 |
| 3580 | GGUAGACA G UCCAGCAC | 3673 | GTGCTGGA GGCTAGCTACAACGA TGTCTACC | 8470 |
| 3575 | ACAGUCCA G CACACGCC | 3674 | GGCGTGTG GGCTAGCTACAACGA TGGACTGT | 8471 |
| 3573 | AGUCCAGC A CACGCCGU | 3675 | ACGGCGTG GGCTAGCTACAACGA GCTGGACT | 8472 |
| 3571 | UCCAGCAC A CGCCGUUG | 3676 | CAACGGCG GGCTAGCTACAACGA GTGCTGGA | 8473 |
| 3569 | CAGCACAC G CCGUUGAC | 3677 | GTCAACGG GGCTAGCTACAACGA GTGTGCTG | 8474 |
| 3566 | CACACGCC G UUGACGCA | 3678 | TGCGTCAA GGCTAGCTACAACGA GGCGTGTG | 8475 |
| 3562 | CGCCGUUG A CGCAGGUC | 3679 | GACCTGCG GGCTAGCTACAACGA CAACGGCG | 8476 |
| 3560 | CCGUUGAC G CAGGUCGC | 3680 | GCGACCTG GGCTAGCTACAACGA GTCAACGG | 8477 |
| 3556 | UGACGCAG G UCGCUAGG | 3681 | CCTAGCGA GGCTAGCTACAACGA CTGCGTCA | 8478 |
| 3553 | CGCAGGUC G CUAGGAAA | 3682 | TTTCCTAG GGCTAGCTACAACGA GACCTGCG | 8479 |
| 3543 | UAGGAAAG A CUGCGUCG | 3683 | CGACGCAG GGCTAGCTACAACGA CTTTCCTA | 8480 |
| 3540 | GAAAGACU G CGUCGCGG | 3684 | CCGCGACG GGCTAGCTACAACGA AGTCTTTC | 8481 |
| 3538 | AAGACUGC G UCGCGGUG | 3685 | CACCGCGA GGCTAGCTACAACGA GCAGTCTT | 8482 |
| 3535 | ACUGCGUC G CGGUGGAA | 3686 | TTCCACCG GGCTAGCTACAACGA GACGCAGT | 8483 |
| 3532 | GCGUCGCG G UGGAAACC | 3687 | GGTTTCCA GGCTAGCTACAACGA CGCGACGC | 8484 |
| 3526 | CGGUGGAA A CCACUUGA | 3688 | TCAAGTGG GGCTAGCTACAACGA TTCCACCG | 8485 |
| 3523 | UGGAAACC A CUUGAACU | 3689 | AGTTCAAG GGCTAGCTACAACGA GGTTTCCA | 8486 |
| 3517 | CCACUUGA A CUUCCCCC | 3690 | GGGGGAAG GGCTAGCTACAACGA TCAAGTGG | 8487 |
| 3505 | CCCCCUCG A CUUGGUUC | 3691 | GAACCAAG GGCTAGCTACAACGA CGAGGGGG | 8488 |
| 3500 | UCGACUUG G UUCUUGUC | 3692 | GACAAGAA GGCTAGCTACAACGA CAAGTCGA | 8489 |
| 3494 | UGGUUCUU G UCCCGGCC | 3693 | GGCCGGGA GGCTAGCTACAACGA AAGAACCA | 8490 |
| 3488 | UUGUCCCG G CCCGUGAG | 3694 | CTCACGGG GGCTAGCTACAACGA CGGGACAA | 8491 |
| 3484 | CCCGGCCC G UGAGGCUG | 3695 | CAGCCTCA GGCTAGCTACAACGA GGGCCGGG | 8492 |
| 3479 | CCCGUGAG G CUGGUGAU | 3696 | ATCACCAG GGCTAGCTACAACGA CTCACGGG | 8493 |
| 3475 | UGAGGCUG G UGAUAAUG | 3697 | CATTATCA GGCTAGCTACAACGA CAGCCTCA | 8494 |
| 3472 | GGCUGGUG A UAAUGCAG | 3698 | CTGCATTA GGCTAGCTACAACGA CACCAGCC | 8495 |
| 3469 | UGGUGAUA A UGCAGCCA | 3699 | TGGCTGCA GGCTAGCTACAACGA TATCACCA | 8496 |
| 3467 | GUGAUAAU G CAGCCAAA | 3700 | TTTGGCTG GGCTAGCTACAACGA ATTATCAC | 8497 |
| 3464 | AUAAUGCA G CCAAACAG | 3701 | CTGTTTGG GGCTAGCTACAACGA TGCATTAT | 8498 |
| 3459 | GCAGCCAA A CAGGCCCC | 3702 | GGGGCCTG GGCTAGCTACAACGA TTGGCTGC | 8499 |
| 3455 | CCAAACAG G CCCCGCGU | 3703 | ACGCGGGG GGCTAGCTACAACGA CTGTTTGG | 8500 |
| 3450 | CAGGCCCC G CGUCUGUU | 3704 | AACAGACG GGCTAGCTACAACGA GGGGCCTG | 8501 |
| 3448 | GGCCCCGC G UCUGUUGG | 3705 | CCAACAGA GGCTAGCTACAACGA GCGGGGCC | 8502 |
| 3444 | CCGCGUCU G UUGGGAGU | 3706 | ACTCCCAA GGCTAGCTACAACGA AGACGCGG | 8503 |
| 3437 | UGUUGGGA G UAGGCCGU | 3707 | ACGGCCTA GGCTAGCTACAACGA TCCCAACA | 8504 |
| 3433 | GGGAGUAG G CCGUAAUG | 3708 | CATTACGG GGCTAGCTACAACGA CTACTCCC | 8505 |
| 3430 | AGUAGGCC G UAAUGGGC | 3709 | GCCCATTA GGCTAGCTACAACGA GGCCTACT | 8508 |
| 3427 | AGGCCGUA A UGGGCGCG | 3710 | CGCGCCCA GGCTAGCTACAACGA TACGGCCT | 8507 |
| 3423 | CGUAAUGG G CGCGAGGA | 3711 | TCCTCGCG GGCTAGCTACAACGA CCATTACG | 8508 |
| 3421 | UAAUGGGC G CGAGGAGU | 3712 | ACTCCTCG GGCTAGCTACAACGA GCCCATTA | 8509 |
| 3414 | CGCGAGGA G UCGCCACC | 3713 | GGTGGCGA GGCTAGCTACAACGA TCCTCGCG | 8510 |
| 3411 | GAGGAGUC G CCACCCCU | 3714 | AGGGGTGG GGCTAGCTACAACGA GACTCCTC | 8511 |
| 3408 | GAGUCGCC A CCCCUGCC | 3715 | GGCAGGGG GGCTAGCTACAACGA GGCGACTC | 8512 |
| 3402 | CCACCCCU G CCCCUCAA | 3716 | TTGAGGGG GGCTAGCTACAACGA AGGGGTGG | 8513 |
| 3392 | CCCUCAAG A CUGUCGGC | 3717 | GCCGACAG GGCTAGCTACAACGA CTTGAGGG | 8514 |
| 3389 | UCAAGACU G UCGGCUGG | 3718 | CCAGCCGA GGCTAGCTACAACGA AGTCTTGA | 8515 |
| 3385 | GACUGUCG G CUGGUCCU | 3719 | AGGACCAG GGCTAGCTACAACGA CGACAGTC | 8516 |
| 3381 | GUCGGCUG G UCCUAGGA | 3720 | TCCTAGGA GGCTAGCTACAACGA CAGCCGAC | 8517 |
| 3372 | UCCUAGGA G UAUCUCCC | 3721 | GGGAGATA GGCTAGCTACAACGA TCCTAGGA | 8518 |
| 3370 | CUAGGAGU A UCUCCCUC | 3722 | GAGGGAGA GGCTAGCTACAACGA ACTCCTAG | 8519 |
| 3352 | CCCUUCGG G CGGAGACA | 3723 | TGTCTCCG GGCTAGCTACAACGA CCGAAGGG | 8520 |
| 3346 | GGGCGGAG A CAGGUAGA | 3724 | TCTACCTG GGCTAGCTACAACGA CTCCGCCC | 8521 |
| 3342 | GGAGACAG G UAGACCCA | 3725 | TGGGTCTA GGCTAGCTACAACGA CTGTCTCC | 8522 |
| 3338 | CCAUAAUG A UGUCCCCA | 3728 | GGACATCA GGCTAGCTACAACGA TATGGGTC | 8523 |
| 3334 | GUAGACCC A UAAUGAUG | 3727 | CATCATTA GGCTAGCTACAACGA GGGTCTAC | 8524 |
| 3331 | GACCCAUA A UGAUGUCC | 3728 | GGACATCA GGCTAGCTACAACGA TATGGGTC | 8525 |
| 3328 | CCAUAAUG A UGUCCCCA | 3729 | TGGGGACA GGCTAGCTACAACGA CATTATGG | 8526 |
| 3326 | AUAAUGAU G UCCCCACA | 3730 | TGTGGGGA GGCTAGCTACAACGA ATCATTAT | 8527 |
| 3320 | AUGUCCCC A CACGCCGC | 3731 | GCGGCGTG GGCTAGCTACAACGA GGGGACAT | 8528 |
| 3318 | GUCCCCAC A CGCCGCGG | 3732 | CCGCGGCG GGCTAGCTACAACGA GTGGGGAC | 8529 |
| 3316 | CCCCACAC G CCGCGGUG | 3733 | CACCGCGG GGCTAGCTACAACGA GTGTGGGG | 8530 |
| 3313 | CACACGCC G CGGUGUCU | 3734 | AGACACCG GGCTAGCTACAACGA GGCGTGTG | 8531 |
| 3310 | ACGCCGCG G UGUCUCCC | 3735 | GGGAGACA GGCTAGCTACAACGA CGCGGCGT | 8532 |
| 3308 | GCCGCGGU G UCUCCCCC | 3736 | GATCATCA GGCTAGCTACAACGA CTGGGGGG | 8533 |
| 3295 | CCCCCCAG G UGAUGAUC | 3737 | GATCATCA GGCTAGCTACAACGA CTGGGGGG | 8534 |
| 3292 | CCCAGGUG A UGAUCUUG | 3738 | CAAGATCA GGCTAGCTACAACGA CACCTGGG | 8535 |
| 3289 | AGGUGAUG A UCUUGAUU | 3739 | AATCAAGA GGCTAGCTACAACGA CATCACCT | 8536 |
| 3283 | UGAUCUUG A UUUCCAUG | 3740 | CATGGAAA GGCTAGCTACAACGA CAAGATCA | 8537 |
| 3277 | UGAUUUCC A UGUCGGAG | 3741 | CTCCGACA GGCTAGCTACAACGA GGAAATCA | 8538 |
| 3275 | AUUUCCAU G UCGGAGAA | 3742 | TTCTCCGA GGCTAGCTACAACGA ATGGAAAT | 8539 |
| 3265 | CGGAGAAG A CGACGGGC | 3743 | GCCCGTCG GGCTAGCTACAACGA CTTCTCCG | 8540 |
| 3262 | AGAAGACG A CGGGCUCG | 3744 | CGAGCCCG GGCTAGCTACAACGA CGTCTTCT | 8541 |
| 3258 | GACGACGG G CUCGACCG | 3745 | CGGTCGAG GGCTAGCTACAACGA CCGTCGTC | 8542 |
| 3253 | CGGGCUCG A CCGCUACC | 3746 | GGTAGCGG GGCTAGCTACAACGA CGAGCCCG | 8543 |
| 3250 | GCUCGACC G CUACCGCC | 3747 | GGCGGTAG GGCTAGCTACAACGA GGTCGAGC | 8544 |
| 3247 | CGACCGCU A CCGCCAGG | 3748 | CCTGGCGG GGCTAGCTACAACGA AGCGGTCG | 8545 |
| 3244 | CCGCUACC G CCAGGUCU | 3749 | AGACCTGG GGCTAGCTACAACGA GGTAGCCG | 8546 |
| 3239 | ACCGCCAG G UCUCGUAG | 3750 | CTACGAGA GGCTAGCTACAACGA CTGGCGGT | 8547 |
| 3234 | CAGGUCUC G UAGACCUG | 3751 | CAGGTCTA GGCTAGCTACAACGA CAGACCTG | 8548 |
| 3230 | UCUCGUAG A CCUGUGUG | 3752 | CACACAGG GGCTAGCTACAACGA CTACGAGA | 8549 |
| 3226 | GUAGACCU G UGUGGGCC | 3753 | GGCCCACA GGCTAGCTACAACGA AGGTCTAC | 8550 |
| 3224 | AGACCUGU G UGGGCCCA | 3754 | TGGGCCCA GGCTAGCTACAACGA ACAGGTCT | 8551 |
| 3220 | CUGUGUGG G CCCAGUCC | 3755 | GGACTGGG GGCTAGCTACAACGA CCACACAG | 8552 |
| 3215 | UGGGCCCA G UCCUGCAG | 3756 | CTGCAGGA GGCTAGCTACAACGA TGGGCCCA | 8553 |
| 3210 | CCAGUCCU G CAGUGGAG | 3757 | CTCCACTG GGCTAGCTACAACGA AGGACTGG | 8554 |
| 3207 | GUCCUGCA G UGGAGUGA | 3758 | TCACTCCA GGCTAGCTACAACGA TGCAGGAC | 8555 |
| 3202 | GCAGUGGA G UGAGGUGG | 3759 | CCACCTCA GGCTAGCTACAACGA TCCACTGC | 8556 |
| 3197 | GGAGUGAG G UGGUCAUA | 3760 | TATGACCA GGCTAGCTACAACGA CTCACTCC | 8557 |
| 3194 | GUGAGGUG G UCAUAGAC | 3761 | GTCTATGA GGCTAGCTACAACGA CACCTCAC | 8558 |
| 3191 | AGGUGGUC A UAGACGGA | 3762 | TCCGTCTA GGCTAGCTACAACGA GACCACCT | 8559 |
| 3187 | GGUCAUAG A CGGACGUA | 3763 | TACGTCCG GGCTAGCTACAACGA CTATGACC | 8560 |
| 3183 | AUAGACGG A CGUACCUU | 3764 | AAGGTACG GGCTAGCTACAACGA CCGTCTAT | 8561 |
| 3181 | AGACGGAC G UACCUUUC | 3765 | GAAAGGTA GGCTAGCTACAACGA GTCCGTCT | 8562 |
| 3179 | ACGGACGU A CCUUUCAA | 3766 | TTGAAAGG GGCTAGCTACAACGA ACGTCCGT | 8563 |
| 3171 | ACCUUUCA A UUCGGCCA | 3767 | TGGCCGAA GGCTAGCTACAACGA TGAAAGGT | 8564 |
| 3166 | UCAAUUCG G CCAACUUC | 3768 | GAAGTTGG GGCTAGCTACAACGA CGAATTGA | 8565 |
| 3162 | UUCGGCCA A CUUCAUGA | 3769 | TCATGAAG GGCTAGCTACAACGA TGGCCGAA | 8566 |
| 3157 | CCAACUUC A UGAAGGCC | 3770 | GGCCTTCA GGCTAGCTACAACGA GAAGTTGG | 8567 |
| 3151 | UCAUGAAG G CCAUUUGG | 3771 | CCAAATGG GGCTAGCTACAACGA CTTCATGA | 8568 |
| 3148 | UGAAGGCC A UUUGGACA | 3772 | TGTCCAAA GGCTAGCTACAACGA GGCCTTCA | 8569 |
| 3142 | CCAUUUGG A CAUAUUGC | 3773 | GCAATATG GGCTAGCTACAACGA CCAAATGG | 8570 |
| 3140 | AUUUGGAC A UAUUGCCC | 3774 | GGGCAATA GGCTAGCTACAACGA GTCCAAAT | 8571 |
| 3138 | UUGGACAU A UUGCCCCC | 3775 | GGGGGCAA GGCTAGCTACAACGA ATGTCCAA | 8572 |
| 3135 | GACAUAUU G CCCCCCAC | 3776 | GTGGGGGG GGCTAGCTACAACGA AATATGTC | 8573 |
| 3128 | UGCCCCCC A CCGACUUU | 3777 | AAAGTCGG GGCTAGCTACAACGA GGGGGGCA | 8574 |
| 3124 | CCCCACCG A CUUUCCGC | 3778 | GCGGAAAG GGCTAGCTACAACGA CGGTGGGG | 8575 |
| 3117 | GACUUUCC G CACCAAAA | 3779 | TTTTGGTG GGCTAGCTACAACGA GGAAAGTC | 8576 |
| 3115 | CUUUCCGC A CCAAAAUG | 3780 | CATTTTGG GGCTAGCTACAACGA GCGGAAAG | 8577 |
| 3109 | GCACCAAA A UGCAUUCA | 3781 | TGAATGCA GGCTAGCTACAACGA TTTGGTGC | 8578 |
| 3107 | ACCAAAAU G CAUUCACG | 3782 | CGTGAATG GGCTAGCTACAACGA ATTTTGGT | 8579 |
| 3105 | CAAAAUGC A UUCACGGA | 3783 | TCCGTGAA GGCTAGCTACAACGA GCATTTTG | 8580 |
| 3101 | AUGCAUUC A CGGAUGAC | 3784 | GTCATCCG GGCTAGCTACAACGA GAATGCAT | 8581 |
| 3097 | AUUCACGG A UGACCCCU | 3785 | AGGGGTCA GGCTAGCTACAACGA CCGTGAAT | 8582 |
| 3094 | CACGGAUG A CCCCUUGA | 3786 | TCAAGGGG GGCTAGCTACAACGA CATCCGTG | 8583 |
| 3085 | CCCCUUGA G CCCGCACA | 3787 | TGTGCGGG GGCTAGCTACAACGA TCAAGGGG | 8584 |
| 3081 | UUGAGCCC G CACAAAGU | 3788 | ACTTTGTG GGCTAGCTACAACGA GGGCTCAA | 8585 |
| 3079 | GAGCCCGC A CAAAGUCC | 3789 | GGACTTTG GGCTAGCTACAACGA GCGGGCTC | 8586 |
| 3074 | CGCACAAA G UCCGGCAC | 3790 | GTGCCGGA GGCTAGCTACAACGA TTTGTGCG | 8587 |
| 3069 | AAAGUCCG G CACUUUUG | 3791 | CAAAAGTG GGCTAGCTACAACGA CGGACTTT | 8588 |
| 3067 | AGUCCGGC A CUUUUGCU | 3792 | AGCAAAAG GGCTAGCTACAACGA GCCGGACT | 8589 |
| 3061 | GCACUUUU G CUAUACCA | 3793 | TGGTATAG GGCTAGCTACAACGA AAAAGTGC | 8590 |
| 3058 | CUUUUGCU A UACCAGCC | 3794 | GGCTGGTA GGCTAGCTACAACGA AGCAAAAG | 8591 |
| 3056 | UUUGCUAU A CCAGCCUG | 3795 | CAGGCTGG GGCTAGCTACAACGA ATAGCAAA | 8592 |
| 3052 | CUAUACCA G CCUGGAGC | 3796 | GCTCCAGG GGCTAGCTACAACGA TGGTATAG | 8593 |
| 3045 | AGCCUGGA G CACCAUGA | 3797 | TCATGGTG GGCTAGCTACAACGA TCCAGGCT | 8594 |
| 3043 | CCUGGAGC A CCAUGAGC | 3798 | GCTCATGG GGCTAGCTACAACGA GCTCCAGG | 8595 |
| 3040 | GGAGCACC A UGAGCGGG | 3799 | CCCGCTCA GGCTAGCTACAACGA GGTGCTCC | 8596 |
| 3036 | CACCAUGA G CGGGCCGA | 3800 | TCGGCCCG GGCTAGCTACAACGA TCATGGTG | 8597 |
| 3032 | AUGAGCGG G CCGAGUAU | 3801 | ATACTCGG GGCTAGCTACAACGA CCGCTCAT | 8598 |
| 3027 | CGGGCCGA G UAUGGCGA | 3802 | TCGCCATA GGCTAGCTACAACGA TCGGCCCG | 8599 |
| 3025 | GGCCGAGU A UGGCGAGC | 3803 | GCTCGCCA GGCTAGCTACAACGA ACTCGGCC | 8600 |
| 3022 | CGAGUAUG G CGAGCAUA | 3804 | TATGCTCG GGCTAGCTACAACGA CATACTCG | 8601 |
| 3018 | UAUGGCGA G CAUAAUUU | 3805 | AAATTATG GGCTAGCTACAACGA TCGCCATA | 8602 |
| 3016 | UGGCGAGC A UAAUUUUG | 3806 | CAAAATTA GGCTAGCTACAACGA GCTCGCCA | 8603 |
| 3013 | CGAGCAUA A UUUUGGUG | 3807 | CACCAAAA GGCTAGCTACAACGA TATGCTCG | 8604 |
| 3007 | UAAUUUUG G UGAUGUCA | 3808 | TGACATCA GGCTAGCTACAACGA CAAAATTA | 8605 |
| 3004 | UUUUGGUG A UGUCAAAG | 3809 | CTTTGACA GGCTAGCTACAACGA CACCAAAA | 8606 |
| 3002 | UUGGUGAU G UCAAAGAU | 3810 | ATCTTTGA GGCTAGCTACAACGA ATCACCAA | 8607 |
| 2995 | UGUCAAAG A UUAGCUCU | 3811 | AGAGCTAA GGCTAGCTACAACGA CTTTGACA | 8608 |
| 2991 | AAAGAUUA G CUCUGGGU | 3812 | ACCCAGAG GGCTAGCTACAACGA TAATCTTT | 8609 |
| 2984 | AGCUCUGG G UGGACCAC | 3813 | GTGGTCCA GGCTAGCTACAACGA CCAGAGCT | 8610 |
| 2980 | CUGGGUGG A CCACACAC | 3814 | GTGTGTGG GGCTAGCTACAACGA CCACCCAG | 8611 |
| 2977 | GGUGGACC A CACACGUG | 3815 | CACGTGTG GGCTAGCTACAACGA GGTCCACC | 8612 |
| 2975 | UGGACCAC A CACGUGAG | 3816 | CTCACGTG GGCTAGCTACAACGA GTGGTCCA | 8613 |
| 2973 | GACCACAC A CGUGAGGA | 3817 | TCCTCACG GGCTAGCTACAACGA GTGTGGTC | 8614 |
| 2971 | CCACACAC G UGAGGAGA | 3818 | TCTCCTCA GGCTAGCTACAACGA GTGTGTGG | 8615 |
| 2962 | UGAGGAGA A UGAUGGCA | 3819 | TGCCATCA GGCTAGCTACAACGA TCTCCTCA | 8616 |
| 2959 | GGAGAAUG A UGGCACCG | 3820 | CGGTGCCA GGCTAGCTACAACGA CATTCTCC | 8617 |
| 2956 | GAAUGAUG G CACCGCGC | 3821 | GCGCGGTG GGCTAGCTACAACGA CATCATTC | 8618 |
| 2954 | AUGAUGGC A CCGCGCCC | 3822 | GGGCGCGG GGCTAGCTACAACGA GCCATCAT | 8619 |
| 2951 | AUGGCACC G CGCCCCCC | 3823 | GGGGGGCG GGCTAGCTACAACGA GGTGCCAT | 8620 |
| 2949 | GGCACCGC G CCCCCCCC | 3824 | GGGGGGGG GGCTAGCTACAACGA GCGGTGCC | 8621 |
| 2938 | CCCCCCGA A CGUUGAGG | 3825 | CCTCAACG GGCTAGCTACAACGA TCGGGGGG | 8622 |
| 2936 | CCCCGAAC G UUGAGGGG | 3826 | CCCCTCAA GGCTAGCTACAACGA GTTCGGGG | 8623 |
| 2923 | GGGGGGGG A UCCACACU | 3827 | AGTGTGGA GGCTAGCTACAACGA CCCCCCCC | 8624 |
| 2919 | GGGGAUCC A CACUUGCA | 3828 | TGCAAGTG GGCTAGCTACAACGA GGATCCCC | 8625 |
| 2917 | GGAUCCAC A CUUGCAAC | 3829 | GTTGCAAG GGCTAGCTACAACGA GTGGATCC | 8626 |
| 2913 | CCACACUU G CAACUGCG | 3830 | CGCAGTTG GGCTAGCTACAACGA AAGTGTGG | 8627 |
| 2910 | CACUUGCA A CUGCGCCU | 3831 | AGGCGCAG GGCTAGCTACAACGA TGCAAGTG | 8628 |
| 2907 | UUGCAACU G CGCCUCGG | 3832 | CCGAGGCG GGCTAGCTACAACGA AGTTGCAA | 8629 |
| 2905 | GCAACUGC G CCUCGGCU | 3833 | AGCCGAGG GGCTAGCTACAACGA CGAGGCGC | 8630 |
| 2899 | GCGCCUCG G CUCUGGUG | 3834 | CACCAGAG GGCTAGCTACAACGA CGAGGCGC | 8631 |
| 2893 | CGGCUCUG G UGAUAAGG | 3835 | CCTTATCA GGCTAGCTACAACGA CAGAGCCG | 8632 |
| 2890 | CUCUGGUG A UAAGGUAU | 3836 | ATACCTTA GGCTAGCTACAACGA CACCAGAG | 8633 |
| 2885 | GUGAUAAG G UAUUGCAA | 3837 | TTGCAATA GGCTAGCTACAACGA CTTATCAC | 8634 |
| 2883 | GAUAAGGU A UUGCAACC | 3838 | GGTTGCAA GGCTAGCTACAACGA ACCTTATC | 8635 |
| 2880 | AAGGUAUU G CAACCACC | 3839 | GGTGGTTG GGCTAGCTACAACGA AATACCTT | 8636 |
| 2877 | GUAUUGCA A CCACCAUA | 3840 | TATGGTGG GGCTAGCTACAACGA TGCAATAC | 8637 |
| 2874 | UUGCAACC A CCAUAUGA | 3841 | TCATATGG GGCTAGCTACAACGA GGTTGCAA | 8638 |
| 2871 | CAACCACC A UAUGAGCC | 3842 | GGCTCATA GGCTAGCTACAACGA GGTGGTTG | 8639 |
| 2869 | ACCACCAU A UGAGCCUA | 3843 | TAGGCTCA GGCTAGCTACAACGA ATGGTGGT | 8640 |
| 2865 | CCAUAUGA G CCUAGCGA | 3844 | TCGCTAGG GGCTAGCTACAACGA TCATATGG | 8641 |
| 2860 | UGAGCCUA G CGAGGAAC | 3845 | GTTCCTCG GGCTAGCTACAACGA TAGGCTCA | 8642 |
| 2853 | AGCGAGGA A CACUUUGU | 3846 | ACAAAGTG GGCTAGCTACAACGA TCCTCGCT | 8643 |
| 2851 | CGAGGAAC A CUUUGUAG | 3847 | CTACAAAG GGCTAGCTACAACGA GTTCCTCG | 8644 |
| 2846 | AACACUUU G UAGUAUGG | 3848 | CCATACTA GGCTAGCTACAACGA AAAGTGTT | 8645 |
| 2843 | ACUUUGUA G UAUGGUGA | 3849 | TCACCATA GGCTAGCTACAACGA TACAAAGT | 8646 |
| 2841 | UUUGUAGU A UGGUGACA | 3850 | TGTCACCA GGCTAGCTACAACGA ACTACAAA | 8647 |
| 2838 | GUAGUAUG G UGACAAGG | 3851 | CCTTGTCA GGCTAGCTACAACGA CATACTAC | 8648 |
| 2835 | GUAUGGUG A CAAGGUCA | 3852 | TGACCTTG GGCTAGCTACAACGA CACCATAC | 8649 |
| 2830 | GUGACAAG G UCAAGAGU | 3853 | ACTCTTGA GGCTAGCTACAACGA CTTGTCAC | 8650 |
| 2823 | GGUCAAGA G UGCUAGAC | 3854 | GTCTAGCA GGCTAGCTACAACGA TCTTGACC | 8651 |
| 2821 | UCAAGAGU G CUAGACCU | 3855 | AGGTCTAG GGCTAGCTACAACGA ACTCTTGA | 8652 |
| 2816 | AGUGCUAG A CCUACAAA | 3856 | TTTGTAGG GGCTAGCTACAACGA CTAGCACT | 8653 |
| 2812 | CUAGACCU A CAAAAACC | 3857 | GGTTTTTG GGCTAGCTACAACGA AGGTCTAG | 8654 |
| 2806 | CUACAAAA A CCACGCCU | 3858 | AGGCGTGG GGCTAGCTACAACGA TTTTGTAG | 8655 |
| 2803 | CAAAAACC A CGCCUCCG | 3859 | CGGAGGCG GGCTAGCTACAACGA GGTTTTTG | 8656 |
| 2801 | AAAACCAC G CCUCCGCA | 3860 | TGCGGAGG GGCTAGCTACAACGA GTGGTTTT | 8657 |
| 2795 | ACGCCUCC G CACGAUGC | 3861 | GCATCGTG GGCTAGCTACAACGA GGAGGCGT | 8658 |
| 2793 | GCCUCCGC A CGAUGCGG | 3862 | CCGCATCG GGCTAGCTACAACGA GCGGAGGC | 8659 |
| 2790 | UCCGCACG A UGCGGCCA | 3863 | TGGCCGCA GGCTAGCTACAACGA CGTGCGGA | 8660 |
| 2788 | CGCACGAU G CGGCCAUC | 3864 | GATGGCCG GGCTAGCTACAACGA ATCGTGCG | 8661 |
| 2785 | ACGAUGCG G CCAUCUCC | 3865 | GGAGATGG GGCTAGCTACAACGA CGCATCGT | 8662 |
| 2782 | AUGCGGCC A UCUCCCGG | 3866 | CCGGGAGA GGCTAGCTACAACGA GGCCGCAT | 8663 |
| 2774 | AUCUCCCG G UCCAUGGC | 3867 | GCCATGGA GGCTAGCTACAACGA CGGGAGAT | 8664 |
| 2770 | CCCGGUCC A UGGCGUAC | 3868 | GTACGCCA GGCTAGCTACAACGA GGACCGGG | 8665 |
| 2767 | GGUCCAUG G CGUACGCC | 3869 | GGCGTACG GGCTAGCTACAACGA CATGGACC | 8666 |
| 2765 | UCCAUGGC G UACGCCCG | 3870 | CGGGCGTA GGCTAGCTACAACGA GCCATGGA | 8667 |
| 2763 | CAUGGCGU A CGCCCGUG | 3871 | CACGGGCG GGCTAGCTACAACGA ACGCCATG | 8668 |
| 2761 | UGGCGUAC G CCCGUGGU | 3872 | ACCACGGG GGCTAGCTACAACGA GTACGCCA | 8669 |
| 2757 | GUACGCCC G UGGUGGUA | 3873 | TACCACCA GGCTAGCTACAACGA GGGCGTAC | 8670 |
| 2754 | CGCCCGUG G UGGUAACG | 3874 | CGTTACCA GGCTAGCTACAACGA CACGGGCG | 8671 |
| 2751 | CCGUGGUG G UAACGCCA | 3875 | TGGCGTTA GGCTAGCTACAACGA CACCACGG | 8672 |
| 2748 | UGGUGGUA A CGCCAGCA | 3876 | TGCTGGCG GGCTAGCTACAACGA TACCACCA | 8673 |
| 2746 | GUGGUAAC G CCAGCAGG | 3877 | CCTGCTGG GGCTAGCTACAACGA GTTACCAC | 8674 |
| 2742 | UAACGCCA G CAGGAGCA | 3878 | TGCTCCTG GGCTAGCTACAACGA TGGCGTTA | 8675 |
| 2736 | CAGCAGGA G CAGGAGUA | 3879 | TACTCCTG GGCTAGCTACAACGA TCCTGCTG | 8676 |
| 2730 | GAGCAGGA G UAGCGGCC | 3880 | GGCCGCTA GGCTAGCTACAACGA TCCTGCTC | 8677 |
| 2727 | CAGGAGUA G CGGCCAUA | 3881 | TATGGCCG GGCTAGCTACAACGA TACTCCTG | 8678 |
| 2724 | GAGUAGCG G CCAUACGC | 3882 | GCGTATGG GGCTAGCTACAACGA CGCTACTC | 8679 |
| 2721 | UAGCGGCC A UACGCCGU | 3883 | ACGGCGTA GGCTAGCTACAACGA GGCCGCTA | 8680 |
| 2719 | GCGGCCAU A CGCCGUAG | 3884 | CTACGGCG GGCTAGCTACAACGA ATGGCCGC | 8681 |
| 2717 | GGCCAUAC G CCGUAGAG | 3885 | CTCTACGG GGCTAGCTACAACGA GTATGGCC | 8682 |
| 2714 | CAUACGCC G UAGAGAGC | 3886 | GCTCTCTA GGCTAGCTACAACGA GGCGTATG | 8683 |
| 2707 | CGUAGAGA G CAUAUGCC | 3887 | GGCATATG GGCTAGCTACAACGA TCTCTACG | 8684 |
| 2705 | UAGAGAGC A UAUGCCGC | 3888 | GCGGCATA GGCTAGCTACAACGA GCTCTCTA | 8685 |
| 2703 | GAGAGCAU A UGCCGCCC | 3889 | GGGCGGCA GGCTAGCTACAACGA ATGCTCTC | 8686 |
| 2701 | GAGCAUAU G CCGCCCCA | 3890 | TGGGGCGG GGCTAGCTACAACGA ATATGCTC | 8687 |
| 2698 | CAUAUGCC G CCCCAGGG | 3891 | CCCTGGGG GGCTAGCTACAACGA GGCATATG | 8688 |
| 2689 | CCCCAGGG A CCAGCUUG | 3892 | CAAGCTGG GGCTAGCTACAACGA CCCTGGGG | 8689 |
| 2685 | AGGGACCA G CUUGCCUU | 3893 | AAGGCAAG GGCTAGCTACAACGA TGGTCCCT | 8690 |
| 2681 | ACCAGCUU G CCUUUGAU | 3894 | ATCAAAGG GGCTAGCTACAACGA AAGCTGGT | 8691 |
| 2674 | UGCCUUUG A UGUACCAG | 3895 | CTGGTACA GGCTAGCTACAACGA CAAAGGCA | 8692 |
| 2672 | CCUUUGAU G UACCAGGC | 3896 | GCCTGGTA GGCTAGCTACAACGA ATCAAAGG | 8693 |
| 2670 | UUUGAUGU A CCAGGCAG | 3897 | CTGCCTGG GGCTAGCTACAACGA ACATCAAA | 8694 |
| 2665 | UGUACCAG G CAGCACAG | 3898 | CTGTGCTG GGCTAGCTACAACGA CTGGTACA | 8695 |
| 2662 | ACCAGGCA G CACAGAAG | 3899 | CTTCTGTG GGCTAGCTACAACGA TGCCTGGT | 8696 |
| 2660 | CAGGCAGC A CAGAAGAA | 3900 | TTCTTCTG GGCTAGCTACAACGA GCTGCCTG | 8697 |
| 2652 | ACAGAAGA A CACGAGGA | 3901 | TCCTCGTG GGCTAGCTACAACGA TCTTCTGT | 8698 |
| 2650 | AGAAGAAC A CGAGGAAG | 3902 | CTTCCTCG GGCTAGCTACAACGA GTTCTTCT | 8699 |
| 2635 | AGGAGAGG A UGCCAUGC | 3903 | GCATGGCA GGCTAGCTACAACGA CCTCTCCT | 8700 |
| 2633 | GAGAGGAU G CCAUGCAC | 3904 | GTGCATGG GGCTAGCTACAACGA ATCCTCTC | 8701 |
| 2630 | AGGAUGCC A UGCACUCC | 3905 | GGAGTGCA GGCTAGCTACAACGA GGCATCCT | 8702 |
| 2628 | GAUGCCAU G CACUCCGG | 3906 | CCGGAGTG GGCTAGCTACAACGA ATGGCATC | 8703 |
| 2626 | UGCCAUGC A CUCCGGCC | 3907 | GGCCGGAG GGCTAGCTACAACGA GCATGGCA | 8704 |
| 2620 | GCAGUCCG G CCAAGGAU | 3908 | ATCCTTGG GGCTAGCTACAACGA CGGAGTGC | 8705 |
| 2613 | GGCCAAGG A UGCUGCAU | 3909 | ATGCAGCA GGCTAGCTACAACGA CCTTGGCC | 8706 |
| 2611 | CCAAGGAU G CUGCAUUG | 3910 | CAATGCAG GGCTAGCTACAACGA ATCCTTGG | 8707 |
| 2608 | AGGAUGCU G CAUUGAGG | 3911 | CCTCAATG GGCTAGCTACAACGA AGCATCCT | 8708 |
| 2606 | GAUGCUGC A UUGAGGAC | 3912 | GTCCTCAA GGCTAGCTACAACGA GCAGCATC | 8709 |
| 2599 | CAUUGAGG A CCACCAGG | 3913 | CCTGGTGG GGCTAGCTACAACGA CCTCAATG | 8710 |
| 2596 | UGAGGACC A CCAGGUUC | 3914 | GAACCTGG GGCTAGCTACAACGA GGTCCTCA | 8711 |
| 2591 | ACCACCAG G UUCUCUAG | 3915 | CTAGAGAA GGCTAGCTACAACGA CTGGTGGT | 8712 |
| 2581 | UCUCUAGG G CAGCCUCG | 3916 | CGAGGCTG GGCTAGCTACAACGA CCTAGAGA | 8713 |
| 2578 | CUAGGGCA G CCUCGGCC | 3917 | GGCCGAGG GGCTAGCTACAACGA TGCCCTAG | 8714 |
| 2572 | CAGCCUCG G CCUGGGCU | 3918 | AGCCCAGG GGCTAGCTACAACGA CGAGGCTG | 8715 |
| 2566 | CGGCCUGG G CUACCAAC | 3919 | GTTGGTAG GGCTAGCTACAACGA CCAGGCCG | 8716 |
| 2563 | CCUGGGCU A CCAACAGC | 3920 | GCTGTTGG GGCTAGCTACAACGA AGCCCAGG | 8717 |
| 2559 | GGCUACCA A CAGCAUCA | 3921 | TGATGCTG GGCTAGCTACAACGA TGGTAGCC | 8718 |
| 2556 | UACCAACA G CAUCAUCC | 3922 | GGATGATG GGCTAGCTACAACGA TGTTGGTA | 8719 |
| 2554 | CCAACAGC A UCAUCCAC | 3923 | GTGGATGA GGCTAGCTACAACGA GCTGTTGG | 8720 |
| 2551 | ACAGCAUC A UCCACAAA | 3924 | TTTGTGGA GGCTAGCTACAACGA GATGCTGT | 8721 |
| 2547 | CAUCAUCC A CAAACAGG | 3925 | CCTGTTTG GGCTAGCTACAACGA GGATGATG | 8722 |
| 2543 | AUCCACAA A CAGGCACA | 3926 | TGTGCCTG GGCTAGCTACAACGA TTGTGGAT | 8723 |
| 2539 | ACAAACAG G CACAGACG | 3927 | CGTCTGTG GGCTAGCTACAACGA CTGTTTGT | 8724 |
| 2537 | AAACAGGC A CAGACGCG | 3928 | CGCGTCTG GGCTAGCTACAACGA GCCTGTTT | 8725 |
| 2533 | AGGCACAG A CGCGCGCG | 3929 | CGCGCGCG GGCTAGCTACAACGA CTGTGCCT | 8726 |
| 2531 | GCACAGAC G CGCGCGUC | 3930 | GACGCGCG GGCTAGCTACAACGA GTCTGTGC | 8727 |
| 2529 | ACAGACGC G CGCGUCUG | 3931 | CAGACGCG GGCTAGCTACAACGA GCGTCTGT | 8728 |
| 2527 | AGACGCGC G CGUCUGCC | 3932 | GGCAGACG GGCTAGCTACAACGA GCGCGTCT | 8729 |
| 2525 | ACGCGCGC G UCUGCCAG | 3933 | CTGGCAGA GGCTAGCTACAACGA GCGCGCGT | 8730 |
| 2521 | GCGCGUCU G CCAGGAGA | 3934 | TCTCCTGG GGCTAGCTACAACGA AGACGCGC | 8731 |
| 2505 | AAGGAAAA G CAACAGGA | 3935 | TCCTGTTG GGCTAGCTACAACGA TTTTCCTT | 8732 |
| 2502 | GAAAAGCA A CAGGACAU | 3936 | ATGTCCTG GGCTAGCTACAACGA TGCTTTTC | 8733 |
| 2497 | GCAACAGG A CAUACUCC | 3937 | GGAGTATG GGCTAGCTACAACGA CCTGTTGC | 8734 |
| 2495 | AACAGGAC A UACUCCCA | 3938 | TGGGAGTA GGCTAGCTACAACGA GTCCTGTT | 8735 |
| 2493 | CAGGACAU A CUCCCAUU | 3939 | AATGGGAG GGCTAGCTACAACGA ATGTCCTG | 8736 |
| 2487 | AUACUCCC A UUUGAUUG | 3940 | CAATCAAA GGCTAGCTACAACGA GGGAGTAT | 8737 |
| 2482 | CCCAUUUG A UUGCGAAG | 3941 | CTTCGCAA GGCTAGCTACAACGA CAAATGGG | 8738 |
| 2479 | AUUUGAUU G CGAAGGAG | 3942 | CTCCTTCG GGCTAGCTACAACGA AATCAAAT | 8739 |
| 2470 | CGAAGGAG A CAACCGCU | 3943 | AGCGGTTG GGCTAGCTACAACGA CTCCTTCG | 8740 |
| 2467 | AGGAGACA A CCGCUGAC | 3944 | GTCAGCGG GGCTAGCTACAACGA TGTCTCCT | 8741 |
| 2464 | AGACAACC G CUGACCCU | 3945 | AGGGTCAG GGCTAGCTACAACGA GGTTGTCT | 8742 |
| 2460 | AACCGCUG A CCCUACAC | 3946 | GTGTAGGG GGCTAGCTACAACGA CAGCGGTT | 8743 |
| 2455 | CUGACCCU A CACCGUAC | 3947 | GTACGGTG GGCTAGCTACAACGA AGGGTCAG | 8744 |
| 2453 | GACCCUAC A CCGUACAG | 3948 | CTGTACGG GGCTAGCTACAACGA GTAGGGTC | 8745 |
| 2450 | CCUACACC G UACAGGUA | 3949 | TACCTGTA GGCTAGCTACAACGA GGTGTAAG | 8746 |
| 2448 | UACACCGU A CAGGUAUU | 3950 | AATACCTG GGCTAGCTACAACGA ACGGTGTA | 8747 |
| 2444 | CCGUACAG G UAUUGCAC | 3951 | GTGCAATA GGCTAGCTACAACGA CTGTACGG | 8748 |
| 2442 | GUACAGGU A UUGCACGU | 3952 | ACGTGCAA GGCTAGCTACAACGA ACCTGTAC | 8749 |
| 2439 | CAGGUAUU G CACGUCCA | 3953 | TGGACGTG GGCTAGCTACAACGA AATACCTG | 8750 |
| 2437 | GGUAUUGC A CGUCCACG | 3954 | CGTGGACG GGCTAGCTACAACGA GCAATACC | 8751 |
| 2435 | UAUUGCAC G UCCACGAU | 3955 | ATCGTGGA GGCTAGCTACAACGA GTGCAATA | 8752 |
| 2431 | GCACGUCC A CGAUGUUC | 3956 | GAACATCG GGCTAGCTACAACGA GGACGTGC | 8753 |
| 2428 | CGUCCACG A UGUUCUGG | 3957 | CCAGAACA GGCTAGCTACAACGA CGTGGACG | 8754 |
| 2426 | UCCACGAU G UUCUGGUG | 3958 | CACCAGAA GGCTAGCTACAACGA ATCGTGGA | 8755 |
| 2420 | AUGUUCUG G UGGAGAUG | 3959 | CATCTCCA GGCTAGCTACAACGA CAGAACAT | 8756 |
| 2414 | UGGUGGAG A UGGAUCAA | 3960 | TTGATCCA GGCTAGCTACAACGA CTCCACCA | 8757 |
| 2410 | GGAGAUGG A UCAAACCA | 3961 | TGGTTTGA GGCTAGCTACAACGA CCATCTCC | 8758 |
| 2405 | UGGAUCAA A CCAGUGGA | 3962 | TCCACTGG GGCTAGCTACAACGA TTGATCCA | 8759 |
| 2401 | UCAAACCA G UGGACAGA | 3963 | TCTGTCCA GGCTAGCTACAACGA TGGTTTGA | 8760 |
| 2397 | ACCAGUGG A CAGAGCCG | 3964 | CGGCTCTG GGCTAGCTACAACGA CCACTGGT | 8761 |
| 2392 | UGGACAGA G CCGGUAGG | 3965 | CCTACCGG GGCTAGCTACAACGA TCTGTCCA | 8762 |
| 2388 | CAGAGCCG G UAGGGUGG | 3966 | CCACCCTA GGCTAGCTACAACGA CGGCTCTG | 8763 |
| 2383 | CCGGUAGG G UGGUGAAG | 3967 | CTTCACCA GGCTAGCTACAACGA CCTACCGG | 8764 |
| 2380 | GUAGGGUG G UGAAGGAG | 3968 | CTCCTTCA GGCTAGCTACAACGA CACCCTAC | 8765 |
| 2372 | GUGAAGGA G CAGGGCAG | 3969 | CTGCCCTG GGCTAGCTACAACGA TCCTTCAC | 8766 |
| 2367 | GGAGCAGG G CAGUAUUU | 3970 | AAATACTG GGCTAGCTACAACGA CCTGCTCC | 8767 |
| 2364 | GCAGGGCA G UAUUUGCC | 3971 | GGCAAATA GGCTAGCTACAACGA TGCCCTGC | 8768 |
| 2362 | AGGGCAGU A UUUGCCAC | 3972 | GTGGCAAA GGCTAGCTACAACGA ACTGCCCT | 8769 |
| 2358 | CAGUAUUU G CCACUCUG | 3973 | CAGAGTGG GGCTAGCTACAACGA AAATACTG | 8770 |
| 2355 | UAUUUGCC A CUCUGUAG | 3974 | CTACAGAG GGCTAGCTACAACGA GGCAAATA | 8771 |
| 2350 | GCCACUCU G UAGUGGAC | 3975 | GTCCACTA GGCTAGCTACAACGA AGAGTGGC | 8772 |
| 2347 | ACUCUGUA G UGGACAAC | 3976 | GTTGTCCA GGCTAGCTACAACGA TACAGAGT | 8773 |
| 2343 | UGUAGUGG A CAACAGCA | 3977 | TGCTGTTG GGCTAGCTACAACGA CCACTACA | 8774 |
| 2340 | AGUGGACA A CAGCAGCG | 3978 | CGCTGCTG GGCTAGCTACAACGA TGTCCACT | 8775 |
| 2337 | GGACAACA G CAGCGGGC | 3979 | GCCCGCTG GGCTAGCTACAACGA TGTTGTCC | 8776 |
| 2334 | CAACAGCA G CGGGCUGA | 3980 | TCAGCCCG GGCTAGCTACAACGA TGCTGTTG | 8777 |
| 2330 | AGCAGCGG G CUGAGCUC | 3981 | GAGCTCAG GGCTAGCTACAACGA CCGCTGCT | 8778 |
| 2325 | CGGGCUGA G CUCUGAUC | 3982 | GGGACAGA GGCTAGCTACAACGA TCAGCCCG | 8779 |
| 2319 | GAGCUCUG A UCUGUCCC | 3983 | GGGACAGA GGCTAGCTACAACGA CAGAGCTC | 8780 |
| 2315 | UCUGAUCU G UCCCUGUC | 3984 | GACAGGGA GGCTAGCTACAACGA AGATCAGA | 8781 |
| 2309 | CUGUCCCU G UCCUCCAA | 3985 | TTGGAGGA GGCTAGCTACAACGA AGGGACAG | 8782 |
| 2300 | UCCUCCAA A UCACAACG | 3986 | CGTTGTGA GGCTAGCTACAACGA TTGGAGGA | 8783 |
| 2297 | UCCAAAUC A CAACGCUC | 3987 | GAGCGTTG GGCTAGCTACAACGA GATTTGGA | 8784 |
| 2294 | AAAUCACA A CGCUCUCC | 3988 | GGAGAGCG GGCTAGCTACAACGA TGTGATTT | 8785 |
| 2292 | AUCACAAC G CUCUCCUC | 3989 | GAGGAGAG GGCTAGCTACAACGA GTTGTGAT | 8786 |
| 2281 | CUCCUCGA G UCCAAUUG | 3990 | CAATTGGA GGCTAGCTACAACGA TCGAGGAG | 8787 |
| 2276 | CGAGUCCA A UUGCAUGC | 3991 | GCATGCAA GGCTAGCTACAACGA TGGACTCG | 8788 |
| 2273 | GUCCAAUU G CAUGCGGC | 3992 | GCCGCATG GGCTAGCTACAACGA AATTGGAC | 8789 |
| 2271 | CCAAUUGC A UGCGGCGG | 3993 | CCGCCGCA GGCTAGCTACAACGA GCAATTGG | 8790 |
| 2269 | AAUUGCAU G CGGCGGUG | 3994 | CACCGCCG GGCTAGCTACAACGA ATGCAATT | 8791 |
| 2266 | UGCAUGCG G CGGUGAGC | 3995 | GCTCACCG GGCTAGCTACAACGA CGCATGCA | 8792 |
| 2263 | AUGCGGCG G UGAGCCUG | 3996 | CAGGCTCA GGCTAGCTACAACGA CGCCGCAT | 8793 |
| 2259 | GGCGGUGA G CCUGUGCU | 3997 | AGCACAGG GGCTAGCTACAACGA TCACCGCC | 8794 |
| 2255 | GUGAGCCU G UGCUCCAC | 3998 | GTGGAGCA GGCTAGCTACAACGA AGGCTCAC | 8795 |
| 2253 | GAGCCUGU G CUCCACGC | 3999 | GCGTGGAG GGCTAGCTACAACGA ACAGGCTC | 8796 |
| 2248 | UGUGCUCC A CGCCCCCC | 4000 | GGGGGGCG GGCTAGCTACAACGA GGAGCACA | 8797 |
| 2246 | UGCUCCAC G CCCCCCAC | 4001 | GTGGGGGG GGCTAGCTACAACGA GTGGAGCA | 8798 |
| 2239 | CGCCCCCC A CAUACAUC | 4002 | GATGTATG GGCTAGCTACAACGA GGGGGGCG | 8799 |
| 2237 | CCCCCCAC A UACAUCCU | 4003 | AGGATGTA GGCTAGCTACAACGA GTGGGGGG | 8800 |
| 2235 | CCCCACAU A CAUCCUAA | 4004 | TTAGGATG GGCTAGCTACAACGA ATGTGGGG | 8801 |
| 2233 | CCACAUAC A UCCUAACC | 4005 | GGTTAGGA GGCTAGCTACAACGA GTATGTGG | 8802 |
| 2227 | ACAUCCUA A CCUUAAAG | 4006 | CTTTAAGG GGCTAGCTACAACGA TAGGATGT | 8803 |
| 2218 | CCUUAAAG A UGGAAAAA | 4007 | TTTTTCCA GGCTAGCTACAACGA CTTTAAGG | 8804 |
| 2210 | AUGGAAAA A UUGACAGU | 4008 | ACTGTCAA GGCTAGCTACAACGA TTTTCCAT | 8805 |
| 2206 | AAAAAUUG A CAGUGCAG | 4009 | CTGCACTG GGCTAGCTACAACGA CAATTTTT | 8806 |
| 2203 | AAUUGACA G UGCAGGGG | 4010 | CCCCTGCA GGCTAGCTACAACGA TGTCAATT | 8807 |
| 2201 | UUGACAGU G CAGGGGUA | 4011 | TACCCCTG GGCTAGCTACAACGA ACTGTCAA | 8808 |
| 2195 | GUGCAGGG G UAGUGCCA | 4012 | TGGCACTA GGCTAGCTACAACGA CCCTGCAC | 8809 |
| 2292 | CAGGGGUA G UGCCAAAG | 4013 | CTTTGGCA GGCTAGCTACAACGA TACCCCTG | 8810 |
| 2190 | GGGGUAGU G CCAAAGCC | 4014 | GGCTTTGG GGCTAGCTACAACGA ACTACCCC | 8811 |
| 2184 | GUGCCAAA G CCUGUAUG | 4015 | CATACAGG GGCTAGCTACAACGA TTTGGCAC | 8812 |
| 2180 | CAAAGCCU G UAUGGGUA | 4016 | TACCCATA GGCTAGCTACAACGA AGGCTTTG | 8813 |
| 2178 | AAGCCUGU A UGGGUAGU | 4017 | ACTACCCA GGCTAGCTACAACGA ACAGGCTT | 8814 |
| 2174 | CUGUAUGG G UAGUCAAC | 4018 | GTTGACTA GGCTAGCTACAACGA CCATACAG | 8815 |
| 2171 | UAUGGGUA G UCAACUAU | 4019 | ATAGTTGA GGCTAGCTACAACGA TACCCATA | 8816 |
| 2167 | GGUAGUCA A CUAUGCAU | 4020 | ATGCATAG GGCTAGCTACAACGA TGACTACC | 8817 |
| 2164 | AGUCAACU A UGCAUCUA | 4021 | TAGATGCA GGCTAGCTACAACGA AGTTGACT | 8818 |
| 2162 | UCAACUAU G CAUCUAGG | 4022 | CCTAGATG GGCTAGCTACAACGA ATAGTTGA | 8819 |
| 2160 | AACUAUGC A UCUAGGUG | 4023 | CACCTAGA GGCTAGCTACAACGA GCATAGTT | 8820 |
| 2154 | GCAUCUAG G UGUUAACC | 4024 | GGTTAACA GGCTAGCTACAACGA CTAGATGC | 8821 |
| 2152 | AUCUAGGU G UUAACCAA | 4025 | TTGGTTAA GGCTAGCTACAACGA ACCTAGAT | 8822 |
| 2148 | AGGUGUUA A CCAAGGCC | 4026 | GGCCTTGG GGCTAGCTACAACGA TAACACCT | 8823 |
| 2142 | UAACCAAG G CCCCGAAC | 4027 | GTTCGGGG GGCTAGCTACAACGA CTTGGTTA | 8824 |
| 2135 | GGCCCCGA A CCGCACUU | 4028 | AAGTGCGG GGCTAGCTACAACGA TCGGGGCC | 8825 |
| 2132 | CCCGAACC G CACUUUGC | 4029 | GCAAAGTG GGCTAGCTACAACGA GGTTCGGG | 8826 |
| 2130 | CGAACCGC A CUUUGCGU | 4030 | ACGCAAAG GGCTAGCTACAACGA GCGGTTCG | 8827 |
| 2125 | CGCACUUU G CGUAAGUG | 4031 | CACTTACG GGCTAGCTACAACGA AAAGTGCG | 8828 |
| 2123 | CACUUUGC G UAAGUGGC | 4032 | GCCACTTA GGCTAGCTACAACGA GCAAAGTG | 8829 |
| 2119 | UUGCGUAA G UGGCCUCG | 4033 | CGAGGCCA GGCTAGCTACAACGA TTACGCAA | 8830 |
| 2116 | CGUAAGUG G CCUCGGGG | 4034 | CCCCGAGG GGCTAGCTACAACGA CACTTACG | 8831 |
| 2108 | GCCUCGGG G UGCUUCCG | 4035 | CGGAAGCA GGCTAGCTACAACGA CCCGAGGC | 8832 |
| 2106 | CUCGGGGU G CUUCCGGA | 4036 | TCCGGAAG GGCTAGCTACAACGA ACCCCGAG | 8833 |
| 2096 | UUCCGGAA G CAGUCCGU | 4037 | ACGGACTG GGCTAGCTACAACGA TTCCGGAA | 8834 |
| 2093 | CGGAAGCA G UCCGUGGG | 4038 | CCCACGGA GGCTAGCTACAACGA TGCTTCCG | 8835 |
| 2089 | AGCAGUCC G UGGGGCAG | 4039 | CTGCCCCA GGCTAGCTACAACGA GGACTGCT | 8836 |
| 2084 | UCCGUGGG G CAGGUUAA | 4040 | TTAACCTG GGCTAGCTACAACGA CCCACGGA | 8837 |
| 2080 | UGGGGCAG G UUAAGGUG | 4041 | CACCTTAA GGCTAGCTACAACGA CTGCCCCA | 8838 |
| 2074 | AGGUUAAG G UGUCGUUA | 4042 | TAACGACA GGCTAGCTACAACGA CTTAACCT | 8839 |
| 2072 | GUUAAGGU G UCGUUACC | 4043 | GGTAACGA GGCTAGCTACAACGA ACCTTAAC | 8840 |
| 2069 | AAGGUGUC G UUACCGGC | 4044 | GCCGGTAA GGCTAGCTACAACGA GACACCTT | 8841 |
| 2066 | GUGUCGUU A CCGGCCCC | 4045 | GGGGCCGG GGCTAGCTACAACGA AACGACAC | 8842 |
| 2062 | CGUUACCG G CCCCCCCG | 4046 | CGGGGGGG GGCTAGCTACAACGA CGGTAACG | 8843 |
| 2053 | CCCCCCCG A UGUUGCAC | 4047 | GTGCAACA GGCTAGCTACAACGA CGGGGGGG | 8844 |
| 2051 | CCCCCGAU G UUGCACGG | 4048 | CCGTGCAA GGCTAGCTACAACGA ATCGGGGG | 8845 |
| 2048 | CCGAUGUU G CACGGGGG | 4049 | CCCCCGTG GGCTAGCTACAACGA AACATCGG | 8846 |
| 2046 | GAUGUUGC A CGGGGGGC | 4050 | GCCCCCCG GGCTAGCTACAACGA GCAACATC | 8847 |
| 2039 | CACGGGGG G CCCCCGCA | 4051 | TGCGGGGG GGCTAGCTACAACGA CCCCCGTG | 8848 |
| 2033 | GGGCCCCC G CACGUCUU | 4052 | AAGACGTG GGCTAGCTACAACGA GGGGGCCC | 8849 |
| 2031 | GCCCCCGC A CGUCUUGG | 4053 | CCAAGACG GGCTAGCTACAACGA GCGGGGGC | 8850 |
| 2029 | CCCCGCAC G UCUUGGUG | 4054 | CACCAAGA GGCTAGCTACAACGA GTGCGGGG | 8851 |
| 2023 | ACGUCUUG G UGAACCCA | 4055 | TGGGTTCA GGCTAGCTACAACGA CAAGACGT | 8852 |
| 2019 | CUUGGUGA A CCCAGUGC | 4056 | GCACTGGG GGCTAGCTACAACGA TCACCAAG | 8853 |
| 2014 | UGAACCCA G UGCCAUUC | 4057 | GAATGGCA GGCTAGCTACAACGA TGGGTTCA | 8854 |
| 2012 | AACCCAGU G CCAUUCAU | 4058 | ATGAATGG GGCTAGCTACAACGA ACTGGGTT | 8855 |
| 2009 | CCAGUGCC A UUCAUCCA | 4059 | TGGATGAA GGCTAGCTACAACGA GGCACTGG | 8856 |
| 2005 | UGCCAUUC A UCCAUGUG | 4060 | CACATGGA GGCTAGCTACAACGA GAATGGCA | 8857 |
| 2001 | AUUCAUCC A UGUGCAGC | 4061 | GCTGCACA GGCTAGCTACAACGA GGATGAAT | 8858 |
| 1999 | UCAUCCAU G UGCAGCCG | 4062 | CGGCTGCA GGCTAGCTACAACGA ATGGATGA | 8859 |
| 1997 | AUCCAUGU G CAGCCGAA | 4063 | TTCGGCTG GGCTAGCTACAACGA ACATGGAT | 8860 |
| 1994 | CAUGUGCA G CCGAACCA | 4064 | TGGTTCGG GGCTAGCTACAACGA TGCACATG | 8861 |
| 1989 | GCAGCCGA A CCAGUUGC | 4065 | GCAACTGG GGCTAGCTACAACGA TCGGCTGC | 8862 |
| 1985 | CCGAACCA G UUGCCUUG | 4066 | CAAGGCAA GGCTAGCTACAACGA TGGTTCGG | 8863 |
| 1982 | AACCAGUU G CCUUGCGG | 4067 | CCGCAAGG GGCTAGCTACAACGA AACTGGTT | 8864 |
| 1977 | GUUGCCUU G CGGCGGCC | 4068 | GGCCGCCG GGCTAGCTACAACGA AAGGCAAC | 8865 |
| 1974 | GCCUUGCG G CGGCCGCG | 4069 | CGCGGCCG GGCTAGCTACAACGA CGCAAGGC | 8866 |
| 1971 | UUGCGGCG G CCGCGUGU | 4070 | ACACGCGG GGCTAGCTACAACGA CGCCGCAA | 8867 |
| 1968 | CGGCGGCC G CGUGUUGU | 4071 | ACAACACG GGCTAGCTACAACGA GGCCGCCG | 8868 |
| 1966 | GCGGCCGC G UGUUGUUG | 4072 | CAACAACA GGCTAGCTACAACGA GCGGCCGC | 8869 |
| 1964 | GGCCGCGU G UUGUUGAG | 4073 | CTCAACAA GGCTAGCTACAACGA ACGCGGCC | 8870 |
| 1961 | CGCGUGUU G UUGAGGAG | 4074 | CTCCTCAA GGCTAGCTACAACGA AACACGCG | 8871 |
| 1953 | GUUGAGGA G CAGCACGU | 4075 | ACGTGCTG GGCTAGCTACAACGA TCCTCAAC | 8872 |
| 1950 | GAGGAGCA G CACGUCCG | 4076 | CGGACGTG GGCTAGCTACAACGA TGCTCCTC | 8873 |
| 1948 | GGAGCAGC A CGUCCGUC | 4077 | GACGGACG GGCTAGCTACAACGA GCTGCTCC | 8874 |
| 1946 | AGCAGCAC G UCCGUCUC | 4078 | GAGACGGA GGCTAGCTACAACGA GTGCTGCT | 8875 |
| 1942 | GCACGUCC G UCUCGUUC | 4079 | GAACGAGA GGCTAGCTACAACGA GGACGTGC | 8876 |
| 1937 | UCCGUCUC G UUCGCCCC | 4080 | GGGGCGAA GGCTAGCTACAACGA GAGACGGA | 8877 |
| 1933 | UCUCGUUC G CCCCCCAG | 4081 | CTGGGGGG GGCTAGCTACAACGA GAACGAGA | 8878 |
| 1925 | GCCCCCCA G UUAUACGU | 4082 | ACGTATAA GGCTAGCTACAACGA TGGGGGGC | 8879 |
| 1922 | CCCCAGUU A UACGUGGG | 4083 | CCCACGTA GGCTAGCTACAACGA AACTGGGG | 8880 |
| 1920 | CCAGUUAU A CGUGGGGG | 4084 | CCCCCACG GGCTAGCTACAACGA ATAACTGG | 8881 |
| 1918 | AGUUAUAC G UGGGGGCG | 4085 | CGCCCCCA GGCTAGCTACAACGA GTATAACT | 8882 |
| 1912 | ACGUGGGG G CGCCGAAA | 4086 | TTTCGGCG GGCTAGCTACAACGA CCCCACGT | 8883 |
| 1910 | GUGGGGGC G CCGAAACG | 4087 | CGTTTCGG GGCTAGCTACAACGA GCCCCCAC | 8884 |
| 1904 | GCGCCGAA A CGGUCGGU | 4088 | ACCGACCG GGCTAGCTACAACGA TTCGGCGC | 8885 |
| 1901 | CCGAAACG G UCGGUCGU | 4089 | ACGACCGA GGCTAGCTACAACGA CGTTTCGG | 8886 |
| 1897 | AACGGUCG G UCGUCCCC | 4090 | GGGGACGA GGCTAGCTACAACGA CGACCGTT | 8887 |
| 1894 | GGUCGGUC G UCCCCACC | 4091 | GGTGGGGA GGCTAGCTACAACGA CACCGACC | 8888 |
| 1888 | UCGUCCCC A CCACAACA | 4092 | TGTTGTGG GGCTAGCTACAACGA GGGGACGA | 8889 |
| 1885 | UCCCCACC A CAACAGGG | 4093 | CCCTGTTG GGCTAGCTACAACGA GGTGGGGA | 8890 |
| 1882 | CCACCACA A CAGGGCUU | 4094 | AAGCCCTG GGCTAGCTACAACGA TGTGGTGG | 8891 |
| 1877 | ACAACAGG G CUUGGGGU | 4095 | ACCCCAAG GGCTAGCTACAACGA CCTGTTGT | 8892 |
| 1870 | GGCUUGGG G UGAAGCAA | 4096 | TTGCTTCA GGCTAGCTACAACGA CCCAAGCC | 8893 |
| 1865 | GGGGUGAA G CAAUACAC | 4097 | GTGTATTG GGCTAGCTACAACGA TTCACCCC | 8894 |
| 1862 | GUGAAGCA A UACACUGG | 4098 | CCAGTGTA GGCTAGCTACAACGA TGCTTCAC | 8895 |
| 1860 | GAAGCAAU A CACUGGAC | 4099 | GTCCAGTG GGCTAGCTACAACGA ATTGCTTC | 8896 |
| 1858 | AGCAAUAC A CUGGACCA | 4100 | TGGTCCAG GGCTAGCTACAACGA GTATTGCT | 8897 |
| 1853 | UACACUGG A CCACAUAC | 4101 | GTATGTGG GGCTAGCTACAACGA CCAGTGTA | 8898 |
| 1850 | ACUGGACC A CAUACCUG | 4102 | CAGGTATG GGCTAGCTACAACGA GGTCCAGT | 8899 |
| 1848 | UGGACCAC A UACCUGCG | 4103 | CGCAGGTA GGCTAGCTACAACGA GTGGTCCA | 8900 |
| 1846 | GACCACAU A CCUGCGAU | 4104 | ATCGCAGG GGCTAGCTACAACGA ATGTGGTC | 8901 |
| 1842 | ACAUACCU G CGAUGCGG | 4105 | CCGCATCG GGCTAGCTACAACGA AGGTATGT | 8902 |
| 1839 | UACCUGCG A UGCGGGUA | 4106 | TACCCGCA GGCTAGCTACAACGA CGCAGGTA | 8903 |
| 1837 | CCUGCGAU G CGGGUACG | 4107 | CGTACCCG GGCTAGCTACAACGA ATCGCAGG | 8904 |
| 1833 | CGAUGCGG G UACGAUAC | 4108 | GTATCGTA GGCTAGCTACAACGA CCGCATCG | 8905 |
| 1831 | AUGCGGGU A CGAUACCA | 4109 | TGGTATCG GGCTAGCTACAACGA ACCCGCAT | 8906 |
| 1828 | CGGGUACG A UACCACAC | 4110 | GTGTGGTA GGCTAGCTACAACGA CGTACCCG | 8907 |
| 1826 | GGUACGAU A CCACACGG | 4111 | CCGTGTGG GGCTAGCTACAACGA ATCGTACC | 8908 |
| 1823 | ACGAUACC A CACGGCCG | 4112 | CGGCCGTG GGCTAGCTACAACGA GGTATCGT | 8909 |
| 1821 | GAUACCAC A CGGCCGCG | 4113 | CGCGGCCG GGCTAGCTACAACGA GTGGTATC | 8910 |
| 1818 | ACCACACG G CCGCGGUG | 4114 | CACCGCGG GGCTAGCTACAACGA CGTGTGGT | 8911 |
| 1815 | ACACGGCC G CGGUGCGU | 4115 | ACGCACCG GGCTAGCTACAACGA GGCCGTGT | 8912 |
| 1812 | CGGCCGCG G UGCGUAGU | 4116 | ACTACGCA GGCTAGCTACAACGA CGCGGCCG | 8913 |
| 1810 | GCCGCGGU G CGUAGUGC | 4117 | GCACTACG GGCTAGCTACAACGA ACCGCGGC | 8914 |
| 1808 | CGCGGUGC G UAGUGCCA | 4118 | TGGCACTA GGCTAGCTACAACGA GCACCGCG | 8915 |
| 1805 | GGUGCGUA G UGCCAGCA | 4119 | TGCTGGCA GGCTAGCTACAACGA TACGCACC | 8916 |
| 1803 | UGCGUAGU G CCAGCAAU | 4120 | ATTGCTGG GGCTAGCTACAACGA ACTACGCA | 8917 |
| 1799 | UAGUGCCA G CAAUAGGG | 4121 | CCCTATTG GGCTAGCTACAACGA TGGCACTA | 8918 |
| 1796 | UGCCAGCA A UAGGGCCU | 4122 | AGGCCCTA GGCTAGCTACAACGA TGCTGGCA | 8919 |
| 1791 | GCAAUAGG G CCUCUGGU | 4123 | ACCAGAGG GGCTAGCTACAACGA CCTATTGC | 8920 |
| 1784 | GGCCUCUG G UCCGAGUU | 4124 | AACTCGGA GGCTAGCTACAACGA CAGAGGCC | 8921 |
| 1778 | UGGUCCGA G UUGUGGCC | 4125 | GGCCACAA GGCTAGCTACAACGA TCGGACCA | 8922 |
| 1775 | UCCGAGUU G UGGCCCUC | 4126 | GAGGGCCA GGCTAGCTACAACGA AACTCGGA | 8923 |
| 1772 | GAGUUGUG G CCCUCGGU | 4127 | ACCGAGGG GGCTAGCTACAACGA CACAACTC | 8924 |
| 1765 | GGCCCUCG G UGUAGGUG | 4128 | CACCTACA GGCTAGCTACAACGA CGAGGGCC | 8925 |
| 1763 | CCCUCGGU G UAGGUGAU | 4129 | ATCACCTA GGCTAGCTACAACGA ACCGAGGG | 8926 |
| 1759 | CGGUGUAG G UGAUAGGA | 4130 | TCCTATCA GGCTAGCTACAACGA CTACACCG | 8927 |
| 1756 | UGUAGGUG A UAGGACCC | 4131 | GGGTCCTA GGCTAGCTACAACGA CACCTACA | 8928 |
| 1751 | GUGAUAGG A CCCCACCC | 4132 | GGGTGGGG GGCTAGCTACAACGA CCTATCAC | 8929 |
| 1746 | AGGACCCC A CCCCUGAG | 4133 | CTCAGGGG GGCTAGCTACAACGA GGGGTCCT | 8930 |
| 1738 | ACCCCUGA G CGAACUUG | 4134 | CAAGTTCG GGCTAGCTACAACGA TCAGGGGT | 8931 |
| 1734 | CUGAGCGA A CUUGUCAA | 4135 | TTGACAAG GGCTAGCTACAACGA TCGCTCAG | 8932 |
| 1730 | GCGAACUU G UCAAUGGA | 4136 | TCCATTGA GGCTAGCTACAACGA AAGTTCGC | 8933 |
| 1726 | ACUUGUCA A UGGAGCGG | 4137 | CCGCTCCA GGCTAGCTACAACGA TGACAAGT | 8934 |
| 1721 | UCAAUGGA G CGGCAGCU | 4138 | AGCTGCCG GGCTAGCTACAACGA TCCATTGA | 8935 |
| 1718 | AUGGAGCG G CAGCUGGC | 4139 | GCCAGCTG GGCTAGCTACAACGA CGCTCCAT | 8936 |
| 1715 | GAGCGGCA G CUGGCCAA | 4140 | TTGGCCAG GGCTAGCTACAACGA TGCCGCTC | 8937 |
| 1711 | GGCAGCUG G CCAAGCGC | 4141 | GCGCTTGG GGCTAGCTACAACGA CAGCTGCC | 8938 |
| 1706 | CUGGCCAA G CGCUGUGG | 4142 | CCACAGCG GGCTAGCTACAACGA TTGGCCAG | 8939 |
| 1704 | GGCCAAGC G CUGUGGGC | 4143 | GCCCACAG GGCTAGCTACAACGA GCTTGGCC | 8940 |
| 1701 | CAAGCGCU G UGGGCAUC | 4144 | GATGCCCA GGCTAGCTACAACGA AGCGCTTG | 8941 |
| 1697 | CGCUGUGG G CAUCCGGA | 4145 | TCCGGATG GGCTAGCTACAACGA CCACAGCG | 8942 |
| 1695 | CUGUGGGC A UCCGGACG | 4146 | CGTCCGGA GGCTAGCTACAACGA GCCCACAG | 8943 |
| 1689 | GCAUCCGG A CGAGUUGA | 4147 | TCAACTCG GGCTAGCTACAACGA CCGGATGC | 8944 |
| 1685 | CCGGACGA G UUGAACCU | 4148 | AGGTTCAA GGCTAGCTACAACGA TCGTCCGG | 8945 |
| 1680 | CGAGUUGA A CCUGUGUG | 4149 | CACACAGG GGCTAGCTACAACGA TCAACTCG | 8946 |
| 1676 | UUGAACCU G UGUGCAUA | 4150 | TATGCACA GGCTAGCTACAACGA AGGTTCAA | 8947 |
| 1674 | GAACCUGU G UGCAUAGA | 4151 | TCTATGCA GGCTAGCTACAACGA ACAGGTTC | 8948 |
| 1672 | ACCUGUGU G CAUAGAAC | 4152 | GTTCTATG GGCTAGCTACAACGA ACACAGGT | 8949 |
| 1670 | CUGUGUGC A UAGAACAG | 4153 | CTGTTCTA GGCTAGCTACAACGA GCACACAG | 8950 |
| 1665 | UGCAUAGA A CAGUGCAG | 4154 | CTGCACTG GGCTAGCTACAACGA TCTATGCA | 8951 |
| 1662 | AUAGAACA G UGCAGCAA | 4155 | TTGCTGCA GGCTAGCTACAACGA TGTTCTAT | 8952 |
| 1660 | AGAACAGU G CAGCAAUG | 4156 | CATTGCTG GGCTAGCTACAACGA ACTGTTCT | 8953 |
| 1657 | ACAGUGCA G CAAUGAAC | 4157 | GTTCATTG GGCTAGCTACAACGA TGCACTGT | 8954 |
| 1654 | GUGCAGCA A UGAACCCG | 4158 | CGGGTTCA GGCTAGCTACAACGA TGCTGCAC | 8955 |
| 1650 | AGCAAUGA A CCCGGUUU | 4159 | AAACCGGG GGCTAGCTACAACGA TCATTGCT | 8956 |
| 1645 | UGAACCCG G UUUGGAGG | 4160 | CCTCCAAA GGCTAGCTACAACGA CGGGTTCA | 8957 |
| 1634 | UGGAGGGA G UCAUUGCA | 4161 | TGCAATGA GGCTAGCTACAACGA TCCCTCCA | 8958 |
| 1631 | AGGGAGUC A UUGCAGUU | 4162 | AACTGCAA GGCTAGCTACAACGA GACTCCCT | 8959 |
| 1628 | GAGUCAUU G CAGUUCAG | 4163 | CTGAACTG GGCTAGCTACAACGA AATGACTC | 8960 |
| 1625 | UCAUUGCA G UUCAGGGC | 4164 | GCCCTGAA GGCTAGCTACAACGA TGCAATGA | 8961 |
| 1618 | AGUUCAGG G CAGUCCUG | 4165 | CAGGACTG GGCTAGCTACAACGA CCTGAACT | 8962 |
| 1615 | UCAGGGCA G UCCUGUUA | 4166 | TAACAGGA GGCTAGCTACAACGA TGCCCTGA | 8963 |
| 1610 | GCAGUCCU G UUAAUGUG | 4167 | CACATTAA GGCTAGCTACAACGA AGGACTGC | 8964 |
| 1606 | UCCUGUUA A UGUGCCAG | 4168 | CTGGCACA GGCTAGCTACAACGA TAACAGGA | 8965 |
| 1604 | CUGUUAAU G UGCCAGCU | 4169 | AGCTGGCA GGCTAGCTACAACGA ATTAACAG | 8966 |
| 1602 | GUUAAUGU G CCAGCUGC | 4170 | GCAGCTGG GGCTAGCTACAACGA ACATTAAC | 8967 |
| 1598 | AUGUGCCA G CUGCCGUU | 4171 | AACGGCAG GGCTAGCTACAACGA TGGCACAT | 8968 |
| 1595 | UGCCAGCU G CCGUUGGC | 4172 | ACCAACGG GGCTAGCTACAACGA AGCTGGCA | 8969 |
| 1592 | CAGCUGCC G UUGGUGUU | 4173 | AACACCAA GGCTAGCTACAACGA GGCAGCTG | 8970 |
| 1588 | UGCCGUUG G UGUUAAUA | 4174 | TATTAACA GGCTAGCTACAACGA CAACGGCA | 8971 |
| 1586 | CCGUUGGU G UUAAUAAG | 4175 | CTTATTAA GGCTAGCTACAACGA ACCAACGG | 8972 |
| 1582 | UGGUGUUA A UAAGCUGG | 4176 | CCAGCTTA GGCTAGCTACAACGA TAACACCA | 8973 |
| 1578 | GUUAAUAA G CUGGAUAU | 4177 | ATATCCAG GGCTAGCTACAACGA TTATTAAC | 8974 |
| 1573 | UAAGCUGG A UAUUCUGA | 4178 | TCAGAATA GGCTAGCTACAACGA CCAGCTTA | 8975 |
| 1571 | AGCUGGAU A UUCUGAGA | 4179 | TCTCAGAA GGCTAGCTACAACGA ATCCAGCT | 8976 |
| 1563 | AUUCUGAG A UGCUCCAG | 4180 | CTGGAGCA GGCTAGCTACAACGA CTCAGAAT | 8977 |
| 1561 | UCUGAGAU G CUCCAGAU | 4181 | ATCTGGAG GGCTAGCTACAACGA ATCTCAGA | 8978 |
| 1554 | UGCUCCAG A UGUAAAGA | 4182 | TCTTTACA GGCTAGCTACAACGA CTGGAGCA | 8979 |
| 1552 | CUCCAGAU G UAAAGAGG | 4183 | CCTCTTTA GGCTAGCTACAACGA ATCTGGAG | 8980 |
| 1542 | AAAGAGGG A UGCCACCC | 4184 | GGGTGGCA GGCTAGCTACAACGA CCCTCTTT | 8981 |
| 1540 | AGAGGGAU G CCACCCUA | 4185 | TAGGGTGG GGCTAGCTACAACGA ATCCCTCT | 8982 |
| 1537 | GGGAUGCC A CCCUACUA | 4186 | TAGTAGGG GGCTAGCTACAACGA GGCATCCC | 8983 |
| 1532 | GCCACCCU A CUAGUGGU | 4187 | ACCACTAG GGCTAGCTACAACGA AGGGTGGC | 8984 |
| 1528 | CCCUACUA G UGGUGUGG | 4188 | CCACACCA GGCTAGCTACAACGA TAGTAGGG | 8985 |
| 1525 | UACUAGUG G UGUGGCCC | 4189 | GGGCCACA GGCTAGCTACAACGA CACTAGTA | 8986 |
| 1523 | CUAGUGGU G UGGCCCUG | 4190 | CAGGGCCA GGCTAGCTACAACGA ACCACTAG | 8987 |
| 1520 | GUGGUGUG G CCCUGCGC | 4191 | GCGCAGGG GGCTAGCTACAACGA CACACCAC | 8988 |
| 1515 | GUGGCCCU G CGCCCCCC | 4192 | GGGGGGCG GGCTAGCTACAACGA AGGGCCAC | 8989 |
| 1513 | GGCCCUGC G CCCCCCCU | 4193 | AGGGGGGG GGCTAGCTACAACGA GCAGGGCC | 8990 |
| 1504 | CCCCCCCU G UCGUGUAG | 4194 | CTACACGA GGCTAGCTACAACGA AGGGGGGG | 8991 |
| 1501 | CCCCUGUC G UGUAGGUG | 4195 | CACCTACA GGCTAGCTACAACGA GACAGGGG | 8992 |
| 1499 | CCUGUCGU G UAGGUGUC | 4196 | GACACCTA GGCTAGCTACAACGA ACGACAGG | 8993 |
| 1495 | UCGUGUAG G UGUCCCCG | 4197 | CGGGGACA GGCTAGCTACAACGA CTACACGA | 8994 |
| 1493 | GUGUAGGU G UCCCCGUC | 4198 | GACGGGGA GGCTAGCTACAACGA ACCTACAC | 8995 |
| 1487 | GUGUCCCC G UCAACGCC | 4199 | GGCGTTGA GGCTAGCTACAACGA GGGGACAC | 8996 |
| 1483 | CCCCGUCA A CGCCGGCA | 4200 | TGCCGGCG GGCTAGCTACAACGA TGACGGGG | 8997 |
| 1481 | CCGUCAAC G CCGGCAAA | 4201 | TTTGCCGG GGCTAGCTACAACGA GTTGACGG | 8998 |
| 1477 | CAACGCCG G CAAAGAGU | 4202 | ACTCTTTG GGCTAGCTACAACGA CGGCGTTG | 8999 |
| 1470 | GGCAAAGA G UAGCAUCA | 4203 | TGATGCTA GGCTAGCTACAACGA TCTTTGCC | 9000 |
| 1467 | AAAGAGUA G CAUCACAA | 4204 | TTGTGATG GGCTAGCTACAACGA TACTCTTT | 9001 |
| 1465 | AGAGUAGC A UCACAAUC | 4205 | GATTGTGA GGCTAGCTACAACGA GCTACTCT | 9002 |
| 1462 | GUAGCAUC A CAAUCAAC | 4206 | GTTGATTG GGCTAGCTACAACGA GATGCTAC | 9003 |
| 1459 | GCAUCACA A UCAACACC | 4207 | GGTGTTGA GGCTAGCTACAACGA TGTGATGC | 9004 |
| 1455 | CACAAUCA A CACCUUAG | 4208 | CTAAGGTG GGCTAGCTACAACGA TGATTGTG | 9005 |
| 1453 | CAAUCAAC A CCUUAGCC | 4209 | GGCTAAGG GGCTAGCTACAACGA GTTGATTG | 9006 |
| 1447 | ACACCUUA G CCCAGUUC | 4210 | GAACTGGG GGCTAGCTACAACGA TAAGGTGT | 9007 |
| 1442 | UUAGCCCA G UUCCCCAC | 4211 | TTCCATGG GGCTAGCTACAACGA GGGGAACT | 9008 |
| 1435 | AGUUCCCC A CCAUGGAA | 4212 | TTCCATGG GGCTAGCTACAACGA GGGGAACT | 9009 |
| 1432 | UCCCCACC A UGGAAUAA | 4213 | TTATTCCA GGCTAGCTACAACGA GGTGGGGA | 9010 |
| 1427 | ACCAUGGA A UAAUAGGC | 4214 | GCCTATTA GGCTAGCTACAACGA TCCATGGT | 9011 |
| 1424 | AUGGAAUA A UAGGCAAG | 4215 | CTTGCCTA GGCTAGCTACAACGA TATTCCAT | 9012 |
| 1420 | AAUAAUAG G CAAGGCCC | 4216 | GGGCCTTG GGCTAGCTACAACGA CTATTATT | 9013 |
| 1415 | UAGGCAAG G CCCGCCAG | 4217 | CTGGCGGG GGCTAGCTACAACGA CTTGCCTA | 9014 |
| 1411 | CAAGGCCC G CCAGGACU | 4218 | AGTCCTGG GGCTAGCTACAACGA GGGCCTTG | 9015 |
| 1405 | CCGCCAGG A CUCCCCAG | 4219 | CTGGGGAG GGCTAGCTACAACGA CCTGGCGG | 9016 |
| 1397 | ACUCCCCA G UGGGCCCC | 4220 | GGGGCCCA GGCTAGCTACAACGA TGGGGAGT | 9017 |
| 1393 | CCCAGUGG G CCCCCGCC | 4221 | GGCGGGGG GGCTAGCTACAACGA CCACTGGG | 9018 |
| 1387 | GGGCCCCC G CCACCAUG | 4222 | CATGGTGG GGCTAGCTACAACGA GGGGGCCC | 9019 |
| 1384 | CCCCCGCC A CCAUGUCC | 4223 | GGACATGG GGCTAGCTACAACGA GGCGGGGG | 9020 |
| 1381 | CCGCCACC A UGUCCACG | 4224 | CGTGGACA GGCTAGCTACAACGA GGTGGCGG | 9021 |
| 1379 | GCCACCAU G UCCACGAC | 4225 | GTCGTGGA GGCTAGCTACAACGA ATGGTGGC | 9022 |
| 1375 | CCAUGUCC A CGACGGCU | 4226 | AGCCGTCG GGCTAGCTACAACGA GGACATGG | 9023 |
| 1372 | UGUCCACG A CGGCUUGU | 4227 | ACAAGCCG GGCTAGCTACAACGA CGTGGACA | 9024 |
| 1369 | CCACGACG G CUUGUGGG | 4228 | CCCACAAG GGCTAGCTACAACGA CGTCGTGG | 9025 |
| 1365 | GACGGCUU G UGGGAUCC | 4229 | GGATCCCA GGCTAGCTACAACGA AAGCCGTC | 9026 |
| 1360 | CUUGUGGG A UCCGGAGC | 4230 | GCTCCGGA GGCTAGCTACAACGA CCCACAAG | 9027 |
| 1353 | GAUCCGGA G CAACUGCG | 4231 | CGCAGTTG GGCTAGCTACAACGA TCCGGATC | 9028 |
| 1350 | CCGGAGCA A CUGCGAUA | 4232 | TATCGCAG GGCTAGCTACAACGA TGCTCCGG | 9029 |
| 1347 | GAGCAACU G CGAUACCA | 4233 | TGGTATCG GGCTAGCTACAACGA AGTTGCTC | 9030 |
| 1344 | CAACUGCG A UACCACUA | 4234 | TAGTGGTA GGCTAGCTACAACGA CGCAGTTG | 9031 |
| 1342 | ACUGCGAU A CCACUAGG | 4235 | CCTAGTGG GGCTAGCTACAACGA ATCGCAGT | 9032 |
| 1339 | GCGAUACC A CUAGGGCU | 4236 | AGCCCTAG GGCTAGCTACAACGA GGTATCGC | 9033 |
| 1333 | CCACUAGG G CUGUUGUA | 4237 | TACAACAG GGCTAGCTACAACGA CCTAGTGG | 9034 |
| 1330 | CUAGGGCU G UUGUAGGU | 4238 | ACCTACAA GGCTAGCTACAACGA AGCCCTAG | 9035 |
| 1327 | GGGCUGUU G UAGGUGAC | 4239 | GTCACCTA GGCTAGCTACAACGA AACAGCCC | 9036 |
| 1323 | UGUUGUAG G UGACCAAU | 4240 | ATTGGTCA GGCTAGCTACAACGA CTACAACA | 9037 |
| 1320 | UGUAGGUG A CCAAUUCA | 4241 | TGAATTGG GGCTAGCTACAACGA CACCTACA | 9038 |
| 1316 | GGUGACCA A UUCAUCAU | 4242 | ATGATGAA GGCTAGCTACAACGA TGGTCACC | 9039 |
| 1312 | ACCAAUUC A UCAUCAUA | 4243 | TATGATGA GGCTAGCTACAACGA GAATTGGT | 9040 |
| 1309 | AAUUCAUC A UCAUAUCC | 4244 | GGATATGA GGCTAGCTACAACGA GATGAATT | 9041 |
| 1306 | UCAUCAUC A UAUCCCAA | 4245 | TTGGGATA GGCTAGCTACAACGA GATGATGA | 9042 |
| 1304 | AUCAUCAU A UCCCAAGC | 4246 | GCTTGGGA GGCTAGCTACAACGA ATGATGAT | 9043 |
| 1297 | UAUCCCAA G CCAUGCGA | 4247 | TCGCATGG GGCTAGCTACAACGA TTGGGATA | 9044 |
| 1294 | CCCAAGCC A UGCGAUGG | 4248 | CCATCGCA GGCTAGCTACAACGA GGCTTGGG | 9045 |
| 1292 | CAAGCCAU G CGAUGGCC | 4249 | GGCCATCG GGCTAGCTACAACGA ATGGCTTG | 9046 |
| 1289 | GCCAUGCG A UGGCCUGA | 4250 | TCAGGCCA GGCTAGCTACAACGA CGCATGGC | 9047 |
| 1286 | AUGCGAUG G CCUGAUAC | 4251 | GTATCAGG GGCTAGCTACAACGA CATCGCAT | 9048 |
| 1281 | AUGGCCUG A UACGUGGC | 4252 | GCCACGTA GGCTAGCTACAACGA CAGGCCAT | 9049 |
| 1279 | GGCCUGAU A CGUGGCCG | 4253 | CGGCCACG GGCTAGCTACAACGA ATCAGGCC | 9050 |
| 1277 | CCUGAUAC G UGGCCGGG | 4254 | CCCGGCCA GGCTAGCTACAACGA GTATCAGG | 9051 |
| 1274 | GAUACGUG G CCGGGAUA | 4255 | TATCCCGG GGCTAGCTACAACGA CACGTATC | 9052 |
| 1268 | UGGCCGGG A UAGAUCGA | 4256 | TCGATCTA GGCTAGCTACAACGA CCCGGCCA | 9053 |
| 1264 | CGGGAUAG A UCGAGCAA | 4257 | TTGCTCGA GGCTAGCTACAACGA CTATCCCG | 9054 |
| 1259 | UAGAUCGA G CAAUUACA | 4258 | TGTAATTG GGCTAGCTACAACGA TCGATCTA | 9055 |
| 1256 | AUCGAGCA A UUACAGUC | 4259 | GACTGTAA GGCTAGCTACAACGA TGCTCGAT | 9056 |
| 1253 | GAGCAAUU A CAGUCCUG | 4260 | CAGGACTG GGCTAGCTACAACGA AATTGCTC | 9057 |
| 1250 | CAAUUACA G UCCUGUAC | 4261 | GTACAGGA GGCTAGCTACAACGA TGTAATTG | 9058 |
| 1245 | ACAGUCCU G UACUGUCU | 4262 | AGACAGTA GGCTAGCTACAACGA AGGACTGT | 9059 |
| 1243 | AGUCCUGU A CUGUCUCA | 4263 | TGAGACAG GGCTAGCTACAACGA ACAGGACT | 9060 |
| 1240 | CCUGUACU G UCUCAUAC | 4264 | GTATGAGA GGCTAGCTACAACGA AGTACAGG | 9061 |
| 1235 | ACUGUCUC A UACCGGCG | 4265 | CGCCGGTA GGCTAGCTACAACGA GAGACAGT | 9062 |
| 1233 | UGUCUCAU A CCGGCGAG | 4266 | CTCGCCGG GGCTAGCTACAACGA ATGAGACA | 9063 |
| 1229 | UCAUACCG G CGAGGCGA | 4267 | TCGCCTCG GGCTAGCTACAACGA CGGTATGA | 9064 |
| 1224 | CCGGCGAG G CGAGAAGG | 4268 | CCTTCTCG GGCTAGCTACAACGA CTCGCCGG | 9065 |
| 1216 | GCGAGAAG G UGAACAGC | 4269 | GCTGTTCA GGCTAGCTACAACGA CTTCTCGC | 9066 |
| 1212 | GAAGGUGA A CAGCUGAG | 4270 | CTCAGCTG GGCTAGCTACAACGA TCACCTTC | 9067 |
| 1209 | GGUGAACA G CUGAGAGA | 4271 | TCTCTCAG GGCTAGCTACAACGA TGTTCACC | 9068 |
| 1201 | GCUGAGAG A CGAGGAAG | 4272 | CTTCCTCG GGCTAGCTACAACGA CTCTCAGC | 9069 |
| 1192 | CGAGGAAG A CAGAUCCG | 4273 | CGGATCTG GGCTAGCTACAACGA CTTCCTCG | 9070 |
| 1188 | GAAGACAG A UCCGCAGA | 4274 | TCTGCGGA GGCTAGCTACAACGA CTGTCTTC | 9071 |
| 1184 | ACAGAUCC G CAGAGAUC | 4275 | GATCTCTG GGCTAGCTACAACGA GGATCTGT | 9072 |
| 1178 | CCGCAGAG A UCCCCCAC | 4276 | GTGGGGGA GGCTAGCTACAACGA CTCTGCCG | 9073 |
| 1171 | GAUCCCCC A CGUACAUA | 4277 | TATGTACG GGCTAGCTACAACGA GGGGGATC | 9074 |
| 1169 | UCCCCCAC G UACAUAGC | 4278 | GCTATGTA GGCTAGCTACAACGA GTGGGGGA | 9075 |
| 1167 | CCCCACGU A CAUAGCAG | 4279 | CTGCTATG GGCTAGCTACAACGA ACGTGGGG | 9076 |
| 1165 | CCACGUAC A UAGCAGAG | 4280 | CTCTGCTA GGCTAGCTACAACGA GTACGTGG | 9077 |
| 1162 | CGUACAUA G CAGAGCAG | 4281 | CTGCTCTG GGCTAGCTACAACGA TATGTACG | 9078 |
| 1157 | AUAGCAGA G CAGAAAGC | 4282 | GCTTTCTG GGCTAGCTACAACGA TCTGCTAT | 9079 |
| 1150 | AGCAGAAA G CAGCCGCC | 4283 | GGCGGCTG GGCTAGCTACAACGA TTTCTGCT | 9080 |
| 1147 | AGAAAGCA G CCGCCCCA | 4284 | TGGGGCGG GGCTAGCTACAACGA TGCTTTCT | 9081 |
| 1144 | AAGCAGCC G CCCCAACG | 4285 | CGTTGGGG GGCTAGCTACAACGA GGCTGCTT | 9082 |
| 1138 | CCGCCCCA A CGAGCAAA | 4286 | TTTGCTCG GGCTAGCTACAACGA TGGGGCGG | 9083 |
| 1134 | CCCAACGA G CAAAUCGA | 4287 | TCGATTTG GGCTAGCTACAACGA TCGTTGGG | 9084 |
| 1130 | ACGAGCAA A UCGACGUG | 4288 | CACGTCGA GGCTAGCTACAACGA TTGCTCGT | 9085 |
| 1126 | GCAAAUCG A CGUGACGC | 4289 | GCGTCACG GGCTAGCTACAACGA CGATTTGC | 9086 |
| 1124 | AAAUCGAC G UGACGCCG | 4290 | CGGCGTCA GGCTAGCTACAACGA GTCGATTT | 9087 |
| 1121 | UCGACGUG A CGCCGUAU | 4291 | ATACGGCG GGCTAGCTACAACGA CACGTCGA | 9088 |
| 1119 | GACGUGAC G CCGUAUCG | 4292 | CGATACGG GGCTAGCTACAACGA GTCACGTC | 9089 |
| 1116 | GUGACGCC G UAUCGUCG | 4293 | CGACGATA GGCTAGCTACAACGA GGCGTCAC | 9090 |
| 1114 | GACGCCGU A UCGUCGUA | 4294 | TACGACGA GGCTAGCTACAACGA ACGGCGTC | 9091 |
| 1111 | GCCGUAUC G UCGUAGUG | 4295 | CACTACGA GGCTAGCTACAACGA GATACGGC | 9092 |
| 1108 | GUAUCGUC G UAGUGGGG | 4296 | CCCCACTA GGCTAGCTACAACGA GACGATAC | 9093 |
| 1105 | UCGUCGUA G UGGGGAUG | 4297 | CATCCCCA GGCTAGCTACAACGA TACGACGA | 9094 |
| 1099 | UAGUGGGG A UGCUGGCA | 4298 | TGCCAGCA GGCTAGCTACAACGA CCCCACTA | 9095 |
| 1097 | GUGGGGAU G CUGGCAUU | 4299 | AATGCCAG GGCTAGCTACAACGA ATCCCCAC | 9096 |
| 1093 | GGAUGCUG G CAUUCCUG | 4300 | CAGGAATG GGCTAGCTACAACGA CAGCATCC | 9097 |
| 1091 | AUGCUGGC A UUCCUGGC | 4301 | GCCAGGAA GGCTAGCTACAACGA GCCAGCAT | 9098 |
| 1084 | CAUUCCUG G CCGCGAGC | 4302 | GCTCGCGG GGCTAGCTACAACGA CAGGAATG | 9099 |
| 1081 | UCCUGGCC G CGAGCGUG | 4303 | CACGCTCG GGCTAGCTACAACGA GGCCAGGA | 9100 |
| 1077 | GGCCGCGA G CGUGGGAG | 4304 | CTCCCACG GGCTAGCTACAACGA TCGCGGCC | 9101 |
| 1075 | CCGCGAGC G UGGGAGUG | 4305 | CACTCCCA GGCTAGCTACAACGA GCTCGCGG | 9102 |
| 1069 | GCGUGGGA G UGAGCGCU | 4306 | AGCGCTCA GGCTAGCTACAACGA TCCCACGC | 9103 |
| 1065 | GGGAGUGA G CGCUACCC | 4307 | GGGTAGCG GGCTAGCTACAACGA TCACTCCC | 9104 |
| 1063 | GAGUGAGC G CUACCCAG | 4308 | CTGGGTAG GGCTAGCTACAACGA GCTCACTC | 9105 |
| 1060 | UGAGCGCU A CCCAGCAG | 4309 | CTGCTGGG GGCTAGCTACAACGA AGCGCTCA | 9106 |
| 1055 | GCUACCCA G CAGCGGGA | 4310 | TCCCGCTG GGCTAGCTACAACGA TGGGTAGC | 9107 |
| 1052 | ACCCAGCA G CGGGAGGA | 4311 | TCCTCCCG GGCTAGCTACAACGA TGCTGGGT | 9108 |
| 1043 | CGGGAGGA G UUGUUCUC | 4312 | GAGAACAA GGCTAGCTACAACGA TCCTCCCG | 9109 |
| 1040 | GAGGAGUU G UUCUCCCG | 4313 | CGGGAGAA GGCTAGCTACAACGA AACTCCTC | 9110 |
| 1030 | UCUCCCGA A CGCAGGGC | 4314 | GCCCTGCG GGCTAGCTACAACGA TCGGGAGA | 9111 |
| 1028 | UCCCGAAC G CAGGGCAC | 4315 | GTGCCCTG GGCTAGCTACAACGA GTTCGGGA | 9112 |
| 1023 | AACGCAGG G CACGCACC | 4316 | GGTGCGTG GGCTAGCTACAACGA CCTGCGTT | 9113 |
| 1021 | CGCAGGGC A CGCACCCC | 4317 | GGGGTGCG GGCTAGCTACAACGA GCCCTGCG | 9114 |
| 1019 | CAGGGCAC G CACCCCGG | 4318 | CCGGGGTG GGCTAGCTACAACGA GTGCCCTG | 9115 |
| 1017 | GGGCACGC A CCCCGGGG | 4319 | CCCCGGGG GGCTAGCTACAACGA GCGTGCCC | 9116 |
| 1009 | ACCCCGGG G UGUGCAUG | 4320 | CATCGACA GGCTAGCTACAACGA CCCGGGGT | 9117 |
| 1007 | CCCGGGGU G UGCAUGAU | 4321 | ATCATGCA GGCTAGCTACAACGA ACCCCGGG | 9118 |
| 1005 | CGGGGUGU G CAUGAUCA | 4322 | TGATCATG GGCTAGCTACAACGA ACACCCCG | 9119 |
| 1003 | GGGUGUGC A UGAUCAUG | 4323 | CATGATCA GGCTAGCTACAACGA GCACACCC | 9120 |
| 1000 | UGUGCAUG A UCAUGUCC | 4324 | GGACATGA GGCTAGCTACAACGA CATGCACA | 9121 |
| 997 | GCAUGAUC A UGUCCUCU | 4325 | AGAGGACA GGCTAGCTACAACGA GATCATGC | 9122 |
| 995 | AUGAUCAU G UCCUCUGC | 4326 | GCAGAGGA GGCTAGCTACAACGA ATGATCAT | 9123 |
| 988 | UGUCCUCU G CCUCAUAC | 4327 | GTATGAGG GGCTAGCTACAACGA AGAGGACA | 9124 |
| 983 | UCUGCCUC A UACACAAU | 4328 | ATTGTGTA GGCTAGCTACAACGA GAGGCAGA | 9125 |
| 981 | UGCCUCAU A CACAAUGC | 4329 | GCATTGTG GGCTAGCTACAACGA ATGAGGCA | 9128 |
| 979 | CCUCAUAC A CAAUGCUU | 4330 | AAGCATTG GGCTAGCTACAACGA GTATGAGG | 9127 |
| 976 | CAUACACA A UGCUUGAG | 4331 | CTCAAGCA GGCTAGCTACAACGA TGTGTATG | 9128 |
| 974 | UACACAAU G CUUGAGUU | 4332 | AACTCAAG GGCTAGCTACAACGA ATTGTGTA | 9129 |
| 968 | AUGCUUGA G UUGGAGCA | 4333 | TGCTCCAA GGCTAGCTACAACGA TCAAGCAT | 9130 |
| 962 | GAGUUGGA G CAAUCGUU | 4334 | AACGATTG GGCTAGCTACAACGA TCCAACTC | 9131 |
| 959 | UUGGAGCA A UCGUUCGU | 4335 | ACGAACGA GGCTAGCTACAACGA TGCTCCAA | 9132 |
| 956 | GAGCAAUC G UUCGUGAC | 4336 | GTCACGAA GGCTAGCTACAACGA GATTGCTC | 9133 |
| 952 | AAUCGUUC G UGACAUGG | 4337 | CCATGTCA GGCTAGCTACAACGA GAACGATT | 9134 |
| 949 | CGUUCGUG A CAUGGUAC | 4338 | GTACCATG GGCTAGCTACAACGA CACGAACG | 9135 |
| 947 | UUCGUGAC A UGGUACAG | 4339 | CTGTACCA GGCTAGCTACAACGA GTCACGAA | 9136 |
| 944 | GUGACAUG G UACAGCCC | 4340 | GGGCTGTA GGCTAGCTACAACGA CATGTCAC | 9137 |
| 942 | GACAUGGU A CAGCCCGG | 4341 | CCGGGCTG GGCTAGCTACAACGA ACCATGTC | 9138 |
| 939 | AUGGUACA G CCCGGACG | 4342 | CGTCCGGG GGCTAGCTACAACGA TGTACCAT | 9139 |
| 933 | CAGCCCGG A CGCGUUGC | 4343 | GCAACGCG GGCTAGCTACAACGA CCGGGCTG | 9140 |
| 931 | GCCCGGAC G CGUUGCAC | 4344 | GTGCAACG GGCTAGCTACAACGA GTCCGGGC | 9141 |
| 929 | CCGGACGC G UUGCACAC | 4345 | GTGTGCAA GGCTAGCTACAACGA GCGTCCGG | 9142 |
| 926 | GACGCGUU G CACACCUC | 4346 | GAGGTGTG GGCTAGCTACAACGA AACGCGTC | 9143 |
| 924 | CGCGUUGC A CACCUCAU | 4347 | ATGAGGTG GGCTAGCTACAACGA GCAACGCG | 9144 |
| 922 | CGUUGCAC A CCUCAUAA | 4348 | TTATGAGG GGCTAGCTACAACGA GTGCAACG | 9145 |
| 917 | CACACCUC A UAAGCGGA | 4349 | TCCGCTTA GGCTAGCTACAACGA GAGGTGTG | 9146 |
| 913 | CCUCAUAA G CGGAGGCU | 4350 | AGCCTCCG GGCTAGCTACAACGA TTATGAGG | 9147 |
| 907 | AAGCGGAG G CUGGGAUG | 4351 | CATCCCAG GGCTAGCTACAACGA CTCCGCTT | 9148 |
| 901 | AGGCUGGG A UGGUCAGA | 4352 | TCTGACCA GGCTAGCTACAACGA CCCAGCCT | 9149 |
| 898 | CUGGGAUG G UCAGACAG | 4353 | CTGTCTGA GGCTAGCTACAACGA CATCCCAG | 9150 |
| 893 | AUGGUCAG A CAGGGCAG | 4354 | CTGCCCTG GGCTAGCTACAACGA CTGACCAT | 9151 |
| 888 | CAGACAGG G CAGCAGAG | 4355 | CTCTGCTG GGCTAGCTACAACGA CCTGTCTG | 9152 |
| 885 | ACAGGGCA G CAGAGCCA | 4356 | TGGCTCTG GGCTAGCTACAACGA TGCCCTGT | 9153 |
| 880 | GCAGCAGA G CCAAGAGG | 4357 | CCTCTTGG GGCTAGCTACAACGA TCTGCTGC | 9154 |
| 868 | AGAGGAAG A UAGAGAAA | 4358 | TTTCTCTA GGCTAGCTACAACGA CTTCCTCT | 9155 |
| 857 | GAGAAAGA G CAACCGGG | 4359 | CCCGGTTG GGCTAGCTACAACGA TCTTTCTC | 9156 |
| 854 | AAAGAGCA A CCGGGCAG | 4360 | CTGCCCGG GGCTAGCTACAACGA TGCTCTTT | 9157 |
| 849 | GCAACCGG G CAGAUUCC | 4361 | GGAATCTG GGCTAGCTACAACGA CCGGTTGC | 9158 |
| 845 | CCGGGCAG A UUCCCUGU | 4362 | ACAGGGAA GGCTAGCTACAACGA CTGCCCGG | 9159 |
| 838 | GAUUCCCU G UUGCAUAG | 4363 | CTATGCAA GGCTAGCTACAACGA AGGGAATC | 9160 |
| 835 | UCCCUGUU G CAUAGUUC | 4364 | GAACTATG GGCTAGCTACAACGA AACAGGGA | 9161 |
| 833 | CCUGUUGC A UAGUUCAC | 4365 | GTGAACTA GGCTAGCTACAACGA GCAACAGG | 9162 |
| 830 | GUUGCAUA G UUCACGCC | 4366 | GGCGTGAA GGCTAGCTACAACGA TATGCAAC | 9163 |
| 826 | CAUAGUUC A CGCCGUCU | 4367 | AGACGGCG GGCTAGCTACAACGA GAACTATG | 9164 |
| 824 | UAGUUCAC G CCGUCUUC | 4368 | GAAGACGG GGCTAGCTACAACGA GTGAACTA | 9165 |
| 821 | UUCACGCC G UCUUCCAG | 4369 | CTGGAAGA GGCTAGCTACAACGA GGCGTGAA | 9166 |
| 811 | CUUCCAGA A CCCGGACG | 4370 | CGTCCGGG GGCTAGCTACAACGA TCTGGAAG | 9167 |
| 805 | GAACCCGG A CGCCAUGC | 4371 | GCATGGCG GGCTAGCTACAACGA CCGGGTTC | 9168 |
| 803 | ACCCGGAC G CCAUGCGC | 4372 | GCGCATGG GGCTAGCTACAACGA GTCCGGGT | 9169 |
| 800 | CGGACGCC A UGCGCCAG | 4373 | CTGGCGCA GGCTAGCTACAACGA GGCGTCCG | 9170 |
| 798 | GACGCCAU G CGCCAGGG | 4374 | CCCTGGCG GGCTAGCTACAACGA ATGGCGTC | 9171 |
| 796 | CGCCAUGC G CCAGGGCC | 4375 | GGCCCTGG GGCTAGCTACAACGA GCATGGCG | 9172 |
| 790 | GCGCCAGG G CCCUGGCA | 4376 | TGCCAGGG GGCTAGCTACAACGA CCTGGCGC | 9173 |
| 784 | GGGCCCUG G CAGUGCCU | 4377 | AGGCACTG GGCTAGCTACAACGA CAGGGCCC | 9174 |
| 781 | CCCUGGCA G UGCCUCCC | 4378 | GGGAGGCA GGCTAGCTACAACGA TGCCAGGG | 9175 |
| 779 | CUGGCAGU G CCUCCCAA | 4379 | TTGGGAGG GGCTAGCTACAACGA ACTGCCAG | 9176 |
| 766 | CCAAGGGG G CGCCGACG | 4380 | CGTCGGCG GGCTAGCTACAACGA CCCCTTGG | 9177 |
| 764 | AAGGGGGC G CCGACGAG | 4381 | CTCGTCGG GGCTAGCTACAACGA GCCCCCTT | 9178 |
| 760 | GGGCGCCG A CGAGCGGA | 4382 | TCCGCTCG GGCTAGCTACAACGA CGGCGCCC | 9179 |
| 756 | GCCGACGA G CGGAAUGU | 4383 | ACATTCCG GGCTAGCTACAACGA TCGTCGGC | 9180 |
| 751 | CGAGCGGA A UGUACCCC | 4384 | GGGGTACA GGCTAGCTACAACGA TCCGCTCG | 9181 |
| 749 | AGCGGAAU G UACCCCAU | 4385 | ATGGGGTA GGCTAGCTACAACGA ATTCCGCT | 9182 |
| 747 | CGGAAUGU A CCCCAUGA | 4386 | TCATGGGG GGCTAGCTACAACGA ACATTCCG | 9183 |
| 742 | UGUACCCC A UGAGGUCG | 4387 | CGACCTCA GGCTAGCTACAACGA GGGGTACA | 9184 |
| 737 | CCCAUGAG G UCGGCGAA | 4388 | TTCGCCGA GGCTAGCTACAACGA CTCATGGG | 9185 |
| 733 | UGAGGUCG G CGAAGCCG | 4389 | CGGCTTCG GGCTAGCTACAACGA CGACCTCA | 9186 |
| 728 | UCGGCGAA G CCGCAUGU | 4390 | ACATGCGG GGCTAGCTACAACGA TTCGCCGA | 9187 |
| 725 | GCGAAGCC G CAUGUGAG | 4391 | CTCACATG GGCTAGCTACAACGA GGCTTCGC | 9188 |
| 723 | GAAGCCGC A UGUGAGGG | 4392 | CCCTCACA GGCTAGCTACAACGA GCGGCTTC | 9189 |
| 721 | AGCCGCAU G UGAGGGUA | 4393 | TACCCTCA GGCTAGCTACAACGA ATGCGGCT | 9190 |
| 715 | AUGUGAGG G UAUCGAUG | 4394 | CATCGATA GGCTAGCTACAACGA CCTCACAT | 9191 |
| 713 | GUGAGGGU A UCGAUGAC | 4395 | GTCATCGA GGCTAGCTACAACGA ACCCTCAC | 9192 |
| 709 | GGGUAUCG A UGACCUUA | 4396 | TAAGGTCA GGCTAGCTACAACGA CGATACCC | 9193 |
| 706 | UAUCGAUG A CCUUACCC | 4397 | GGGTAAGG GGCTAGCTACAACGA CATCGATA | 9194 |
| 701 | AUGACCUU A CCCAAGUU | 4398 | AACTTGGG GGCTAGCTACAACGA AAGGTCAT | 9195 |
| 695 | UUACCCAA G UUACGCGA | 4399 | TCGCGTAA GGCTAGCTACAACGA TTGGGTAA | 9196 |
| 692 | CCCAAGUU A CGCGACCU | 4400 | AGGTCGCG GGCTAGCTACAACGA AACTTGGG | 9197 |
| 690 | CAAGUUAC G CGACCUAC | 4401 | GTAGGTCG GGCTAGCTACAACGA GTAACTTG | 9198 |
| 687 | GUUACGCG A CCUACGCC | 4402 | GGCGTAGG GGCTAGCTACAACGA CGCGTAAC | 9199 |
| 683 | CGCGACCU A CGCCGGGG | 4403 | CCCCGGCG GGCTAGCTACAACGA AGGTCGCG | 9200 |
| 681 | CGACCUAC G CCGGGGGU | 4404 | ACCCCCGG GGCTAGCTACAACGA GTAGGTCG | 9201 |
| 674 | CGCCGGGG G UCCGUGGG | 4405 | CCCACGGA GGCTAGCTACAACGA CCCCGGCG | 9202 |
| 670 | GGGGGUCC G UGGGGCCC | 4406 | GGGCCCCA GGCTAGCTACAACGA GGACCCCC | 9203 |
| 665 | UCCGUGGG G CCCCAACU | 4407 | AGTTGGGG GGCTAGCTACAACGA CCCACGGA | 9204 |
| 659 | GGGCCCCA A CUAGGCCG | 4408 | CGGCCTAG GGCTAGCTACAACGA TGGGGCCC | 9205 |
| 654 | CCAACUAG G CCGGGAGC | 4409 | GCTCCCGG GGCTAGCTACAACGA CTAGTTGG | 9206 |
| 647 | GGCCGGGA G CCGCGGGG | 4410 | CCCCGCGG GGCTAGCTACAACGA TCCCGGCC | 9207 |
| 644 | CGGGAGCC G CGGGGUGA | 4411 | TCACCCCG GGCTAGCTACAACGA GGCTCCCG | 9208 |
| 639 | GCCGCGGG G UGACAGGA | 4412 | TCCTGTCA GGCTAGCTACAACGA CCCGCGGC | 9209 |
| 636 | GCGGGGUG A CAGGAGCC | 4413 | GGCTCCTG GGCTAGCTACAACGA CACCCCGC | 9210 |
| 630 | UGACAGGA G CCAUCCUG | 4414 | CAGGATGG GGCTAGCTACAACGA TCCTGTCA | 9211 |
| 627 | CAGGAGCC A UCCUGCCC | 4415 | GGGCAGGA GGCTAGCTACAACGA GGCTCCTG | 9212 |
| 622 | GCCAUCCU G CCCACCCU | 4416 | AGGGTGGG GGCTAGCTACAACGA AGGATGGC | 9213 |
| 618 | UCCUGCCC A CCCUAAGC | 4417 | GCTTAGGG GGCTAGCTACAACGA GGGCAGGA | 9214 |
| 611 | CACCCUAA G CCCUCAUU | 4418 | AATGAGGG GGCTAGCTACAACGA TTAGGGTG | 9215 |
| 605 | AAGCCCUC A UUGCCAUA | 4419 | TATGGCAA GGCTAGCTACAACGA GAGGGCTT | 9216 |
| 602 | CCCUCAUU G CCAUAGAG | 4420 | CTCTATGG GGCTAGCTACAACGA AATGAGGG | 9217 |
| 599 | UCAUUGCC A UAGAGGGG | 4421 | CCCCTCTA GGCTAGCTACAACGA GGCAATGA | 9218 |
| 591 | AUAGAGGG G CCAAGGGU | 4422 | ACCCTTGG GGCTAGCTACAACGA CCCTCTAT | 9219 |
| 584 | GGCCAAGG G UACCCGGG | 4423 | CCCGGGTA GGCTAGCTACAACGA CCTTGGCC | 9220 |
| 582 | CCAAGGGU A CCCGGGCU | 4424 | AGCCCGGG GGCTAGCTACAACGA ACCCTTGG | 9221 |
| 576 | GUACCCGG G CUGAGCCC | 4425 | GGGCTCAG GGCTAGCTACAACGA CCGGGTAC | 9222 |
| 571 | CGGGCUGA G CCCAGGCC | 4426 | GGCCTGGG GGCTAGCTACAACGA TCAGCCCG | 9223 |
| 565 | GAGCCCAG G CCCUGCCC | 4427 | GGGCAGGG GGCTAGCTACAACGA CTGGGCTC | 9224 |
| 560 | CAGGCCCU G CCCUCGGG | 4428 | CCCGAGGG GGCTAGCTACAACGA AGGGCCTG | 9225 |
| 552 | GCCCUCGG G CCGGCGAG | 4429 | CTCGCCGG GGCTAGCTACAACGA CCGAGGGC | 9226 |
| 548 | UCGGGCCG G CGAGCCUU | 4430 | AAGGCTCG GGCTAGCTACAACGA CGGCCCGA | 9227 |
| 544 | GCCGGCGA G CCUUGGGG | 4431 | CCCCAAGG GGCTAGCTACAACGA TCGCCGGC | 9228 |
| 535 | CCUUGGGG A UAGGUUGU | 4432 | ACAACCTA GGCTAGCTACAACGA CCCCAAGG | 9229 |
| 531 | GGGGAUAG G UUGUCGCC | 4433 | GGCGACAA GGCTAGCTACAACGA CTATCCCC | 9230 |
| 528 | GAUAGGUU G UCGCCUUC | 4434 | GAAGGCGA GGCTAGCTACAACGA AACCTATC | 9231 |
| 525 | AGGUUGUC G CCUUCCAC | 4435 | GTGGAAGG GGCTAGCTACAACGA GACAACCT | 9232 |
| 518 | CGCCUUCC A CGAGGUUG | 4436 | GAACCTCG GGCTAGCTACAACGA GGAAGGCG | 9233 |
| 513 | UCCACGAG G UUGCGACC | 4437 | GGTCGCAA GGCTAGCTACAACGA CTCGTGGA | 9234 |
| 510 | ACGAGGUU G CGACCGCU | 4438 | AGCGGTCG GGCTAGCTACAACGA AACCTCGT | 9235 |
| 507 | AGGUUGCG A CCGCUCGG | 4439 | CCGAGCCG GGCTAGCTACAACGA CGCAACCT | 9236 |
| 504 | UUGCGACC G CUCGGAAG | 4440 | CTTCCGAG GGCTAGCTACAACGA GGTCGCAA | 9237 |
| 496 | GCUCGGAA G UCUUCCUA | 4441 | TAGGAAGA GGCTAGCTACAACGA TTCCGAGC | 9238 |
| 487 | UCUUCCUA G UCGCGCGC | 4442 | GCGCGCGA GGCTAGCTACAACGA TAGGAAGA | 9239 |
| 484 | UCCAUGUC G CGCGCACA | 4443 | TGTGCGCG GGCTAGCTACAACGA GACTAGGA | 9240 |
| 482 | CUAGUCGC G CGCACACC | 4444 | GGTGTGCG GGCTAGCTACAACGA GCGACTAG | 9241 |
| 480 | AGUCGCGC G CACACCCA | 4445 | TGGGTGTG GGCTAGCTACAACGA GCGCGACT | 9242 |
| 478 | UCGCGCGC A CACCCAAC | 4446 | GTTGGGTG GGCTAGCTACAACGA GCGCGCGA | 9243 |
| 476 | GCGCGCAC A CCCAACCU | 4447 | AGGTTGGG GGCTAGCTACAACGA GTGCGCGC | 9244 |
| 471 | CACACCCA A CCUGGGGC | 4448 | GCCCCAGG GGCTAGCTACAACGA TGGGTGTG | 9245 |
| 464 | AACCUGGG G CCCCUGCG | 4449 | CGCAGGGG GGCTAGCTACAACGA CCCAGGTT | 9246 |
| 458 | GGGCCCCU G CGCGGCAA | 4450 | TTGCCGCG GGCTAGCTACAACGA AGGGGCCC | 9247 |
| 456 | GCCCCUGC G CGGCAACA | 4451 | TGTTGCCG GGCTAGCTACAACGA GCAGGGGC | 9248 |
| 453 | CCUGCGCG G CAACAGGU | 4452 | ACCTGTTG GGCTAGCTACAACGA CGCGCAGG | 9249 |
| 450 | GCGCGGCA A CAGGUAAA | 4453 | TTTACCTG GGCTAGCTACAACGA TGCCGCGC | 9250 |
| 446 | GGCAACAG G UAAACUCC | 4454 | GGAGTTTA GGCTAGCTACAACGA CTGTTGCC | 9251 |
| 442 | ACAGGUAA A CUCCACCA | 4455 | TGGTGGAG GGCTAGCTACAACGA TTACCTGT | 9252 |
| 437 | UAAACUCC A CCAACGAU | 4456 | ATCGTTGG GGCTAGCTACAACGA GGAGTTTA | 9253 |
| 433 | CUCCACCA A CGAUCUGA | 4457 | TCAGATCG GGCTAGCTACAACGA TGGTGGAG | 9254 |
| 430 | CACCAACG A UCUGACCA | 4458 | TGGTCAGA GGCTAGCTACAACGA CGTTGGTG | 9255 |
| 425 | ACGAUCUG A CCACCGCC | 4459 | GGCGGTGG GGCTAGCTACAACGA CAGATCGT | 9256 |
| 422 | AUCUGACC A CCGCCCGG | 4460 | CCGGGCGG GGCTAGCTACAACGA GGTCAGAT | 9257 |
| 419 | UGACCACC G CCCGGGAA | 4461 | TTCCCGGG GGCTAGCTACAACGA GGTGGTCA | 9258 |
| 411 | GCCCGGGA A CUUGACGU | 4462 | ACGTCAAG GGCTAGCTACAACGA TCCCGGGC | 9259 |
| 406 | GGAACUUG A CGUCCUGU | 4463 | ACAGGACG GGCTAGCTACAACGA CAAGTTCC | 9260 |
| 404 | AACUUGAC G UCCUGUGG | 4464 | CCACAGGA GGCTAGCTACAACGA GTCAAGTT | 9261 |
| 399 | GACGUCCU G UGGGCGGC | 4465 | GCCGCCCA GGCTAGCTACAACGA AGGACGTC | 9262 |
| 395 | UCCUGUGG G CGGCGGUU | 4466 | AACCGCCG GGCTAGCTACAACGA CCACAGGA | 9263 |
| 392 | UGUGGGCG G CGGUUGGU | 4467 | ACCAACCG GGCTAGCTACAACGA CGCCCACA | 9264 |
| 389 | GGGCGGCG G UUGGUGUU | 4468 | AACACCAA GGCTAGCTACAACGA CGCCGCCC | 9265 |
| 385 | GGCGGUUG G UGUUACGU | 4469 | ACGTAACA GGCTAGCTACAACGA CAACCGCC | 9266 |
| 383 | CGGUUGGU G UUACGUUU | 4470 | AAACGTAA GGCTAGCTACAACGA ACCAACCG | 9267 |
| 380 | UUGGUGUU A CGUUUGGU | 4471 | ACCAAACG GGCTAGCTACAACGA AACACCAA | 9268 |
| 378 | GGUGUUAC G UUUGGUUU | 4472 | AAACCAAA GGCTAGCTACAACGA GTAACACC | 9269 |
| 373 | UACGUUUG G UUUUUCUU | 4473 | AAGAAAAA GGCTAGCTACAACGA CAAACGTA | 9270 |
| 360 | UCUUUGAG G UUUAGGAU | 4474 | ATCCTAAA GGCTAGCTACAACGA CTCAAAGA | 9271 |
| 353 | GGUUUAGG A UUCGUGCU | 4475 | AGCACGAA GGCTAGCTACAACGA CCTAAACC | 9272 |
| 349 | UAGGAUUC G UGCUCAUG | 4476 | CATGAGCA GGCTAGCTACAACGA GAATCCTA | 9273 |
| 347 | GGAUUCGU G CUCAUGGU | 4477 | ACCATGAG GGCTAGCTACAACGA ACGAATCC | 9274 |
| 343 | UCGUGCUC A UGGUGCAC | 4478 | GTGCACCA GGCTAGCTACAACGA GAGCACGA | 9275 |
| 340 | UGCUCAUG G UGCACGGU | 4479 | ACCGTGCA GGCTAGCTACAACGA CATGAGCA | 9276 |
| 338 | CUCAUGGU G CACGGUCU | 4480 | AGACCGTG GGCTAGCTACAACGA ACCATGAG | 9277 |
| 336 | CAUGGUGC A CGGUCUAC | 4481 | GTAGACCG GGCTAGCTACAACGA GCACCATG | 9278 |
| 333 | GGUGCACG G UCUACGAG | 4482 | CTCGTAGA GGCTAGCTACAACGA CGTGCACC | 9279 |
| 329 | CACGGUCU A CGAGACCU | 4483 | AGGTCTCG GGCTAGCTACAACGA AGACCGTG | 9280 |
| 324 | UCUACGAG A CCUCCCGG | 4484 | CCGGGAGG GGCTAGCTACAACGA CTCGTAGA | 9281 |
| 314 | CUCCCGGG G CACUCGCA | 4485 | TGCGAGTG GGCTAGCTACAACGA CCCGGGAG | 9282 |
| 312 | CCCGGGGC A CUCGCAAG | 4486 | CTTGCGAG GGCTAGCTACAACGA GCCCCGGG | 9283 |
| 308 | GGGCACUC G CAAGCACC | 4487 | GGTGCTTG GGCTAGCTACAACGA GAGTGCCC | 9284 |
| 304 | ACUCGCAA G CACCCUAU | 4488 | ATAGGGTG GGCTAGCTACAACGA TTGCGAGT | 9285 |
| 302 | UCGCAAGC A CCCUAUCA | 4489 | TGATAGGG GGCTAGCTACAACGA GCTTGCGA | 9286 |
| 297 | AGCACCCU A UCAGGCAG | 4490 | CTGCCTGA GGCTAGCTACAACGA AGGGTGCT | 9287 |
| 292 | CCUAUCAG G CAGUACCA | 4491 | TGGTACTG GGCTAGCTACAACGA CTGATAGG | 9288 |
| 289 | AUCAGGCA G UACCACAA | 4492 | TTGTGGTA GGCTAGCTACAACGA TGCCTGAT | 9289 |
| 287 | CAGGCAGU A CCACAAGG | 4493 | CCTTGTGG GGCTAGCTACAACGA ACTGCCTG | 9290 |
| 284 | GCAGUACC A CAAGGCCU | 4494 | AGGCCTTG GGCTAGCTACAACGA GGTACTGC | 9291 |
| 279 | ACCACAAG G CCUUUCGC | 4495 | GCGAAAGG GGCTAGCTACAACGA CTTGTGGT | 9292 |
| 272 | GGCCUUUC G CGACCCAA | 4496 | TTGGGTCG GGCTAGCTACAACGA GAAAGGCC | 9293 |
| 269 | CUUUCGCG A CCCAACAC | 4497 | GTGTTGGG GGCTAGCTACAACGA CGCGAAAG | 9294 |
| 264 | GCGACCCA A CACUACUC | 4498 | GAGTAGTG GGCTAGCTACAACGA TGGGTCGC | 9295 |
| 262 | GACCCAAC A CUACUCGG | 4499 | CCGAGTAG GGCTAGCTACAACGA GTTGGGTC | 9296 |
| 259 | CCAACACU A CUCGGCUA | 4500 | TAGCCGAG GGCTAGCTACAACGA AGTGTTGG | 9297 |
| 254 | ACUACUCG G CUAGCAGU | 4501 | ACTGCTAG GGCTAGCTACAACGA CGAGTAGT | 9298 |
| 250 | CUCGGCUA G CAGUCUCG | 4502 | CGAGACTG GGCTAGCTACAACGA TAGCCGAG | 9299 |
| 247 | GGCUAGCA G UCUCGCGG | 4503 | CCGCGAGA GGCTAGCTACAACGA TGCTAGCC | 9300 |
| 242 | GCAGUCUC G CGGGGGCA | 4504 | TGCCCCCG GGCTAGCTACAACGA GAGACTGC | 9301 |
| 236 | UCGCGGGG G CACGCCCA | 4505 | TGGGCGTG GGCTAGCTACAACGA CCCCGCGA | 9302 |
| 234 | GCGGGGGC A CGCCCAAA | 4506 | TTTGGGCG GGCTAGCTACAACGA GCCCCCGC | 9303 |
| 232 | GGGGGCAC G CCCAAAUC | 4507 | GATTTGGG GGCTAGCTACAACGA GTGCCCCC | 9304 |
| 226 | ACGCCCAA A UCUCCAGG | 4508 | CCTGGAGA GGCTAGCTACAACGA TTGGGCGT | 9305 |
| 218 | AUCUCCAG G CAUUGAGC | 4509 | GCTCAATG GGCTAGCTACAACGA CTGGAGAT | 9306 |
| 216 | CUCCAGGC A UUGAGCGG | 4510 | CCGCTCAA GGCTAGCTACAACGA GCCTGGAG | 9307 |
| 211 | GGCAUUGA G CGGGUUGA | 4511 | TCAACCCG GGCTAGCTACAACGA TCAATGCC | 9308 |
| 207 | UUGAGCGG G UUGAUCCA | 4512 | TGGATCCA GGCTAGCTACAACGA CCGCTCAA | 9309 |
| 203 | GCGGGUUG A UCCAAGAA | 4513 | TTCTTGGA GGCTAGCTACAACGA CAACCCGC | 9310 |
| 191 | AAGAAAGG A CCCGGUCG | 4514 | CGACCGGG GGCTAGCTACAACGA CCTTTCTT | 9311 |
| 186 | AGGACCCG G UCGUCCUG | 4515 | CAGGACGA GGCTAGCTACAACGA CGGGTCCT | 9312 |
| 183 | ACCCGGUC G UCCUGGCA | 4516 | TGCCAGGA GGCTAGCTACAACGA GACCGGGT | 9313 |
| 177 | UCGUCCUG G CAAUUCCG | 4517 | CGGAATTG GGCTAGCTACAACGA CAGGACGA | 9314 |
| 174 | UCCUGGCA A UUCCGGUG | 4518 | CACCGGAA GGCTAGCTACAACGA TGCCAGGA | 9315 |
| 168 | CAAUUCCG G UGUACUCA | 4519 | TGAGTACA GGCTAGCTACAACGA CGGAATTG | 9316 |
| 166 | AUUCCGGU G UACUCACC | 4520 | GGTGAGTA GGCTAGCTACAACGA ACCGGAAT | 9317 |
| 164 | UCCGGUGU A CUCACCGG | 4521 | CCGGTGAG GGCTAGCTACAACGA ACACCGGA | 9318 |
| 160 | GUGUACUC A CCGGUUCC | 4522 | GGAACCGG GGCTAGCTACAACGA GAGTACAC | 9319 |
| 156 | ACUCACCG G UUCCGCAG | 4523 | CTGCGGAA GGCTAGCTACAACGA CGGTGAGT | 9320 |
| 151 | CCGGUUCC G CAGACCAC | 4524 | GTGGTCTG GGCTAGCTACAACGA GGAACCGG | 9321 |
| 147 | UUCCGCAG A CCACUAUG | 4525 | CATAGTGG GGCTAGCTACAACGA CTGCGGAA | 9322 |
| 144 | CGCAGACC A CUAUGGCU | 4526 | AGCCATAG GGCTAGCTACAACGA GGTCTGCG | 9323 |
| 141 | AGACCACU A UGGCUCUC | 4527 | GAGAGCCA GGCTAGCTACAACGA AGTGGTCT | 9324 |
| 138 | CCACUAUG G CUCUCCCG | 4528 | CGGGAGAG GGCTAGCTACAACGA CATAGTGG | 9325 |
| 120 | GAGGGGGG G UCCUGGAG | 4529 | CTCCAGGA GGCTAGCTACAACGA CCCCCCTC | 9326 |
| 111 | UCCUGGAG G CUGCACGA | 4530 | TCGTGCAG GGCTAGCTACAACGA CTCCAGGA | 9327 |
| 108 | UGGAGGCU G CACGACAC | 4531 | GTGTCGTG GGCTAGCTACAACGA AGCCTCCA | 9328 |
| 106 | GAGGCUGC A CGACACUC | 4532 | GAGTGTCG GGCTAGCTACAACGA GCAGCCTC | 9329 |
| 103 | GCUGCACG A CACUCAUA | 4533 | TATGAGTG GGCTAGCTACAACGA CGTGCAGC | 9330 |
| 101 | UGCACGAC A CUCAUACU | 4534 | AGTATGAG GGCTAGCTACAACGA GTCGTGCA | 9331 |
| 97 | CGACACUC A UACUAACG | 4535 | CGTTAGTA GGCTAGCTACAACGA GAGTGTCG | 9332 |
| 95 | ACACUCAU A CUAACGCC | 4536 | GGCGTTAG GGCTAGCTACAACGA ATGAGTGT | 9333 |
| 91 | UCAUACUA A CGCCAUGG | 4537 | CCATGGCG GGCTAGCTACAACGA TAGTATGA | 9334 |
| 89 | AUACUAAC G CCAUGGCU | 4538 | AGCCATGG GGCTAGCTACAACGA GTTAGTAT | 9335 |
| 86 | CUAACGCC A UGGCUAGA | 4539 | TCTAGCCA GGCTAGCTACAACGA GGCGTTAG | 9336 |
| 83 | ACGCCAUG G CUAGACGC | 4540 | GCGTCTAG GGCTAGCTACAACGA CATGGCGT | 9337 |
| 78 | AUGGCUAG A CGCUUUCU | 4541 | AGAAAGCG GGCTAGCTACAACGA CTAGCCAT | 9338 |
| 76 | GGCUAGAC G CUUUCUGC | 4542 | GCAGAAAG GGCTAGCTACAACGA GTCTAGCC | 9339 |
| 69 | CGCUUUCU G CGUGAAGA | 4543 | TCTTCACG GGCTAGCTACAACGA AGAAAGCG | 9340 |
| 67 | CUUUCUGC G UGAAGACA | 4544 | TGTCTTCA GGCTAGCTACAACGA GCAGAAAG | 9341 |
| 61 | GCGUGAAG A CAGUAGUU | 4545 | AACTACTG GGCTAGCTACAACGA CTTCACGC | 9342 |
| 58 | UGAAGACA G UAGUUCCU | 4546 | AGGAACTA GGCTAGCTACAACGA TGTCTTCA | 9343 |
| 55 | AGACAGUA G UUCCUCAC | 4547 | GTGAGGAA GGCTAGCTACAACGA TACTGTCT | 9344 |
| 48 | AGUUCCUC A CAGGGGAG | 4548 | CTCCCCTG GGCTAGCTACAACGA GAGGAACT | 9345 |
| 40 | ACAGGGGA G UGAUCUAU | 4549 | ATAGATCA GGCTAGCTACAACGA TCCCCTGT | 9346 |
| 37 | GGGGAGUG A UCUAUGGU | 4550 | ACCATAGA GGCTAGCTACAACGA CACTCCCC | 9347 |
| 33 | AGUGAUCU A UGGUGGAG | 4551 | CTCCACCA GGCTAGCTACAACGA AGATCACT | 9348 |
| 30 | GAUCUAUG G UGGAGUGU | 4552 | ACACTCCA GGCTAGCTACAACGA CATAGATC | 9349 |
| 25 | AUGGUGGA G UGUCGCCC | 4553 | GGGCGACA GGCTAGCTACAACGA TCCACCAT | 9350 |
| 23 | GGUGGAGU G UCGCCCCC | 4554 | GGGGGCGA GGCTAGCTACAACGA ACTCCACC | 9351 |

Input Sequence = HPCK1S1.
Cut Site = R/Y
Arm Length = 8.
Core Sequence = GGCTAGCTACAACGA
HPCK1S1 Hepatitis C virus (strain HCV-1b, clone HCV-K1-S1), complete genome; acc# gi|1030702|dbj|D50483.1; 9410 nt

TABLE V
Synthetic anti-HCV nucleic acid molecule and Target Sequences

| | | | | | | | Nu- |
|---|---|---|---|---|---|---|---|
| | | | | | | | cleic |
| ref | Ref | | Seq | | | Seq | Acid |
| pos | Seq | Target | ID | RPI# | Nucleic Acid | ID | Alias |
| 195 | HCV+ | GGGUCCU U UCUUGGA | 4556 | 15364 | cₛcₛaₛaₛga cUGAuGaggcgaaagccGaa Aggacc B | 9352 | Ham- |
| | | | | | | | mer- |
| | | | | | | | head |
| 342 | HCV+ | AGACCGUGCAUCAUGAGCAC | 4555 | 17501 | GₛTₛGₛCₛTₛCₛAₛTₛGₛAₛTₛGₛCₛAₛCₛGₛGₛTₛCₛT | 9353 | Anti- |
| | | | | | | | sense |
| 195 | HCV+ | GGGUCCU U UCUUGGA | 4556 | 17558 | cₛcₛaₛaₛga cUGAuGaggcguuagccGaZ Aggacc B | 9354 | Ham- |
| | | | | | | | mer- |
| | | | | | | | head |
| 195 | HCV+ | GGGUCCU U UCUUGGA | 4556 | 17559 | cₛcₛaₛaₛga cUGAuGaggcguuagccGaa AggaZc B | 9355 | Ham- |
| | | | | | | | mer- |
| | | | | | | | head |
| 195 | HCV+ | GGGUCCU U UCUUGGA | 4556 | 17560 | Zₛcₛaₛaₛga cUGAuGaggcguuagccGaa Aggacc B | 9356 | Ham- |
| | | | | | | | mer- |
| | | | | | | | head |
| 195 | HCV+ | GGGUCCU U UCUUGGA | 4556 | 17561 | Z cₛaₛaₛga cUGAuGaggcguuagccGaa Aggacc B | 9357 | Ham- |
| | | | | | | | mer- |
| | | | | | | | head |
| 195 | HCV+ | GGGUCCU U UCUUGGA | 4556 | 18012 | ccaagacUGAuGaggcguuagccGaa Aggacc B | 9358 | Ham- |
| | | | | | | | mer- |
| | | | | | | | head |
| 82 | HCV+ | GCGUCUA G CCAUGGC | 4557 | 18744 | gₛcₛcₛaₛugg GccgaaagGCGaGucaaGGuCu uagacgc B | 9359 | Zin- |
| | | | | | | | zyme |
| 100 | HCV+ | AGUAUGA G UGUCGUG | 4558 | 18745 | cₛaₛcₛgₛaca GccgaaagGCGaGucaaGGuCu ucauacu B | 9360 | Zin- |
| | | | | | | | zyme |
| 102 | HCV+ | UAUGAGU G UCGUGCA | 4559 | 18746 | uₛgₛcₛaₛcga GccgaaagGCGaGucaaGGuCu acucaua B | 9361 | Zin- |
| | | | | | | | zyme |
| 105 | HCV+ | GAGUGUC G UGCAGCC | 4560 | 18747 | gₛgₛcₛuₛgca GccgaaagGCGaGucaaGGuCu gacacuc B | 9362 | Zin- |
| | | | | | | | zyme |
| 107 | HCV+ | GUGUCGU G CAGCCUC | 4561 | 18748 | gₛaₛgₛgₛcug GccgaaagGCGaGucaaGGuCu acgacac B | 9363 | Zin- |
| | | | | | | | zyme |
| 146 | HCV+ | CAUAGUG G UCUGCGG | 4562 | 18749 | cₛcₛgₛcₛaga GccgaaagGCGaGucaaGGuCu cacuaug B | 9364 | Zin- |
| | | | | | | | zyme |
| 190 | HCV+ | CGACCGG G UCCUUUC | 4563 | 18750 | gₛaₛaₛaₛgga GccgaaagGCGaGucaaGGuCu ccggucg B | 9365 | Zin- |
| | | | | | | | zyme |
| 217 | HCV+ | GCUCAAU G CCUGGAG | 4564 | 18751 | cₛuₛcₛcₛagg GccgaaagGCGaGucaaGGuCu auugagc B | 9366 | Zin- |
| | | | | | | | zyme |
| 231 | HCV+ | GAUUUGG G CGUGCCC | 4565 | 18752 | gₛgₛgₛcₛacg GccgaaagGCGaGucaaGGuCu ccaaauc B | 9367 | Zin- |
| | | | | | | | zyme |
| 258 | HCV+ | UAGCCGA G UAGUGUU | 4566 | 18753 | aₛaₛcₛaₛcua GccgaaagGCGaGucaaGGuCu ucggcua B | 9368 | Zin- |
| | | | | | | | zyme |
| 307 | HCV+ | GGUGCUU G CGAGUGC | 4567 | 18754 | gₛcₛaₛcₛucg GccgaaagGCGaGucaaGGuCu aagcacc B | 9369 | Zin- |
| | | | | | | | zyme |
| 77 | HCV+ | GAAAGC G UCUAGC | 4568 | 18755 | gₛcₛuₛaₛga GccgaaagGCGaGucaaGGuCu gcuuuc B | 9370 | Zin- |
| | | | | | | | zyme |
| 77 | HCV+ | AGAAAGC G UCUAGCC | 4569 | 18756 | gₛgₛcₛuₛaga GccgaaagGCGaGucaaGGuCu gcuuucu B | 9371 | Zin- |
| | | | | | | | zyme |
| 88 | HCV+ | AGCCAUG G CGUUAGU | 4570 | 18757 | aₛcₛuₛaₛacg GccgaaagGCGaGucaaGGuCu cauggcu B | 9372 | Zin- |
| | | | | | | | zyme |
| 94 | HCV+ | GCGUUA G UAUGAGU | 4571 | 18758 | aₛcₛuₛcₛaua GccgaaagGCGaGucaaGGuCu uaacgcc B | 9373 | Zin- |
| | | | | | | | zyme |
| 102 | HCV+ | AUGAGU G UCGUGC | 4572 | 18759 | gₛcₛaₛcₛga GccgaaagGCGaGucaaGGuCu acucau B | 9374 | Zin- |
| | | | | | | | zyme |
| 105 | HCV+ | AGUGUC G UGCAGC | 4573 | 18760 | gₛcₛuₛgₛca GccgaaagGCGaGucaaGGuCu gacacu B | 9375 | Zin- |
| | | | | | | | zyme |
| 110 | HCV+ | UCGUGCA G CCUCCAG | 4574 | 18761 | cₛuₛgₛgₛagg GccgaaagGCGaGucaaGGuCu ugcacga B | 9376 | Zin- |
| | | | | | | | zyme |
| 137 | HCV+ | GGGAGA G CCAUAG | 4575 | 18762 | cₛuₛaₛuₛgg GccgaaagGCGaGucaaGGuCu ucuccc B | 9377 | Zin- |
| | | | | | | | zyme |
| 137 | HCV+ | CGGGAGA G CCAUAGU | 4576 | 18763 | aₛcₛuₛaₛugg GccgaaagGCGaGucaaGGuCu ucucccg B | 9378 | Zin- |
| | | | | | | | zyme |
| 146 | HCV+ | AUAGUG G UCUGCG | 4577 | 18764 | cₛgₛcₛaₛga GccgaaagGCGaGucaaGGuCu cacuau B | 9379 | Zin- |
| | | | | | | | zyme |
| 150 | HCV+ | GUGGUCU G CGGAACC | 4578 | 18765 | gₛgₛuₛuₛccg GccgaaagGCGaGucaaGGuCu agaccac B | 9380 | Zin- |
| | | | | | | | zyme |
| 176 | HCV+ | CGGAAUU G CCAGGAC | 4579 | 18766 | gₛuₛcₛcₛugg GccgaaagGCGaGucaaGGuCu aauuccg B | 9381 | Zin- |
| | | | | | | | zyme |
| 190 | HCV+ | GACCGG G UCCUUU | 4580 | 18767 | aₛaₛaₛgₛga GccgaaagGCGaGucaaGGuCu ccgguc B | 9382 | Zin- |
| | | | | | | | zyme |
| 253 | HCV+ | CUGCUA G CCGAGU | 4581 | 18768 | aₛcₛuₛcₛgg GccgaaagGCGaGuoaaGGuCu uagcag B | 9383 | Zin- |
| | | | | | | | zyme |
| 253 | HCV+ | ACUGCUA G CCGAGUA | 4582 | 18769 | uₛaₛcₛuₛcgg GccgaaagGCGaGucaaGGuCu uagcagu B | 9384 | Zin- |
| | | | | | | | zyme |
| 258 | HCV+ | AGCCGA G UAGUGU | 4583 | 18770 | aₛcₛaₛcₛua GccgaaagGCGaGucaaGGuCu ucggcu B | 9385 | Zin- |
| | | | | | | | zyme |
| 263 | HCV+ | GAGUAGU G UUGGGUC | 4584 | 18771 | gₛaₛcₛcₛcaa GccgaaagGCGaGucaaGGuCu acuacuc B | 9386 | Zin- |
| | | | | | | | zyme |
| 268 | HCV+ | UGUUGG G UCGCGA | 4585 | 18772 | uₛcₛgₛcₛga GccgaaagGCGaGucaaGGuCu ccaaca B | 9387 | Zin- |
| | | | | | | | zyme |
| 268 | HCV+ | GUGUUGG G UCGCGAA | 4586 | 18773 | uₛuₛcₛgₛcga GccgaaagGCGaGucaaGGuCu ccaacac B | 9388 | Zin- |
| | | | | | | | zyme |
| 271 | HCV+ | UUGGGUC G CGAAAGG | 4587 | 18774 | cₛcₛuₛuₛucg GccgaaagGCGaGucaaGGuCu gacccaa B | 9389 | Zin- |
| | | | | | | | zyme |
| 283 | HCV+ | AGGCCUU G UGGUACU | 4588 | 18775 | aₛgₛuₛaₛcca GccgaaagGCGaGucaaGGuCu aaggccu B | 9390 | Zin- |
| | | | | | | | zyme |
| 286 | HCV+ | CCUUGUG G UACUGCC | 4589 | 18776 | gₛgₛcₛaₛgua GccgaaagGCGaGucaaGGuCu cacaagg B | 9391 | Zin- |
| | | | | | | | zyme |
| 291 | HCV+ | UGGUACU G CCUGAUA | 4590 | 18777 | uₛaₛuₛcₛagg GccgaaagGCGaGucaaGGuCu aguacca B | 9392 | Zin- |
| | | | | | | | zyme |
| 301 | HCV+ | UGAUAGG G UGCUUGC | 4591 | 18778 | gₛcₛaₛaₛgca GccgaaagGCGaGucaaGGuCu ccuauca B | 9393 | Zin- |
| | | | | | | | zyme |
| 303 | HCV+ | AUAGGGU G CUUGCGA | 4592 | 18779 | uₛcₛgₛcₛaag GccgaaagGCGaGucaaGGuCu acccuau B | 9394 | Zin- |
| | | | | | | | zyme |
| 60 | HCV+ | ACUACU G UCUUCA | 4593 | 18780 | uₛgₛaₛaₛga GccgaaagGCGaGucaaGGuCu aguagu B | 9395 | Zin- |
| | | | | | | | zyme |
| 60 | HCV+ | AACUACU G UCUUCAC | 4594 | 18781 | gₛuₛgₛaₛaga GccgaaagGCGaGucaaGGuCu augaguu B | 9396 | Zin- |
| | | | | | | | zyme |
| 68 | HCV+ | UCUUCAC G CAGAAAG | 4595 | 18782 | cₛuₛuₛuₛcug GccgaaagGCGaGucaaGGuCu gugaaga B | 9397 | Zin- |
| | | | | | | | zyme |
| 75 | HCV+ | CAGAAA G CGUCUA | 4596 | 18783 | uₛaₛgₛaₛcg GccgaaagGCGaGucaaGGuCu uuucug B | 9398 | Zin- |
| | | | | | | | zyme |
| 82 | HCV+ | CGUCUA G CCAUGG | 4597 | 18784 | cₛcₛaₛuₛgg GccgaaagGCGaGucaaGGuCu uagacg B | 9399 | Zin- |
| | | | | | | | zyme |
| 88 | HCV+ | GCCAUG G CGUUAG | 4598 | 18785 | cₛuₛaₛaₛcg GccgaaagGCGaGucaaGGuCu cauggc B | 9400 | Zin- |
| | | | | | | | zyme |
| 90 | HCV+ | CAUGGC G UUAGUA | 4599 | 18786 | uₛaₛcₛuₛaa GccgaaagGCGaGucaaGCuCu gccaug B | 9401 | Zin- |
| | | | | | | | zyme |
| 90 | HCV+ | CCAUGGC G UUAGUAU | 4600 | 18787 | aₛuₛaₛcₛuaa GccgaaagGCGaGucaaGGuCu gccaugg B | 9402 | Zin- |
| | | | | | | | zyme |
| 100 | HCV+ | GUAUGA G UGUCGU | 4601 | 18788 | aₛcₛgₛaₛca GccgaaagGCGaGucaaGGuCu ucauac B | 9403 | Zin- |
| | | | | | | | zyme |
| 107 | HCV+ | UGUCGU G CAGCCU | 4602 | 18789 | aₛgₛgₛcₛug GccgaaagGCGaGucaaGGuCu acgaca B | 9404 | Zin- |
| | | | | | | | zyme |
| 110 | HCV+ | CGUCCA G CCUCCA | 4603 | 18790 | uₛgₛgₛaₛgg GccgaaagGCGaGucaaGGuCu acgaca B | 9405 | Zin- |
| | | | | | | | zyme |
| 150 | HCV+ | UGGUCU G CGGAAC | 4604 | 18791 | gₛuₛuₛcₛcg GccgaaagGCGaGucaaGGuCu ugcacg B | 9406 | Zin- |
| | | | | | | | zyme |
| 159 | HCV+ | GGAACCG G UGAGUAC | 4605 | 18792 | gₛuₛaₛcₛuca GccgaaagGCGaGucaaGGuCu cgguucc B | 9407 | Zin- |
| | | | | | | | zyme |
| 176 | HCV+ | GGAAUU G CCAGGA | 4606 | 18793 | uₛcₛcₛuₛgg GccgaaagGCGaGucaaGGuCu aauucc B | 9408 | Zin- |
| | | | | | | | zyme |
| 217 | HCV+ | CUCAAU G CCUGGA | 4607 | 18794 | uₛcₛcₛaₛgg GccgaaagGCGaGucaaGGuCu auugag B | 9409 | Zin- |
| | | | | | | | zyme |
| 231 | HCV+ | AUUUGG G CGUGCC | 4608 | 18795 | gₛgₛcₛaₛcg GccgaaagGCGaGucaaGGuCu ccaaau B | 9410 | Zin- |
| | | | | | | | zyme |
| 261 | HCV+ | CGAGUA G UGUUGG | 4609 | 18796 | cₛcₛaₛaₛca GccgaaagGCGaGucaaGGuCu uacucg B | 9411 | Zin- |
| | | | | | | | zyme |
| 261 | HCV+ | CCGAGUA G UGUUGGG | 4610 | 18797 | cₛcₛcₛaₛaca GccgaaagGCGaGucaaGGuCu uacucgg B | 9412 | Zin- |
| | | | | | | | zyme |
| 263 | HCV+ | AGUAGU G UUGGGU | 4611 | 18798 | aₛcₛcₛcₛaa GccgaaagGCGaGucaaGGuCu acuacu B | 9413 | Zin- |
| | | | | | | | zyme |
| 271 | HCV+ | UGGGUC G CGAAAG | 4612 | 18799 | cₛuₛuₛuₛcg GccgaaagGCGaGucaaGGuCu gaccca B | 9414 | Zin- |
| | | | | | | | zyme |
| 283 | HCV+ | GGCCUU G UGGUAC | 4613 | 18800 | gₛuₛaₛcₛca GccgaaagGCGaGucaaGGuCu aaggcc B | 9415 | Zin- |
| | | | | | | | zyme |
| 291 | HCV+ | GGUACU G CCUGAU | 4614 | 18801 | aₛuₛcₛaₛgg GccgaaagGCGaGucaaGGuCu aguacc B | 9416 | Zin- |
| | | | | | | | zyme |
| 303 | HCV+ | UAGGGU G CUUGCG | 4615 | 18802 | cₛgₛcₛaₛag GccgaaagGCGaGucaaGGuCu acccua B | 9417 | Zin- |
| | | | | | | | zyme |
| 307 | HCV+ | GUGCUU G CGAGUG | 4616 | 18803 | cₛaₛcₛuₛcg GccgaaagGCGaGucaaGGuCu aagcac B | 9418 | Zin- |
| | | | | | | | zyme |
| 323 | HCV+ | CGGGAG G UCUCGU | 4617 | 18804 | aₛcₛgₛaₛga GccgaaagGCGaGucaaGGuCu cucccg B | 9419 | Zin- |
| | | | | | | | zyme |
| 323 | HCV+ | CCGGGAG G UCUCGUA | 4618 | 18805 | uₛaₛcₛgₛaga GccgaaagGCGaGucaaGGuCu cucccgg B | 9420 | Zin- |
| | | | | | | | zyme |
| 75 | HCV+ | GCAGAAA G CGUCUAG | 4619 | 18806 | cₛuₛaₛgₛacg GccgaaagGCGaGucaaGGuCu uuucugc B | 9421 | Zin- |
| | | | | | | | zyme |
| 143 | HCV+ | GCCAUA G UGGUCU | 4620 | 18807 | aₛgₛaₛcₛca GccgaaagGCGaGucaaGGuCu uauggc B | 9422 | Zin- |
| | | | | | | | zyme |
| 278 | HCV+ | GCGAAAG G CCUUGUG | 4621 | 18808 | cₛaₛcₛaₛagg GccgaaagGCGaGucaaGGuCu cuuucgc B | 9423 | Zin- |
| | | | | | | | zyme |
| 163 | HCV+ | CGGUGA G UACACC | 4622 | 18809 | gₛgₛuₛgₛua GccgaaagGCGaGucaaGGuCu ucaccg B | 9424 | Zin- |
| | | | | | | | zyme |
| 68 | HCV+ | CUUCAC G CAGAAA | 4623 | 18810 | uₛuₛuₛcₛug GccgaaagGCGaGucaaGGuCu gugaag B | 9425 | Zin- |
| | | | | | | | zyme |
| 94 | HCV+ | GCGUUA G UAUGAG | 4624 | 18811 | cₛuₛcₛaₛua GccgaaagGCGaGucaaGGuCu uuaacgc B | 9426 | Zin- |
| | | | | | | | zyme |
| 143 | HCV+ | AGCCAUA G UGGUCUG | 4625 | 18812 | cₛaₛgₛaₛcca GccgaaagGCGaGucaaGGuCu uauggcu B | 9427 | Zin- |
| | | | | | | | zyme |
| 159 | HCV+ | GAACCG G UGAGUA | 4626 | 18813 | uₛaₛcₛuₛca GccgaaagGCGaGucaaGGuCu cgguuc B | 9428 | Zin- |
| | | | | | | | zyme |
| 163 | HCV+ | CCGGUGA G UACACCG | 4627 | 18814 | cₛgₛgₛuₛgua GccgaaagGCGaGucaaGGuCu ucaccgg B | 9429 | Zin- |
| | | | | | | | zyme |
| 249 | HCV+ | GAGACU G CUAGCC | 4628 | 18815 | gₛgₛcₛuₛag GccgaaagGCGaGucaaGGuCu agucuc B | 9430 | Zin- |
| | | | | | | | zyme |
| 249 | HCV+ | CGAGACU G CUAGCCG | 4629 | 18816 | cₛgₛgₛcₛuag GcogaaagGCGaGucaaGGuCu agucucg B | 9431 | Zin- |
| | | | | | | | zyme |
| 278 | HCV+ | CGAAAG G CCUUGU | 4630 | 18817 | aₛcₛaₛaₛgg GccgaaagGCGaGucaaGGuCu cuuucg B | 9432 | Zin- |
| | | | | | | | zyme |
| 286 | HCV+ | CUUGUG G UACUGC | 4631 | 18818 | gₛcₛaₛgₛua GccgaaagGCGaGucaaGGuCu cacaag B | 9433 | Zin- |
| | | | | | | | zyme |
| 301 | HCV+ | GAUAGG G UGCUUG | 4632 | 18819 | cₛaₛaₛgₛca GccgaaagGCGaGucaaGGuCu ccuauc B | 9434 | Zin- |
| | | | | | | | zyme |
| 328 | HCV+ | GGUCUC G UAGACC | 4633 | 18820 | gₛgₛuₛcₛua GccgaaagGCGaGucaaGGuCu gagacc B | 9435 | Zin- |
| | | | | | | | zyme |
| 328 | HCV+ | AGGUCUC G UAGACCG | 4634 | 18821 | cₛgₛgₛuₛcua GccgaaagGCGaGucaaGGuCu gagaccu B | 9436 | Zin- |
| | | | | | | | zyme |
| 335 | HCV+ | UAGACC G UGCACC | 4635 | 18822 | gₛgₛuₛgₛca GccgaaagGCGaGucaaGGuCu ggucua B | 9437 | Zin- |
| | | | | | | | zyme |
| 30 | C | UAAACCU C AAAGAAA | 4636 | 19108 | uₛuₛuₛcₛuuu cUGAuGaggccguuaggccGaa Agguuua B | 9438 | Ham- |
| | | | | | | | mer- |
| | | | | | | | head |
| 48 | C | CAAACGU A ACACCAA | 4637 | 19109 | uₛuₛgₛgₛugu cUGAuGaggccguuaggccGaa Acguuug B | 9439 | Ham- |
| | | | | | | | mer- |
| | | | | | | | head |
| 60 | C | CAACCGU C GCCCACA | 4638 | 19110 | uₛgₛuₛgₛggc cUGAuGaggccguuaggccGaa Acgguug B | 9440 | Ham- |
| | | | | | | | mer- |
| | | | | | | | head |
| 175 | C | GAGCGGU C ACAACCU | 4639 | 19111 | aₛgₛgₛuₛugu cUGAuGaggccguuaggccGaa Accgcuc B | 9441 | Ham- |
| | | | | | | | mer- |
| | | | | | | | head |
| 374 | C | GUAAGGU C AUCGAUA | 4640 | 19112 | uₛaₛuₛcₛgau cUGAuGaggccguuaggccGaa Accuuac B | 9442 | Ham- |
| | | | | | | | mer- |
| | | | | | | | head |
| 258 | S27 | UGGUGGCUCCAUCUUAGCCCUAG | 4641 | 22022 | uₛgₛgₛuₛgₛgₛcₛuₛcₛcₛaₛuₛcₛuₛuₛaₛgₛcₛcₛcₛuₛaₛg | 9443 | Anti- |
| | | | | | | | sense |
| 259 | S27 | GGUGGCUCCAUCUUAGCCCUAGU | 4642 | 22023 | gₛgₛuₛgₛgₛcₛuₛcₛcₛaₛuₛcₛuₛuₛaₛgₛcₛcₛcₛuₛaₛgₛu | 9444 | Anti- |
| | | | | | | | sense |
| 260 | S27 | GUGGCUCCAUCUUAGCCCUAGUC | 4643 | 22024 | gₛuₛgₛgₛcₛuₛcₛcₛaₛuₛcₛuₛuₛaₛgₛcₛcₛcₛuₛaₛgₛuₛc | 9445 | Anti- |
| | | | | | | | sense |
| 261 | S27 | UGGCUCCAUCUUAGCCCUAGUCA | 4644 | 22025 | uₛgₛgₛcₛuₛcₛcₛaₛuₛcₛuₛuₛaₛgₛcₛcₛcₛuₛaₛgₛuₛcₛa | 9446 | Anti- |
| | | | | | | | sense |
| 262 | S27 | GGCUCCAUCUUAGCCCUAGUCAC | 4645 | 22026 | gₛgₛcₛuₛcₛcₛaₛuₛcₛuₛuₛaₛgₛcₛcₛcₛuₛaₛgₛuₛcₛaₛc | 9447 | Anti- |
| | | | | | | | sense |
| 263 | S27 | GCUCCAUCUUAGCCCUAGUCACG | 4646 | 22027 | gₛcₛuₛcₛcₛaₛuₛcₛuₛuₛaₛgₛcₛcₛcₛuₛaₛgₛuₛcₛaₛcₛg | 9448 | Anti- |
| | | | | | | | sense |
| 264 | S27 | CUCCAUCUUAGCCCUAGUCACGG | 4647 | 22028 | cₛuₛcₛcₛaₛuₛcₛuₛuₛaₛgₛcₛcₛcₛuₛaₛgₛuₛcₛaₛcₛgₛg | 9449 | Anti- |
| | | | | | | | sense |
| 265 | S27 | UCCAUCUUAGCCCUAGUCACGGC | 4648 | 22029 | uₛcₛcₛaₛuₛcₛuₛuₛaₛgₛcₛcₛcₛuₛaₛgₛuₛcₛaₛcₛgₛgₛc | 9450 | Anti- |
| | | | | | | | sense |
| 266 | S27 | CCAUCUUAGCCCUAGUCACGGCU | 4649 | 22030 | cₛcₛaₛuₛcₛuₛuₛaₛgₛcₛcₛcₛuₛaₛgₛuₛcₛaₛcₛgₛgₛcₛu | 9451 | Anti- |
| | | | | | | | sense |
| 267 | S27 | CAUCUUAGCCCUAGUCACGGCUA | 4650 | 22031 | cₛaₛuₛcₛuₛuₛaₛgₛcₛcₛcₛuₛaₛgₛuₛcₛaₛcₛgₛgₛcₛuₛa | 9452 | Anti- |
| | | | | | | | sense |
| 268 | S27 | AUCUUAGCCCUAGUCACGGCUAG | 4651 | 22032 | aₛuₛcₛuₛuₛaₛgₛcₛcₛcₛuₛaₛgₛuₛcₛaₛcₛgₛgₛcₛuₛaₛg | 9453 | Anti- |
| | | | | | | | sense |
| 269 | S27 | UCUUAGCCCUAGUCACGGCUAGC | 4652 | 22033 | uₛcₛuₛuₛaₛgₛcₛcₛcₛuₛaₛgₛuₛcₛaₛcₛgₛgₛcₛuₛaₛgₛc | 9454 | Anti- |
| | | | | | | | sense |
| 270 | S27 | CUUAGCCCUAGUCACGGCUAGCU | 4653 | 22034 | cₛuₛuₛaₛgₛcₛcₛcₛuₛaₛgₛuₛcₛaₛcₛgₛgₛcₛuₛaₛgₛcₛu | 9455 | Anti- |
| | | | | | | | sense |
| 271 | S27 | UUAGCCCUAGUCACGGCUAGCUG | 4654 | 22035 | uₛuₛaₛgₛcₛcₛcₛuₛaₛgₛuₛcₛaₛcₛgₛgₛcₛuₛaₛgₛcₛuₛg | 9456 | Anti- |
| | | | | | | | sense |
| 272 | S27 | UAGCCCUAGUCACGGCUAGCUGU | 4655 | 22036 | uₛaₛgₛcₛcₛcₛuₛaₛgₛuₛcₛaₛcₛgₛgₛcₛuₛaₛgₛcₛuₛgₛu | 9457 | Anti- |
| | | | | | | | sense |
| 273 | S27 | AGCCCUAGUCACGGCUAGCUGUG | 4656 | 22037 | aₛgₛcₛcₛcₛuₛaₛgₛuₛcₛaₛcₛgₛgₛcₛuₛaₛgₛcₛuₛgₛuₛg | 9458 | Anti- |
| | | | | | | | sense |
| 274 | S27 | GCCCUAGUCACGGCUAGCUGUGA | 4657 | 22038 | gₛcₛcₛcₛuₛaₛgₛuₛcₛaₛcₛgₛgₛcₛuₛaₛgₛcₛuₛgₛuₛgₛa | 9459 | Anti- |
| | | | | | | | sense |
| 275 | S27 | CCCUAGUCACGGCUAGCUGUGAA | 4658 | 22039 | cₛcₛcₛuₛaₛgₛuₛcₛaₛcₛgₛgₛcₛuₛaₛgₛcₛuₛgₛuₛgₛaₛa | 9460 | Anti- |
| | | | | | | | sense |
| 276 | S27 | CCUAGUCACGGCUAGCUGUGAAA | 4659 | 22040 | cₛcₛuₛaₛgₛuₛcₛaₛcₛgₛgₛcₛuₛaₛgₛcₛuₛgₛuₛgₛaₛaₛa | 9461 | Anti- |
| | | | | | | | sense |
| 277 | S27 | CUAGUCACGGCUAGCUGUGAAAG | 4660 | 22041 | cₛuₛaₛgₛuₛcₛaₛcₛgₛgₛcₛuₛaₛgₛcₛuₛgₛuₛgₛaₛaₛaₛg | 9462 | Anti- |
| | | | | | | | sense |
| 278 | S27 | UAGUCACGGCUAGCUGUGAAAGG | 4661 | 22042 | uₛaₛgₛuₛcₛaₛcₛgₛgₛcₛuₛaₛgₛcₛuₛgₛuₛgₛaₛaₛaₛgₛg | 9463 | Anti- |
| | | | | | | | sense |
| 279 | S27 | AGUCACGGCUAGCUGUGAAAGGU | 4662 | 22043 | aₛgₛuₛcₛaₛcₛgₛgₛcₛuₛaₛgₛcₛuₛgₛuₛgₛaₛaₛaₛgₛgₛu | 9464 | Anti- |
| | | | | | | | sense |
| 280 | S27 | GUCACGGCUAGCUGUGAAAGGUC | 4663 | 22044 | gₛuₛcₛaₛcₛgₛgₛcₛuₛaₛgₛcₛuₛgₛuₛgₛaₛaₛaₛgₛgₛuₛc | 9465 | Anti- |
| | | | | | | | sense |
| 281 | S27 | UCACGGCUAGCUGUGAAAGGUCC | 4664 | 22045 | uₛcₛaₛcₛgₛgₛcₛuₛaₛgₛcₛuₛgₛuₛgₛaₛaₛaₛgₛgₛuₛcₛc | 9466 | Anti- |
| | | | | | | | sense |
| 282 | S27 | CACGGCUAGCUGUGAAAGGUCCG | 4665 | 22046 | cₛaₛcₛgₛgₛcₛuₛaₛgₛcₛuₛgₛuₛgₛaₛaₛaₛgₛgₛuₛcₛcₛg | 9467 | Anti- |
| | | | | | | | sense |
| 283 | S27 | ACGGCUAGCUGUGAAAGGUCCGU | 4666 | 22047 | aₛcₛgₛgₛcₛuₛaₛgₛcₛuₛgₛuₛgₛaₛaₛaₛgₛgₛuₛcₛcₛgₛu | 9468 | Anti- |
| | | | | | | | sense |
| 284 | S27 | CGGCUAGCUGUGAAAGGUCCGUG | 4667 | 22048 | cₛgₛgₛcₛuₛaₛgₛcₛuₛgₛuₛgₛaₛaₛaₛgₛgₛuₛcₛcₛgₛuₛg | 9469 | Anti- |
| | | | | | | | sense |
| 285 | S27 | GGCUAGCUGUGAAAGGUCCGUGA | 4668 | 22049 | gₛgₛcₛuₛaₛgₛcₛuₛgₛuₛgₛaₛaₛaₛgₛgₛuₛcₛcₛgₛuₛgₛa | 9470 | Anti- |
| | | | | | | | sense |
| 286 | S27 | GCUAGCUGUGAAAGGUCCGUGAG | 4669 | 22050 | gₛcₛuₛaₛgₛcₛuₛgₛuₛgₛaₛaₛaₛgₛgₛuₛcₛcₛgₛuₛgₛaₛg | 9471 | Anti- |
| | | | | | | | sense |
| 287 | S27 | CUAGCUGUGAAAGGUCCGUGAGC | 4670 | 22051 | cₛuₛaₛgₛcₛuₛgₛuₛgₛaₛaₛaₛgₛgₛuₛcₛcₛgₛuₛgₛaₛgₛc | 9472 | Anti- |
| | | | | | | | sense |
| 311 | S27 | GCAUGACUGCAGAGAGUGCUGAU | 4671 | 22052 | gₛcₛaₛuₛgₛaₛcₛuₛgₛcₛaₛgₛaₛgₛaₛgₛuₛgₛcₛuₛgₛaₛu | 9473 | Anti- |
| | | | | | | | sense |
| 312 | S27 | CAUGACUGCAGAGAGUGCUGAUA | 4672 | 22053 | cₛaₛuₛhₛaₛcₛuₛgₛcₛaₛgₛaₛgₛaₛgₛuₛgₛcₛuₛgₛaₛuₛa | 9474 | Anti- |
| | | | | | | | sense |
| 313 | S27 | AUGACUGCAGAGAGUGCUGAUAC | 4673 | 22054 | aₛuₛgₛaₛcₛuₛgₛcₛaₛgₛaₛgₛaₛgₛuₛgₛcₛuₛgₛaₛuₛaₛc | 9475 | Anti- |
| | | | | | | | sense |
| 314 | S27 | UGACUGCAGAGAGUGCUGAUACU | 4674 | 22055 | uₛgₛaₛcₛuₛgₛcₛaₛgₛaₛgₛaₛgₛuₛgₛcₛuₛgₛaₛuₛaₛcₛu | 9476 | Anti- |
| | | | | | | | sense |
| 315 | S27 | GACUGCAGAGAGUGCUGAUACUG | 4675 | 22056 | gₛaₛcₛuₛgₛcₛaₛgₛaₛgₛaₛgₛuₛgₛcₛuₛgₛaₛuₛaₛcₛuₛg | 9477 | Anti- |
| | | | | | | | sense |
| 316 | S27 | ACUGCAGAGAGUGCUGAUACUGG | 4676 | 22057 | aₛcₛuₛgₛcₛaₛgₛaₛgₛaₛgₛuₛgₛcₛuₛgₛaₛuₛaₛcₛuₛgₛg | 9478 | Anti- |
| | | | | | | | sense |
| 317 | S27 | CUGCAGAGAGUGCUGAUACUGGC | 4677 | 22058 | cₛuₛgₛcₛaₛgₛaₛgₛaₛgₛuₛgₛcₛuₛgₛaₛuₛaₛcₛuₛgₛgₛc | 9479 | Anti- |
| | | | | | | | sense |
| 318 | S27 | UGCAGAGAGUGCUGAUACUGGCC | 4678 | 22059 | uₛgₛcₛaₛgₛaₛgₛaₛgₛuₛgₛcₛuₛgₛaₛuₛaₛcₛuₛgₛgₛcₛc | 9480 | Anti- |
| | | | | | | | sense |
| 319 | S27 | GCAGAGAGUGCUGAUACUGGCCU | 4679 | 22060 | gₛcₛaₛgₛaₛgₛaₛgₛuₛgₛcₛuₛgₛaₛuₛaₛcₛuₛgₛgₛcₛcₛu | 9481 | Anti- |
| | | | | | | | sense |
| 320 | S27 | CAGAGAGUGCUGAUACUGGCCUC | 4680 | 22061 | cₛaₛgₛaₛgₛaₛgₛuₛgₛcₛuₛgₛaₛuₛaₛcₛuₛgₛgₛcₛcₛuₛc | 9482 | Anti- |
| | | | | | | | sense |
| 321 | S27 | AGAGAGUGCUGAUACUGGCCUCU | 4681 | 22062 | aₛgₛaₛgₛaₛgₛuₛgₛcₛuₛgₛaₛuₛaₛcₛuₛgₛgₛcₛcₛuₛcₛu | 9483 | Anti- |
| | | | | | | | sense |
| 322 | S27 | GAGAGUGCUGAUACUGGCCUCUC | 4682 | 22063 | gₛaₛgₛaₛgₛuₛgₛcₛuₛgₛaₛuₛaₛcₛuₛgₛgₛcₛcₛuₛcₛuₛc | 9484 | Anti- |
| | | | | | | | sense |
| 157 | HCV+ | CGGAACCGGUGAG | 4683 | 22524 | cₛuₛcₛaₛcc cUGAuGaggccguuaggccGaa Iuuccg B | 9485 | Ino- |
| | | | | | | | zyme |
| 167 | HCV+ | GAGUACACCGGAA | 4684 | 22525 | uₛuₛcₛcₛgg cUCAuGaggccguuaggccGaa Iuacuc B | 9486 | Ino- |
| | | | | | | | zyme |
| 139 | HCV+ | GAGAGCCAUAGUG | 4685 | 22526 | cₛaₛcₛUₛau cUGAuGaggccguuaggccGaa Icucuc B | 9487 | Ino- |
| | | | | | | | zyme |
| 140 | HCV+ | AGAGCCAUAGUGG | 4686 | 22527 | cₛcₛaₛcₛua cUGAuGaggccguuaggccGaa Igcucu B | 9488 | Ino- |
| | | | | | | | zyme |
| 281 | HCV+ | AAGGCCUUGUGGU | 4687 | 22528 | aₛcₛcₛaₛca cUGAuGaggccguuaggccGaa Igccuu B | 9489 | Ino- |
| | | | | | | | zyme |
| 130 | HCV+ | CCCUCCCGGGAGA | 4688 | 22529 | uₛcₛuₛcₛcc cUGAuGaggccguuaggccGaa Igaggg B | 9490 | Ino- |
| | | | | | | | zyme |
| 280 | HCV+ | AAAGGCCUUGUGG | 4689 | 22530 | cₛcₛaₛcₛaa cUGAuGaggccguuaggccGaa Iccuuu B | 9491 | Ino- |
| | | | | | | | zyme |
| 149 | HCV+ | GUGGUCUGCGGAA | 4690 | 22531 | uₛuₛcₛcₛgc cUGAuGaggccguuaggccGaa Iaccac B | 9492 | Ino- |
| | | | | | | | zyme |
| 194 | HCV+ | GGGUCCUUUCUUG | 4691 | 22532 | cₛaₛaₛgₛaa cUGAuGaggccguuaggccGaa Igaccc B | 9493 | Ino- |
| | | | | | | | zyme |
| 255 | HCV+ | GCUAGCCGAGUAG | 4692 | 22533 | cₛuₛaₛcₛuc cUGAuGaggccguuaggccGaa Icuagc B | 9494 | Ino- |
| | | | | | | | zyme |
| 294 | HCV+ | ACUGCCUGAUAGG | 4693 | 22534 | cₛcₛUₛaₛUC cUGAuGaggccguuaggccGaa Igcagu B | 9495 | Ino- |
| | | | | | | | zyme |
| 293 | HCV+ | UACUGCCUGAUAG | 4694 | 22535 | cₛuₛaₛuₛca cUGAuGaggccguuaggccGaa Icagua B | 9496 | Ino- |
| | | | | | | | zyme |
| 290 | HCV+ | UGGUACUGCCUGA | 4695 | 22536 | uₛcₛaₛgₛgc cUGAuGaggccguuaggccGaa Iuacca B | 9497 | Ino- |
| | | | | | | | zyme |
| 169 | HCV+ | GUACACCGGAAUU | 4696 | 22537 | aₛaₛuₛuₛcc cUGAuGaggccguuaggccGaa Iuguac B | 9498 | Ino- |
| | | | | | | | zyme |
| 293 | HCV+ | GUACUGCCUGAUAGG | 4697 | 22544 | cₛcₛuₛaₛuca cUGAuGaggccguuaggccGaa Icaguac B | 9499 | Ino- |
| | | | | | | | zyme |
| 294 | HCV+ | UACUGCCUGAUAGGG | 4698 | 22545 | cₛcₛcₛuₛauc cUGAuGaggccguuaggccGaa Igcagua B | 9500 | Ino- |
| | | | | | | | zyme |
| 281 | HCV+ | AAAGGCCUUGUGGUA | 4699 | 22546 | uₛaₛcₛcₛaca cUGAuGaggccguuaggccGaa Igccuuu B | 9501 | Ino- |
| | | | | | | | zyme |
| 166 | HCV+ | UGAGUACACCGGA | 4700 | 22549 | uₛcₛcₛgₛgu cUGAUGaggccguuaggccGaa Uacuca B | 9502 | Amber- |
| | | | | | | | zyme |
| 168 | HCV+ | AGUACACCGGAAU | 4701 | 22550 | aₛuₛuₛcₛcg cUGAUGaggccguuaggccGaa Uguacu B | 9503 | Amber- |
| | | | | | | | zyme |
| 141 | HCV+ | GAGCCAUAGUGGU | 4702 | 22551 | aₛcₛcₛaₛcu cUGAUGaggccguuaggccGaa Uggcuc B | 9504 | Amber- |
| | | | | | | | zyme |
| 156 | HCV+ | GCGGAACCGGUGA | 4703 | 22552 | uₛcₛaₛcₛcg cUGAUGaggccguuaggccGaa Uuccgc B | 9505 | Amber- |
| | | | | | | | zyme |
| 155 | HCV+ | UGCGGAACCGGUG | 4704 | 22553 | cₛaₛcₛcₛgg cUGAUGaggccguuaggccGaa Uccgca B | 9506 | Amber- |
| | | | | | | | zyme |
| 289 | HCV+ | GUGGUACUGCCUG | 4705 | 22554 | cₛaₛgₛgₛca cUGAUGaggccguuaggccGaa Uaccac B | 9507 | Amber- |
| | | | | | | | zyme |
| 297 | HCV+ | GCCUGAUAGGGUG | 4706 | 22555 | cₛaₛcₛcₛcu cUGAUGaggccguuaggccGaa Ucaggc B | 9508 | Amber- |
| | | | | | | | zyme |
| 166 | HCV+ | GUGAGUACACCGGAA | 4707 | 22556 | uₛuₛcₛcₛggu cUGAUGaggccguuaggccGaa Uacucac B | 9509 | Amber- |
| | | | | | | | zyme |
| 141 | HCV+ | AGAGCCAUAGUGGUG | 4708 | 22557 | gₛaₛcₛcₛacu cUGAUGaggccguuaggccGaa Uggcuc B | 9510 | Amber- |
| | | | | | | | zyme |
| 156 | HCV+ | UGCGGAACCGGUGAG | 4709 | 22558 | cₛuₛcₛaₛccg cUGAUGaggccguuaggccGaa Uuccgca B | 9511 | Amber- |
| | | | | | | | zyme |
| 155 | HCV+ | CUGCGGAACCGGUGA | 4710 | 22559 | uₛcₛaₛcₛcgg cUGAUGaggccguuaggccGaa Uccgcag B | 9512 | Amber- |
| | | | | | | | zyme |
| 289 | HCV+ | UGUGGUACUGCCUGA | 4711 | 22560 | uₛcₛaₛgₛgca cUGAUGaggccguuaggccGaa Uaccaca B | 9513 | Amber- |
| | | | | | | | zyme |
| 297 | HCV+ | UGCCUGAUAGGGUGG | 4712 | 22561 | gₛcₛaₛcₛccu cUGAUGaggccguuaggccGaa Ucaggca B | 9514 | Amber- |
| | | | | | | | zyme |
| 168 | HCV− | GAGUACACCGGAAUU | 4713 | 22562 | aₛaₛuₛuₛccg cUGAUGaggccguuaggccGaa Uguacuc B | 9515 | Amber- |
| | | | | | | | zyme |
| 166 | HCV− | UCCGGUGUACUCA | 4714 | 22563 | uₛgₛaₛgₛua gccgaaaggCgagugaGguGCu accgga B | 9506 | Zin- |
| | | | | | | | zyme |
| 168 | HCV− | AUUCCGGUGUACU | 4715 | 22564 | aₛgₛuₛaₛca gccgaaaggCgagugaGguGCu cggaau B | 9517 | Zin- |
| | | | | | | | zyme |
| 138 | HCV− | ACUAUGGCUCUCG | 4716 | 22565 | gₛgₛaₛgₛag gccgaaaggCgagugaGguGCu cauagu B | 9518 | Zin- |
| | | | | | | | zyme |
| 156 | HCV− | UCACCGGUUCCGG | 4717 | 22566 | gₛcₛgₛgₛaa gccgaaaggCgagugaGguGCu cgguga B | 9519 | Zin- |
| | | | | | | | zyme |
| 236 | HCV− | GCGGGGGCACGCG | 4718 | 22567 | gₛgₛcₛgₛug gccgaaaggCgagugaGguGCu ccccgc B | 9520 | Zin- |
| | | | | | | | zyme |
| 279 | HCV− | CACAAGGCCUUUG | 4719 | 22568 | gₛaₛaₛaₛgg gccgaaaggCgagugaGguGCu cuugug B | 9521 | Zin- |
| | | | | | | | zyme |
| 151 | HCV− | GGUUCCGCAGACG | 4720 | 22569 | gₛgₛuₛcₛug gccgaaaggCgagugaGguGCu ggaacc B | 9522 | Zin- |
| | | | | | | | zyme |
| 292 | HCV− | UAUCAGGCAGUAG | 4721 | 22570 | gₛuₛaₛcₛug gccgaaaggCgagugaGguGCu cugaua B | 9523 | Zin- |
| | | | | | | | zyme |
| 289 | HCV− | CAGGCAGUACCAG | 4722 | 22571 | gₛuₛgₛgₛua gccgaaaggCgagugaGguGCu ugccug B | 9524 | Zin- |
| | | | | | | | zyme |
| 166 | HCV− | UUCCGGUGUACUCAG | 4723 | 22572 | gₛuₛgₛaₛgua gccgaaaggCgagugaGguGCu accggaa B | 9525 | Zin- |
| | | | | | | | zyme |
| 279 | HCV− | CCACAAGGCCUUUCG | 4724 | 22573 | cₛgₛaₛaₛagg gccgaaaggCgagugaGguGCu cuugugg B | 9526 | Zin- |
| | | | | | | | zyme |
| 156 | HCV− | CUCACCGGUUCCGGA | 4725 | 22574 | uₛgₛcₛgₛgaa gccgaaaggCgagugaGguGCu cggugag B | 9527 | Zin- |
| | | | | | | | zyme |
| 138 | HCV− | CACUAUGGCUCUCCG | 4726 | 22575 | gₛgₛgₛaₛgag gccgaaaggCgagugaGguGCu cauagug B | 9528 | Zin- |
| | | | | | | | zyme |
| 151 | HCV− | CGGUUCCGCAGACCA | 4727 | 22576 | uₛgₛgₛuₛcug gccgaaaggCgagugaGguGCu ggaaccg B | 9529 | Zin- |
| | | | | | | | zyme |
| 292 | HCV− | CUAUCAGGCAGUACG | 4728 | 22577 | gₛgₛuₛaₛcug gccgaaaggCgagugaGguGCu cugauag B | 9530 | Zin- |
| | | | | | | | zyme |
| 289 | HCV− | UCAGGCAGUACCACA | 4729 | 22578 | uₛgₛuₛgₛgua gccgaaaggCgagugaGguGCu ugccuga B | 9531 | Zin- |
| | | | | | | | zyme |
| 168 | HCV− | AAUUCCGGUGUACUG | 4730 | 22579 | gₛaₛgₛuₛaca gccgaaaggCgagugaGguGCu cggaauu B | 9532 | Zin- |
| | | | | | | | zyme |
| 163 | HCV− | GGUGUACUCACCG | 4731 | 22580 | cₛgₛgₛuₛga cUGAUGaggccguuaggccGaa Uacacc B | 9533 | Amber- |
| | | | | | | | zyme |
| 159 | HCV− | UACUCACCGGUUG | 4732 | 22581 | gₛaₛaₛcₛcg cUGAUGaggccguuaggccGaa Ugagua B | 9534 | Amber- |
| | | | | | | | zyme |
| 140 | HCV− | CCACUAUGGCUCU | 4733 | 22582 | aₛgₛaₛgₛcc cUGAUGaggccguuaggccGaa Uagugg B | 9535 | Amber- |
| | | | | | | | zyme |
| 281 | HCV− | ACCACAAGGCCUU | 4734 | 22583 | aₛaₛgₛgₛcc cUGAUGaggccguuaggccGaa Uguggu B | 9536 | Amber- |
| | | | | | | | zyme |
| 233 | HCV− | GGGGCACGCCCAA | 4735 | 22584 | uₛuₛgₛgₛgc cUGAUGaggccguuaggccGaa Ugcccc B | 9537 | Amber- |
| | | | | | | | zyme |
| 143 | HCV− | AGACCACUAUGGG | 4736 | 22585 | gₛcₛcₛaₛua cUGAUGaggccguuaggccGaa Uggucu B | 9538 | Amber- |
| | | | | | | | zyme |
| 146 | HCV− | CGCAGACCACUAU | 4737 | 22586 | aₛuₛaₛgₛug cUGAUGaggccguuaggccGaa Ucugcg B | 9539 | Amber- |
| | | | | | | | zyme |
| 195 | HCV− | CCAAGAAAGGACG | 4738 | 22587 | gₛgₛuₛcₛcu cUGAUGaggccguuaggccGaa Ucuugg B | 9540 | Amber- |
| | | | | | | | zyme |
| 194 | HCV− | CAAGAAAGGACCG | 4739 | 22588 | gₛgₛgₛuₛcc cUGAUGaggccguuaggccGaa Uucuug B | 9541 | Amber- |
| | | | | | | | zyme |
| 283 | HCV− | GUACCACAAGGCG | 4740 | 22589 | gₛgₛcₛcₛuu cUGAUGaggccguuaggccGaa Ugguac B | 9542 | Amber- |
| | | | | | | | zyme |
| 286 | HCV− | GCAGUACCACAAG | 4741 | 22590 | cₛuₛuₛgₛug cUGAUGaggccguuaggccGaa Uacugc B | 9543 | Amber- |
| | | | | | | | zyme |
| 296 | HCV− | ACCCUAUCAGGCA | 4742 | 22591 | uₛgₛcₛcₛug cUGAUGaggccguuaggccGaa Uagggu B | 9544 | Amber- |
| | | | | | | | zyme |
| 190 | HCV− | AAAGGACCCGGUG | 4743 | 22592 | gₛaₛcₛcₛgg cUGAUGaggccguuaggccGaa Uccuuu B | 9545 | Amber- |
| | | | | | | | zyme |
| 163 | HCV− | CGGUGUACUCACCGG | 4744 | 22593 | cₛcₛgₛgₛuga cUGAUGaggccguuaggccGaa Ucaccg B | 9546 | Amber- |
| | | | | | | | zyme |
| 140 | HCV− | ACCACUAUGGCUCUG | 4745 | 22594 | gₛaₛgₛaₛgcc cUGAUGaggccguuaggCCGaa Uaguggu B | 9547 | Amber- |
| | | | | | | | zyme |
| 159 | HCV− | GUACUCACCGGUUCG | 4746 | 22595 | gₛgₛaₛaₛccg cUGAUGaggccguuaggccGaa Ugaguac B | 9548 | Amber- |
| | | | | | | | zyme |
| 233 | HCV− | GGGGGCACGCCCAAA | 4747 | 22596 | uₛuₛuₛgₛggc cUGAUGaggccguuaggccGaa Ugccccc B | 9549 | Amber- |
| | | | | | | | zyme |
| 143 | HCV− | CAGACCACUAUGGCU | 4748 | 22597 | aₛgₛcₛcₛaua cUGAUGaggccguuaggccGaa Uggucug B | 9550 | Amber- |
| | | | | | | | zyme |
| 146 | HCV− | CCGCAGACCACUAUG | 4749 | 22598 | cₛaₛuₛaₛgug cUGAUGaggccguuaggccGaa Ucugcgg B | 9551 | Amber- |
| | | | | | | | zyme |
| 195 | HCV− | UCCAAGAAAGGACCG | 4750 | 22599 | gₛgₛgₛuₛccu cUGAUGaggccguuaggccGaa Ucuugga B | 9552 | Amber- |
| | | | | | | | zyme |
| 283 | HCV− | AGUACCACAAGGCCU | 4751 | 22600 | aₛgₛgₛcₛcuu cUGAUGaggccguuaggccGaa Ugguacu B | 9553 | Amber- |
| | | | | | | | zyme |
| 281 | HGV− | UACCACAAGGCCUUU | 4752 | 22601 | aₛaₛaₛgₛgcc cUGAUGaggccguuaggccGaa Uguggua B | 9554 | Amber- |
| | | | | | | | zyme |
| 296 | HCV− | CACCCUAUCAGGCAG | 4753 | 22602 | cₛuₛgₛcₛcug cUGAUGaggccguuaggccGaa Uagggug B | 9555 | Amber- |
| | | | | | | | zyme |
| 286 | HCV− | GGCAGUACCACAAGG | 4754 | 22603 | cₛcₛuₛuₛgug cUGAUGaggccguuaggccGaa Uacugcc B | 9556 | Amber- |
| | | | | | | | zyme |
| 7985 | HCV− | UCUCAGU G UCUUCCA | 4765 | 22719 | uggaaga uGAUg gcauGcacuaugc gCg acugaga B | 9557 | G- |
| | | | | | | | cleav- |
| | | | | | | | er |
| 4832 | HCV− | UGUAUAU G CCUCUCC | 4755 | 22720 | ggagagg uGAUg gcauGcacuaugc gCg auauaca B | 9558 | G- |
| | | | | | | | cleav- |
| | | | | | | | er |
| 4153 | HCV− | ACCGUGU G CCUUAGA | 4756 | 22721 | ucuaagg uGAUg gcauGcacuaugc gCg acacggu B | 9559 | G- |
| | | | | | | | cleav- |
| | | | | | | | er |
| 3200 | HCV− | GUGGAGU G AGGUGGU | 4757 | 22722 | accaccu uGAUg gcauGcacuaugc gCg acuccau B | 9560 | G- |
| | | | | | | | cleav- |
| | | | | | | | er |
| 1682 | HCV− | ACGAGUU G AACCUGU | 4758 | 22723 | acagguu uGAUg gcauGcacuaugc gCg aacucgu B | 9561 | G- |
| | | | | | | | cleav- |
| | | | | | | | er |
| 896 | HCV+ | CCUGUCU G ACCAUCG | 4759 | 22724 | ggauggu uGAUg gcauGcacuaugc gCg agacagg B | 9562 | G- |
| | | | | | | | cleav- |
| | | | | | | | er |
| 2504 | HCV+ | UCCUGUU G CUUUUCC | 4762 | 22725 | ggaaaag uGAUg gcauGcacuaugc gCg aacagga B | 9563 | G- |
| | | | | | | | cleav- |
| | | | | | | | er |
| 2651 | HCV− | UCCUCGU G UUCUUCU | 4763 | 22726 | agaagaa uGAUg gcauGcacuaugc gCg acgagga B | 9564 | G- |
| | | | | | | | cleav- |
| | | | | | | | er |
| 4094 | HCV− | ACAAAGU G CUCGUCC | 4760 | 22727 | ggacgag uGAUg gcauGcacuaugc gCg acuuugu B | 9565 | G- |
| | | | | | | | cleav- |
| | | | | | | | er |
| 8970 | HCV+ | GCCACUU G ACCUACC | 4761 | 22728 | gguaggu uGAUg gcauGcacuaugc gCg aaaguggc B | 9566 | G- |
| | | | | | | | cleav- |
| | | | | | | | er |
| 1200 | HCV+ | CUUCCUG G UCUCUCA | 4789 | 22747 | ugagaga gccgaaaggCgagugaGGuCu gaggaag B | 9567 | Zin- |
| | | | | | | | zyme |
| 1211 | HCV− | CUCAGCU G UUCACCU | 4790 | 22748 | aggugaa gccgaaaggCgagugaGGuCu agcugag B | 9568 | Zin- |
| | | | | | | | zyme |
| 2504 | HCV− | UCCUGUU G CUUUUCC | 4762 | 22749 | ggaaaag gccgaaaggCgagugaGGuCu aacagga B | 9569 | Zin- |
| | | | | | | | zyme |
| 2651 | HCV+ | UCCUCGU G UUCUUCU | 4763 | 22750 | agaagaa gccgaaaggCgagugaGGuCu acgagga B | 9570 | Zin- |
| | | | | | | | zyme |
| 8811 | HCV+ | CACUCCA G UCAACUC | 4764 | 22751 | gaguuga gccgaaaggCgagugaGGuCu uggagug B | 9571 | Zin- |
| | | | | | | | zyme |
| 8594 | HCV− | UCGCCGG G UCCUCUU | 4793 | 22752 | aagagga gccgaaaggCgagugaGGuCu gcggcga B | 9572 | Zin- |
| | | | | | | | zyme |
| 7985 | HCV− | UCUCAGU G UCUUCCA | 4765 | 22753 | uggaaga gccgaaaggCgagugaGGuCu acugaga B | 9573 | Zin- |
| | | | | | | | zyme |
| 6611 | HCV− | CCUCCAG G UACUCCU | 4796 | 22754 | aggagua gccgaaaggCgagugaGGuCu guggagg B | 9574 | Zin- |
| | | | | | | | zyme |
| 5633 | HCV− | UCCACAU G UGCUUCG | 4766 | 22755 | cgaagca gccgaaaggCgagugaGGuCu augugga B | 9575 | Zin- |
| | | | | | | | zyme |
| 821 | HCV− | UCACGCG G UCUUCCA | 4767 | 22756 | uggaaga gccgaaaggCgagugaGGuCu ggcguga B | 9576 | Zin- |
| | | | | | | | zyme |
| 870 | HCV+ | CUCUAUG U UCCUCUU | 4768 | 22775 | aagagga CUGAUGAggccguuaggccGAA Iauagag B | 9577 | Ino- |
| | | | | | | | zyme |
| 1210 | HCV+ | UCUCAGG U GUUCACC | 4769 | 22776 | ggugaac CUGAUGAggccguuaggccGAA Icugaga B | 9578 | Ino- |
| | | | | | | | zyme |
| 2642 | HCV+ | UCCUCUC C UUCCUCG | 4770 | 22777 | cgaggaa CUGAUGAggccguuaggccGAA Iagagga B | 9579 | Ino- |
| | | | | | | | zyme |
| 5726 | HCV+ | UCACAGC C UCCAUCA | 4771 | 22778 | ugaugga CUGAUGAggccguuaggccGAA Icuguga B | 9568 | Ino- |
| | | | | | | | zyme |
| 8142 | HCV+ | CUCCACC C UUCCUCA | 4772 | 22779 | ugaggaa CUGAUGAggccguuaggccGAA Iguggag B | 9581 | Ino- |
| | | | | | | | zyme |
| 7990 | HCV− | UGGUGUG U CAGUGUC | 4773 | 22780 | gacacug CUGAUGAggccguuaggccGAA Iacacca B | 9582 | Ino- |
| | | | | | | | zyme |
| 7813 | HCV− | CUUCGCG U UCAUCUC | 4774 | 22781 | gagauga CUGAUGAggccguuaggccGAA Igcgaag B | 9583 | Ino- |
| | | | | | | | zyme |
| 7137 | HCV− | ACCUCUG U CUCAUCC | 4775 | 22782 | ggaugag CUGAUGAggccguuaggccGAA Iagaggu B | 9584 | Ino- |
| | | | | | | | zyme |
| 6084 | HCV− | UUCAUCC A CUGCACA | 4776 | 22783 | ugugcag CUGAUGAggccguuaggccGAA Igaugaa B | 9585 | Ino- |
| | | | | | | | zyme |
| 2554 | HCV− | CAACAGC A UCAUCCA | 4777 | 22784 | uggauga CUGAUGAggccguuaggccGAA Icuguug B | 9586 | Ino- |
| | | | | | | | zyme |
| 1202 | HCV+ | UCCUCGU C UCUCAGC | 4778 | 22943 | gcugaga CUGAUGAggccguuaggccGAA Acgagga B | 9587 | Ham- |
| | | | | | | | mer- |
| | | | | | | | head |
| 1607 | HCV+ | GGCACAU U AACAGGA | 4779 | 22944 | uccuguu CUGAUGAggccguuaggccGAA Augugcc B | 9588 | Ham- |
| | | | | | | | mer- |
| | | | | | | | head |
| 2639 | HCV+ | GCAUCCU C UCCUUCC | 4780 | 22945 | ggaagga CUGAUGAggccguuaggccGAA Aggaugc B | 9589 | Ham- |
| | | | | | | | mer- |
| | | | | | | | head |
| 6610 | HCV+ | GAGGAGU A CGUGGAG | 4781 | 22946 | cuccacg CUGAUGAggccguuaggccGAA Acuccuc B | 9590 | Ham- |
| | | | | | | | mer- |
| | | | | | | | head |
| 9014 | HCV+ | GCGCAUU U UCACUCC | 4782 | 22947 | ggaguga CUGAUGAggccguuaggccGAA Aaugcgc B | 9591 | Ham- |
| | | | | | | | mer- |
| | | | | | | | head |
| 8605 | HCV− | GACUCGU A GGCUCGC | 4783 | 22948 | gcgagcc CUGAUGAggccguuaggccGAA Acgaguc B | 9592 | Ham- |
| | | | | | | | mer- |
| | | | | | | | head |
| 7983 | HCV− | UCAGUGU C UUCCAGC | 4784 | 22949 | gcuggaa CUGAUGAggccguuaggccGAA Acacuga B | 9593 | Ham- |
| | | | | | | | mer- |
| | | | | | | | head |
| 7136 | HCV− | CCUCUCU C UCAUCCU | 4785 | 22950 | aggauga CUGAUGAggccguuaggccGAA Agagagg B | 9594 | Ham- |
| | | | | | | | mer- |
| | | | | | | | head |
| 6609 | HCV− | UCCACGU A CUCCUCA | 4786 | 22951 | ugaggag CUGAUGAggccguuaggccGAA Acgugga B | 9595 | Ham- |
| | | | | | | | mer- |
| | | | | | | | head |
| 6292 | HCV− | CGUGCAU A UCCAGUC | 4787 | 22952 | gacugga CUGAUGAggccguuaggccGAA Augcacg B | 9596 | Ham- |
| | | | | | | | mer- |
| | | | | | | | head |
| 867 | HCV+ | UUUCUCU A UCUUCCU | 4788 | 22971 | aggaaga GGCTAGCTACAACGA agagaaa B | 9597 | DNA- |
| | | | | | | | zyme |
| 1200 | HCV+ | CUUCCUG G UCUCUCA | 4789 | 22972 | ugagaga GGCTAGCTACAACGA gaggaag B | 9598 | DNA- |
| | | | | | | | zyme |
| 1211 | HCV+ | CUCAGCU G UUCACCU | 4790 | 22973 | aggugaa GGCTAGCTACAACGA agcugag B | 9599 | DNA- |
| | | | | | | | zyme |
| 5730 | HCV+ | AGCCUCC A UCACCAG | 4791 | 22974 | cugguga GGCTAGCTACAACGA ggaggcu B | 9600 | DNA- |
| | | | | | | | zyme |
| 6533 | HCV+ | UCAACGC A UACACCA | 4792 | 22975 | uggugua GGCTAGCTACAACGA gcguuga B | 9601 | DNA- |
| | | | | | | | zyme |
| 8594 | HCV− | UCGCCGG G UCCUCUU | 4793 | 22976 | aagagga GGCTAGCTACAACGA gcggcga B | 9602 | DNA- |
| | | | | | | | zyme |
| 7810 | HCV− | CGCCUUC A UCUCCUU | 4794 | 22977 | aaggaga GGCTAGCTACAACGA gaaggcg B | 9603 | DNA- |
| | | | | | | | zyme |
| 7133 | HCV− | CUCUCUC A UCCUCCU | 4795 | 22978 | aggagga GGCTAGCTACAACGA gagagag B | 9604 | DNA- |
| | | | | | | | zyme |
| 6611 | HCV− | CCUCCAG G UACUCCU | 4796 | 22979 | aggagua GGCTAGCTACAACGA guggagg B | 9605 | DNA- |
| | | | | | | | zyme |
| 2300 | HCV− | CCUCCAA A UCACAAC | 4797 | 22980 | guuguga GGCTAGCTACAACGA uuggagg B | 9606 | DNA- |
| | | | | | | | zyme |
| 195 | HCV+ | GGGUCCU U UCUUGGA | 4556 | 23072 | cₛcₛaₛaₛga cUGAuGaggcgWWagccGaa Aggacc B | 9607 | Ham- |
| | | | | | | | mer- |
| | | | | | | | head |
| 195 | HCV+ | GGGUCCU U UCUUGGA | 45562 | 23076 | WWWWWcₛcₛaₛaₛga cUGAuGaggcgWWWagccGaa Aggacc B | 9608 | Ham- |
| | | | | | | | mer- |
| | | | | | | | head |
| 195 | HCV+ | GGGUCCU U UCUUGGA | 45562 | 23077 | WWWcₛcₛaₛaₛga cUGAuGaggcgWWWagccGaa Aggacc B | 9609 | Ham- |
| | | | | | | | mer- |
| | | | | | | | head |
| 195 | HCV+ | GGGUCCU U UCUUGGA | 45562 | 23086 | cₛcₛaₛaₛga cUGAuGaggcgWWWagccGa Aggacc B | 9610 | Ham- |
| | | | | | | | mer- |
| | | | | | | | head |

lower case = 2′-O-methyl
UPPER CASE = RIBO
B = inverted deoxy abasic
U = 2′-deoxy-2′-amino Uridine
C = 2′-deoxy-2′-amino Cytidine
U = 2′-deoxy-2′-amino Uridine
Z = BRdU (5-bromo-2′-deoxy Uridine)
W = acyclic galactoe-amine linker
UNDERLINE = deoxy nuclectide

TABLE VI
Anti HCV amino containing hammerhead ribozyme and control sequence

| | | HCV 5′UTR | | | Rz Seq |
|---|---|---|---|---|---|
| pos | RPI# | Site | | Core | ID |
| | | | Ribozyme Sequences (5′-3′) | | |
| 621 | 2257 | HCV-62 | gₛcₛgₛugaa cUGAUGaggccguuaggccGaa AcaguagB | Active | 9611 |
| 791 | 2258 | HCV-79 | aₛuₛgₛgcua cUGAUGaggccguuaggccGaa AcgcuuuB | Active | 9612 |
| 811 | 2249 | HCV-81 | cₛcₛaₛuggc cUGAUGaggccguuaggccGaa AgacgcuB | Active | 9613 |
| 1041 | 2259 | HCV-104 | gₛcₛuₛgcac cUGAUGaggccguuaggccGaa AcacucaB | Active | 9614 |
| 1421 | 2250 | HCV-142 | aₛgₛaₛccac cUGAUGaggccguuaggCCGaa AuggcucB | Active | 9615 |
| 1481 | 2251 | HCV-148 | uₛuₛcₛcgca cUGAUGaggccguuaggccGaa AccacuaB | Active | 9616 |
| 1651 | 2260 | HCV-165 | uₛcₛcₛggug cUGAUGaggccguuaggccGaa AcucaccB | Active | 9617 |
| 1921 | 2261 | HCV-192 | aₛaₛgₛaaa9 cUGAUGaggccguuaggccGaa AcccgguB | Active | 9618 |
| 1951 | 2252 | HCV-195 | uₛcₛcₛaaga cUGAUGaggccguuaggccGaa AggacccB | Active | 9619 |
| 1961 | 2262 | HCV-196 | aₛuₛcₛcaag cUGAUGaggccguuaggccGaa AaggaccB | Active | 9620 |
| 2701 | 2263 | HCV-270 | cₛuₛuₛucgc cUGAUGaggccguuaggccGaa AcccaacB | Active | 9621 |
| 2821 | 2264 | HCV-282 | Bₛuₛaₛccac cUGAUGaggccguuaggccGaa AggccuuB | Active | 9622 |
| 3061 | 2265 | HCV-306 | cₛaₛcₛucgc cUGAUGaggccguuaggccGaa AgcacccB | Active | 9623 |
| 3251 | 2253 | HCV-325 | uₛcₛuₛacga cUGAUGaggccguuaggccGaa AccucccB | Active | 9624 |
| 3301 | 2254 | HCV-330 | cₛaₛcₛgguc cUGAUGaggccguuaggccGaa AcgagacB | Active | 9625 |
| | | | Control Sequence | | |
| 79 | 13274 | HCV-79 AC2 | cₛuₛuₛaggu cUAGUGaggccguuaggccGau AguucucB | Attenuated | 9626 |
| 81 | 13271 | HCV-81 AC | uₛcₛuₛgccg cUAGUGaggccguuaggccGau AgugaccB | Attenuated | 9627 |
| 142 | 13270 | HCV-142 AC | aₛaₛcₛccu9 cUAGUGaygcc9uuaggccGau AgcucguB | Attenuated | 9628 |
| 192 | 13272 | HCV-192 AC | aₛgₛuₛagaa cUAGUGaggccguuaggccGaU AgcugccB | Attenuated | 9629 |
| 195 | 13269 | HCV195 AC | gₛaₛuₛucca cUAGUGaggccguuaggccGaU AcgcgacB | Attenuated | 9630 |
| 282 | 13273 | HCV-282 AC | gₛcₛcₛauuc cUAGUGaggccguuaggccGaU AucuggcB | Attenuated | 9631 |
| 330 | 13268 | HCV-330 AC | cₛcₛaₛggcu cUAGUGaggccguuaggccGau AaugcgcB | Attenuated | 9632 |
| 195 | 15291 | HCV-195 BAC3 | uₛcₛcₛaaga cUAGUGacgccguuaggcgGaa AggacccB | Attenuated | 9633 |
| 195 | 15292 | HCV-195 SAC3 | aₛgₛaₛcuac cUAGUGacgccguuaggcgGaa AcccgagB | Attenuated | 9634 |
| 330 | 15294 | HCV-330 BAC | cₛaₛcₛgguc cUAGUGacgccguuaggcgGaa AcgagacB | Attenuated | 9635 |
| 330 | 15295 | HCV-330 SAC | gₛcₛuₛccga cUAGUGacgccguuaggcgGaa AgacacgB | Attenuated | 9636 |

UPPER CASE = RIBO; lower case = 2′-O-methyl; B = inverted deoxyabasic; s = phosphorothioate linkage U = 2′-deoxy-2′-amino uridine

TABLE VII
Anti HCV site 330 antisense nucleic acid and scrambled control sequence

| pos | RPI# | Alias | | Seq ID # |
|---|---|---|---|---|
| | | | Antisense Nucleic Acid | |
| 330 | 17501 | HCV.5-330 | GₛTₛGₛ CₛTₛCₛ AₛTₛGₛ AₛTₛGₛ CₛAₛCₛ GₛGₛTₛ CₛT | 9353 |
| | | antisense | | |
| 330 | 17499 | HCV.5-330 | GₛTₛGₛ CₛTₛcₛ AₛTₛGₛ GₛTₛGₛ cₛAₛcₛ GₛGₛTₛ CₛT | 9637 |
| | | antisense | | |
| | | | Control Sequence | |
| 330 | 17499 | HCV.5-330 scrambled | TₛGₛAₛ TₛcₛAₛ GₛGₛTₛ CₛTₛGₛ CₛTₛGₛ cₛGₛTₛ GₛC | 9638 |
| 330 | 17502 | HCV.5-330 scrambled | TₛGₛAₛTₛcₛAₛGₛGₛTₛcₛTₛGₛcₛTₛGₛcₛAₛTₛGₛG | 9639 |

UPPER CASE = Deoxy Nuclectide
s = phosphorothioate

TABLE VIII
In Vitro Cleavage Data, anti-HCV Enzymatic Nucleic Acid

| | | | | | % Sub- | | | |
|---|---|---|---|---|---|---|---|---|
| | | | | | strate | | | |
| | | | | | Cleav- | | | |
| Seq | | | Site | | ed in | | Seq | Sub- |
| ID | | | (+/ | | 3 | | ID | strate |
| # | RPI# | Motif | −) | Enzymatic Nucleic Acid Sequence | hours | Substrate Sequence | # | RPI# |
| 9587 | 22943 | Hammer- | 1190 | gcugaga CUGAUGAggccguuaggccGAA Acgagga B | 89.67 | UCCUCGU C UCUCAGC B | 9640 | 22897 |
| | head | (+) | | | | | | |
| 9588 | 22944 | Hammer- | 1595 | uccuguu CUGAUGAggccguuaggccGAA Augugga B | 90.33 | GGCACAU U AACAGGA B | 9641 | 22898 |
| | head | (+) | | | | | | |
| 9589 | 22945 | Hammer- | 2627 | ggaagga CUGAUGAggccguuaggccGAA Aggaugc B | 82.54 | GCAUCCU C UCCUUCC B | 9642 | 22899 |
| | head | (+) | | | | | | |
| 9590 | 22946 | Hammer- | 6598 | cuccacg CUGAUGAggccguuaggccGAA Acuccuc B | 78.06 | GAGGAGU A CGUGGAG B | 9643 | 22900 |
| | head | (+) | | | | | | |
| 9591 | 22947 | Hammer- | 9002 | ggaguga CUGAUGAggccguuaggccGAA Aaugcgc B | 81.88 | GCGCAUU U UCACUCC B | 9644 | 22901 |
| | head | (+) | | | | | | |
| 9592 | 22948 | Hammer- | 818 | gcgagcc CUGAUGAggccguuaggccGAA Acgaguc B | 88.34 | GACUCGU A GGCUCGC B | 9645 | 22902 |
| | head | (−) | | | | | | |
| 9593 | 22949 | Hammer- | 1440 | gcuggaa CUGAUGAggccguuaggccGAA Acacuga B | 89.16 | UCAGUGU C UUCCAGC B | 9646 | 22903 |
| | head | (−) | | | | | | |
| 9594 | 22950 | Hammer- | 2287 | aggauga CUGAUGAggccguuaggccGAA Agagagg B | 83.43 | CCUCUCU C UCAUCCU B | 9647 | 22904 |
| | head | (−) | | | | | | |
| 9595 | 22951 | Hammer- | 2814 | ugaggag CUGAUGAggccguuaggccGAA Acgugga B | 83.25 | UCCACGU A CUCCUCA B | 9648 | 22905 |
| | head | (−) | | | | | | |
| 9596 | 22952 | Hammer- | 3131 | gacugga CUGAUGAggccguuaggccGAA Augcacg B | 86.96 | CGUGCAU A UCCAGUC B | 9649 | 22906 |
| | head | (−) | | | | | | |
| 9597 | 22971 | DNAzyme | 855 | aggaaga GGCTAGCTACAACGA agagaaa B | 92.11 | UUUCUCU A UCUUCCU B | 22925 | |
| | | | (+) | | | | | |
| 9598 | 22972 | CNAzyme | 1188 | ugagaga GGCTAGCTACAACGA gaggaag B | 86.38 | CUUCCUC G UCUCUCA B | 22926 | |
| | | | (+) | | | | | |
| 9599 | 22973 | DNAzyme | 1199 | aggugaa GGCTAGCTACAACGA agcugag B | 83.15 | CUCAGCU G UUCACCU B | 22927 | |
| | | | (+) | | | | | |
| 9600 | 22974 | DNAzyme | 5718 | cugguga GGCTAGCTACAACGA ggaggcu B | 57.82 | AGCCUCC A UCACCAG B | 22928 | |
| | | | (+) | | | | | |
| 9601 | 22975 | DNAzyme | 6521 | uggugua GGCTAGCTACAACGA gcguuga B | 75.77 | UCAACGC A UACACCA B | 22929 | |
| | | | (+) | | | | | |
| 9602 | 22976 | DNAzyme | 829 aagagga GGCTAGCTACAACGA gcggcga B | 66.06 | UCGCCGC G UCCUCUU B | 22930 | | |
| | | | (−) | | | | | |
| 9603 | 22977 | DNAzyme | 1613 | aaggaga GGCTAGCTACAACGA gaaggcg B | 71.28 | CGCCUUC A UCUCCUU B | 22931 | |
| | | | (−) | | | | | |
| 9604 | 22978 | DNAzyme | 2290 | aggagga GGCTAGCTACAACGA gagagag B | 61.60 | CUCUCUC A UCCUCCU B | 22932 | |
| | | | (−) | | | | | |
| 96G5 | 22979 | DNAzyme | 2812 | aggagua GGCTAGCTACAACGA guggagg B | 85.53 | CCUCCAC G UACUCCU B | 22933 | |
| | | | (−) | | | | | |
| 9606 | 22980 | DNAzyme | 7123 | guuguga GGCTAGCTACAACGA uuggagg B | 34.60 | CCUCCAA A ACACAAC B | 22934 | |
| | | | (−) | | | | | |
| 9557 | 22719 | G- | 1438 | uggaaga uGAUg gcauGcacuaugc gCg acugaga B | 69.88 | UCUCAGU G UCUUCCA B | | |
| | cleaver | (+) | | | | | | |
| 9558 | 22720 | G- | 4591 | ggagagg uGAUg gcauGcacuaugc gCg auauaca B | 77.74 | UGUAUAU G CCUCUCC B | | |
| | cleaver | (+) | | | | | | |
| 9559 | 22721 | G- | 5270 | ucuaagg uGAUg gcauGcacuaugc gCg acacggu B | 47.37 | ACCGUGU G CCUUAGA B | | |
| | cleaver | (+) | | | | | | |
| 9560 | 22722 | G- | 6223 | accaccu uGAUg gcauccacuaugc gCg acuccac B | 75.84 | GUGGAGU G AGGUGGU B | | |
| | cleaver | (+) | | | | | | |
| 9561 | 22723 | G- | 7741 | acagguu uGAUg gcauccacuaugc gCg aacucgu B | 61.58 | ACGAGUU G AACCUGU B | | |
| | cleaver | (+) | | | | | | |
| 9562 | 22724 | G- | 884 | ggauggu uGAUg gcauGcacuaugc gCg agacagg B | 65.16 | CCUGUCU G ACCAUCC B | | |
| | cleaver | (−) | | | | | | |
| 9563 | 22725 | G- | 2492 | ggaaaag uGAUg gcauGcacuaugc gCg aacagga B | 94.66 | UCCUGUU G CUUUUCC B | | |
| | cleaver | (−) | | | | | | |
| 9564 | 22726 | G- | 2639 | agaagaa uGAUg gcauGcacuaugc gCg acgagga B | 82.14 | UCCUGUU G CUUUUCC B | | |
| | cleaver | (−) | | | | | | |
| 9565 | 22727 | G- | 4082 | ggacgag uGAUg gcauccacuaugc gCg acuuugu B | 67.20 | ACAAAGU G CUCGUCC B | | |
| | cleaver | (−) | | | | | | |
| 9566 | 22728 | G- | 8958 | gguaggu uGAUg gcauccacuaugc gCg aaguggc B | 81.06 | GCCACUU G ACCUACC B | | |
| | cleaver | (−) | | | | | | |
| 9567 | 22747 | Zinzyme | 1188 | ugagaga gccgaaaggCgagugaGGuCu gaggaag B | 66.11 | CUUCCUC G UCUCUCA B | | |
| | | | (+) | | | | | |
| 9568 | 22748 | Zinzyme | 1199 | aggugaa gccgaaaggCgagugaGGuCu agcugag B | 80.28 | CUCAGCU G UUCACCU B | | |
| | | | (+) | | | | | |
| 9569 | 22749 | Zinzyme | 2492 | ggaaaag gccgaaaggCgagugaGGuCu aacagga B | 90.80 | UCCUGUU G CUUUUCC B | | |
| | | | (+) | | | | | |
| 9570 | 22750 | Zinzyme | 2639 | agaagaa gccgaaaggCgagugaGGuCu acgagga B | 80.64 | UCCUCGU G UUCUUCU B | | |
| | | | (+) | | | | | |
| 9571 | 22751 | Zinzyme | 8799 | gaguuga gccgaaaggCgagugaGGuCu uggagug B | 14.85 | CACUCCA G UCAACUC B | | |
| | | | (+) | | | | | |
| 9572 | 22752 | Zinzyme | 829 | aagagga gccgaaaggCgagugaGGuCu gcggcga B | 27.83 | UCGCCGC G UCCUCUU B | | |
| | | | (−) | | | | | |
| 9573 | 22753 | Zinzyme | 1438 | uggaaga gccgaaaggCgagugaGGuCu acugaga B | 89.39 | UCUCAGU G UCUUCCA B | | |
| | | | (−) | | | | | |
| 9574 | 22754 | Zinzyme | 2812 | aggagua gccgaaaggCgagugaGGuCu guggagg B | 50.40 | CCUCCAC G UACUCCU B | | |
| | | | (−) | | | | | |
| 9575 | 22755 | Zinzyme | 3790 | cgaagca gccgaaaggCgagugaGGuCu augugga B | 81.10 | UCCACAU G UGCUUCG B | | |
| | | | (−) | | | | | |
| 9576 | 22756 | Zinzyme | 8602 | uggaaga gccgaaaggCgagugaGGuCu ggcguga B | 73.47 | UCACGCC G UCUUCCA B | | |
| | | | (−) | | | | | |
| 9577 | 22775 | Inozyme | 858 | aagagga CUGAUGAggccguuaggccGAA Iauagag B | 87.74 | CUCUAUC U UCCUCUU B | | |
| | | | (+) | | | | | |
| 9578 | 22776 | Inozyme | 1198 | ggugaac CUGAUGAggccguuaggccGAA Icugaga B | 84.55 | UCUCAGC U UCCUCUU B | | |
| | | | (+) | | | | | |
| 9579 | 22777 | Inozyme | 2630 | cgaggaa CUGAUGAggccguuaggccGAA Iagagga B | 90.12 | UCCUCUC C UUCCUCG B | | |
| | | | (+) | | | | | |
| 9580 | 22778 | Inozyme | 5714 | ugaugga CUGAUGAggccguuaggccGAA Icuguga B | 83.77 | UCACAGC C UCCAUCA B | | |
| | | | (+) | | | | | |
| 9581 | 22779 | Inozyme | 8130 | ugaggaa CUGAUGAggccguuaggccGAA Iguggag B | 82.22 | CUCCACC C UUCCUCA B | | |
| | | | (+) | | | | | |
| 9582 | 22780 | Inozyme | 1433 | gacacug CUGAUGAggccguuaggccGAA Iacacca B | 87.33 | UGGUGUC U CAGUGUC B | | |
| | | | (−) | | | | | |
| 9583 | 22781 | Inozyme | 1610 | gagauga CUGAUGAggccguuaggccGAA Igcgaag B | 70.67 | CUUCGCC U UCAUCUC B | | |
| | | | (−) | | | | | |
| 9584 | 22782 | Inozyme | 2286 | ggaugag CUGAUGAggccguuaggccGAA Iagaggu B | 78.83 | ACCUCUC U CUCAUCC B | | |
| | | | (−) | | | | | |
| 9585 | 22783 | Inozyme | 3339 | ugugcag GUGAUGAggccguuaggccGAA Igaugaa B | 86.93 | UUCAUCC A CUGCACA B | | |
| | | | (−) | | | | | |
| 9586 | 22784 | Inozyme | 6869 | uggauga CUGAUGAggccguuaggccGAA Icuguug B | 90.41 | CAACAGC A UCAUCCA B | | |
| | | | (−) | | | | | |

In vitro cleavage in 50 mM Tris-Cl, pH 8.0, 40 mM Mg²⁺ at 37°, using trace substrate, and enzymatic nucleic acid concentration of 500 nM or greater.
UPPER CASE = RIBO
UNDERLINED = DEOXY
lower case = 2′-O-methyl
B = inverted deoxyabasic
C = 2′-amino C
(+/−) = plus strand/minus strand of HCV genome

All patents and publications mentioned in the specification are indicative of the levels of skill of those skilled in the art to which the invention pertains. All references cited in this disclosure are incorporated by reference to the same extent as if each reference had been incorporated by reference in its entirety individually.

One skilled in the art would readily appreciate that the present invention is well adapted to carry out the objects and obtain the ends and advantages mentioned, as well as those inherent therein. The methods and compositions described herein as presently representative of preferred embodiments are exemplary and are not intended as limitations on the scope of the invention. Changes therein and other uses will occur to those skilled in the art, which are encompassed within the spirit of the invention, are defined by the scope of the claims.

It will be readily apparent to one skilled in the art that varying substitutions and modifications can be made to the invention disclosed herein without departing from the scope and spirit of the invention. Thus, such additional embodiments are within the scope of the present invention and the following claims.

The invention illustratively described herein suitably can be practiced in the absence of any element or elements, limitation or limitations which is not specifically disclosed herein. Thus, for example, in each instance herein any of the terms “comprising”, “consisting essentially of” and “consisting of” can be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments, optional features, modification and variation of the concepts herein disclosed can be resorted to by those skilled in the art, and that such modifications and variations are considered to be within the scope of this invention as defined by the description and the appended claims.

In addition, where features or aspects of the invention are described in terms of Markush groups or other grouping of alternatives, those skilled in the art will recognize that the invention is also thereby described in terms of any individual member or subgroup of members of the Markush group or other group.

Other embodiments are within the following claims.

What we claim is:

1. A compound having Formula I: 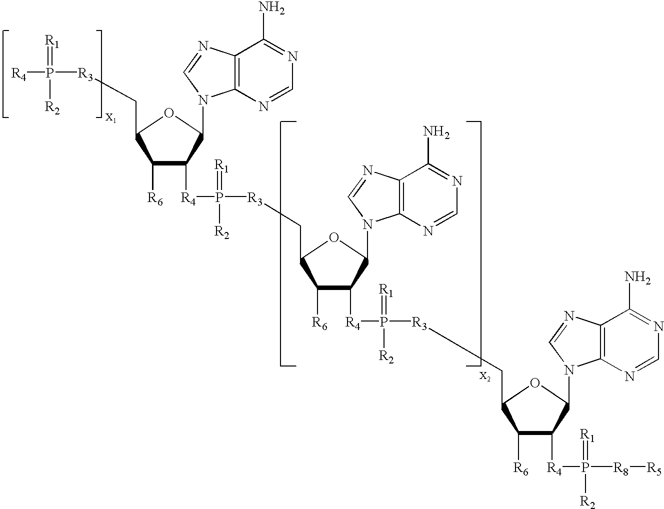
wherein X₁ is an integer of 1, 2, or 3; X₂ is an integer greater than or equal to 1; R₆ independently represents a 3′-ribofuranose sugar moiety; each R₁ and R₂ independently represent non-bridging phosphate moieties; each R₃, R₄ and R₈ independently represent a bridging phosphate moiety; and R₅ represents an alkyl or alkylamine group, or an oligonucleotide comprising any of SEQ ID NOs. 4798-9637, an oligonucleotide having a sequence complementary to a sequence comprising SEQ ID NOs. 1-4556, or abasic moiety.

2. The compound of claim 1, wherein R₆ independently represents H, OH, NH₂, O NH₂, alkyl, S-alkyl, O-alkyl, O-alkyl-S-alkyl, O-alkoxyalkyl, allyl, O-allyl, or fluoro.

3. The compound of claim 1, wherein each R₁ and R₂ independently represent O, alkyl, O-alkyl, or S.

4. The compound of claim 1, wherein R₃, R₄ and R₈ independantly represent O, N, alkyl, fluoroalkyl, or S.

5. The compound of claim 1, wherein said oligonucleotide comprising a sequence complementary to any of SEQ ID NOS. 1-4556 is an enzymatic nucleic acid molecule.

6. The compound of claim 1, wherein said oligonucleotide comprising a sequence complementary to any of SEQ ID NOS. 1-4556 is an antisense nucleic acid molecule.

7. The compound of claim 2, wherein said enzymatic nucleic acid molecule is selected from the group consisting of Hammerhead, Inozyme, G-cleaver, DNAzyme, Amberzyme, and Zinzyme motifs.

8. The compound of claim 4, wherein said Inozyme enzymatic nucleic acid molecule comprises a stem II region of length greater than or equal to 2 base pairs.

9. The compound of claim 5, wherein said enzymatic nucleic acid comprises between 12 and 100 bases complementary to an RNA derived from HCV.

10. The compound of claim 5, wherein said enzymatic nucleic acid comprises between 14 and 24 bases complementary to an RNA derived from HCV.

11. The compound of claim 6, wherein said antisense nucleic acid comprises between 12 and 100 bases complementary to an RNA derived from HCV.

12. The compound of claim 6, wherein said antisense nucleic acid comprises between 14 and 24 bases complementary to an RNA derived from HCV.

13. A pharmaceutical composition comprising the compound of claim 1, in a pharmaceutically acceptable carrier.

14. A mammalian cell comprising a compound of claim 1.

15. The mammalian cell of claim 14, wherein said mammalian cell is a human cell.

16. A method for treatment of cirrhosis, liver failure or hepatocellular carcinoma comprising the step of administering to a patient a compound of claim 1, under conditions suitable for said treatment.

17. A method of treatment of a patient having a condition associated with HCV infection comprising contacting cells of said patient with a compound of claim 1, and further comprising contacting said cells with one or more other therapeutic compounds under conditions suitable for said treatment.

18. A method for inhibiting HCV replication in a mammalian cell comprising the step of administering to said cell the compound of claim 1 under conditions suitable for said inhibition.

19. A method of cleaving a separate RNA molecule comprising contacting the compound of claim 1 with said separate RNA molecule under conditions suitable for the cleavage of said separate RNA molecule.

20. The method of claim 19, wherein said cleavage is carried out in the presence of a divalent cation.

21. The method of claim 20, wherein said divalent cation is Mg²⁺.

22. The method of claim 19, wherein said cleavage is carried out in the presence of a protein nuclease.

23. The method of claim 22, wherein said protein nuclease is an RNAse L nuclease.

24. The compound of claim 1, wherein said compound is chemically synthesized.

25. The compound of claim 1, wherein said oligonucleotide comprises at least one 2′-sugar modification.

26. The compound of claim 1, wherein said oligonucleotide comprises at least one nucleic acid base modification.

27. The compound of claim 1, wherein said oligonucleotide comprises at least one phosphate modification.

28. The method of claim 17, wherein said therapeutic compound is type I interferon.

29. The method of claim 28, wherein said type I interferon and the compound of claim 1 are administered simultaneously.

30. The method of claim 28, wherein said type I interferon and the compound of claim 1 are administered separately.

31. The method of claim 28, wherein said type I interferon is interferon alpha.

32. The method of claim 28, wherein said type I interferon is interferon beta.

33. The method of claim 28, wherein said type I interferon is consensus interferon.

34. The method of claim 28, wherein said type I interferon is polyethylene glycol interferon.

35. The method of claim 28, wherein said type I interferon is polyethylene glycol interferon alpha 2a.

36. The method of claim 28, wherein said type I interferon is polyethylene glycol interferon alpha 2b.

37. The method of claim 28, wherein said type I interferon is polyethylene glycol consensus interferon.

38. The method of claim 17, wherein R₅ in compound 1 is selected from the group consisting of alkyl, alkylamine and abasic moiety and said other therapeutic compound comprises an enzymatic nucleic acid molecule which is targeted against HCV replication.

39. The method of claim 17, wherein R₅ in compound 1 is selected from the group consisting of alkyl, alkylamine and abasic moiety and said other therapeutic compound comprises an antisense nucleic acid molecule which is targeted against HCV replication.

40. A pharmaceutical composition comprising type I interferon and the compound of claim 1, in a pharmaceutically acceptable carrier.

41. The compound of claim 1, wherein said abasic moiety is selected from the group consisting of: 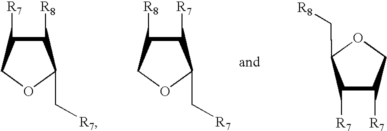
wherein R₈ is R₈ shown in Formula I and R₇ independently represents a ribofuranose sugar moiety.

42. The compound of claim 41, wherein R₇ represents H, OH, NH₂, O—NH₂, alkyl, S-alkyl, O-alkyl, O-alkyl-S-alkyl, O-alkoxyalkyl, allyl, O-allyl, fluoro, oligonucleotide, alkyl, alkylamine or abasic moiety.
